(19) United States
(12) Patent Application Publication
(10) Pub. No.: US 2017/0001990 A1
(43) Pub. Date: Jan. 5, 2017

(54) PIPERIDINE-DIONE DERIVATIVES

(71) Applicant: Genentech, Inc., South San Francisco, CA (US)

(72) Inventors: Jinhua CHEN, Shanghai (CN); Charles Z. DING, Shanghai (CN); Peter DRAGOVICH, South San Francisco, CA (US); Benjamin FAUBER, Austin, TX (US); Zhenting GAO, Shanghai (CN); Sharada S. LABADIE, Sunnyvale, CA (US); Kwong Wah LAI, Shanghai (CN); Hans Edward PURKEY, Burlingame, CA (US); Kirk ROBARGE, San Francisco, CA (US); Binqing WEI, Belmont, CA (US); Aihe ZHOU, San Jose, CA (US)

(73) Assignee: Genentech, Inc., South San Francisco, CA (US)

(21) Appl. No.: 15/266,222

(22) Filed: Sep. 15, 2016

Related U.S. Application Data
(63) Continuation of application No. PCT/EP2015/055495, filed on Mar. 17, 2015.

(30) Foreign Application Priority Data
Mar. 17, 2014 (CN) PCT/CN2014/073509
Aug. 4, 2014 (CN) PCT/CN2014/083613

(51) Int. Cl. C07D 409/14; C07D 401/04; C07D 417/04; C07D 491/107; C07D 211/86; C07D 417/14; C07D 491/048; C07D 405/14; C07D 491/08; C07D 498/08; C07D 409/04; C07D 413/14

(52) U.S. Cl. CPC C07D 409/14; C07D 409/04; C07D 401/04; C07D 417/04; C07D 491/107; C07D 413/14; C07D 417/14; C07D 491/048; C07D 405/14; C07D 491/08; C07D 498/08; C07D 211/86

(57) ABSTRACT

The invention provides novel compounds having the general formula:

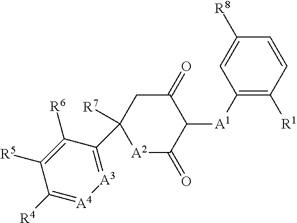

and tautomers and pharmaceutically acceptable salts thereof, wherein A¹, A², A³, A⁴, R¹, R⁴, R⁵, R⁶, R⁷ and R⁸ are as defined herein, compositions including the compounds and methods of using the compounds.

### CROSS REFERENCE TO RELATED APPLICATIONS

This application is a continuation of International Application No. PCT/EP2015/055495, filed on Mar. 17, 2015, which claims priority to Chinese Patent Application No. PCT/CN2014/073509, filed on Mar. 17, 2014 and Chinese Patent Application No. PCT/CN2014/083613, filed Aug. 4, 2014, the contents of which are incorporated herein by reference in their entireties.

### FIELD OF THE INVENTION

The present invention relates to organic compounds useful for therapy and/or prophylaxis in a mammal, and in particular to the inhibition of useful for treating cancer.

Many tumors exhibit altered metabolic characteristics relative to normal, non-transformed tissues (Ward, P. S. et al. Cancer Cell 2012, 21, 297. Vander Heiden, M. G. Nature Rev. Drug Discov. 2011, 10, 671. Zhao, Y. et al. Frontiers in Bioscience 2011, 16, 1844. Kaelin, W. G., Jr. et al. Nature 2010, 465, 562. Tennant, D. A. et al. Nature Rev. Cancer 2010, 10, 267). One example of such altered metabolism is related to the utilization of glucose. Many tumors increase the rate of glucose uptake relative to normal cells and metabolize this nutrient primarily via glycolysis as opposed to the more energy-efficient but oxygen-dependent mitochondrial oxidative phosphorylation process (Vander Heiden, M. G. et al. Science 2009, 324, 1029. Hsu, P. P. et al. Cell 2008, 134, 703). In contrast to normal tissues which typically employ glycolysis only when oxygen supplies limit oxidative phosphorylation (e.g., strenuously working muscle), such glycolytic glucose consumption occurs in cancer cells even in the presence of abundant oxygen levels (Vander Heiden, M. G. et al. Science 2009, 324, 1029. Hsu, P. P. et al. Cell 2008, 134, 703). Originally described by Warburg, (Warburg, O. Science 1956, 123, 309. Bensinger, S. J. et al. Semin. Cell Dev. Biol. 2012, doi: 10.1016/j.semcdb.2012.02.003. Koppenol, W. H. et al. C. V. Nature Rev. Cancer 2011, 11, 325) this “aerobic glycolysis” phenotype is currently viewed as an attractive differentiator between tumors and healthy tissues that can potentially be exploited for the development of new anti-cancer agents (Hamanaka, R. B. et al. J. Exp. Med. 2012, 209, 211. Jones, N. P. et al. Drug Discov. Today 2011, 17, 232. Pelicano, H. et al. Oncogene, 2006, 25, 4633).

Lactate dehydrogenase A (LDHA; also known as LDH-M and LDH-5) is a homotetrameric enzyme which catalyzes the cytosolic conversion of pyruvate to lactate in the final step of glycolysis (Granchi, C. et al. Curr. Med. Chem. 2010, 17, 672. Salaway, J. G. Metabolism at a Glance, 3^{rd} Ed.; Blackwell Publishing: Malden, 2004, pp 10-25. LDHA and B are each homotetramers comprised of M and H subunits, respectively. LDH heterotetramers containing both M and H subunits are also known). This process involves a stereospecific hydride transfer from the reduced form of the associated nicotinamide adenine dinucleotide co-factor (NADH) to the pyruvate ketone moiety. An alternate lactate dehydrogenase isoform (LDHB; also known as LDH-H and LDH-1) can also effect this transformation although it preferentially catalyzes the reverse reaction in which lactate is converted to pyruvate. LDHA is a HIF1α and Myc target gene induced by hypoxia or mutations in VHL, FH, SDH, or the RAS/PI3K/ATK signaling pathways, and elevated LDHA levels are prevalent and associated with poor survival in many cancer indications (Kolev, Y. et al. Ann. Surg. Oncol. 2008, 15, 2336. Koukourakis, M. I. et al. J. Clin. Oncol. 2006, 24, 4301. Koukourakis, M. I. et al. Br. J. Cancer 2003, 89, 877). These observations suggest that LDHA may be an important contributor to the metabolic alterations required for the growth and proliferation of certain tumors. Indeed, shRNA-mediated LDHA knock-down in glycolytic cancer cell lines results in significant inhibition of tumor growth (Seth, P. et al. Neoplasia 2011 13, 60. Qing, G. et al. Cancer Res. 2010, 70, 10351. Fantin, V. R. et al. Cancer Cell 2006, 9, 425) Consistent with the function of LDHA in glycolysis, this growth reduction is more pronounced under hypoxic conditions where cells rely primarily on glycolytic energy production for survival. Similarly, an LDHA inhibitor (FX-11, Le, A et al. Natl. Acad. Sci. 2010, 107, 2037) exhibited in vivo activity against glycolytically dependent tumor xenograft models, although specific inhibition of the LDHA enzyme by this compound was not confirmed in recent experiments by others (Ward, R. A. et al. J. Med. Chem. 2012, 55, 3285). Importantly, humans who lack LDHA through hereditary deficiency display mild phenotypes suggesting that inhibition of the enzyme will not lead to significant intolerable side-effects. 12 Collectively, these data implicate LDHA as an attractive target for the development of new anti-cancer agents for use against hypoxic and/or highly glycolytic tumors.

LDHA inhibitors have been reported in the literature (Le, A. et al. Proc. Natl. Acad. Sci. 2010, 107, 2037. Ward, R. A. et al. J. Med. Chem. 2012, 55, 3285. Granchi, C. et al. J. Med. Chem. 2011, 54, 1599). Some of these molecules were recently described to exhibit ambiguous and/or weak LDHA associations suggesting that the enzyme's biochemical activity may be susceptible to non-specific inhibition effects.

### SUMMARY OF THE INVENTION

In one aspect the invention relates to compounds of Formula (I):

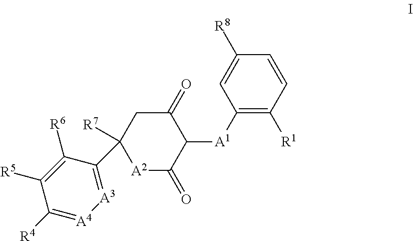

and stereoisomers, geometric isomers, tautomers, and pharmaceutically acceptable salts thereof,

wherein A¹, A², A³, A⁴, R¹, R⁴, R⁵, R⁶, R⁷ and R⁸ are as defined herein. Compounds of Formula (I) can be useful as LDHA inhibitors.

In one aspect the invention relates to tautomers of compounds of Formula (I), such as:

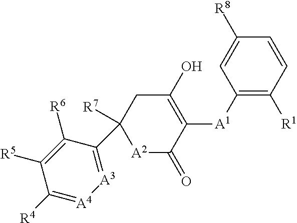

wherein A¹, A², A³, A⁴, R¹, R⁴, R⁵, R⁶, R⁷ and R⁸ are as defined herein. Compounds of Formula (I) can be useful as LDHA inhibitors.

Another aspect of the invention provides a pharmaceutical composition comprising a Formula (I) compound and a pharmaceutically acceptable carrier, glidant, diluent, or excipient.

Another aspect of the invention provides the use of a Formula (I) compound in the manufacture of a medicament for treating cancer.

The invention also relates to methods of using the Formula (I) compounds for in vitro, in situ, and in vivo diagnosis or treatment of mammalian cells, organisms, or associated pathological conditions, such as cancer.

The invention also relates to the use of compounds of Formula (I) and compounds described herein according to the invention in the inhibition of LDHA for the treatment of cancer.

Another aspect of the invention provides a method of treating a disease or disorder which method comprises administering a Formula (I) compound to a patient with cancer.

The methods of treating cancer include where the cancer is breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, pancreatic, myeloid disorders, lymphoma, hairy cells, buccal cavity, naso-pharyngeal, pharynx, lip, tongue, mouth, small intestine, colon-rectum, large intestine, rectum, brain and central nervous system, Hodgkin's, leukemia, bronchus, thyroid, liver and intrahepatic bile duct, hepatocellular, gastric, glioma/glioblastoma, endometrial, melanoma, kidney and renal pelvis, urinary bladder, uterine corpus, uterine cervix, multiple myeloma, acute myelogenous leukemia, chronic lymphoid leukemia, chronic myelogenous leukemia, lymphocytic leukemia, myeloid leukemia, oral cavity and pharynx, non-Hodgkin lymphoma, melanoma, or villous colon adenoma.

Another aspect of the invention provides a kit for treating a condition modulated by the inhibition of, comprising a first pharmaceutical composition comprising a Formula (I) compound; and instructions for use.

Other aspects of the invention include: (i) method for preventing or treating conditions, disorders or diseases mediated by the activation of the LDHA enzyme, in a subject in need of such treatment, which method comprises administering to said subject an effective amount of a compound of Formula (I) or a pharmaceutically acceptable salt thereof, in free form or in a pharmaceutically acceptable salt form as a pharmaceutical, in any of the methods as indicated herein; (ii) a compound of the Formula (I) in free form or in pharmaceutically acceptable salt form for use as a pharmaceutical in any of the methods described herein, in particular for the use in one or more LDHA mediated diseases; (iii) the use of a compound of Formula (I) in free form or in pharmaceutically acceptable salt form in any of the methods as indicated herein, in particular for the treatment of one or more LDHA mediated diseases; (iv) the use of a compound of Formula (I) in free form or in pharmaceutically acceptable salt form in any of the methods as indicated herein, in particular for the manufacture of a medicament for the treatment of one or more LDHA mediated diseases.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

The term “alkyl” as used herein refers to a saturated linear or branched-chain monovalent hydrocarbon radical of one to twelve carbon atoms (C₁-C₁₂), wherein the alkyl radical may be optionally substituted independently with one or more substituent(s) described below. In another embodiment, an alkyl radical is one to eight carbon atoms (C₁-C₈), or one to six carbon atoms (C₁-C₆). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and R² groups as exemplified therein.

The term “C₁-C₁₂-alkoxy” means a C₁-C₁₂-alkyl group, wherein alkyl is as defined herein, that is linked to the rest of a molecule or to another group through an oxygen atom. Illustrative, non limiting examples of alkoxy include methoxy, ethoxy, n-propoxy, isopropoxy and the different butoxy isomers and R¹ groups as exemplified therein.

The expression “(C₁-C₁₂-alkylenyl)ₙ-C₁-C₁₂-alkoxy” means either a (C₁-C₁₂-alkylenyl)-C₁-C₁₂-alkoxy or a C₁-C₁₂-alkoxy group, wherein alkylenyl and alkoxy are as defined herein.

The term “alkylene” or “alkylenyl” as used herein refers to a saturated linear or branched-chain divalent hydrocarbon radical of one to twelve carbon atoms (C₁-C₁₂), wherein the alkylene radical may be optionally substituted independently with one or more substituent(s) described below. In another embodiment, an alkylene radical is one to eight carbon atoms (C₁-C₈), or one to six carbon atoms (C₁-C₆). Examples of alkylene groups include, but are not limited to, methylene (—CH₂—), ethylene (—CH₂CH₂—), propylene (—CH₂CH₂CH₂—), and R¹ groups as exemplified therein.

“Aryl” means a monovalent aromatic hydrocarbon radical of 6-20 carbon atoms (C₆-C₂₀) or C₆-C₂₀-aryl, derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic ring. Typical aryl groups include, but are not limited to, radicals derived from benzene (phenyl), substituted benzenes, naphthalene, anthracene, biphenyl, indenyl, indanyl, 1,2-dihydronaphthalene, 1,2,3,4-tetrahydronaphthyl, and the like. Aryl groups are optionally substituted independently with one or more substituent(s) described herein. Further non limiting examples of aryl groups can be found in the definition of R¹ herein.

“aryloxy” as used herein denotes an —O-aryl group, wherein aryl is as defined herein. Non-limiting examples of —O-aryl groups are —O-phenyl and —O-naphthyl groups.

The term “cyanoalkyl” as used herein refers to an alky group as defined herein that is substituted by one or more cyano group, for example one cyano group. In certain embodiments “cyanoalkyl” are C₁-C₁₂-cyanoalkyl groups. In other embodiments “cyanoalkyl” are C₁-C₆-cyanoalkyl groups, for example cyanomethyl and cyanoethyl.

The terms “carbocycle”, “carbocyclyl”, “carbocyclic ring” and “cycloalkyl” refer to a monovalent non-aromatic, saturated or partially unsaturated ring having 3 to 12 carbon atoms (C₃-C₁₂) as a monocyclic ring or 7 to 12 carbon atoms as a bicyclic ring. Partially unsaturated rings can also be designated as cycloalkenyl rings. Bicyclic carbocycles having 7 to 12 atoms can be arranged, for example, as a bicyclo[4,5], [5,5], [5,6] or [6,6] system, and bicyclic carbocycles having 9 or 10 ring atoms can be arranged as a bicyclo[5,6] or [6,6] system, or as bridged systems such as bicyclo[2.2.1]heptane, bicyclo[2.2.2]octane and bicyclo[3.2.2]nonane. Examples of monocyclic carbocycles or cycloalkyls include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, adamantanyl, and R² groups as exemplified therein.

The term “halo” denotes chloro, iodo, fluoro and bromo, In an embodiment halo are fluoro, chloro and bromo, and yet in another embodiment fluoro and chloro.

The term “haloalkyl” denotes an alkyl group as defined above wherein at least one of the hydrogen atoms of the alkyl group is replaced by a halogen atom, preferably fluoro or chloro, most preferably fluoro. Examples of haloalkyl include C₁-C₁₂-haloalkyl groups, but are not limited to, methyl, ethyl, propyl, isopropyl, isobutyl, sec-butyl, tert-butyl, pentyl or n-hexyl wherein one or more hydrogen atoms are replaced by Cl, F, Br or I atom(s), as well as those haloalkyl groups specifically illustrated by the examples herein below. Among the preferred haloalkyl groups are monofluoro-, difluoro- or trifluoro-methyl, -ethyl or -propyl, for example 3,3,3-trifluoropropyl, 2-fluoroethyl, 2,2,2-trifluoroethyl, fluoromethyl, trifluoromethyl. The term “C₁-C₁₂-haloalkyl” means a haloalkyl group having 1 to 12 carbon atoms, wherein the haloalkyl is as defined herein.

The term “haloalkoxy” denotes an alkoxy group as defined herein wherein at least one of the hydrogen atoms of the alkoxy group is replaced by a halogen atom, preferably fluoro or chloro, most preferably fluoro. Examples of haloalkoxy include C₁-C₁₂-haloalkoxy groups, but are not limited to, methoxy, ethoxy, propyloxy, isopropyloxy, isobutyloxy, sec-butyloxy, tert-butyloxy, pentyloxy or n-hexyloxy wherein one or more hydrogen atoms are replaced by Cl, F, Br or I atom(s), as well as those haloalkoxy groups specifically illustrated by the examples herein below. Among the preferred haloalkoxy groups are monofluoro-, difluoro- or trifluoro-methoxy, -ethoxy or -propyloxy, for example 3,3,3-trifluoropropyloxy, 2-fluoroethoxy, 2,2,2-trifluoroethoxy, fluoromethoxy, trifluoromethoxy. In a certain embodiment C₁-C₁₂-haloalkoxy groups are C₁-C₆-haloalkoxy groups.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituent(s) described below. Examples of heterocyclyl groups are 4 to 10 membered heterocyclyl, i.e. heterocyclyl groups comprising 2 to 9 carbon atoms and 1, 2, 3 or 4 heteroatoms selected from N, O, P, and S. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, piperidonyl, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydroisoquinolinyl, tetrahydroisoquinolinyl, pyrazolidinylimidazolinyl, imidazolidinyl, 2-oxa-5-azabicyclo[2.2.2]octane, 3-oxa-8-azabicyclo[3.2.1]octane, 8-oxa-3-azabicyclo[3.2.1]octane, 6-oxa-3-azabicyclo[3.1.1]heptane, 2-oxa-5-azabicyclo[2.2.1]heptane, 3-azabicyco[3.1.0]hexanyl, 3-azabicyclo[4.1.0]heptanyl, azabicyclo[2.2.2]hexanyl, 3H-indolyl quinolizinyl and N-pyridyl ureas.

Spiro moieties are also included within the scope of this definition. Examples of a heterocyclic group wherein 2 ring carbon atoms are substituted with oxo (═O) moieties are pyrimidinonyl and 1,1-dioxo-thiomorpholinyl. The heterocycle groups herein are optionally substituted independently with one or more substituent(s) described herein.

The term “heteroaryl” refers to a monovalent aromatic radical of 5-, 6-, or 7-membered rings, and includes fused ring systems (at least one of which is aromatic) of 5-20 atoms containing one or more heteroatoms independently selected from nitrogen, oxygen, and sulfur. Examples of heteroaryl groups include 5 to 10 membered heteroaryls which denotes monocyclic of bicyclic heteroaryl having 2 to 9 carbon atoms and one or more heteroatoms independently selected from nitrogen, oxygen, and sulfur, for example, 1, 2, 3 or 4 heteroatoms independently selected from nitrogen, oxygen, and sulfur. Examples of heteroaryl groups include 5 or 6 membered heteroaryls which denotes monocyclic of bicyclic heteroaryl having 2 to 5 carbon atoms and one or more heteroatoms independently selected from nitrogen, oxygen, and sulfur, for example, 1, 2, 3 or 4 heteroatoms independently selected from nitrogen, oxygen, and sulfur. Non limiting examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituent(s) described herein, for example alkyl, alkoxy, cyano, halo, oxo, NH₂, OH, hydroxyalkyl, amido groups. Further examples of heteroaryl groups and of possible substituents can be found in the definition of R².

The term “heteroaryloxy” as used herein means an —O-heteroaryl, wherein heteroaryl is as defined herein.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Ring nitrogen atoms of the heterocycle or heteroaryl groups may be bonded with oxygen to form N-oxides.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, benzimidazole, position 2 of a isoindole, or isoindoline, position 4 of a morpholine, and position 9 of a carbazole, or β-carboline.

The term “hydroxy” denotes a group of formula —OH.

The term “hydroxyalkyl” denotes an alkyl group as defined above wherein at least one of the hydrogen atoms of the alkyl group is replaced by a hydroxy group. Examples of hydroxyalkyl include, but are not limited to, methyl, ethyl, propyl, isopropyl, isobutyl, sec-butyl, tert-butyl, pentyl or n-hexyl wherein one or more hydrogen atoms are replaced by OH, as well as those hydroxyalkyl groups specifically illustrated by the examples herein below. The term “C₁-C₁₂-hydroxyalkyl” means a hydroxyalkyl group having 1 to 12 carbon atoms, wherein hydroxyalkyl is as defined herein.

Oxo denotes a group of formula ═O.

The expression “one or more substituent” denotes a substitution by 1, 2, 3, 4, 5, 6, 7, 8, 9, 10, 11 or 12 substituent(s) that can be independently selected from the list following this expression. In an embodiment, one or more substituent(s) denotes 1, 2, 3, 4 or 5 substituents. In an embodiment, one or more substituent(s) denotes 1, 2 or 3 substituents.

The terms “treat” and “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” refers to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, multiple myeloma, acute myelogenous leukemia, chronic lymphoid leukemia, chronic myelogenous leukemia, lymphocytic leukemia, myeloid leukemia, oral cavity and pharynx, non-Hodgkin lymphoma, melanoma, and villous colon adenoma.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space. Stereoisomers include enantiomers and diastereomers.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography. Diastereomers include geometric isomers, cis/trans and E/Z isomers, and atropisomers.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons. As stated above, the compounds of Formula (I) also covers tautomers thereof, such as depicted in the following formulae:

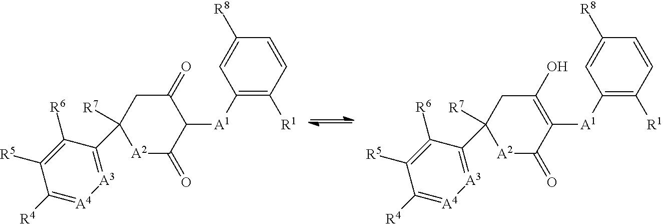

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition must be compatible chemically and/or toxicologically, with the other ingredients comprising a formulation, and/or the mammal being treated therewith.

A “solvate” refers to an association or complex of one or more solvent molecules and a compound of the invention. Examples of solvents that form solvates include, but are not limited to, water, isopropanol, ethanol, methanol, DMSO, ethylacetate, acetic acid, and ethanolamine.

The terms “compound of this invention,” and “compounds of the present invention” and “compounds of Formula (I)” include compounds of Formulas (I), (I-a) and (I-a-I), specific compounds described herein and stereoisomers, tautomers, solvates, metabolites, and pharmaceutically acceptable salts and prodrugs thereof. As stated above, particular tautomers of the compounds of Formula (I) are as depicted below:

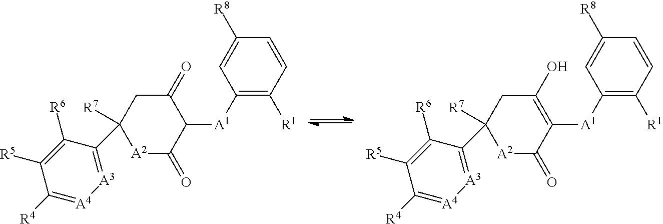

Any formula or structure given herein, including Formula (I) compounds, is also intended to represent hydrates, solvates, and polymorphs of such compounds, and mixtures thereof.

Any formula or structure given herein, including Formula (I) compounds, is also intended to represent isotopically labeled forms of the compounds as well as unlabeled forms. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to 2H (deuterium, D), 3H (tritium), 11C, 13C, 14C, 15N, 18F, 31P, 32P, 35S, 36Cl, and 125I. Various isotopically labeled compounds of the present invention, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the formula (I). The concentration of such a heavier isotope, specifically deuterium, may be defined by an isotopic enrichment factor. In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this invention any atom specifically designated as a deuterium (D) is meant to represent deuterium.

### Inhibitors of LDHA

In one aspect, the invention relates to compounds of Formula (I):

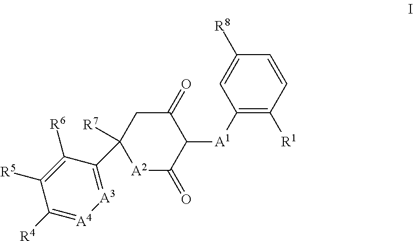

and stereoisomers, tautomers, and pharmaceutically acceptable salts thereof, wherein:

A¹ is O, CH₂, or S;

A² is NH or N—C₁-C₃-alkyl;

A³ is N or CR²;

A⁴ is N or CR³, provided that A³ and A⁴ are not N at the same time;

R¹ is Cl, NO₂, or CN;

R² and R⁶ are independently selected from the group consisting of H, halo, hydroxy, C₁-C₆-hydroxyalkyl, and NH₂;

R³ and R⁵ are independently selected from the group consisting of:
H;
hydroxy;
halo;
—C₁-C₆-alkyl-R^{f};
—C₁-C₆-alkenyl-R^{f};
—C₁-C₆-alkoxy-R^{c};
—NR^{a}R^{b};
—NR^{a}—(C₁-C₆-alkyl)-R^{d};
—NR^{a}—S(O)₂-(4 to 10 membered heterocycloalkyl);
—NR^{a}—(C₃-C₈-cycloalkyl), which cycloalkyl is unsubstituted or substituted by C₁-C₆-alkyl or a C₁-C₃-alkylene bridge;
—NR^{a}-aryl, which aryl is unsubstituted or substituted by one or more substituent(s) selected from the group consisting of:
halo, hydroxy, —NH₂, C₁-C₆-alkyl, C₁-C₆-alkoxy, C₁-C₆-haloalkyl, C₁-C₆-hydroxyalkyl, C₁-C₆-haloalkoxy and C₃-C₈-cycloalkyl;
—NR^{a}-(4 to 10 membered heterocycloalkyl), which heterocycloalkyl is unsubstituted or substituted by one or more substituent(s) selected from the group consisting of: C₁-C₆-alkyl, C₁-C₆-hydroxyalkyl, or —CO-alkyl;
—NR^{a}-(5 or 6 membered heteroaryl), which heteroaryl is unsubstituted or substituted by one or more substituent(s) selected from the group consisting of: halo, —NR^{a}R^{b} and C₁-C₆-alkyl;
—NR^{a}(CO)—C₁-C₆-alkyl;
—NR^{a}(CO)-aryl;
—NR^{a}(CO)-(5 or 6 membered heteroaryl);
—NR^{a}(CO)O—C₁-C₆-alkyl;
—S-(alkyl)ₙ-R^{h};
—S(O)₂-aryl, which aryl is unsubstituted or substituted by one or more halo;
—C(O)—R^{e};
—C(O)NR^{a}—(C₁-C₆-alkyl)ₙ-R^{g};
—C(O)NR^{a}—C₁-C₆-alkoxy;
—O—C₃-C₈-cycloalkyl, which cycloalkyl is unsubstituted or substituted by one or more substituent(s) selected from the group consisting of: halo or hydroxy, C₁-C₆-alkyl, C₁-C₆-alkoxy, C₁-C₆-haloalkoxy, C₁-C₆-alkoxyaryl, C₁-C₆-haloalkyl, C₁-C₆-hydroxyalkyl, NR^{a}R^{b}, aryl, C₁-C₆-akyl-aryl, 5 or 6 membered heteroaryl, and —(C₁-C₆-alkyl)-(C₁-C₆-alkoxy);
—O-aryl, which aryl is unsubstituted or substituted by one or more substituent(s) selected from the group consisting of:
halo, C₁-C₆-alkyl, C₁-C₆-alkoxy, C₁-C₆-alkyl-C₁-C₆-alkoxy, C₁-C₆-haloalkyl, C₁-C₆-haloalkoxy, C₁-C₆-hydroxyalkyl, —S—C₁-C₆-akyl, —C₁-C₆-alkyl-C₃-C₈-cycloalkyl, C₁-C₆-alkoxy-C₃-C₈-cycloalkyl, C₁-C₆-alkyl-(4 to 10 membered heterocycloalkyl), C₁-C₆-alkyl-(5 or 6 membered heterocycloalkyl), or 5 or 6 membered heteroaryl unsubstituted or substituted by one or more substituent(s) selected from the group consisting of: C₁-C₆-alkyl, —(C₁-C₆-alkyl)-(C₁-C₆-alkoxy), C₁-C₆-haloalkoxy and a C₁-C₆-alkylene bridge;
—O-(4 to 10 membered heterocycloalkyl), which heterocycloalkyl is unsubstituted or substituted by one or more substituent(s) selected from the group consisting of:
halo, hydroxy, C₁-C₆-alkyl, C₁-C₆-hydroxyalkyl and —C(O)—C₁-C₆-alkyl;
—O-(5 to 10 membered heteroaryl), which heteroaryl is unsubstituted or substituted by halo, C₁-C₆-alkyl, C₁-C₆-hydroxyalkyl, or —NR^{a}(CO)—C₁-C₆-akyl;
C₃-C₈-cycloalkyl, which cycloalkyl may be fused to a phenyl;
aryl unsubstituted or substituted by one or more substituent(s) selected from the group consisting of:
halo, hydroxy, —C(O)OH, C₁-C₆-hydroxyalkyl, C₁-C₆-alkoxy, —S(O)₂—NH(alkyl) and —S(O)₂—N(alkyl)₂;
4 to 10 membered heterocycloalkyl unsubstituted or substituted by one or more substituent(s) selected from the group consisting of:
halo, C₁-C₆-alkyl, —C(O)—C₃-C₈-cycloalkyl, oxo and 5 or 6 membered heterocycloalkyl;
5 to 10 membered heteroaryl unsubstituted or substituted by one or more substituent(s) selected from the group consisting of:
hydroxy, —NR^{a}R^{b}, C₁-C₆-alkyl, C₁-C₆-hydroxyalkyl, and 4 to 10 membered heterocycloalkyl;

R⁴ is:
cyano,
halo,
hydroxy,
NR^{a}R^{b},
C₁-C₆-alkyl,
C₁-C₆-haloalkyl,
C₁-C₆-hydroxyalkyl,
C₁-C₆-alkoxy unsubstituted or substituted by hydroxy, C₁-C₆-alkoxy or NR^{a}R^{b},
—(C₁-C₆-alkyl)ₙ-(C₃-C₈-cycloalkyl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl, —C(O)—C₁-C₆-alkyl, —C(O)—C₁-C₆-cycloalkyl; —C(O)-(5 or 6 membered heterocycloalkyl);
—(C₁-C₆-alkyl)ₙ-(C₃-C₈-cycloalkenyl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl, —C(O)—C₁-C₆-alkyl, —C(O)—C₁-C₆-cycloalkyl and —C(O)-(5 or 6 membered heterocycloalkyl);
—(C₁-C₆-alkyl)ₙ-(5 or 6 membered heteroaryl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl and —C(O)—C₁-C₆-alkyl, —C(O)—C₁-C₆-cycloalkyl and —C(O)-(5 or 6 membered heterocycloalkyl);
—(C₁-C₆-alkyl)ₙ-(4 to 10 membered heterocycloalkyl) unsubstituted or substituted by one or more substituent(s) selected from the group consisting of: halo, hydroxy, cyano, —NR^{a}R^{b}, C₁-C₆-alkyl, C₁-C₆-alkoxy, C₁-C₆-haloalkyl, C₁-C₆-hydroxyalkyl, —C(O)OH, a C₁-C₄-alkylene bridge, —C(O)—C₁-C₆-alkyl, —C(O)—C₃-C₈-cycloalkyl, —C(O)-aryl, —C(O)(4 to 10 membered heterocycloalkyl) and —C(O)-(5 or 6 membered heterocycloalkyl);

R⁷ is aryl, a 5 or 6 membered heterocycle or 5 or 6 membered heteroaryl which aryl, heterocycle or heteroaryl is unsubstituted or substituted by one or more substituent(s) selected from the group consisting of halo, C₁-C₆-alkyl, C₃-C₈-cycloalkyl, —O-aryl, —S-aryl, —NH-aryl, and —(C₁-C₆-alkyl)ₙ-aryl;

or R⁶ and R⁷ together with the carbon atoms to which they are attached form a 5 membered ring selected from a cycloalkyl or heterocycloalkyl having 5 ring members;

R⁸ is OH, —NR^{a}R^{b}, C₁-C₆-alkoxy or —C(O)O—C₁-C₆-alkyl;

or R² and R³ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;

or R³ and R⁴ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;

or R⁴ and R⁵ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;

or R⁵ and R⁶ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;

R^{a} is H or C₁-C₆-alkyl;

R^{b} is H or C₁-C₆-alkyl;

R^{c} is H, hydroxy, halo, —NR^{a}R^{b}, C₁-C₆-alkoxy, C₁-C₆-alkenyl, 4 to 6 membered heterocycloalkyl unsubstituted or substituted by oxo or C₁-C₆-alkyl, 5 or 6 membered heteroaryl unsubstituted or substituted by C₁-C₆-alkyl, or C₃-C₈-cycloalkyl unsubstituted or substituted by one or more substituent(s) selected from the group consisting of:
halo, C₁-C₆-alkyl or C₁-C₆-hydroxyalkyl, aryl unsubstituted or substituted by halo, 4 to 9 membered heterocycloalkyl unsubstituted or substituted by oxo or C₁-C₆-alkyl, and 5 or 6 membered heteroaryl unsubstituted or substituted by C₁-C₆-alkyl;

R^{d} is H, hydroxy, C₁-C₆-alkyl, C₃-C₈-cycloalkyl or aryl unsubstituted or substituted by one or more substituent(s) selected from the group consisting of halo and —NR^{a}—S(O)₂—N(C₁-C₆-alkyl)₂;

R^{e} is C₁-C₆-alkyl, aryl, C₃-C₈-cycloalkyl, 5 to 9 membered heterocycloalkyl or 5 or 6 membered heteroaryl and wherein said aryl, C₃-C₈-cycloalkyl, 5 to 9 membered heterocycloalkyl or 5 or 6 membered heteroaryl is unsubstituted or substituted by one or more substituent(s) selected from the group consisting of: halo, C₁-C₆-alkoxy, C₁-C₆-alkyl and C₁-C₆-haloalkyl;

R^{f} is H, C₃-C₈-cycloalkyl, 4 to 10 membered heterocycloalkyl, aryl, or 5 or 6 membered heteroaryl, which cycloalkyl, heterocycloalkyl, aryl, or heteroaryl is unsubstituted or substituted by one or more substituent(s) selected from the group consisting of halo, C₁-C₆-haloalkyl, C₁-C₆-alkyl, C₁-C₆-alkoxy and C₁-C₆-hydroxyalkyl;

R^{g} is C₁-C₆-alkoxy, C₃-C₈-cycloalkyl, aryl, 5 or 6 membered heteroaryl, 5 to 9 membered heterocycloalkyl, wherein said aryl, C₃-C₈-cycloalkyl, 5 to 9 membered heterocycloalkyl or 5 or 6 membered heteroaryl is unsubstituted or substituted by one or more substituent(s) selected from the group consisting of halo, C₁-C₆-alkoxy and C₁-C₆-hydroxyalkyl;

R^{h} is aryl, 5 or 6 membered heteroaryl, 4 to 10 membered heterocycloalkyl, C₃-C₈-cycloalkyl, each of which is unsubstituted or substituted by halo;

n is 0 or 1.

In an embodiment, the invention relates to compounds of Formula (I) can be:

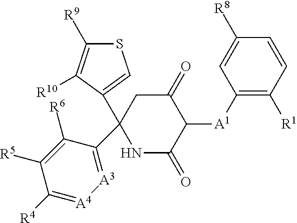

and stereoisomers, geometric isomers, tautomers, and pharmaceutically acceptable salts thereof, wherein A¹, A³, A⁴, R¹, R⁴, R⁵, R⁶, R⁸, R⁹ and R¹⁰ are as described herein.

In an embodiment, the invention relates to compounds of Formula (I) can be:

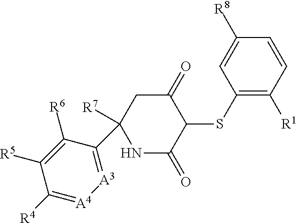

and stereoisomers, geometric isomers, tautomers, and pharmaceutically acceptable salts thereof, wherein A³, A⁴, R¹, R⁴, R⁵, R⁶, R⁷ and R⁸ are as described herein.

In an embodiment, the invention relates to compounds of Formula (I) can be:

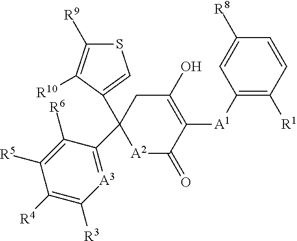

wherein A¹, A², A³, R¹, R³, R⁴, R⁵, R⁶, R⁸, R⁹ and R¹¹ are as described herein.

In an embodiment, the invention relates to compounds of Formula (I) can be:

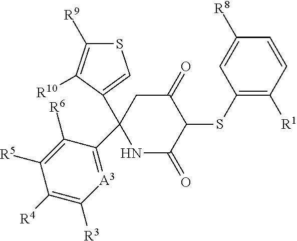

In an embodiment, the invention relates to compounds of Formula (I) can be:

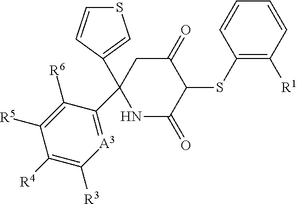

and stereoisomers, geometric isomers, tautomers, and pharmaceutically acceptable salts thereof, wherein A³, R¹, R³, R⁴, R⁵ and R⁶ are as described herein are as described herein.

In an embodiment, the invention relates to compounds of Formula (I) can be:

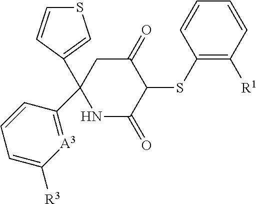

and stereoisomers, geometric isomers, tautomers, and pharmaceutically acceptable salts thereof, wherein A³, R¹ and R³ are as described herein are as described herein.

In an embodiment, the invention relates to compounds of Formula (I) can be:

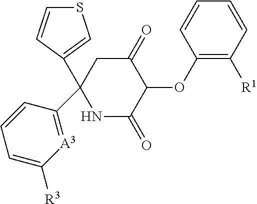

and stereoisomers, geometric isomers, tautomers, and pharmaceutically acceptable salts thereof, wherein A³, R¹ and R³ are as described herein are as described herein.

In an embodiment, the invention relates to compounds of Formula (I) can be:

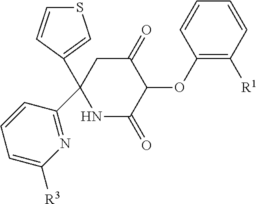

and stereoisomers, geometric isomers, tautomers, and pharmaceutically acceptable salts thereof, wherein R¹ and R³ are as described herein are as described herein.

In an embodiment, the invention relates to compounds of Formula (I) can be:

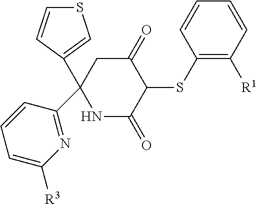

and stereoisomers, geometric isomers, tautomers, and pharmaceutically acceptable salts thereof, wherein R¹ and R³ are as described herein are as described herein.

In an embodiment, the invention relates to compounds of Formula (I) can be:

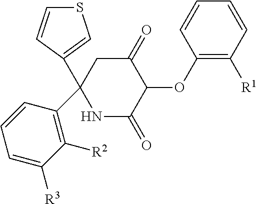

and stereoisomers, geometric isomers, tautomers, and pharmaceutically acceptable salts thereof, wherein R¹, R² and R³ are as described herein are as described herein.

In an embodiment, the invention relates to compounds of Formula (I) can be:

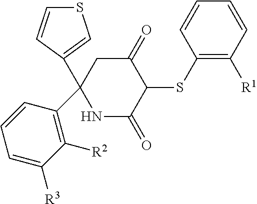

and stereoisomers, geometric isomers, tautomers, and pharmaceutically acceptable salts thereof, wherein R¹, R² and R³ are as described herein are as described herein.

In an embodiment, the compounds of Formula (I) and stereoisomers, geometric isomers, tautomers, and pharmaceutically acceptable salts thereof, wherein:

A is O or S;

A² is NH or N—C₁-C₃-alkyl;

A³ is N or CR²;

R¹ is Cl, NO₂, or CN;

R² and R⁶ are independently selected from the group consisting of H, halo, hydroxy and NH₂;

R³ and R⁵ are independently selected from the group consisting of:
H;
hydroxy;
halo;
—C₁-C₆-alkyl-R^{f}, wherein R^{f} is 4 to 10 membered heterocycloalkyl, aryl, or 5 or 6 membered heteroaryl, which C₃-C₈-cycloalkyl, 5 to 9 membered heterocycloalkyl, aryl, or 5 or 6 membered heteroaryl is unsubstituted or substituted by one or more substituent(s) selected from the group consisting of:
halo, C₁-C₆-alkoxy and C₁-C₆-hydroxyalkyl;
—C₁-C₆-alkoxy-R^{c}, wherein R^{c} is H, hydroxy, halo, —NR^{a}R^{b}, C₁-C₆-alkoxy, C₁-C₆-alkenyl, C₃-C₈-cycloalkyl unsubstituted or substituted by one or more substituent(s) selected from the group consisting of:
halo, C₁-C₆-alkyl or C₁-C₆-hydroxyalkyl, aryl unsubstituted or substituted by halo, 4 to 9 membered heterocycloalkyl unsubstituted or substituted by oxo or C₁-C₆-alkyl, and 5 or 6 membered heteroaryl unsubstituted or substituted by C₁-C₆-alkyl;
—NR^{a}R^{b}, wherein R^{a} and R^{b} are independently selected from H or C₁-C₆-alkyl;
—NR^{a}—(C₁-C₆-alkyl)-R^{d}, wherein R^{a} is H or C₁-C₆-alkyl and R^{d} is H, hydroxy, C₁-C₆-alkyl, C₃-C₈-cycloalkyl or aryl unsubstituted or substituted by one or more substituent(s) selected from the group consisting of:
halo and —NR^{a}—S(O)₂—N(C₁-C₆-alkyl)₂;
—NR^{a}—S(O)₂-(4 to 10 membered heterocycloalkyl), wherein R^{a} is H or C₁-C₆-alkyl;
—NR^{a}—(C₃-C₈-cycloalkyl), wherein R^{a} is H or C₁-C₆-alkyl and which cycloalkyl is unsubstituted;
—NR^{a}-aryl, wherein R^{a} is H or C₁-C₆-alkyl and which aryl is unsubstituted or substituted by one or more substituent(s) selected from the group consisting of:
halo, C₁-C₆-alkoxy, C₁-C₆-haloalkyl, and C₁-C₆-hydroxyalkyl;
—NR^{a}-(4 to 10 membered heterocycloalkyl), wherein R is H or C₁-C₆-alkyl;
—NR^{a}-(5 or 6 membered heteroaryl), wherein R^{a} is H or C₁-C₆-alkyl and which heteroaryl is unsubstituted or substituted by one or more substituent(s) selected from the group consisting of:
halo, —NH₂ or C₁-C₆-alkyl;
—NR^{a}(CO)O—C₁-C₆-alkyl, wherein R^{a} is H or C₁-C₆-alkyl;
—C(O)—R^{e}, wherein R^{e} is aryl and wherein said aryl is substituted by halo, or C₁-C₆-haloalkyl;
—C(O)NR^{a}—(C₁-C₆-alkyl)ₙ-R^{g}, wherein R^{a} is H or C₁-C₆-alkyl and R^{g} is C₁-C₆-alkoxy, C₃-C₈-cycloalkyl;
—O—C₃-C₈-cycloalkyl, which cycloalkyl is unsubstituted or substituted by halo or hydroxy, C₁-C₆-alkyl, C₁-C₆-alkoxy, which alkoxy is unsubstituted or substituted by C₁-C₆-alkoxyaryl, C₁-C₆-haloalkyl;
—O-aryl, which aryl is unsubstituted or substituted by one or more substituent(s) selected from the group consisting of:
halo, C₁-C₆-alkyl, C₁-C₆-alkoxy, C₁-C₆-haloalkyl, C₁-C₆-haloalkoxy, C₁-C₆-hydroxyalkyl, —S—C₁-C₆-akyl, —C₁-C₆-alkyl-C₃-C₈-cycloalkyl, 4 to 10 membered heterocycloalkyl, 5 or 6 membered heteroaryl unsubstituted or substituted by C₁-C₆-alkyl, and C₁-C₆-alkylene bridge;
—O-(4 to 10 membered heterocycloalkyl), which heterocycloalkyl is unsubstituted or substituted by one or more substituent(s) selected from the group consisting of:
hydroxyl, C₁-C₆-hydroxyalkyl, —C(O)—C₁-C₆-alkyl;
—O-(5 to 10 membered heteroaryl), which heteroaryl is unsubstituted or substituted by halo, or —NR^{a}(CO)—C₁-C₆-akyl;
aryl substituted by one or more —S(O)₂—N(alkyl)₂;
4 to 10 membered heterocycloalkyl unsubstituted or substituted by one or more 5 or 6 membered heterocycloalkyl;
5 to 10 membered heteroaryl unsubstituted or substituted by one or more 4 to 10 membered heterocycloalkyl;

R⁴ is:
H,
hydroxy,
C₁-C₆-alkoxy unsubstituted or substituted by hydroxy or C₁-C₆-alkoxy,
—(C₁-C₆-alkyl)ₙ-(C₃-C₈-cycloalkyl),
—(C₁-C₆-alkyl)ₙ-(C₃-C₈-cycloalkenyl),
—(C₁-C₆-alkyl)-(4 to 10 membered heterocycloalkyl) unsubstituted or substituted by one or more substituent(s) selected from the group consisting of:
halo, C₁-C₆-alkyl, or —C(O)—C₁-C₆-alkyl;

R⁷ is 5 or 6 membered heteroaryl which is unsubstituted or substituted by one or more substituent(s) selected from the group consisting of:
halo, alkyl, or —O-aryl, —S-aryl, —NH-aryl, —(C₁-C₆-alkyl)-aryl;

R⁸ is OH, —NH₂, C₁-C₆-alkoxy, —C(O)O—C₁-C₆-alkyl;

or R² and R³ together with the atoms to which they are attached form a naphthyl or 9 or 10 membered heteroaryl, each of which is unsubstituted or substituted by one or more substituent(s) selected from the group consisting of:
halo, hydroxy, —NH₂, —NH(C₁-C₆-alkyl), —N(C₁-C₆-alkyl)₂, C₁-C₆-alkyl, C₁-C₆-alkoxy, and C₁-C₆-haloalkyl;

or R³ and R⁴ together with the atoms to which they are attached form a naphthyl or 9 or 10 membered heteroaryl, each of which is unsubstituted or substituted by one or more substituent(s) selected from the group consisting of:
halo, hydroxy, —NH₂, —NH(C₁-C₆-alkyl), —N(C₁-C₆-alkyl)₂, C₁-C₆-alkyl, C₁-C₆-alkoxy, and C₁-C₆-haloalkyl;

or R⁴ and R⁵ together with the atoms to which they are attached form a naphthyl or 9 or 10 membered heteroaryl, each of which is unsubstituted or substituted by one or more substituent(s) selected from the group consisting of:
halo, hydroxy, —NH₂, —NH(C₁-C₆-alkyl), —N(C₁-C₆-alkyl)₂, C₁-C₆-alkyl, C₁-C₆-alkoxy, C₁-C₆-haloalkyl;

or R⁵ and R⁶ together with the atoms to which they are attached form a naphthyl or 9 or 10 membered heteroaryl, each of which is unsubstituted or substituted by one or more substituent(s) selected from the group consisting of:
halo, hydroxy, —NH₂, —NH(C₁-C₆-alkyl), —N(C₁-C₆-alkyl)₂, C₁-C₆-alkyl, C₁-C₆-alkoxy and C₁-C₆-haloalkyl;

n is 0 or 1.

Unless specifically stated otherwise herein, all of the following embodiments can be combined with one another:

In an embodiment A¹ is O. In an embodiment A¹ is S. In an embodiment A¹ is CH₂.

In an embodiment A² is NH. In an embodiment A² is N—C₁-C₃-alkyl.

In an embodiment A³ is N. In an embodiment A³ is CR².

In an embodiment A⁴ is N. In an embodiment A⁴ is CR³.

In an embodiment, A³ is CR² and A⁴ is CR³. In an embodiment, A³ is NH and A⁴ is CR³. In one embodiment A³ is CR² and A⁴ is NH.

In an embodiment R¹ is Cl. In an embodiment R¹ is NO₂. In an embodiment R¹ is CN.

In an embodiment R² is H. In an embodiment R² is halo. In an embodiment R² is hydroxy. In an embodiment R² is C₁-C₆-hydroxyalkyl. In an embodiment R² is NH₂. In an embodiment R² is halo. In an embodiment R² is hydroxy. In an embodiment R² is C₁-C₆-hydroxyalkyl.

In an embodiment R³ or R⁵ is H. In an embodiment R³ or R⁵ is hydroxy. In an embodiment R³ or R⁵ is halo. In an embodiment R³ or R⁵ is —C₁-C₆-alkyl-R^{f}, wherein R^{f} is as defined herein. In an embodiment R³ or R⁵ is —C₁-C₆-alkenyl-R^{f}, wherein R^{f} is as defined herein. In an embodiment R³ or R⁵ is —C₁-C₆-alkoxy-R^{c}, wherein R^{c} is as defined herein. In an embodiment R³ or R⁵ is —NR^{a}R^{b}, wherein R^{a} and R^{b} are as defined herein. In an embodiment R³ or R⁵ is —NR^{a}—(C₁-C₆-alkyl)-R^{d}, wherein R^{a} and R^{d} are as defined herein. In an embodiment R³ or R⁵ is —NR^{a}—S(O)₂-(4 to 10 membered heterocycloalkyl), wherein R¹ is as defined herein. In an embodiment R³ or R⁵ is —NR^{a}—(C₃-C₈-cycloalkyl), wherein R^{a} is as defined herein and the cycloalkyl is unsubstituted or substituted by C₁-C₆-alkyl. In an embodiment R³ or R⁵ is —NR^{a}-aryl, wherein R^{a} is as defined herein and the aryl is unsubstituted or substituted by one or more substituent(s) selected from the group consisting of:
halo, hydroxy, —NH₂, C₁-C₆-alkyl, C₁-C₆-alkoxy, C₁-C₆-haloalkyl, C₁-C₆-hydroxyalkyl, C₁-C₆-haloalkoxy and C₃-C₈-cycloalkyl.

In an embodiment R³ or R⁵ is —NR^{a}-(4 to 10 membered heterocycloalkyl), wherein R^{a} is as defined herein and the heterocycloalkyl is unsubstituted or substituted by one or more substituent(s) selected from the group consisting of: C₁-C₆-alkyl, C₁-C₆-hydroxyalkyl, or —CO-alkyl.

In an embodiment R³ or R⁵ is —NR^{a}-(5 or 6 membered heteroaryl), wherein R^{a} is as defined herein and the heteroaryl is unsubstituted or substituted by one or more substituent(s) selected from the group consisting of: halo, —NR^{a}R^{b} and C₁-C₆-alkyl.

In an embodiment R³ or R⁵ is —NR^{a}(CO)—C₁-C₆-alkyl wherein R^{a} is as defined herein.

In an embodiment R³ or R⁵ is —NR^{a}(CO)-(aryl).

In an embodiment R³ or R⁵ is —NR^{a}(CO)-(5 or 6 membered heteroaryl).

In an embodiment R³ or R⁵ is —NR^{a}(CO)O—C₁-C₆-alkyl wherein R^{a} is as defined herein.

In an embodiment R³ or R⁵ is —S-(alkyl)ₙ-R^{h} and R^{h} is as defined herein.

In an embodiment R³ or R⁵ is —S(O)₂-aryl, which aryl is unsubstituted or substituted by one or more halo.

In an embodiment R³ or R⁵ is —C(O)—R^{e} and R^{e} is as defined herein.

In an embodiment R³ or R⁵ is —C(O)NR—(C₁-C₆-alkyl)-R^{g}, wherein R^{a} and R^{g} are as defined herein.

In an embodiment R³ or R⁵ is —O—C₃-C₈-cycloalkyl, which cycloalkyl is unsubstituted or substituted by halo or hydroxy, C₁-C₆-alkyl, C₁-C₆-alkoxy, which alkoxy is unsubstituted or substituted by halo, C₁-C₆-alkoxyaryl, C₁-C₆-haloalkyl, aryl, C₁-C₆-akyl-aryl, 5 or 6 membered heteroaryl, C₁-C₆-haloalkoxy, C₁-C₆-hydroxyalkyl, NR^{a}R^{b}, —(C₁-C₆-alkyl)-(C₁-C₆-alkoxy).

In an embodiment R³ or R⁵ is —O-aryl, which aryl is unsubstituted or substituted by one or more substituent(s) selected from the group consisting of halo, C₁-C₆-alkyl, C₁-C₆-alkoxy, C₁-C₆-haloalkyl, C₁-C₆-haloalkoxy, C₁-C₆-hydroxyalkyl, —S—C₁-C₆-akyl, —C₁-C₆-alkyl-C₃-C₈-cycloalkyl, C₁-C₆-alkyl-4 to 10 membered heterocycloalkyl, 5 or 6 membered heteroaryl unsubstituted or substituted by one or more substituent(s) selected from the group consisting of: C₁-C₆-alkyl, —(C₁-C₆-alkyl)-(C₁-C₆-alkoxy), C₁-C₆-haloalkoxy, C₁-C₆-alkylene bridge.

In an embodiment R³ or R⁵ is —O-(4 to 10 membered heterocycloalkyl), which heterocycloalkyl is unsubstituted or substituted by one or more substituent(s) selected from the group consisting of halo, hydroxy, C₁-C₆-hydroxyalkyl and —C(O)—C₁-C₆-alkyl.

In an embodiment R³ or R⁵ is —O-(5 to 10 membered heteroaryl), which heteroaryl is unsubstituted or substituted by halo, or —NR^{a}(CO)—C₁-C₆-akyl and R^{a} is as defined herein.

In an embodiment R³ or R⁵ is C₃-C₈-cycloalkyl, which cycloalkyl may be fused to a phenyl.

In an embodiment R³ or R⁵ is aryl unsubstituted or substituted by one or more substituent(s) selected from the group consisting of halo, hydroxy, —C(O)OH, C₁-C₆-hydroxyalkyl, C₁-C₆-alkoxy, —S(O)₂—NH(alkyl) and —S(O)₂—N(alkyl)₂.

In an embodiment R³ or R⁵ is 4 to 10 membered heterocycloalkyl unsubstituted or substituted by one or more 5 or 6 membered heterocycloalkyl.

In an embodiment R³ or R⁵ is 5 to 10 membered heteroaryl unsubstituted or substituted by one or more substituent(s) selected from the group consisting of hydroxy, —NR^{a}R^{b}, C₁-C₆-alkyl, C₁-C₆-hydroxyalkyl, and 4 to 10 membered heterocycloalkyl.

In an embodiment R³ or R⁵ is —NR^{a}—S(O)₂-(4 to 10 membered heterocycloalkyl), for example:

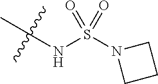 [SMILES: *NS(=O)(=O)N1CCC1]

In an embodiment R³ or R⁵ is —S(O)₂-aryl, which aryl is unsubstituted or substituted by one or more halo, for example:

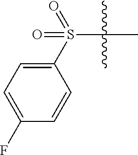 [SMILES: *S(=O)(=O)c1ccc(F)cc1]

In an embodiment R³ or R⁵ is C₃-C₈-cycloalkyl which cycloalkyl may be fused to a phenyl, or which may be partially unsaturated for example:

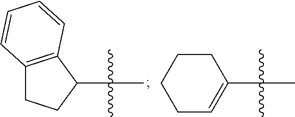 [SMILES: *C1CCc2ccccc21 ; *C1=CCCCC1]

In an embodiment R³ or R⁵ is NR^{a}—(C₁-C₆-alkyl)-R^{d}, wherein R^{d} is C₃-C₈-cycloalkyl, for example:

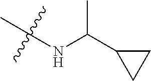 [SMILES: *NC(C)C1CC1]

In an embodiment R³ or R⁵ is C₁-C₆-alkenyl-R^{f}, wherein R^{f} is C₃-C₈-cycloalkyl, for example:

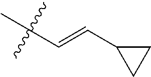 [SMILES: */C=C/C1CC1]

In an embodiment R³ or R⁵ is aryl, for example phenyl unsubstituted or substituted by one or more halo, hydroxy, —C(O)OH, C₁-C₆-hydroxyalkyl, C₁-C₆-alkoxy, —S(O)₂—NH(alkyl) and —S(O)₂—N(alkyl)₂, for example:

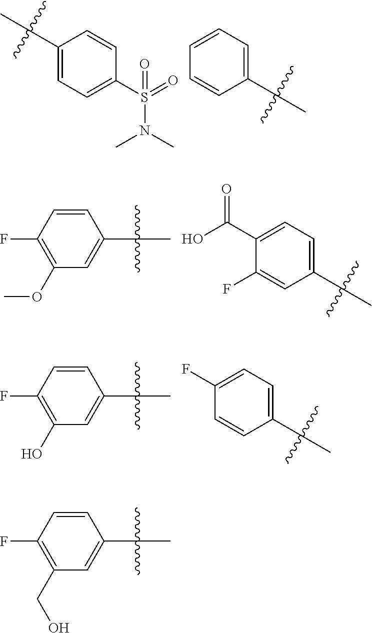

In an embodiment R³ or R⁵ is —NR^{a}-aryl, for example, —NR^{a}-phenyl, which aryl or phenyl is unsubstituted or substituted by one or more halo, C₁-C₆-alkoxy, C₁-C₆-haloalkyl, C₁-C₆-hydroxyalkyl, C₃-C₈-cycloalkyl and R^{a} is H or C₁-C₆-alkyl, for example:

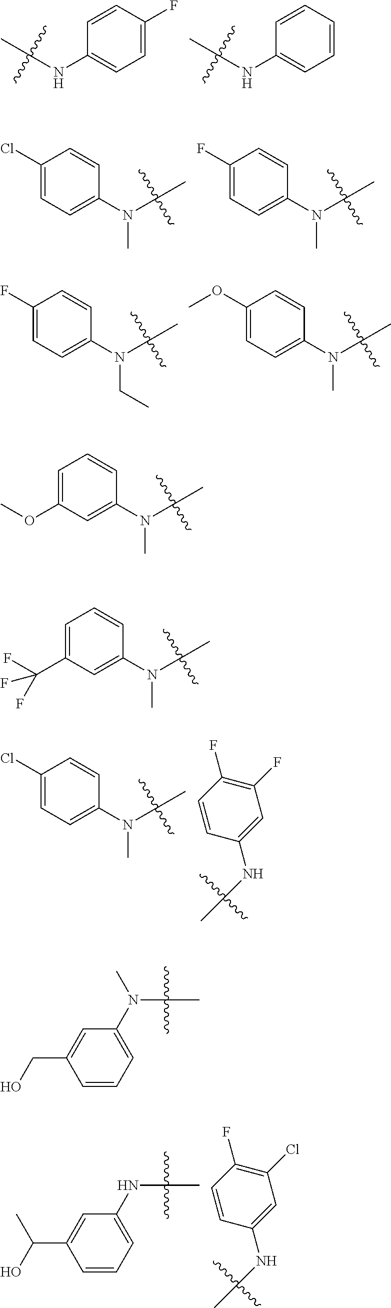

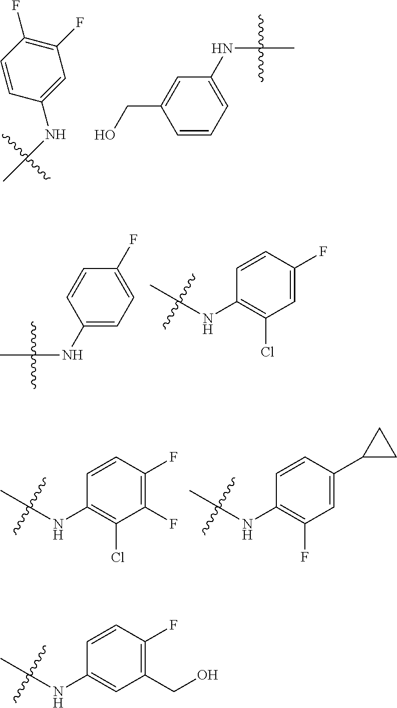

In an embodiment R³ or R⁵ is —O-aryl, for example —O-phenyl, which aryl or phenyl is unsubstituted or substituted by one or more: halo, C₁-C₆-alkyl, —S—C₁-C₆-akyl, C₁-C₆-haloalkyl, C₁-C₆-alkoxy, C₁-C₆-alkoxy-C₃-C₈-cycloalkyl, C₁-C₆-haloalkoxy, C₁-C₆-hydroxyalkyl, C₁-C₆-alkyl-C₁-C₆-alkoxy, C₁-C₆-alkyl-(5 or 6 membered heterocycloalkyl), 5 or 6 membered heterocycloalkyl which 5 or 6 membered heteroaryl is unsubstituted or substituted by C₁-C₆-alkyl, C₁-C₆-haloalkoxy, C₁-C₆-alkylene bridge, naphthalene partially hydrogenated which is unsubstituted or substituted by halo for example:

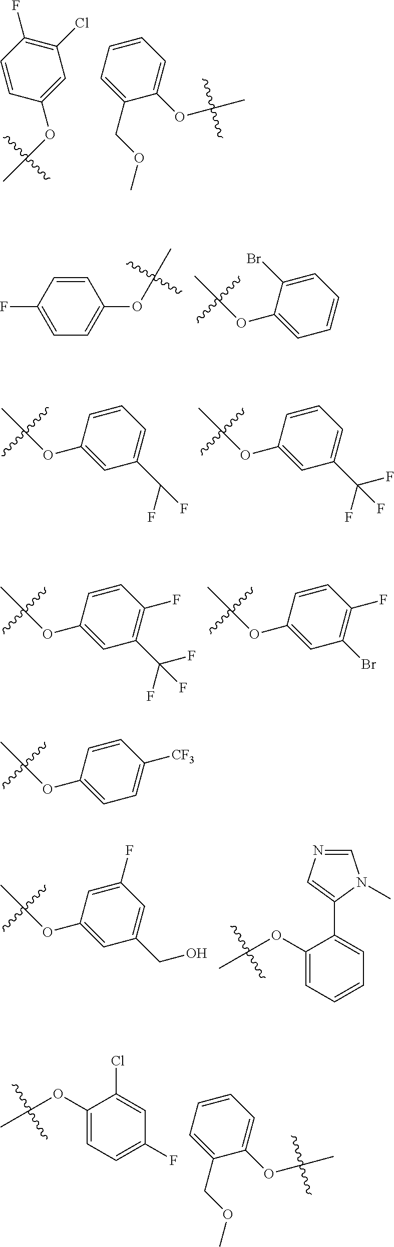

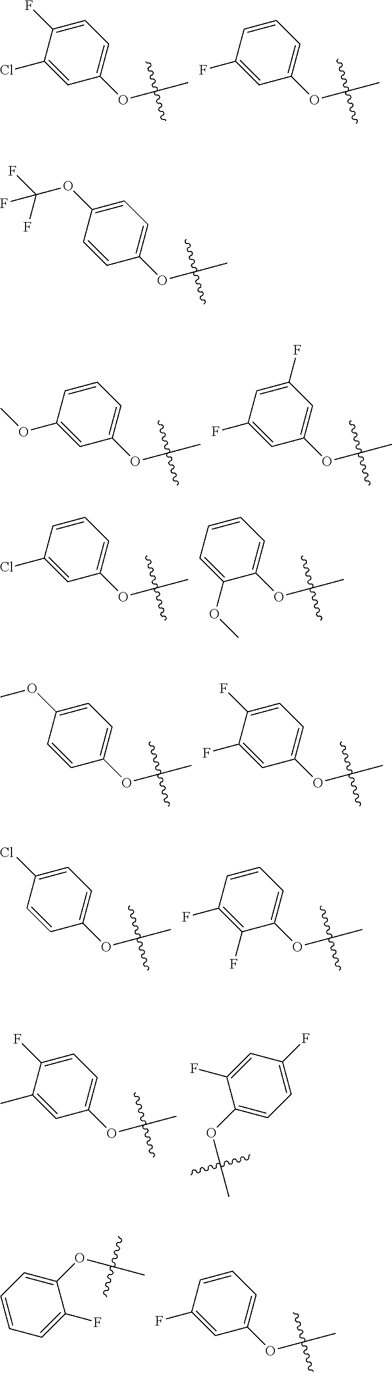

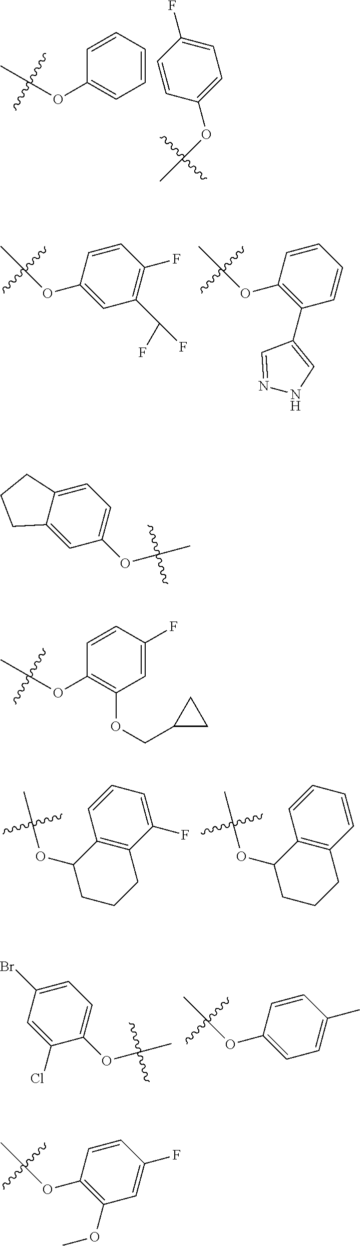

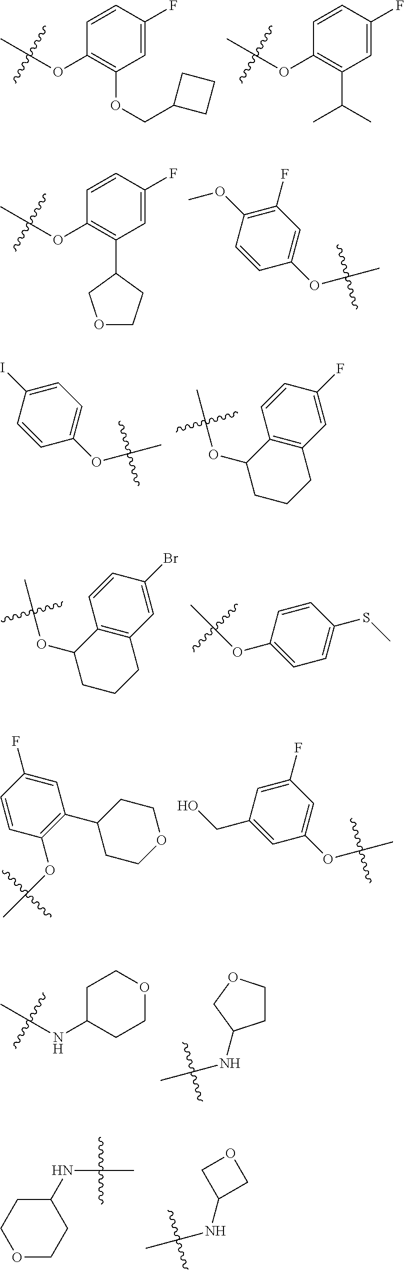

In an embodiment R³ or R⁵ is —NR^{a}-(5 or 6 membered heterocycloalkyl), for example:

In an embodiment R³ or R⁵ is —NR^{a}-(5 or 6 membered heteroaryl), which heteroaryl is unsubstituted or substituted by halo or C₁-C₆-alkyl, for example:

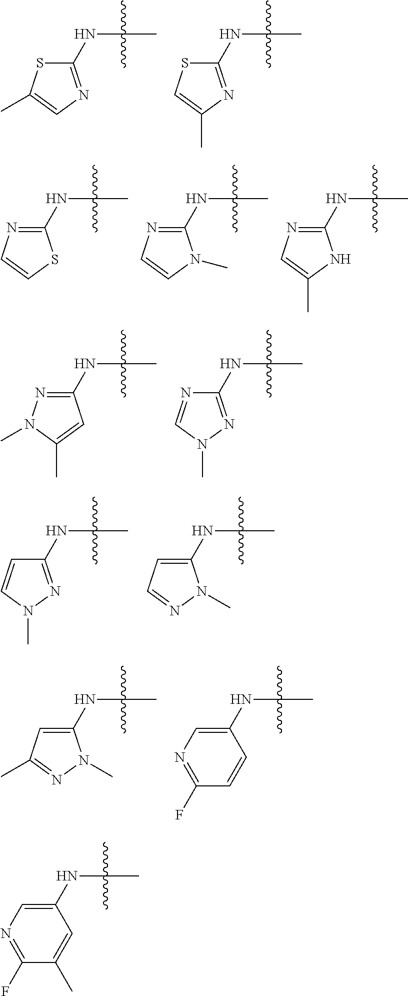

In an embodiment R³ or R⁵ is —NR^{a}—(C₃-C₈-cycloalkyl), which cycloalkyl is unsubstituted or substituted by C₁-C₆-alkyl or a C₁-C₃-alkylene bridge and R¹ is H or C₁-C₆-alkyl, for example:

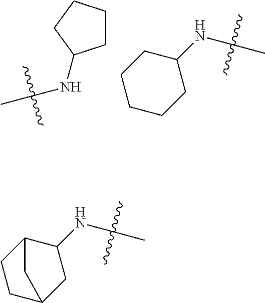

In an embodiment R³ or R⁵ is halo, for example Cl, F or Br.

In an embodiment R³ or R⁵ is —NR^{a}R^{b}, wherein R^{a} and R^{b} are independently selected from H and C₁-C₆-alkyl, for example —NH₂, —NHMe or —N(Me)₂.

In an embodiment R³ or R⁵ is hydroxy.

In an embodiment R³ or R⁵ is —NR^{a}(CO)O—C₁-C₆-alkyl, wherein R^{a} is H or C₁-C₆-alkyl, for example:

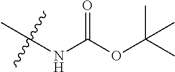 [SMILES: *NC(=O)OC(C)(C)C]

In an embodiment R³ or R⁵ is —O-(5 to 10 membered heteroaryl), which heteroaryl is unsubstituted or substituted by halo, C₁-C₆-alkyl, C₁-C₆-hydroxyalkyl, or —NR^{a}C(O) C₁-C₆-alkyl, for example:

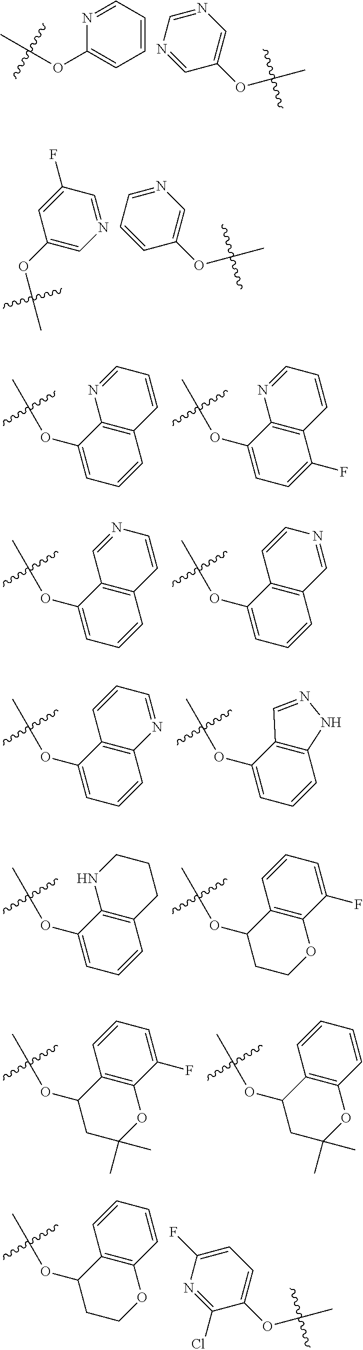

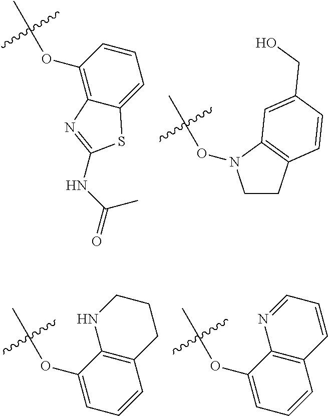

In an embodiment R³ or R⁵ is C₁-C₆-alkyl-R^{f} and R^{f} is aryl. In one embodiment, R^{f} is unsubstituted phenyl. In one embodiment, R^{f} is phenyl substituted by one or more substituent(s) selected from the group consisting of halo, C₁-C₆-alkoxy, C₁-C₆-haloalkyl, and C₁-C₆-hydroxyalkyl, for example:

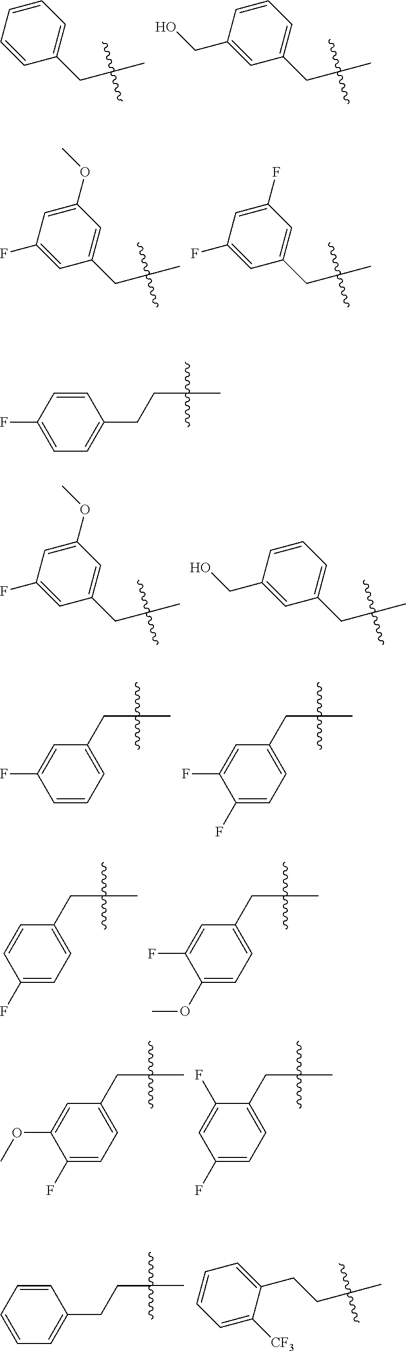

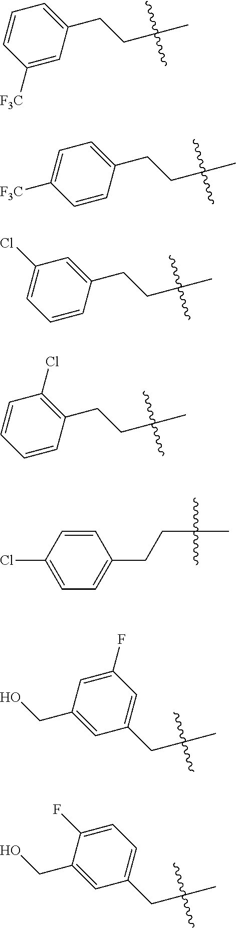

In an embodiment R³ or R⁵ is —C₁-C₆-alkoxy-R^{c}, wherein R^{c} is hydroxy, halo, C₁-C₆-alkoxy, C₁-C₆-alkenyl, phenyl unsubstituted or substituted by halo, 4 to 6 membered heterocycloalkyl unsubstituted or substituted by oxo or C₁-C₆-alkyl, 5 or 6 membered heteroaryl unsubstituted or substituted by C₁-C₆-alkyl, or C₃-C₈-cycloalkyl unsubstituted or substituted by halo or C₁-C₆-hydroxyalkyl, C₁-C₆-alkyl, for example:

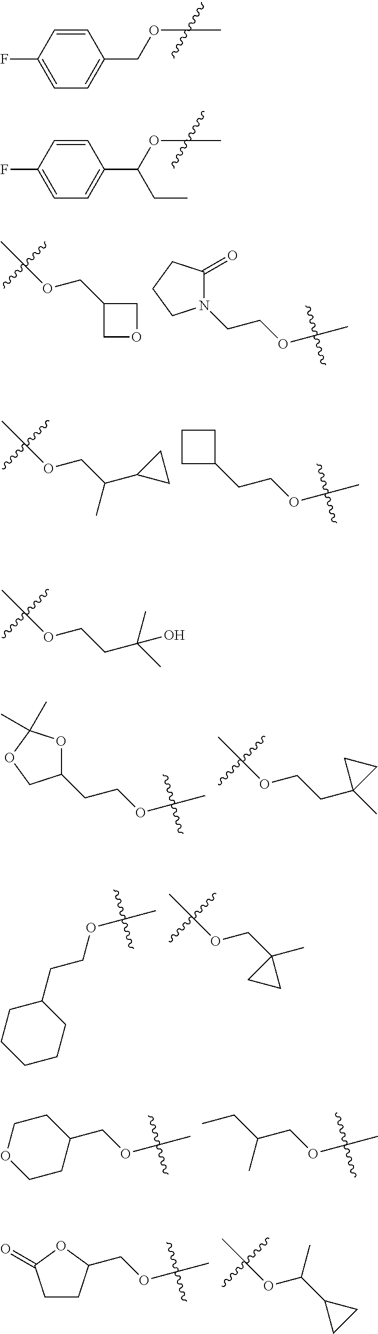

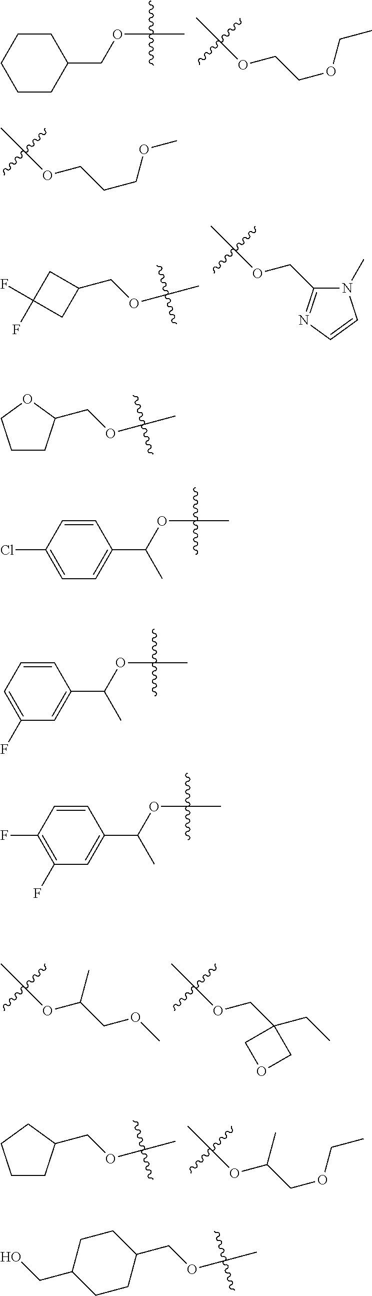

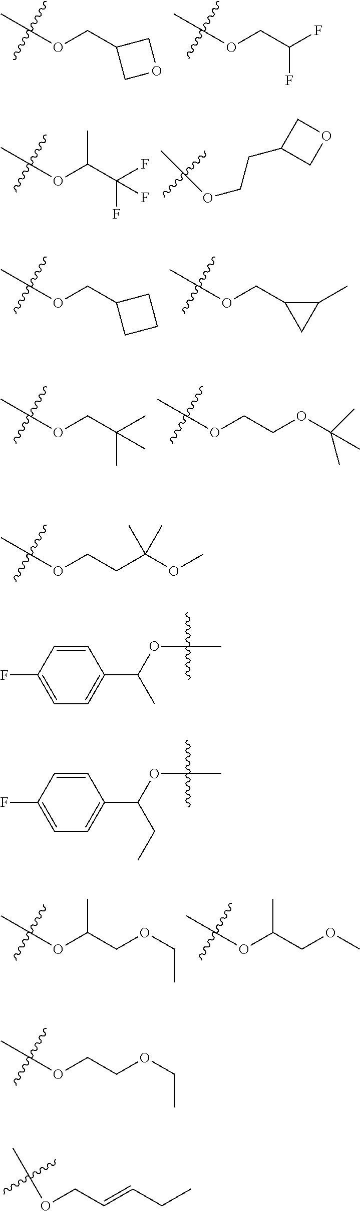

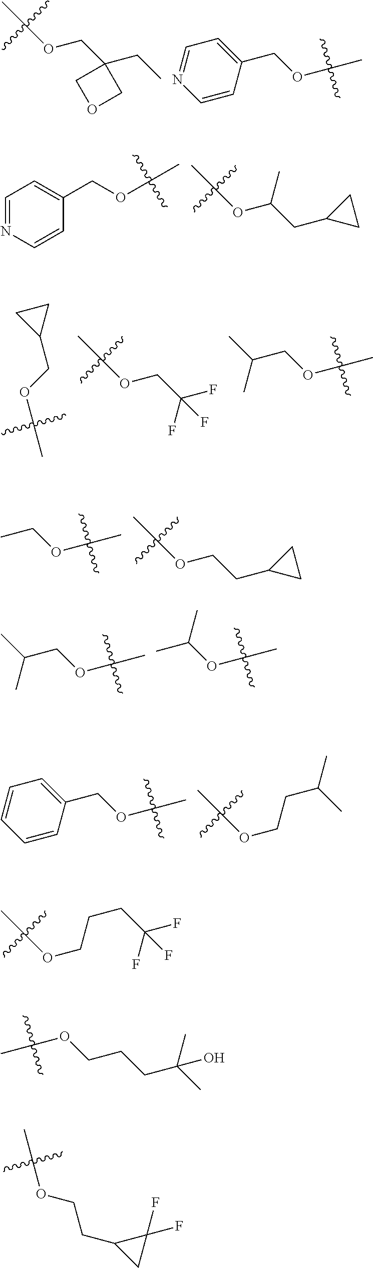

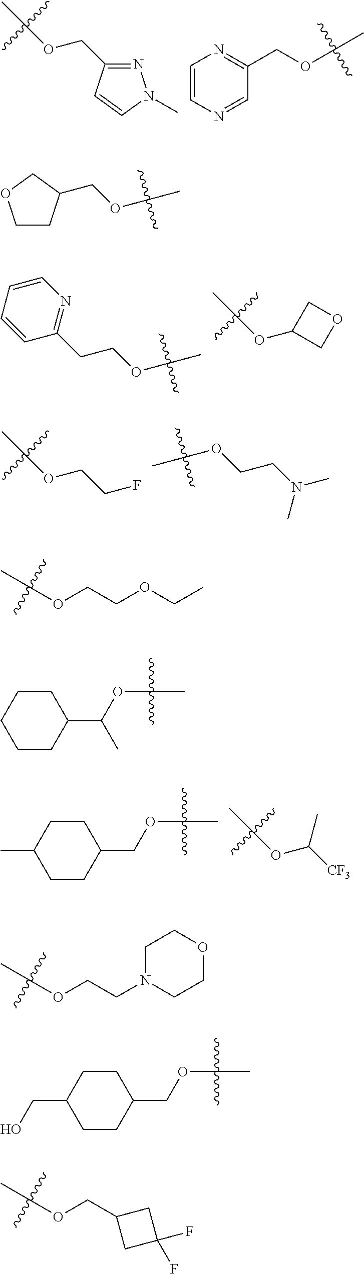

In an embodiment R³ or R⁵ is C₁-C₆-alkyl-R^{f} and R^{f} is 5 or 6 membered heterocycloalkyl, for example:

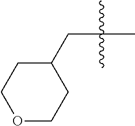 [SMILES: *CC1CCOCC1]

In an embodiment R³ or R⁵ is —O—C₃-C₆-cycloalkyl, which cycloalkyl is unsubstituted or substituted by halo, hydroxy, C₁-C₆-alkyl, phenyl, C₁-C₆-alkoxy, for example:

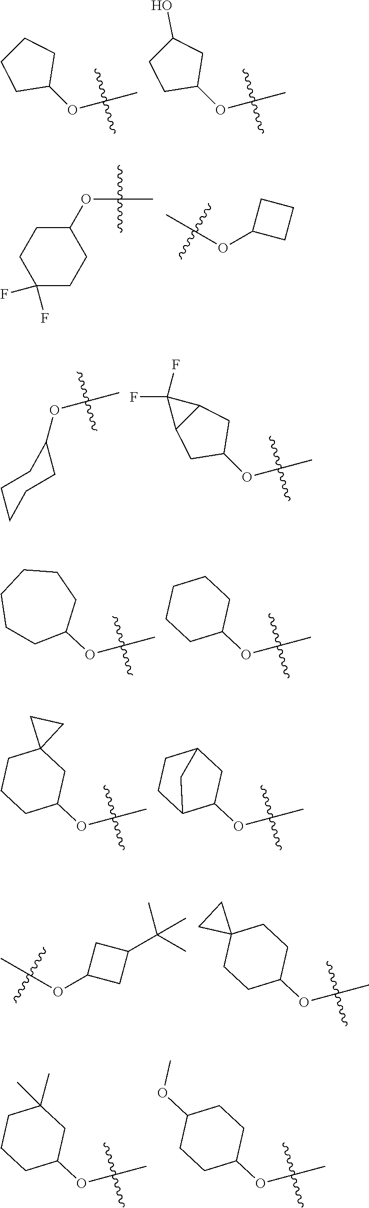

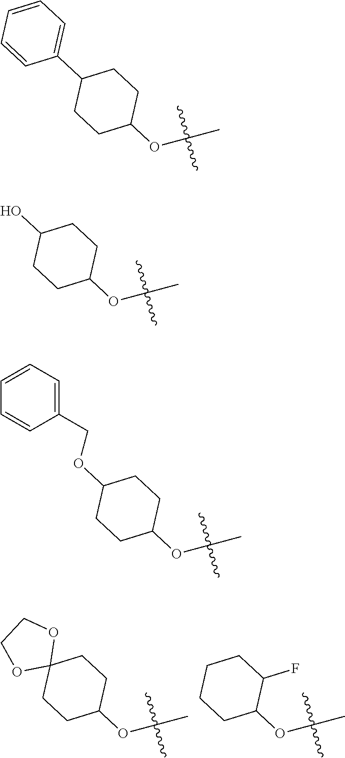 [SMILES: *OC1CCC(c2ccccc2)CC1 ; *OC1CCC(O)CC1 ; *OC1CCC(OCc2ccccc2)CC1 ; *OC1CCC2(CC1)OCCO2 ; *OC1CCCCC1F]

In an embodiment R³ or R⁵ is —O-(5 or 6 membered heterocycloalkyl), which heterocycloalkyl is unsubstituted or substituted by C₁-C₆-alkyl or —C(O)C₁-C₆-alkyl, for example:

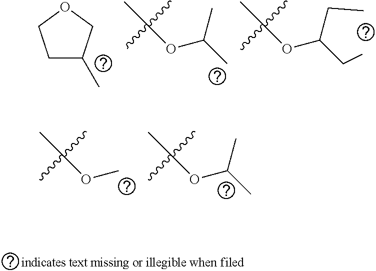

In an embodiment R³ or R⁵ is —NR^{a}—C₁-C₆-alkyl-R^{d}, wherein R^{d} is:

C₃-C₈-cycloalkyl, or phenyl unsubstituted or substituted by halo, for example:

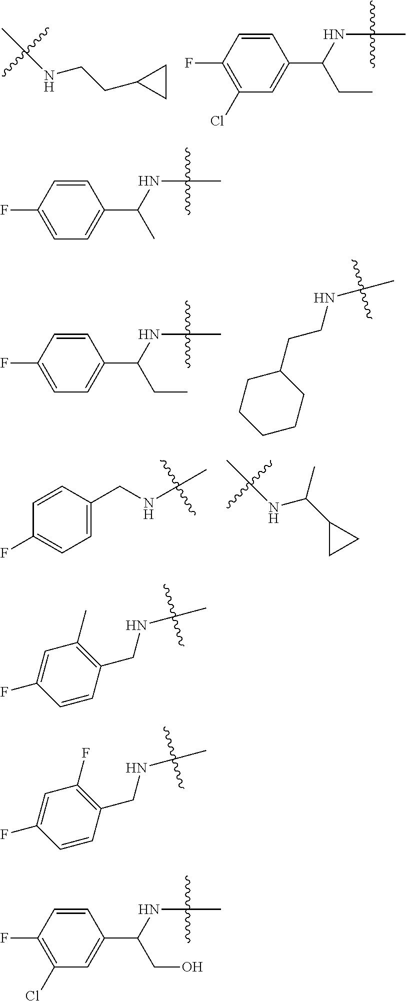

In an embodiment R³ or R⁵ is 5 to 10 membered heteroaryl unsubstituted or substituted by -hydroxy, NH₂, C₁-C₆-alkyl or C₁-C₆-hydroxyalkyl, for example:

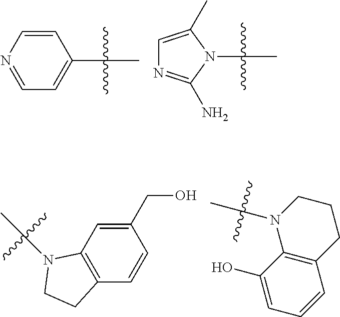

In an embodiment R³ or R⁵ is 5 or 6 membered heterocycloalkyl unsubstituted or substituted by halo, C₁-C₆-alkyl, —C(O)—C₃-C₈-cycloalkyl, oxo, 5 or 6 membered heterocycloalkyl, for example:

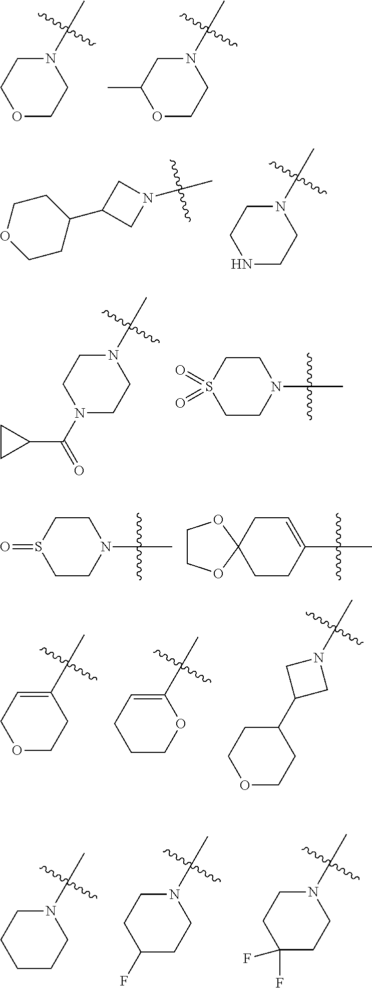

In an embodiment R³ or R⁵ is —C(O)NR^{a}—(C₁-C₆-alkyl)ₙ-R^{g}. In an embodiment R³ or R⁵ is —C(O)NR^{a}—(C₁-C₆-alkyl)-R‘ and R’ is C₃-C₆-cycloalkyl or phenyl, which phenyl is unsubstituted or substituted by halo or R³ or R⁵ is —C(O)NR^{a}—C₁-C₆-alkoxy, for example:

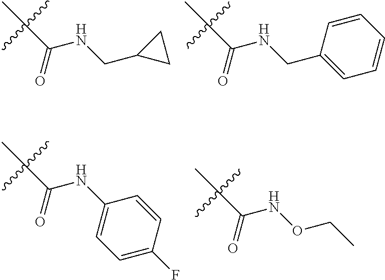

In an embodiment R³ or R⁵ is —S-(alkyl)ₙ-R^{h}. In one embodiment, R³ or R⁵ is —S-phenyl and said phenyl is unsubstituted or substituted by halo, for example:

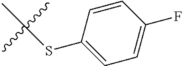 [SMILES: *Sc1ccc(F)cc1]

In an embodiment R³ or R⁵ is —C(O)—R^{e} and R^{e} is phenyl which phenyl is unsubstituted or substituted by halo, for example:

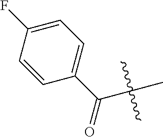 [SMILES: *C(=O)c1ccc(F)cc1]

In an embodiment R³ or R⁵ is —NR^{a}—S(O)₂-(4 to 6 membered heterocycloalkyl), for example:

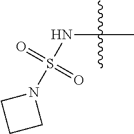 [SMILES: *NS(=O)(=O)N1CCC1]

In an embodiment R⁴ is H. In an embodiment R⁴ is halo. In an embodiment R⁴ is hydroxy. In an embodiment R⁴ is C₁-C₆-alkyl. In an embodiment R⁴ is C₁-C₆-haloalkyl. In an embodiment R⁴ is C₁-C₆-hydroxalkyl. In an embodiment R⁴ is CN. In an embodiment R⁴ is C₁-C₆-alkoxy unsubstituted or substituted by hydroxy or C₁-C₆-alkoxy. In an embodiment R⁴ is —(C₁-C₆-alkyl)ₙ-(C₃-C₈-cycloalkyl). In an embodiment R⁴ is —(C₁-C₆-alkyl)ₙ-(C₃-C₈-cycloalkenyl). In an embodiment R⁴ is —(C₁-C₆-alkyl)ₙ-(4 to 10 membered heterocycloalkyl) unsubstituted or substituted by one or more substituent(s) selected from the group consisting of halo, C₁-C₆-alkyl, or —C(O)—C₁-C₆-alkyl.

In an embodiment R⁴ is —NR^{a}R^{b} and R^{a} and R^{b} are as defined herein, for example:

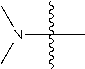 [SMILES: *N(C)C]

In an embodiment R⁴ is C₁-C₆-alkoxy unsubstituted or substituted by hydroxy, C₁-C₆-alkoxy or —NR^{a}R^{b}, wherein R^{a} and R^{b} are as defined herein, for example:

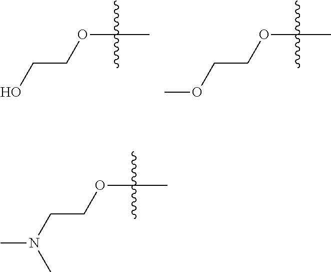

In an embodiment R⁴ is C₃-C₆-cycloalkyl or C₃-C₆-cycloalkenyl, for example:

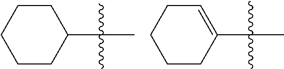 [SMILES: *C1CCCCC1 ; *C1=CCCCC1]

In an embodiment R⁴ is 4 to 10 membered heterocycloalkyl unsubstituted or substituted by halo, hydroxy, cyano, oxo, C₁-C₆-alkyl, C₁-C₆-alkoxy, C₁-C₆-hydroxyalkyl, —C(O)OH, —C(O)—C₁-C₆-alkyl, —C(O)—C₃-C₈-cycloalkyl, —C(O)-phenyl, 4 to 10 membered heterocycloalkyl, —C(O)(5 or 6 membered heteroaryl), —C(O)(4 to 10 membered heterocycloalkyl), C₁-C₄-alkylene bridge, for example:

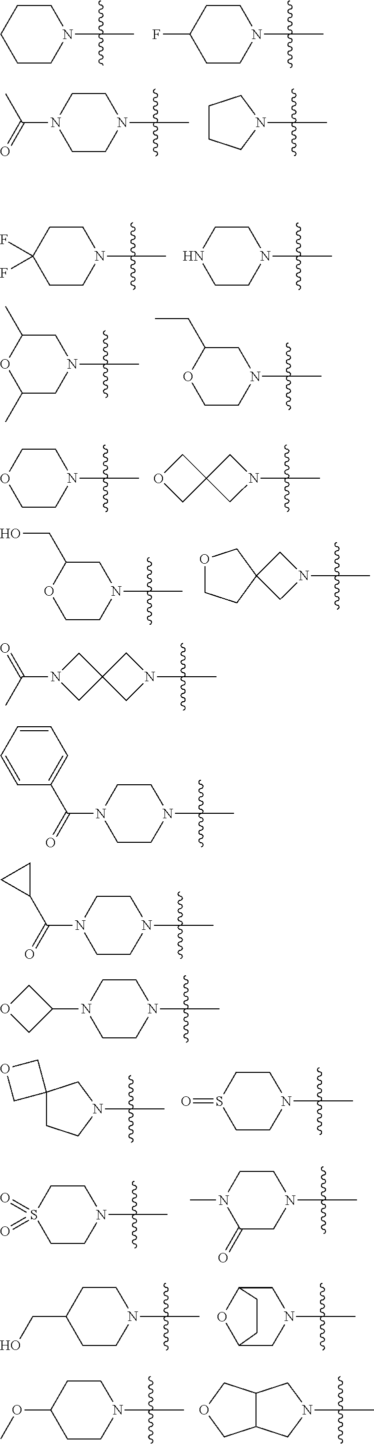

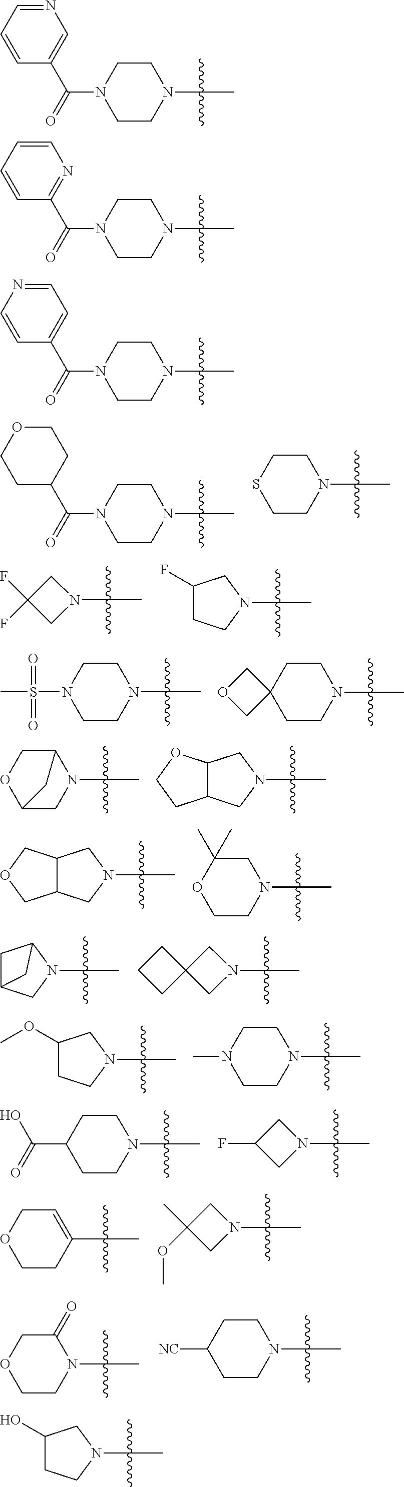

In an embodiment of the present invention R⁷ is 5 or 6 membered heteroaryl which is unsubstituted or substituted by one or more substituent(s) selected from the group consisting of halo, C₁-C₆-alkyl, C₁-C₆-alkoxy or —O-aryl, —S-aryl, —NH-aryl, —(C₁-C₆-alkyl)-aryl, for example.

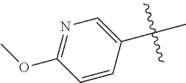 [SMILES: *c1ccc(OC)nc1]

In an embodiment of the present invention R⁸ is OH. In an embodiment of the present invention R⁸ is —NH₂. In an embodiment of the present invention R⁸ is C₁-C₆-alkoxy. In an embodiment of the present invention R⁸ is —C(O)O—C₁-C₆-alkyl.

In an embodiment of the present invention or R⁶ and R⁷ together with the carbon atoms to which they are attached form a 5 membered ring selected from a cycloalkyl or heterocycloalkyl having 5 ring members, so that the compounds of Formula (I) are as following:

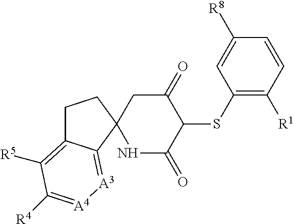

In an embodiment of the present invention R² and R³ together with the atoms to which they are attached form a naphthyl or 9 or 10 membered heteroaryl, each of which is unsubstituted or substituted by one or more substituent(s) selected from the group consisting of halo, hydroxy, —NH₂, —NH(C₁-C₆-alkyl), —N(C₁-C₆-alkyl)₂, C₁-C₆-alkyl, C₁-C₆-alkoxy and C₁-C₆-haloalkyl.

In an embodiment of the present invention R³ and R⁴ together with the atoms to which they are attached form a naphthyl or 9 or 10 membered heteroaryl, each of which is unsubstituted or substituted by one or more substituent(s) selected from the group consisting of halo, hydroxy, —NH₂, —NH(C₁-C₆-alkyl), —N(C₁-C₆-alkyl)₂, C₁-C₆-alkyl, C₁-C₆-alkoxy and C₁-C₆-haloalkyl.

In an embodiment of the present invention R⁵ and R⁶ together with the atoms to which they are attached form a naphthyl or 9 or 10 membered heteroaryl, each of which is unsubstituted or substituted by one or more substituent(s) selected from the group consisting of halo, hydroxy, —NH₂, —NH(C₁-C₆-alkyl), —N(C₁-C₆-alkyl)₂, C₁-C₆-alkyl, C₁-C₆-alkoxy and C₁-C₆-haloalkyl.

In an embodiment of the present invention n is 0. In an embodiment of the present invention n is 1.

In an embodiment R⁹ is H. In an embodiment R⁹ is C₁-C₆-alkyl. In an embodiment R⁹ is C₃-C₈-cycloalkyl. In an embodiment R⁹ is halo. In an embodiment R⁹ is —O-aryl, for example —O-phenyl.

In an embodiment R⁹ is —S-aryl, for example —S-phenyl. In an embodiment R⁹ is —NH-aryl, for example —NH-phenyl. In an embodiment R⁹ is —(C₁-C₆-alkyl)ₙ-aryl, for example —(C₁-C₆-alkyl)ₙ-phenyl.

In an embodiment R¹⁰ is H. In an embodiment R¹⁰ is C₁-C₆-alkyl. In an embodiment R¹⁰ is C₃-C₈-cycloalkyl. In an embodiment R¹⁰ is halo. In an embodiment R¹⁰ is —O-aryl, for example —O-phenyl. In an embodiment R¹⁰ is —S-aryl, for example —S-phenyl. In an embodiment R¹⁰ is —NH-aryl, for example —NH-phenyl. In an embodiment R¹⁰ is —(C₁-C₆-alkyl)-aryl, for example —(C₁-C₆-alkyl)-phenyl.

In one embodiment A³ is NH. In one embodiment A³ is CR², wherein R² is selected from the group consisting of H, halo, hydroxy, C₁-C₆-hydroxyalkyl, and NH. In one embodiment, R⁹ and R¹⁰ are H. In one embodiment R¹ is Cl. In one embodiment R³ is NH-phenyl or NH-pyridinyl, which phenyl or pyridinyl is substituted by halo. In one embodiment R⁴, R⁵, R⁶ and R⁸ are H.

In an embodiment A¹ is O, A² is NH, R¹ is Cl, A³ is NH, A⁴ is CR³ and R³ is NH-phenyl or NH-pyridinyl, which phenyl or pyridinyl is substituted by halo, R⁴, R⁵ and R⁶ are H, R⁷ is thiophenyl.

In an embodiment A¹ is S, A² is NH, R¹ is halo, A³ is NH, A⁴ is CR³ and R³ is NH-phenyl or NH-pyridinyl, which phenyl or pyridinyl is substituted by halo, R⁴, R⁵ and R⁶ are H, R⁷ is thiophenyl.

In an embodiment, the compound of Formula (I) is selected from the compounds of the following compounds and stereoisomers, tautomers, and pharmaceutically acceptable salts thereof.

These compounds can also be prepared as a racemate, mixture of diastereisomer or as single stereoisomers, all of which forms fall within the scope of the invention:
1-[4-[5-(2-chlorophenyl)sulfanyl-4,6-dioxo-2-(3-thienyl)-2-piperidyl]phenyl]piperidine-4-carbonitrile;
2-[[6-(6-bromo-2-pyridyl)-2,4-dioxo-6-(3-thienyl)-3-piperidyl]sulfanyl]benzonitrile;
3-(2-chloro-5-hydroxy-phenyl)sulfanyl-6-[4-(1-piperidyl)phenyl]-6-(3-thienyl)piperidine-2,4-dione;
3-(2-chlorophenoxy)-6-(4-morpholinophenyl)-6-(3-thienyl)piperidine-2,4-dione;
3-(2-chlorophenoxy)-6-[4-(1-piperidyl)phenyl]-6-(3-thienyl)piperidine-2,4-dione;
3-(2-chlorophenoxy)-6-[6-(2-cyclopropylethoxy)-2-pyridyl]-6-(3-thienyl)piperidine-2,4-dione;
3-(2-chlorophenoxy)-6-[6-(3,4-difluorophenoxy)-2-pyridyl]-6-(3-thienyl)piperidine-2,4-dione;
3-(2-chlorophenoxy)-6-[6-(4-fluoroanilino)-2-pyridyl]-6-(3-thienyl)piperidine-2,4-dione;
3-(2-chlorophenoxy)-6-[6-(4-fluorophenoxy)-2-pyridyl]-6-(3-thienyl)piperidine-2,4-dione;
3-(2-chlorophenyl)sulfanyl-1-methyl-6-(3-tetrahydropyran-4-yloxyphenyl)-6-(3-thienyl)piperidine-2,4-dione;
3-(2-chlorophenyl)sulfanyl-1-methyl-6-[3-(tetrahydropyran-4-ylamino)phenyl]-6-(3-thienyl)piperidine-2,4-dione;
3-(2-chlorophenyl)sulfanyl-6-(1H-indol-4-yl)-6-(3-thienyl)piperidine-2,4-dione;
3-(2-chlorophenyl)sulfanyl-6-(2-fluorophenyl)-1-methyl-6-(3-thienyl)piperidine-2,4-dione;
3-(2-chlorophenyl)sulfanyl-6-(2-hydroxy-4-morpholino-phenyl)-6-(3-thienyl)piperidine-2,4-dione;
3-(2-chlorophenyl)sulfanyl-6-(2-hydroxyphenyl)-6-(3-thienyl)piperidine-2,4-dione;
3-(2-chlorophenyl)sulfanyl-6-(2-naphthyl)-6-(3-thienyl)piperidine-2,4-dione;
3-(2-chlorophenyl)sulfanyl-6-(3-fluoro-4-morpholino-phenyl)-6-(3-thienyl)piperidine-2,4-dione;
3-(2-chlorophenyl)sulfanyl-6-(3-hydroxyphenyl)-6-(3-thienyl)piperidine-2,4-dione;
3-(2-chlorophenyl)sulfanyl-6-(3-tetrahydropyran-4-yloxyphenyl)-6-(3-thienyl)piperidine-2,4-dione;
3-(2-chlorophenyl)sulfanyl-6-(3-thienyl)-6-(4-thiomorpholinophenyl)piperidine-2,4-dione;
3-(2-chlorophenyl)sulfanyl-6-(3-thienyl)-6-[6-(2,2,2-trifluoro-1-methyl-ethoxy)-2-pyridyl]piperidine-2,4-dione;
3-(2-chlorophenyl)sulfanyl-6-(3-thienyl)-6-[6-(2,2,2-trifluoroethoxy)-2-pyridyl]piperidine-2,4-dione;
3-(2-chlorophenyl)sulfanyl-6-(3-thienyl)-6-[6-(4,4,4-trifluorobutoxy)-2-pyridyl]piperidine-2,4-dione;
3-(2-chlorophenyl)sulfanyl-6-(3-thienyl)-6-[6-[3-(trifluoromethyl)phenoxy]-2-pyridyl]piperidine-2,4-dione;
3-(2-chlorophenyl)sulfanyl-6-(3-thienyl)-6-[6-[4-(trifluoromethoxy)phenoxy]-2-pyridyl]piperidine-2,4-dione;
3-(2-chlorophenyl)sulfanyl-6-(3-thienyl)-6-[6-[4-(trifluoromethyl)cyclohexoxy]-2-pyridyl]piperidine-2,4-dione;
3-(2-chlorophenyl)sulfanyl-6-(3-thienyl)-6-[6-[4-(trifluoromethyl)phenoxy]-2-pyridyl]piperidine-2,4-dione;
3-(2-chlorophenyl)sulfanyl-6-(4-cyclohexylphenyl)-6-(3-thienyl)piperidine-2,4-dione;
3-(2-chlorophenyl)sulfanyl-6-(4-cyclopropylphenyl)-6-(3-thienyl)piperidine-2,4-dione;
3-(2-chlorophenyl)sulfanyl-6-(4-hydroxyphenyl)-6-(3-thienyl)piperidine-2,4-dione;
3-(2-chlorophenyl)sulfanyl-6-(4-morpholino-3-phenyl-phenyl)-6-(3-thienyl)piperidine-2,4-dione;
3-(2-chlorophenyl)sulfanyl-6-(4-morpholinophenyl)-6-(3-thienyl)piperidine-2,4-dione;
3-(2-chlorophenyl)sulfanyl-6-(4-morpholinophenyl)-6-(5-phenyl-3-thienyl)piperidine-2,4-dione;
3-(2-chlorophenyl)sulfanyl-6-(4-morpholinophenyl)-6-(6-tetrahydropyran-4-yloxy-2-pyridyl)piperidine-2,4-dione;
3-(2-chlorophenyl)sulfanyl-6-(4-morpholinophenyl)-6-thiazol-4-yl-piperidine-2,4-dione;
3-(2-chlorophenyl)sulfanyl-6-(4-piperazin-1-ylphenyl)-6-(3-thienyl)piperidine-2,4-dione;
3-(2-chlorophenyl)sulfanyl-6-(4-pyrrolidin-1-ylphenyl)-6-(3-thienyl)piperidine-2,4-dione;
3-(2-chlorophenyl)sulfanyl-6-(5-chloro-3-thienyl)-6-[6-(4-fluorophenoxy)-2-pyridyl]piperidine-2,4-dione;
3-(2-chlorophenyl)sulfanyl-6-(5-methyl-3-thienyl)-6-(4-morpholinophenyl)piperidine-2,4-dione;
3-(2-chlorophenyl)sulfanyl-6-(6-chroman-4-yloxy-2-pyridyl)-6-(3-thienyl)piperidine-2,4-dione;
3-(2-chlorophenyl)sulfanyl-6-(6-ethoxy-2-pyridyl)-6-(3-thienyl)piperidine-2,4-dione;
3-(2-chlorophenyl)sulfanyl-6-(6-indan-5-yloxy-2-pyridyl)-6-(3-thienyl)piperidine-2,4-dione;
3-(2-chlorophenyl)sulfanyl-6-(6-isobutoxy-2-pyridyl)-6-(3-thienyl)piperidine-2,4-dione;
3-(2-chlorophenyl)sulfanyl-6-(6-isopentyloxy-2-pyridyl)-6-(3-thienyl)piperidine-2,4-dione;
3-(2-chlorophenyl)sulfanyl-6-(6-isopropoxy-2-pyridyl)-6-(3-thienyl)piperidine-2,4-dione;
3-(2-chlorophenyl)sulfanyl-6-(6-isopropoxy-5-morpholino-2-pyridyl)-6-(3-thienyl)piperidine-2,4-dione
3-(2-chlorophenyl)sulfanyl-6-(6-morpholino-3-pyridyl)-6-(3-thienyl)piperidine-2,4-dione;
3-(2-chlorophenyl)sulfanyl-6-(6-pent-2-enoxy-2-pyridyl)-6-(3-thienyl)piperidine-2,4-dione;
3-(2-chlorophenyl)sulfanyl-6-(6-phenoxy-2-pyridyl)-6-(3-thienyl)piperidine-2,4-dione;
3-(2-chlorophenyl)sulfanyl-6-(6-phenyl-2-pyridyl)-6-(3-thienyl)piperidine-2,4-dione;
3-(2-chlorophenyl)sulfanyl-6-(6-pyrimidin-5-yloxy-2-pyridyl)-6-(3-thienyl)piperidine-2,4-dione;
3-(2-chlorophenyl)sulfanyl-6-(6-tetrahydrofuran-3-yloxy-2-pyridyl)-6-(3-thienyl)piperidine-2,4-dione;
3-(2-chlorophenyl)sulfanyl-6-(6-tetralin-1-yloxy-2-pyridyl)-6-(3-thienyl)piperidine-2,4-dione;
3-(2-chlorophenyl)sulfanyl-6-[3-(4-fluoroanilino)phenyl]-1-methyl-6-(3-thienyl)piperidine-2,4-dione;
3-(2-chlorophenyl)sulfanyl-6-[3-(4-fluoroanilino)phenyl]-6-(3-thienyl)piperidine-2,4-dione;
3-(2-chlorophenyl)sulfanyl-6-[3-(4-fluoroanilino)phenyl]-6-phenyl-piperidine-2,4-dione;
3-(2-chlorophenyl)sulfanyl-6-[3-(4-fluoro-N-methyl-anilino)phenyl]-6-phenyl-piperidine-2,4-dione;
3-(2-chlorophenyl)sulfanyl-6-[3-(4-fluorophenoxy)phenyl]-6-(3-thienyl)piperidine-2,4-dione;
3-(2-chlorophenyl)sulfanyl-6-[3-(cyclohexylamino)phenyl]-6-(3-thienyl)piperidine-2,4-dione;
3-(2-chlorophenyl)sulfanyl-6-[3-(tetrahydropyran-4-yl amino)phenyl]-6-(3-thienyl)piperidine-2,4-dione
3-(2-chlorophenyl)sulfanyl-6-[3-[(6-fluoro-5-methyl-3-pyridyl)amino]phenyl]-6-(3-thienyl)piperidine-2,4-dione;
3-(2-chlorophenyl)sulfanyl-6-[4-(1-piperidyl)phenyl]-6-(3-thienyl)piperidine-2,4-dione;
3-(2-chlorophenyl)sulfanyl-6-[4-(2,2-dimethylmorpholin-4-yl)phenyl]-6-(3-thienyl)piperidine-2,4-dione;
3-(2-chlorophenyl)sulfanyl-6-[4-(2,6-dimethylmorpholin-4-yl)phenyl]-6-(3-thienyl)piperidine-2,4-dione;
3-(2-chlorophenyl)sulfanyl-6-[4-(2-ethylmorpholin-4-yl)phenyl]-6-(3-thienyl)piperidine-2,4-dione;
3-(2-chlorophenyl)sulfanyl-6-[4-(2-hydroxyethoxy)phenyl]-6-(3-thienyl)piperidine-2,4-dione;
3-(2-chlorophenyl)sulfanyl-6-[4-(2-methoxyethoxy)phenyl]-6-(3-thienyl)piperidine-2,4-dione;
3-(2-chlorophenyl)sulfanyl-6-[4-(2-methylmorpholin-4-yl)phenyl]-6-(3-thienyl)piperidine-2,4-dione;
3-(2-chlorophenyl)sulfanyl-6-[4-(2-oxa-5-azabicyclo[2.2.1]heptan-5-yl)phenyl]-6-(3-thienyl)piperidine-2,4-dione;
3-(2-chlorophenyl)sulfanyl-6-[4-(2-oxa-6-azaspiro[3.3]heptan-6-yl)phenyl]-6-(3-thienyl)piperidine-2,4-dione;
3-(2-chlorophenyl)sulfanyl-6-[4-(2-oxa-7-azaspiro[3.5]nonan-7-yl)phenyl]-6-(3-thienyl)piperidine-2,4-dione;
3-(2-chlorophenyl)sulfanyl-6-[4-(3,3-difluoroazetidin-1-yl)phenyl]-6-(3-thienyl)piperidine-2,4-dione;
3-(2-chlorophenyl)sulfanyl-6-[4-(3,3-difluoropyrrolidin-1-yl)phenyl]-6-(3-thienyl)piperidine-2,4-dione
3-(2-chlorophenyl)sulfanyl-6-[4-(3-fluoroazetidin-1-yl)phenyl]-6-(3-thienyl)piperidine-2,4-dione;
3-(2-chlorophenyl)sulfanyl-6-[4-(3-fluoropyrrolidin-1-yl)phenyl]-6-(3-thienyl)piperidine-2,4-dione;
3-(2-chlorophenyl)sulfanyl-6-[4-(3-hydroxypropoxy)phenyl]-6-(3-thienyl)piperidine-2,4-dione;
3-(2-chlorophenyl)sulfanyl-6-[4-(3-methoxypropoxy)phenyl]-6-(3-thienyl)piperidine-2,4-dione;
3-(2-chlorophenyl)sulfanyl-6-[4-(3-methoxypyrrolidin-1-yl)phenyl]-6-(3-thienyl)piperidine-2,4-dione;
3-(2-chlorophenyl)sulfanyl-6-[4-(4,4-difluoro-1-piperidyl)phenyl]-6-(3-thienyl)piperidine-2,4-dione;
3-(2-chlorophenyl)sulfanyl-6-[4-(4-fluoro-1-piperidyl)phenyl]-6-(3-thienyl)piperidine-2,4-dione;
3-(2-chlorophenyl)sulfanyl-6-[4-(4-methoxy-1-piperidyl)phenyl]-6-(3-thienyl)piperidine-2,4-dione;
3-(2-chlorophenyl)sulfanyl-6-[4-(8-oxa-3-azabicyclo[3.2.1]octan-3-yl)phenyl]-6-(3-thienyl)piperidine-2,4-dione;
3-(2-chlorophenyl)sulfanyl-6-[4-(cyclohexen-1-yl)phenyl]-6-(3-thienyl)piperidine-2,4-dione;
3-(2-chlorophenyl)sulfanyl-6-[4-(dimethylamino)phenyl]-6-(3-thienyl)piperidine-2,4-dione;
3-(2-chlorophenyl)sulfanyl-6-[4-(tetrahydropyran-4-yl amino)phenyl]-6-(3-thienyl)piperidine-2,4-dione
3-(2-chlorophenyl)sulfanyl-6-[5-(4-fluoroanilino)-2-hydroxy-phenyl]-6-(3-thienyl)piperidine-2,4-dione
3-(2-chlorophenyl)sulfanyl-6-[5-[(4-fluorophenyl)methyl]-3-thienyl]-6-(4-morpholinophenyl)piperidine-2,4-dione;
3-(2-chlorophenyl)sulfanyl-6-[6-(1,2,3,4-tetrahydroquinolin-8-yloxy)-2-pyridyl]-6-(3-thienyl)piperidine-2,4-dione;
3-(2-chlorophenyl)sulfanyl-6-[6-(1-cyclohexylethoxy)-2-pyridyl]-6-(3-thienyl)piperidine-2,4-dione;
3-(2-chlorophenyl)sulfanyl-6-[6-(1-cyclopropylethoxy)-2-pyridyl]-6-(3-thienyl)piperidine-2,4-dione;
3-(2-chlorophenyl)sulfanyl-6-[6-(1-cyclopropylethylamino)-2-pyridyl]-6-(3-thienyl)piperidine-2,4-dione;
3-(2-chlorophenyl)sulfanyl-6-[6-(1H-indazol-4-yloxy)-2-pyridyl]-6-(3-thienyl)piperidine-2,4-dione;
3-(2-chlorophenyl)sulfanyl-6-[6-(2,2-difluoroethoxy)-2-pyridyl]-6-(3-thienyl)piperidine-2,4-dione;
3-(2-chlorophenyl)sulfanyl-6-[6-(2,2-dimethylchroman-4-yl)oxy-2-pyridyl]-6-(3-thienyl)piperidine-2,4-dione;
3-(2-chlorophenyl)sulfanyl-6-[6-(2,2-dimethylpropoxy)-2-pyridyl]-6-(3-thienyl)piperidine-2,4-dione;
3-(2-chlorophenyl)sulfanyl-6-[6-(2,3-difluorophenoxy)-2-pyridyl]-6-(3-thienyl)piperidine-2,4-dione;
3-(2-chlorophenyl)sulfanyl-6-[6-(2,4-difluorophenoxy)-2-pyridyl]-6-(3-thienyl)piperidine-2,4-dione;
3-(2-chlorophenyl)sulfanyl-6-[6-(2-cyclobutylethoxy)-2-pyridyl]-6-(3-thienyl)piperidine-2,4-dione;
3-(2-chlorophenyl)sulfanyl-6-[6-(2-cyclohexylethoxy)-2-pyridyl]-6-(3-thienyl)piperidine-2,4-dione;
3-(2-chlorophenyl)sulfanyl-6-[6-(2-cyclohexylethylamino)-2-pyridyl]-6-(3-thienyl)piperidine-2,4-dione;
3-(2-chlorophenyl)sulfanyl-6-[6-(2-cyclopentylethoxy)-2-pyridyl]-6-(3-thienyl)piperidine-2,4-dione;
3-(2-chlorophenyl)sulfanyl-6-[6-(2-cyclopropyl-1-methyl-ethoxy)-2-pyridyl]-6-(3-thienyl)piperidine-2,4-dione;
3-(2-chlorophenyl)sulfanyl-6-[6-(2-cyclopropylethoxy)-2-pyridyl]-6-(3-thienyl)piperidine-2,4-dione;
3-(2-chlorophenyl)sulfanyl-6-[6-(2-cyclopropylethylamino)-2-pyridyl]-6-(3-thienyl)piperidine-2,4-dione;
3-(2-chlorophenyl)sulfanyl-6-[6-(2-cyclopropylpropoxy)-2-pyridyl]-6-(3-thienyl)piperidine-2,4-dione;
3-(2-chlorophenyl)sulfanyl-6-[6-(2-ethoxy-1-methyl-ethoxy)-2-pyridyl]-6-(3-thienyl)piperidine-2,4-dione;
3-(2-chlorophenyl)sulfanyl-6-[6-(2-ethoxyethoxy)-2-pyridyl]-6-(3-thienyl)piperidine-2,4-dione;
3-(2-chlorophenyl)sulfanyl-6-[6-(2-fluorophenoxy)-2-pyridyl]-6-(3-thienyl)piperidine-2,4-dione;
3-(2-chlorophenyl)sulfanyl-6-[6-(2-methoxy-1-methyl-ethoxy)-2-pyridyl]-6-(3-thienyl)piperidine-2,4-dione;
3-(2-chlorophenyl)sulfanyl-6-[6-(2-methoxyphenoxy)-2-pyridyl]-6-(3-thienyl)piperidine-2,4-dione;
3-(2-chlorophenyl)sulfanyl-6-[6-(2-methylbutoxy)-2-pyridyl]-6-(3-thienyl)piperidine-2,4-dione;
3-(2-chlorophenyl)sulfanyl-6-[6-(2-morpholino-4-pyridyl)-2-pyridyl]-6-(3-thienyl)piperidine-2,4-dione
3-(2-chlorophenyl)sulfanyl-6-[6-(2-pyridyloxy)-2-pyridyl]-6-(3-thienyl)piperidine-2,4-dione;
3-(2-chlorophenyl)sulfanyl-6-[6-(3,4-difluoroanilino)-2-pyridyl]-6-(3-thienyl)piperidine-2,4-dione;
3-(2-chlorophenyl)sulfanyl-6-[6-(3,4-difluorophenoxy)-2-pyridyl]-6-(3-thienyl)piperidine-2,4-dione;
3-(2-chlorophenyl)sulfanyl-6-[6-(3,4-difluorophenoxy)-2-pyridyl]-6-(4-morpholinophenyl)piperidine-2,4-dione;
3-(2-chlorophenyl)sulfanyl-6-[6-(3,4-difluorophenoxy)-2-pyridyl]-6-[4-(1-piperidyl)phenyl]piperidine-2,4-dione;
3-(2-chlorophenyl)sulfanyl-6-[6-(3,5-difluorophenoxy)-2-pyridyl]-6-(3-thienyl)piperidine-2,4-dione;
3-(2-chlorophenyl)sulfanyl-6-[6-(3-fluoro-4-methoxy-phenoxy)-2-pyridyl]-6-(3-thienyl)piperidine-2,4-dione;
3-(2-chlorophenyl)sulfanyl-6-[6-(3-fluorophenoxy)-2-pyridyl]-6-(3-thienyl)piperidine-2,4-dione;
3-(2-chlorophenyl)sulfanyl-6-[6-(3-hydroxy-3-methyl-butoxy)-2-pyridyl]-6-(3-thienyl)piperidine-2,4-dione;
3-(2-chlorophenyl)sulfanyl-6-[6-(3-hydroxycyclopentoxy)-2-pyridyl]-6-(3-thienyl)piperidine-2,4-dione
3-(2-chlorophenyl)sulfanyl-6-[6-(3-methoxy-3-methyl-butoxy)-2-pyridyl]-6-(3-thienyl)piperidine-2,4-dione;
3-(2-chlorophenyl)sulfanyl-6-[6-(3-methoxy-N-methyl-anilino)-2-pyridyl]-6-(3-thienyl)piperidine-2,4-dione;
3-(2-chlorophenyl)sulfanyl-6-[6-(3-methoxyphenoxy)-2-pyridyl]-6-(3-thienyl)piperidine-2,4-dione;
3-(2-chlorophenyl)sulfanyl-6-[6-(3-methoxypropoxy)-2-pyridyl]-6-(3-thienyl)piperidine-2,4-dione;
3-(2-chlorophenyl)sulfanyl-6-[6-(3-pyridyloxy)-2-pyridyl]-6-(3-thienyl)piperidine-2,4-dione;
3-(2-chlorophenyl)sulfanyl-6-[6-(3-tetrahydropyran-4-ylazetidin-1-yl)-2-pyridyl]-6-(3-thienyl)piperidine-2,4-dione;
3-(2-chlorophenyl)sulfanyl-6-[6-(4,4-difluorocyclohexoxy)-2-pyridyl]-6-(3-thienyl)piperidine-2,4-dione;
3-(2-chlorophenyl)sulfanyl-6-[6-(4-cyclopropyl-2-fluoro-anilino)-2-pyridyl]-6-(3-thienyl)piperidine-2,4-dione;
3-(2-chlorophenyl)sulfanyl-6-[6-(4-fluoro-2-isopropyl-phenoxy)-2-pyridyl]-6-(3-thienyl)piperidine-2,4-dione;
3-(2-chlorophenyl)sulfanyl-6-[6-(4-fluoro-2-methoxy-phenoxy)-2-pyridyl]-6-(3-thienyl)piperidine-2,4-dione;
(6S)-3-(2-chlorophenyl)sulfanyl-6-[6-(4-fluoro-2-methoxy-phenoxy)-2-pyridyl]-6-(3-thienyl)piperidine-2,4-dione;
3-(2-chlorophenyl)sulfanyl-6-[6-(4-fluoro-2-tetrahydropyran-4-yl-phenoxy)-2-pyridyl]-6-(3-thienyl)piperidine-2,4-dione;
3-(2-chlorophenyl)sulfanyl-6-[6-(4-fluoro-3-methoxy-phenyl)-2-pyridyl]-6-(3-thienyl)piperidine-2,4-dione;
3-(2-chlorophenyl)sulfanyl-6-[6-(4-fluoro-3-methyl-phenoxy)-2-pyridyl]-6-(3-thienyl)piperidine-2,4-dione;
3-(2-chlorophenyl)sulfanyl-6-[6-(4-fluoroanilino)-2-pyridyl]-1-methyl-6-(3-thienyl)piperidine-2,4-dione;
3-(2-chlorophenyl)sulfanyl-6-[6-(cyclohexoxy)-2-pyridyl]-6-(3-thienyl)piperidine-2,4-dione;
3-(2-chlorophenyl)sulfanyl-6-[6-(4-fluoroanilino)-2-pyridyl]-6-(3-thienyl)piperidine-2,4-dione;
3-(2-chlorophenyl)sulfanyl-6-[6-(4-fluoroanilino)-2-pyridyl]-6-(4-morpholinophenyl)piperidine-2,4-dione;
3-(2-chlorophenyl)sulfanyl-6-[6-(4-fluoroanilino)-5-morpholino-2-pyridyl]-6-(3-thienyl)piperidine-2,4-dione;
3-(2-chlorophenyl)sulfanyl-6-[6-(4-fluorobenzoyl)-2-pyridyl]-6-(3-thienyl)piperidine-2,4-dione;
3-(2-chlorophenyl)sulfanyl-6-[6-(4-fluoro-N-methyl-anilino)-2-pyridyl]-6-(3-thienyl)piperidine-2,4-dione;
3-(2-chlorophenyl)sulfanyl-6-[6-(4-fluorophenoxy)-2-pyridyl]-1-methyl-6-(3-thienyl)piperidine-2,4-dione;
3-(2-chlorophenyl)sulfanyl-6-[6-(4-fluorophenoxy)-2-pyridyl]-6-(1H-pyrazol-3-yl)piperidine-2,4-dione
3-(2-chlorophenyl)sulfanyl-6-[6-(4-fluorophenoxy)-2-pyridyl]-6-(2-hydroxyphenyl)piperidine-2,4-dione;
3-(2-chlorophenyl)sulfanyl-6-[6-(4-fluorophenoxy)-2-pyridyl]-6-(3-thienyl)piperidine-2,4-dione;
3-(2-chlorophenyl)sulfanyl-6-[6-(4-fluorophenoxy)-2-pyridyl]-6-(4-morpholinophenyl)piperidine-2,4-dione;
3-(2-chlorophenyl)sulfanyl-6-[6-(4-fluorophenoxy)-5-morpholino-2-pyridyl]-6-(3-thienyl)piperidine-2,4-dione;
3-(2-chlorophenyl)sulfanyl-6-[6-(4-fluorophenyl)-2-pyridyl]-6-(3-thienyl)piperidine-2,4-dione;
3-(2-chlorophenyl)sulfanyl-6-[6-(4-fluorophenyl)sulfanyl-2-pyridyl]-6-(3-thienyl)piperidine-2,4-dione;
3-(2-chlorophenyl)sulfanyl-6-[6-(4-hydroxy-4-methyl-pentoxy)-2-pyridyl]-6-(3-thienyl)piperidine-2,4-dione;
3-(2-chlorophenyl)sulfanyl-6-[6-(4-iodophenoxy)-2-pyridyl]-6-(3-thienyl)piperidine-2,4-dione;
3-(2-chlorophenyl)sulfanyl-6-[6-(4-methoxycyclohexoxy)-2-pyridyl]-6-(3-thienyl)piperidine-2,4-dione
3-(2-chlorophenyl)sulfanyl-6-[6-(4-methoxy-N-methyl-anilino)-2-pyridyl]-6-(3-thienyl)piperidine-2,4-dione;
3-(2-chlorophenyl)sulfanyl-6-[6-(4-methoxyphenoxy)-2-pyridyl]-6-(3-thienyl)piperidine-2,4-dione;
3-(2-chlorophenyl)sulfanyl-6-[6-(4-methyl sulfanylphenoxy)-2-pyridyl]-6-(3-thienyl)piperidine-2,4-dione;
3-(2-chlorophenyl)sulfanyl-6-[6-(4-pyridyl)-2-pyridyl]-6-(3-thienyl)piperidine-2,4-dione;
3-(2-chlorophenyl)sulfanyl-6-[6-(4-pyridylmethoxy)-2-pyridyl]-6-(3-thienyl)piperidine-2,4-dione;
3-(2-chlorophenyl)sulfanyl-6-[6-(5-fluorotetralin-1-yl)oxy-2-pyridyl]-6-(3-thienyl)piperidine-2,4-dione
3-(2-chlorophenyl)sulfanyl-6-[6-(5-isoquinolyloxy)-2-pyridyl]-6-(3-thienyl)piperidine-2,4-dione;
3-(2-chlorophenyl)sulfanyl-6-[6-(5-quinolyloxy)-2-pyridyl]-6-(3-thienyl)piperidine-2,4-dione;
3-(2-chlorophenyl)sulfanyl-6-[6-(6-fluorotetralin-1-yl)oxy-2-pyridyl]-6-(3-thienyl)piperidine-2,4-dione
3-(2-chlorophenyl)sulfanyl-6-[6-(6-quinolyloxy)-2-pyridyl]-6-(3-thienyl)piperidine-2,4-dione;
3-(2-chlorophenyl)sulfanyl-6-[6-(7-fluorotetralin-1-yl)oxy-2-pyridyl]-6-(3-thienyl)piperidine-2,4-dione
3-(2-chlorophenyl)sulfanyl-6-[6-(8-fluorochroman-4-yl)oxy-2-pyridyl]-6-(3-thienyl)piperidine-2,4-dione;
3-(2-chlorophenyl)sulfanyl-6-[6-(8-hydroxy-3,4-dihydro-2H-quinolin-1-yl)-2-pyridyl]-6-(3-thienyl)piperidine-2,4-dione;
3-(2-chlorophenyl)sulfanyl-6-[6-(8-isoquinolyloxy)-2-pyridyl]-6-(3-thienyl)piperidine-2,4-dione;
3-(2-chlorophenyl)sulfanyl-6-[6-(8-quinolyloxy)-2-pyridyl]-6-(3-thienyl)piperidine-2,4-dione;
3-(2-chlorophenyl)sulfanyl-6-[6-(cyclobutoxy)-2-pyridyl]-6-(3-thienyl)piperidine-2,4-dione;
3-(2-chlorophenyl)sulfanyl-6-[6-(cyclobutylmethoxy)-2-pyridyl]-6-(3-thienyl)piperidine-2,4-dione;
3-(2-chlorophenyl)sulfanyl-6-[6-(cycloheptoxy)-2-pyridyl]-6-(3-thienyl)piperidine-2,4-dione;
3-(2-chlorophenyl)sulfanyl-6-[6-(cyclohexoxy)-2-pyridyl]-6-(3-thienyl)piperidine-2,4-dione;
3-(2-chlorophenyl)sulfanyl-6-[6-(cyclohexoxy)-2-pyridyl]-6-(4-morpholinophenyl)piperidine-2,4-dione;
3-(2-chlorophenyl)sulfanyl-6-[6-(cyclohexoxy)-2-pyridyl]-6-[4-(1-piperidyl)phenyl]piperidine-2,4-dione;
3-(2-chlorophenyl)sulfanyl-6-[6-(cyclohexylamino)-2-pyridyl]-6-(3-thienyl)piperidine-2,4-dione;
3-(2-chlorophenyl)sulfanyl-6-[6-(cyclohexylmethoxy)-2-pyridyl]-6-(3-thienyl)piperidine-2,4-dione;
3-(2-chlorophenyl)sulfanyl-6-[6-(cyclopentoxy)-2-pyridyl]-6-(3-thienyl)piperidine-2,4-dione;
3-(2-chlorophenyl)sulfanyl-6-[6-(cyclopentylamino)-2-pyridyl]-6-(3-thienyl)piperidine-2,4-dione;
3-(2-chlorophenyl)sulfanyl-6-[6-(cyclopentylmethoxy)-2-pyridyl]-6-(3-thienyl)piperidine-2,4-dione;
3-(2-chlorophenyl)sulfanyl-6-[6-(cyclopropylmethoxy)-2-pyridyl]-6-(3-thienyl)piperidine-2,4-dione;
3-(2-chlorophenyl)sulfanyl-6-[6-(dimethylamino)-2-pyridyl]-6-(3-thienyl)piperidine-2,4-dione;
3-(2-chlorophenyl)sulfanyl-6-[6-(N-ethyl-4-fluoro-anilino)-2-pyridyl]-6-(3-thienyl)piperidine-2,4-dione;
3-(2-chlorophenyl)sulfanyl-6-[6-(oxetan-3-ylmethoxy)-2-pyridyl]-6-(3-thienyl)piperidine-2,4-dione;
3-(2-chlorophenyl)sulfanyl-6-[6-(tetrahydrofuran-2-ylmethoxy)-2-pyridyl]-6-(3-thienyl)piperidine-2,4-dione;
3-(2-chlorophenyl)sulfanyl-6-[6-(tetrahydrofuran-3-yl amino)-2-pyridyl]-6-(3-thienyl)piperidine-2,4-dione;
3-(2-chlorophenyl)sulfanyl-6-[6-(tetrahydropyran-4-ylamino)-2-pyridyl]-6-(3-thienyl)piperidine-2,4-dione;
3-(2-chlorophenyl)sulfanyl-6-[6-(tetrahydropyran-4-ylmethoxy)-2-pyridyl]-6-(3-thienyl)piperidine-2,4-dione;
3-(2-chlorophenyl)sulfanyl-6-[6-(tetrahydropyran-4-ylmethyl)-2-pyridyl]-6-(3-thienyl)piperidine-2,4-dione;
3-(2-chlorophenyl)sulfanyl-6-[6-(thiazol-2-ylamino)-2-pyridyl]-6-(3-thienyl)piperidine-2,4-dione;
3-(2-chlorophenyl)sulfanyl-6-[6-[(1,5-dimethylpyrazol-3-yl)amino]-2-pyridyl]-6-(3-thienyl)piperidine-2,4-dione;
3-(2-chlorophenyl)sulfanyl-6-[6-[(1-methyl-1,2,4-triazol-3-yl)amino]-2-pyridyl]-6-(3-thienyl)piperidine-2,4-dione;
3-(2-chlorophenyl)sulfanyl-6-[6-[(1-methylcyclopropyl)methoxy]-2-pyridyl]-6-(3-thienyl)piperidine-2,4-dione;
3-(2-chlorophenyl)sulfanyl-6-[6-[(1-methylimidazol-2-yl)amino]-2-pyridyl]-6-(3-thienyl)piperidine-2,4-dione;
3-(2-chlorophenyl)sulfanyl-6-[6-[(1-methylimidazol-2-yl)methoxy]-2-pyridyl]-6-(3-thienyl)piperidine-2,4-dione;
3-(2-chlorophenyl)sulfanyl-6-[6-[(1-methylpyrazol-3-yl)amino]-2-pyridyl]-6-(3-thienyl)piperidine-2,4-dione;
3-(2-chlorophenyl)sulfanyl-6-[6-[(2,4-difluorophenyl)methyl]-2-pyridyl]-6-(3-thienyl)piperidine-2,4-di one;
3-(2-chlorophenyl)sulfanyl-6-[6-[(2,5-dimethylpyrazol-3-yl)amino]-2-pyridyl]-6-(3-thienyl)piperidine-2,4-dione;
3-(2-chlorophenyl)sulfanyl-6-[6-[(2-methylcyclopropyl)methoxy]-2-pyridyl]-6-(3-thienyl)piperidine-2,4-dione;
3-(2-chlorophenyl)sulfanyl-6-[6-[(2-methylpyrazol-3-yl)amino]-2-pyridyl]-6-(3-thienyl)piperidine-2,4-dione;
3-(2-chlorophenyl)sulfanyl-6-[6-[(3,3-difluorocyclobutyl)methoxy]-2-pyridyl]-6-(3-thienyl)piperidine-2,4-dione;
3-(2-chlorophenyl)sulfanyl-6-[6-[(3,4-difluorophenyl)methyl]-2-pyridyl]-6-(3-thienyl)piperidine-2,4-dione;
3-(2-chlorophenyl)sulfanyl-6-[6-[(3,5-difluorophenyl)methyl]-2-pyridyl]-6-(3-thienyl)piperidine-2,4-dione;
3-(2-chlorophenyl)sulfanyl-6-[6-[(3-ethyloxetan-3-yl)methoxy]-2-pyridyl]-6-(3-thienyl)piperidine-2,4-dione;
5-(2-chlorophenyl)sulfanyl-4-hydroxy-2-[6-(4-methoxycyclohexoxy)-2-pyridyl]-2-(3-thienyl)-1,3-dihydropyridin-6-one;
3-(2-chlorophenyl)sulfanyl-6-[6-[(3-fluoro-5-methoxy-phenyl)methyl]-2-pyridyl]-6-(3-thienyl)piperidine-2,4-dione;
3-(2-chlorophenyl)sulfanyl-6-[6-[(3-fluorophenyl)methyl]-2-pyridyl]-6-(3-thienyl)piperidine-2,4-dione
3-(2-chlorophenyl)sulfanyl-6-[6-[(4-fluoro-3-methoxy-phenyl)methyl]-2-pyridyl]-6-(3-thienyl)piperidine-2,4-dione;
3-(2-chlorophenyl)sulfanyl-6-[6-[(4-fluorophenyl)methoxy]-2-pyridyl]-6-(3-thienyl)piperidine-2,4-dione;
3-(2-chlorophenyl)sulfanyl-6-[6-[(4-fluorophenyl)methyl]-2-pyridyl]-6-(3-thienyl)piperidine-2,4-dione
3-(2-chlorophenyl)sulfanyl-6-[6-[(4-fluorophenyl)methylamino]-2-pyridyl]-6-(3-thienyl)piperidine-2,4-dione;
3-(2-chlorophenyl)sulfanyl-6-[6-[(4-methylthiazol-2-yl)amino]-2-pyridyl]-6-(3-thienyl)piperidine-2,4-dione;
3-(2-chlorophenyl)sulfanyl-6-[6-[(5-fluoro-3-pyridyl)oxy]-2-pyridyl]-6-(3-thienyl)piperidine-2,4-dione
3-(2-chlorophenyl)sulfanyl-6-[6-[(5-fluoro-8-quinolyl)oxy]-2-pyridyl]-6-(3-thienyl)piperidine-2,4-dione;
3-(2-chlorophenyl)sulfanyl-6-[6-[(5-methyl-1H-imidazol-2-yl)amino]-2-pyridyl]-6-(3-thienyl)piperidine-2,4-dione;
3-(2-chlorophenyl)sulfanyl-6-[6-[(5-methylthiazol-2-yl)amino]-2-pyridyl]-6-(3-thienyl)piperidine-2,4-dione;
3-(2-chlorophenyl)sulfanyl-6-[6-[(5-oxotetrahydrofuran-2-yl)methoxy]-2-pyridyl]-6-(3-thienyl)piperidine-2,4-dione;
3-(2-chlorophenyl)sulfanyl-6-[6-[(6-fluoro-3-pyridyl)amino]-2-pyridyl]-6-(3-thienyl)piperidine-2,4-dione;
3-(2-chlorophenyl)sulfanyl-6-[6-[(6-fluoro-5-methyl-3-pyridyl)amino]-2-pyridyl]-6-(3-thienyl)piperidine-2,4-dione;
3-(2-chlorophenyl)sulfanyl-6-[6-[[3-(hydroxymethyl)phenyl]methyl]-2-pyridyl]-6-(3-thienyl)piperidine-2,4-dione;
3-(2-chlorophenyl)sulfanyl-6-[6-[[4-(hydroxymethy)cycohexyl)cyclohexyl]methoxy]-2-pyridyl]-6-(3-thienyl)piperidine-2,4-dione;
3-(2-chlorophenyl)sulfanyl-6-[6-[1-(3,4-difluorophenyl)ethoxy]-2-pyridyl]-6-(3-thienyl)piperidine-2,4-dione;
3-(2-chlorophenyl)sulfanyl-6-[6-[1-(3-fluorophenyl)ethoxy]-2-pyridyl]-6-(3-thienyl)piperidine-2,4-dione;
3-(2-chlorophenoxy)-6-[6-(4-fluoroanilino)-2-pyridyl]-6-(3-thienyl)piperidine-2,4-dione;
3-(2-chlorophenyl)sulfanyl-6-[6-[1-(4-fluorophenyl)ethoxy]-2-pyridyl]-6-(3-thienyl)piperidine-2,4-dione;
3-(2-chlorophenyl)sulfanyl-6-[6-[1-(4-fluorophenyl)ethylamino]-2-pyridyl]-6-(3-thienyl)piperidine-2,4-dione;
3-(2-chlorophenyl)sulfanyl-6-[6-[1-(4-fluorophenyl)propoxy]-2-pyridyl]-6-(3-thienyl)piperidine-2,4-dione;
3-(2-chlorophenyl)sulfanyl-6-[6-[1-(4-fluorophenyl)propylamino]-2-pyridyl]-6-(3-thienyl)piperidine-2,4-dione;
3-(2-chlorophenyl)sulfanyl-6-[6-[2-(1H-pyrazol-4-yl)phenoxy]-2-pyridyl]-6-(3-thienyl)piperidine-2,4-dione;
3-(2-chlorophenyl)sulfanyl-6-[6-[2-(1-methylcyclopropyl)ethoxy]-2-pyridyl]-6-(3-thienyl)piperidine-2,4-dione;
3-(2-chlorophenyl)sulfanyl-6-[6-[2-(2,2-difluorocyclopropyl)ethoxy]-2-pyridyl]-6-(3-thienyl)piperidine-2,4-dione;
3-(2-chlorophenyl)sulfanyl-6-[6-[2-(2,2-dimethyl-1,3-dioxolan-4-yl)ethoxy]-2-pyridyl]-6-(3-thienyl)piperidine-2,4-dione;
3-(2-chlorophenyl)sulfanyl-6-[6-[2-(2-oxopyrrolidin-1-yl)ethoxy]-2-pyridyl]-6-(3-thienyl)piperidine-2,4-dione;
3-(2-chlorophenyl)sulfanyl-6-[6-[2-(3-methyltriazol-4-yl)phenoxy]-2-pyridyl]-6-(3-thienyl)piperidine-2,4-dione;
3-(2-chlorophenyl)sulfanyl-6-[6-[2-(4-fluorophenyl)ethyl]-2-pyridyl]-6-(3-thienyl)piperidine-2,4-dione
3-(2-chlorophenyl)sulfanyl-6-[6-[2-(cyclopropylmethoxy)-4-fluoro-phenoxy]-2-pyridyl]-6-(3-thienyl)piperidine-2,4-dione;
3-(2-chlorophenyl)sulfanyl-6-[6-[2-(cyclopropylmethyl)-4-fluoro-phenoxy]-2-pyridyl]-6-(3-thienyl)piperidine-2,4-dione;
3-(2-chlorophenyl)sulfanyl-6-[6-[2-(methoxymethyl)phenoxy]-2-pyridyl]-6-(3-thienyl)piperidine-2,4-dione;
3-(2-chlorophenyl)sulfanyl-6-[6-[2-(oxetan-3-yl)ethoxy]-2-pyridyl]-6-(3-thienyl)piperidine-2,4-dione;
3-(2-chlorophenyl)sulfanyl-6-[6-[3-(1-hydroxyethyl)anilino]-2-pyridyl]-6-(3-thienyl)piperidine-2,4-dione;
3-(2-chlorophenyl)sulfanyl-6-[6-[3-(difluoromethyl)-4-fluoro-phenoxy]-2-pyridyl]-6-(3-thienyl)piperidine-2,4-dione;
3-(2-chlorophenyl)sulfanyl-6-[6-[3-(difluoromethyl)phenoxy]-2-pyridyl]-6-(3-thienyl)piperidine-2,4-dione;
3-(2-chlorophenyl)sulfanyl-6-[6-[3-(hydroxymethyl)anilino]-2-pyridyl]-6-(3-thienyl)piperidine-2,4-dione;
3-(2-chlorophenyl)sulfanyl-6-[6-[3-(hydroxymethyl)-N-methyl-anilino]-2-pyridyl]-6-(3-thienyl)piperidine-2,4-dione;
3-(2-chlorophenyl)sulfanyl-6-[6-[3-fluoro-5-(hydroxymethyl)phenoxy]-2-pyridyl]-6-(3-thienyl)piperidine-2,4-dione;
3-(2-chlorophenyl)sulfanyl-6-[6-[4-fluoro-3-(hydroxymethyl)anilino]-2-pyridyl]-6-(3-thienyl)piperidine-2,4-dione;
3-(2-chlorophenyl)sulfanyl-6-[6-[4-fluoro-3-(trifluoromethyl)phenoxy]-2-pyridyl]-6-(3-thienyl)piperidine-2,4-dione;
3-(2-chlorophenyl)sulfanyl-6-[6-[6-(hydroxymethyl)indolin-1-yl]-2-pyridyl]-6-(3-thienyl)piperidine-2,4-dione;
3-(2-chlorophenyl)sulfanyl-6-[6-[N-methyl-3-(trifluoromethyl)anilino]-2-pyridyl]-6-(3-thienyl)piperidine-2,4-dione;
3-(2-chlorophenyl)sulfanyl-6-phenyl-6-(3-thienyl)piperidine-2,4-dione;
3-(2-chlorophenyl)sulfanyl-6-phenyl-6-thiazol-4-yl-piperidine-2,4-dione;
3-(2-chlorophenyl)sulfanyl-6-thiazol-4-yl-6-(3-thienyl)piperidine-2,4-dione;
4-[3-[5-(2-chlorophenyl)sulfanyl-2-(4-morpholinophenyl)-4,6-dioxo-2-piperidyl]phenyl]-N,N-dimethyl-benzenesulfonamide;
4-[3-[5-(2-chlorophenyl)sulfanyl-4,6-dioxo-2-(3-thienyl)-2-piperidyl]phenyl]-N,N-dimethyl-benzenesulfonamide;
4-[6-[5-(2-chlorophenyl)sulfanyl-4,6-dioxo-2-(3-thienyl)-2-piperidyl]-2-pyridyl]-N,N-dimethyl-benzenesulfonamide;
3-(2-chlorophenyl)sulfanyl-6-(3-thienyl)-6-[6-[3-(trifluoromethyl)phenoxy]-2-pyridyl]piperidine-2,4-dione;
6-(3-aminophenyl)-3-(2-chlorophenyl)sulfanyl-6-(3-thienyl)piperidine-2,4-dione;
6-(3-anilinophenyl)-3-(2-chlorophenyl)sulfanyl-6-(3-thienyl)piperidine-2,4-dione;
6-(3-bromo-4-morpholino-phenyl)-3-(2-chlorophenyl)sulfanyl-6-(3-thienyl)piperidine-2,4-dione;
6-(3-bromophenyl)-3-(2-chlorophenyl)sulfanyl-1-methyl-6-(3-thienyl)piperidine-2,4-dione;
6-(3-bromophenyl)-3-(2-chlorophenyl)sulfanyl-6-(3-thienyl)piperidine-2,4-dione;
6-(5-bromo-6-morpholino-3-pyridyl)-3-(2-chlorophenyl)sulfanyl-6-(3-thienyl)piperidine-2,4-dione;
6-(6-benzyl-2-pyridyl)-3-(2-chlorophenyl)sulfanyl-6-(3-thienyl)piperidine-2,4-dione;
6-(6-benzyloxy-2-pyridyl)-3-(2-chlorophenyl)sulfanyl-6-(3-thienyl)piperidine-2,4-dione;
6-(6-bromo-2-pyridyl)-3-(2-chloro-5-hydroxy-phenyl)sulfanyl-6-(3-thienyl)piperidine-2,4-dione;
6-(6-bromo-2-pyridyl)-3-(2-chlorophenyl)sulfanyl-6-(3-thienyl)piperidine-2,4-dione;
6-(6-bromo-5-morpholino-2-pyridyl)-3-(2-chlorophenyl)sulfanyl-6-(3-thienyl)piperidine-2,4-dione;
6-[3-chloro-5-(4-fluoroanilino)phenyl]-3-(2-chlorophenyl)sulfanyl-6-(3-thienyl)piperidine-2,4-dione;
6-[4-(1,3,3a,4,6,6a-hexahydrofuro[3,4-c]pyrrol-5-yl)phenyl]-3-(2-chlorophenyl)sulfanyl-6-(3-thienyl)piperidine-2,4-dione;
6-[4-(2-azaspiro[3.3]heptan-2-yl)phenyl]-3-(2-chlorophenyl)sulfanyl-6-(3-thienyl)piperidine-2,4-dione;
6-[4-(3-azabicyclo[2.1.1]hexan-3-yl)phenyl]-3-(2-chlorophenyl)sulfanyl-6-(3-thienyl)piperidine-2,4-dione;
6-[4-(4-acetylpiperazin-1-yl)phenyl]-3-(2-chlorophenyl)sulfanyl-6-(3-thienyl)piperidine-2,4-dione;
6-[5-(2-chlorophenyl)sulfanyl-4,6-dioxo-2-(3-thienyl)-2-piperidyl]-N-(cyclopropylmethyl)pyridine-2-carboxamide;
6-[6-(2-amino-5-methyl-imidazol-1-yl)-2-pyridyl]-3-(2-chlorophenyl)sulfanyl-6-(3-thienyl)piperidine-2,4-dione;
6-[6-(2-bromophenoxy)-2-pyridyl]-3-(2-chlorophenyl)sulfanyl-6-(3-thienyl)piperidine-2,4-dione;
6-[6-(2-chloro-3,4-difluoro-anilino)-2-pyridyl]-3-(2-chlorophenyl)sulfanyl-6-(3-thienyl)piperidine-2,4-dione;
6-[6-(2-chloro-4-fluoro-anilino)-2-pyridyl]-3-(2-chlorophenyl)sulfanyl-6-(3-thienyl)piperidine-2,4-dione;
6-[6-(2-chloro-4-fluoro-phenoxy)-2-pyridyl]-3-(2-chlorophenyl)sulfanyl-6-(3-thienyl)piperidine-2,4-dione;
6-[6-(2-tert-butoxyethoxy)-2-pyridyl]-3-(2-chlorophenyl)sulfanyl-6-(3-thienyl)piperidine-2,4-dione;
6-[6-(3-bromo-4-fluoro-phenoxy)-2-pyridyl]-3-(2-chlorophenyl)sulfanyl-6-(3-thienyl)piperidine-2,4-di one;
6-[6-(3-chloro-4-fluoro-anilino)-2-pyridyl]-3-(2-chlorophenyl)sulfanyl-6-(3-thienyl)piperidine-2,4-dione;
6-[6-(3-chloro-4-fluoro-phenoxy)-2-pyridyl]-3-(2-chlorophenyl)sulfanyl-6-(3-thienyl)piperidine-2,4-dione;
6-[6-(3-chlorophenoxy)-2-pyridyl]-3-(2-chlorophenyl)sulfanyl-6-(3-thienyl)piperidine-2,4-dione;
6-[6-(4-bromo-2-chloro-phenoxy)-2-pyridyl]-3-(2-chlorophenyl)sulfanyl-6-(3-thienyl)piperidine-2,4-dione;
6-[6-(4-bromo-2-fluoro-phenoxy)-2-pyridyl]-3-(2-chlorophenyl)sulfanyl-6-(3-thienyl)piperidine-2,4-dione;
6-[6-(4-chloro-N-methyl-anilino)-2-pyridyl]-3-(2-chlorophenyl)sulfanyl-6-(3-thienyl)piperidine-2,4-dione;
6-[6-(4-chlorophenoxy)-2-pyridyl]-3-(2-chlorophenyl)sulfanyl-6-(3-thienyl)piperidine-2,4-dione;
6-[6-(7-bromotetralin-1-yl)oxy-2-pyridyl]-3-(2-chlorophenyl)sulfanyl-6-(3-thienyl)piperidine-2,4-dione;
6-[6-[(2-chloro-6-fluoro-3-pyridyl)oxy]-2-pyridyl]-3-(2-chlorophenyl)sulfanyl-6-(3-thienyl)piperidine-2,4-dione;
6-[6-[(4-chloro-3-fluoro-phenyl)methyl]-2-pyridyl]-3-(2-chlorophenyl)sulfanyl-6-(3-thienyl)piperidine-2,4-dione;
6-[6-[[1-(3-chloro-4-fluoro-phenyl)-2-hydroxy-ethyl]amino]-2-pyridyl]-3-(2-chlorophenyl)sulfanyl-6-(3-thienyl)piperidine-2,4-dione;
6-[6-[1-(3-chloro-4-fluoro-phenyl)propylamino]-2-pyridyl]-3-(2-chlorophenyl)sulfanyl-6-(3-thienyl)piperidine-2,4-dione;
6-[6-[1-(4-chlorophenyl)ethoxy]-2-pyridyl]-3-(2-chlorophenyl)sulfanyl-6-(3-thienyl)piperidine-2,4-dione;
N-[6-[5-(2-chlorophenyl)sulfanyl-4,6-dioxo-2-(3-thienyl)-2-piperidyl]-2-pyridyl]azetidine-1-sulfonamide tert-butyl;
5-(2-chlorophenyl)sulfanyl-4-hydroxy-2-[4-(1-piperidyl)phenyl]-2-(3-thienyl)-1,3-dihydropyridin-6-one; and
N-[6-[5-(2-chlorophenyl)sulfanyl-4,6-dioxo-2-(3-thienyl)-2-piperidyl]-2-pyridyl]carbamate.

In an embodiment, the invention relates to a compound according to the invention for use as therapeutically active substance.

In an embodiment, the invention relates to a pharmaceutical composition comprising a compound according to the invention and a therapeutically inert carrier.

In an embodiment, the invention relates to a compound according to the invention for the treatment or prophylaxis of cancer.

In an embodiment, the invention relates to the use of a compound according to the invention for the preparation of a medicament for the treatment or prophylaxis of cancer.

In an embodiment, the invention relates to a compound according to the invention for the treatment or prophylaxis of cancer.

In an embodiment, the invention relates to a method for the treatment or prophylaxis of cancer which method comprises administering an effective amount of a compound according to the invention.

In an embodiment, the invention cancer is selected from the groups consisting of the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, pancreatic, myeloid disorders, lymphoma, hairy cells, buccal cavity, naso-pharyngeal, pharynx, lip, tongue, mouth, small intestine, colon-rectum, large intestine, rectum, brain and central nervous system, Hodgkin's, leukemia, bronchus, thyroid, liver and intrahepatic bile duct, hepatocellular, gastric, glioma/glioblastoma, endometrial, melanoma, kidney and renal pelvis, urinary bladder, uterine corpus, uterine cervix, multiple myeloma, acute myelogenous leukemia (AML), chronic lymphoid leukemia, chronic myelogenous leukemia, lymphocytic leukemia, myeloid leukemia, oral cavity and pharynx, non-Hodgkin lymphoma, melanoma, or villous colon adenoma

### Pharmaceutical Formulations

In order to use a Formula (I) compound for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of Formula (I) having the desired degree of purity may optionally be mixed with pharmaceutically acceptable diluents, carriers, excipients or stabilizers (Remington's Pharmaceutical Sciences (1980) 16^{th} edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound ordinarily can be stored as a solid composition, a lyophilized formulation or as an aqueous solution.

The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the hyperproliferative disorder.

As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations of compounds of Formula (I) may be prepared. Suitable examples of sustained-release preparations include semipermeable matrices of solid hydrophobic polymers containing a compound of Formula (I), which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of Formula (I) suitable for oral administration may be prepared as discrete units such as pills, capsules, cachets or tablets each containing a predetermined amount of a compound of Formula (I). Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formula (I) intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include a polyhydric alcohol, i.e., an alcohol having two or more hydroxy groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs. The oily phase of the emulsions of this invention may be constituted from known ingredients in a known manner, including a mixture of at least one emulsifier with a fat or an oil, or with both a fat and an oil. A hydrophilic emulsifier included together with a lipophilic emulsifier acts as a stabilizer. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of Formula (I) compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formula (I) may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use.

Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The compounds of Formula (I) may be employed alone or in combination with other therapeutic agents for the treatment of a disease or disorder described herein, such as inflammation or a hyperproliferative disorder (e.g., cancer). In certain embodiments, a compound of Formula (I) is combined in a pharmaceutical combination formulation, or dosing regimen as combination therapy, with a second therapeutic compound that has anti-inflammatory or anti-hyperproliferative properties or that is useful for treating an inflammation, immune-response disorder, or hyperproliferative disorder (e.g., cancer). The second therapeutic agent may be an NSAID anti-inflammatory agent. The second therapeutic agent may be a chemotherapeutic agent. The second compound of the pharmaceutical combination formulation or dosing regimen preferably has complementary activities to the compound of Formula (I) such that they do not adversely affect each other. Such compounds are suitably present in combination in amounts that are effective for the purpose intended. In an embodiment, a composition of this invention comprises a compound of Formula (I), or a stereoisomer, tautomer, or pharmaceutically acceptable salt or prodrug thereof, in combination with a therapeutic agent such as an NSAID.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other therapeutic agents or treatments.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes, separate pills or capsules, or separate infusions. In general, during alternation therapy, an effective dosage of each active ingredient is administered sequentially, i.e., serially, whereas in combination therapy, effective dosages of two or more active ingredients are administered together.

In a particular embodiment of therapy, a compound of Formula (I), or a stereoisomer, tautomer, or pharmaceutically acceptable salt or prodrug thereof, may be combined with other therapeutic, hormonal or antibody agents such as those described herein, as well as combined with surgical therapy and radiotherapy. Combination therapies according to the present invention thus comprise the administration of at least one compound of Formula (I), or a stereoisomer, tautomer, or pharmaceutically acceptable salt or prodrug thereof, and the use of at least one other cancer treatment method. The amounts of the compound(s) of Formula (I) and the other pharmaceutically active chemotherapeutic agent(s) and the relative timings of administration will be selected in order to achieve the desired combined therapeutic effect.

### Metabolites of Compounds of Formula (I)

Also falling within the scope of this invention are the in vivo metabolic products of Formula (I) described herein. Such products may result for example from the oxidation, reduction, hydrolysis, amidation, deamidation, esterification, deesterification, enzymatic cleavage, and the like, of the administered compound. Accordingly, the invention includes metabolites of compounds of Formula (I), including compounds produced by a process comprising contacting a compound of this invention with a mammal for a period of time sufficient to yield a metabolic product thereof.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

### Articles of Manufacture

In another embodiment of the invention, an article of manufacture, or “kit”, containing materials useful for the treatment of the diseases and disorders described above is provided. In an embodiment, the kit comprises a container comprising a compound of Formula (I). The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula (I) or a formulation thereof which is effective for treating the condition and may have a sterile access port (for example, the container may be an intravenous solution bag or a vial having a stopper pierceable by a hypodermic injection needle). At least one active agent in the composition is a compound of Formula (I). The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In an embodiment, the label or package inserts indicates that the composition comprising a compound of Formula (I) can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula (I) and, if present, the second pharmaceutical formulation. For example, if the kit comprises a first composition comprising a compound of Formula (I) and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula (I), such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of Formula (I) contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of Formula (I) and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Biological Evaluation

Within the scope of the present invention the inventors have identified LDHA inhibitors.

The relative efficacies of Formula (I) compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC₅₀”. Determination of IC₅₀ values can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a “selective LDHA inhibitor” can be understood to refer to a compound that exhibits a 50% inhibitory concentration (IC₅₀) with respect to LDHA that is at least at least 10-fold lower than the IC₅₀ value with respect to any or all of the other LDHA family members.

Determination of the activity of LDHA kinase activity of Formula (I) compounds is possible by a number of direct and indirect detection methods. The range of IC50 values for inhibition of LDHA was less than 1 nM (nanomolar) to about 10 μM (micromolar). Certain exemplary compounds of the invention had LDHA inhibitory IC₅₀ values less than 10 nM. Certain Formula (I) compounds may have antiproliferative properties and may be useful to treat disorders such as cancer. The Formula (I) compounds may inhibit LDHA in mammals and may be useful for treating human cancer patients.

The Example section of this patent application herein shows Formula (I) compounds that were made, characterized, and tested for inhibition of LDHA and selectivity according to the methods of this invention, and have the corresponding structures and names (ChemBioDraw Ultra, Version 11.0, CambridgeSoft Corp., Cambridge Mass.).

### Preparation of Formula (I) Compounds

The compounds of Formula (I) may be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing Formula (I) compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds of Formula (I) may be prepared singly or as compound libraries comprising at least 2, for example 5 to 1,000 compounds, or 10 to 100 compounds. Libraries of compounds of Formula (I) may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds, or pharmaceutically acceptable salts thereof.

In preparing compounds of Formulas I, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

For illustrative purposes, the following schemes show general methods for preparing compounds of Formula (I) according to the invention, as well as key intermediates. For a more detailed description of the individual reaction steps, see the Examples sections. Those skilled in the art will appreciate that other synthetic routes may be used to synthesize the inventive compounds. Although specific starting materials and reagents are depicted and discussed in the General Procedures, Examples, and schemes, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

### EXAMPLES

The invention will be more fully understood by reference to the following examples. They should not, however, be construed as limiting the scope of the invention.

The chemical reactions described in the Examples may be readily adapted to prepare a number of other LDHA inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting reactive functional groups, by utilizing other suitable reagents known in the art other than those described, and/or by making routine modifications of reaction conditions. Alternatively, other reactions disclosed herein or known in the art will be recognized as having applicability for preparing other compounds of the invention.

¹H NMR spectra were recorded at ambient temperature using an NMR spectrometer, including a Varian Unity Inova (400 MHz) spectrometer with a triple resonance 5 mm probe. Chemical shifts are expressed in ppm relative to tetramethylsilane. The following abbreviations have been used: br=broad signal, s=singlet, d=doublet, dd=double doublet, t=triplet, q=quartet, m=multiplet.

High Pressure Liquid Chromatography/Mass Spectrometry (LCMS) experiments to determine retention times (R_{T}) and associated mass ions may be performed. The spectrometers may have an electrospray source operating in positive and negative ion mode. Additional detection is achieved using an evaporative light scattering detector.

Unless otherwise stated, all reactions were performed under an inert, i.e. argon or nitrogen, atmosphere.

### ABBREVIATIONS

AcOH: Acetic acid; BOC: Di-tert-butyl dicarbonate; DCM: Dichloromethane; DIPEA: Diisopropylethylamine; DMAP: 4-Dimethylaminopyridine; EtOAc: Ethyl acetate; HATU: (2-(7-Aza-1H-benzotriazole-1-yl)-1,1,3,3-tetramethyluronium hexafluorophosphate); HCl: Hydrochloric acid; MeOH: Methanol; NaBH₄: Sodium borohydride, NBS: N-Bromosuccinimide; NH₄Cl: Ammonium chloride; NMR: Nuclear magnetic resonance; Pd(dppf)Cl₂: [1,1′-Bis(diphenylphosphino)ferrocene]dichloropalladium(II), complex with dichloromethane; RT: Room temperature; TFA: Trifluoroacetic acid; THF: Tetrahydrofuran.

### Example 1

### 6-(6-bromo-2-pyridyl)-3-(2-chlorophenyl)sulfanyl-6-(3-thienyl)piperidine-2,4-dione

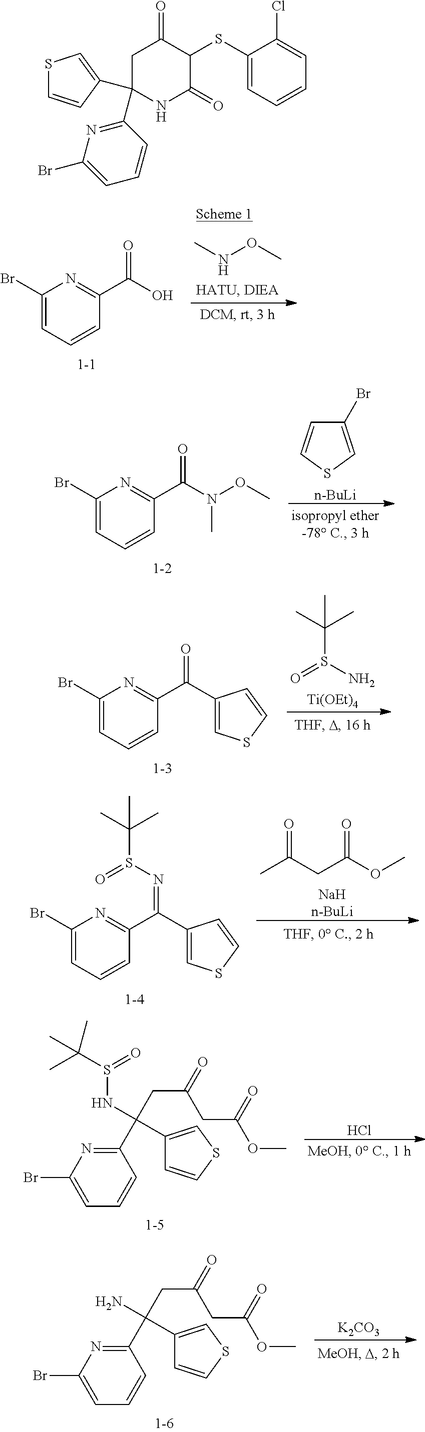

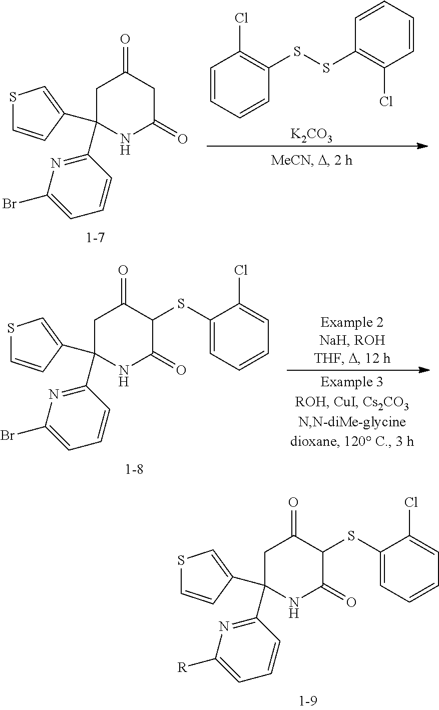

Step A: N,O-Dimethylhydroxylamine hydrochloride (39 g, 0.40 mol), (dimethylamino)-N,N-dimethyl(3H-[1,2,3]triazolo[4,5-b]pyridin-3-yloxy)-methaniminium hexafluorophosphate (152 g, 0.40 mol) and N,N-diisopropylethylamine (130.3 g, 1.01 mol) was added to a solution of 6-bromopicolinic acid (68 g, 0.34 mol) in DCM (1 L). The mixture was stirred at ambient temperature for 3 hours. The reaction mixture was washed with 1 N HCl (600 mL×2), dried over anhydrous Na₂SO₄ and concentrated. The crude residue was purified by silica gel chromatography eluting with a gradient of 10%-30% EtOAc/hexanes to afford 6-bromo-N-methoxy-N-methylpicolinamide (80 g, 0.33 mol, 97% yield) as light color oil.

Step B: n-BuLi (158 mL, 0.4 mol) was slowly added to a solution of 3-bromothiophene (65.2 g, 0.4 mol) in isopropyl ether (1 L) at −78° C. After stirring at −78° C. for 30 min, the reaction mixture was then slowly treated with 6-bromo-N-methoxy-N-methylpicolinamide (80 g, 0.33 mol) and stirred at −78° C. for 3 hours. The reaction mixture was quenched with saturated NH₄Cl (300 mL), then warmed to ambient temperature. The mixture was diluted with EtOAc (400 mL), washed with water (500 mL×2), dried over anhydrous Na₂SO₄ and concentrated. The crude residue was purified by silica gel chromatography eluting with a gradient of 0%-10% EtOAc/hexanes to afford (6-bromopyridin-2-yl)(thiophen-3-yl)methanone (75 g, 0.28 mol, 86% yield) as yellow solid.

Step C: (6-Bromopyridin-2-yl)(thiophen-3-yl)methanone (75 g, 0.28 mol) and Ti(OEt)₄ (191.5 g, 0.84 mol) was added to a solution of 2-methylpropane-2-sulfinamide (67.8 g, 0.56 mol) in THF (1 L). The mixture was heated at 70° C. for 16 hours. The suspension was allowed to cool to ambient temperature. The mixture was pour into ice water, filtered, washed with EtOAc. The filtrate was extracted with EtOAc (500 mL×2), dried over anhydrous Na₂SO₄ and concentrated. The crude was purified by silica gel chromatography eluting with a gradient of 10%-30% EtOAc/hexanes to afford N-((6-bromopyridin-2-yl)(thiophen-3-yl)methylene)-2-methylpropane-2-sulfinamide (80 g, 215.6 mmol, 77% yield) as orange oil.

Step D: Methyl 3-oxobutanoate (50.0 g, 431.2 mmol,) was added to a suspension of NaH (10.35 g, 431.2 mmol,) in THF (1 L) under 0° C. The reaction mixture was then slowly treated with n-BuLi (172 mL, 431.2 mmol,) and stirred under 0° C. for 30 minutes, N-((6-bromopyridin-2-yl)(thiophen-3-yl)methylene)-2-methylpropane-2-sulfinamide (80 g, 215.6 mmol,) was added to the mixture and stirred at 0° C. for another 2 hours. The reaction mixture was quenched with saturated NH₄Cl (500 mL), then warmed to ambient temperature. The mixture was diluted with EtOAc (400 mL), washed with water (500 mL×2), dried over anhydrous Na₂SO₄ and concentrated to afford methyl 5-(6-bromopyridin-2-yl)-5-(1,1-dimethylethylsulfinamido)-3-oxo-5-(thiophen-3-yl)pentanoate (95 g, 194.9 mol, 90% yield) as yellow oil.

Step E: HCl/MeOH (150 mL) was slowly added to a solution of 5-(6-bromopyridin-2-yl)-5-(1,1-dimethylethylsulfinamido)-3-oxo-5-(thiophen-3-yl)pentanoate (95 g, 194.9 mol) in MeOH (1 L) at 0° C. The mixture was stirred at ambient temperature for 1 hour, and then slowly acidified to pH 7 using 2 N NaOH at 0° C. The solvent was removed under vacuum. The crude product was extracted with EtOAc (800 mL×2), dried over anhydrous Na₂SO₄ and concentrated to afford methyl 5-amino-5-(6-bromopyridin-2-yl)-3-oxo-5-(thiophen-3-yl)pentanoate (62 g, 161.9 mmol, 83% yield) as dark color oil.

Step F: Potassium carbonate (67.1 g, 485.7 mmol) was added to a solution of methyl 5-amino-5-(6-bromopyridin-2-yl)-3-oxo-5-(thiophen-3-yl)pentanoate (62 g, 161.9 mmol) in MeOH (800 mL). The mixture was heated at 80° C. for 2 hours. The suspension was allowed to cool to ambient temperature. The solvent was removed under vacuum, the crude product was dissolved in water (1 L), washed with EtOAc (1 L×2). The aqueous layer was acidified to pH 4 using 3 N HCl. The mixture was extracted with EtOAc (800 mL×2). The organic layer was dried over anhydrous Na₂SO₄ and concentrated to afford 6′-bromo-4-hydroxy-2-(thiophen-3-yl)-2,3-dihydro-[2,2′-bipyridin]-6(1H)-one (31 g, 88.3 mmol, 55% yield) as yellow solid.

Step G: Potassium carbonate (36.6 g, 264.9 mmol) and 1,2-bis(2-chlorophenyl)disulfane (15.2 g, 53.0 mmol) was added to a solution of 6′-bromo-4-hydroxy-2-(thiophen-3-yl)-2,3-dihydro-[2,2′-bipyridin]-6(1H)-one (31 g, 88.3 mmol) in MeOH (800 mL). The mixture was heated at 80° C. for 2 hours. The suspension was allowed to cool to ambient temperature. The solvent was removed under vacuum, the crude product was dissolved in water (800 mL), washed with EtOAc (800 mL×2). The aqueous layer was acidified to pH 4 using 3 N HCl. The mixture was extracted with EtOAc (800 mL×2). The organic layer was dried over anhydrous Na₂SO₄ and concentrated to afford 6′-bromo-4-hydroxy-2-(thiophen-3-yl)-2,3-dihydro-[2,2′-bipyridin]-6(1H)-one (38 g, 76.9 mmol, 87% yield) as light color solid.

### Example 2

### 3-(2-chlorophenyl)sulfanyl-6-(6-isopropoxy-2-pyridyl)-6-(3-thienyl)piperidine-2,4-dione

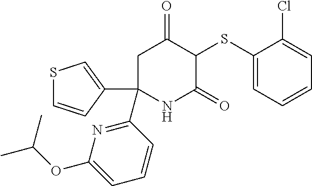 [SMILES: CC(C)Oc1cccc(C2(c3ccsc3)CC(=O)C(Sc3ccccc3Cl)C(=O)N2)n1]

Step A: NaH (73 mg, 3.04 mmol) was added to a solution of propan-2-ol (182 mg, 3.04 mmol) in THF (10 mL) at 0° C. After stirring 30 minutes, 6′-bromo-4-hydroxy-2-(thiophen-3-yl)-2,3-dihydro-[2,2′-bipyridin]-6(1H)-one (300 mg, 0.61 mmol) was added to the mixture at 0° C., and then the mixture was refluxed for 12 hours. The suspension was cooled to 0° C., quenched with water (10 mL), diluted with EtOAc (20 mL), acidified to pH 7 using 1 N HCl, washed with brine, dried over anhydrous Na₂SO₄ and concentrated. The crude residue was purified by preparative HPLC (formic acid) to afford 3-((2-chlorophenyl)thio)-6-(6-isopropoxy-pyridin-2-yl)-6-(thiophen-3-yl)piperidine-2,4-dione (112 mg, 0.24 mmol, 39% yield) as white solid. Mixture of diastereoisomers: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.88 (d, J=16.4 Hz, 1H), 3.45 (d, J=16.4 Hz, 1H), 1.31 (d, J=6.4 Hz, 3H), 1.26 (d, J=6.4 Hz, 3H). LCMS M+1=472.8. Stereoisomer 1: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.89 (d, J=16.4 Hz, 1H), 3.45 (d, J=16.4 HZ, 1H), 1.31 (d, J=6.4 Hz, 3H), 1.26 (d, J=6.4 Hz, 3H). LCMS M+1=472.8. Stereoisomer 2: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.89 (d, J=16.4 Hz, 1H), 3.45 (d, J=16.4 Hz, 1H), 1.31 (d, J=6.4 Hz, 3H), 1.26 (d, J=6.4 Hz, 3H). LCMS M+1=472.9.

### Example 3

### 6-[6-(2-chloro-4-fluoro-phenoxy)-2-pyridyl]-3-(2-chlorophenyl)sulfanyl-6-(3-thienyl)piperidine-2,4-dione

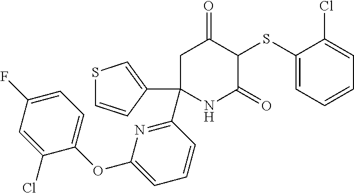 [SMILES: O=C1CC(c2ccsc2)(c2cccc(Oc3ccc(F)cc3Cl)n2)NC(=O)C1Sc1ccccc1Cl]

Step A: 6′-Bromo-5-(2-chloro-phenylsulfanyl)-4-hydroxy-2-thiophen-3-yl-2,3-dihydro-1H-[2,2′]bipyridinyl-6-one (500 mg, 1 mmol), 2-chloro-4-fluoro-phenol (178 mg, 1.2 mmol), 2-(dimethylamino)acetic acid hydrochloride (28 mg, 0.2 mmol), CuI (39 mg, 0.2 mmol) and Cs₂CO₃ (0.99 g, 3 mmol) were combined. Dioxane (5 ml) was added, the mixture was stirred at 120° C. for 3 h under nitrogen atmosphere. After the suspension was cooled to ambient temperature, EtOAc (20 mL) was added, and the mixture was filtered over Celite. The resulting solution was washed three times with brine, dried anhydrous Na₂SO₄, filtered, and the solvent evaporated under reduced pressure. The crude residue was purified by preparative HPLC (formic acid) to give the product (mixture of diastereoisomers, 230 mg, 41%, 10 mg was delivered) as white solid. The mixture of diastereoisomers (220 mg) was purified by SFC (neutral) to give the isomers (stereoisomer 1, 80 mg and stereoisomer 2, 128 mg) as white solid. Mixture of diastereoisomers: ¹H NMR (400 MHz, (CD₃)₂SO) δ 7.93 (dd, J=8.0, 8.0 Hz, 1H), 7.60-7.53 (m, 1H), 7.44 (dd, J=4.8, 2.1 Hz, 1H), 7.37 (d, J=7.6 Hz, 1H), 7.30-7.23 (m, 3H), 7.18 (dd, J=2.8, 1.2 Hz, 1H), 7.04 (d, J=8.4 Hz, 1H), 6.98-6.93 (m, 1H), 6.91 (dd, J=4.2, 1.2 Hz, 1H), 6.78-6.74 (m, 1H), 5.88 (dd, J=7.6, 1.2 Hz, 1H), 3.37 (d, J=16.4 Hz, 1H), 3.13 (d, J=16.4 Hz, 1H). LCMS M+1=558.7. Stereoisomer 1: ¹H NMR (400 MHz, CD₃OD) δ 7.88 (dd, J=8.0, 8.0 Hz, 1H), 7.36 (dd, J=3.9, 2.4 Hz, 1H), 7.35-7.32 (m, 2H), 7.22 (dd, J=8.0, 1.2 Hz, 1H), 7.19-7.09 (m, 3H), 7.07 (d, J=8.2 Hz, 1H), 6.96 (dd, J=3.9, 0.9 Hz, 1H), 6.94 (dd, J=8.0, 1.2 Hz, 1H), 6.81-6.74 (m, 1H), 5.88 (dd, J=8.4, 1.2 Hz, 1H), 3.48 (d, J=16.4 Hz, 1H), 3.20 (d, J=16.4 Hz, 1H). LCMS M+1=558.7. Stereoisomer 2: ¹H NMR (400 MHz, CD₃OD) δ 7.89 (dd, J=8.0, 8.0 Hz, 1H), 7.36 (dd, J=3.9, 2.4 Hz, 1H), 7.35-7.32 (m, 2H), 7.22 (dd, J=8.0, 1.2 Hz, 1H), 7.19-7.09 (m, 3H), 7.07 (d, J=8.2 Hz, 1H), 6.96 (dd, J=3.9, 0.9 Hz, 1H), 6.94 (dd, J=8.0, 1.2 Hz, 1H), 6.81-6.74 (m, 1H), 5.97 (dd, J=8.4, 1.2 Hz, 1H), 3.48 (d, J=16.4 Hz, 1H), 3.20 (d, J=16.4 Hz, 1H). LCMS M+1=558.8.

### Example 4

### 3-(2-chlorophenyl)sulfanyl-6-[6-(cyclohexylamino)-2-pyridyl]-6-(3-thienyl)piperidine-2,4-dione

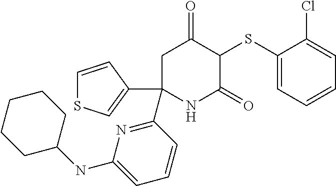 [SMILES: O=C1CC(c2ccsc2)(c2cccc(NC3CCCCC3)n2)NC(=O)C1Sc1ccccc1Cl]

Step A: 6-(6-Bromopyridin-2-yl)-3-((2-chlorophenyl)thio)-6-(thiophen-3-yl)-piperidine-2,4-dione (300 mg, 607.5 μmol), cyclohexanamine (90.4 mg, 911.3 μmol), Brettphos (65.2 mg, 121.5 μmol), Pd₂(dba)₃ (55.6 mg, 60.8 μmol) and NaOtBu (116.8 mg, 1.2 mmol) were combined, dioxane (5 ml) was added. The mixture was stirred at 120° C. for 8 hours under nitrogen atmosphere. After the suspension was cooled to room temperature, ethyl acetate (15 mL) was added, and the mixture was filtered over Celite. The resulting solution was washed three times with brine, dried over sodium sulphate, filtered, and the solvent evaporated under reduced pressure. The residue was purified by preparative HPLC (formic acid) to give the desired product (mixture of diastereoisomers, 75.4 mg, 24%, 7.4 mg was delivered) as yellow solid. The mixture of diastereoisomers (68.0 mg) was purified by SFC (neutral) to give the desired product (stereoisomer 1, 10 mg and stereoisomer 2, 5.8 mg) as yellow solid. Mixture of diastereoisomers: ¹H NMR (400 MHz, CD₃OD) δ 7.88 (dd, J=7.6, 7.6 Hz, 1H), 7.69 (d, J=2.8 Hz, 1H), 7.63 (d, J=2.8 Hz, 1H), 7.28-7.26 (m, 2H), 7.19 (d, J=7.6 Hz, 1H), 7.10 (d, J=7.4 Hz, 1H), 6.85 (d, J=7.6 Hz, 1H), 6.47 (d, J=7.2 Hz, 1H), 6.03 (d, J=8.0 Hz, 1H), 3.70-3.66 (m, 1H), 3.88-3.67 (m, 5H), 3.59 (d, J=16.0 Hz, 2H), 1.93-1.91 (m, 2H), 1.73-1.70 (m, 2H), 1.60-1.41 (m, 2H), 1.28-1.22 (m, 4H). LCMS M+1=511.9. Stereoisomer 1: ¹H NMR (400 MHz, CD₃OD) δ 7.40 (dd, J=7.6, 7.6 Hz, 1H), 7.28 (d, J=2.8 Hz, 1H), 7.18 (d, J=2.8 Hz, 1H), 7.15 (d, J=2.8 Hz, 1H), 6.91 (d, J=7.6 Hz, 1H), 6.77 (d, J=7.4 Hz, 1H), 6.64 (d, J=7.6 Hz, 1H), 6.40 (d, J=7.2 Hz, 1H), 6.06 (d, J=8.0 Hz, 1H), 3.81-3.77 (m, 1H), 3.76 (d, J=16.0 Hz, 1H), 3.41 (d, J=16.0 Hz, 1H), 2.01-1.95 (m, 2H), 1.77-1.73 (m, 2H), 1.70-1.40 (m, 2H), 1.27-1.17 (m, 4H). LCMS M+1=511.8. Stereoisomer 2: ¹H NMR (400 MHz, CD₃OD) δ 7.40 (dd, J=7.6, 7.6 Hz, 1H), 7.28 (d, J=2.8 Hz, 1H), 7.18 (d, J=2.8 Hz, 1H), 7.15 (d, J=2.8 Hz, 1H), 6.91 (d, J=7.6 Hz, 1H), 6.77 (d, J=7.4 Hz, 1H), 6.62 (d, J=7.6 Hz, 1H), 6.40 (d, J=7.2 Hz, 1H), 6.06 (d, J=8.0 Hz, 1H), 3.80-3.78 (m, 1H), 3.76 (d, J=16.0 Hz, 1H), 3.42 (d, J=16.0 Hz, 1H), 2.01-1.95 (m, 2H), 1.74-1.71 (m, 2H), 1.62-1.40 (m, 2H), 1.27-1.17 (m, 4H). LCMS M+1=511.9.

### Example 5

### 3-(2-chlorophenyl)sulfanyl-6-[6-[(3-fluorophenyl)methyl]-2-pyridyl]-6-(3-thienyl)piperidine-2,4-dione

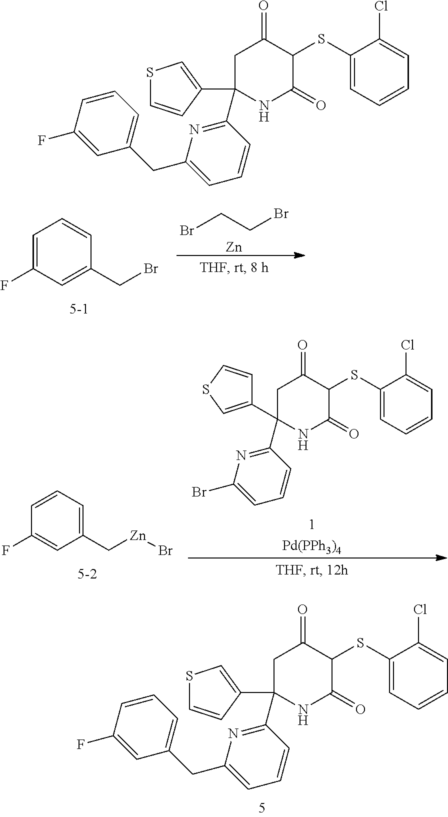

Step A: 1,2-Dibromoethane (100 mg, 0.53 mmol) and 1-(bromomethyl)-3-fluorobenzene (1 g, 5.3 mmol) was added to a suspension of zinc powder (345 mg, 5.3 mmol) in anhydrous THF (10 mL). The reaction mixture was stirred at room temperature for 8 hours. The resultant solution was used directly in the next step.

Step B: (3-Fluorobenzyl)zinc(II) bromide (5.7 mL, 3.04 mmol) was added to a solution of Pd(PPh₃)₄ (69 mg, 0.06 mmol) and 6′-bromo-5-((2-chlorophenyl)thio)-4-hydroxy-2-(thiophen-3-yl)-2,3-dihydro-[2,2′-bipyridin]-6(1H)-one (300 mg, 0.61 mmol) in anhydrous THF (5 mL). The suspension was stirred at room temperature for 12 hours, and then quenched with water, filtered over Celite. The resulting solution was dried over anhydrous Na₂SO₄ and concentrated. The crude residue was purified by preparative HPLC (formic acid) to afford 3-(2-chlorophenyl)sulfanyl-6-[6-[(3-fluorophenyl)methyl]-2-pyridyl]-6-(3-thienyl)piperidine-2,4 dione, (61 mg, 0.12 mmol, 20% yield) as white solid. The mixture of diastereoisomers was purified by SFC (neutral) to give the separated stereoisomers. Mixture of diastereoisomers: ¹H NMR (400 MHz, CD₃OD) δ 7.75 (dd, J=8.0, 8.0 Hz, 1H), 7.41-7.39 (m, 2H), 7.22-7.17 (m, 4H), 7.10-7.09 (m, 2H), 7.08 (d, J=8.0 Hz, 1H), 6.87-6.86 (m, 2H), 6.55 (dd, J=8.0, 0.8 Hz, 1H), 5.82 (dd, J=8.0, 1.6 Hz, 1H), 4.17 (s, 2H), 3.97 (d, J=16.8 Hz, 1H), 3.47 (d, J=16.4 Hz, 1H). LCMS M+1=522.9. Stereoisomer 1: ¹H NMR (400 MHz, CD₃OD) δ 7.74 (dd, J=8.0, 8.0 Hz, 1H), 7.43-7.41 (m, 2H), 7.24-7.17 (m, 4H), 7.15-7.09 (m, 2H), 7.07 (d, J=8.0 Hz, 1H), 6.84-6.82 (m, 2H), 6.55 (dd, J=8.0, 0.8 Hz, 1H), 5.87 (d, J=8.4 Hz, 1H), 4.16 (s, 2H), 3.85 (d, J=16.0 Hz, 1H), 3.45 (d, J=16.4 Hz, 1H). LCMS M+1=522.9. Stereoisomer 2: ¹H NMR (400 MHz, CD₃OD) δ 7.73 (dd, J=8.0, 8.0 Hz, 1H), 7.42-7.36 (m, 2H), 7.24-7.21 (m, 4H), 7.09-7.08 (m, 2H), 7.07 (d, J=8.0 Hz, 1H), 6.84-6.82 (m, 2H), 6.57 (dd, J=8.0, 0.8 Hz, 1H), 5.90 (dd, J=8.0, 1.6 Hz, 1H), 4.16 (s, 2H), 3.78 (d, J=16.0 Hz, 1H), 3.44 (d, J=16.4 Hz, 1H). LCMS M+1=522.9.

### Example 6

### 3-(2-chlorophenyl)sulfanyl-6-[6-(4-fluorobenzoyl)-2-pyridyl]-6-(3-thienyl)piperidine-2,4-dione

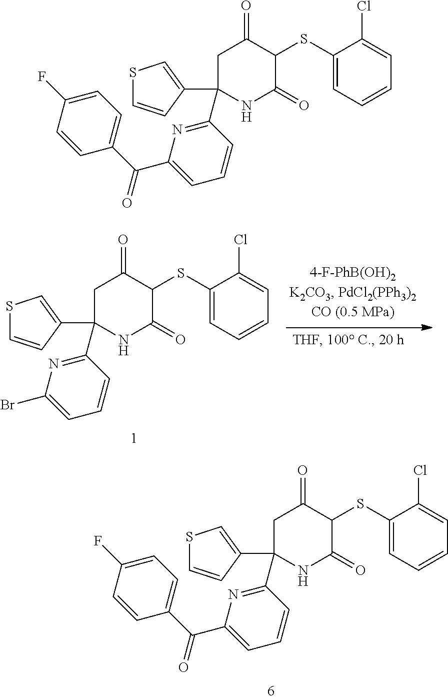

Step A: 6′-Bromo-5-((2-chlorophenyl)thio)-4-hydroxy-2-(thiophen-3-yl)-2,3-dihydro-[2,2′-bipyridin]-6(1H)-one (300 mg, 0.61 mmol) and (4-fluorophenyl)boronic acid (94 mg, 0.67 mmol) was added to a solution of K₂CO₃ (253 mg, 0.83 mmol) and PdCl₂(PPh₃)₂ (21 mg, 0.02 mmol) in THF (6 mL). The mixture was heated at 100° C. for 20 hours under carbon monoxide atmosphere (0.5 MPa). After cooling to room temperature, the reaction was filtered over Celite. The resulting solution was dried over anhydrous Na₂SO₄ and concentrated. The crude residue was purified by preparative HPLC (formic acid) to afford -(2-chlorophenyl)sulfanyl-6-[6-(4-fluorobenzoyl)-2-pyridyl]-6-(3-thienyl)piperidine-2,4-dione (78 mg, 0.15 mmol, 24% yield) as white solid. The mixture of diastereoisomers was purified by SFC (neutral) to give the separated stereoisomers. Mixture of diastereoisomers: mixture of diastereoisomers: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.88 (d, J=16.4 Hz, 1H), 3.45 (d, J=16.4 Hz, 1H), 1.31 (d, J=6.4 Hz, 3H), 1.26 (d, J=6.4 Hz, 3H). LCMS M+1=472.8. Stereoisomer 1: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.89 (d, J=16.4 Hz, 1H), 3.45 (d, J=16.4 HZ, 1H), 1.31 (d, J=6.4 Hz, 3H), 1.26 (d, J=6.4 Hz, 3H). Stereoisomer 2: ¹H NMR (400 MHz, CD₃OD) δ 7.68 (dd, J=8.4, 3.6 Hz, 1H), 7.43 (dd, J=5.2, 2.8 Hz, 1H), 7.26-7.11 (m, 4H), 6.92 (dd, J=8.0, 8.0 Hz, 1H), 6.76-6.67 (m, 2H), 5.97 (dd, J=8.0, 1.2 Hz, 1H), 5.39-5.32 (m, 1H), 3.89 (d, J=16.4 Hz, 1H), 3.45 (d, J=16.4 Hz, 1H), 1.31 (d, J=6.4 Hz, 3H), 1.26 (d, J=6.4 Hz, 3H).

### Example 7

### 3-(2-chlorophenyl)sulfanyl-6-[4-(2-methylmorpholin-4-yl)phenyl]-6-(3-thienyl)piperidine-2,4-dione

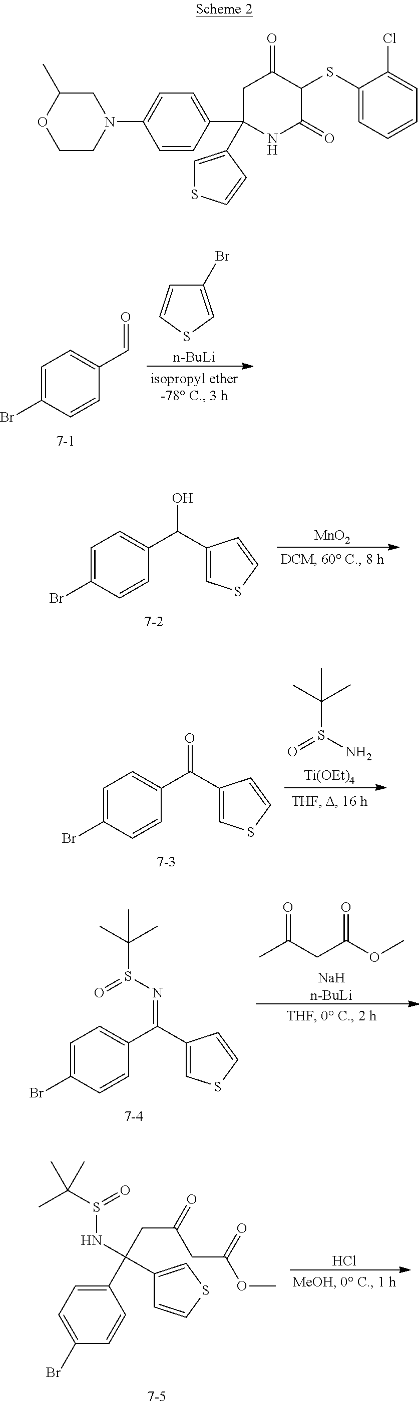

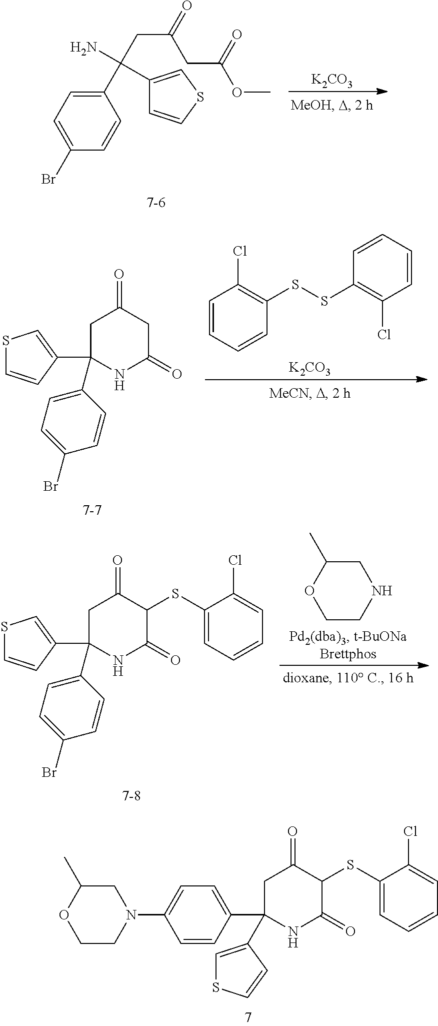

Step A: To a solution of 3-bromothiophene (14.43 g, 220.74 mmol) in anhydrous isopropyl ether (500 mL) was added n-BuLi (88.2 ml, 220.74 mmol) at −78° C. under nitrogen atmosphere. The reaction mixture was stirred for 1 hour. 4-Bromobenzaldehyde (100 g, 183.95 mmol) was added and the reaction mixture was stirred at −78° C. for 2 hours. The reaction was quenched with MeOH and acidified to pH 4 with 1 N HCl, extracted with DCM (100 mL×2). The combined organic layers were dried over anhydrous Na₂SO₄, and concentrated. The crude residue was purified by silica gel chromatography (petroleum ether:EtOAc=3:1) to give (4-bromophenyl)(thiophen-3-yl)methanol (100 g, 69%) as a yellow solid.

Step B: To a solution of (4-bromophenyl)(thiophen-3-yl)methanol (100 g, 371.5 mmol) in CHCl₃ (200 ml) was added MnO₂ (322.9 g, 3715 mmol). The reaction mixture was stirred at 60° C. for 12 hours. After cooling to room temperature, the reaction mixture was filtered over Celite and the filtrate was concentrated under vacuum. The crude residue (86 g, 86% yield) was used in the next step without further purification.

Step C: (E)-N-((4-Bromophenyl)(thiophen-3-yl)methylene)-2-methylpropane-2-sulfinamide was prepared in 86% yield according to the Example 1, Step C substituting (6-bromopyridin-2-yl)(thiophen-3-yl)methanone for (4-bromophenyl)(thiophen-3-yl) methanone.

Step D: Methyl 5-(4-bromophenyl)-5-(1,1-dimethylethylsulfinamido)-3-oxo-5-(thiophen-3-yl) pentanoate was prepared in 85% yield according to the Example 1, Step D: Substituting (Z)—N-((6-bromopyridin-2-yl)(thiophen-3-yl)methylene)-2-methyl-propane-2-sulfinamide for (E)-N-((4-bromophenyl)(thiophen-3-yl)methylene)-2-methylpropane-2-sulfinamide.

Step E:

Methyl 5-amino-5-(4-bromophenyl)-3-oxo-5-(thiophen-3-yl)pentanoate was prepared in 90% yield according to the Example 1, Step E substituting methyl 5-amino-5-(6-bromopyridin-2-yl)-3-oxo-5-(thiophen-3-yl)pentanoate for methyl 5-(4-bromophenyl)-5-(1,1-dimethylethylsulfinamido)-3-oxo-5-(thiophen-3-yl) pentanoate.

Step F:

6-(4-Bromophenyl)-4-hydroxy-6-(thiophen-3-yl)-5,6-dihydropyridin-2(1H)-one was prepared in 75% yield according to the Example 1, Step F substituting 6′-bromo-4-hydroxy-2-(thiophen-3-yl)-2,3-dihydro-[2,2′-bipyridin]-6(1H)-one for methyl 5-amino-5-(4-bromophenyl)-3-oxo-5-(thiophen-3-yl)pentanoate.

Step G:

6-(4-Bromophenyl)-3-((2-chlorophenyl)thio)-4-hydroxy-6-(thiophen-3-yl)-5,6-dihydropyridin-2(1H)-one was prepared in 90% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for 6-(4-bromophenyl)-4-hydroxy-6-(thiophen-3-yl)-5,6-dihydropyridin-2(1H)-one.

Step H: To a solution of 6-(4-bromophenyl)-3-((2-chlorophenyl)thio)-4-hydroxy-6-(thiophen-3-yl)-5,6-dihydropyridin-2(1H)-one (0.25 g, 0.5 mmol) in dioxane (6 mL) was added 2-methylmorpholine (500 mg, 5 mmol), Brettphos (25 mg, 0.05 mmol), Pd₂(dba)₃ (45 mg, 0.05 mmol) and t-BuONa (0.5 g, 5 mmol). The reaction mixture was stirred at 110° C. for 16 hours under nitrogen atmosphere. After cooling to room temperature, the reaction mixture was filtered through a short pad of silica gel. The filtrate was concentrated under vacuum. The crude residue was purified by preparative HPLC (formic acid) to afford the product (10 mg, 3.8% yield) as white solid. ¹H NMR (400 MHz, (CD₃)₂SO) δ 8.35 (s, 1H), 7.57 (d, J=5.2 Hz, 1H), 7.32 (m, 4H), 7.16 (m, 1H), 6.98 (m, 3H), 6.73 (m, 1H), 5.92 (m, 1H), 3.93 (m, 1H), 3.64 (m, 3H), 3.58 (m, 1H), 3.37 (m, 2H), 2.69 (m, 1H), 2.34 (m, 1H), 1.15 (d, J=6.4 Hz, 3H). LCMS M+1=512.9.

### Example 8

### 3-(2-chlorophenyl)sulfanyl-6-[4-(cyclohexen-1-yl)phenyl]-6-(3-thienyl)piperidine-2,4-dione

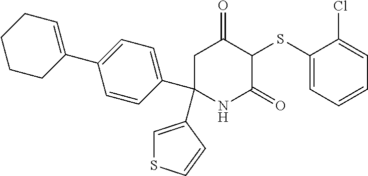 [SMILES: O=C1CC(c2ccc(C3=CCCCC3)cc2)(c2ccsc2)NC(=O)C1Sc1ccccc1Cl]

Step A: To a solution of 6-(4-bromophenyl)-3-((2-chlorophenyl)thio)-6-(thiophen-3-yl) piperidine-2,4-dione (0.25 g, 0.5 mmol) in dioxane (6 mL) and water (2 mL) was added cyclohex-1-en-1-ylboronic acid (126 mg, 1 mmol), Pd(dppf)Cl_{z}(36 mg, 0.05 mmol) and K₂CO₃ (0.27 g, 2 mmol). The reaction mixture was microwaved at 100° C. for 1 hour under nitrogen atmosphere. After cooling to room temperature, the reaction mixture was filtered through a short pad of silica gel. The filtrate was concentrated under vacuum and the crude residue was purified by preparative HPLC (formic acid) to afford the product (11.7 mg, 5% yield). ¹H NMR (400 MHz, (CD₃)₂SO) δ 8.47 (s, 1H), 7.56-7.55 (m, 1H), 7.54-7.39 (m, 2H), 7.32-7.20 (m, 3H), 7.27 (d, J=8 Hz, 1H), 7.14 (dd, J=5.2, 4.8 Hz, 1H), 6.93 (dd, J=7.6, 4.8 Hz, 1H), 6.15 (s, 1H), 5.85 (d, J=8.0 Hz, 1H), 3.39 (s, 2H), 2.47 (s, 2H), 2.33 (s, 2H), 1.71-1.68 (m, 2H), 1.58-1.56 (m, 2H). LCMS M+1=493.9; 495.9.

### Example 9

### 3-(2-chlorophenyl)sulfanyl-6-(4-cyclohexylphenyl)-6-(3-thienyl)piperidine-2,4-dione

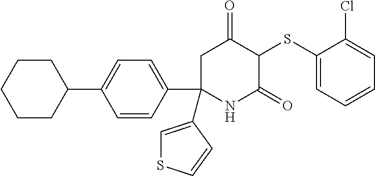 [SMILES: O=C1CC(c2ccc(C3CCCCC3)cc2)(c2ccsc2)NC(=O)C1Sc1ccccc1Cl]

Step A: To a solution of GNT_C349_986 (0.8 g, 1.6 mmol) in acetic acid (20 mL) was added Pd/C (0.1 g). The reaction mixture was stirred at room temperature for 24 hours under hydrogen atmosphere (60 Psi). After relieving the pressure, the reaction mixture was filtrated over Celite and the filtrate was concentrated under vacuum. The crude residue was purified by preparative HPLC (formic acid) to afford the product (10 mg, 1.2% yield) as white solid. ¹H NMR (400 MHz, (CD₃)₂SO) δ 7.49 (s, 1H), 7.35-7.32 (m, 2H), 7.26-7.25 (m, 4H), 7.19 (d, J=8.0 Hz, 1H), 6.93 (dd, J=6.8, 6.8 Hz, 1H), 6.72 (dd, J=6.8, 6.8 Hz, 1H), 5.98 (d, J=6.8 Hz, 1H), 3.45 (s, 2H), 1.96-1.74 (m, 5H), 1.48-1.27 (m, 5H). LCMS M+1=495.8.

### Example 10

### 3-(2-chlorophenyl)sulfanyl-6-[6-[2-(1-methylcyclopropyl)ethoxy]-2-pyridyl]-6-(3-thienyl)piperidine-2,4-dione

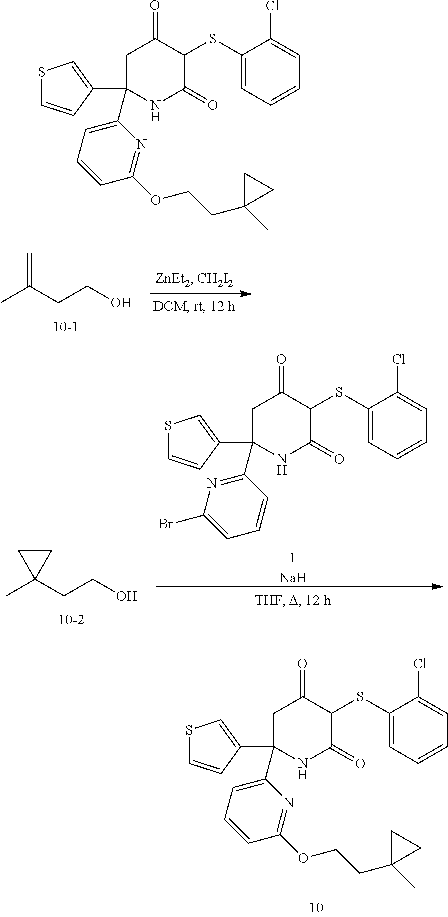

Step A: Diethylzinc (40.6 ml, 40.6 mmol) and diiodomethane (9.3 g, 34.8 mmol) was added to a solution of 3-methylbut-3-en-1-ol (1 g, 11.6 mmol) in DCM (80 mL) at −10° C. The reaction mixture was stirred at 0° C. for 1 hour and then room temperature for additional 12 hours. The reaction was quenched with saturated NH₄Cl, extracted with DCM (50 mL×2), dried over anhydrous Na₂SO₄ and concentrated to afford 2-(1-methylcyclopropyl)ethanol (600 mg, 6 mmol, 52% yield) as light color oil.

Step B: 5-((2-Chlorophenyl)thio)-4-hydroxy-6′-(2-(1-methylcyclopropyl)ethoxy)-2-(thiophen-3-yl)-2,3-dihydro-[2,2′-bipyridin]-6(1H)-one was prepared in 39% yield according to the Example 2, Step A substituting propan-2-ol for 2-(1-methylcyclopropyl)ethanol.

Mixture of diastereoisomers: ¹H NMR (400 MHz, CD₃OD) δ 7.74 (dd, J=8.0, 8.0 Hz, 1H), 7.47 (dd, J=5.2, 3.2 Hz, 1H), 7.30-7.15 (m, 4H), 6.96 (dd, J=8.0, 8.0 Hz, 1H), 6.77-6.75 (m, 2H), 6.01 (dd, J=8.4, 1.6 Hz, 1H), 4.49 (t, J=7.2 Hz, 2H), 3.93 (d, J=16.0 Hz, 1H), 3.48 (d, J=16.4 Hz, 1H), 1.70 (t, J=6.8 Hz, 2H), 1.09 (s, 3H), 0.34-0.23 (m, 4H). Stereoisomer 1: ¹H NMR (400 MHz, CD₃OD) δ 7.50 (dd, J=8.0, 8.0 Hz, 1H), 7.48 (dd, J=5.2, 3.2 Hz, 1H), 7.30-7.22 (m, 2H), 7.06-7.00 (m, 2H), 6.93 (dd, J=8.0, 8.0 Hz, 1H), 6.54-6.52 (m, 2H), 5.79 (dd, J=8.0, 1.6 Hz, 1H), 4.26 (t, J=6.8 Hz, 2H), 3.70 (d, J=16.0 Hz, 1H), 3.25 (d, J=16.4 Hz, 1H), 1.46 (t, J=6.8 Hz, 2H), 0.86 (s, 3H), 0.11-0.00 (m, 4H). Stereoisomer 2: ¹H NMR (400 MHz, CD₃OD) δ 7.48 (dd, J=8.0, 8.0 Hz, 1H), 7.46 (dd, J=5.2, 3.2 Hz, 1H), 7.21-7.20 (m, 2H), 7.05-7.03 (m, 2H), 6.93 (dd, J=8.0, 8.0 Hz, 1H), 6.53-6.50 (m, 2H), 5.77 (dd, J=8.0, 1.6 Hz, 1H), 4.24 (t, J=6.8 Hz, 2H), 3.68 (d, J=16.0 Hz, 1H), 3.23 (d, J=16.4 Hz, 1H), 1.45 (t, J=6.8 Hz, 2H), 0.85 (s, 3H), 0.10-0.01 (m, 4H).

### Examples 11 and 12

### 3-(2-chlorophenyl)sulfanyl-6-[6-(4-fluoroanilino)-2-pyridyl]-1-methyl-6-(3-thienyl)piperidine-2,4-dione and 3-(2-chlorophenyl)sulfanyl-6-[6-(4-fluorophenoxy)-2-pyridyl]-1-methyl-6-(3-thienyl)piperidine-2,4-dione

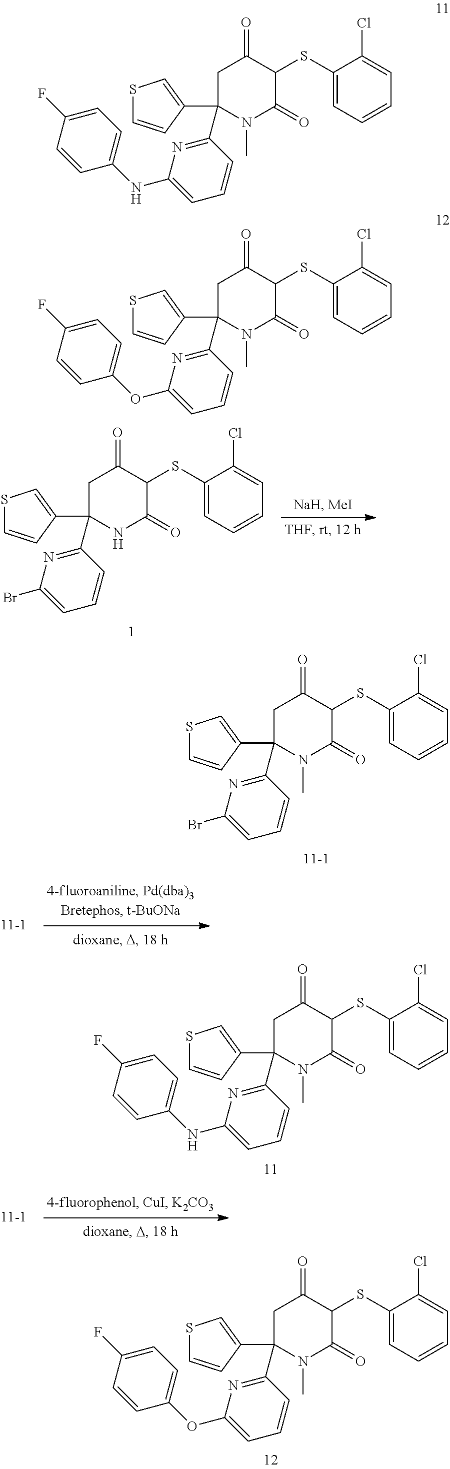

Step A: To a stirred solution of 6′-bromo-5-((2-chlorophenyl)thio)-4-hydroxy-2-(thiophen-3-yl)-2,3-dihydro-[2,2′-bipyridin]-6(1H)-one (1 g, 2 mmol) in anhydrous THF (20 mL) at 0° C. was added NaH (288 mg, 12 mmol). The reaction mixture was stirred at the same temperature for 0.5 hour, and then the reaction was added iodomethane (1.65 g, 12 mmol) and stirred at room temperature for 12 hours. The reaction was quenched with water, dried and concentrated. The crude residue was purified by silica gel chromatography eluting with a gradient of 10%-50% EtOAc/hexanes to afford 6′-bromo-5-((2-chlorophenyl)thio)-4-hydroxy-1-methyl-2-(thiophen-3-yl)-2,3-dihydro-[2,2′-bipyridin]-6(1H)-one (475 mg, 0.94 mol, 46% yield) as yellow solid.

Step B: 5-((2-Chlorophenyl)thio)-6′-((4-fluorophenyl)amino)-4-hydroxy-1-methyl-2-(thiophen-3-yl)-2,3-dihydro-[2,2′-bipyridin]-6(1H)-one was prepared in 8% yield according to the Example 4, Step A substituting cyclohexanamine for 4-fluoroaniline.

Step C: 5-((2-Chlorophenyl)thio)-6′-(4-fluorophenoxy)-4-hydroxy-1-methyl-2-(thiophen-3-yl)-2,3-dihydro-[2,2′-bipyridin]-6(1H)-one was prepared in 4% yield according to the Example 3, Step A 2-Chloro-4-fluoro-phenol for 4-fluorophenol and 6′-bromo-5-((2-chlorophenyl)thio)-4-hydroxy-2-(thiophen-3-yl)-2,3-dihydro-[2,2′-bipyridin]-6(1H)-one for 6′-bromo-5-((2-chlorophenyl)thio)-4-hydroxy-1-methyl-2-(thiophen-3-yl)-2,3-dihydro-[2,2′-bipyridin]-6(1H)-one.

Example 11: ¹H NMR (400 MHz, CD₃OD) δ 7.59-7.46 (m, 4H), 7.20 (dd, J=8.0, 1.2 Hz, 1H), 7.15 (dd, J=5.2, 1.6 Hz, 1H), 7.07 (dd, J=2.8, 1.6 Hz, 1H), 6.94-6.84 (m, 4H), 6.78 (dd, J=8.4, 0.8 Hz, 1H), 6.63 (dd, J=7.6, 0.4 Hz, 1H), 6.23 (dd, J=7.0, 1.6 Hz, 1H), 3.88 (d, J=16.8 Hz, 1H), 3.56 (d, J=16.8 Hz, 1H), 2.86 (s, 3H). LCMS M+1=537.8.

Example 12: ¹H NMR (400 MHz, CD₃OD) δ 7.88 (dd, J=8.4, 7.6 Hz, 1H), 7.49 (dd, J=5.2, 2.8 Hz, 1H), 7.23 (dd, J=8.0, 1.6 Hz, 1H), 7.09-6.95 (m, 8H), 6.89-6.83 (m, 2H), 6.04 (dd, J=8.0, 1.2 Hz, 1H), 3.57-3.46 (m, 2H), 2.65 (s, 3H). LCMS M+1=538.8.

### Example 13

### 3-(2-chlorophenyl)sulfanyl-6-(2-fluorophenyl)-1-methyl-6-(3-thienyl)piperidine-2,4-dione

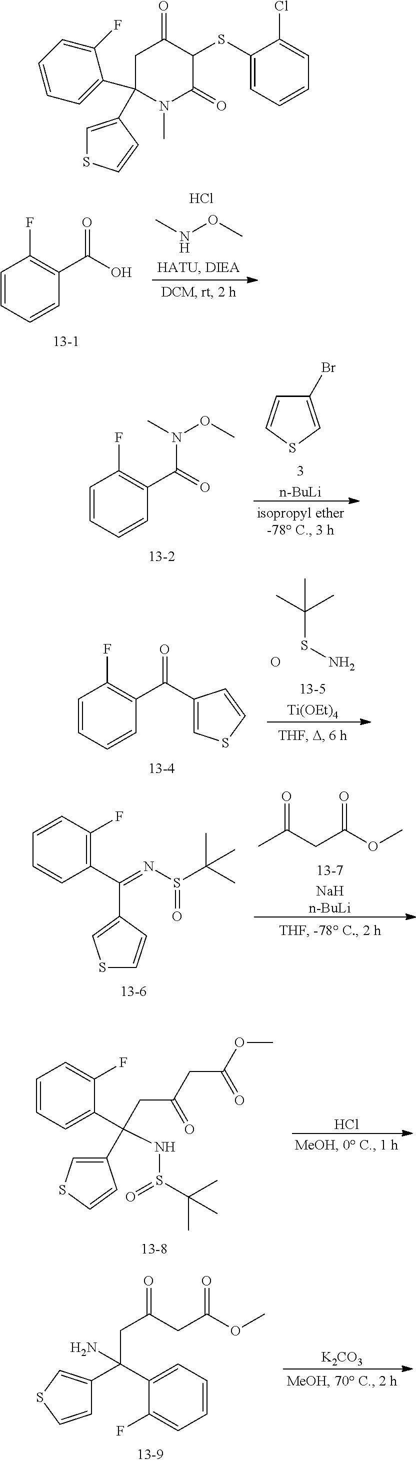

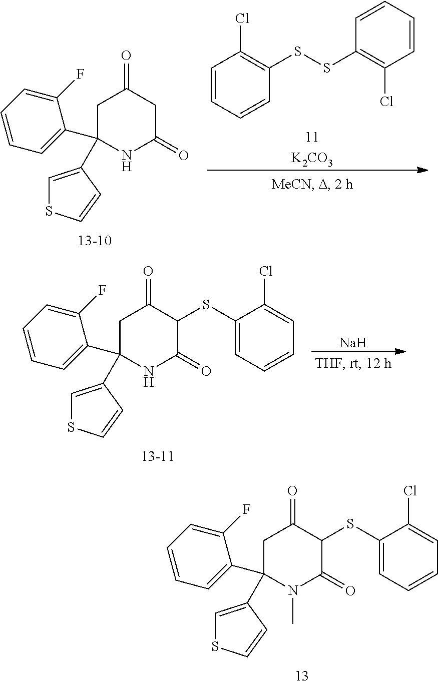

Step A: 2-Fluoro-N-methoxy-N-methylbenzamide was prepared in 73% yield according to the Example 1, Step A substituting 6-bromopicolinic acid for 2-fluorobenzoic acid.

Step B: (2-Fluorophenyl)(thiophen-3-yl)methanone was prepared in 99% yield according to the Example 1, Step B substituting 6-bromo-N-methoxy-N-methylpicolinamide for 2-fluoro-N-methoxy-N-methyl-benzamide.

Step C: (Z)—N-((2-Fluorophenyl)(thiophen-3-yl)methylene)-2-methylpropane-2-sulfinamide was prepared in 46% yield according to the Example 1, Step C substituting (6-bromopyridin-2-yl)(thiophen-3-yl)methanone for (2-fluorophenyl)(thiophen-3-yl)-methanone.

Step D: Methyl 5-(1,1-dimethylethylsulfinamido)-5-(2-fluorophenyl)-3-oxo-5-(thiophen-3-yl)pentanoate was prepared in 91% yield according to the Example 1, Step D substituting N-((6-bromopyridin-2-yl) (thiophen-3-yl)methylene)-2-methylpropane-2-sulfinamide for (Z)—N-((2-fluorophenyl) (thiophen-3-yl)methylene)-2-methyl-propane-2-sulfinamide.

Step E: Methyl 5-amino-5-(2-fluorophenyl)-3-oxo-5-(thiophen-3-yl)pentanoate was prepared in 33% yield according to the Example 1, Step E substituting methyl 5-(6-bromopyridin-2-yl)-5-(1,1-dimethylethylsulfinamido)-3-oxo-5-(thiophen-3-yl)pentanoate for methyl 5-(1,1-dimethylethylsulfinamido)-5-(2-fluorophenyl)-3-oxo-5-(thiophen-3-yl)pentanoate.

Step F: 6-(2-Fluorophenyl)-6-(thiophen-3-yl)piperidine-2,4-dione was prepared in 89% yield according to the Example 1, Step F substituting methyl-5-amino-5-(6-bromopyridin-2-yl)-3-oxo-5-(thiophen-3-yl)pentanoate for methyl 5-amino-5-(2-fluorophenyl)-3-oxo-5-(thiophen-3-yl)pentanoate.

Step G: 3-((2-Chlorophenyl)thio)-6-(2-fluorophenyl)-6-(thiophen-3-yl)piperidine-2,4-dione was prepared in 83% yield according to the Example 1, Step G substituting 6′-bromo-4-hydroxy-2-(thiophen-3-yl)-2,3-dihydro-[2,2′-bipyridin]-6(1H)-one for 6-(2-fluorophenyl)-6-(thiophen-3-yl)piperidine-2,4-dione.

Step H: 3-(2-chlorophenyl)sulfanyl-6-(2-fluorophenyl)-1-methyl-6-(3-thienyl)piperidine-2,4-dione was prepared in 30% yield according to the Example 11, Step B substituting 6′-bromo-4-hydroxy-2-(thiophen-3-yl)-2,3-dihydro-[2,2′-bipyridin]-6(1H)-one for 3-((2-chlorophenyl)thio)-6-(2-fluorophenyl)-6-(thiophen-3-yl)piperidine-2,4-dione. ¹H NMR (400 MHz, CD₃OD) δ 7.57 (d, J=4.0 Hz, 1H), 7.50-7.47 (m, 1H), 7.28-7.16 (m, 5H), 6.99-6.85 (m, 3H), 6.26 (dd, J=8.0, 4.0 Hz, 1H), 3.76-3.67 (m, 2H), 2.82 (s, 3H). LCMS M+1=445.9.

### Example 14

### 3-(2-chlorophenyl)sulfanyl-6-[5-(4-fluoroanilino)-2-hydroxy-phenyl]-6-(3-thienyl)piperidine-2,4-dione

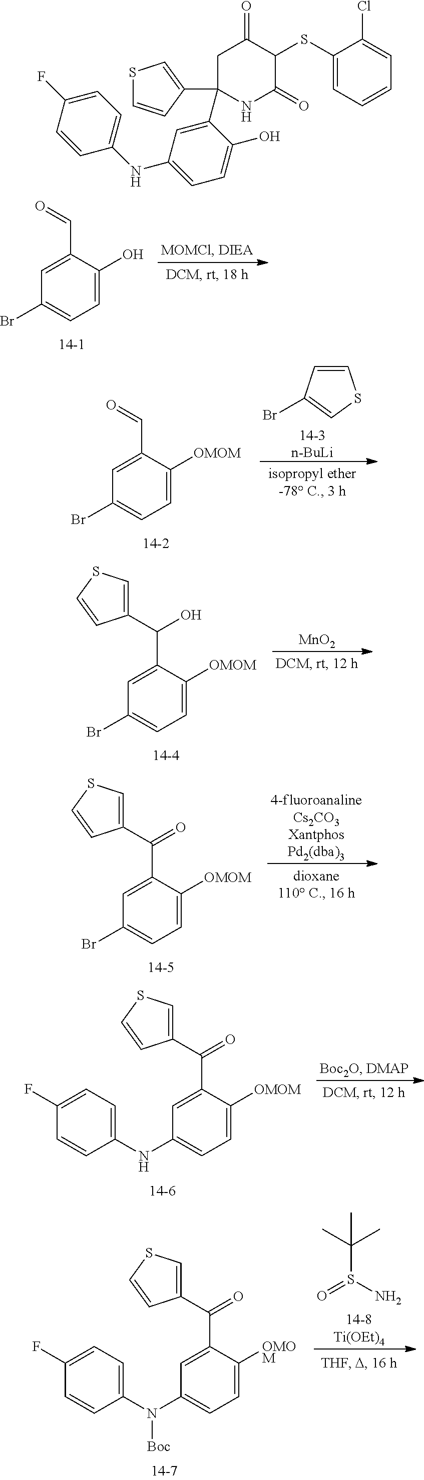

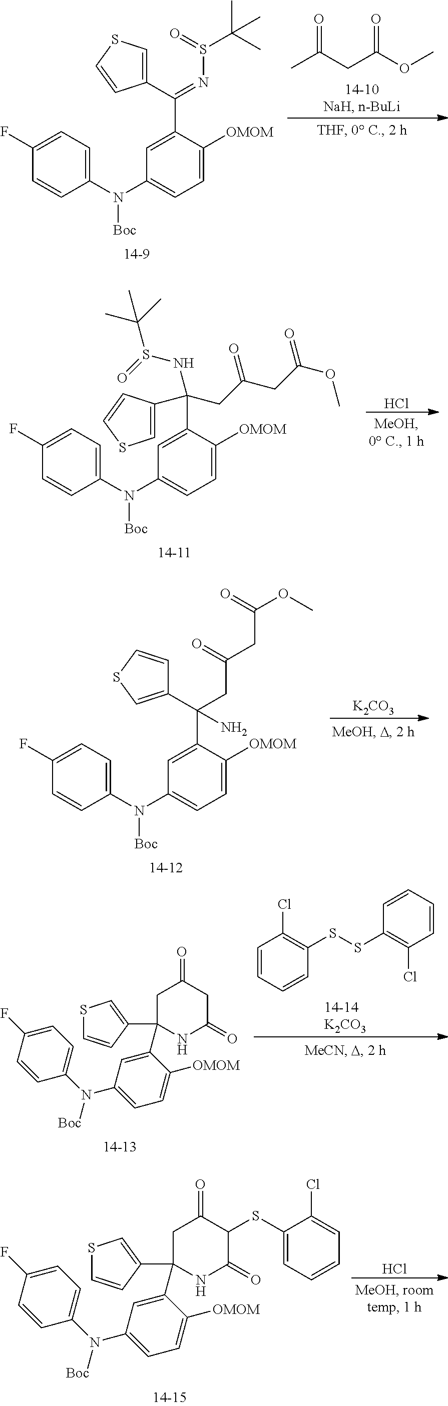

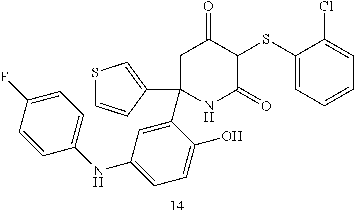 [SMILES: O=C1CC(c2ccsc2)(c2cc(Nc3ccc(F)cc3)ccc2O)NC(=O)C1Sc1ccccc1Cl]

Step A: Chloro(methoxy)methane (19.1 g, 0.23 mol) was added to a solution of 5-bromo-2-hydroxybenzaldehyde (30 g, 0.15 mol) and di-iso-propyl-ethylamine (38.5 g, 0.30 mol) at 0° C. in DCM. The mixture was warmed to ambient temperature and stirred for 18 hours. The reaction was quenched with water, dried over anhydrous Na₂SO₄ and concentrated to afford 5-bromo-2-(methoxymethoxy)benzaldehyde (30 g, 0.12 mol, 82% yield) as light color oil.

Step B: (5-Bromo-2-(methoxymethoxy)phenyl)(thiophen-3-yl)methanol was prepared in 77% yield according to the Example 2, Step A substituting 4-bromobenzaldehyde acid for 5-bromo-2-(methoxymethoxy)benzaldehyde.

Step C: (5-Bromo-2-(methoxymethoxy)phenyl)(thiophen-3-yl)methanol was prepared in 91% yield according to the Example 7, Step B substituting (4-bromophenyl)(thiophen-3-yl)methanol for (5-bromo-2-(methoxymethoxy)phenyl)(thiophen-3-yl)methanone.

Step D: A mixture of (5-bromo-2-(methoxymethoxy)phenyl)(thiophen-3-yl)methanone (10 g, 27.0 mmol), 4-fluoroaniline (10 g, 53.9 mmol), Xantphos (3.85 g, 5.39 mmol), Pd₂(dba)₃ (3.72 g, 2.7 mmol), Cs₂CO₃ (39.5 g, 80.9 mmol) and 1,4-dioxane (200 mL) was stirred at 110° C. for 16 hours. The reaction was cooled to room temperature, then filtered. The filtrate was concentrate under vacuum. The crude residue was purified by silica gel chromatography eluting with a gradient of 10%-50% EtOAc/hexanes to afford (5-((4-fluorophenyl)amino)-2-(methoxymethoxy)phenyl)(thiophen-3-yl)methanone (14 g, 39.2 mmol, 85% yield) as yellow solid.

Step E: A mixture of (5-((4-fluorophenyl)amino)-2-(methoxymethoxy)phenyl)(thiophen-3-yl)methanone (14 g, 39.2 mol), di-tert-butyl dicarbonate (16.9 g, 78.3 mmol), 4-dimethylaminopyridine (2.37 g, 19.6 mmol) and DCM (200 mL) was stirred at room temperature for 12 hours. The mixture was diluted with DCM (200 mL), washed with water (300 mL×2), brine, dried over Na₂SO₄ and concentrated. The crude was purified by silica gel chromatography eluting with a gradient of 10%-50% EtOAc/hexanes to afford tert-butyl (4-fluorophenyl)(4-(methoxymethoxy)-3-(thiophene-3-carbonyl)phenyl)carbamate (11.8 g, 25.8 mol, 91% yield) as yellow solid.

Step F: (Z)-tert-Butyl (3-(((tert-butylsulfinyl)imino)(thiophen-3-yl)methyl)-4-(methoxymethoxy)phenyl) (4-fluorophenyl)carbamate was prepared in 56% yield according to the Example 1, Step C substituting (6-bromopyridin-2-yl)(thiophen-3-yl)methanone for tert-butyl (4-fluorophenyl)(4-(methoxymethoxy)-3-(thiophene-3-carbonyl)phenyl)carbamate.

Step G: Methyl 5-(5-((tert-butoxycarbonyl)(4-fluorophenyl)amino)-2-(methoxymethoxy)phenyl)-5-(1,1-dimethylethylsulfinamido)-3-oxo-5-(thiophen-3-yl)pentanoate was prepared in 78% yield according to the Example 1, Step D substituting N-((6-bromopyridin-2-yl)(thiophen-3-yl)methylene)-2-methylpropane-2-sulfinamide for (Z)-tert-butyl (3-(((tert-butylsulfinyl)imino)(thiophen-3-yl)methyl)-4-(methoxymethoxy)phenyl) (4-fluorophenyl)carbamate.

Step H: Methyl 5-amino-5-(5-((tert-butoxycarbonyl)(4-fluorophenyl)amino)-2-(methoxymethoxy)phenyl)-3-oxo-5-(thiophen-3-yl)pentanoate was prepared in 86% yield according to the Example 1, Step E substituting methyl methyl 5-(6-bromopyridin-2-yl)-5-(1,1-dimethylethylsulfinamido)-3-oxo-5-(thiophen-3-yl)pentanoate for methyl 5-(5-((tert-butoxycarbonyl)(4-fluorophenyl)amino)-2-(methoxymethoxy)phenyl)-5-(1,1-dimethylethylsulfinamido)-3-oxo-5-(thiophen-3-yl)pentanoate.

Step I: tert-Butyl (3-(4,6-dioxo-2-(thiophen-3-yl)piperidin-2-yl)-4-(methoxymethoxy)phenyl)(4-fluorophenyl)carbamate was prepared in 93% yield according to the Example 1, Step F substituting methyl 5-amino-5-(6-bromopyridin-2-yl)-3-oxo-5-(thiophen-3-yl)pentanoate for methyl 5-amino-5-(5-((tert-butoxycarbonyl)(4-fluorophenyl)amino)-2-(methoxymethoxy)phenyl)-3-oxo-5-(thiophen-3-yl)pentanoate.

Step J: tert-Butyl (3-(5-((2-chlorophenyl)thio)-4,6-dioxo-2-(thiophen-3-yl)piperidin-2-yl)-4-(methoxymethoxy)phenyl)(4-fluorophenyl)carbamate was prepared in 70% yield according to the Example 1, Step G substituting 6′-bromo-4-hydroxy-2-(thiophen-3-yl)-2,3-dihydro-[2,2′-bipyridin]-6(1H)-one for tert-butyl (3-(4,6-dioxo-2-(thiophen-3-yl)piperidin-2-yl)-4-(methoxymethoxy)phenyl)(4-fluorophenyl)carbamate.

Step K: To a stirred solution of tert-butyl (3-(5-((2-chlorophenyl)thio)-4,6-dioxo-2-(thiophen-3-yl)piperidin-2-yl)-4-(methoxymethoxy)phenyl)(4-fluorophenyl)carbamate (600 mg, 0.88 mmol) in methanol (10 mL) was added HCl-MeOH (10 mL) in an ice bath. The reaction was stirred at room temperature for 1 hour. The mixture was neutralized by addition of 1 N NaOH. Then the mixture was extracted with EtOAc and water. The organic layer was dried over anhydrous NaSO₄ and concentrated. The crude was purified by prep-HPLC (formic acid) to afford 3-((2-chlorophenyl)thio)-6-(5-((4-fluorophenyl)amino)-2-hydroxyphenyl)-6-(thiophen-3-yl)piperidine-2,4-dione (150 mg, 0.28 mmol, 32% yield) as white solid. ¹H NMR (400 MHz, CD₃OD) δ 7.41 (dd, J=5.2, 5.2, 1H), 7.27-7.26 (m, 2H), 7.13-7.10 (m, 2H), 6.96-6.81 (m, 8H), 6.25 (dd, J=8.0, 1.6 Hz, 1H), 4.79-4.73 (m, 1H), 3.79 (d, J=17.2 Hz, 1H), 3.43 (d, J=16.8 Hz, 1H). LCMS M+1=538.8.

### Example 15

### 3-(2-chlorophenyl)sulfanyl-6-(1H-indol-4-yl)-6-(3-thienyl)piperidine-2,4-dione

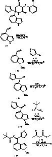

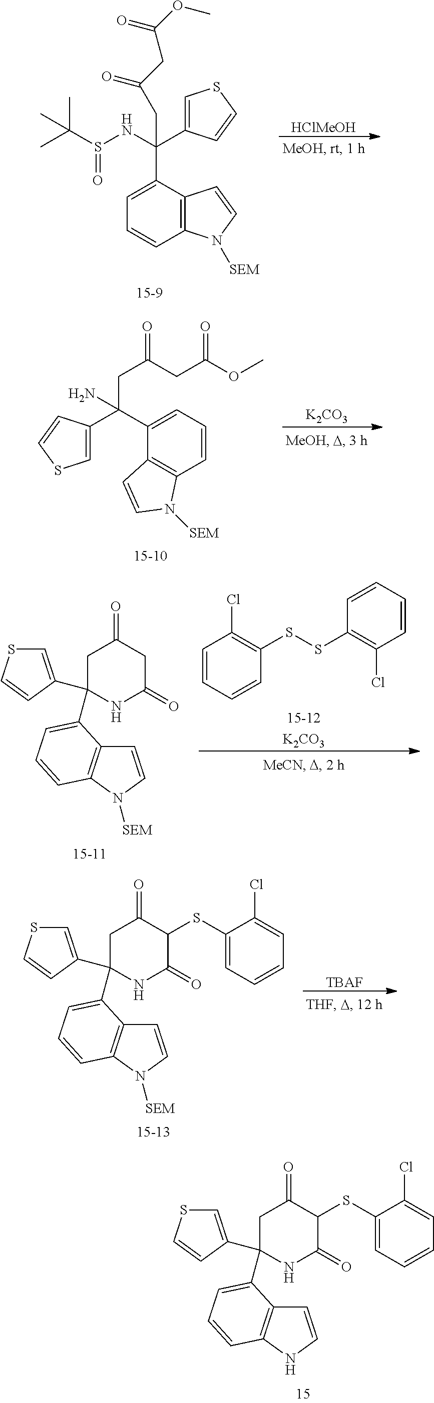

Step A: 1H-Indole-4-carbaldehyde (10 g, 69.0 mmol) was added to a suspension of NaH (2.0 g, 82.6 mmol) in anhydrous THF (150 mL) at 0° C. The resultant suspension was stirred at 0° C. for 30 minutes, followed by addition of 2-(trimethylsilyl) ethoxymethyl chloride (13.8 g, 82.6 mmol). The reaction mixture was stirred at room temperature for 3 hours. The reaction was quenched with water, dried over anhydrous Na₂SO₄ and the filtrate was concentrated under vacuum. The crude residue was purified by silica gel chromatography eluting with a gradient of 10%-30% EtOAc/hexanes to afford 1-((2-(trimethylsilyl)ethoxy)methyl)-1H-indole-4-carbaldehyde (817 g, 61.4 mmol, 89% yield) as dark yellow solid.

Step B: Thiophen-3-yl(1-((2-(trimethylsilyl)ethoxy)methyl)-1H-indol-4-yl)methanol was prepared in 68% yield according to the Example 2, Step A substituting 4-bromobenzaldehyde acid for 1-((2-(trimethylsilyl)ethoxy)methyl)-1H-indole-4-carbaldehyde.

Step C: Thiophen-3-yl(1-((2-(trimethylsilyl)ethoxy)methyl)-1H-indol-4-yl)methanone was prepared in 94% yield according to the Example 7, Step B substituting (4-bromophenyl)(thiophen-3-yl)methanol for thiophen-3-yl(1-((2-(trimethylsilyl)ethoxy)methyl)-1H-indol-4-yl)methanol.

Step D: (E)-2-Methyl-N-(thiophen-3-yl(1-((2-(trimethylsilyl)ethoxy)methyl)-1H-indol-4-yl)methylene)propane-2-sulfinamide was prepared in 64% yield according to the Example 1, Step C substituting (6-bromopyridin-2-yl)(thiophen-3-yl)methanone for thiophen-3-yl(1-((2-(trimethylsilyl)ethoxy)methyl)-1H-indol-4-yl)methanone.

Step E: Methyl 5-(1,1-dimethylethylsulfinamido)-3-oxo-5-(thiophen-3-yl)-5-(1-((2-(trimethylsilyl)ethoxy)methyl)-1H-indol-4-yl)pentanoate was prepared in 88% yield according to the Example 1, Step D substituting N-((6-bromopyridin-2-yl) (thiophen-3-yl)methylene)-2-methylpropane-2-sulfinamide for (E)-2-methyl-N-(thiophen-3-yl(1-((2-(trimethylsilyl)ethoxy)methyl)-1H-indol-4-yl)methylene)propane-2-sulfinamide.

Step F: Methyl 5-amino-3-oxo-5-(thiophen-3-yl)-5-(1-((2-(trimethylsilyl)ethoxy)methyl)-1H-indol-4-yl)pentanoate was prepared in 65% yield according to the Example 1, Step E substituting methyl methyl 5-(6-bromopyridin-2-yl)-5-(1,1-dimethylethylsulfinamido)-3-oxo-5-(thiophen-3-yl)pentanoate for methyl 5-(1,1-dimethylethylsulfinamido)-3-oxo-5-(thiophen-3-yl)-5-(1-((2-(trimethylsilyl)ethoxy)methyl)-1H-indol-4-yl)pentanoate.

Step G: 6-(Thiophen-3-yl)-6-(1-((2-(trimethylsilyl)ethoxy)methyl)-1H-indol-4-yl)piperidine-2,4-dione was prepared in 43% yield according to the Example 1, Step F substituting methyl 5-amino-5-(6-bromopyridin-2-yl)-3-oxo-5-(thiophen-3-yl)pentanoate for methyl 5-amino-3-oxo-5-(thiophen-3-yl)-5-(1-((2-(trimethylsilyl)ethoxy)methyl)-1H-indol-4-yl)-pentanoate.

Step H: 3-((2-Chlorophenyl)thio)-6-(thiophen-3-yl)-6-(1-((2-(trimethylsilyl)ethoxy)methyl)-1H-indol-4-yl)piperidine-2,4-dione was prepared in 59% yield according to the Example 1, Step G substituting 6′-bromo-4-hydroxy-2-(thiophen-3-yl)-2,3-dihydro-[2,2′-bipyridin]-6(1H)-one for 6-(thiophen-3-yl)-6-(1-((2-(trimethylsilyl)ethoxy)methyl)-1H-indol-4-yl)piperidine-2,4-dione.

Step I: To a stirred solution of 3-((2-chlorophenyl)thio)-6-(thiophen-3-yl)-6-(1-((2-(trimethylsilyl)ethoxy)methyl)-1H-indol-4-yl)piperidine-2,4-dione (250 mg, 0.43 mmol) in THF (4 mL) was added TBAF (4 mL, 1M in THF). The reaction was heated at 80° C. for 12 hours. After cooling to room temperature, the reaction mixture was diluted with EtOAc (20 mL), washed with water and concentrated under vacuum. The crude was purified by preparative HPLC (formic acid) to afford 3-((2-chlorophenyl)thio)-6-(1H-indol-4-yl)-6-(thiophen-3-yl)piperidine-2,4-dione (24 mg, 0.05 mmol, 12% yield) as white solid. ¹H NMR (400 MHz, CD₃OD) δ 7.58 (dd, J=8.0, 8.0 Hz, 1H), 7.43-7.41 (m, 2H), 7.27-7.19 (m, 3H), 7.05 (dd, J=8.0, 8.0 Hz, 1H), 6.91-6.88 (m, 1H), 6.77-6.75 (m, 2H), 6.53 (d, J=8.4 Hz, 1H), 6.08 (dd, J=8.0, 4.0 Hz, 1H), 3.88 (d, J=16.0 Hz, 1H), 3.50 (d, J=16.0 Hz, 1H). LCMS M+1=452.8.

### Example 16

### 3-(2-chlorophenyl)sulfanyl-6-[6-[2-(oxetan-3-yl)ethoxy]-2-pyridyl]-6-(3-thienyl)piperidine-2,4-di one

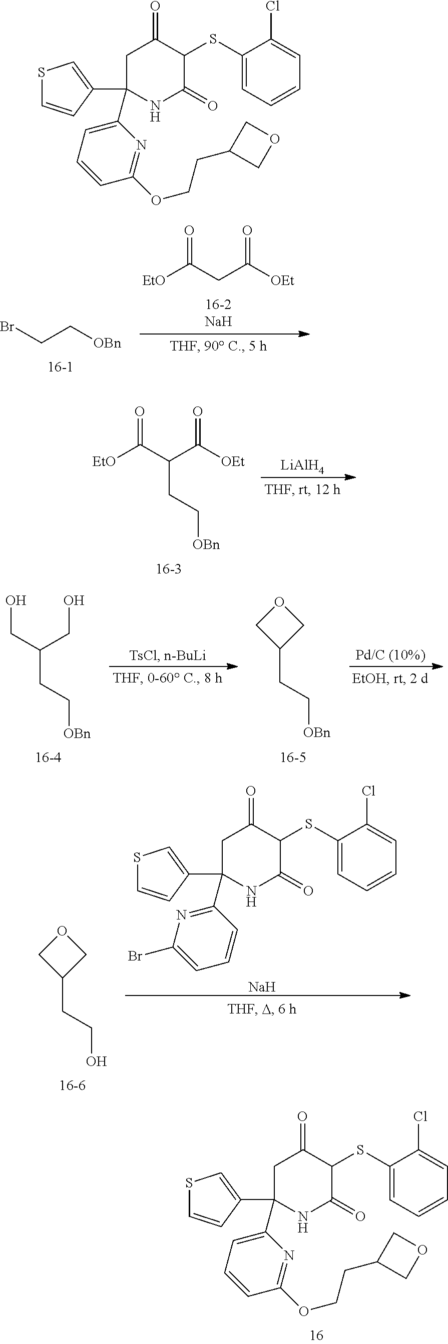

Step A: To a suspension of NaH (688 mg, 27.8 mmol) in THF (80 mL) was added diethyl malonate (7.45 g, 46.5 mmol) dropwise. Then ((2-bromoethoxy)methyl)benzene (5 g, 23.2 mmol) was added. The reaction was heated to 90° C. for 5 hours. After cooling to room temperature, the mixture was diluted with EtOAc (50 mL), washed with water (50 mL×2), dried over anhydrous Na₂SO₄ and concentrated under vacuum. The crude residue was purified by silica gel chromatography eluting with a gradient of 10%-30% EtOAc/hexanes to afford diethyl 2-(2-(benzyloxy)ethyl)malonate (6.6 g, 22.5 mmol, 81% yield) as a colorless oil.

Step B: To a suspension of LiAlH₄ (1.71 g, 45.0 mmol) in anhydrous THF (80 mL) was added diethyl 2-(2-(benzyloxy)ethyl)malonate (6.6 g, 22.5 mmol) dropwise in an ice bath. The reaction was warmed to room temperature and stirred for 12 hours. The reaction was quenched with water, diluted with EtOAc (50 mL), washed with water (50 mL×2), dried over anhydrous Na₂SO₄ and concentrated under vacuum. The crude residue was purified by silica gel chromatography eluting with a gradient of 10%-30% EtOAc/hexanes to afford 2-(2-(benzyloxy)ethyl)propane-1,3-diol (2.2 g, 10.6 mmol, 47% yield) as a colorless oil.

Step C: To solution of 2-(2-(benzyloxy)ethyl)propane-1,3-diol (2.2 g, 10.6 mmol) in THF (20 mL) was added n-BuLi (4.2 mL, 10.6 mmol) in an ice bath. The mixture was stirred at 0° C. for 30 minutes, then TsCl (404 mg, 2.12 mmol) was added. The reaction mixture was stirred at 0° C. for 1 hour and then n-BuLi (4.2 mL, 10.6 mmol) was added. The reaction mixture was stirred at 60° C. for 6 hours, then cooled to room temperature. The mixture was diluted with EtOAc (30 mL), washed with water (50 mL×2), dried over anhydrous Na₂SO₄ and concentrated under vacuum. The crude residue was purified by silica gel chromatography eluting with a gradient of 0%-15% EtOAc/hexanes to afford 3-(2-(benzyloxy)ethyl)oxetane (550 mg, 2.86 mmol, 27% yield) as a colorless oil.

Step D: A mixture of 3-(2-(benzyloxy)ethyl)oxetane (550 mg, 2.86 mmol), Pd/C (350 mg) and ethanol (5 mL) was stirred at room temperature under hydrogen atmosphere for 2 days. The mixture was filtered and the filtrate was concentrate to afford 2-(oxetan-3-yl)ethanol (200 mg, 1.96 mmol, 66% yield) as a colorless oil.

Step E:

5-((2-Chlorophenyl)thio)-4-hydroxy-6′-(2-(oxetan-3-yl)ethoxy)-2-(thiophen-3-yl)-2,3-dihydro-[2,2′-bipyridin]-6(1H)-one was prepared in 35% yield according to the Example 2, Step A substituting propan-2-ol for 2-(oxetan-3-yl)ethanol. ¹H NMR (400 MHz, CD₃OD) δ 7.70 (dd, J=8.0, 8.0 Hz, 1H), 7.40 (dd, J=2.8, 2.8 Hz, 1H), 7.27 (d, J=2.8 Hz, 1H), 7.17-7.14 (m, 3H), 6.88 (dd, J=8.0, 8.0 Hz, 1H), 6.75-6.73 (m, 2H), 6.00 (dd, J=9.6, 1.6 Hz, 1H), 4.38-4.28 (m, 2H), 3.83-3.69 (m, 5H), 3.43-3.41 (m, 1H), 2.71-2.67 (m, 1H), 2.06-2.02 (m, 1H), 1.75-1.71 (m, 1H). LCMS M+1=514.9.

### Example 17

### 3-(2-chlorophenyl)sulfanyl-6-[6-[[3-(hydroxymethyl)phenyl]methyl]-2-pyridyl]-6-(3-thienyl)piperidine-2,4-dione

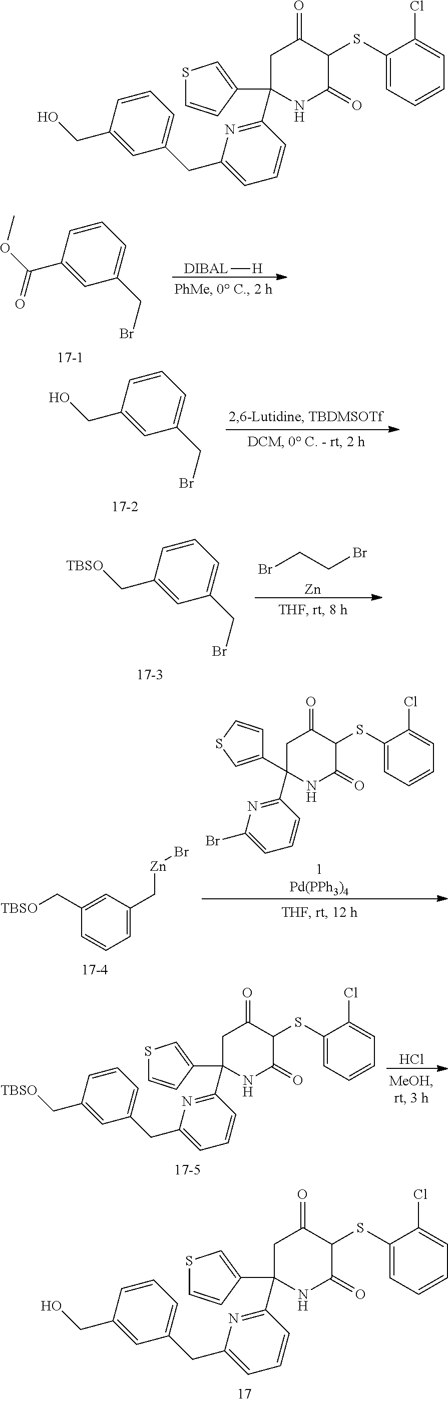

Step A: To a stirred solution of methyl 3-(bromomethyl)benzoate (5 g, 21.8 mmol) in toluene (50 mL) was added DABAL-H (43.6 ml, 43.6 mmol) in an ice bath. The reaction was stirred at 0° C. for 2 hours. The mixture was quenched with 1 N HCl, extracted with EtOAc and water. The organic layer was dried over anhydrous Na₂SO₄ and concentrated to afford (3-(bromomethyl)phenyl)methanol (4.0 g, 19.9 mmol, 91% yield) as a colorless oil.

Step B: A mixture of (3-(bromomethyl)phenyl)methanol (2.0 g, 10.0 mmol), 2,6-lutidine (2.13 g, 19.9 mmol), tert-butyl dimethylsilyl trifluoromethanosulfonate (3.1 g, 14.9 mmol) and DCM (30 mL) was stirred at room temperature for 2 hours. The reaction was quenched with water (20 mL), extracted with DCM. The organic layer was dried over anhydrous Na₂SO₄ and concentrated. The crude residue was purified by chromatography on silica gel (petroleum ether/EtOAc=20/1) to afford (3-(bromomethyl)phenyl)methanol (2.8 g, 8.9 mmol, 89% yield) as a colorless oil.

Step C: To a mixture of zinc powder (408 mg, 6.3 mol) in anhydrous THF (30 mL) was added 1,2-dibromoethane (107 mg, 0.57 mmol) and ((3-(bromomethyl)benzyl)oxy)(tert-butyl)dimethylsilane (1.8 g. 5.7 mmol) under nitrogen atmosphere. The mixture was stirred at room temperature for 8 hours. The reaction solution was used in next step directly.

Step D: To a stirred solution of 6′-bromo-5-((2-chlorophenyl)thio)-4-hydroxy-2-(thiophen-3-yl)-2,3-dihydro-[2,2′-bipyridin]-6(1H)-one (example 1, 300 mg, 0.61 mmol) and Pd(PPh₃)₄ (69 mg, 0.06 mmol) in THF (1 mL) was added (3-(((tert-butyldimethylsilyl)oxy)methyl)benzyl)zinc(II) bromide (5.3 mL, 3.04 mmol). The mixture was stirred at room temperature for 12 hours. The reaction was quenched with water, then filtered over Celite. The filtrate was concentrated under vacuum and the crude residue was purified by preparative HPLC (formic acid) to afford 6′-(3-(((tert-butyldimethylsilyl)oxy)methyl)benzyl)-5-((2-chlorophenyl)thio)-4-hydroxy-2-(thiophen-3-yl)-2,3-dihydro-[2,2′-bipyridin]-6(1H)-one (80 mg, 0.12 mmol, 20% yield) as white solid.

Step E: To a stirred solution of 6′-(3-(((tert-butyldimethylsilyl)oxy)methyl)benzyl)-5-((2-chlorophenyl)thio)-4-hydroxy-2-(thiophen-3-yl)-2,3-dihydro-[2,2′-bipyridin]-6(1H)-one (80 mg, 0.12 mmol) in MeOH (5 mL) was added HCl-MeOH (5 mL) in an ice bath. The mixture was stirred at 0° C. for 1 hour. The reaction was added water, then filtered and washed with water. The solid was dried to afford 5-((2-chlorophenyl)thio)-4-hydroxy-6′-(3-(hydroxymethyl)benzyl)-2-(thiophen-3-yl)-2,3-dihydro-[2,2′-bipyridin]-6(1H)-one (50 mg, 0.09 mmol, 76% yield) as a white solid. Mixture of diastereoisomers: ¹H NMR (400 MHz, CD₃OD) δ 7.74 (dd, J=8.0, 8.0 Hz, 1H), 7.42-7.40 (m, 2H), 7.22-7.17 (m, 6H), 7.11-7.09 (m, 2H), 6.85 (dd, J=8.0, 8.0 Hz, 1H), 6.51 (dd, J=8.0, 8.0 Hz, 1H), 5.78 (dd, J=8.4, 1.6 Hz, 1H), 4.47 (s, 2H), 4.17 (s, 2H), 4.00 (d, J=16.4 Hz, 1H), 3.47 (d, J=16.4 Hz, 1H). LCMS M+1=534.9. Stereoisomer 1: ¹H NMR (400 MHz, CD₃OD) δ 7.75 (dd, J=8.0, 8.0 Hz, 1H), 7.44-7.42 (m, 2H), 7.31-7.14 (m, 8H), 6.85 (dd, J=8.0, 8.0 Hz, 1H), 6.56 (dd, J=8.0, 8.0 Hz, 1H), 5.87 (d, J=8.0 Hz, 1H), 4.51 (s, 2H), 4.20 (s, 2H), 3.92 (d, J=16.4 Hz, 1H), 3.49 (d, J=16.4 Hz, 1H). Stereoisomer 2: ¹H NMR (400 MHz, CD₃OD) δ 7.74 (dd, J=8.0, 8.0 Hz, 1H), 7.44-7.41 (m, 2H), 7.16-7.13 (m, 8H), 6.87 (dd, J=8.0, 8.0 Hz, 1H), 6.56 (dd, J=8.0, 8.0 Hz, 1H), 5.87 (d, J=8.0 Hz, 1H), 4.51 (s, 2H), 4.20 (s, 2H), 3.91 (d, J=16.4 Hz, 1H), 3.49 (d, J=16.0 Hz, 1H).

### Example 18

### 3-(2-chlorophenyl)sulfanyl-6-[6-[2-(1H-pyrazol-4-yl)phenoxy]-2-pyridyl]-6-(3-thienyl)piperidine-2,4-dione

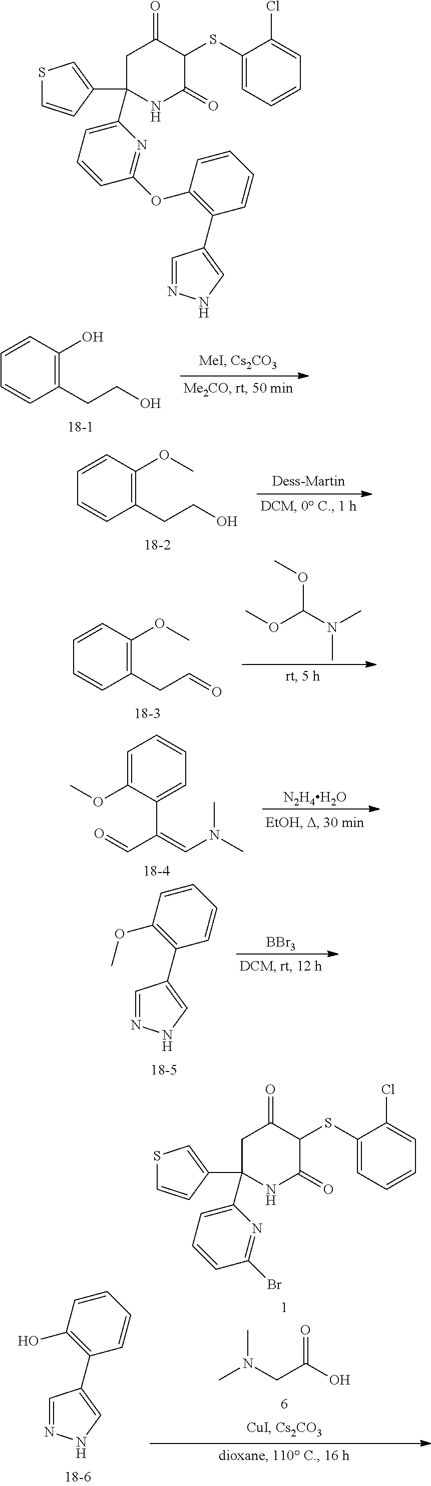

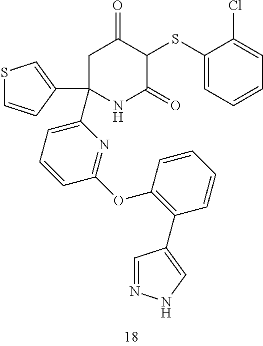 [SMILES: O=C1CC(c2ccsc2)(c2cccc(Oc3ccccc3-c3cn[nH]c3)n2)NC(=O)C1Sc1ccccc1Cl]

Step A: To a stirred suspension of 2-(2-hydroxyethyl)phenol (5 g, 36.2 mmol) and Cs₂CO₃ (38.9 g, 108.7 mmol) in acetone (100 mL) was added iodomethane (6.2 g, 43.4 mmol) in an ice bath. The reaction mixture was stirred at 0° C. for 50 minutes. The mixture was filtered, the filtrate was concentrated under vacuum. The crude materials were extracted with EtOAc and water. The organic layer was dried over anhydrous Na₂SO₄ and concentrated to afford 2-(2-methoxyphenyl)ethanol (4.5 g, 29.6 mmol, 82% yield) as a yellow solid.

Step B: To a stirred solution of 2-(2-methoxyphenyl)ethanol (4.5 g, 29.6 mmol) in DCM (80 mL) was added Dess-Martin reagent (51.1 g, 35.5 mmol) in an ice bath. The reaction mixture was stirred at 0° C. for 1 hour. The mixture was diluted with DCM (100 mL), washed with saturated NaHCO₃ (100 mL×2), brine, dried over anhydrous Na₂SO₄ and concentrated. The crude residue was purified by silica gel chromatography eluting with a gradient of 10%-50% EtOAc/hexanes to afford 2-(2-methoxyphenyl)acetaldehyde (2.5 g, 16.7 mmol, 56% yield) as yellow oil.

Step C: A mixture of 2-(2-methoxyphenyl)acetaldehyde (2.5 g, 16.7 mmol) and 1,1-dimethoxy-N,N-dimethylmethanamine (5 mL) was stirred at room temperature for 5 hours. The mixture was diluted with DCM (30 mL), washed with saturated NaHCO₃ (20 mL×2), brine, dried over anhydrous Na₂SO₄ and concentrated. The crude residue was purified by silica gel chromatography eluting with a gradient of 10%-50% EtOAc/hexanes to afford (E)-3-(dimethylamino)-2-(2-methoxyphenyl)acrylaldehyde (350 mg, 1.7 mmol, 12% yield) as yellow solid.

Step D: A mixture of (E)-3-(dimethylamino)-2-(2-methoxyphenyl)acrylaldehyde (350 mg, 1.7 mmol,), hydrazine hydrate (2 mL) and ethanol (5 mL) was heated to 80° C. for 30 minutes. The mixture was diluted with DCM 10 mL), washed with saturated NaHCO₃ (10 mL×2), brine, dried over anhydrous Na₂SO₄ and concentrated to afford 4-(2-methoxyphenyl)-1H-pyrazole (260 mg, 1.5 mmol, 88% yield) as yellow solid.

Step E: To a stirred solution of 4-(2-methoxyphenyl)-1H-pyrazole (260 mg, 1.5 mmol) in DCM (5 mL) was added boron tribromide (750 mg, 3.0 mmol) in an ice bath. The reaction mixture was stirred at 0° C. for 12 hours. The mixture was diluted with DCM (20 mL), washed with saturated NaHCO₃ (20 mL×2), brine, dried over anhydrous Na₂SO₄ and concentrated to afford 2-(1H-pyrazol-4-yl)phenol (200 mg, 1.25 mmol, 84% yield) as yellow oil.

Step F: 6′-(2-(1H-Pyrazol-4-yl)phenoxy)-5-((2-chlorophenyl)thio)-4-hydroxy-2-(thiophen-3-yl)-2,3-dihydro-[2, 2′-bipyridin]-6(1H)-one was prepared in 3% yield according to the Example 3, Step A 2-Chloro-4-fluoro-phenol for 2-(1H-pyrazol-4-yl)phenol. ¹H NMR (400 MHz, (CD₃)₂SO) δ 11.75 (s, 1H), 9.92 (s, 1H), 9.08 (s, 1H), 9.64 (s, 1H), 8.30 (s, 1H), 8.07 (dd, J=8.0, 8.0 Hz, 1H), 7.90 (d, J=8.4 Hz, 1H), 7.59-7.53 (m, 3H), 7.40 (s, 1H), 7.23-7.21 (m, 2H), 7.08 (dd, J=8.0, 4.0 Hz, 1H), 6.93-6.84 (m, 3H), 6.60 (dd, J=8.0, 8.0 Hz, 1H), 5.85 (d, J=8.4 Hz, 1H), 4.04 (d, J=16.0 Hz, 1H), 3.43 (d, J=16.4 Hz, 1H). LCMS M+1=573.1.

### Example 19

### 3-(2-chlorophenyl)sulfanyl-6-(5-chloro-3-thienyl)-6-[6-(4-fluorophenoxy)-2-pyridyl]piperidine-2,4-dione

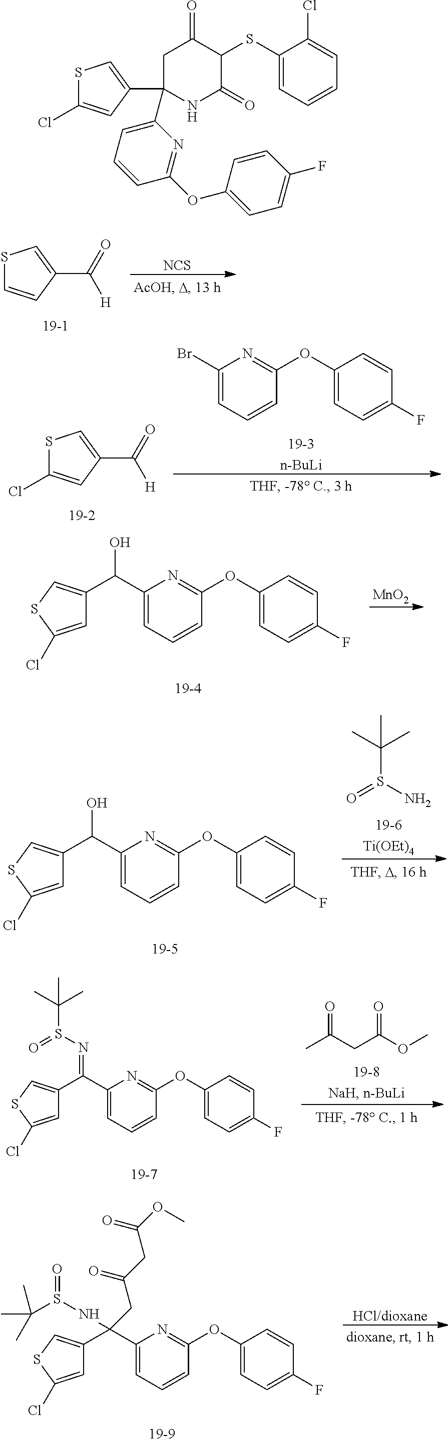

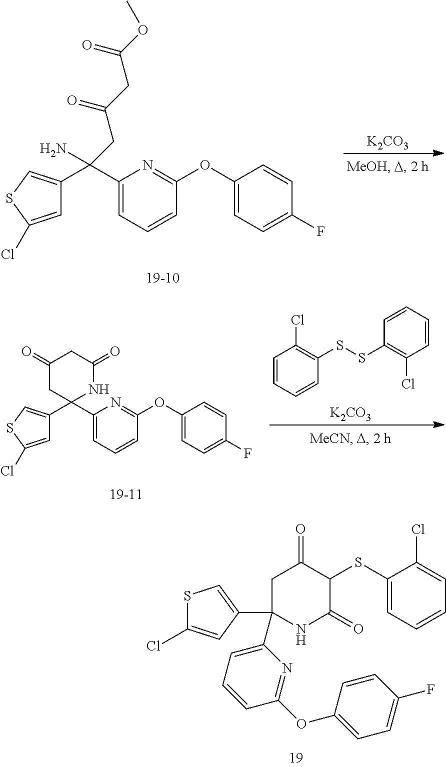

Step A: To a solution of thiophene-3-carbaldehyde (20.0 g, 178.3 mmol) and N-chlorosuccinimide (23.8 g, 178.3 mmol) in AcOH (180 mL) was stirred at 110° C. for 4 hours. After the completion of reaction, the solution was cooled to room temperature, and then was diluted with EtOAc (120 mL), washed with H₂O (100 mL×3), saturated NaHCO₃ (50 mL×2), brine, dried over anhydrous Na₂SO₄ and concentrated to afford 5-chlorothiophene-3-carboxylic acid (8.0 g, 54.6 mmol, 31% yield) as yellow solid, which was used directly in the next step without further purification.

Step B: (5-Chlorothiophen-3-yl)(6-(4-fluorophenoxy)pyridin-2-yl)methanol was prepared in 50% yield according to the Example 7, Step A substituting 4-bromobenzaldehyde for 5-chlorothiophene-3-carbaldehyde and 3-bromothiophene for 2-bromo-6-(4-fluorophenoxy)pyridine

Step C: (5-Chlorothiophen-3-yl)(6-(4-fluorophenoxy)pyridin-2-yl)methanone was prepared in 79% yield according to the Example 7, Step B substituting (4-bromophenyl)(thiophen-3-yl)methanone for (5-chlorothiophen-3-yl)(6-(4-fluorophenoxy)pyridine-2-yl)methanone.

Step D: (E)-N-((5-Chlorothiophen-3-yl)(6-(4-fluorophenoxy)pyridin-2-yl)methylene)-2-methylpropane-2-sulfinamide was prepared in 74% yield according to the Example 7, Step C substituting (E)-N-((4-bromophenyl) (thiophen-3-yl)methylene)-2-methylpropane-2-sulfinamide for (E)-N-((5-chlorothiophen-3-yl)(6-(4-fluorophenoxy)pyridin-2-yl)methylene)-2-methylpropane-2-sulfinamide

Step E: Methyl 5-(5-chlorothiophen-3-yl)-5-(1,1-dimethylethylsulfinamido)-5-(6-(4-fluoro phenoxy)pyridin-2-yl)-3-oxopentanoate was prepared in 86% yield according to the Example 7, Step D substituting methyl 5-(4-bromophenyl)-5-(1,1-dimethylethylsulfinamido)-3-oxo-5-(thiophen-3-yl)pentanoate for methyl 5-(5-chlorothiophen-3-yl)-5-(1,1-dimethylethylsulfinamido)-5-(6-(4-fluoro phenoxy)pyridin-2-yl)-3-oxopentanoate

Step F: Methyl 5-amino-5-(5-chlorothiophen-3-yl)-5-(6-(4-fluorophenoxy)pyridin-2-yl)-3-oxopentanoate was prepared in 49% yield according to the Example 7, Step E substituting methyl 5-amino-5-(4-bromophenyl)-3-oxo-5-(thiophen-3-yl)pentanoate for methyl 5-amino-5-(5-chlorothiophen-3-yl)-5-(6-(4-fluorophenoxy)pyridin-2-yl)-3-oxopentanoate

Step G: 6-(5-Chlorothiophen-3-yl)-6-(6-(4-fluorophenoxy)pyridin-2-yl)piperidine-2,4-dione was prepared in 57% yield according to the Example 7, Step F substituting 6-(4-bromophenyl)-4-hydroxy-6-(thiophen-3-yl)-5,6-dihydropyridin-2(1H)-one for 6-(5-chlorothiophen-3-yl)-6-(6-(4-fluorophenoxy)pyridin-2-yl)piperidine-2,4-dione.

Step H: 5-((2-Chlorophenyl)thio)-2-(5-chlorothiophen-3-yl)-6′-(4-fluorophenoxy)-4-hydroxy-2,3-dihydro-[2,2′-bipyridin]-6(1H)-one was prepared in 5.4% yield according to the Example 7, Step G substituting 6-(4-bromophenyl)-3-((2-chlorophenyl)thio)-4-hydroxy-6-(thiophen-3-yl)-5,6-dihydropyridin-2(111H)-one for 5-((2-chlorophenyl)thio)-2-(5-chlorothiophen-3-yl)-6′-(4-fluorophenoxy)-4-hydroxy-2,3-dihydro-[2,2′-bipyridin]-6(1H)-one. Mixture of diastereoisomers: ¹H NMR (400 MHz, CD₃OD) δ 7.89 (dd, J=7.6, 7.6 Hz, 1H), 7.31 (d, J=8.0 Hz, 1H), 7.21 (d, J=7.8 Hz, 1H), 7.12-7.08 (m, 4H), 7.01-6.89 (m, 3H), 6.98-6.89 (m, 2H), 6.07 (dd, J=8.0, 1.2 Hz, 1H), 3.54 (d, J=16.0 Hz, 1H), 3.25 (d, J=16.0 Hz, 1H). LCMS M+1=558.9. Stereoisomer 1: ¹H NMR (400 MHz, CD₃OD) δ 7.89 (dd, J=7.6, 7.6 Hz, 1H), 7.33 (d, J=8.0 Hz, 1H), 7.22 (d, J=7.8 Hz, 1H), 7.10-7.07 (m, 4H), 7.01-6.89 (m, 3H), 6.89-6.81 (m, 2H), 6.07 (d, J=6.8 Hz, 1H), 3.55 (d, J=16.0 Hz, 1H), 3.28 (d, J=16.0 Hz, 1H). Stereoisomer 2: ¹H NMR (400 MHz, CD₃OD) δ 7.89 (dd, J=7.6, 7.6 Hz, 1H), 7.33 (d, J=8.0 Hz, 1H), 7.12 (d, J=7.8 Hz, 1H), 7.10-7.08 (m, 4H), 7.01-6.99 (m, 3H), 6.89-6.81 (m, 2H), 6.08 (d, J=6.8 Hz, 1H), 3.54 (d, J=16.0 Hz, 1H), 3.28 (d, J=16.0 Hz, 1H).

### Example 20

### 3-(2-chlorophenyl)sulfanyl-6-[6-[2-(2,2-difluorocyclopropyl)ethoxy]-2-pyridyl]-6-(3-thienyl)piperidine-2,4-dione

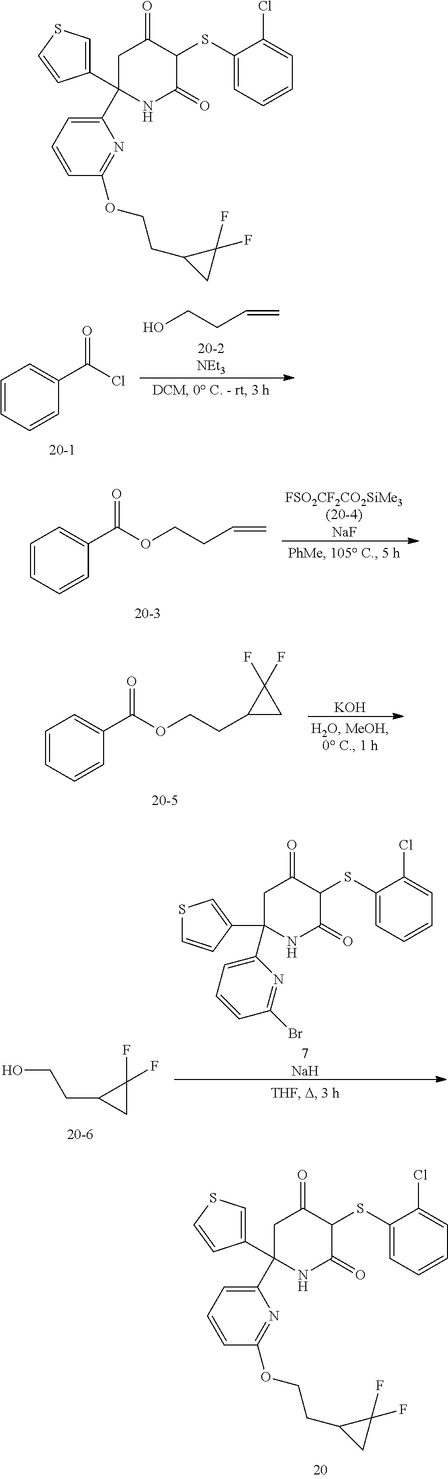

Step A: Benzyl chloride (2.0 g, 14.2 mmol) was added dropwise to a solution of but-3-en-1-ol (1.2 g, 17.1 mmol) and Et₃N (2.9 g, 28.5 mmol) at 0° C. in DCM (35 mL) The reaction mixture was then warmed to ambient temperature and stirred for 3 hours. The reaction mixture was quenched with saturated aqueous NH₄Cl (10 mL). The organic layer was washed with saturated NaHCO₃ solution (5 mL×2), brine, dried over anhydrous Na₂SO₄ and concentrated to afford crude product which was purified by silica gel chromatography eluting with 20% EtOAc/hexanes to afford but-3-enyl benzoate (2.3 g, 13.1 mmol, 91% yield) as yellow oil.

Step B: A mixture of but-3-enyl benzoate (500 mg, 2.8 mmol), trimethylsilyl 2,2-difluoro-2-(fluorosulfonyl)acetate (1.4 g, 5.7 mmol) and NaF (5.9 mg, 141.8 μmmol) was heated under neat conditions at 110° C. for 2 hours. After cooling to room temperature, DCM (10 mL) and H₂O (5 mL) were added, separated. The DCM extract was concentrated. The crude residue was purified by silica gel chromatography eluting with 10% EtOAc/hexanes to afford 2-(2,2-difluorocyclopropyl)ethyl benzoate (330 mg, 1.5 mmol, 51% yield) as yellow oil.

Step C: To a suspension of potassium hydroxide (409 mg, 7.3 mmol) in MeOH/H₂O (3:2, 5 mL) was added 2-(2,2-difluorocyclopropyl)ethyl benzoate (330 mg, 1.5 mmol) at 0° C., followed by stirring at room temperature for 1 hour. The reaction was quenched with saturated brine solution (5 mL), and extracted with EtOAc (10 mL×4). The combined organic layer was dried over anhydrous Na₂SO₄ and concentrated under vacuum to afford crude 2-(2,2-difluorocyclopropyl)ethanol (150 mg, 84%) as colorless oil which was used directly in the next step.

Step D:

5-((2-Chlorophenyl)thio)-6′-(2-(2,2-difluorocyclopropyl)ethoxy)-4-hydroxy-2-(thiophen-3-yl)-2,3-dihydro-[2,2′-bipyridin]-6(1H)-one was prepared in 21% yield according to the Example 2, Step A substituting propan-2-ol for 2-(2,2-difluorocyclopropyl)ethanol. ¹H NMR (400 MHz, CD₃OD) δ 7.73 (dd, J=7.6, 7.6 Hz, 1H), 7.43 (d, J=2.8 Hz, 1H), 7.27-7.14 (m, 4H), 7.14 (d, J=2.8 Hz, 1H), 6.93-6.74 (m, 2H), 5.96 (d, J=8.0 Hz, 1H), 4.43 (t, J=3.6 Hz, 2H), 3.88 (d, J=16.0 Hz, 1H), 3.47 (d, J=16.0 Hz, 1H), 1.95-1.67 (m, 3H), 1.36-1.34 (m, 1H), 1.00-0.96 (m, 1H). LCMS M+1=534.9.

### Example 21

### 3-(2-chlorophenyl)sulfanyl-6-[6-[2-(3-methyltriazol-4-yl)phenoxy]-2-pyridyl]-6-(3-thienyl)piperidine-2,4-dione

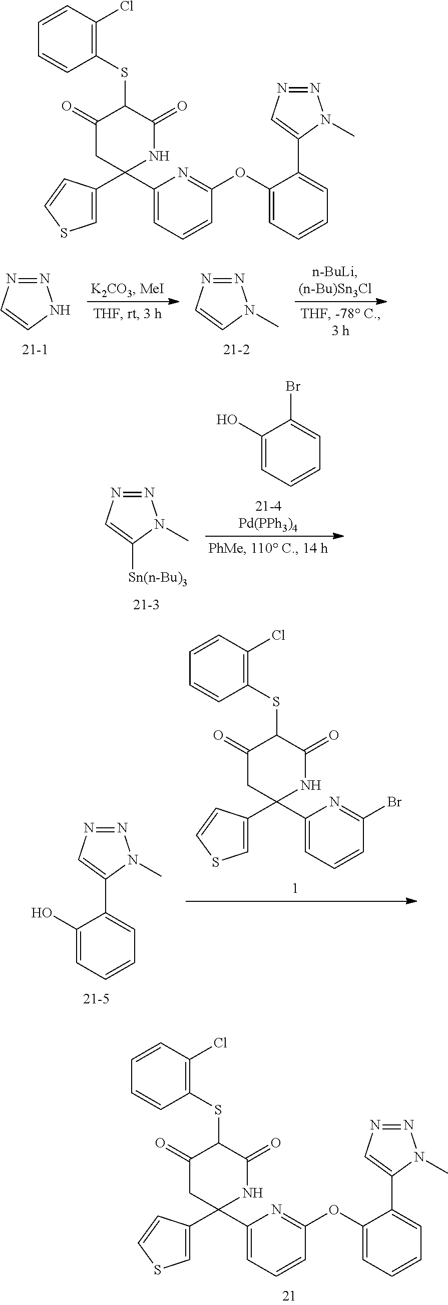

Step A: A solution of 1H-1,2,3-triazole (1.0 g, 14.5 mmol), methyl iodide (3.1 g, 21.7 mmol) and K₂CO₃ (4.0 g, 28.9 mmol) in THF (15 mL) was stirred at room temperature for 3 hours. EtOAc (20 mL) and H₂O (10 mL) were added, separated. The solvent was concentrated under vacuum. The crude residue was purified by silica gel chromatography eluting with 10% MeOH/DCM to afford 1-methyl-1H-1,2,3-triazole (860 mg, 10.4 mmol, 71% yield) as yellow oil.

Step B: To a solution of 1-methyl-1H-1,2,3-triazole (860 mg, 10.4 mmol) in THF (10 mL) at −78° C., was added dropwise n-BuLi (5.0 mL, 12.4 mmol, 2.5 M). The mixture was stirred at −78° C. for 2 hours before addition of Bu₃SnCl (3.7 g, 11.4 mmol). The mixture was stirred at −78° C. for 1 hour and then room temperature for 1 hour. The mixture was concentrated under vacuum and hexane was added. The insoluble material was filtered and the filtrate was concentrated under vacuum to afford 1-methyl-5-(tributylstannyl)-1H-1,2,3-triazole (3.1 g, 80%) as yellow oil which was used directly in the next step.

Step C: A solution of 1-methyl-5-(tributylstannyl)-1H-1,2,3-triazole (3.1 g, 8.3 mmol), 2-bromophenol (1.7 g, 10.0 mmol), Et₃N (1.7 g, 16.7 mmol) and PdCl₂(PPh₃)₂ (1.1 g, 1.7 mmol) in PhMe (16 mL) was stirred at 110° C. for 14 hours. After cooling to room temperature, DCM (25 mL) and H₂O (10 mL) were added, separated. The DCM was concentrated under vacuum. The crude residue was purified by silica gel chromatography eluting with a gradient of 20% EtOAc/hexanes to 10% MeOH/DCM to afford 2-(1-methyl-1H-1,2,3-triazol-5-yl)phenol (110 mg, 8.0% yield) as white solid.

Step D: 5-((2-Chlorophenyl)thio)-4-hydroxy-6′-(2-(1-methyl-1H-1,2,3-triazol-5-yl)phenoxy)-2-(thiophen-3-yl)-2,3-dihydro-[2,2′-bipyridin]-6(1H)-one was prepared in 8.3% yield according to the Example 3, Step A substituting 2-chloro-4-fluoro-phenol for 2-(1-methyl-1H-1,2,3-triazol-5-yl)phenol. ¹H NMR (400 MHz, CD₃OD) δ 7.80 (dd, J=7.6, 7.6 Hz, 1H), 7.47 (d, J=2.8 Hz, 1H), 7.41-7.39 (m, 4H), 7.30 (d, J=2.8 Hz, 1H), 7.21 (d, J=7.6 Hz, 2H), 7.08 (s, 1H), 6.94-6.92 (m, 3H), 6.73 (dd, J=7.2, 7.2 Hz, 1H), 5.94 (d, J=8.0 Hz, 1H), 3.87 (s, 3H), 3.50 (d, J=16.0 Hz, 1H), 3.31 (d, J=16.0 Hz, 1H). LCMS M+1=587.8.

### Example 22

### 3-(2-chlorophenyl)sulfanyl-6-(3-tetrahydropyran-4-yloxyphenyl)-6-(3-thienyl)piperidine-2,4-dione

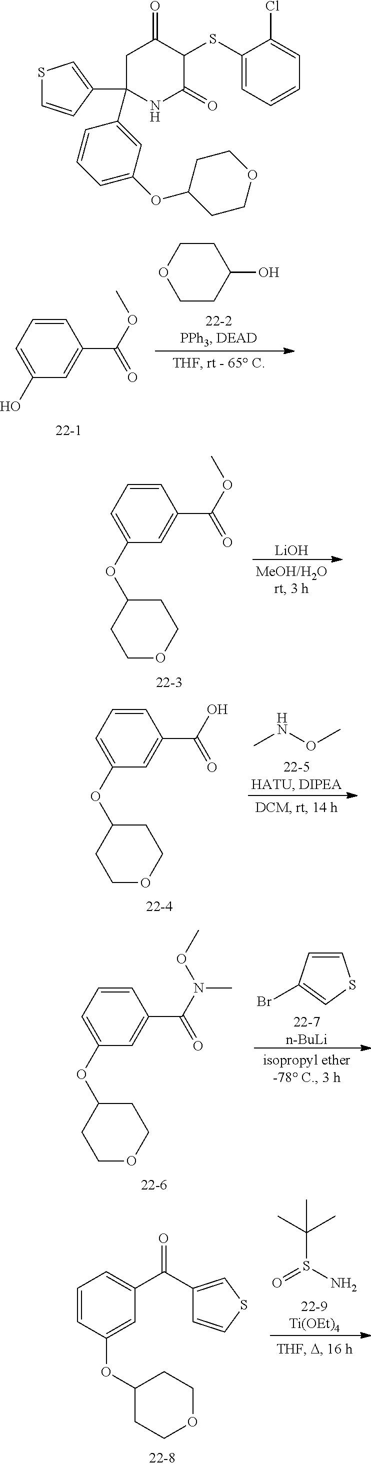

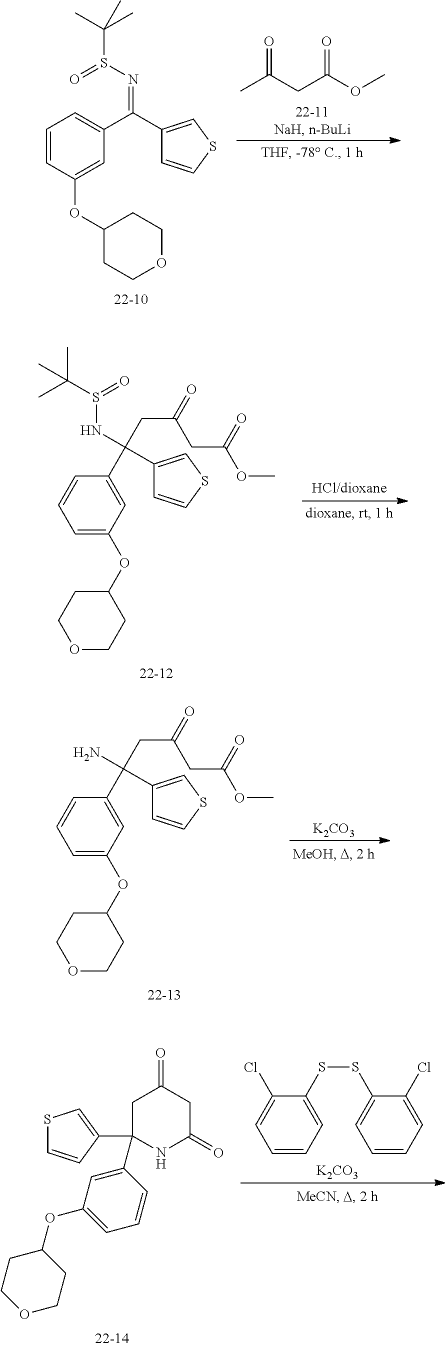

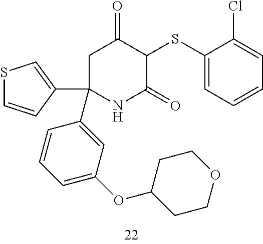 [SMILES: O=C1CC(c2ccsc2)(c2cccc(OC3CCOCC3)c2)NC(=O)C1Sc1ccccc1Cl]

Step A: The suspension of methyl 3-hydroxybenzoate (22.0 g, 144.6 mmol), tetrahydro-2H-pyran-4-ol (22.2 g, 216.9 mmol), PPh₃ (3.8 g, 14.5 mmol) and DEAD (28.0 g, 159.1 mmol) in THF (150 ml) was refluxed for 8 hours. The reaction mixture was then cooled to room temperature, diluted with water (60 ml) and EtOAc (120 mL). The organic layer was separated and concentrated. The crude residue was purified by column chromatography on silica gel with petroleum ether: EtOAc=3:1 as eluent to afford methyl 3-(tetrahydro-2H-pyran-4-yloxy)benzoate (18.1 g, 53% yield) as brown oil.

Step B: A solution of methyl 3-(tetrahydro-2H-pyran-4-yloxy)benzoate (18.1 g, 76.6 mmol) and LiOH (9.2 g, 383 mmol) in methanol/H₂O (80 mL/5 ml) was stirred at room temperature for 3 hours. The reaction mixture was filtered and the filtrate was adjusted to pH=2-3 with aqueous HCl solution (1 M). The resultant solution was extracted with EtOAc (80 mL×2), and concentrated. The crude residue 3-(tetrahydro-2H-pyran-4-yloxy)benzoic acid (14.3 g, 84% yield) as yellow solid was used directly in the next step without further purification.

Step C: N-Methoxy-N-methyl-3-(tetrahydro-2H-pyran-4-yloxy)benzamide was prepared in 85% yield according to the Example 1, Step A substituting 6-bromopicolinic acid for 3-((tetrahydro-2H-pyran-4-yl)oxy)benzoic acid.

Step D: (3-(Tetrahydro-2H-pyran-4-yloxy)phenyl)(thiophen-3-yl)methanone was prepared in 67% yield according to the Example 1, Step B substituting 6-bromo-N-methoxy-N-methylpicolinamide for (3-(tetrahydro-2H-pyran-4-yloxy)phenyl)(thiophen-3-yl)methanone.

Step E: (E)-2-Methyl-N-((3-(tetrahydro-2H-pyran-4-yloxy)phenyl)(thiophen-3-yl)methylene)propane-2-sulfinamide was prepared in 63% yield according to the Example 1, Step C substituting (Z)—N-((6-bromopyridin-2-yl)(thiophen-3-yl)methylene)-2-methylpropane-2-sulfinamide for (E)-2-methyl-N-((3-(tetrahydro-2H-pyran-4-yloxy)phenyl)(thiophen-3-yl)methylene)propane-2-sulfinamide.

Step F: Methyl-5-(1,1-dimethylethylsulfinamido)-3-oxo-5-(3-(tetrahydro-2H-pyran-4-yloxy)phenyl)-5-(thiophen-3-yl)pentanoate was prepared in 58% yield according to the Example 1, Step D substituting methyl 5-(6-bromopyridin-2-yl)-5-(1,1-dimethylethylsulfinamido)-3-oxo-5-(thiophen-3-yl)pentanoate for methyl 5-(1,1-dimethylethylsulfinamido)-3-oxo-5-(3-(tetrahydro-2H-pyran-4-yloxy)phenyl)-5-(thiophen-3-yl)pentanoate.

Step G:

Methyl5-amino-3-oxo-5-(3-(tetrahydro-2H-pyran-4-yloxy)phenyl)-5-(thiophen-3-yl)pentanoate was prepared in 74% yield according to the Example 1, Step E substituting methyl 5-amino-5-(6-bromopyridin-2-yl)-3-oxo-5-(thiophen-3-yl)pentanoate for methyl-5-amino-3-oxo-5-(3-(tetrahydro-2H-pyran-4-yloxy)phenyl)-5-(thiophen-3-yl)pentanoate

Step H:

4-Hydroxy-6-(3-(tetrahydro-2H-pyran-4-yloxy)phenyl)-6-(thiophen-3-yl)-5,6-dihydropyridin-2(1H)-one was prepared in 87% yield according to the Example 1, Step F substituting 6′-bromo-4-hydroxy-2-(thiophen-3-yl)-2,3-dihydro-[2,2′-bipyridin]-6(1H)-one for 4-hydroxy-6-(3-(tetrahydro-2H-pyran-4-yloxy)phenyl)-6-(thiophen-3-yl)-5,6-dihydropyridin-2(1H)-one

Step I:

3-(2-Chlorophenylthio)-4-hydroxy-6-(3-(tetrahydro-2H-pyran-4-yloxy)phenyl)-6-(thiophen-3-yl)-5,6-dihydropyridin-2(1H)-one was prepared in 5.0% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for 3-(2-chlorophenylthio)-4-hydroxy-6-(3-(tetrahydro-2H-pyran-4-yloxy)phenyl)-6-(thiophen-3-yl)-5,6-dihydropyridin-2(1H)-one. ¹H NMR (400 MHz, CD₃OD) δ 7.48 (dd, J=8.0, 8.0 Hz, 1H), 7.32 (dd, J=2.0, 2.0 Hz, 1H), 7.22 (d, J=8.0 Hz, 1H), 7.21 (d, J=8.0 Hz, 1H), 7.18 (d, J=8.0 Hz, 1H), 7.16-7.14 (m, 2H), 7.15-7.03 (m, 2H), 6.69 (d, J=3.2 Hz, 1H), 5.92 (dd, J=7.6, 2.4 Hz, 1H), 4.54-4.50 (m, 1H), 3.89 (t, J=5.6 Hz, 2H), 3.56 (d, J=16.0 Hz, 1H), 3.54 (t, J=5.6 Hz, 2H), 3.51 (d, J=16.0 Hz, 1H), 1.98-1.92 (m, 2H), 1.70-1.60 (m, 2H). LCMS M+1=513.9.

### Example 23

### 3-(2-chlorophenyl)sulfanyl-1-methyl-6-(3-tetrahydropyran-4-yloxyphenyl)-6-(3-thienyl)piperidine-2,4-dione

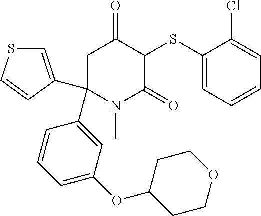 [SMILES: CN1C(=O)C(Sc2ccccc2Cl)C(=O)CC1(c1ccsc1)c1cccc(OC2CCOCC2)c1]

Step A: To a suspension of NaH (60% weight, 47 mg, 1.2 mmol) in anhydrous THF (5 mL) was added dropwise methyl iodide (166 mg, 1.2 mmol) at 0° C. under nitrogen atmosphere and then the reaction was stirred for 30 minutes. The compound of example 22 (200 mg, 389 μmol) in THF (3 mL) was added dropwise to the reaction mixture and the reaction was stirred at 0° C. for 1 hour followed by stirring at room temperature for another 1 hour. The reaction was quenched by HCl solution (1 M), and separated. The solvent was removed. The crude residue was purified by preparative HPLC (formic acid) to give the desired product (5.5 mg, 3% yield) as white solid. ¹H NMR (400 MHz, CD₃OD) δ 7.58 (dd, J=8.0, 8.0 Hz, 1H), 7.57 (dd, J=2.0, 2.0 Hz, 1H), 7.37 (d, J=8.0 Hz, 1H), 7.23 (d, J=8.0 Hz, 1H), 7.21 (d, J=8.0 Hz, 1H), 7.16 (d, J=7.2 Hz, 1H), 7.15-7.02 (m, 2H), 6.97-6.79 (m, 2H), 6.10 (dd, J=8.0, 1.2 Hz, 1H), 4.51-4.49 (m, 1H), 3.90 (t, J=5.6 Hz, 2H), 3.66 (d, J=16.0 Hz, 1H), 3.55 (t, J=5.6 Hz, 2H), 3.51 (d, J=16.0 Hz, 1H), 2.84 (s, 3H), 1.96-1.92 (m, 2H), 1.69-1.63 (m, 2H). LCMS M+1=527.9.

### Example 24

### 3-(2-chlorophenyl)sulfanyl-6-[3-(4-fluorophenoxy)phenyl]-6-(3-thienyl)piperidine-2,4-dione

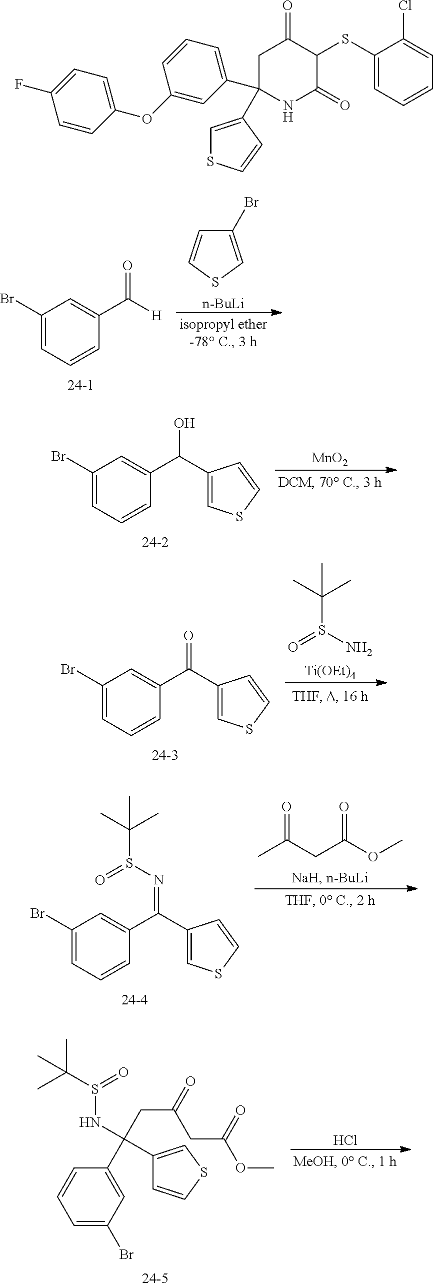

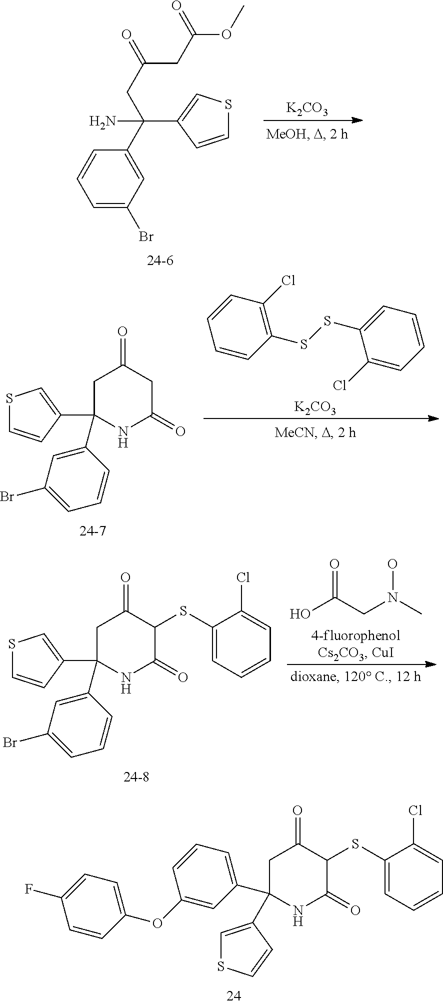

Step A:

(3-Bromophenyl)(thiophen-3-yl)methanol was prepared in 95% yield according to the Example 7, Step A substituting 4-bromobenzaldehyde for 3-bromobenzaldehyde.

Step B:

(3-Bromophenyl)(thiophen-3-yl)methanone was prepared in 95% yield according to the Example 7 Step B substituting (4-bromophenyl)(thiophen-3-yl)methanol for (3-bromophenyl)(thiophen-3-yl)methanol.

Step C:

(E)-N-((3-Bromophenyl)(thiophen-3-yl)methylene)-2-methylpropane-2-sulfinamide was prepared in 97% yield according to the Example 1, Step C substituting (6-bromopyridin-2-yl)(thiophen-3-yl)methanone for (3-bromophenyl)(thiophen-3-yl)methanone.

Step D:

Methyl 5-(3-bromophenyl)-5-(1,1-dimethylethylsulfinamido)-3-oxo-5-(thiophen-3-yl)pentanoate was prepared in 68% yield according to the Example 1, Step D substituting (Z)—N-((6-bromopyridin-2-yl)(thiophen-3-yl)methylene)-2-methylpropane-2-sulfinamide for (E)-N-((3-bromophenyl) (thiophen-3-yl)methylene)-2-methylpropane-2-sulfinamide.

Step E:

Methyl 5-amino-5-(3-bromophenyl)-3-oxo-5-(thiophen-3-yl)pentanoate was prepared in 74% yield according to the Example 1, Step E substituting methyl 5-amino-5-(6-bromopyridin-2-yl)-3-oxo-5-(thiophen-3-yl)pentanoate for methyl 5-(3-bromophenyl)-5-(1,1-dimethylethylsulfinamido)-3-oxo-5-(thiophen-3-yl)pentanoate.

Step F:

6-(4-Bromophenyl)-4-hydroxy-6-(thiophen-3-yl)-5,6-dihydropyridin-2(1H)-one was prepared in 80% yield according to the Example 1, Step F substituting 6′-bromo-4-hydroxy-2-(thiophen-3-yl)-2,3-dihydro-[2,2′-bipyridin]-6(1H)-one for methyl 5-amino-5-(3-bromophenyl)-3-oxo-5-(thiophen-3-yl)pentanoate.

Step G:

6-(3-Bromophenyl)-3-((2-chlorophenyl)thio)-4-hydroxy-6-(thiophen-3-yl)-5,6-dihydropyridin-2(1H)-one was prepared in 92% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for 6-(3-bromophenyl)-4-hydroxy-6-(thiophen-3-yl)-5,6-dihydropyridin-2(1H)-one.

Step H:

3-((2-Chlorophenyl)thio)-6-(3-(4-fluorophenoxy)phenyl)-6-(thiophen-3-yl)piperidine-2,4-dione was prepared in 6.6% yield according to the Example 3, Step A substituting chloro-4-fluoro-phenol for 4-fluorophenol and 6′-bromo-5-((2-chlorophenyl)thio)-4-hydroxy-2-(thiophen-3-yl)-2,3-dihydro-[2,2′-bipyridin]-6(1H)-one for 6-(3-bromophenyl)-3-((2-chlorophenyl)thio)-4-hydroxy-6-(thiophen-3-yl)-5,6-dihydropyridin-2(111H)-on e. ¹H NMR (400 MHz, CD₃OD) δ 7.49 (dd, J=4.8, 2.8 Hz, 1H), 7.29 (dd, J=8.0, 8.0 Hz, 1H), 7.30-7.26 (m, 1H), 7.23-7.21 (m, 2H), 7.14 (d, J=4.8 Hz, 1H), 7.06-7.02 (m, 3H), 6.96-6.92 (m, 4H), 6.80-6.75 (m, 1H), 5.98 (dd, J=7.6, 1.2 Hz, 1H), 3.47-3.45 (m, 2H). LCMS M+1=523.8.

### Example 25

### 6-(6-bromo-5-morpholino-2-pyridyl)-3-(2-chlorophenyl)sulfanyl-6-(3-thienyl)piperidine-2,4-dione

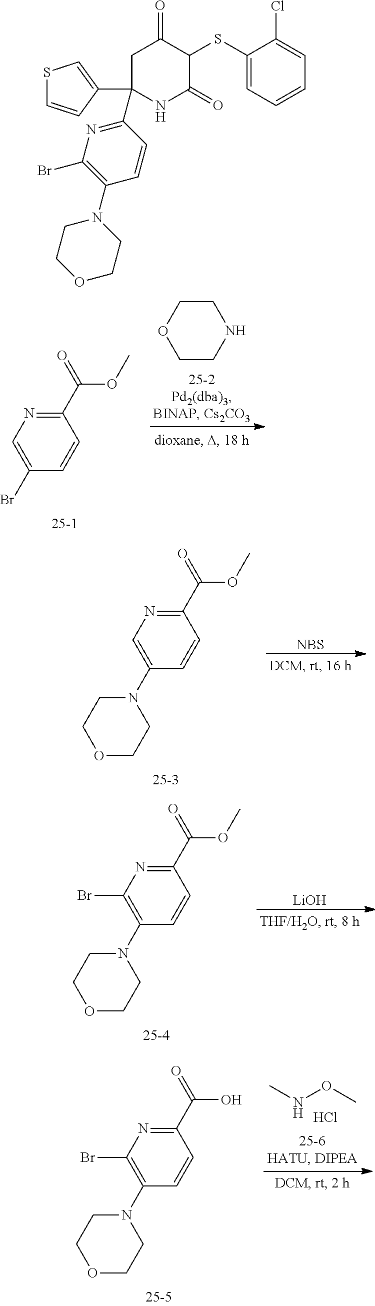

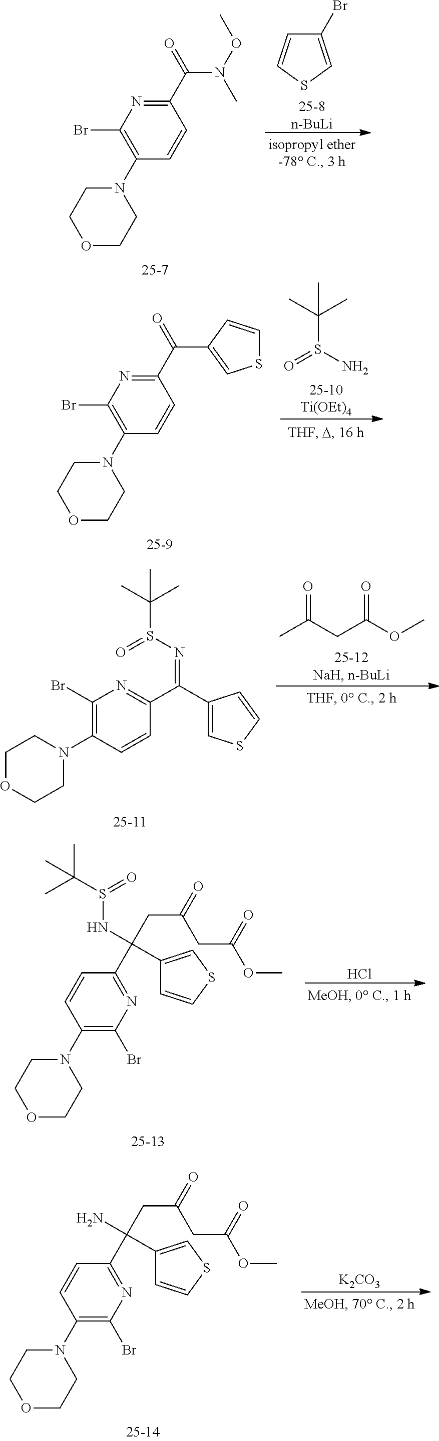

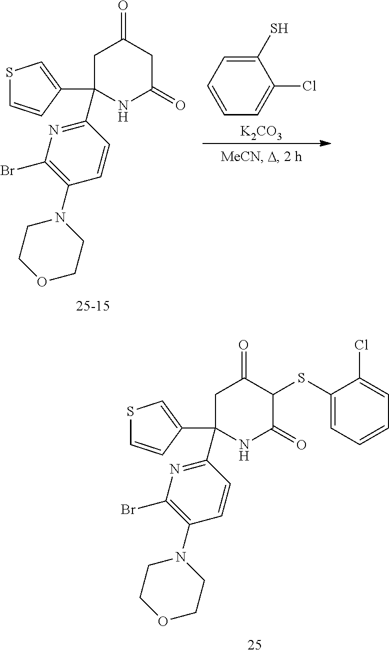

Step A: Methyl 5-bromopicolinate (60.0 g, 277 mmol), morpholine (72 g, 833 mmol), Pd₂(dba)₃ (5.0 g, 5.55 mmol), 2,2′-bis(diphenylphosphino)-1,1′-binaphthyl (6.9 g, 11.1 mmol) and Cs₂CO₃ (135 g, 417 mmol) were combined in a flask (2 L). Dioxane (1 L) was added, and the mixture was stirred at 120° C. for 18 h under nitrogen atmosphere. The reaction mixture was cooled to room temperature, filtered and washed with EtOAc (300 ml×3). The filtrate was dried over anhydrous MgSO₄ and concentrated. Silica gel chromatography eluting with 50% EtOAc/hexanes provided methyl 5-morpholinopicolinate (25 g, 112.6 mmol, 40% yield) as yellow solid.

Step B: Methyl 5-morpholinopicolinate (25.0 g, 113 mmol) in DCM (500 ml), N-bromosuccinimide (22 g, 123 mmol) was added. The mixture was stirred at room temperature for 16 hours. The reaction mixture was concentrated. The residue was purified by silica gel chromatography to afford methyl 6-bromo-5-morpholinopicolinate (23 g, 69.6 mmol, 62% yield) as yellow solid.

Step C: 6-Bromo-5-morpholinopicolinate (23.0 g, 69.6 mmol) in THF (200 ml), LiOH (9.62 g, 229.1 mmol) in H₂O (100 ml) was added. The reaction mixture was stirred at room temperature for 8 hours. The mixture was concentrated, the resultant aqueous solution was adjusted to pH<4 with HCl solution (1 M), extracted with DCM (100 ml×3), dried with anhydrous Na₂SO₄, and concentrated to afford 6-bromo-5-morpholinopicolinic acid (21.0 g, 73.1 mmol, 96%) as yellow solid.

Step D: 6-Bromo-N-methoxy-N-methyl-5-morpholinopicolinamide was prepared in 75% yield according to the Example 1, Step A substituting 6-bromopicolinic acid for 6-bromo-5-morpholinopicolinic acid.

Step E: (6-Bromo-5-morpholinopyridin-2-yl)(thiophen-3-yl)methanone was prepared in 22% yield according to the Example 1, Step B substituting 6-bromo-N-methoxy-N-methylpicolinamide for 6-bromo-N-methoxy-N-methyl-5-morpholinopicolinamide.

Step F: (Z)—N-((6-Bromo-5-morpholinopyridin-2-yl)(thiophen-3-yl)methylene)-2-methylpropane-2-sulfinamide was prepared in 82% yield according to the Example 1, Step C substituting (6-bromopyridin-2-yl)(thiophen-3-yl)methanone for (6-bromo-5-morpholinopyridin-2-yl)(thiophen-3-yl)methanone.

Step G:

Methyl 5-(6-bromo-5-morpholinopyridin-2-yl)-5-(1,1-dimethylethylsulfinamido)-3-oxo-5-(thiophen-3-yl)pentanoate was prepared in 84% yield according to the Example 1, Step D substituting (Z)—N-((6-bromopyridin-2-yl)(thiophen-3-yl)methylene)-2-methylpropane-2-sulfinamide for (Z)—N-((6-bromo-5-morpholinopyridin-2-yl)-(thiophen-3-yl)methylene)-2-methylpropane-2-sulfinamide.

Step H:

Methyl 5-amino-5-(6-bromo-5-morpholinopyridin-2-yl)-3-oxo-5-(thiophen-3-yl)pentanoate was prepared in 88% yield according to the Example 1, Step E substituting methyl 5-amino-5-(6-bromopyridin-2-yl)-3-oxo-5-(thiophen-3-yl)pentanoate for Methyl 5-(6-bromo-5-morpholinopyridin-2-yl)-5-(1,1-dimethylethylsulfinamido)-3-oxo-5-(thiophen-3-yl)pentanoate.

Step I: 6′-Bromo-4-hydroxy-5′-morpholino-2-(thiophen-3-yl)-2,3-dihydro-[2,2′-bipyridin]-6(1H)-one was prepared in 89% yield according to the Example 1, Step F substituting 6′-bromo-4-hydroxy-2-(thiophen-3-yl)-2,3-dihydro-[2,2′-bipyridin]-6(1H)-one for Methyl 5-(6-bromo-5-morpholinopyridin-2-yl)-5-(1,1-dimethylethylsulfinamido)-3-oxo-5-(thiophen-3-yl)pentanoate.

Step J: 6′-Bromo-5-((2-chlorophenyl)thio)-4-hydroxy-5′-morpholino-2-(thiophen-3-yl)-2,3-dihydro-[2,2′-bipyridin]-6(1H)-one was prepared in 36% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for Methyl 5-amino 5-(6-bromo-5-morpholinopyridin-2-yl)-3-oxo-5-(thiophen-3-yl) pentanoate. ¹H NMR (400 MHz, (CD₃)₂SO) δ 11.70 (br s, 1H), 8.47 (s, 1H), 7.65 (d, J=8.0 Hz, 1H), 7.63 (d, J=8.0 Hz, 1H), 7.53-7.51 (m, 1H), 7.31-7.27 (m, 2H), 7.12 (dd, J=5.2, 1.6 Hz, 1H), 6.96 (d, J=8.0 Hz, 1H), 6.81-6.78 (m, 1H), 5.95 (dd, J=8.0, 1.2 Hz, 1H), 3.80-3.72 (m, 5H), 3.36 (d, J=16.4 Hz, 1H), 3.01-2.99 (m, 4H). LCMS M+1=579.8.

### Example 26

### 3-(2-chlorophenyl)sulfanyl-6-[6-(4-fluoroanilino)-5-morpholino-2-pyridyl]-6-(3-thienyl)piperidine-2,4-dione

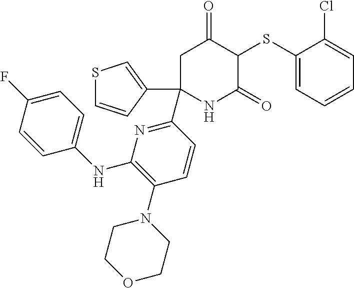 [SMILES: O=C1CC(c2ccsc2)(c2ccc(N3CCOCC3)c(Nc3ccc(F)cc3)n2)NC(=O)C1Sc1ccccc1Cl]

Step A: 5-((2-Chlorophenyl)thio)-6′-((4-fluorophenyl)amino)-4-hydroxy-5′-morpholino-2-(thiophen-3-yl)-2,3-dihydro-[2,2′-bipyridin]-6(1H)-one was prepared in 6% yield according to the Example 4, step A substituting cyclohexanamine for 4-fluoroaniline and 6′-bromo-5-((2-chlorophenyl)thio)-4-hydroxy-2-(thiophen-3-yl)-2,3-dihydro-[2,2′-bipyridin]-6(1H)-one 1 for 6′-bromo-5-((2-chlorophenyl)thio)-4-hydroxy-5′-morpholino-2-(thiophen-3-yl)-2,3-dihydro-[2,2′-bipyridin]-6(1H)-one. ¹H NMR (400 MHz, CD₃OD) δ 7.61-7.58 (m, 2H), 7.50 (d, J=8.0 Hz, 1H), 7.49-7.48 (m, 1H), 7.33 (s, 1H), 7.28-7.22 (m, 1H), 7.17-7.12 (m, 1H), 7.06-7.01 (m, 4H), 6.86-6.82 (m, 1H), 6.25 (d, J=8.0 Hz, 1H), 3.93 (m, 4H), 3.82 (d, J=16.4 Hz, 1H), 3.49 (d, J=16.4 Hz, 1H), 2.98-2.96 (m, 4H). LCMS M+1=608.8.

### Example 27

### 3-(2-chlorophenyl)sulfanyl-6-[6-(4-fluorophenoxy)-5-morpholino-2-pyridyl]-6-(3-thienyl)piperidine-2,4-dione

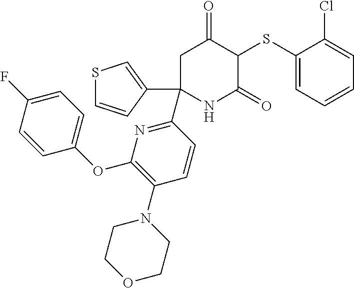 [SMILES: O=C1CC(c2ccsc2)(c2ccc(N3CCOCC3)c(Oc3ccc(F)cc3)n2)NC(=O)C1Sc1ccccc1Cl]

5-((2-Chlorophenyl)thio)-6′-(4-fluorophenoxy)-4-hydroxy-5′-morpholino-2-(thiophen-3-yl)-2,3-dihydro-[2,2′-bipyridin]-6(1H)-one was prepared in 3% yield according to the Example 2, Step A substituting propan-2-ol for 4-fluorophenol and 6′-bromo-5-((2-chlorophenyl)thio)-4-hydroxy-2-(thiophen-3-yl)-2,3-dihydro-[2,2′-bipyridin]-6(1H)-one 1 for 6′-bromo-5-((2-chlorophenyl)thio)-4-hydroxy-5′-morpholino-2-(thiophen-3-yl)-2,3-dihydro-[2,2′-bipyridin]-6(1H)-one. ¹H NMR (400 MHz, CD₃OD) δ 7.42 (d, J=8.4 Hz, 1H), 7.37-7.36 (m, 1H), 7.26-7.22 (m, 2H), 7.12-7.01 (m, 5H), 6.98-6.94 (m, 2H), 6.82-6.78 (m, 1H), 6.02 (dd, J=8.0, 1.2 Hz, 1H), 3.87-3.84 (m, 4H), 3.52 (d, J=16.8 Hz, 1H), 3.24-3.17 (m, 5H). LCMS M+1=609.8.

### Example 28

### 3-(2-chlorophenyl)sulfanyl-6-[4-(3-hydroxypropoxy)phenyl]-6-(3-thienyl)piperidine-2,4-dione

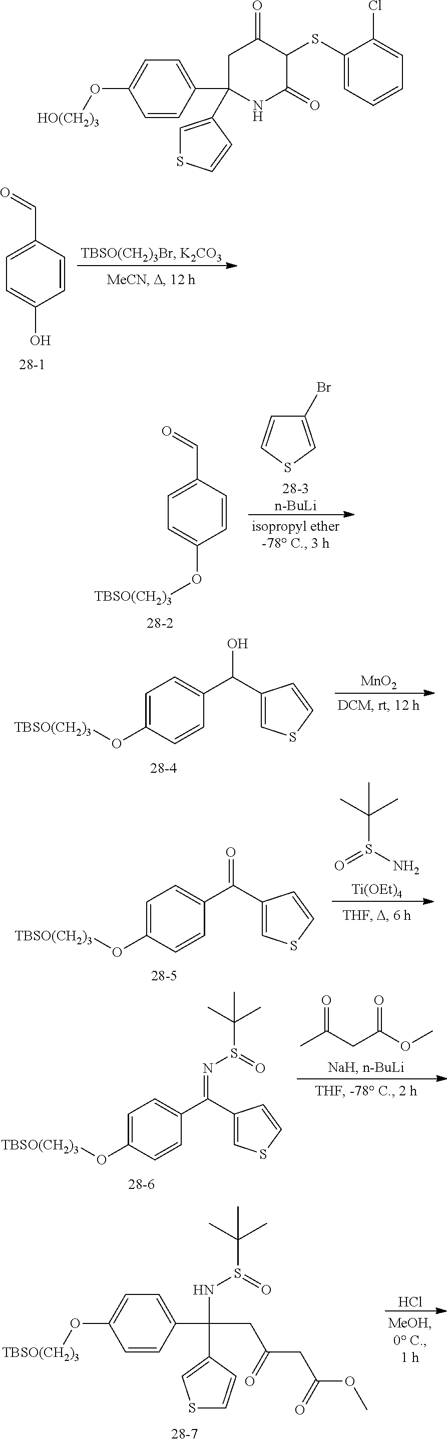

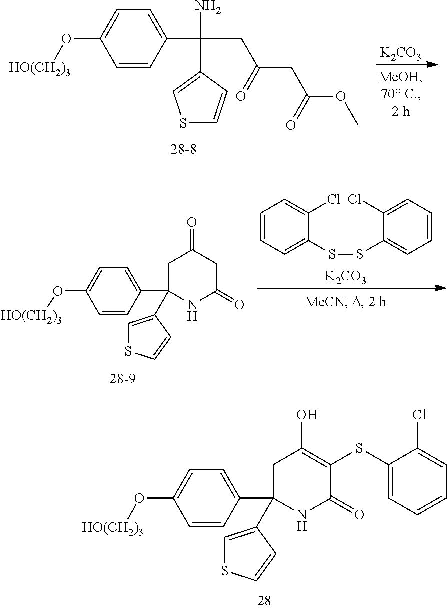

Step A: To a solution of 4-hydroxybenzaldehyde (25 g, 205 mmol) and (3-bromopropoxy)(tert-butyl)dimethylsilane (57 g, 225 mmol) in MeCN (200 mL) was added K₂CO₃ (85 g, 614 mmol). The reaction mixture was heated at 80° C. for 12 hours. After cooling to room temperature, DCM (50 mL) was added, and the mixture was filtered over Celite. Then the filtrate was concentrated, purified by silica gel column (petroleum ether/EtOAc=20/1) to afford 4-(3-((tert-butyldimethylsilyl)oxy)propoxy)benzaldehyde (36 g, 60% yield) as white solid.

Step B: (4-(3-((tert-Butyldimethylsilyl)oxy)propoxy)phenyl)(thiophen-3-yl)methanol was prepared in 84% yield according to the Example 7, Step A substituting 4-bromobenzaldehyde for 4-(3-((tert-butyldimethylsilyl)oxy)propoxy)benzaldehyde.

Step C: (4-(3-((tert-Butyldimethylsilyl)oxy)propoxy)phenyl)(thiophen-3-yl)methanone was prepared in 56% yield according to the Example 7, Step B substituting (4-bromophenyl)(thiophen-3-yl)methanone for (4-(3-((tert-butyldimethylsilyl)oxy)propoxy)phenyl)(thiophen-3-yl)methanol.

Step D: (Z)—N-((4-(3-((tert-butyldimethylsilyl)oxy)propoxy)phenyl)(thiophen-3-yl)methylene)-2-methylpropane-2-sulfinamide was prepared in 71% yield according to the Example 7, Step C substituting (E)-N-((4-bromophenyl) (thiophen-3-yl)methylene)-2-methylpropane-2-sulfinamide for (4-(3-((tert-butyldimethylsilyl)oxy)propoxy)phenyl)(thiophen-3-yl)methanone.

Step E: Methyl 5-(4-(3-((tert-butyldimethylsilyl)oxy)propoxy)phenyl)-5-(1,1-dimethylethylsulfinamido)-3-oxo-5-(thio phen-3-yl)pentanoate was prepared in 82% yield according to the Example 7, Step D substituting (E)-N-((4-bromophenyl) (thiophen-3-yl)methylene)-2-methylpropane-2-sulfinamide for (Z)—N-((4-(3-((tert-butyldimethylsilyl)oxy)propoxy)phenyl)(thiophen-3-yl)methylene)-2-methylpropane-2-sulfinamide.

Step F: Methyl 5-amino-5-(4-(3-hydroxypropoxy)phenyl)-3-oxo-5-(thiophen-3-yl)pentanoate was prepared in 82% yield according to the Example 7, Step E substituting methyl 5-(4-bromophenyl)-5-(1,1-dimethylethylsulfinamido)-3-oxo-5-(thiophen-3-yl)pentanoate for methyl 5-(4-(3-((tert-butyldimethylsilyl)oxy)propoxy)phenyl)-5-(1,1-dimethylethylsulfinamido)-3-oxo-5-(thio phen-3-yl)pentanoate.

Step G: 6-(4-(3-Hydroxypropoxy)phenyl)-6-(thiophen-3-yl)piperidine-2,4-dione was prepared in 90% yield according to the Example 7, Step F substituting methyl 5-amino-5-(4-bromophenyl)-3-oxo-5-(thiophen-3-yl)pentanoate for methyl 5-amino-5-(4-(3-hydroxypropoxy)phenyl)-3-oxo-5-(thiophen-3-yl)pentanoate.

Step H: 3-((2-Chlorophenyl)thio)-4-hydroxy-6-(4-(3-hydroxypropoxy)phenyl)-6-(thiophen-3-yl)-5,6-dihydropyridin-2(1H)-one was prepared in 29% yield according to the Example 7, Step G substituting 6-(4-bromophenyl)-4-hydroxy-6-(thiophen-3-yl)-5,6-dihydropyridin-2(1H)-one for 6-(4-(3-hydroxypropoxy)phenyl)-6-(thiophen-3-yl)piperidine-2,4-dione. ¹H NMR (400 MHz, (CD₃)₂SO) δ 11.41 (s, 1H), 8.41 (s, 1H), 7.55 (dd, J=5.0, 3.0 Hz, 1H), 7.25-7.30 (m, 4H), 7.12 (d, J=5.1 Hz, 1H), 6.88-6.96 (m, 3H), 6.72 (dd, J=7.6, 7.6 Hz, 1H), 5.84 (dd, J=6.0, 1.2 Hz, 1H), 4.45 (s, 1H), 4.00 (t, J=6.3 Hz, 2H), 3.52 (t, J=6.2 Hz, 2H), 3.39-3.46 (m, 2H), 1.85-1.79 (m, 2H). LCMS M+1=487.9.

### Example 29

### 3-(2-chlorophenyl)sulfanyl-6-[4-(2-hydroxyethoxy)phenyl]-6-(3-thienyl)piperidine-2,4-dione

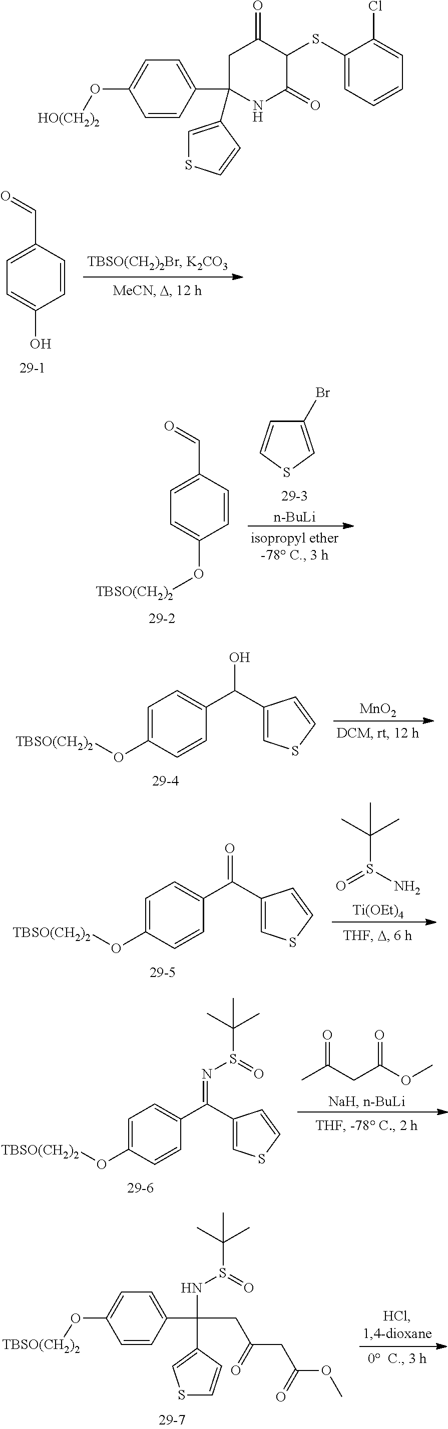

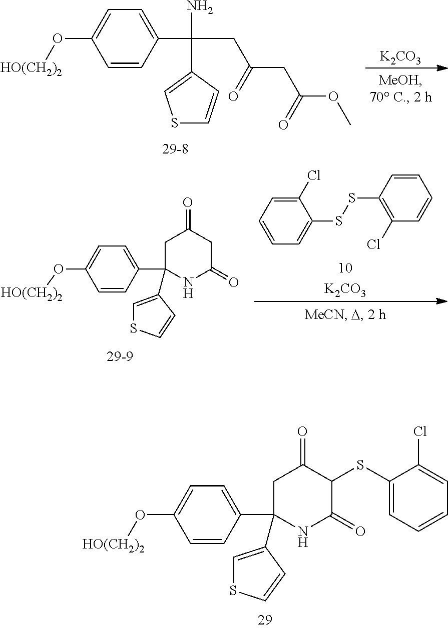

Step A: 4-(2-((tert-Butyldimethylsilyl)oxy)ethoxy)benzaldehyde was prepared in 90% yield according to the Example 27, Step A substituting (3-bromopropoxy)(tert-butyl)dimethylsilane for (2-bromoethoxy)(tert-butyl)dimethylsilane.

Step B: (4-(2-((tert-Butyldimethylsilyl)oxy)ethoxy)phenyl)(thiophen-3-yl)methanol was prepared in 26% yield according to the Example 7, Step A substituting 4-bromobenzaldehyde for 4-(3-((tert-butyldimethylsilyl)oxy)propoxy)benzaldehyde.

Step C: (4-(2-((tert-Butyldimethylsilyl)oxy)ethoxy)phenyl)(thiophen-3-yl)methanone was prepared in 97% yield according to the Example 7, Step B substituting (4-bromophenyl)(thiophen-3-yl)methanone for (4-(2-((tert-butyldimethylsilyl)oxy)ethoxy)phenyl)(thiophen-3-yl)methanol.

Step D: (Z)—N-((4-(2-((tert-butyldimethylsilyl)oxy)ethoxy)phenyl)(thiophen-3-yl)methylene)-2-methylpropane-2-sulfinamide was prepared in 48% yield according to the Example 7, Step C substituting (E)-N-((4-bromophenyl) (thiophen-3-yl)methylene)-2-methylpropane-2-sulfinamide for (4-(2-((tert-butyldimethylsilyl)oxy)ethoxy)phenyl)(thiophen-3-yl)methanone.

Step E: Methyl 5-(4-(2-((tert-butyldimethylsilyl)oxy)ethoxy)phenyl)-5-(1,1-dimethylethylsulfinamido)-3-oxo-5-(thiophen-3-yl)pentanoate was prepared in 66% yield according to the Example 7, Step D substituting (E)-N-((4-bromophenyl) (thiophen-3-yl)methylene)-2-methylpropane-2-sulfinamide for (Z)—N-((4-(2-((tert-butyldimethylsilyl)oxy)ethoxy)phenyl)(thiophen-3-yl)methylene)-2-methylpropane-2-sulfinamide.

Step F: Methyl 5-amino-5-(4-(2-hydroxyethoxy)phenyl)-3-oxo-5-(thiophen-3-yl)pentanoate was prepared in 79% yield according to the Example 7, Step E substituting methyl 5-(4-bromophenyl)-5-(1,1-dimethylethylsulfinamido)-3-oxo-5-(thiophen-3-yl)pentanoate for methyl 5-(4-(2-((tert-butyldimethylsilyl)oxy)ethoxy)phenyl)-5-(1,1-dimethylethylsulfinamido)-3-oxo-5-(thiophen-3-yl)pentanoate.

Step G: 6-(4-(2-Hydroxyethoxy)phenyl)-6-(thiophen-3-yl)piperidine-2,4-dione was prepared in 93% yield according to the Example 7, Step F substituting methyl 5-amino-5-(4-bromophenyl)-3-oxo-5-(thiophen-3-yl)pentanoate for methyl 5-amino-5-(4-(2-hydroxyethoxy)phenyl)-3-oxo-5-(thiophen-3-yl)pentanoate.

Step H: 3-((2-Chlorophenyl)thio)-6-(4-(2-hydroxyethoxy)phenyl)-6-(thiophen-3-yl)piperidine-2,4-dione was prepared in 35% yield according to the Example 7, Step G substituting 6-(4-bromophenyl)-4-hydroxy-6-(thiophen-3-yl)-5,6-dihydropyridin-2(1H)-one for 6-(4-(2-hydroxyethoxy)phenyl)-6-(thiophen-3-yl)piperidine-2,4-dione. ¹H NMR (400 MHz, CD₃OD) δ 8.38 (s, 1H), 7.54 (dd, J=4.8, 2.8 Hz, 1H), 7.29-7.25 (m, 4H), 7.13 (dd, J=5.2, 1.2 Hz, 1H), 6.95-6.89 (m, 3H), 6.74-6.69 (m, 1H), 5.85 (dd, J=8.0, 1.2 Hz, 1H), 4.84 (s, 1H), 3.96 (t, J=4.8 Hz, 2H), 3.66 (d, J=4.4 Hz, 2H), 3.34 (d, J=4.4 Hz, 2H). LCMS M+1=473.8.

### Example 30

### 3-(2-chlorophenyl)sulfanyl-6-[4-(2-methoxyethoxy)phenyl]-6-(3-thienyl)piperidine-2,4-dione

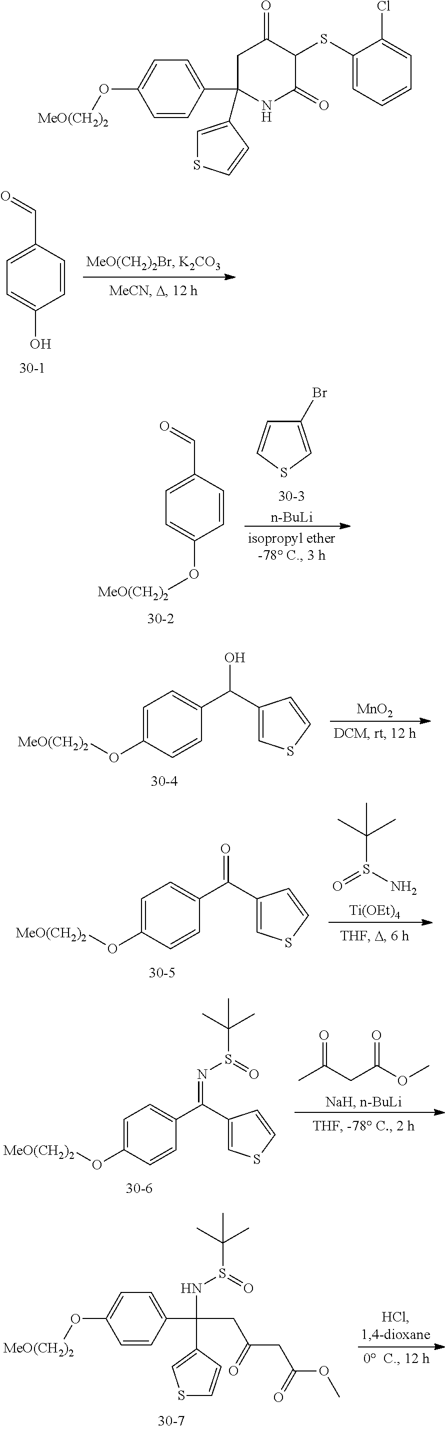

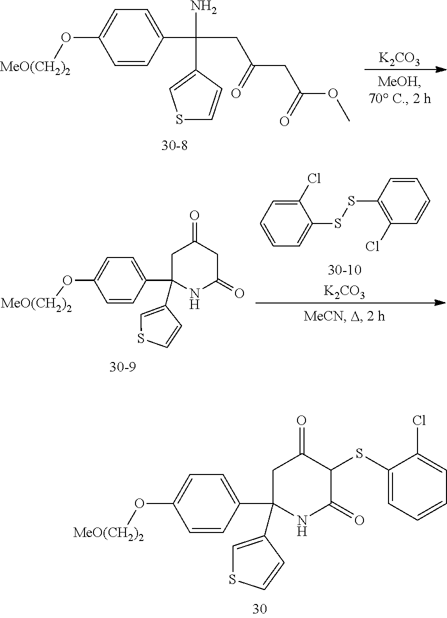

Step A: 4-(2-Methoxyethoxy)benzaldehyde was prepared in 81% yield according to the Example 27, Step A substituting (3-bromopropoxy)(tert-butyl)dimethylsilane for 1-bromo-2-methoxyethane.

Step B: (4-(2-Methoxyethoxy)phenyl)(thiophen-3-yl)methanol was prepared in 91% yield according to the Example 7, Step A substituting 4-bromobenzaldehyde for 4-(2-methoxyethoxy)benzaldehyde.

Step C: (4-(2-Methoxyethoxy)phenyl)(thiophen-3-yl)methanone was prepared in 50% yield according to the Example 7, Step B substituting (4-bromophenyl)(thiophen-3-yl)methanone for (4-(2-methoxyethoxy)phenyl) (thiophen-3-yl)methanol.

Step D: (Z)—N-((4-(2-Methoxyethoxy)phenyl)(thiophen-3-yl)methylene)-2-methylpropane-2-sulfinamide was prepared in 58% yield according to the Example 7, Step C substituting (E)-N-((4-bromophenyl) (thiophen-3-yl)methylene)-2-methylpropane-2-sulfinamide for (4-(2-methoxyethoxy)phenyl)(thiophen-3-yl)methanone.

Step E: Methyl 5-(1,1-dimethylethylsulfinamido)-5-(4-(2-methoxyethoxy)phenyl)-3-oxo-5-(thiophen-3-yl)pentanoate was prepared in 78% yield according to the Example 7, Step D substituting (E)-N-((4-bromophenyl) (thiophen-3-yl)methylene)-2-methylpropane-2-sulfinamide for (Z)—N-((4-(2-methoxyethoxy)phenyl)(thiophen-3-yl)methylene)-2-methylpropane-2-sulfinamide.

Step F: Methyl 5-amino-5-(4-(2-methoxyethoxy)phenyl)-3-oxo-5-(thiophen-3-yl)pentanoate in 90% yield according to the Example 7, Step E substituting methyl 5-(4-bromophenyl)-5-(1,1-dimethylethylsulfinamido)-3-oxo-5-(thiophen-3-yl)pentanoate for methyl 5-(1,1-dimethylethylsulfinamido)-5-(4-(2-methoxyethoxy)phenyl)-3-oxo-5-(thiophen-3-yl)pentanoate.

Step G: 6-(4-(2-Methoxyethoxy)phenyl)-6-(thiophen-3-yl)piperidine-2,4-dione was prepared in 26% yield according to the Example 7, Step F substituting methyl 5-amino-5-(4-bromophenyl)-3-oxo-5-(thiophen-3-yl)pentanoate for methyl 5-amino-5-(4-(2-methoxyethoxy)phenyl)-3-oxo-5-(thiophen-3-yl)pentanoate.

Step H: 3-((2-Chlorophenyl)thio)-6-(4-(2-methoxyethoxy)phenyl)-6-(thiophen-3-yl)piperidine-2,4-dione was prepared in 30% yield according to the Example 7, Step G substituting 6-(4-bromophenyl)-4-hydroxy-6-(thiophen-3-yl)-5,6-dihydropyridin-2(1H)-one for 6-(4-(2-methoxyethoxy)phenyl)-6-(thiophen-3-yl)piperidine-2,4-dione. ¹H NMR (400 MHz, (CD₃)₂SO) δ 8.45 (s, 1H), 7.58 (dd, J=5.0, 3.0 Hz, 1H), 7.28-7.33 (m, 4H), 7.16 (dd, J=5.1, 1.1 Hz, 1H), 6.93-6.98 (m, 3H), 6.72-6.76 (m, 1H), 5.87 (dd, J=8.0, 1.2 Hz, 1H), 4.09-4.11 (m, 2H), 3.65-3.67 (m, 2H), 3.42 (s, 2H), 3.31 (s, 3H). LCMS M+1=487.9.

### Example 31

### 3-(2-chlorophenyl)sulfanyl-6-[4-(3-methoxypropoxy)phenyl]-6-(3-thienyl)piperidine-2,4-dione

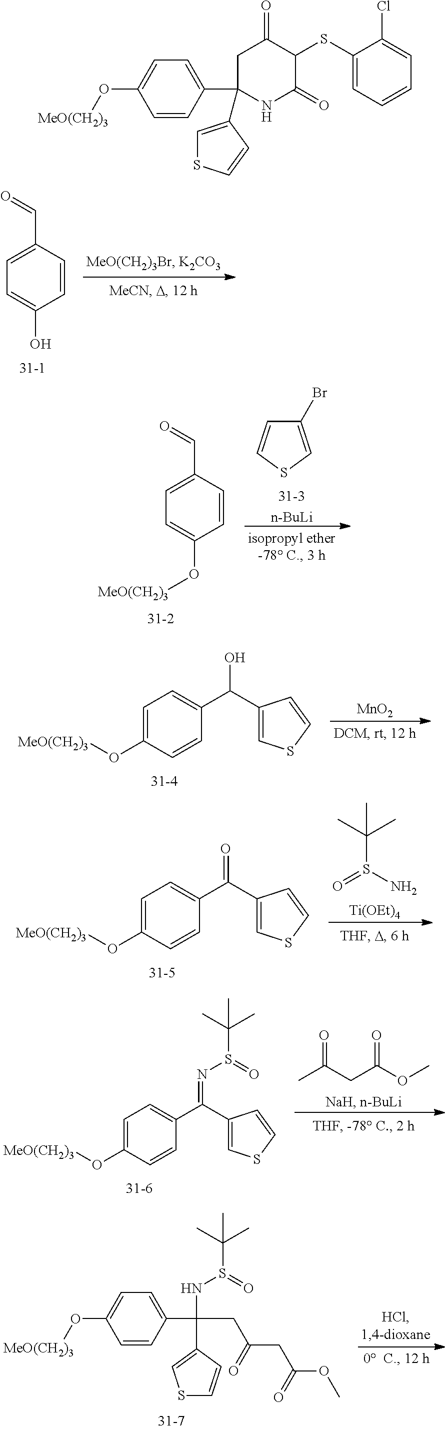

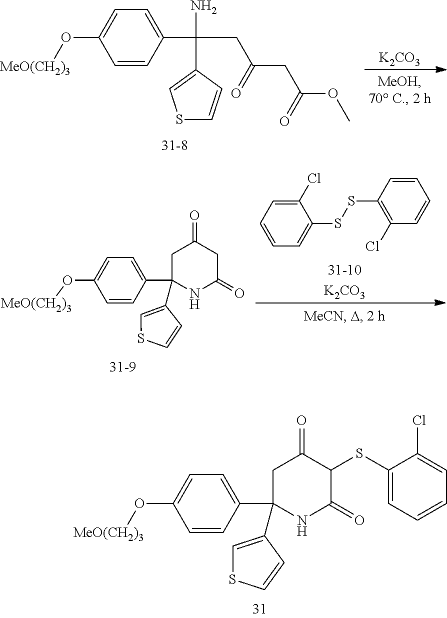

Step A: 4-(3-Methoxypropoxy)benzaldehyde was prepared in 96% yield according to the Example 27, Step A substituting (3-bromopropoxy)(tert-butyl)dimethylsilane for 1-bromo-3-methoxypropane.

Step B: (4-(3-Methoxypropoxy)phenyl)(thiophen-3-yl)methanol was prepared in 97% yield according to the Example 7, Step A substituting 4-bromobenzaldehyde for 4-(3-methoxypropoxy)benzaldehyde.

Step C: (4-(3-Methoxypropoxy)phenyl)(thiophen-3-yl)methanone was prepared in 68% yield according to the Example 7, Step B substituting (4-bromophenyl)(thiophen-3-yl)methanone for (4-(3-methoxypropoxy)phenyl) (thiophen-3-yl)methanol.

Step D: (Z)—N-((4-(3-Methoxypropoxy)phenyl)(thiophen-3-yl)methylene)-2-methylpropane-2-sulfinamide was prepared in 51% yield according to the Example 7, Step C substituting (E)-N-((4-bromophenyl) (thiophen-3-yl)methylene)-2-methylpropane-2-sulfinamide for (4-(3-methoxypropoxy)phenyl)(thiophen-3-yl)methanone.

Step E: methyl 5-(1,1-dimethylethylsulfinamido)-5-(4-(3-methoxypropoxy)phenyl)-3-oxo-5-(thiophen-3-yl)pentanoate was prepared in 93% yield according to the Example 7, Step D substituting (E)-N-((4-bromophenyl) (thiophen-3-yl)methylene)-2-methylpropane-2-sulfinamide for (Z)—N-((4-(3-methoxypropoxy)phenyl)(thiophen-3-yl)methylene)-2-methylpropane-2-sulfinamide.

Step F: methyl 5-amino-5-(4-(3-methoxypropoxy)phenyl)-3-oxo-5-(thiophen-3-yl)pentanoate in 90% yield according to the Example 7, Step E substituting methyl 5-(4-bromophenyl)-5-(1,1-dimethylethylsulfinamido)-3-oxo-5-(thiophen-3-yl)pentanoate for methyl 5-(1,1-dimethylethylsulfinamido)-5-(4-(3-methoxypropoxy)phenyl)-3-oxo-5-(thiophen-3-yl)pentanoate Step G: 6-(4-(3-Methoxypropoxy)phenyl)-6-(thiophen-3-yl)piperidine-2,4-dione was prepared in 33% yield according to the Example 7, Step F substituting methyl 5-amino-5-(4-bromophenyl)-3-oxo-5-(thiophen-3-yl)pentanoate for methyl 5-amino-5-(4-(3-methoxypropoxy)phenyl)-3-oxo-5-(thiophen-3-yl)pentanoate.

Step H: 3-((2-Chlorophenyl)thio)-6-(4-(3-methoxypropoxy)phenyl)-6-(thiophen-3-yl)piperidine-2,4-dione was prepared in 33% yield according to the Example 7, Step G substituting 6-(4-bromophenyl)-4-hydroxy-6-(thiophen-3-yl)-5,6-dihydropyridin-2(1H)-one for 6-(4-(3-methoxypropoxy)phenyl)-6-(thiophen-3-yl)piperidine-2,4-dione. ¹H NMR (400 MHz, (CD₃)₂SO) δ 8.41 (s, 1H), 7.54 (dd, J=5.1, 2.9 Hz, 1H), 7.25-7.30 (m, 4H), 7.13 (dd, J=5.1, 1.1 Hz, 1H), 6.88-6.95 (m, 3H), 6.68-6.72 (m, 1H), 5.83 (dd, J=7.9, 1.1 Hz, 1H), 4.00 (t, J=6.4 Hz, 2H), 3.44 (t, J=6.3 Hz, 2H), 3.21 (s, 2H), 3.25 (s, 3H), 1.90 (t, J=6.3 Hz, 2H). LCMS M+1=501.9.

### Example 32

### 3-(2-chlorophenyl)sulfanyl-6-(2-naphthyl)-6-(3-thienyl)piperidine-2,4-dione

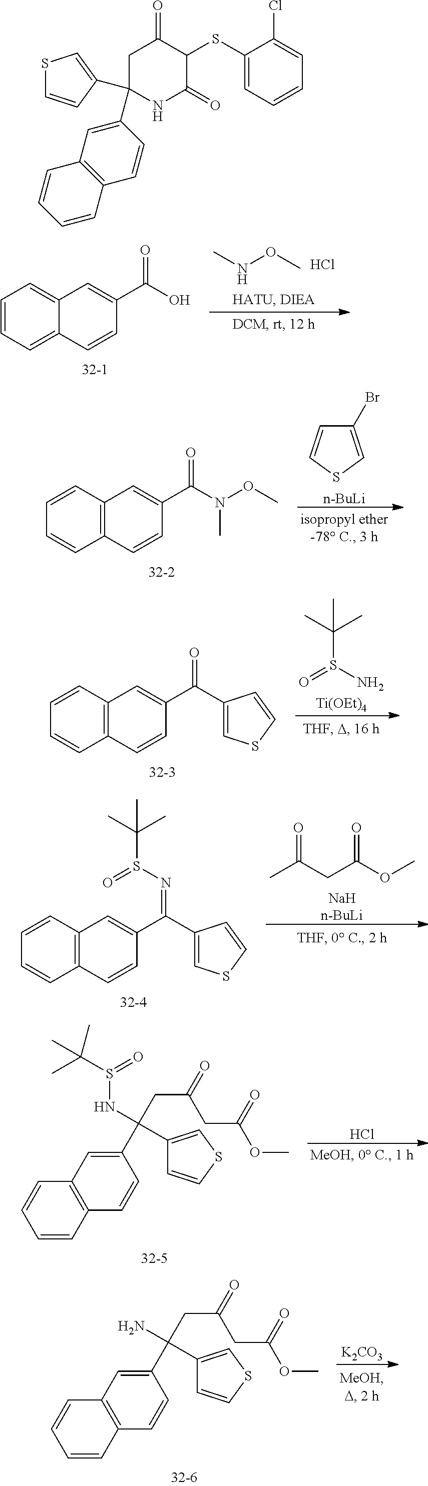

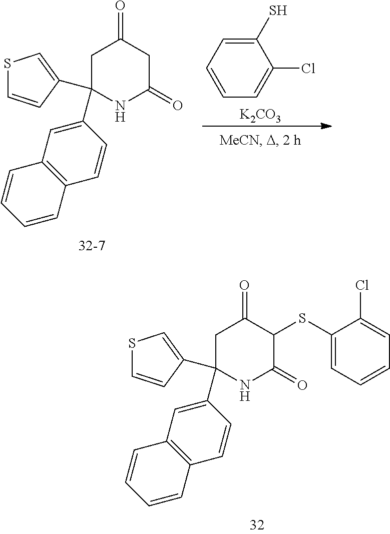

Step A: N-Methoxy-N-methyl-2-naphthamide was prepared in 90% yield according to the Example 1, Step A substituting 6-bromopicolinic acid for 2-naphthoic acid.

Step B: Naphthalen-2-yl(thiophen-3-yl)methanone was prepared in 25% yield according to the Example 1, Step B substituting 6-bromo-N-methoxy-N-methylpicolinamide for N-methoxy-N-methyl-2-naphthamide.

Step C: (E)-2-Methyl-N-(naphthalen-2-yl(thiophen-3-yl)methylene)propane-2-sulfinamide was prepared in 78% yield according to the Example 1, Step C substituting (6-bromopyridin-2-yl)(thiophen-3-yl)methanone for naphthalen-2-yl(thiophen-3-yl)methanone.

Step D: Methyl 5-(1,1-dimethylethylsulfinamido)-5-(naphthalen-2-yl)-3-oxo-5-(thiophen-3-yl)pentanoate was prepared in 80% yield according to the Example 1, Step D substituting (Z)—N-((6-bromopyridin-2-yl)(thiophen-3-yl)methylene)-2-methylpropane-2-sulfinamide for (E)-2-methyl-N-(naphthalen-2-yl(thiophen-3-yl)methylene)propane-2-sulfinamide.

Step E: Methyl 5-amino-5-(naphthalen-2-yl)-3-oxo-5-(thiophen-3-yl)pentanoate was prepared in 80% yield according to the Example 1, Step E substituting methyl 5-amino-5-(6-bromopyridin-2-yl)-3-oxo-5-(thiophen-3-yl)pentanoate for methyl 5-(1,1-dimethylethylsulfinamido)-5-(naphthalen-2-yl)-3-oxo-5-(thiophen-3-yl)pentanoate.

Step F: 4-Hydroxy-6-(naphthalen-2-yl)-6-(thiophen-3-yl)-5,6-dihydropyridin-2(1H)-one was prepared in 92% yield according to the Example 1, Step F substituting 6′-bromo-4-hydroxy-2-(thiophen-3-yl)-2,3-dihydro-[2,2′-bipyridin]-6(1H)-one for methyl 5-amino-5-(naphthalen-2-yl)-3-oxo-5-(thiophen-3-yl)pentanoate.

Step G: 3-((2-Chlorophenyl)thio)-4-hydroxy-6-(naphthalen-2-yl)-6-(thiophen-3-yl)-5,6-dihydropyridin-2(1H)-one was prepared in 9% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for 4-hydroxy-6-(naphthalen-2-yl)-6-(thiophen-3-yl)-5,6-dihydropyridin-2(1H)-one. ¹H NMR (400 MHz, (CD₃)₂SO) δ 11.53 (s, 1H), 8.65 (s, 1H), 7.69-7.62 (m, 4H), 7.61-7.52 (m, 4H), 7.41 (dd, J=2.8, 1.2 Hz, 1H), 7.26-7.22 (m, 2H), 6.88-6.84 (m, 1H), 6.30-5.79 (m, 1H), 5.77 (d, J=8.0 Hz, 1H), 3.61 (d, J=16.8 Hz, 1H), 3.61 (d, J=16.8 Hz, 1H). LCMS 463.8.

### Example 33

### 3-(2-chlorophenyl)sulfanyl-6-(4-cyclopropylphenyl)-6-(3-thienyl)piperidine-2,4-dione

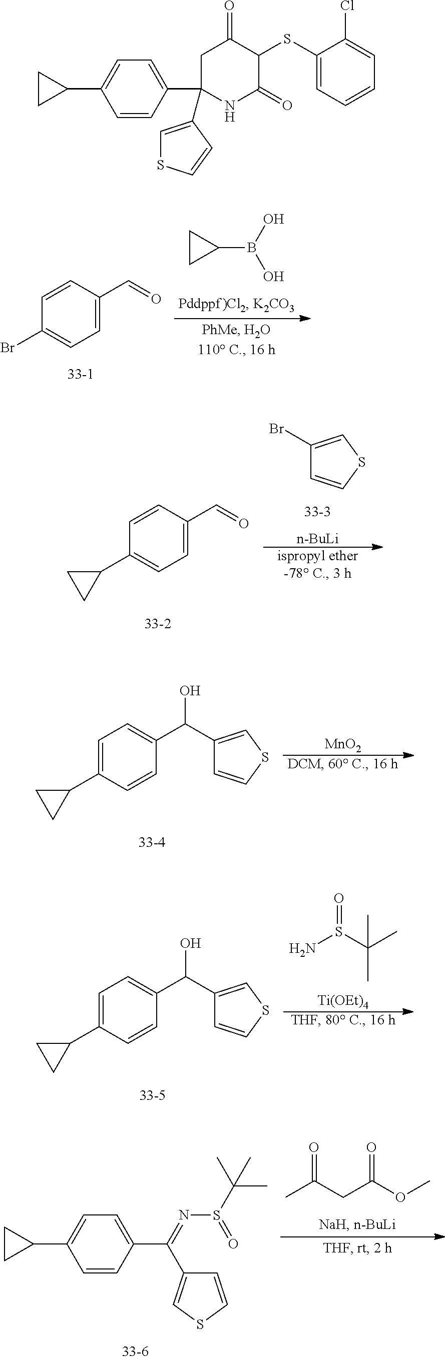

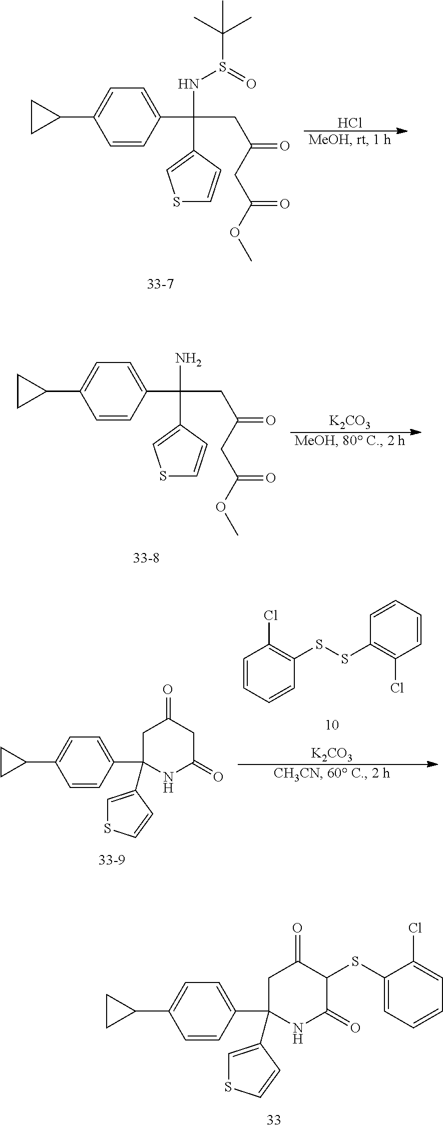

Step A: 4-Cyclopropylbenzaldehyde was prepared in 80% yield according to the Example 8, Step A substituting cyclohex-1-en-1-ylboronic acid for cyclopropylboronic acid and 6-(4-bromophenyl)-3-((2-chlorophenyl)thio)-6-(thiophen-3-yl) piperidine-2,4-dione for 4-bromobenzaldehyde.

Step B: (4-Cyclopropylphenyl)(thiophen-3-yl)methanol was prepared in 91% yield according to the Example 7, Step A substituting 4-bromobenzaldehyde for 4-cyclopropylbenzaldehyde.

Step C: (4-Cyclopropylphenyl)(thiophen-3-yl)methanone was prepared in 88% yield according to the Example 7, Step B substituting (4-bromophenyl)(thiophen-3-yl)methanol for (4-cyclopropylphenyl)(thiophen-3-yl)methanol.

Step D: (Z)—N-((4-Cyclopropylphenyl)(thiophen-3-yl)methylene)-2-methylpropane-2-sulfinamide was prepared in 71% yield according to the Example 1, Step C substituting (6-bromopyridin-2-yl)(thiophen-3-yl)methanone for (4-cyclopropylphenyl)(thiophen-3-yl)methanone

Step E: Methyl 5-(4-cyclopropylphenyl)-5-(1,1-dimethylethylsulfinamido)-3-oxo-5-(thiophen-3-yl)pentanoate was prepared in 98% yield according to the Example 1, Step D substituting (Z)—N-((6-bromopyridin-2-yl)(thiophen-3-yl)methylene)-2-methylpropane-2-sulfinamide for (Z)—N-((4-cyclopropylphenyl)(thiophen-3-yl)methylene)-2-methylpropane-2-sulfinamide.

Step F: Methyl 5-amino-5-(4-cyclopropylphenyl)-3-oxo-5-(thiophen-3-yl)pentanoate was prepared in 72% yield according to the Example 1, Step E substituting methyl 5-amino-5-(6-bromopyridin-2-yl)-3-oxo-5-(thiophen-3-yl)pentanoate for methyl 5-(4-cyclopropylphenyl)-5-(1,1-dimethylethylsulfinamido)-3-oxo-5-(thiophen-3-yl)pentanoate.

Step G: 6-(4-Cyclopropylphenyl)-6-(thiophen-3-yl)piperidine-2,4-dione was prepared in 55% yield according to the Example 1, Step F substituting 6′-bromo-4-hydroxy-2-(thiophen-3-yl)-2,3-dihydro-[2,2′-bipyridin]-6(1H)-one for methyl 5-amino-5-(4-cyclopropylphenyl)-3-oxo-5-(thiophen-3-yl)pentanoate.

Step H: 3-((2-Chlorophenyl)thio)-6-(4-cyclopropylphenyl)-6-(thiophen-3-yl)piperidine-2,4-dione was prepared in 38% yield according to the Example 1, Step G substituting 6′-bromo-5-((2-chlorophenyl)thio)-4-hydroxy-2-(thiophen-3-yl)-2,3-dihydro-[2,2′-bipyridin]-6(1H)-one for 6-(4-cyclopropylphenyl)-6-(thiophen-3-yl)piperidine-2,4-dione. ¹H NMR (400 MHz, (CD₃)₂SO) δ 7.47 (dd, J=8.0, 8.0 Hz, 1H), 7.31-7.27 (m, 3H), 7.18 (dd, J=4.0, 4.0 Hz, 1H), 7.11 (dd, J=8.0, 4.0 Hz, 1H), 7.02 (d, J=8.0 Hz, 2H), 6.84 (dd, J=8.0, 8.0 Hz, 1H), 6.67 (dd, J=8.0, 8.0 Hz, 1H), 5.98 (d, J=8.0 Hz, 1H), 3.24 (d, J=5.2 Hz, 2H), 1.92-1.86 (m, 1H), 0.96-0.91 (m, 2H), 0.67-0.63 (m, 2H). LCMS M+1=453.8.

### Example 34

### 3-(2-chlorophenyl)sulfanyl-1-methyl-6-[3-(tetrahydropyran-4-ylamino)phenyl]-6-(3-thienyl)piperidine-2,4-dione

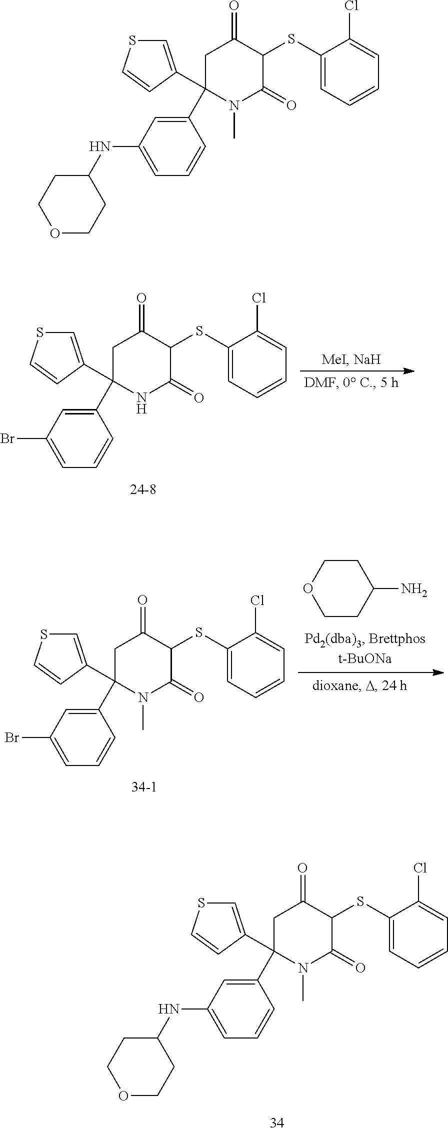 [SMILES: CN1C(=O)C(Sc2ccccc2Cl)C(=O)CC1(c1ccsc1)c1cccc(NC2CCOCC2)c1 ; O=C1CC(c2ccsc2)(c2cccc(Br)c2)NC(=O)C1Sc1ccccc1Cl ; CN1C(=O)C(Sc2ccccc2Cl)C(=O)CC1(c1ccsc1)c1cccc(Br)c1 ; NC1CCOCC1 ; CN1C(=O)C(Sc2ccccc2Cl)C(=O)CC1(c1ccsc1)c1cccc(NC2CCOCC2)c1]

Step A: 6-(3-Bromophenyl)-3-((2-chlorophenyl)thio)-4-hydroxy-1-methyl-6-(thiophen-3-yl)-5,6-dihydropyridin-2(1H)-one was prepared by Example 11 in 15% yield, step A substituting 6′-bromo-5-((2-chlorophenyl)thio)-4-hydroxy-2-(thiophen-3-yl)-2,3-dihydro-[2,2′-bipyridin]-6(1H)-one for 6-(3-bromophenyl)-3-((2-chlorophenyl)thio)-4-hydroxy-6-(thiophen-3-yl)-5,6-dihydropyridin-2 (1H)-one.

Step B: 3-(2-Chlorophenyl)sulfanyl-1-methyl-6-[3-(tetrahydropyran-4-ylamino)phenyl]-6-(3-thienyl)piperidine-2,4-dione was prepared in 6% yield, according to Example 7. Step H substituting 2-methylmorpholine for tetrahydro-2H-pyran-4-amine. ¹H NMR (400 MHz, (CD₃)₂SO) δ 11.3 (s, 1H), 7.67 (dd, J=5.2, 3.2 Hz, 1H), 7.31 (dd, J=8.0, 1.6 Hz, 1H), 7.15-7.10 (m, 3H), 6.98 (dd, J=7.6, 1.2 Hz, 1H), 6.88 (dd, J=7.6, 1.2 Hz, 1H), 6.61 (dd, J=8.4, 1.6 Hz, 1H), 6.43 (m, 2H), 6.15 (d, J=8.4 Hz, 1H), 3.85 (m, 2H), 3.60 (d, J=16.8 Hz, 1H), 3.48 (d, J=16.8 Hz, 1H), 3.44 (m, 3H), 2.69 (s, 3H), 1.82 (m, 2H), 1.34 (m, 2H). LCMS M+1=527.0.

### Example 35

### 3-(2-chlorophenyl)sulfanyl-6-(2-hydroxy-4-morpholino-phenyl)-6-(3-thienyl)piperidine-2,4-dione

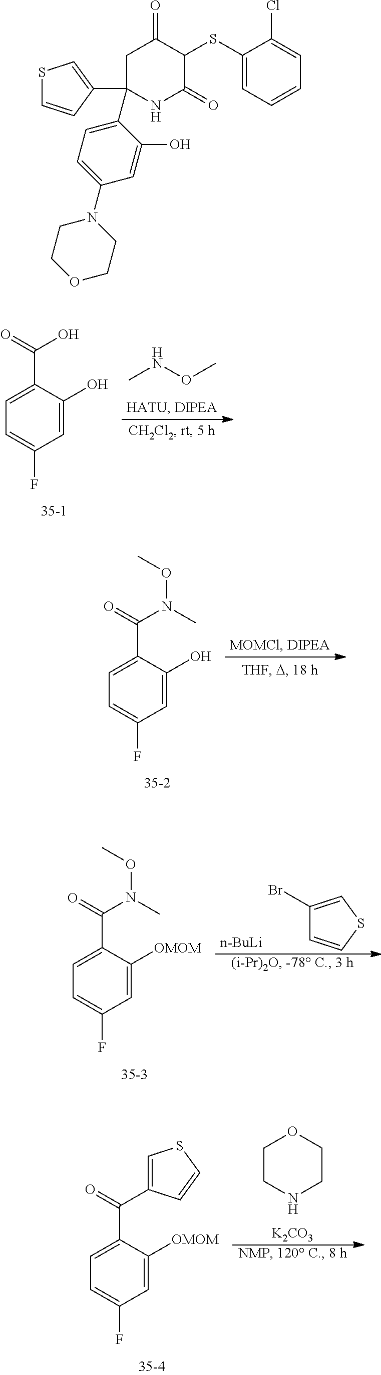

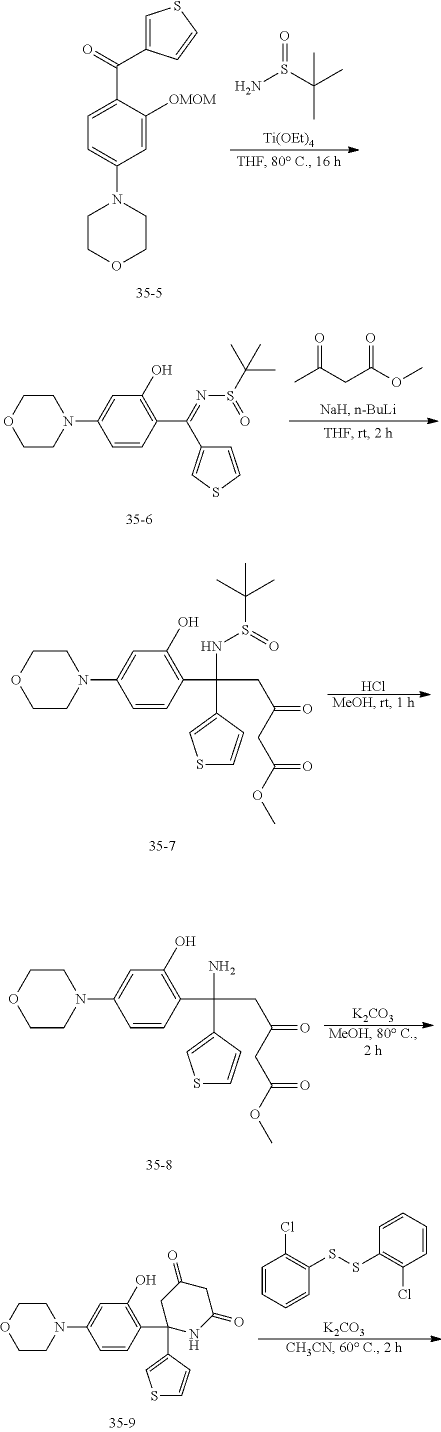

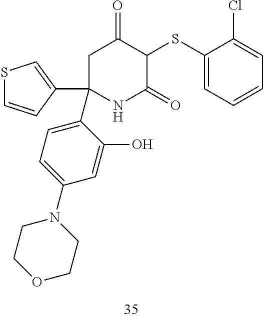 [SMILES: O=C1CC(c2ccsc2)(c2ccc(N3CCOCC3)cc2O)NC(=O)C1Sc1ccccc1Cl]

Step A: 4-Fluoro-2-hydroxy-N-methoxy-N-methylbenzamide was prepared in 75% yield according to the Example 1, Step A substituting 6-bromopicolinic acid for 4-fluoro-2-hydroxybenzoic acid.

Step B: 4-Fluoro-N-methoxy-2-(methoxymethoxy)-N-methylbenzamide was prepared in 62% yield according to Example 13, Step A substituting 5-bromo-2-hydroxybenzaldehyde for 4-fluoro-2-hydroxy-N-methoxy-N-methylbenzamide.

Step C: (4-Fluoro-2-(methoxymethoxy)phenyl)(thiophen-3-yl)methanone was prepared in 41% yield according to the Example 1, Step B substituting 6-bromo-N-methoxy-N-methylpicolinamide for 4-fluoro-N-methoxy-2-(methoxymethoxy)-N-methylbenzamide.

Step D: To a solution of (4-fluoro-2-(methoxymethoxy)phenyl)(thiophen-3-yl)methanone (10 g, 38 mmol) in NMP (50 mL) was added morpholine (16.4 g, 188 mmol) and K₂CO₃ (10.4 g, 75 mmol). The solution was stirred at 120° C. for 8 hours. The reaction was quenched with water, adjusted to pH=5 with HCl solution, extracted with DCM, and concentrated under vacuum. The crude residue was purified by silica gel column to afford (6-(4-fluorophenoxy)pyridin-2-yl)(2-(methoxymethoxy)phenyl)methanone (7.2 g, 60% yield).

Step E: N-((2-Hydroxy-4-morpholinophenyl)(thiophen-3-yl)methylene)-2-methylpropane-2-sulfinamide was prepared in 10% yield according to the Example 1, Step C substituting (6-bromopyridin-2-yl)(thiophen-3-yl)methanone for (6-(4-fluorophenoxy)pyridin-2-yl) (2-(methoxymethoxy)phenyl)methanone.

Step F: Methyl 5-(1,1-dimethylethylsulfinamido)-5-(2-hydroxy-4-morpholinophenyl)-3-oxo-5-(thiophen-3-yl)pentanoate was prepared in 36% yield according to the Example 1, Step D substituting N-((6-bromopyridin-2-yl) (thiophen-3-yl)methylene)-2-methylpropane-2-sulfinamide for N-((2-hydroxy-4-morpholinophenyl)(thiophen-3-yl)methylene)-2-methylpropane-2-sulfinamide.

Step G: Methyl 5-amino-5-(2-hydroxy-4-morpholinophenyl)-3-oxo-5-(thiophen-3-yl)pentanoate was prepared in 40% yield according to the Example 1, Step E substituting methyl 5-(6-bromopyridin-2-yl)-5-(1,1-dimethylethylsulfinamido)-3-oxo-5-(thiophen-3-yl)pentanoate for methyl 5-(1,1-dimethylethylsulfinamido)-5-(2-hydroxy-4-morpholinophenyl)-3-oxo-5-(thiophen-3-yl)pentanoate.

Step H: 3-(2-Chlorophenyl)sulfanyl-6-(2-hydroxy-4-morpholino-phenyl)-6-(3-thienyl) piperidine-2,4-dione was prepared in 2% yield, according to Example 1, Step G substituting 6′-bromo-4-hydroxy-2-(thiophen-3-yl)-2,3-dihydro-[2,2′-bipyridin]-6(1H)-one as methyl 5-amino-5-(2-hydroxy-4-morpholinophenyl)-3-oxo-5-(thiophen-3-yl)pentanoate. ¹H NMR (400 MHz, (CD₃)₂SO) δ 7.36 (d, J=5.2 Hz, 1H), 7.20-7.15 (m, 3H), 7.06 (d, J=5.2 Hz, 1H), 6.92 (dd, J=7.6, 1.6 Hz, 1H), 6.77 (dd, J=7.6, 1.6 Hz, 1H), 6.50 (m, 2H), 6.19 (d, J=8.0 Hz, 1H), 3.83 (dd, J=4.8, 4.8 Hz, 4H), 3.68 (d, J=16.4 Hz, 1H), 3.36 (d, J=16.4 Hz, 1H), 3.13 (dd, J=4.8, 4.8 Hz, 4H). LCMS M+1=514.9.

### Example 36

### 3-(2-chlorophenyl)sulfanyl-6-(2-hydroxyphenyl)-6-(3-thienyl)piperidine-2,4-dione

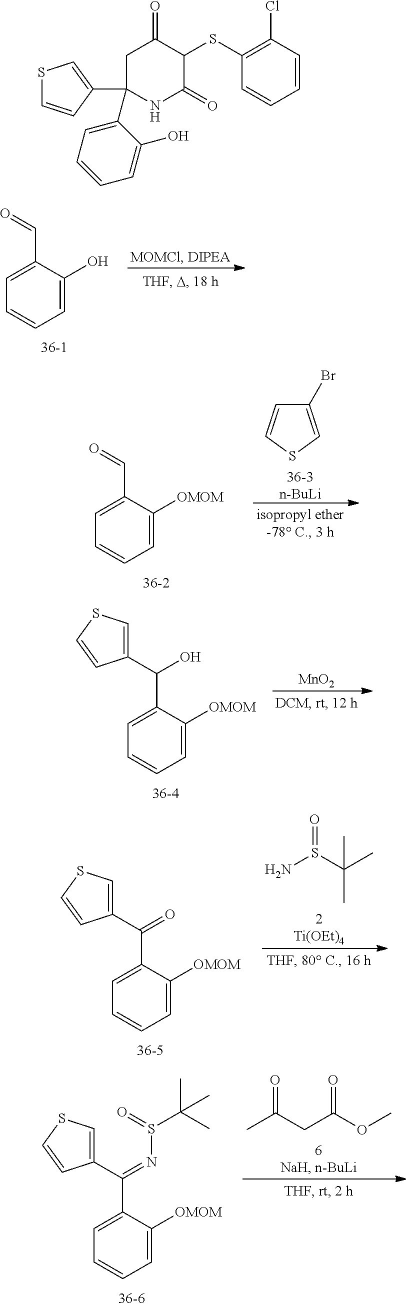

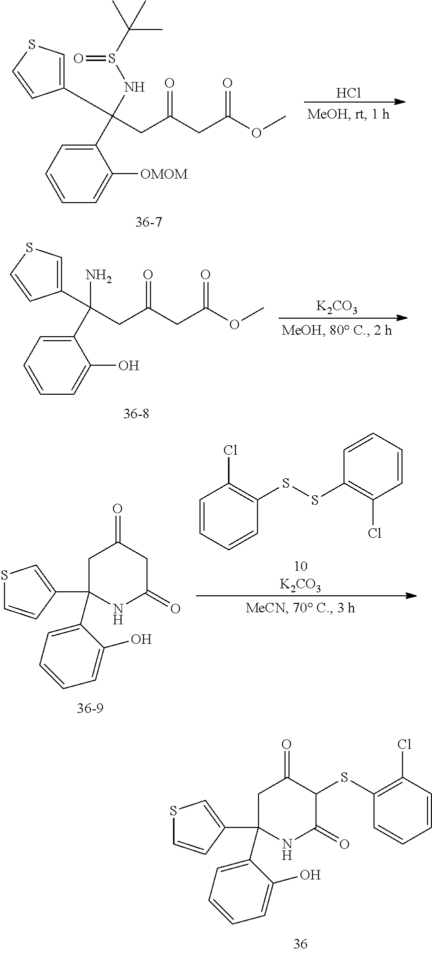

Step A: 2-(Methoxymethoxy)benzaldehyde was prepared in 82% according to Example 13, Step A substituting 5-bromo-2-hydroxybenzaldehyde for 2-hydroxybenzaldehyde.

Step B: (2-(Methoxymethoxy)phenyl)(thiophen-3-yl)methanol was prepared in 60% yield according to the Example 7, Step A substituting 4-bromobenzaldehyde for 2-(methoxymethoxy)benzaldehyde.

Step C: (2-(Methoxymethoxy)phenyl)(thiophen-3-yl)methanone was prepared in 71% yield according to the Example 7, Step B substituting (4-bromophenyl)(thiophen-3-yl)methanol for (2-(methoxymethoxy)phenyl)(thiophen-3-yl)methanol.

Step D: N-((2-(Methoxymethoxy)phenyl)(thiophen-3-yl)methylene)-2-methylpropane-2-sulfinamide was prepared in 50% yield according to the Example 1, Step C substituting (6-bromopyridin-2-yl)(thiophen-3-yl)methanone for (2-(methoxymethoxy)phenyl) (thiophen-3-yl)methanone.

Step E: Methyl 5-(1,1-dimethylethylsulfinamido)-5-(2-(methoxymethoxy)phenyl)-3-oxo-5-(thiophen-3-yl)pentanoate was prepared in 47% yield according to the Example 1, Step D substituting N-((6-bromopyridin-2-yl)(thiophen-3-yl)methylene)-2-methylpropane-2-sulfinamide for N-((2-(methoxymethoxy)phenyl)(thiophen-3-yl)methylene)-2-methylpropane-2-sulfinamide.

Step F: Methyl 5-amino-5-(2-hydroxyphenyl)-3-oxo-5-(thiophen-3-yl)pentanoate was prepared in 51% yield according to the Example 1, Step E substituting methyl 5-(6-bromopyridin-2-yl)-5-(1,1-dimethylethylsulfinamido)-3-oxo-5-(thiophen-3-yl)pentanoate for methyl 5-(1,1-dimethylethylsulfinamido)-5-(2-(methoxymethoxy)phenyl)-3-oxo-5-(thiophen-3-yl)pentanoate.

Step G: 6-(2-Hydroxyphenyl)-6-(thiophen-3-yl) piperidine-2,4-dione was prepared in 60% yield according to the Example 1, Step F substituting methyl 5-amino-5-(6-bromopyridin-2-yl)-3-oxo-5-(thiophen-3-yl)pentanoate for methyl 5-amino-5-(2-hydroxyphenyl)-3-oxo-5-(thiophen-3-yl)pentanoate.

Step H: 3-((2-Chlorophenyl)thio)-4-hydroxy-6-(2-hydroxyphenyl)-6-(thiophen-3-yl)-5,6-dihydropyridin-2(1H)-one was prepared in 7% yield according to the Example 1, Step G substituting 6′-bromo-4-hydroxy-2-(thiophen-3-yl)-2,3-dihydro-[2,2′-bipyridin]-6(1H)-one for 6-(2-hydroxyphenyl)-6-(thiophen-3-yl) piperidine-2,4-dione. ¹H NMR (400 MHz, (CD₃)₂SO) δ 9.81 (s, 1H), 7.69 (s, 1H), 7.46 (d, J=5.2 Hz, 1H), 7.29 (m, 3H), 7.26 (m, 1H), 7.17 (m, 1H), 6.96 (m, 1H), 6.86 (m, 2H), 6.74 (m, 1H), 6.10 (d, J=8.0 Hz, 1H), 3.74 (d, J=16.4 Hz, 1H), 3.42 (d, J=16.4 Hz, 1H). LCMS M+1=429.8.

### Example 37

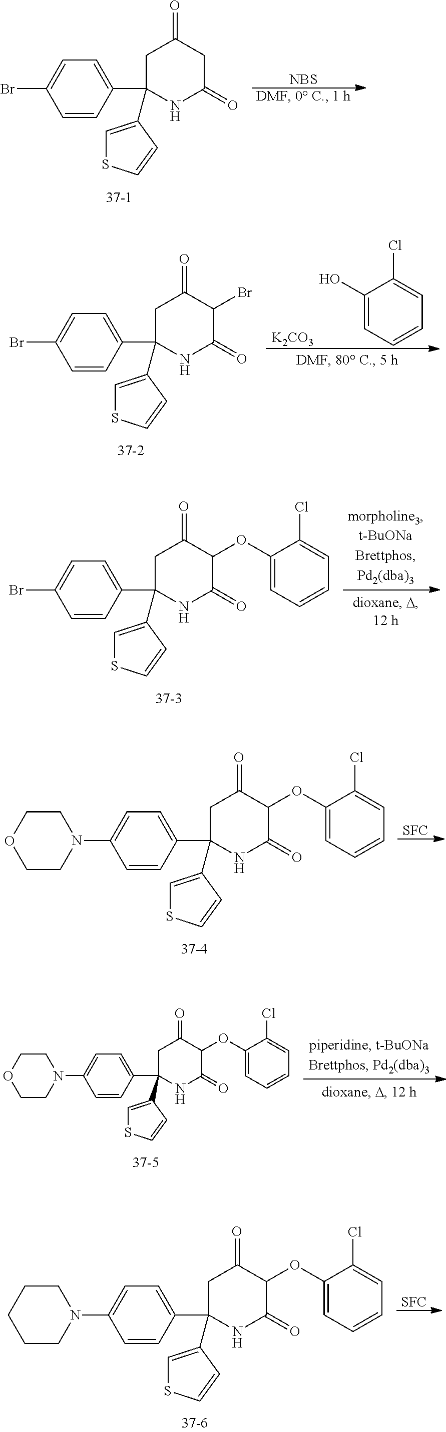

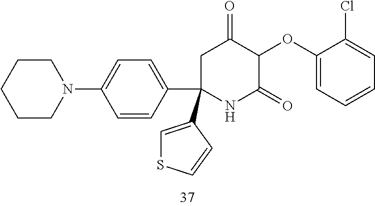 [SMILES: O=C1C[C@](c2ccc(N3CCCCC3)cc2)(c2ccsc2)NC(=O)C1Oc1ccccc1Cl]

Step A: To a stirred solution of 6-(4-bromophenyl)-6-(thiophen-3-yl)piperidine-2,4-dione (5 g, 14.3 mmol) in DMF (50 mL) was added NBS (3.05 g, 17.8 mol) in an ice bath. The reaction was stirred at 0° C. for 30 min. The reaction mixture was used in next step directly.

Step B: The solution of 3-bromo-6-(4-bromophenyl)-6-(thiophen-3-yl) piperidine-2,4-dione (14.3 mmol) in DMF (50 mL) was added 2-chlorophenol (2.8 g, 21.5 mmol) and potassium carbonate (5.9 g, 42.9 mmol). The reaction was stirred at 80° C. for 12 hours. The reaction mixture was extracted with EtOAc and brine. The organic layer was dried and concentrated. The crude was purified by chromatography on silica gel (PE/EA=2/1) to afford 6-(4-bromophenyl)-3-(2-chlorophenoxy)-6-(thiophen-3-yl)piperidine-2,4-dione (2 g, 4.2 mmol, 29%) as light color solid.

Step C: In a solution of 6-(4-bromophenyl)-3-(2-chlorophenoxy)-6-(thiophen-3-yl) piperidine-2,4-dione (600 mg, 1.26 mmol) in dioxane (10 mL) was added morpholine (328 mg, 3.77 mmol), Brettphos (65 mg, 0.13 mmol), Pd₂(dba)₃ (64 mg, 0.07 mmol) and t-BuONa (362 mg, 3.77 mmol). The solution was stirred for 8 h at 110° C. under nitrogen. The solvent was removed under vacuum and the residue was purified by Prep-HPLC (FA) and SFC to afford (6S)-3-(2-chlorophenoxy)-6-(4-morpholinophenyl)-6-(thiophen-3-yl)piperidine-2,4-dione (35 mg, 6%) as white solid.

Step D: (6S)-3-(2-chlorophenoxy)-6-(4-(piperidin-1-yl)phenyl)-6-(thiophen-3-yl)piperidine-2,4-dione was prepared in 8% yield according to the Method 37, Step C substituting morpholine for piperidine.

### Example 38

### 6-(6-bromopyridin-2-yl)-3-((2-chlorophenyl)thio)-6-(4-morpholinophenyl)piperidine-2,4-dione

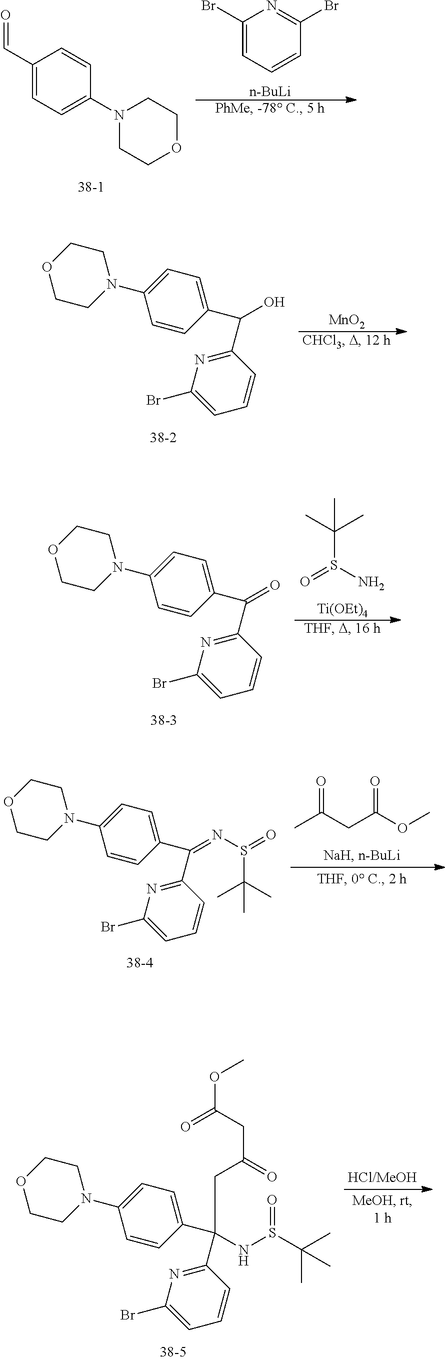

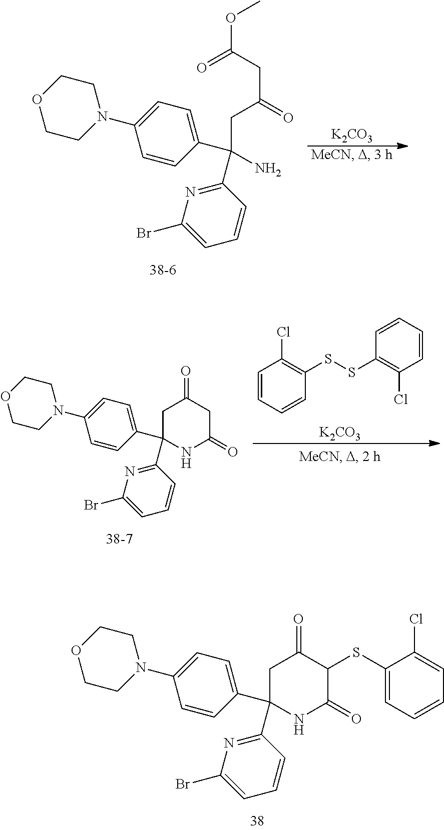

Step A: To a solution of 2,6-dibromopyridine (8.39 g, 31.4 mmol) in isopropyl ether (500 mL) was added n-BuLi (12.6 ml, 31.4 mmol) at −78° C. under N₂ protection. Then the mixture was stirred for 1 h. 4-Morpholinobenzaldehyde (5 g, 26.2 mmol) was added to above solution and the mixture was stirred at −78° C. for 2 h. TLC showed the reaction was completed. The mixture was quenched with MeOH and acidified to pH 4 with 1 N HCl, extracted with DCM (100 mL×2). The combined organic lays were dried over Na₂SO₄ and the crude product was purified by silica gel chromatography (PE:EA=3:1) to give the desired product (6.8 g, 79%) as a yellow oil.

Step B: (6-Bromopyridin-2-yl)(4-morpholinophenyl)methanone was prepared in 69% yield according to the method 7 Step B substituting (4-bromophenyl) (thiophen-3-yl)methanol for (6-bromopyridin-2-yl) (4-morpholinophenyl)methanol.

Step C: (Z)—N-((6-Bromopyridin-2-yl)(4-morpholinophenyl) methylene)-2-methylpropane-2-sulfinamide was prepared in 58% yield according to the Method 1, Step C substituting (6-bromopyridin-2-yl)(thiophen-3-yl) methanone for (6-bromopyridin-2-yl)(4-morpholinophenyl)methanone.

Step D: methyl 5-(6-bromopyridin-2-yl)-5-(1,1-dimethylethylsulfinamido)-5-(4-morpholinophenyl)-3-oxopentanoate was prepared in 79% yield according to the Method 1, Step D substituting (Z)—N-((6-bromopyridin-2-yl)(thiophen-3-yl) methylene)-2-methylpropane-2-sulfinamide for (Z)—N-((6-bromopyridin-2-yl) (4-morpholinophenyl)methylene)-2-methylpropane-2-sulfinamide.

Step E: methyl 5-amino-5-(6-bromopyridin-2-yl)-5-(4-morpholinophenyl)-3-oxopentanoate was prepared in 67% yield according to the Method 1, Step E substituting methyl 5-amino-5-(6-bromopyridin-2-yl)-3-oxo-5-(thiophen-3-yl) pentanoate for methyl 5-(6-bromopyridin-2-yl)-5-(1,1-dimethylethylsulfinamido)-5-(4-morpholinophenyl)-3-oxopentanoate.

Step F: 6′-bromo-4-hydroxy-2-(4-morpholinophenyl)-2,3-dihydro-[2,2′-bipyridin]-6(1H)-one was prepared in 47% yield according to the Method 1, Step F substituting 6′-bromo-4-hydroxy-2-(thiophen-3-yl)-2,3-dihydro-[2,2′-bipyridin]-6(1H)-one for methyl 5-amino-5-(6-bromopyridin-2-yl)-5-(4-morpholinophenyl)-3-oxopentanoate.

Step G: 6′-bromo-5-((2-chlorophenyl)thio)-4-hydroxy-2-(4-morpholinophenyl)-2,3-dihydro-[2,2′-bipyridin]-6(1H)-one was prepared in 96% yield according to the Method 1, Step G substituting 6′-bromo-5-((2-chlorophenyl)thio)-4-hydroxy-2-(thiophen-3-yl)-2,3-dihydro-[2,2′-bipyridin]-6(1H)-one for 6′-bromo-4-hydroxy-2-(4-morpholinophenyl)-2,3-dihydro-[2,2′-bipyridin]-6(1H)-one.

### Example 39

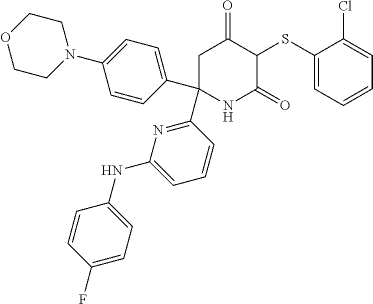 [SMILES: O=C1CC(c2ccc(N3CCOCC3)cc2)(c2cccc(Nc3ccc(F)cc3)n2)NC(=O)C1Sc1ccccc1Cl]

Step A: 3-((2-chlorophenyl)thio)-6-(6-((4-fluorophenyl)amino) pyridin-2-yl)-6-(4-morpholinophenyl)piperidine-2,4-dione was prepared in 42% yield according to the Method 4, Step A substituting 6-(6-bromopyridin-2-yl)-3-((2-chlorophenyl) thio)-6-(thiophen-3-yl) piperidine-2,4-dione for 6-(6-bromopyridin-2-yl)-3-((2-chlorophenyl)thio)-6-(4-morpholinophenyl) piperidine-2,4-dione and cyclohexanamine for 4-fluoroaniline. 1H NMR (400 MHz, METHANOL-d4) d=7.57 (dd, J=8.0, 8.0 Hz, 1H), 7.54-7.50 (m, 2H), 7.36 (d, J=9.2, 2H), 7.20-6.92 (m, 6H), 6.73-6.67 (m, 2H), 6.12 (d, J=7.2 Hz, 1H), 3.84 (dd, J=9.2, 4.4 Hz, 4H), 3.75 (d, J=16.4 Hz, 1H), 3.49 (d, J=16.4 Hz, 1H), 3.16 (dd, J=9.2, 4.4 Hz, 4H), single stereoisomer. 1H NMR (400 MHz, METHANOL-d4) d=7.51 (dd, J=8.0, 8.0 Hz, 1H), 7.49-7.47 (m, 2H), 7.33 (d, J=8.8, 2H), 7.18 (d, J=8.0, 1H), 6.99-6.89 (m, 6H), 6.71-6.69 (m, 2H), 6.07 (dd, J=6.4, 1.6 Hz, 1H), 3.81 (dd, J=4.8, 4.8 Hz, 4H), 3.78 (d, J=16.8 Hz, 1H), 3.47 (d, J=16.8 Hz, 1H), 3.15 (dd, J=4.8, 4.8 Hz, 4H), mixture of diastereoisomers.

### Example 40

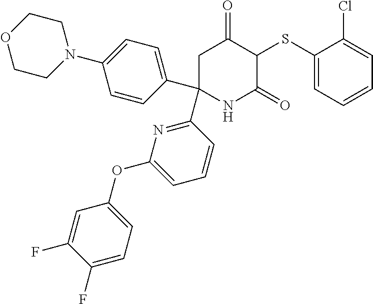 [SMILES: O=C1CC(c2ccc(N3CCOCC3)cc2)(c2cccc(Oc3ccc(F)c(F)c3)n2)NC(=O)C1Sc1ccccc1Cl]

Step A: 3-(2-chlorophenyl)sulfanyl-6-[6-(3,4-difluorophenoxy)-2-pyridyl]6-(4-morpholinophenyl)piperidine-2,4-dione was prepared in 47% yield according to the Method 3, Step A substituting 6-(6-bromopyridin-2-yl)-3-((2-chlorophenyl) thio)-6-(thiophen-3-yl)piperidine-2,4-dione for 6-(6-bromopyridin-2-yl)-3-((2-chlorophenyl)thio)-6-(4-morpholinophenyl)piperidine-2,4-dione and 2-Chloro-4-fluoro-phenol for 3,4-difluorophenol. 1H NMR (400 MHz, METHANOL-d4) d=7.88 (dd, J=8.0, 8.0 Hz, 1H), 7.33 (d, J=7.2, 1H), 7.20-7.16 (m, 4H), 7.02-6.92 (m, 5H), 6.85 (d, J=7.2, 1H), 6.72 (dd, J=8.0, 8.0, 1H), 5.91 (dd, J=8.0, 1.2 Hz, 1H), 3.81 (dd, J=4.8, 4.8 Hz, 4H), 3.55 (d, J=16.8 Hz, 1H), 3.31 (d, J=16.8 Hz, 1H), 3.12 (dd, J=4.8, 4.8 Hz, 4H), mixture of diastereoisomers. 1H NMR (400 MHz, METHANOL-d4) d=7.91 (dd, J=7.6, 7.6 Hz, 1H), 7.37 (d, J=7.6, 1H), 7.36-7.21 (m, 4H), 7.04-6.96 (m, 2H), 6.93-6.85 (m, 4H), 6.75 (dd, J=7.6, 7.6, 1H), 3.84 (dd, J=4.8, 4.8 Hz, 4H), 3.55 (d, J=16.8 Hz, 1H), 3.33 (d, J=16.8 Hz, 1H), 3.16 (dd, J=4.8, 4.8 Hz, 4H), single stereoisomer.

### Example 41

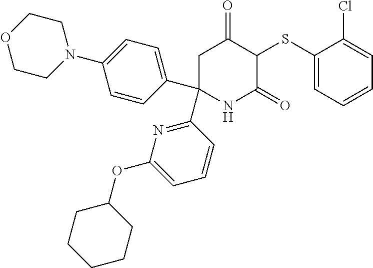 [SMILES: O=C1CC(c2ccc(N3CCOCC3)cc2)(c2cccc(OC3CCCCC3)n2)NC(=O)C1Sc1ccccc1Cl]

Step A: 3-((2-chlorophenyl)thio)-6-(6-(cyclohexyloxy)pyridin-2-yl)-6-(4-morpholinophenyl)piperidine-2,4-dione was prepared in 11% yield according to the Method 4, Step A substituting 6-(6-bromopyridin-2-yl)-3-((2-chlorophenyl)thio)-6-(thiophen-3-yl)piperidine-2,4-dione for 6-(6-bromopyridin-2-yl)-3-((2-chlorophenyl) thio)-6-(4-morpholinophenyl)piperidine-2,4-dione and propan-2-ol for cyclohexanol.

### Example 42

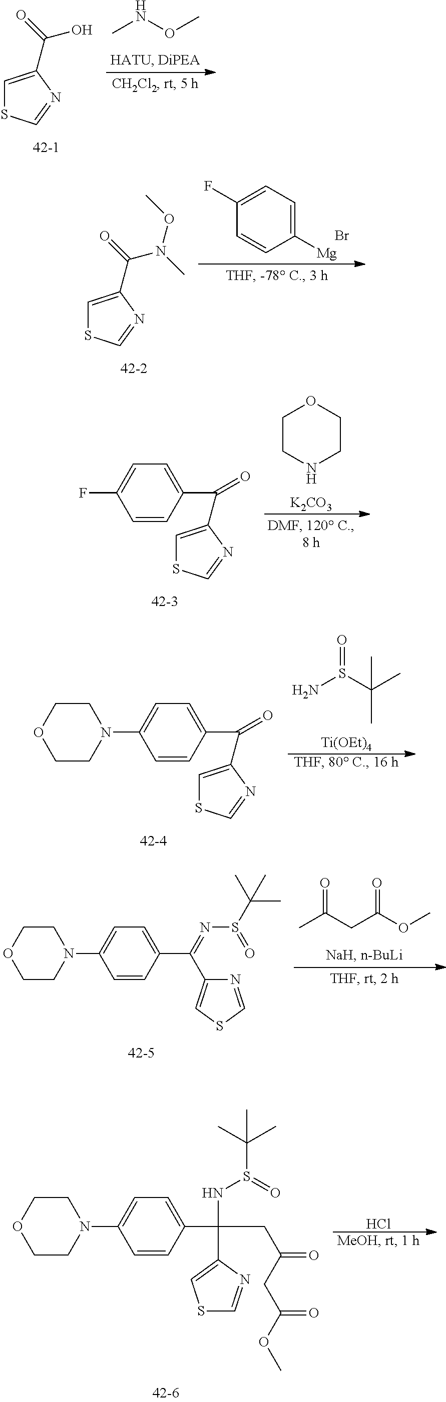

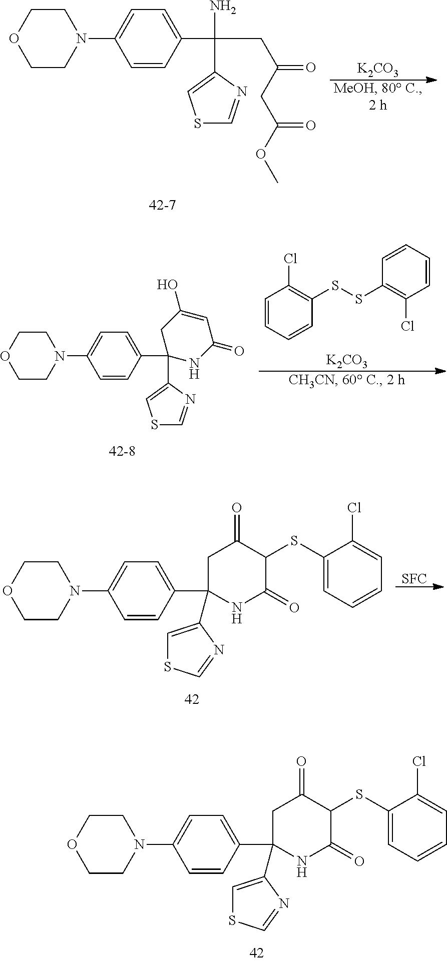

Step A: N-methoxy-N-methylthiazole-4-carboxamide was prepared in 67% yield according to the Method 1, Step A substituting 6-bromopicolinic acid for thiazole-4-carboxylic acid.

Step B: (4-fluorophenyl)(thiazol-4-yl)methanone was prepared in 72% yield according to the Method 36, Step A substituting 4-bromothiophene-2-carbaldehyde for N-methoxy-N-methylthiazole-4-carboxamide.

Step C: (4-morpholinophenyl)(thiazol-4-yl)methanone was prepared in 56% yield according to the Method 34, Step D substituting (4-fluoro-2-(methoxymethoxy)phenyl)(thiophen-3-yl)methanone for (4-fluorophenyl)(thiazol-4-yl)methanone.

Step D: (Z)-2-methyl-N-((4-morpholinophenyl)(thiazol-4-yl)methylene) propane-2-sulfinamide was prepared in 56% yield according to the Method 1, Step C substituting (6-bromopyridin-2-yl)(thiophen-3-yl)methanone for (4-morpholinophenyl) (thiazol-4-yl)methanone.

Step E: methyl 5-(1,1-dimethylethylsulfinamido)-5-(4-morpholinophenyl)-3-oxo-5-(thiazol-4-yl)pentanoate was prepared according to the Method 1, Step D substituting (Z)—N-((6-bromopyridin-2-yl)(thiophen-3-yl) methylene)-2-methylpropane-2-sulfinamide for (Z)-2-methyl-N-((4-morpholinophenyl) (thiazol-4-yl)methylene)propane-2-sulfinamide.

Step F: methyl 5-amino-5-(4-morpholinophenyl)-3-oxo-5-(thiazol-4-yl) pentanoate was prepared according to the Method 1, Step E substituting methyl 5-amino-5-(6-bromopyridin-2-yl)-3-oxo-5-(thiophen-3-yl)pentanoate for methyl 5-(1,1-dimethylethylsulfinamido)-5-(4-morpholinophenyl)-3 oxo-5-(thiazol-4-yl)pentanoate.

Step G: 4-hydroxy-6-(4-morpholinophenyl)-6-(thiazol-4-yl)-5,6-dihydropyridin-2(1H)-one was prepared in 18% yield over three steps according to the Method 1, Step F substituting methyl 5-amino-5-(6-bromopyridin-2-yl)-3-oxo-5-(thiophen-3-yl)pentanoate for methyl 5-amino-5-(4-morpholinophenyl)-3-oxo-5-(thiazol-4-yl)pentanoate.

Step H: 3-((2-chlorophenyl)thio)-6-(4-morpholinophenyl)-6-(thiazol-4-yl) piperidine-2,4-dione was prepared in 3% yield according to the Method 1, Step G substituting 6′-bromo-5-((2-chlorophenyl)thio)-4-hydroxy-2-(thiophen-3-yl)-2,3-dihydro-[2,2′-bipyridin]-6(1H)-one for 4-hydroxy-6-(4-morpholinophenyl)-6-(thiazol-4-yl)-5,6-dihydropyridin-2(1H)-one.

### Example 43

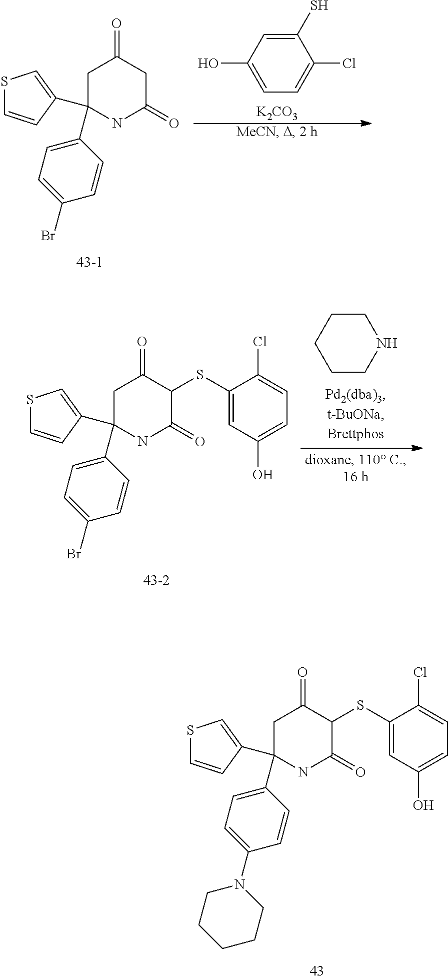

Step A: 6-(4-bromophenyl)-3-((2-chloro-5-hydroxyphenyl)thio)-6-(thiophen-3-yl) piperidine-2,4-dione was prepared in 68% yield according to the method 7 Step B substituting 1,2-bis(2-chlorophenyl)disulfane for 4-chloro-3-mercaptophenol.

Step B: In a solution of 6-(4-bromophenyl)-3-((2-chloro-5-hydroxyphenyl)thio)-6-(thiophen-3-yl)piperidine-2,4-dione (200 mg, 0.4 mmol) in dioxane (4 mL) was added piperidine (136 mg, 1.6 mmol), Brettphos (20 mg, 0.04 mmol), Pd₂(dba)₃ (18 mg, 0.02 mmol) and t-BuONa (154 mg, 1.6 mmol). The solution was stirred for 8 h at 110° C. under nitrogen atmosphere. The solvent was removed under vacuum and the residue was purified by Prep-HPLC (FA) to afford compound 43 (12 mg, 6%) as white solid.

The following compounds were prepared as indicated in the table, unless indicated otherwise, these compounds were prepared according to methods described herein.

| | | | IUPAC name/ | Characterization data |
|---|---|---|---|---|
| No. | ST* | Compound | synthesis | (NMR or MS) |
| 44 | MD | 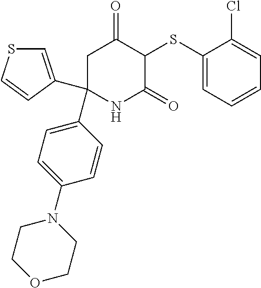 [SMILES: O=C1CC(c2ccc(N3CCOCC3)cc2)(c2ccsc2)NC(=O)C1Sc1ccccc1Cl] | 3-(2-chlorophenyl) sulfanyl-6-(4- morpholinophenyl)- 6-(3-thienyl) piperidine-2,4-dione was prepared in 10% yield according to the Example 7, Step H substituting 2-methylmorpholine for morpholine. | ¹H NMR (400 MHz, CD₃OD) δ 7.49 (dd,J = 7.6, 7.6 Hz, 1H), 7.43 (dd, J = 5.2, 5.2 Hz, 2H), 7.37 (d, J = 2.8 Hz, 1H), 7.35 (d, J = 2.8 Hz, 1H), 7.27 (d, J = 2.8 Hz, 1H), 7.21 (dd, J = 5.2, 5.2 Hz, 2H), 7.14 (d, J = 7.6 Hz, 1H), 6.94 (d, J = 7.6 Hz, 1H), 5.99 (d, J = 8.0 Hz, 1H), 3.86 (t, J = 3.6 Hz, 4H), 3.45 (d, J = 16.0 Hz, 1H), 3.31 (d, J = 16.0 Hz, 1H), 3.25 (t, J = 3.6 Hz, 4H). |
| 45 | MD | 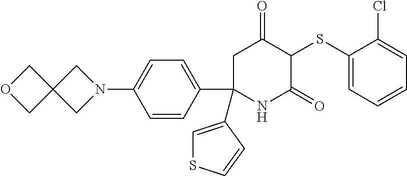 [SMILES: O=C1CC(c2ccc(N3CC4(COC4)C3)cc2)(c2ccsc2)NC(=O)C1Sc1ccccc1Cl] | 3-(2-chlorophenyl) sulfanyl-6-[4-(2-oxa- 6-azaspiro[3.3]heptan- 6-yl)phenyl]-6-(3- thienyl)piperidine- 2,4-dione was prepared in 26% yield according to the Example 7, Step H substituting 2-methyl- morpholine for 2-oxa- 6-azaspiro[3.3]heptane. | ¹H NMR (400 MHz, CD₃OD) δ 7.43 (dd, J = 5.2, 3.2 Hz, 1H), 7.29 (d, J = 3.2 Hz, 1H), 7.26 (d, J = 2.4 Hz, 1H), 7.25-7.24 (m, 3H), 7.06 (d, J = 2.4 Hz, 1H), 6.83 (dd, J = 5.2, 2.4 Hz, 1H), 6.66 (dd, J = 5.2, 2.4 Hz, 1H), 6.35 (d, J = 8.0 Hz, 2H), 6.02 (dd, J = 8.0 Hz, 1.2 Hz, 1H), 4.68 (s, 4H), 3.92 (s, 4H), 3.01 (s, 2H). |
| 46 | MD | 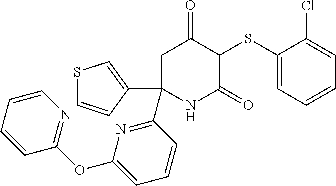 [SMILES: O=C1CC(c2ccsc2)(c2cccc(Oc3ccccn3)n2)NC(=O)C1Sc1ccccc1Cl] | 3-(2-chlorophenyl) sulfanyl-6-[6-(2- pyridyloxy)-2-pyridyl]- 6-(3-thienyl)piperidine- 2,4-dione was prepared in 0.5% yield according to Example 2, Step A substituting propan- 2-ol for pyridin-2-ol. | ¹H NMR (400 MHz, (CD₃)₂SO) δ 8.14 (m, 1H), 7.92 (m, 2H), 7.74 (d, J = 8.0 Hz, 1H), 7.54 (m, 2H), 7.39 (m, 1H), 7.26 (m, 2H), 6.90 (m, 1H), 6.56 (m, 1H), 6.52 (m, 1H), 6.40 (m, 1H), 5.78 (m, 1H), 3.79 (m, 1H), 3.33 (m, 1H). |
| 47 | MD | 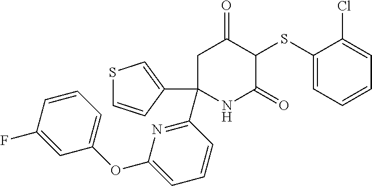 [SMILES: O=C1CC(c2ccsc2)(c2cccc(Oc3cccc(F)c3)n2)NC(=O)C1Sc1ccccc1Cl] | 3-(2-chlorophenyl) sulfanyl-6-[6-(3- fluorophenoxy)-2-pyridyl]- 6-(3-thienyl)piperidine- 2,4-dione was prepared in 8% yield according to the Example 3, Step A substituting 2-Chloro- 4-fluoro-phenol for 3-fluorophenol. | ¹H NMR (400 MHz, CD₃OD) δ 7.92 (dd, J = 7.6, 7.6 Hz, 1H), 7.44-7.41 (m, 3H), 7.38 (d, J = 2.8 Hz, 1H), 7.25 (d, J = 2.8 Hz, 1H), 7.23-7.33 (m, 2H), 7.07-7.05 (m, 2H), 7.03-6.95 (m, 3H), 6.06 (dd, J = 8.0, 1.2 Hz, 1H), 3.68 (d, J = 16.0 Hz, 1H), 3.34 (d, J = 16.0 Hz, 1H). |
| 48 | MD | 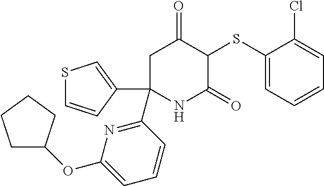 [SMILES: O=C1CC(c2ccsc2)(c2cccc(OC3CCCC3)n2)NC(=O)C1Sc1ccccc1Cl] | 3-((2-chlorophenyi)llii o)-6-(6-(cyclopentylox y)pyridin-2-yl)-6-(thio phen-3-yl)piperidine-2 .4-dione was prepared in 31% yield according to the Example 2, Step A substituting propan-2-ol for cyclopentanol | ¹H NMR (400 MHz, CD₃OD) δ 7.77 (dd, J = 8.0, 8.0 Hz, 1H), 7.47 (dd, J = 5.2, 3.2 Hz, 1H), 7.29-7.22 (m, 3H), 7.17 (dd, J = 5.2, 5.2 Hz, 1H), 6.96 (dd, J = 7.6, 7.6 Hz, 1H), 6.83-6.74 (m, 2H), 5.98 (dd, J = 8.0, 4.0 Hz, 1H), 5.64-5.62 (m, 1H), 4.05-3.79 (m, 5H), 3.50 (dd, J = 16.4, 1.6 Hz, 1H), 2.34-2.30 (m, 1H), 2.25- 2.03 (m, 1H). |
| 49 | MD | 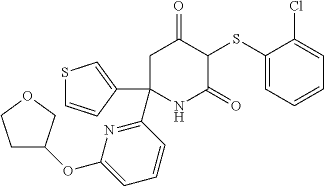 [SMILES: O=C1CC(c2ccsc2)(c2cccc(OC3CCOC3)n2)NC(=O)C1Sc1ccccc1Cl] | 3-((2-chlorophenyl)thio)- 6-(6-((tetrahydrofuran-3- yl)oxy)pyridin-2- yl)-6-(thiophen-3-yl) piperidine-2,4-dione was prepared in 29% yield according to the Example 2, Step A substituting propan-2-ol for tetrahydrofuran-3-ol | ¹H NMR (400 MHz, CD₃OD) δ 7.77 (dd, J = 8.0. 8.0 Hz, 1H), 7.47 (dd, J = 3.2, 1.6 Hz, 1H), 7.29- 7.15 (m, 3H), 7.09 (dd, J = 8.0, 8.0 Hz, 1H), 6.96 (dd, J = 7.6, 7.6 Hz, 1H), 6.83-6.74 (m, 2H), 5.99-5.96 (m,1H), 5.64-5.62 (m, 1H), 4.05-3.79 (m, 5H), 3.49 (dd, J = 16.4, 1.6 Hz, 2H), 2.34-2.30 (m, 1H), 2.25-2.03 (m, 1H). |
| 50 | MD | 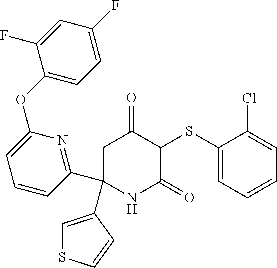 [SMILES: O=C1CC(c2ccsc2)(c2cccc(Oc3ccc(F)cc3F)n2)NC(=O)C1Sc1ccccc1Cl] | 3-(2-chlorophenyl) sulfanyl-6-[6-(2,4-difluoro phenoxy)-2-pyridyl]-6- (3-thienyl)piperidine- 2,4-dione was prepared in 1% yield according to the Example 3, Step A substituting 2-Chloro-4-fluoro- phenol for 2,4-difluorophenol. | ¹H NMR (400 MHz, CD₃OD) δ 7.89 (dd, J = 7.6, 7.6 Hz, 1H), 7.38-7.33 (m, 2H), 7.33-7.23 (m, 2H), 7.21-7.12 (m, 3H), 7.11- 6.92 (m, 3H), 6.78 (dd, J = 8.0, 8.0 Hz, 1H), 5% (d, J = 8.0 Hz, 1H), 3.51 (d, J = 16.0 Hz, 1H), 3.24 (d, J = 16.0 Hz, 1H). |
| 51 | MD | 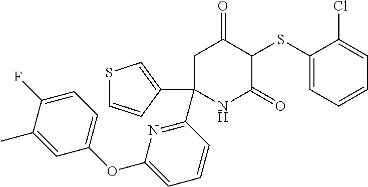 [SMILES: Cc1cc(Oc2cccc(C3(c4ccsc4)CC(=O)C(Sc4ccccc4Cl)C(=O)N3)n2)ccc1F] | 5-((2-chlorophenyl)thio)- 6′-(4-fluoro-3-methyl- phenoxy)-4-hydroxy- 2-(thiophen-3-yl)-2,3- dihydro-[2,2′-bipyridin]- 6(1H)-one was prepared in 0.3% yield according to the Example 3, Step A substituting 2-Chloro-4- fluoro-phenol for 4- fluoro-3-methylphenol | ¹H NMR (400 MHz, (CD₃)₂SO) δ 8.32 (s, 1H), 7.90 (dd, J = 8.0, 8.0 Hz, 1H), 7.47 (dd, J = 4.9, 3.1 Hz, 1H), 7.39 (d, J = 7.5 Hz, 1H), 7.35 (d, J = 7.7 Hz, 1H), 7.23 (dd, J = 1.2, 1.2 Hz, 1H), 7.13 (dd, J = 9.2, 8.8 Hz, 1H), 7.00- 7.02 (m, 1H), 6.91-6.97 (m, 4H), 6.75 (t, J = 7.4 Hz, 1H), 5.91 (d, J = 7.9 Hz, 1H), 3.53 (d, J = 16.3 Hz, 2H), 3.22 (d, J = 16.3 Hz, 1H), 2.17 (s, 3H). |
| 52 | MD | 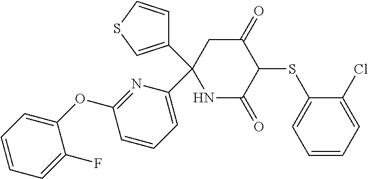 [SMILES: O=C1CC(c2ccsc2)(c2cccc(Oc3ccccc3F)n2)NC(=O)C1Sc1ccccc1Cl] | 3-(2-chlorophenyl)sulfanyl- 6-[6-(2-fluorophe noxy)-2-pyridyl]-6-(3- thienyl)piperidine-2,4- dione was prepared in 8.2% yield according to the Example 3, Step A substituting 2-Chloro-4-fluoro- phenol for 2-fluorophenol | ¹H NMR (400 MHz, CD₃OD) δ 7.68 (dd, J = 8.0, 8.0 Hz, 1H), 7.40 (dd, J = 4.8, 2.0 Hz, 1H), 7.36-7.24 (m, 6H), 7.23-7.17 (m, 1H), 7.02 (d, J = 8.0 Hz, 1H), 6.98-6.75 (m, 3H), 5.91 (dd, J = 8.0, 1.6 Hz, 1H), 3.42 (d, J = 16.0 Hz, 1H), 3.17 (d, J = 16.0 Hz, 1H). |
| 53 | MD | 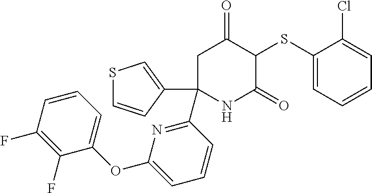 [SMILES: O=C1CC(c2ccsc2)(c2cccc(Oc3cccc(F)c3F)n2)NC(=O)C1Sc1ccccc1Cl] | 3-(2-chlorophenyl)sulfanyl- 6-[6-(2,3-difluoro- phenoxy)-2-pyridyl]-6- (3-thienyl)piperidine- 2,4-dione was prepared in 2% yield according to the Example 2. Step A substituting propan-2-ol for 2,3-difluorophenol. | ¹H NMR (400 MHz, CD₃OD) δ 7.92 (dd, J = 8.0, 8.0 Hz, 1H), 7.41-7.35 (m, 2H), 7.23-7.15 (m, 4H), 7.09 (d, J = 8.3 Hz, 1H), 7.00 (dd, J = 5.2, 1.2 Hz, 1H), 6.96-6.89 (m, 1H), 6.83-6.76 (m, 1H), 6.08 (d, J = 8.5 Hz, 1H), 3.46 (d, J = 16.0 Hz, 1H), 3.26 (d, J = 16.0 Hz, 1H). |
| 54 | MD | 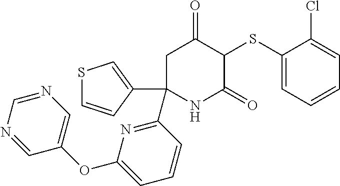 [SMILES: O=C1CC(c2ccsc2)(c2cccc(Oc3cncnc3)n2)NC(=O)C1Sc1ccccc1Cl] | 3-(2-chlorophenyl)sulfanyl- 6-(6-pyrimidin-5- yloxy-2-pyridyl)-6-(3- thienyl)piperidine-2,4- dione was prepared in 6% yield according to Example 2, Step A substituting propan-2-ol for pyrimidin-5-ol. | ¹H NMR (400 MHz, (CD₃)₂SO) 5 9.08 (s, 1H), 8.73 (s, 2H), 8.45 (s, 1H), 8.13 (dd, J = 8.0, 8.0 Hz, 1H), 7.50 (m, 2H), 7.31 (dd, J = 6.8, 4.8 Hz, 1H), 7.24 (m, 2H), 7.02 (m, 2H), 6.83 (dd, J = 8.8, 4.4 Hz, 1H), 5.93 (d, J = 8.0 Hz, 1H), 3.38 (s, 1H), 3.28 (s, 1H). |
| 55 | MD | 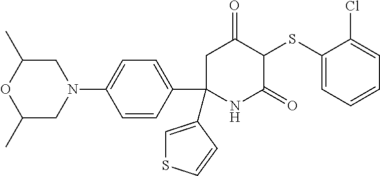 [SMILES: CC1CN(c2ccc(C3(c4ccsc4)CC(=O)C(Sc4ccccc4Cl)C(=O)N3)cc2)CC(C)O1] | 3-(2-chlorophenyl)sulfanyl- 6-[4-(2,6-dimethyl- morpholin-4-yl)phenyl]- 6-(3-thienyl)piperidine- 2,4-dione was prepared in 24% yield according to the Example 7, Step H substituting 2- methylmorpholine for 2,6-dimethylmorpholine. | ¹H NMR (400 MHz, CD₃OD) δ 7.48 (dd, J = 5.2, 3.2 Hz, 1H), 7.34-7.32 (m, 2H), 7.31 (d, J = 3.2 Hz, 1H), 7.28 (d, J = 2.4 Hz, 1H), 7.27 (d, J = 2.4 Hz, 1H), 6.99-6.97 (m, 2H), 6.90 (dd, J = 5.2, 2.4 Hz, 1H), 6.73 (dd, J = 5.2, 2.4 Hz, 1H), 6.06 (dd, J = 8.0, 1.2 Hz, 1H), 3.85-3.78 (m, 2H), 3.36 (d, J = 16.0 Hz, 2H), 3.32 (t, J = 5.2 Hz, 2H), 3.38 (t, J = 5.2 Hz, 2H), 1.38 (s, 6H). |
| 56 | MD | 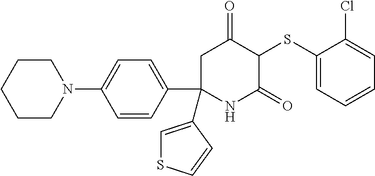 [SMILES: O=C1CC(c2ccc(N3CCCCC3)cc2)(c2ccsc2)NC(=O)C1Sc1ccccc1Cl] | 3-(2-chlorophenyl)sulfanyl- 6-[4-(1-piperidyl)phenyl]- 6-(3-thienyl)piperidine- 2,4-dione was prepared in 12% yield according to the Example 7, Step H substituting 2- methylmorpholine for piperidine. | ¹H NMR (400 MHz, CD₃OD) δ 7.47 (dd, J = 5.2, 3.2 Hz, 1H), 7.33- 7.31 (m, 2H), 7.27 (d, J = 3.2 Hz, 1H), 7.19-7.17 (m, 2H), 7.16- 7.14 (m, 2H), 6.90 (dd, J = 5.2, 2.8 Hz, 1H), 6.76 (dd, J = 5.2, 2.8 Hz, 1H), 6.08 (dd, J = 8.0, 1.2 Hz, 1H), 3.35 (d, J = 16.0 Hz, 2H), 3.20 (t, J = 5.2 Hz, 4H), 1.77- 1.72 (m, 4H), 1.66-1.60 (m, 4H). |
| 57 | MD | 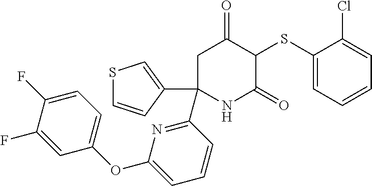 [SMILES: O=C1CC(c2ccsc2)(c2cccc(Oc3ccc(F)c(F)c3)n2)NC(=O)C1Sc1ccccc1Cl] | 3-(2-chlorophenyl)sulfanyl- 6-[6-(3,4-difluoro- phenoxy)-2-pyridyl]-6- (3-thienyl)piperidine-2,4- dione was prepared in 7% yield according to the Example 3, Step A substituting 2-chloro-4- fluoro-phenol for 3,4- difluorophenol. | ¹H NMR (400 MHz, CD₃OD) δ 7.88 (dd, J = 7.6, 7.6 Hz, 1H), 7.41- 7.37 (m, 2H), 7.35-7.21 (m, 3H), 7.18-6.97 (m, 4H), 6.95-6.78 (m, 2H), 5.98 (d, J = 8.0 Hz, 1H), 3.61 (d, J = 16.0 Hz, 1H), 3.34 (d, J = 16.0 Hz, 1H) |
| 58 | MD | 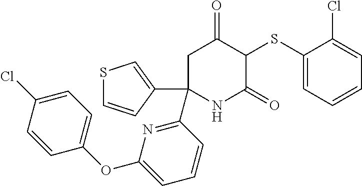 [SMILES: O=C1CC(c2ccsc2)(c2cccc(Oc3ccc(Cl)cc3)n2)NC(=O)C1Sc1ccccc1Cl] | 6′-(4-chlorophenoxy)-5- ((2-chlorophenyl)thio)-4- hydroxy-2-(thiophen-3- yl)-2,3-dihydro-[2,2′- bipyridin]-6(1H)-one was prepared in 15.6% yield according to the Example 2, Step A substituting propan-2-ol for 4- chlorophenol. | ¹H NMR (400 MHz, (CD₃)₂SO) δ 11.50 (s, 1H), 8.50 (s, 1H), 7.98 (dd, J = 7.8, 7.8 Hz, 1H),7.51 (dd, J = 5.0, 3.0 Hz, 1H), 7.40-7.48 (m, 3H), 7.29-7.35 (m, 1H), 7.27 (dd, J = 2.9, 1.3 Hz, 1H), 7.09- 7.15 (m, 2H), 6.96-7.06 (m, 3H), 6.77-6.83 (m, 1H), 5.93 (dd, J = 7.9, 1.3 Hz, 1H), 3.59 (d, J = 16.5 Hz, 1H), 3.25 (d, J = 16.5 Hz, 1H). |
| 59 | MD | 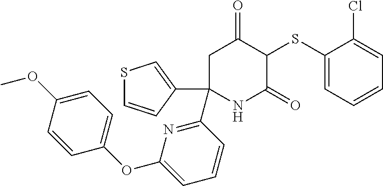 [SMILES: COc1ccc(Oc2cccc(C3(c4ccsc4)CC(=O)C(Sc4ccccc4Cl)C(=O)N3)n2)cc1] | 3-(2-chlorophenyl)sulfanyl- 6-[6-(4-methoxyphenoxy)- 2-pyridyl]-6-(3-thienyl) piperidine-2,4-dione was prepared in 41% yield according to the Example 2, Step A substituting propan-2-ol for 4- methoxyphenol. | ¹H NMR (400 MHz, CD₃OD) δ 7.80 (dd, J = 8.0, 8.0 Hz, 1H), 7.36 (dd, J = 4.8, 2.8 Hz, 1H), 7.28 (d, J = 8.4 Hz, 1H), 7.26 (dd, J = 5.2, 1.2 Hz, 1H), 7.15 (dd, J = 7.6, 1.2 Hz, 1H), 7.07-6.96 (m, 5H), 6.88- 6.81 (m, 2H), 6.78-6.73 (m, 1H), 6.17 (dd, J = 8.0, 1.2 Hz, 1H), 3.83 (s, 3H), 3.42 (d, J = 16.0 Hz, 1H), 3.30 (d, J =16.0 Hz, 1H). |
| 60 | MD | 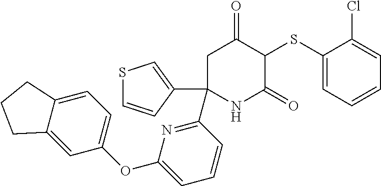 [SMILES: O=C1CC(c2ccsc2)(c2cccc(Oc3ccc4c(c3)CCC4)n2)NC(=O)C1Sc1ccccc1Cl] | 5-((2-chlorophenyl)thio)- 6′-((2,3-dihydro-1H- inden-5-yl)oxy)-4- hydroxy-2-(thiophen-3-yl)- 2,3-dihydro-[2,2′- bipyridin]-6(1H)-one was prepared in 7% yield according to the Example 2, Step A substituting propan-2-ol for 2,3- dihydro-1H-inden-5-ol. | ¹H NMR (400 MHz, (CD₃)₂SO) δ 8.40-8.29 (m, 1H), 7.88 (d, J = 8.0, 8.0 Hz, 1H), 7.48-7.42 (m, 1H), 7.37 (d, J = 7.6 Hz, 1H), 7.26-7.25 (m, 2H), 7.20 (d, J = 5.2 Hz, 1H), 7.04 (d, J = 6.4 Hz, 1H), 6.98-6.92 (m, 1H), 6.88- 6.83 (m, 3H), 6.80-6.75 (m, 1H), 5.92 (d, J = 8.0 Hz, 1H), 3.65- 3.58 (m, 1H), 3.42-3.38 (m, 1H), 2.88-2.78 (m, 4H), 2.05-1.99 (m, 2H). |
| 61 | MD | 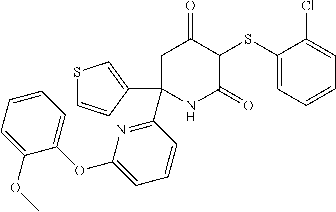 [SMILES: COc1ccccc1Oc1cccc(C2(c3ccsc3)CC(=O)C(Sc3ccccc3Cl)C(=O)N2)n1] | 3-(2-chlorophenyl)sulfanyl- 6-[6-(2-methoxyphenoxy)- 2-pyridyl]-6-(3-thienyl) piperidine-2,4-dione was prepared in 24% yield according to the Example 2, Step A substituting propan-2-ol for 2- methoxyphenol. | ¹H NMR (400 MHz, CD₃OD) δ 7.82 (dd, J = 8.4, 8.4 Hz, 1H), 7.37 (dd, J = 5.6, 3.2 Hz, 1H), 7.28-7.24 (m, 2H), 7.22 (dd, J = 8.0, 1.6 Hz, 1H), 7.17 (dd, J = 2.8, 1.6 Hz, 1H), 7.12 (dd, J = 4.4, 1.6 Hz, 1H), 7.10 (dd, J = 5.2, 1.6 Hz, 1H), 7.02-6.98 (m, 2H), 6.97- 6.90 (m, 2H), 6.84-6.76 (m, 1H), 6.08 (dd, J = 6.4, 1.6 Hz, 1H), 3.61 (s, 3H), 3.52 (d, J = 16.4 Hz, 1H), 3.26 (d, J = 16.0 Hz, 1H). |
| 62 | MD | 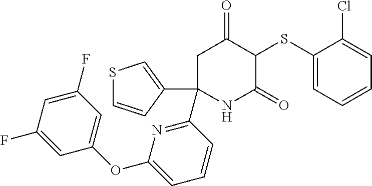 [SMILES: O=C1CC(c2ccsc2)(c2cccc(Oc3cc(F)cc(F)c3)n2)NC(=O)C1Sc1ccccc1Cl] | 3-(2-chlorophenyl)sulfanyl- 6-[6-(3,5-difluoro- phenoxy)-2-pyridyl]-6- (3-thienyl)piperidine-2,4- dione was prepared in 2% yield according to the Example 3, Step A substituting 2-chloro-4- fluoro-phenol for 3,5- difluorophenol. | ¹H NMR (400 MHz, CD₃OD) δ 7.92 (dd, J = 7.5, 7.5 Hz, 1H), 7.40 (d, J = 3.2 Hz, 2H), 7.22 (d, J = 2.8 Hz, 2H), 7.06 (d, J = 2.4 Hz, 2H), 6.95 (dd, J = 7.6, 7.6 Hz, 1H), 6.81-6.03 (m, 4H), 6.01 (d, J = 8.0 Hz, 1H), 3.65 (d, J = 16.0 Hz, 1H), 3.34 (d, J = 16.0 Hz, 1H) |
| 63 | MD | 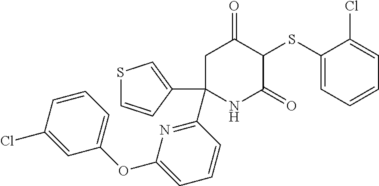 [SMILES: O=C1CC(c2ccsc2)(c2cccc(Oc3cccc(Cl)c3)n2)NC(=O)C1Sc1ccccc1Cl] | 6′-(3-chlorophenoxy)-5- ((2-chlorophenyl)thio)- 4-hydroxy-2-(thiophen-3- yl)-2,3-dihydro-[2,2′- bipyridin]-6(1H)-one was prepared in 13.9% yield according to the Example3 2, Step A substituting propan-2-ol for 3- chlorophenol. | ¹H NMR (400 MHz, (CD₃)₂SO) δ 11.49 (s, 1H), 8.50 (s, 1H), 7.99 (dd, J = 8.0, 8.0 Hz, 1H), 7.51 (dd, J = 5.1, 2.9 Hz, 1H), 7.47 (d, J = 7.5 Hz, 1H), 7.39-7.45 (m, 1H), 7.26-7.34 (m, 3H), 7.20 (dd, J = 2.1, 2.1 Hz, 1H), 7.02-7.09 (m, 3H), 6.94-7.02 (m, 2H), 6.81 (dd, J = 7.2, 7.2 Hz, 1H), 5.92-5.97 (m, 1H), 3.60 (d, J = 16.5 Hz, 1H), 3.26 (d, J = 16.5 Hz, 1H). |
| 64 | MD | 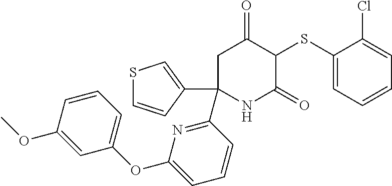 [SMILES: COc1cccc(Oc2cccc(C3(c4ccsc4)CC(=O)C(Sc4ccccc4Cl)C(=O)N3)n2)c1] | 3-(2-chlorophenyl)sulfanyl- 6-[6-(3-methoxyphenoxy)- 2-pyridyl]-6-(3-thienyl) piperidine-2,4-dione was prepared in 9% yield according to the Example 2, Step A substituting propan-2-ol for 3- methoxyphenol. | ¹H NMR (400 MHz, CD₃OD) δ 7.85 (dd, J = 7.8, 7.8 Hz, 1H), 7.39 (dd, J = 5.2, 2.8 Hz, 1H), 7.34 (d, J = 7.6 Hz, 1H), 7.32-7.28 (m, 1H), 7.26 (dd, J = 2.8, 1.2 Hz, 1H), 7.19 (dd, J = 7.6, 1.2 Hz, 1H), 7.08 (dd, J = 4.8, 1.2 Hz, 1H), 6.93 (d, J = 7.8 Hz, 1H), 6.92- 6.88 (m, 1H), 6.81-6.77 (m, 2H), 6.90-6.66 (m, 2H), 6.13 (dd, J = 8.0, 1.6 Hz, 1H), 3.76 (s, 3H), 3.54 (d, J = 16.0 Hz, 1H), 3.34 (d, J = 16.0 Hz, 1H). |
| 65 | MD | 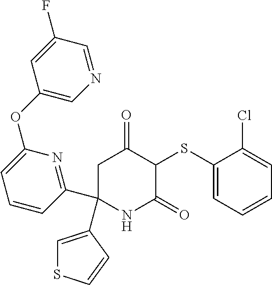 [SMILES: O=C1CC(c2ccsc2)(c2cccc(Oc3cncc(F)c3)n2)NC(=O)C1Sc1ccccc1Cl] | 3-(2-chlorophenyl)sulfanyl- 6-[6-[(5-fluoro-3- pyridyl)oxy]-2-pyridyl]- 6-(3-thienyl)piperidine- 2,4-dione was prepared in 6.9% yield according to the Example 3, Step A substituting 2-Chloro-4- fluoro-phenol for 5- fluoropyridin-3-ol | ¹H NMR (400 MHz, (CD₃)₂SO) δ 8.45 (d, J= 2.8 Hz, 1H), 8.32 (s, 1H), 7.80 (dd, J = 7.6, 7.6 Hz, 1H), 7.55 (dd, J = 7.6, 7.6 Hz, 1H), 7.44 (dd, J = 7.2, 7.2 Hz, 2H), 7.25-7.21 (m, 2H), 7.10 (d, J = 8.0 Hz, 1H), 7.00 (s, 1H), 6.97-693 (m, 1H), 6.76 (dd, J = 7.6, 7.6 Hz, 1H), 5.91 (d, J = 7.6 Hz, 1H)..93 |
| 66 | MD | 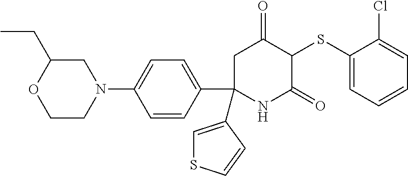 [SMILES: CCC1CN(c2ccc(C3(c4ccsc4)CC(=O)C(Sc4ccccc4Cl)C(=O)N3)cc2)CCO1] | 3-(2-chlorophenyl)sulfanyl- 6-[4-(2-ethylmorpholin- 4-yl)phenyl]-6-(3-thienyl) piperidine-2,4-dione was prepared in 8% yield according to the Example 7, Step H substituting 2- methylmorpholine for 2- ethylmorpholine. | ¹H NMR (400 MHz, CD₃OD) δ 7.43 (dd, J = 5.2, 3.2 Hz, 1H), 7.36- 7.27 (m, 2H), 7.12 (d, J = 3.2 Hz, 1H), 7.10 (d, J = 2.4 Hz, 1H), 6.98 (d, J = 2.4 Hz, 1H), 6.96-6.81 (m, 2H), 6.78 (dd, J = 5.2, 2.4 Hz, 1H), 6.71 (dd, J = 5.2, 2.4 Hz, 1H), 6.17 (dd, J = 8.0, 1.2 Hz, 1H), 4.03-3.99 (m, 1H), 3.79 (d, J = 16.0 Hz, 1H), 3.56 (d, J = 16.0 Hz, 1H), 3.54 (t, J = 4.4 Hz, 2H), 3.18 (d, J = 4.8 Hz, 2H), 2.80 (dd, J = 4.4, 1.6 Hz, 1H), |
| | | | | 2.77 (dd, J = 4.4, 1.6 Hz, 1H), |
| | | | | 1.96-1.62 (m, 2H), 1.01 (t, J = 3.6 |
| | | | | Hz, 3H). |
| 67 | MD | 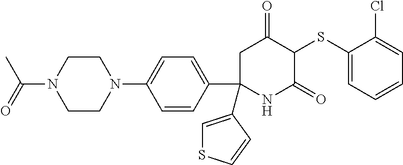 [SMILES: CC(=O)N1CCN(c2ccc(C3(c4ccsc4)CC(=O)C(Sc4ccccc4Cl)C(=O)N3)cc2)CC1] | 6-[4-(4-acetylpiperazin-1- yl)phenyl]-3-(2- chlorophenyl)sulfanyl- 6-(3-thienyl)piperidine- 2,4-dione was prepared in 2% yield according to the Example 7, Step H substituting 2-methyl- morpholine for 1- (piperazin-1-yl)ethanone. | ¹H NMR (400 MHz, CD₃OD) δ 7.48 (dd, J = 2.8, 2.8 Hz, 1H), 7.37- 7.36 (m, 2H), 7.33 (d, J = 2.8 Hz, 1H), 7.28 (d, J = 2.4 Hz, 1H), 7.19 (d, J = 2.4 Hz, 1H), 7.16 (d, J = 2.8 Hz, 1H), 7.04-7.01 (1H, 2H), 6.90 (dd, J = 5.2, 2.4 Hz, 1H), 6.73 (dd, J = 5.2, 2.4 Hz, 1H), 6.07 (dd, J = 8.0, 1.2 Hz, 1H), 3.77-3.70 (1H, 4H), 3.36 (d, J = 16.0 Hz, 2H), 3.33-3.19 (m, 4H), 2.17 (s, 3H). |
| 68 | MD | 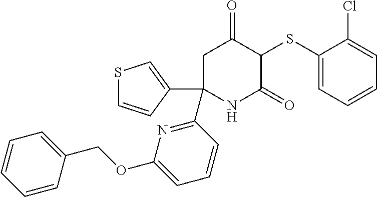 [SMILES: O=C1CC(c2ccsc2)(c2cccc(OCc3ccccc3)n2)NC(=O)C1Sc1ccccc1Cl] | 6′-(benzyloxy)-5-((2- chlorophenyl)thio)-4- hydroxy-2-(thiophen-3- yl)-2,3-dihydro-[2,2′- bipyridin]-6(1H)-one was prepared in 2.1% yield according to the Example 2, Step A substituting propan-2-ol for phenylmethanol. | ¹H NMR (400 MHz, CD₃OD) δ 7.63 (dd, J = 8.0, 8.0 Hz, 1H), 7.37 (d, J = 6.8 Hz, 2H), 7.24-7.32 (m, 5H), 7.15 (d, J = 9.0 Hz, 2H), 7.10 (s, 1H), 7.01 (d, J = 1.5 Hz, 1H), 6.95 (d, J = 4.0 Hz, 1H), 6.90 (dd, J = 8.0, 8.0 Hz, 1H), 6.71-6.78 (m, 2H), 6.04 (d, J = 8.3 Hz, 1H), 5.32-5.43 (m, 2H), 3.78 (d, J = 16.8 Hz, 1H), 3.40 (d, J = 16.6 Hz, 1H). |
| 69 | MD | 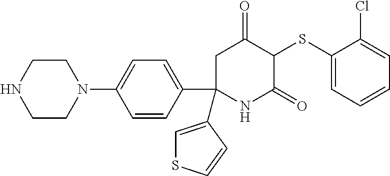 [SMILES: O=C1CC(c2ccc(N3CCNCC3)cc2)(c2ccsc2)NC(=O)C1Sc1ccccc1Cl] | 3-(2-chlorophenyl)sulfanyl- 6-(4-piperazin-1- ylphenyl)-6-(3-thienyl) piperidine-2,4-dione was prepared in 2% yield according to the Example 7, Step H substituting 2- methylmorpholine for piperazine. | ¹H NMR (400 MHz, CD₃OD) δ 7.42 (dd, J = 7.6, 7.6 Hz, 1H), 7.34 (dd, J = 5.2, 5.2 Hz, 2H), 7.26 (d, J = 3.2 Hz, 1H), 7.12- 7.11 (m, 2H), 6.97 (dd, J = 4.8, 4.8 Hz, 2H), 6.81 (d, J = 7.6 Hz, 1H), 6.77 (d, J = 7.6 Hz, 1H), 6.17 (dd, J = 8.0, 1.2 Hz, 1H), 3.19-3.16 (m, 6H), 3.01-2.99 (m, 4H). |
| 70 | MD | 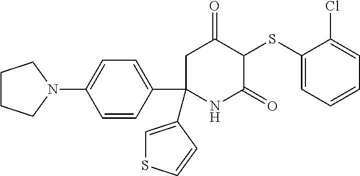 [SMILES: O=C1CC(c2ccc(N3CCCC3)cc2)(c2ccsc2)NC(=O)C1Sc1ccccc1Cl] | 3-(2-chlorophenyl)sulfanyl- 6-(4-pyrrolidin-1- ylphenyl)-6-(3-thienyl) piperidine-2,4-dione was prepared in 3% yield according to the Example 7, Step H substituting 2- methylmorpholine for pyrrolidine. | ¹H NMR (400 MHz, CD₃OD) δ 7.44 (dd, J = 4.8, 2.8 Hz, 1H), 7.23-7.20 (m, 3H), 7.15 (d, J = 3.2 Hz, 1H), 7.12 (d, J = 3.2 Hz, 1H), 6.85 (dd, J = 7.6, 1.6 Hz, 1H), 6.67 (d, J = 7.6 Hz, 1H), 6.62 (d, J = 1.2 Hz, 1H), 6.57 (d, J = 1.2 Hz, 1H), 6.02 (dd, J = 8.0, 1.2 Hz, 1H), 3.34 (d, J = 12.0 Hz, 2H), 3.30- 3.26 (m, 4H), 2.03-2.00 (m, 4H). |
| 71 | MD | 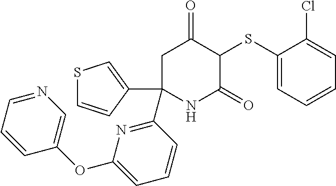 [SMILES: O=C1CC(c2ccsc2)(c2cccc(Oc3cccnc3)n2)NC(=O)C1Sc1ccccc1Cl] | 3-((2-chlorophenyl)thio)- 6-(6-(pyridin-3-yloxy) pyridin-2-yl)-6-(thiophen- 3-yl)piperidine-2,4- dione was prepared in 1% yield according to the Example 2, Step A substituting propan-2-ol for pyridin-3-ol. | ¹H NMR (400 MHz, CD₃OD) δ 8.73-8.72 (m, 1H), 8.69-8.56 (m, 1H), 8.27-8.22 (m, 1H), 8.02-7.96 (m, 2H), 7.42-7.36 (m, 2H), 7.19- 7.13 (m, 3H), 6.97 (dd, J = 5.2, 1.2 Hz, 1H), 6.90-6.85 (m, 1H), 6.78-6.70 (m, 1H), 5.90 (d, J = 8.0 Hz, 1H), 3.43 (d, J = 16.0 Hz, 1H), 3.36 (d, J = 16.0 Hz, 1H). |
| 72 | MD | 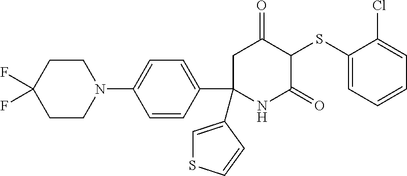 [SMILES: O=C1CC(c2ccc(N3CCC(F)(F)CC3)cc2)(c2ccsc2)NC(=O)C1Sc1ccccc1Cl] | 3-(2-chlorophenyl)sulfanyl- 6-[4-(4,4-difluoro-1- piperidyl)phenyl]-6-(3- thienyl)piperidine-2,4- dione was prepared in 7.4% yield according to the Example 4, Step a substituting cyclo- hexanamine for 4,4- difluoropiperidine | ¹H NMR (400 MHz, CD₃OD) δ 7.49 (dd, J = 5.2, 2.8 Hz, 1H), 7.31 (d, J = 9.2 Hz, 2H), 7.27 (dd, J = 3.2, 1.6 Hz, 1H), 7.20 (dd, J = 7.6, 0.8 Hz, 1H), 7.14 (dd, J = 5.2, 1.2 Hz, 1H), 7.03 (d, J = 8.8 Hz, 1H), 6.93-6.89 (m, 2H), 6.76-6.72 (m, 1H), 5.98 (dd, J = 8.0, 1.2 Hz, 1H), 3.44 (d, J = 1.2 Hz, 2H), 3.40 (t, J = 5.6 Hz, 4H), 2.12-2.02 (m, 4H). |
| 73 | MD | 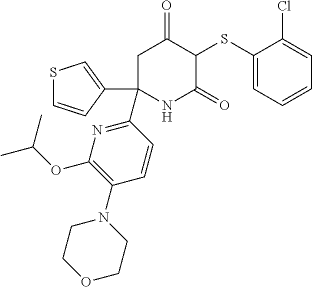 [SMILES: CC(C)Oc1nc(C2(c3ccsc3)CC(=O)C(Sc3ccccc3Cl)C(=O)N2)ccc1N1CCOCC1] | 3-((2-chlorophenyl)thio)- 6-(6-isopropoxy-5- morpholinopyridin-2-yl)- 6-(thiophen-3-yl) piperidine-2,4-dione was prepared in 25% yield according to the Example 2, Step A substituting 6′-bromo-5-((2-chloro- phenyl)thio)-4-hydroxy- 2-(thiophen-3-yl)-2,3- dihydro-[2,2′-bipyridin]- 6(1H)-onel for 6′-bromo- 5-((2-chlorophenyl)thio)- 4-hydroxy-5′-morpholino- 2-(thiophen-3-yl)-2,3- dihydro-[2,2′-bipyridin]- 6(1H)-one | ¹H NMR (400 MHz, CD₃OD) δ 7.44 (dd, J = 5.2, 3.2 Hz, 1H), 7.27-7.23 (m, 3H), 7.16 (dd, J = 4.8, 1.2 Hz, 1H), 7.09 (d, J = 7.6 Hz, 1H), 6.95 (dd, J = 8.0, 6.0 Hz, 1H), 6.78 (dd, J = 8.0, 1.6 Hz, 1H), 6.03 (dd, J = 7.6, 1.2 Hz, 1H), 5.46-5.43 (m, 1H), 3.90-3.86 (m, 5H), 3.45 (d, J = 16.0 Hz, 1H), 3.14 (d, J = 4.0 Hz, 4H), 1.36 (dd, J = 18.0, 6.4 Hz, 6H). |
| 74 | MD | 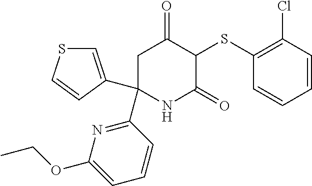 [SMILES: CCOc1cccc(C2(c3ccsc3)CC(=O)C(Sc3ccccc3Cl)C(=O)N2)n1] | 3-(2-chlorophenyl)sulfanyl- 6-(6-ethoxy-2-pyridyl)-6- (3-thienyl)piperidine-2,4- dione was prepared in 4.9% yield according to the Example 2, Step A substituting propan-2- ol for ethanol | ¹H NMR (400 MHz, CD₃OD) δ 7.72 (dd, J = 8.0, 8.0 Hz, 1H), 7.46-7.44 (m, 1H), 7.30-7.15 (1H, 4H), 6.98-6.94 (m, 1H), 6.79-6.75 (m, 2H), 6.02 (d, J = 7.2 Hz, 1H), 4.45-4.39 (m, 2H), 3.92 (d, J = 16.0 Hz, 1H), 3.48 (d, J = 16.0 Hz, 1H), 1.36 (t, J = 1.2 Hz, 3H). |
| 75 | MD | 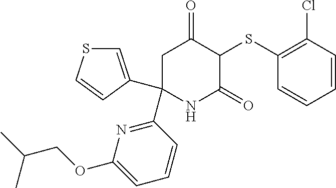 [SMILES: CC(C)COc1cccc(C2(c3ccsc3)CC(=O)C(Sc3ccccc3Cl)C(=O)N2)n1] | 3-(2-chlorophenyl)sulfanyl- 6-(6-isobutoxy-2-pyridyl)- 6-(3-thienyl)piperidine- 2,4-dione was prepared in 6.7% yield according to the Example 2, Step A substituting propan-2-ol for 2-methylpropan-1-ol | ¹H NMR (400 MHz, CD₃OD) δ 7.74 (dd, J = 7.6, 7.6 Hz, 1H), 7.46-7.44 (m, 1H), 7.30-7.14 (1H, 4H), 6.97-6.93 (m, 1H), 6.79-6.75 (m, 2H), 6.00 (d, J = 7.2 Hz, 1H), 4.18-4.10 (1H, 2H), 3.91 (d, J = 16.0 Hz, 1H), 3.47 (d, J = 16.0 Hz, 1H), 2.10-2.00 (m, 2H), 1.00 (t, J = 6.4 Hz, 6H). |
| 76 | MD | 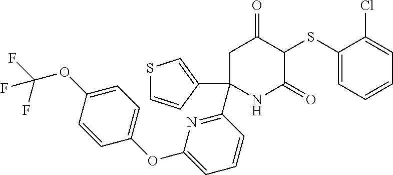 [SMILES: O=C1CC(c2ccsc2)(c2cccc(Oc3ccc(OC(F)(F)F)cc3)n2)NC(=O)C1Sc1ccccc1Cl] | 3-(2-chlorophenyl)sulfanyl- 6-(3-thienyl)-6-[6-[4- (trifluoromethoxy) phenoxy]-2-pyridyl] piperidine-2,4-dione was prepared in 3% yield according to the Example 3, Step A substituting 2- chloro-4-fluoro-phenol for 4-(trifluoromethoxy) phenol. | ¹H NMR (400 MHz, CD₃OD) δ 7.91 (dd, J = 8.0, 8.0 Hz, 1H), 7.40-7.36 (m, 2H), 7.30-7.23 (m, 3H), 7.18-7.13 (m, 3H), 7.05-6.94 (m, 3H), 6.81 (dd, J = 6.8, 6.8 Hz, 1H), 6.02 (d, J = 7.2 Hz, 1H), 3.63 (d, J = 16.0 Hz, 1H), 3.34 (d, J = 6.8 Hz, 1H) |
| 77 | SS | 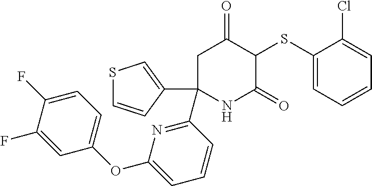 [SMILES: O=C1CC(c2ccsc2)(c2cccc(Oc3ccc(F)c(F)c3)n2)NC(=O)C1Sc1ccccc1Cl] | 3-(2-chlorophenyl)sulfanyl- 6-[6-(3,4-difluoro- phenoxy)-2-pyridyl]-6-(3- thienyl)piperidine-2,4- dione was prepared in 7% yield according to the Example 3, Step A substituting 2-chloro-4- fluoro-phenol for 3,4- difluorophenol. | ¹H NMR (400 MHz, CD₃OD) δ 7.89 (dd, J = 7.6, 7.6 Hz, 1H), 7.40 (d, J = 2.3 Hz, 1H), 7.38 (d, J = 2.5 Hz, 1H), 7.36-7.19 (m, 3H), 7.06-7.00 (m, 3H), 6.95 (d, J = 7.5 Hz, 1H), 6.87 (d, J = 8.0 Hz, 1H), 6.78 (d, J = 7.5 Hz, 1H), 6.01 (d, J = 8.0 Hz, 1H), 3.58 (d, J = 16.0 Hz, 1H), 3.49 (d, J = 16.0 Hz, 1H). |
| 78 | SS | 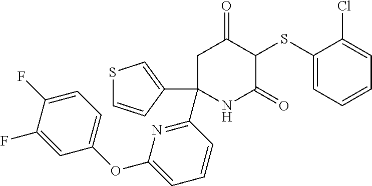 [SMILES: O=C1CC(c2ccsc2)(c2cccc(Oc3ccc(F)c(F)c3)n2)NC(=O)C1Sc1ccccc1Cl] | 3-(2-chlorophenyl)sulfanyl- 6-[6-(3,4-difluoro- phenoxy)-2-pyridyl]-6- (3-thienyl)piperidine- 2,4-dione was prepared in 7% yield according to the Example 3, Step A substituting 2-chloro- 4-fluoro-phenol for 3,4-difluorophenol. | ¹H NMR (400 MHz, CD₃OD) δ 7.88 (dd, J = 7.5, 7.5 Hz, 1H), 7.40 (d, J = 2.3 Hz, 1H), 7.33 (d, J = 2.4 Hz, 1H), 7.37-7.19 (m, 3H), 7.06-7.01 (m, 3H), 6.96 (d, J = 7.5 Hz, 1H), 6.89 (d, J = 8.0 Hz, 1H), 6.82 (d, J = 7.5 Hz, 1H), 6.02 (d, J = 8.0 Hz, 1H), 3.57 (d, J = 16.0 Hz, 1H), 3.46 (d, J= 16.0 Hz, 1H). |
| 79 | SS | 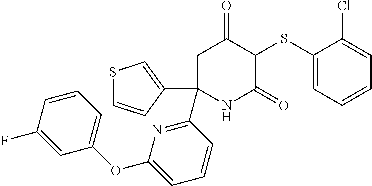 [SMILES: O=C1CC(c2ccsc2)(c2cccc(Oc3cccc(F)c3)n2)NC(=O)C1Sc1ccccc1Cl] | 3-(2-chlorophenyl)sulfanyl- 6-[6-(3-fluorophenoxy)- 2-pyridyl]-6-(3-thienyl) piperidine-2,4-dione was prepared in 7.9% yield according to the Example 3, Step A substituting 2-Chloro-4-fluoro-phenol for 3-fluorophenol | ¹H NMR (400 MHz, CD₃OD) δ 7.91 (dd, J = 8.0, 8.0 Hz, 1H), 7.45-7.39 (m, 1H), 7.26-7.20 (m, 1H), 7.01-6.94 (m, 6H), 6.87 (dd, J = 7.6, 7.6 Hz, 1H), 6.73 (dd, J = 7.6, 7.6 Hz, 1H), 6.01 (d, J = 8.0 Hz, 1H), 3.16 (d, J = 16.0 Hz, 1H), 3.07 (d, J = 16.0 Hz, 1H). |
| 80 | SS | 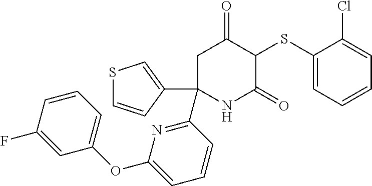 [SMILES: O=C1CC(c2ccsc2)(c2cccc(Oc3cccc(F)c3)n2)NC(=O)C1Sc1ccccc1Cl] | 3-(2-chlorophenyl)sulfanyl- 6-[6-(3-fluorophenoxy)- 2-pyridyl]-6-(3-thienyl) piperidine-2,4-dione was prepared in 7.9% yield according to the Example 3, Step A substituting 2-Chloro-4-fluoro-phenol for 3-fluorophenol | ¹H NMR (400 MHz, CD₃OD) δ 7.85 (dd, J = 8.0, 8.0 Hz, 1H), 7.39-7.33 (m, 3H), 7.22-7.15 (m, 2H), 7.03 (dd, J = 4.8, 1.2 Hz, 1H), 6.96-6.76 (m, 5H), 6.73 (dd, J = 8.0, 8.0 Hz, 1H), 6.08 (dd, J = 8.0, 1.2 Hz, 1H), 3.61 (d, J = 16.0 Hz, 1H), 3.29 (d, J = 16.0 Hz, 1H). |
| 81 | SS | 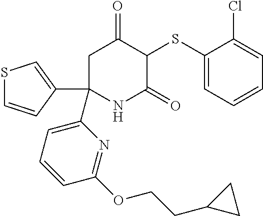 [SMILES: O=C1CC(c2ccsc2)(c2cccc(OCCC3CC3)n2)NC(=O)C1Sc1ccccc1Cl] | 3-((2-chlorophenyl)thio)- 6-(6-(2-cyclopropyl- ethoxy)pyridin-2-yl)-6- (thiophen-3-yl)piperidine- 2,4-dione was prepared in 34% yield according to the Example 2, Step A substituting propan-2-ol for 2-cyclopropylethanol. | ¹H NMR (400 MHz, (CD₃)₂SO) δ 11.55 (s, 1H), 8.45 (s, 1H), 7.73 (d, J = 8.4, 8.4 Hz, 1H), 7.48 (d, J = 8.4 Hz, 1H), 7.30-7.29 (m, 1H), 7.26 (d, J = 9.2 Hz, 1H), 7.18-7.16 (d, J = 7.2 Hz, 1H), 7.13-7.10 (m, 1H), 6.95-6.88 (m, 1H), 6.72 (d, J = 7.6 Hz, 2H), 5.83 (d, J = 8.0 Hz, 1H), 4.32-4.28 (m, 2H), 3.80 (d, J = 16.4 Hz, 1H), 3.30 (d, J = 16.4 Hz, 1H), 1.53-1.51 (m, 2H), 0.82-0.70 (m, 1H), 0.35-0.32 (m, 2H), 0.02-0.00 (m, 2H). |
| 82 | MD | 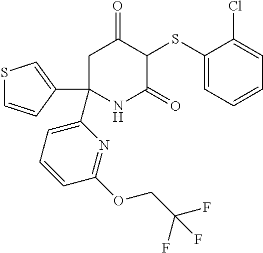 [SMILES: O=C1CC(c2ccsc2)(c2cccc(OCC(F)(F)F)n2)NC(=O)C1Sc1ccccc1Cl] | 3-(2-chlorophenyl)sulfanyl- 6-(3-thienyl)-6-[6-(2,2,2- trifluoroethoxy)-2- pyridyl]piperidine-2,4- dione was prepared in 20% yield according to Example 2, Step a substituting propan-2-ol for 2,2,2-trifluoroethanol. | ¹H NMR (400 MHz, (CD₃)₂SO) δ 11.66 (s, 1H), 8.63 (s, 1H), 7.93 (dd, J = 8.0 Hz, 1H), 7.53 (m, 1H), 7.38 (m,2H), 7.31 (d, J = 8.0 Hz, 1H), 7.19 (d, J = 5.2 Hz, 1H), 6.99 (m, 2H), 6.75 (dd, J = 8.4, 1.2 Hz, 1H), 5.80 (d, J = 8.4 Hz, 1H), 5.12 (m, 2H), 3.96 (d, J = 16.8 Hz, 1H), 3.40 (d, J = 16.8 Hz, 1H). |
| 83 | MD | 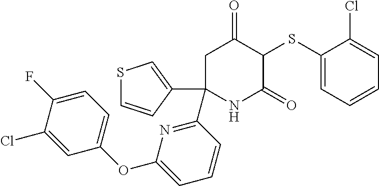 [SMILES: O=C1CC(c2ccsc2)(c2cccc(Oc3ccc(F)c(Cl)c3)n2)NC(=O)C1Sc1ccccc1Cl] | 6-(6-(3-chloro-4-fluoro- phenoxy)pyridin-2-yl)-3- ((2-chlorophenyl)thio)- 6-(thiophen-3-yl) piperidine-2,4-dione was prepared in 3% yield according to the Example 3, Step A substituting 2- Chloro-4-fluoro-phenol for 3-chloro-4-fluorophenol | ¹H NMR (400 MHz, CD₃OD) δ 7.92 (d, J = 8.0, 8.0 Hz, 1H), 7.44- 7.38 (m, 2H), 7.29-7.22 (m, 4H), 7.06-6.96 (m, 4H), 6.82 (dd, J = 7.2, 7.2 Hz, 1H), 6.02 (d, J = 8.0 Hz, 1H), 3.63 (d, J = 16.4 Hz, 1H), 3.34 (d, J = 16.4 Hz, 1H). |
| 84 | MD | 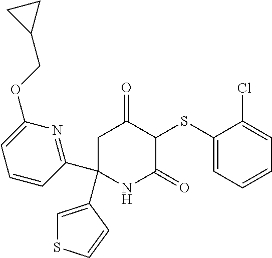 [SMILES: O=C1CC(c2ccsc2)(c2cccc(OCC3CC3)n2)NC(=O)C1Sc1ccccc1Cl] | 3-(2-chlorophenyl)sulfanyl- 6-[6-(cyclopropyl- methoxy)-2-pyridyl]-6- (3-thienyl)piperidine-2,4- dione was prepared in 6.5% yield according to the Example 2, Step A substituting propan-2-ol for cyclopropylmethanol | ¹H NMR (400 MHz, CD₃OD) δ 7.75 (dd, J = 8.0, 8.0 Hz, 1H), 7.45 (dd, J = 5.2, 2.8 Hz, 1H), 7.29-7.24 (m, 1H), 7.23 (d, J = 1.2 Hz, 1H), 7.17-7.15 (m, 1H), 6.98-6.94 (m, 1H), 6.77 (m, 1H), 5.99 (dd, J = 8.0, 1.2 Hz, 1H), 4.19 (m, 2H), 3.90 (d, J = 16.4 Hz, 1H), 3.47 (d, J = 16.4 Hz, 1H), 1.31-1.21 (m, 1H), 0.56- 0.52 (m, 2H), 0.32 (t, J = 2.0 Hz, 2H). |
| 85 | MD | 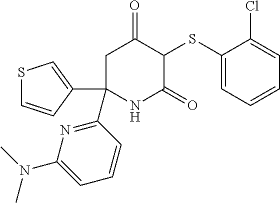 [SMILES: CN(C)c1cccc(C2(c3ccsc3)CC(=O)C(Sc3ccccc3Cl)C(=O)N2)n1] | 3-((2-chlorophenyl)thio)- 6-(6-(dimethylamino) pyridin-2-yl)-6-(thiophen- 3-yl)piperidine-2,4-dione was prepared in 8% yield according to the Example 4, Step A substituting cyclohexanamine for cyclopropanol. | ¹H NMR (400 MHz, (CD₃)₂SO) δ 8.39 (s, 1H), 7.58-7.54 (m, 1H), 7.50-7.48 (m, 1H), 7.37-7.36 (m, 1H), 7.31-7.28 (m, 1H), 7.18- 7.16 (m, 1H), 6.99-6.95 (m, 1H), 6.82 (d, J = 7.2 Hz, 1H), 6.76-6.72 (m, 1H), 6.57 (d, J = 8.4 Hz, 1H), 5.96 (dd, J = 8.0, 1.6 Hz, 1H), 3.97 (d, J = 16.4 Hz, 1H), 3.29 (d, J = 16.4 Hz, 1H), 3.04 (s, 6H). |
| 86 | SS | 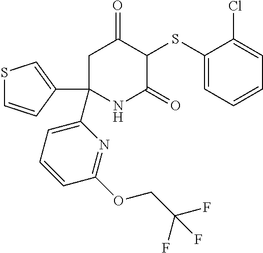 [SMILES: O=C1CC(c2ccsc2)(c2cccc(OCC(F)(F)F)n2)NC(=O)C1Sc1ccccc1Cl] | 3-(2-chlorophenyl)sulfanyl- 6-(3-thienyl)-6-[6-(2,2,2- trifluoroethoxy)-2- pyridyl]piperidine-2,4- dione was prepared in 6.6% yield according to the Example 2, Step A substituting propan-2-ol for 2,2,2-trifluoroethanol. | ¹H NMR (400 MHz, CD₃OD) δ 7.80 (dd, J = 7.6, 7.6 Hz, 1H), 7.39 (dd, J = 4.8, 2.8 Hz, 1H), 7.32-7.29 (m, 2H), 7.18 (dd, J = 4.8, 1.6 Hz, 2H), 6.91 (dd, J = 8.4, 8.4 Hz, 2H), 6.76 (dd, J = 7.2, 7.2 Hz, ,1H), 6.07 (d, J = 1.2 Hz, 1H), 4.99-4.89 (m, 2H), 3.65 (d, J = 16.0 Hz, 1H), 3.41 (d, J = 16.0 Hz, 1H). |
| 87 | SS | 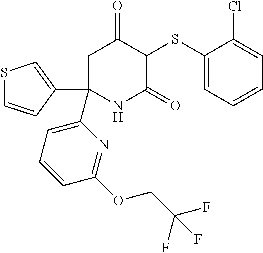 [SMILES: O=C1CC(c2ccsc2)(c2cccc(OCC(F)(F)F)n2)NC(=O)C1Sc1ccccc1Cl] | 3-(2-chlorophenyl)sulfanyl- 6-(3-thienyl)-6-[6- (2,2,2-trifluoroethoxy)-2- pyridyl]piperidine-2,4- dione was prepared in 6.6% yield according to the Example 2, Step A substituting propan-2-ol for 2,2,2-trifluoroethanol. | ¹H NMR (400 MHz, CD₃OD) δ 7.83 (dd, J = 7.6, 7.6 Hz, 1H), 7.43 (dd, J = 4.8, 2.8 Hz, 1H), 7.29-7.27 (m, 2H), 7.15 (dd, J = 5.2, 5.2 Hz, 2H), 6.87-6.82 (m, 2H), 6.72 (dd, J = 7.2, 7.2 Hz, 1H), 6.05 (d, J = 7.2 Hz, 1H), 4.95-4.88 (m, 2H), 3.58 (d, J = 16.0 Hz, 1H), 3.36 (d, J = 16.0 Hz, 1H). |
| 88 | MD | 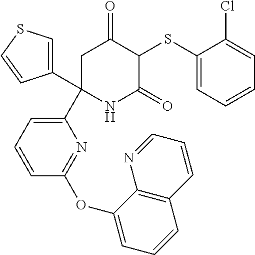 [SMILES: O=C1CC(c2ccsc2)(c2cccc(Oc3cccc4cccnc34)n2)NC(=O)C1Sc1ccccc1Cl] | 33-(2-chlorophenyl) sulfanyl-6-[6-(8- quinolyloxy)-2-pyridyl]- 6-(3-thienyl)piperidine- 2,4-dione was prepared in 11.6% yield according to the Example 3, Step A substituting chloro-4-fluoro-phenol for quinolin-8-ol. | ¹H NMR (400 MHz, (CD₃)₂SO) δ 8.65 (dd, J = 4.4, 1.6 Hz, 1H), 8.43 (dd, J = 8.4, 1.6 Hz, 1H), 7.89 (d, J = 7.2 Hz, 1H), 7.80 (d, J = 8.0 Hz, 1H), 7.64 (dd, J = 8.0, 8.0 Hz, 1H), 7.54- 7.52 (m, 2H), 7.26 (d, J = 7.2 Hz, 1H), 7.19-7.12 (m, 4H), 6.93 (d, J = 8.0 Hz, 1H), 6.87- 6.85 (m, 2H), 6.73 (dd, J = 8.0, 8.0 Hz 1H), 6.51 (d, J = 4.8 Hz, 1H), 6.00 (d, J = 8.0 Hz, 1H), 2.98 (d, J = 16.0 Hz, 1H), 2.83 (d, J = 16.0 Hz, 1H). |
| 89 | MD | 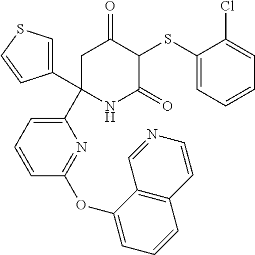 [SMILES: O=C1CC(c2ccsc2)(c2cccc(Oc3cccc4ccncc34)n2)NC(=O)C1Sc1ccccc1Cl] | 3-(2-chlorophenyl)sulfanyl- 6-[6-(8-iso- quinolyloxy)-2-pyridyl]-6- (3-thienyl)piperidine-2,4- dione was prepared in 6.9% yield according to the Example 3, Step A substituting 2- Chloro-4-fluoro-phenol for isoquinolin-8-ol | ¹H NMR (400 MHz, CD₃OD) δ 9.74 (s, 1H), 8.58-8.52 (m, 2H), 8.19-8.06 (m, 4H), 7.59 (d, J = 7.6 Hz, 1H), 7.51 (d, J = 7.6, 1H), 7.39-7.35 (m, 2H), 7.23 (d, J = 8.0 Hz, 1H), 7.11 (s, 1H), 6.97-6.90 (m, 1H), 6.84 (dd, J = 7.2, 7.2 Hz, 1H), 6.01 (d, J = 7.6 Hz, 1H), 3.42 (d, J = 16.8 Hz, 1H), 3.25 (d, J = 16.8 Hz, 1H). |
| 90 | MD | 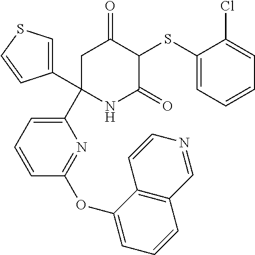 [SMILES: O=C1CC(c2ccsc2)(c2cccc(Oc3cccc4cnccc34)n2)NC(=O)C1Sc1ccccc1Cl] | 3-((2-chlorophenyl)thio)- 6-(6-(isoquinolin-5-yloxy) pyridin-2-yl)-6-(thiophen- 3-yl)piperidine-2,4-dione was prepared in 6% yield according to Example 3, Step A substituting 2- Chloro-4-fluoro-phenol for isoquinolin-5-ol | ¹H NMR (400 MHz, CD₃OD) δ 9.30 (s, 1H), 8.25 (d, J = 6.0 Hz, 1H), 8.02-7.94 (m, 2H), 7.73- 7.69 (m, 2H), 7.49 (dd, J = 7.6, 0.8 Hz, 1H), 7.38 (d, J = 6.0 Hz, 1H), 7.26-7.20 (m, 3H), 7.01-6.93 (m, 2H), 6.96 (dd, J = 8.0, 1.6 Hz, 1H), 3.32 (d, J = 16.8 Hz, 1H), 3.10 (d, J = 16.8 Hz, 1H). |
| 91 | MD | 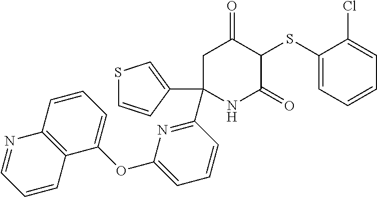 [SMILES: O=C1CC(c2ccsc2)(c2cccc(Oc3cccc4ncccc34)n2)NC(=O)C1Sc1ccccc1Cl] | 5-((2-chlorophenyl)thio)- 4-hydroxy-6′-(quinolin- 5-yloxy)-2-(thiophen-3- yl)-2,3-dihydro-[2,2′- bipyridin]-6(1H)-one was prepared in 13% yield according to the Example 3, Step A substituting 2-Chloro-4-fluoro- phenol for 4- fluorophenol. | ¹H NMR (400 MHz, (CD₃)₂SO) δ 8.95-8.94 (m, 1H), 8.25 (d, J = 8.4 Hz, 1H), 8.00-7.99 (m, 1H), 7.93 (d, J = 8.4 Hz, 1H), 7.70 (d, J = 7.6 , 7.6 Hz, 1H), 7.38- 7.35 (m, 1H), 7.30 (d, J = 7.6 Hz, 2H), 7.13 (d, J = 8.0 Hz, 2H), 7.01-6.90 (m, 1H), 6.84-6.78 (m, 2H), 5.95 (d, J = 8.0 Hz, 1H), 3.30 (d, J = 16.0 Hz, 1H), 3.2 (d, J = 16.0 Hz, 1H). |
| 92 | SS | 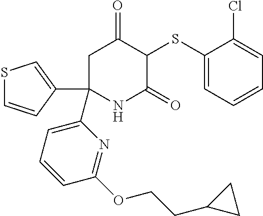 [SMILES: O=C1CC(c2ccsc2)(c2cccc(OCCC3CC3)n2)NC(=O)C1Sc1ccccc1Cl] | (6S)-3-((2-chlorophenyl) thio)-6-(6-(2-cyclopropyl- ethoxy)pyridin-2-yl)-6- (thiophen-3-yl)piperidine- 2,4-dione was prepared according to the Example 2, Step A substituting propan-2-ol for 2- cyclopropylethanol. | ¹H NMR (400 MHz, CD₃OD) δ 7.40 (d, J = 8.0 Hz, 1H), 7.42 (d, J = 8.0 Hz, 1H), 7.28-7.27 (m, 1H), 7.25-7.21 (m, 2H), 7.14 (d, J = 6.4 Hz, 1H), 7.12 (d, J = 8.0 Hz, 1H), 6.95-6.90 (m, 1H), 6.73 (d, J = 8.4 Hz, 1H), 6.02 (d, J = 8.0 Hz, 1H), 4.48- 4.41 (m, 2H), 3.70 (d, J= 16.4 Hz, 1H), 3.44 (d, J = 16.4 Hz, 1H), 1.61 (d, J = 6.8 Hz, 2H), 0.85- 0.75 (m, 1H), 0.42-0.38 (m, 2H), 0.07-0.02 (m, 2H). |
| 93 | SS | 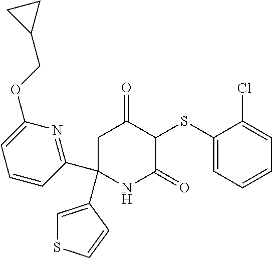 [SMILES: O=C1CC(c2ccsc2)(c2cccc(OCC3CC3)n2)NC(=O)C1Sc1ccccc1Cl] | 3-(2-chlorophenyl)sulfanyl- 6-[6-(cyclopropyl- methoxy)-2-pyridyl]-6- (3-thienyl)piperidine- 2,4-dione was prepared in 6.5% yield according to the Example 2, Step A substituting propan-2-ol for cyclopropylmethanol | ¹H NMR (400 MHz, CD₃OD) δ 7.36 (dd, J = 8.0, 8.0 Hz, 1H), 7.08 (dd, J = 5.2, 2.8 Hz, 1H), 6.99 (dd, J = 1.2, 1.2 Hz, 1H), 6.85-6.80 (m, 3H), 6.56-6.52 (m, 1H), 6.43-6.39 (m, 1H), 5.81 (d, J = 8.0 Hz, 1H), 3.87 (dd, J = 7.2, 2.0 Hz, 2H), 3.24 (d, J = 16.4 Hz, 1H), 3.05 (d, J = 16.4 Hz, 1H), 0.98-0.89 (m, 1H), 0.25-0.22 (m, 4H). |
| 94 | SS | 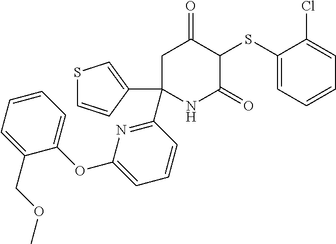 [SMILES: COCc1ccccc1Oc1cccc(C2(c3ccsc3)CC(=O)C(Sc3ccccc3Cl)C(=O)N2)n1] | 3-(2-chlorophenyl)sulfanyl- 6-[6-[4-fluoro-3-(trifluoro- methyl)phenoxy]-2- pyridyl]-6-(3-thienyl) piperidine-2,4-dione was prepared in 10% yield according to the Example 3, Step A substituting 2-chloro-4-fluoro-phenol for 2-(methoxymethyl) phenol | ¹H NMR (400 MHz, CD₃OD) δ 7.86 (dd, J = 8.0, 8.0 Hz, 1H), 7.48 (d, J = 8.4 Hz, 1H), 7.37- 7.32 (m, 5H), 7.13 (d, J = 7.6 Hz, 1H), 7.02-6.93 (m, 4H), 6.79 (dd, J = 8.0, 8.0 Hz, 1H), 5.97 (d, J = 8.0 Hz, 1H), 4.30 (s, 2H), 3.55 (d, J = 16.0 Hz, 1H), 3.26 (d, J = 16.4 Hz, 1H), 3.16 (s, 3H). |
| 95 | SS | 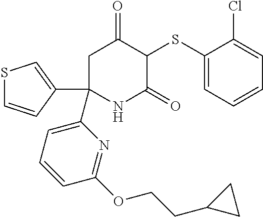 [SMILES: O=C1CC(c2ccsc2)(c2cccc(OCCC3CC3)n2)NC(=O)C1Sc1ccccc1Cl] | (6R)-3-((2-chlorophenyl) thio)-6-(6-(2-cyclo- propylethoxy)pyridin-2- yl)-6-(thiophen-3-yl) piperidine-2,4-dione was prepared according to the Example 2, Step A substituting propan-2-ol for 2-cyclopropylethanol. | ¹H NMR (400 MHz, CD₃OD) δ 7.70 (d, J = 8.0, 8.0 Hz, 1H), 7.43 (d, J = 8.0 Hz, 1H), 7.27-7.25 (m, 1H), 7.20 (d, J = 8.0 Hz, 1H), 7.15-7.12 (m, 2H), 6.98-6.90 (m, 1H), 6.75-6.73 (m, 2H), 5.98 (d, J = 8.0 Hz, 1H), 4.42- 4.41 (m, 2H), 3.89 (d, J = 16.4 Hz, 1H), 3.45 (d, J = 16.4 Hz, 1H), 1.61 (d, J = 6.8 Hz, 2H), 0.85-0.72 (m, 1H), 0.42-0.38 (m, 2H), 0.06-0.05 (m, 2H). |
| 96 | MD | 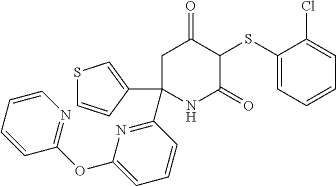 [SMILES: O=C1CC(c2ccsc2)(c2cccc(Oc3ccccn3)n2)NC(=O)C1Sc1ccccc1Cl] | 3-(2-chlorophenyl)sulfanyl- 6-[6-(2-pyridyloxy)-2- pyridyl]-6-(3-thienyl) piperidine-2,4-dione was prepared in 4% yield according to Example 3, Step A substituting 2- Chloro-4-fluoro-phenol for pyridin-2-ol. | ¹H NMR (400 MHz, (CD₃)₂SO) δ 11.53 (s, 1H), 8.51 (s, 1H), 8.23 (s, 1H), 7.99 (dd, J = 8.4, 8.4 Hz, 1H), 7.91 (m, 1H), 7.52 (m, 2H), 7.32 (m, 2H), 7.25 (m, 1H), 7.11 (m, 3H), 6.98 (dd, J = 6.8 Hz, 1H), 6.86 (dd, J = 7.2 Hz, 1H), 5.98 (d, J = 6.4 Hz, 1H), 3.69 (d, J = 16.8 Hz, 1H), 3.35 (d, J = 16.8 Hz, 1H). |
| 97 | SS | 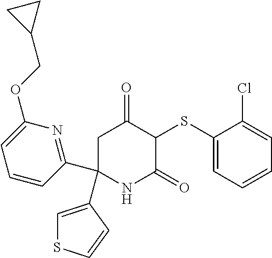 [SMILES: O=C1CC(c2ccsc2)(c2cccc(OCC3CC3)n2)NC(=O)C1Sc1ccccc1Cl] | 3-(2-chlorophenyl)sulfanyl- 6-[6-(cyclopropyl- methoxy)-2-pyridyl]-6- (3-thienyl)piperidine-2,4- dione was prepared in 6.5% yield according to the Example 2, Step A substituting propan-2-ol for cyclopropylmethanol | ¹H NMR (400 MHz, CD₃OD) δ 7.36 (dd, J = 8.0, 8.0 Hz, 1H), 7.08 (dd, J = 5.2, 2.8 Hz, 1H), 6.99 (s, 1H), 6.88-6.80 (m, 3H), 6.54 (dd, J = 8.0, 8.0 Hz, 1H), 6.43-6.39 (m, 2H), 5.81 (d, J = 8.0 Hz, 1H), 3.88 (dd, J = 7.2, 2.0 Hz, 2H), 3.25 (d, J = 16.4 Hz, 1H), 3.06 (d, J = 16.4 Hz, 1H), 0.98-0.91 (m, 1H), 0.23 (dd, J = 6.8, 4.8 Hz, 2H), 0.01 (t, J = 2.0 Hz, 2H). |
| 98 | MD | 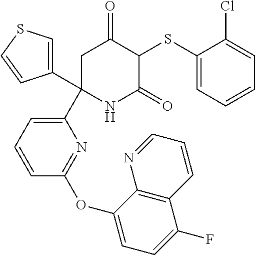 [SMILES: O=C1CC(c2ccsc2)(c2cccc(Oc3ccc(F)c4cccnc34)n2)NC(=O)C1Sc1ccccc1Cl] | 3-(2-chlorophenyl)sulfanyl- 6-[6-[(5-fluoro-8- quinolyl)oxy]-2-pyridyl]- 6-(3-thienyl)piperidine- 2,4-dione was prepared in 8.7% yield according to the Example 3, Step A substituting chloro-4- fluoro-phenol for 4- fluorophenol. | ¹H NMR (400 MHz, (CD₃)₂SO) δ 8.71 (d, J = 4.0 Hz, 1H), 8.54 (d, J = 8.0 Hz, 1H), 7.87 (dd, J = 8.0, 8.0 Hz, 1H) 7.63 (d, J = 2.8 Hz, 1H), 7.54-7.50 (m, 2H), 7.31-7.26 (m, 3H), 7.22 (d, J = 2.8 Hz, 1H), 7.05-7.03 (m, 2H), 6.93 (dd, J = 8.0, 8.0 Hz, 1H), 6.55 (d, J = 4.0 Hz, 1H), 5.95 (d, J = 8.0 Hz, 1H), 3.10 (d, J = 16.0 Hz, 1H), 2.91 (d, J = 16.0 Hz, 1H). |
| 99 | SS | 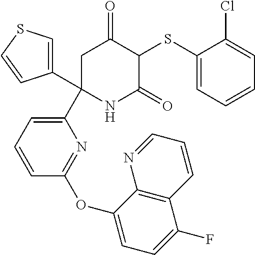 [SMILES: O=C1CC(c2ccsc2)(c2cccc(Oc3ccc(F)c4cccnc34)n2)NC(=O)C1Sc1ccccc1Cl] | 3-((2-chlorophenyl)thio)- 6-(6-(quinolin-8-yloxy) pyridin-2-yl)-6-(thiophen- 3-yl)piperidine-2,4-dione was prepared according to methods described therein | ¹H NMR (400 MHz, (CD₃)₂SO) δ 8.67 (d, J = 2.4 Hz, 1H), 8.46 (d, J = 7.2Hz, 1H), 7.92-7.83 (m, 2H), 7.66 (dd, J = 4.0, 4.0 Hz, 1H), 7.55-7.54 (m, 2H), 7.31- 7.29 (m, 3H), 7.28 (d, J = 7.6 Hz, 2H), 7.01-6.95 (m, 3H), 6.82 (dd, J = 8.0, 8.0 Hz, 1H), 6.60 (d, J = 4.8 Hz, 1H), 5.98 (d, J = 7.6 Hz, 1H), 3.20 (d, J = 16.0 Hz, 1H), 2.97 (d, J = 16.0 Hz, 1H). |
| 100 | SS | 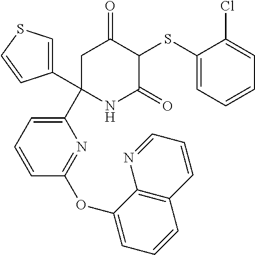 [SMILES: O=C1CC(c2ccsc2)(c2cccc(Oc3cccc4cccnc34)n2)NC(=O)C1Sc1ccccc1Cl] | 3-((2-chlorophenyl)thio)- 6-(6-(quinolin-8-yloxy) pyridin-2-yl)-6-(thiophen- 3-yl)piperidine-2,4-dione was prepared according to methods described therein | ¹H NMR (400 MHz, (CD₃)₂SO) δ 8.67 (dd, J = 4.0, 1.6 Hz, 1H), 8.45 (d, J = 6.8 Hz, 1H), 7.92-7.86 (m, 2H), 7.65 (dd, J = 8.0, 8.0 Hz, 1H), 7.56-7.53 (m, 2H), 7.29-7.28 (m, 2H), 7.27 (dd, J = 1.2, 1.2 Hz, 1H), 6.97 (d, J = 8.0 Hz, 1H), 6.92- 6.91 (m, 2H), 6.78 (dd, J = 8.0, 8.0 Hz, 1H), 6.57 (d, J = 4.4 Hz, 1H), 6.00 (d. J = 7.6 Hz, 1H), 3.10 (d, J = 16.0 Hz, 1H), 2.90 (d, J = 16.0 Hz, 1H). |
| 101 | MD | 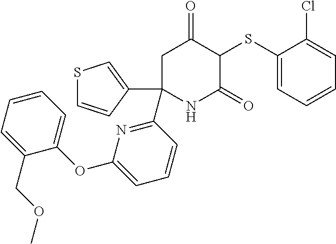 [SMILES: COCc1ccccc1Oc1cccc(C2(c3ccsc3)CC(=O)C(Sc3ccccc3Cl)C(=O)N2)n1] | 3-((2-chlorophenyl)thio)- 6-(6-(2-(methoxymethyl) phenoxy)pyridin-2-yl)- 6-(thiophen-3- yl)piperidine-2,4-dione was prepared in 19% yield according to the Example 3, Step A substituting propan-2-ol for 2- (methoxymethyl)phenol | ¹H NMR (400 MHz, CD₃OD) δ 7.90 (dd, J = 8.0, 8.0 Hz, 1H), 7.51 (d, J = 8.4 Hz, 1H), 7.38-7.30 (m, 5H), 7.27 (d, J = 7.6 Hz, 1H), 7.06- 7.00 (m, 4H), 6.82 (dd, J = 8.0, 8.0 Hz, 1H), 6.03 (d, J = 8.0 Hz, 1H), 4.33 (s, 2H), 3.52 (d, J = 16.0 Hz, 1H), 3.34 (d, J = 16.4 Hz, 1H), 3.20 (s, 3H). |
| 102 | MD | 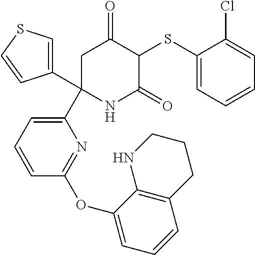 [SMILES: O=C1CC(c2ccsc2)(c2cccc(Oc3cccc4c3NCCC4)n2)NC(=O)C1Sc1ccccc1Cl] | 3-(2-chlorophenyl)sulfanyl- 6-[6-(1,2,3,4-tetrahydro- quinolin-8-yloxy)-2- pyridyl]-6-(3-thienyl) piperidine-2,4-dione was prepared in 14.2% yield according to the Example 3, Step A substituting 2-chloro-4-fluoro-phenol for 1,2,3,4-tetrahydro- quinolin-8-ol. | ¹H NMR (400 MHz, (CD₃)₂SO) δ 9.49 (s, 1H ), 8.38 (s, 1H ), 7.56- 7.44 (m, 3H), 7.32-7.25 (m, 2H), 7.01-6.94 (m, 3H), 6.80 (d, J = 8.0 Hz 1H), 7.71-6.69 (m, 2H), 6.30 (d, J = 8.0 Hz 1H), 5.89 (d, J = 8.0 Hz, 1H), 4.21-3.79 (m, 2H), 3.38 (d, J = 5.2 Hz, 2H), 2.70-2.63 (m, 2H), 1.78-1.75 (m, 2H). |
| 103 | MD | 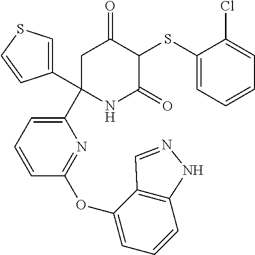 [SMILES: O=C1CC(c2ccsc2)(c2cccc(Oc3cccc4[nH]ncc34)n2)NC(=O)C1Sc1ccccc1Cl] | 3-(2-chlorophenyl)sulfanyl- 6-[6-(1H-indazol-4-yloxy)- 2-pyridyl]-6-(3-thienyl) piperidine-2,4-dione was prepared in 11% yield according to Example 3, Step A substituting 2- Chloro-4-fluoro-phenol for 1H-indazol-4-ol. | ¹H NMR (400 MHz, (CD₃)₂SO) δ 8.35 (s, 1H), 7.99-7.97 (m, 2H), 7.95 (d, J = 4.4 Hz, 1H), 7.61 (d, J = 7.6 Hz, 1H), 7.44-7.42 (m, 2H), 7.26 (d, J = 8.4 Hz, 1H), 7.24 (m, 1H), 7.20 (d, J = 5.2 Hz, 1H), 6.76 (m, 1H), 6.57 (m, 2H), 6.06 (d, J = 7.2 Hz, 1H), 3.49 (d, J = 15.2 Hz, 1H), 3.10 (d, J = 15.2 Hz, 1H). |
| 104 | MD | 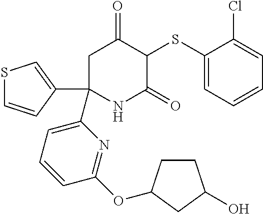 [SMILES: O=C1CC(c2ccsc2)(c2cccc(OC3CCC(O)C3)n2)NC(=O)C1Sc1ccccc1Cl] | 3-((2-chlorophenyl)thio)- 6-(6-((3-hydroxycyclo- pentyl)oxy)pyridin-2-yl)- 6-(thiophen-3-yl) piperidine-2,4-dione was prepared in 35% yield according to the Example 2, Step A substituting propan-2-ol for cyclo- pentane-1,3-diol | ¹H NMR (400 MHz, CD₃OD) δ 8.46 (d, J = 11.2 Hz, 1H), 7.73 (dd, J = 8.4, 4.0 Hz, 1H), 7.52 (dd, J = 2.4, 2.4 Hz, 1H), 7.35- 7.17 (m, 4H), 6.96 (dd, J = 4.0, 4.0 Hz, 1H), 6.71-6.69 (m, 2H), 5.86 (dd, J = 9.2, 1.6 Hz, 1H), 5.46-5.45 (m, 1H), 4.62 (s, 1H), 4.25 (s, 1H), 3.90 (s, 1H), 2.19- 1.83 (m, 4H), 1.62-1.53 (m, 2H). |
| 105 | MD | 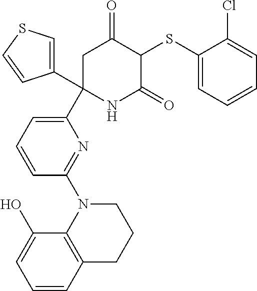 [SMILES: O=C1CC(c2ccsc2)(c2cccc(N3CCCc4cccc(O)c43)n2)NC(=O)C1Sc1ccccc1Cl] | 3-(2-chlorophenyl)sulfanyl- 6-[6-(8-hydroxy-3,4- dihydro-2H-quinolin-1- yl)-2-pyridyl]-9-(3- thienyl)piperidine-2,4- dione was prepared in 3.3% yield according to the Example 3, Step A substituting chloro-4- fluoro-phenol for 1,2,3,4-tetrahydro- quinolin-8-ol. | ¹H NMR (400 MHz, (CD₃)₂SO) δ 11.61 (s, 1H), 9.45 (s, 1H), 8.34 (dd, J = 8.0, 8.0 Hz, 1H), 7.52- 7.39 (m, 3H), 7.26-7.21 (m, 2H), 6.96-6.90 (m 3H), 6.66-6.46 (m, 3H), 6.25 (d, J = 8.0 Hz, 1H), 5.84 (d, J = 8.0 Hz, 1H), 4.16- 3.75 (m, 3H), 2.66-2.59 (m, 1H), 3.17 (d, J = 5.2 Hz, 2H), 1.72- 1.70 (m, 2H). |
| 106 | SS | 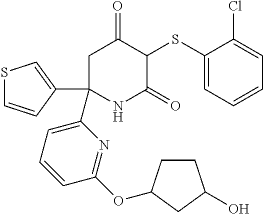 [SMILES: O=C1CC(c2ccsc2)(c2cccc(OC3CCC(O)C3)n2)NC(=O)C1Sc1ccccc1Cl] | 3-((2-chlorophenyl)thio)- 6-(6-((3-hydroxy- cyclopentyl)oxy)pyridin- 2-yl)-6-(thiophen-3- yl)piperidin-2,4-dione was separated from example 97. | ¹H NMR (400 MHz,CD₃OD) δ 8.46 (d, J = 11.2 Hz, 1H), 7.73 (dd, J = 8.4, 4.0 Hz, 1H), 7.52 (dd, J = 2.4, 2.4 Hz, 1H), 7.35- 7.16 (m, 4H), 6.96 (d, J = 4.0 Hz, 1H), 6.71-6.69 (m, 2H), 5.88- 5.83 (m, 1H), 5.30-5.25 (m, 1H), 4.62 (s, 1H), 4.10 (s, 1H), 3.93- 3.87 (m, 1H), 2.40-2.37 (m, 1H), 1.99-1.48 (m, 5H). |
| 107 | MD | 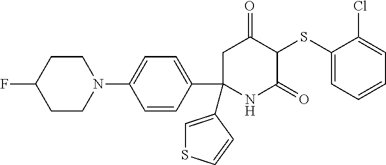 [SMILES: O=C1CC(c2ccc(N3CCC(F)CC3)cc2)(c2ccsc2)NC(=O)C1Sc1ccccc1Cl] | 3-(2-chlorophenyl)sulfanyl-6- [4-(4-fluoro-1- piperidyl)phenyl]-6-(3- thienyl)piperidine-2,4-dione was prepared in 9% yield according to Example 7, Step H substituting 2-methylmorpholine for 4-fluoropiperidine. | ¹H NMR (400 MHz, (CD₃)₂SO) δ 7.47-7.46 (m, 2H), 7.27 (s, 1H), 7.26-7.21 (m, 3H), 7.09 (d, J = 4.0 Hz, 1H), 6.89-6.87 (m, 3H), 6.70 (d, J = 8.4 Hz, 1H),5.99 (d, J = 7.2 Hz, 1H), 4.88-4.74 (m, 1H), 3.35-3.30 (m, 2H), 3.13- 3.05 (m, 4H), 1.96-1.90 (m, 2H), 1.77-1.73 (m, 2H). |
| 108 | SS | 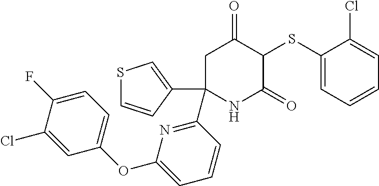 [SMILES: O=C1CC(c2ccsc2)(c2cccc(Oc3ccc(F)c(Cl)c3)n2)NC(=O)C1Sc1ccccc1Cl] | 6-(6-(3-chloro-4-fluoro- phenoxy)pyridin-2-yl)- 3-((2-chlorophenyl)thio)- 6-(thiophen-3-yl) piperidine-2,4-dione was separated from example 76. | ¹H NMR (400 MHz, CD₃OD) δ 7.89 (d, J = 8.0, 8.0 Hz, 1H), 7.40-7.35 (m, 2H), 7.24-7.19 (m, 4H), 7.03-7.01 (m, 4H), 6.78 (dd, J = 7.2, 7.2 Hz, 1H), 6.02 (d, J = 8.0 Hz, 1H), 3.55 (d, J = 16.4 Hz, 1H), 3.31 (d, J = 16.4 Hz, 1H). |
| 109 | SS | 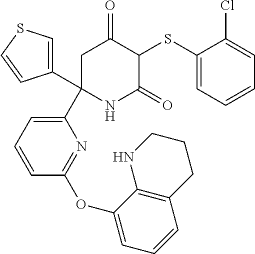 [SMILES: O=C1CC(c2ccsc2)(c2cccc(Oc3cccc4c3NCCC4)n2)NC(=O)C1Sc1ccccc1Cl] | 3-((2-chlorophenyl)thio)- 6-(6-((1,2,3,4-tetrahydro- quinolin-8-yl)oxy)pyridin- 2-yl)-6-(thiophen-3- yl)piperidine-2,4-dione was prepared according to methods described therein | ¹H NMR (400 MHz, (CD₃)₂SO) δ 7.84 (dd, J = 8.0, 8.0 Hz, 1H), 7.45 (dd, J = 4.8, 2.0 Hz, 1H), 7.33-7.26 (m, 3H), 7.07 (d, J = 4.4 Hz, 1H), 6.93 (dd, J = 2.0, 2.0 Hz, 1H), 6.79-6.69 (m, 4H), 6.45 (dd, J = 8, 8 Hz, 1H), 6.00 (d, J = 7.6 Hz, 1H), 5.12 (s, 1H), 3.12 (d, J = 5.2 Hz, 2H), 2.73-2.72 (m, 2H), 1.80-1.78 (m, 2H) 1.20-1.23 (m, 2H). |
| 110 | MD | 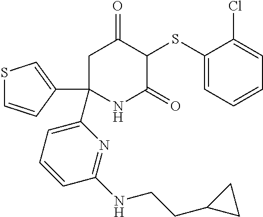 [SMILES: O=C1CC(c2ccsc2)(c2cccc(NCCC3CC3)n2)NC(=O)C1Sc1ccccc1Cl] | 3-(2-chlorophenyl)sulfanyl- 6-[6-(2-cyclopropylethyl- amino)-2-pyridyl]-6-(3- thienyl)piperidine-2,4-dione was prepared in 21% yield according to the Example 4, Step A substituting cyclo- hexanamine for 2- cyclopropylethanamine | ¹H NMR (400 MHz, CD₃OD) δ 7.74 (dd, J = 7.6, 7.6 Hz, 1H), 7.56 (d, J = 2.0 Hz, 1H), 7.55 (d, J = 2.8 Hz, 1H), 7.16 (d, J = 5.6 Hz, 1H), 6.93 (dd, J = 8.0, 8.0 Hz, 2H), 6.76 (d, J = 7.4 Hz, 1H), 6.43 (d, J = 7.6 Hz, 1H), 5.97 (d, J = 8.0 Hz, 1H), 3.47 (d, J = 16.0 Hz, 1H), 3.41 (d, J = 16.0 Hz, 1H), 3.38 (t, J = 7.4 Hz, 2H), 1.43-1.38 (m, 2H), 0.66-0.62 (m, 1H), 0.40-0.35 (m, 2H), 0.02-0.00 (m, 2H). |
| 111 | SS | 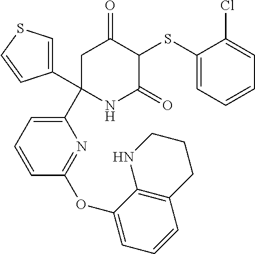 [SMILES: O=C1CC(c2ccsc2)(c2cccc(Oc3cccc4c3NCCC4)n2)NC(=O)C1Sc1ccccc1Cl] | 3-((2-chlorophenyl)thio)- 6-(6-((1,2,3,4-tetrahydro- quinolin-8-yl)oxy)pyridin- 2-yl)-6-(thiophen-3- yl)piperidine-2,4-dione was prepared according to methods described therein | ¹H NMR (400 MHz, (CD₃)₂SO) δ 7.84 (dd, J = 8.0, 8.0 Hz, 1H), 7.45 (dd, J = 4.8, 2.0 Hz, 1H), 7.33-7.26 (m, 3H), 7.07 (d, J = 4.4 Hz, 1H), 6.93 (dd, J = 2.0, 2.0 Hz, 1H), 6.79-6.69 (m, 4H), 6.45 (dd, J = 8.0, 8.0 Hz, 1H), 6.00 (d, J = 7.6 Hz, 1H), 5.12 (s, 1H), 3.12 (d, J = 5.2 Hz, 2H), 2.70-2.71 (m, 2H), 1.79- 1.76 (m, 2H), 1.20-1.23 (m, 2H). |
| 112 | SS | 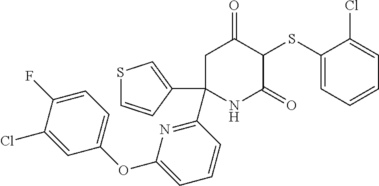 [SMILES: O=C1CC(c2ccsc2)(c2cccc(Oc3ccc(F)c(Cl)c3)n2)NC(=O)C1Sc1ccccc1Cl] | 6-(6-(3-chloro-4-fluoro- phenoxy)pyridin-2-yl)- 3-((2-chlorophenyl)thio)- 6-(thiophen-3-yl) piperidine-2,4-dione was separated from example 76. | ¹H NMR (400 MHz, CD₃OD) δ 7.89 (d, J = 8.0, 8.0 Hz, 1H), 7.40-7.24 (m, 2H), 7.22-7.20 (m, 4H), 7.03-7.01 (1H, 4H), 6.78 (dd, J = 7.2, 7.2 Hz, 1H), 6.01 (d, J = 8.0 Hz, 1H), 3.56 (d, J = 16.4 Hz, 1H), 3.31 (d, J = 16.4 Hz, 1H). |
| 113 | MD | 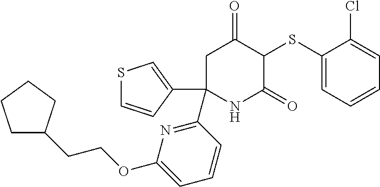 [SMILES: O=C1CC(c2ccsc2)(c2cccc(OCCC3CCCC3)n2)NC(=O)C1Sc1ccccc1Cl] | 3-((2-chlorophenyl)thio)- 6-(6-(2-cyclopentyl- ethoxy)pyridin-2-yl)-6- (thiophen-3-yl)piperidine- 2,4-dione was prepared in 31% yield according to the Example 2, Step A substituting propan-2-ol for 2-cyclopentylethanol | ¹H NMR (400 MHz, CD₃OD) δ 7.68 (dd, J = 8.4, 4.0 Hz, 1H), 7.46 (dd, J = 8.0, 1.2 Hz, 1H), 7.27-7.12 (m, 4H), 6.93 (dd, J = 4.0, 4.0 Hz, 1H), 6.75-6.73 (m, 2H), 5.95 (d, J = 6.8 Hz, 1H), 4.39-4.36 (m, 2H), 3.90 (d, J = 16.4 Hz, 1H), 3.44 (d, J = 16.0 Hz, 1H), 1.93-1.48 (m, 9 H), 1.13-1.11 (m, 2H). |
| 114 | MD | 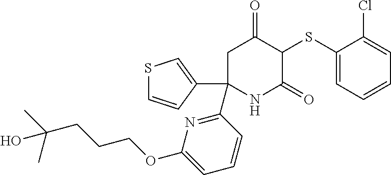 [SMILES: CC(C)(O)CCCOc1cccc(C2(c3ccsc3)CC(=O)C(Sc3ccccc3Cl)C(=O)N2)n1] | 3-((2-chlorophenyl)thio)- 6-(6-((4-hydroxy-4-methyl- pentyl)oxy)pyridin-2-yl)- 6-(thiophen-3-yl) piperidine-2,4-dione was prepared in 28% yield according to the Example 2, Step A substituting propan-2-ol for 4-methyl- pentane-1,4-diol | ¹H NMR (400 MHz, CD₃OD) δ 11.56 (s, 1H), 8.47 (s, 1H), 7.74 (dd, J = 8.0, 4.0 Hz, 1H), 7.48 (dd, J = 5.2, 3.2 Hz, 1H), 7.33- 7.14 (m, 4H), 6.87 (dd, J = 4.0, 4.0 Hz, 1H), 6.74-6.71 (m, 2H), 4.30-4.21 (m, 2H), 3.91 (d, J = 16.0 Hz, 1H), 3.34 (d, J = 16.0 Hz, 1H), 1.72-1.68 (m, 2H), 1.37- 1.39 (m, 2H), 1.03 (d, J = 2.8 Hz, 6H). |
| 115 | MD | 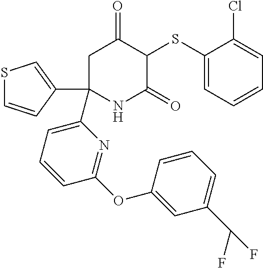 [SMILES: O=C1CC(c2ccsc2)(c2cccc(Oc3cccc(C(F)F)c3)n2)NC(=O)C1Sc1ccccc1Cl] | 3-(2-chlorophenyl)sulfanyl- 6-[6-[3-(difluoromethyl) phenoxy]-2-pyridyl]-6-(3- thienyl)piperidine-2,4-dione was prepared in 33% yield according to the Example 3, Step A substituting 2- Chloro-4-fluoro-phenol for 3-(difluoromethyl)phenol | ¹H NMR (400 MHz, (CD₃)₂SO) δ 8.47 (s, 1H), 7.97 (dd, J = 7.6, 7.6 Hz, 1H), 7.53 (dd, J = 7.8, 7.8 Hz, 1H), 7.47 (dd, J = 5.2, 3.2 Hz, 1H), 7.44 (d, J = 7.6 Hz, 1H), 7.40 (d, J = 7.2 Hz, 1H), 7.30 (dd, J = 8.0, 1.2 Hz, 1H), 7.27-7.22 (m, 3H), 7.12-6.83 (m, 4H), 6.81-6.75 (m, 1H), 5.91 (dd, J = 8.4, 1.2 Hz, 1H), 3.56 (d, J = 16.4 Hz, 1H), 3.25 (d, J = 16.4 Hz, 1H). |
| 116 | MD | 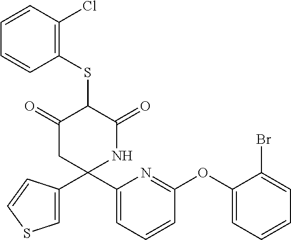 [SMILES: O=C1CC(c2ccsc2)(c2cccc(Oc3ccccc3Br)n2)NC(=O)C1Sc1ccccc1Cl] | 6-[6-(2-bromophenoxy)-2- pyridyl]-3-(2-chlorophenyl) sulfanyl-6-(3-thienyl) piperidine-2,4-dione was prepared in 6% yield according to the Example 3, Step A substituting 2- chloro-4-fluoro-phenol for 2-bromophenol. | ¹H NMR (400 MHz, CD₃OD) δ 7.88 (dd, J = 7.6, 7.6 Hz, 1H), 7.67 (d, J = 6.8 Hz, 1H), 7.39- 7.30 (m, 3H), 7.15-7.13 (m, 4H), 7.03 (d, J = 2.8 Hz, 1H), 6.96 (d, J = 2.8 Hz, 1H), 6.79 (d, J = 7.6 Hz, 1H), 5.99 (d, J = 8.0 Hz, 1H), 3.51 (d, J = 16.0 Hz, 1H), 3.21 (d, J = 16.0 Hz, 1H). |
| 117 | SS | 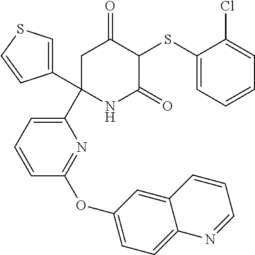 [SMILES: O=C1CC(c2ccsc2)(c2cccc(Oc3ccc4ncccc4c3)n2)NC(=O)C1Sc1ccccc1Cl] | 3-(2-chlorophenyl)sulfanyl- 6-[6-(6-quinolyloxy)-2- pyridyl]-6-(3-thienyl) piperidine-2,4-dione was prepared in 10% yield according to the Example 3, Step A substituting 2- chloro-4-fluoro-phenol for quinolin-6-ol. | ¹H NMR (400 MHz, CD₃OD) δ 9.30 (s, 1H), 8.25 (dd, J = 7.6, 7.6 Hz, 1H), 8.02-7.96 (m, 2H), 7.69 (d, J = 7.6 Hz, 1H), 7.50 (d, J = 7.2 Hz, 1H), 7.37 (d, J = 7.6 Hz, 1H), 7.24-7.22 (m, 3H), 7.01-7.00 (m, 2H), 6.77 (d, J = 5.2 Hz, 1H), 5.96 (d, J = 8.0 Hz, 1H), 3.32 (d, J = 16.0 Hz, 1H), 3.10 (d, J = 16.0 Hz, 1H). |
| 118 | MD | 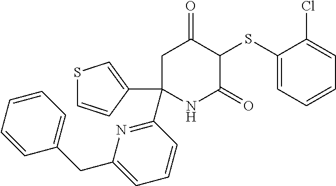 [SMILES: O=C1CC(c2ccsc2)(c2cccc(Cc3ccccc3)n2)NC(=O)C1Sc1ccccc1Cl] | 6-(6-benzylpyridin-2-yl)- 3-((2-chloropnenyl)thio)- 6-(thiophen-3-yl) piperidine-2,4-dione was prepared in 8% yield according to the Example 5, Step B substituting- Bromomethyl-3-fluoro- benzene for (bromomethyl)benzene | ¹H NMR (400 MHz, CD₃OD) δ 7.76 (dd, J = 8.0, 8.0 Hz, 1H), 7.27-7.22 (m, 6H), 7.20-7.09 (m, 2H), 6.86 (dd, J = 8.0, 8.0 Hz, 1H), 6.56 (dd, J = 8.0, 8.0 Hz, 1H), 5.86 (dd, J = 8.0, 1.6 Hz, 1H), 4.18 (s, 2H), 3.98 (d, J = 16.4 Hz, 1H), 3.49 (d, J = 16.0 Hz, 1H). |
| 119 | MD | 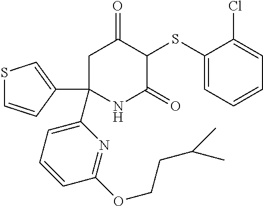 [SMILES: CC(C)CCOc1cccc(C2(c3ccsc3)CC(=O)C(Sc3ccccc3Cl)C(=O)N2)n1] | 3-(2-chlorophenyl)sulfanyl- 6-(6-isopentyloxy-2- pyridyl)-6-(3-thienyl) piperidine-2,4-dione was prepared in 7.8% yield according to the Example 2, Step A substituting propan- 2-ol for 3- methylbutan-1-ol. | ¹H NMR (400 MHz, (CD₃)₂SO) δ 11.61 (s, 1H), 8.53 (s, 1H), 7.77 (dd, J = 8.0, 8.0 Hz, 1H), 7.52 (dd, J = 8.0, 4.0 Hz 1H), 7.35 (dd, J = 1.2, 1.2 Hz, 1H), 7.30 (dd, J = 5.2, 1.2 Hz, 1H), 7.22 (d, J = 8.0 Hz, 1H), 7.16 (d, J = 4.0 Hz, 1H), 6.97 (dd, J = 8.0, 8.0 Hz, 1H), 6.78-6.71 (m, 2H), 5.84 (d, J = 8.0 Hz, 1H),4.35-4.29 (m, 2H), 3.93 (d, J = 16.0 Hz, 1H), 3.37 (d, J = 16.0 Hz, 1H), 1.75-1.68 (m, 1H), 1.58-1.53 (m, 2H), 0.92- 0.83 (m, 6H). |
| 120 | SS | 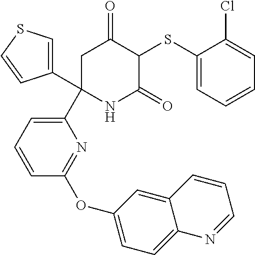 [SMILES: O=C1CC(c2ccsc2)(c2cccc(Oc3ccc4ncccc4c3)n2)NC(=O)C1Sc1ccccc1Cl] | 3-(2-chlorophenyl)sulfanyl- 6-[6-(6-quinolyloxy)- 2-pyridyl]-6-(3-thienyl) piperidine-2,4-dione was prepared in 10% yield according to example 3, Step A substituting 2- chloro-4-fluoro-phenol for quinolin-6-ol. | ¹H NMR (400 MHz, CD₃OD) δ 9.30 (s, 1H), 8.25 (dd, J = 7.6, 7.6 Hz, 1H), 8.02-7.96 (m, 2H), 7.70 (d, J = 7.6 Hz, 1H), 7.54 (d, J = 7.2 Hz, 1H), 7.37 (d, J = 7.6 Hz, 1H), 7.26-7.20 (m, 3H), 7.01-7.00 (m, 2H), 6.77 (d, J = 5.2 Hz, 1H), 5.96 (d, J = 8.0 Hz, 1H), 3.32 (d, J = 16.0 Hz, 1H), 3.10 (d, J = 16.0 Hz, 1H). |
| 121 | MD | 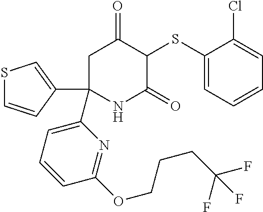 [SMILES: O=C1CC(c2ccsc2)(c2cccc(OCCCC(F)(F)F)n2)NC(=O)C1Sc1ccccc1Cl] | 3-(2-chlorophenyl)sulfanyl- 6-(3-thienyl)-6-[6- (4,4,4-trifluorobutoxy)-2- pyridyl]piperidine-2,4- dione was prepared in 11.7% yield according to the Example 2, Step A substituting propan-2-ol for 3,3,3-trifluoropropan- 1-ol. | ¹H NMR (400 MHz, (CD₃)₂SO) δ 8.47 (s, 1H) 7.80 (dd, J = 8.0, 8.0 Hz, 1H), 7.51 (d, J = 3.6 Hz, 1H), 7.36 (dd, J = 1.2, 1.2 Hz, 1H), 7.29-7.24 (m, 2H), 7.17 (d, J = 5.2 Hz, 1H), 6.96 (dd, J = 8.0, 8.0 Hz, 1H), 6.80 (d, J = 8.0 Hz, 1H), 6.73 (dd, J = 8.0, 8.0 Hz, 1H), 5.84 (d, J = 4.0 Hz, 1H), 4.37 (t, J = 8.0 Hz, 1H), 3.89 (d, J = 16.0 Hz, 1H), 3.36 (t, J = 8.0 Hz, 1H), 2.39-2.32 (m, 2H), 1.94-1.88 (m, 2H). |
| 122 | MD | 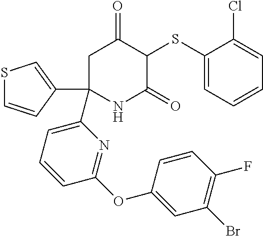 [SMILES: O=C1CC(c2ccsc2)(c2cccc(Oc3ccc(F)c(Br)c3)n2)NC(=O)C1Sc1ccccc1Cl] | 6-[6-(3-bromo-4-fluoro- phenoxy)-2-pyridyl]-3- (2-chlorophenyl)sulfanyl- 6-(3-thienyl)piperidine- 2,4-dione was prepared in 6.8% yield according to the Example 3, Step A substituting 2-Chloro-4- fluoro-phenol for 3- bromo-4-fluorophenol | ¹H NMR (400 MHz, CD₃OD) δ 7.99 (dd, J = 8.0, 8.0 Hz, 1H), 7.42-7.33 (m, 3H), 7.24-7.18 (m, 3H), 7.08-7.02 (m, 3H), 6.95 (dd, J = 8.0, 8.0 Hz, 1H), 6.89 (dd, J = 8.0, 4.0 Hz, 1H), 5.99 (d, J = 8.0 Hz, 1H), 3.60 (d, J = 16.0 Hz, 1H), 3.31 (d, J = 16.0 Hz, 1H). |
| 123 | MD | 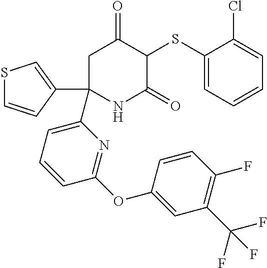 [SMILES: O=C1CC(c2ccsc2)(c2cccc(Oc3ccc(F)c(C(F)(F)F)c3)n2)NC(=O)C1Sc1ccccc1Cl] | 3-(2-chlorophenyl)sulfanyl- 6-[6-[4-fluoro-3- (trifluoromethyl)phenoxy]- 2-pyridyl]-6-(3-dione was prepared in 15% yield according to the Example 3, Step A substituting 2- chloro-4-fluoro-phenol for 4-fluoro-3-(trifluoro- methyl)phenol. | ¹H NMR (400 MHz, CD₃OD) δ 7.93 (dd, J = 7.6, 7.6 Hz, 1H), 7.40-7.37 (m, 5H), 7.34 (d, J = 2.8 Hz, 1H), 7.32 (d, J = 2.8 Hz, 1H), 7.17 (d, J = 7.6 Hz, 1H), 7.16-7.07 (m, 2H), 6.80 (d, J = 7.6 Hz, 1H), 5.99 (d, J = 8.0 Hz, 1H), 3.56 (d, J = 16.0 Hz, 1H), 3.29 (d, J = 16.0 Hz, 1H). |
| 124 | SS | 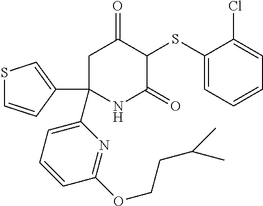 [SMILES: CC(C)CCOc1cccc(C2(c3ccsc3)CC(=O)C(Sc3ccccc3Cl)C(=O)N2)n1] | 3-((2-chlorophenyl)thio)- 6-(6-(isopentyloxy) pyridin-2-yl)-6-(thiophen- 3-yl)piperidine-2,4-dione was prepared according to methods described therein | ¹H NMR (400 MHz, (CD₃)₂SO) δ 11.61 (s, 1H), 8.53 (s, 1H), 7.77 (dd, J = 8.0, 8.0 Hz, 1H), 7.52 (dd, J = 8.0, 4.0 Hz 1H), 7.35 (dd, J = 1.2, 1.2 Hz, 1H), 7.30 (dd, J = 5.2, 1.2 Hz, 1H), 7.22 (d, J = 8.0 Hz, 1H), 7.16 (d, J = 4.0 Hz, 1H), 6.97 (dd, J = 8.0, 8.0 Hz, 1H), 6.78- 6.71 (m, 2H), 5.84 (d, J = 8.0 Hz, 1H), 4.35-4.29 (m, 2H), 3.93 (d, J = 16.0 Hz, 1H), 3.27 (d, J = 16.0 Hz, 1H), 1,73-1.69 (m, 1H), 1.58- 1.53 (m, 2H), 0.92-0.83 (m, 6H). |
| 125 | SS | 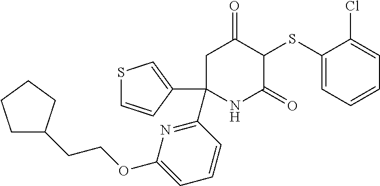 [SMILES: O=C1CC(c2ccsc2)(c2cccc(OCCC3CCCC3)n2)NC(=O)C1Sc1ccccc1Cl] | 3-((2-chlorophenyl)thio)- 6-(6-(2-cyclopentyl- ethoxy)pyridin-2-yl)- 6-(thiophen-3-yl) piperidine-2,4-dione was separated from example 113. | ¹H NMR (400 MHz, CD₃OD) δ 7.71 (dd, J = 8.4, 4.0 Hz, 1H), 7.43 (dd, J = 2.8, 2.8 Hz, 1H), 7.26 (d, J = 2.0 Hz, 1H), 7.26 (d, J = 2.0 Hz, 1H), 7.20 (d, J = 2.8 Hz, 1H), 7.14-7.12 (m, 2H), 6.92 (dd, J = 2.0, 2.0 Hz, 1H), 6.75- 6.73 (m, 2H), 5.95 (dd, J = 8.4, 1.6 Hz, 1H), 4.40-4.33 (m, 2H), 3.89 (d, J = 16.4 Hz, 1H), 3.44 (d, J = 16.4 Hz, 1H), 1.91-1.48 (m, 9H), 1.13-1.10 (m, 2H). |
| 126 | SS | 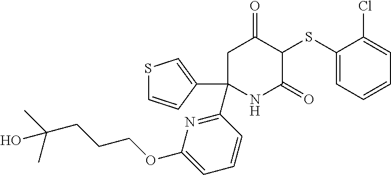 [SMILES: CC(C)(O)CCCOc1cccc(C2(c3ccsc3)CC(=O)C(Sc3ccccc3Cl)C(=O)N2)n1] | 3-(2-chlorophenyl)sulfanyl- 6-[6-(4-hydroxy-4- methyl-pentoxy)-2- pyridyl]-6-(3-thienyl) piperidine-2,4-dione was prepared according to methods described therein | ¹H NMR (400 MHz, CD₃OD) δ 7.69 (dd, J = 8.0, 8.0 Hz, 1H), 7.41 (dd, J = 5.2, 3.2 Hz, 1H), 7.27 (d, J = 1.2 Hz, 1H), 7.16- 7.12 (m, 2H), 6.88 (dd, J = 4.0, 4.0 Hz, 1H), 6.74-6.72 (m, 2H), 4.38-4.33 (m, 2H), 3.79 (d, J = 16.0 Hz, 1H), 3.42 (d, J = 16.4 Hz, 1H), 1.83-1.79 (m, 2H), 1.58-1.54 (m, 2H), 1.15 (d, J = 2.4 Hz, 6H). |
| 127 | SS | 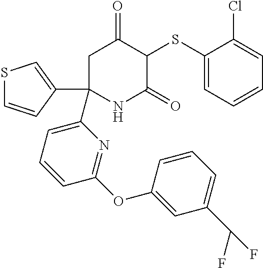 [SMILES: O=C1CC(c2ccsc2)(c2cccc(Oc3cccc(C(F)F)c3)n2)NC(=O)C1Sc1ccccc1Cl] | 3-(2-chlorophenyl)sulfanyl- 6-[6-[3-(difluoromethyl) phenoxy]-2-pyridyl]-6- (3-thienyl)piperidine- 2,4-dione was separated from example 115. | ¹H NMR (400 MHz, CD₃OD) δ 7.90 (dd, J = 7.6, 7.6 Hz, 1H), 7.50 (dd, J = 8.0, 8.0 Hz, 1H), 7.41-7.33 (m, 3H), 7.24-7.20 (m, 3H), 7.18 (dd, J = 1.6, 1.6 Hz, 1H), 7.04 (d, J = 8.4 Hz, 1H), 7.01 (dd, J = 4.2, 1.2 Hz, 1H), 6.98-6.93 (m, 1H), 6.82-6.78 (m, 1H), 6.74 (t, J = 56.0 Hz, 1H), 6.01 (dd, J = 7.6, 1.2 Hz, 1H), 3.61 (d, J = 16.8 Hz, 1H), 3.30 (d, J = 16.8 Hz, 1H). |
| 128 | SS | 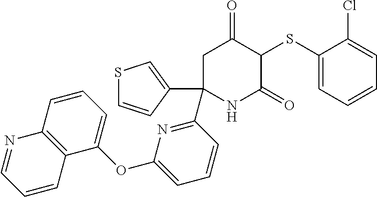 [SMILES: O=C1CC(c2ccsc2)(c2cccc(Oc3cccc4ncccc34)n2)NC(=O)C1Sc1ccccc1Cl] | 3-(2-chlorophenyl)sulfanyl- 6-[6-(5-quinolyloxy)-2- pyridyl]-6-(3-thienyl) piperidine-2,4-dione was prepared in 6.5% yield according to the Example 3, Step A substituting 2-chloro-4-fluoro- phenol for quinolin-5-ol. | ¹H NMR (400 MHz, (CD₃)₂SO) δ 8.94 (dd, J = 2.8, 1.2 Hz, 1H), 8.25 (d, J = 8.4 Hz, 1H), 7.99- 7.92 (m, 2H), 7.77 (dd, J = 8.0, 8.0 Hz, 1H), 7.46-7.29 (m, 5H), 7.14-7.12 (m, 2H), 6.95 (dd, J = 8.0, 8.0 Hz, 1H), 6.83-6.77 (m, 2H), 5.93 (d, J = 8.0 Hz, 1H), 3.40 (d, J = 16.0 Hz, 1H), 3.11 (d, J= 16.0 Hz, 1H). |
| 129 | MD | 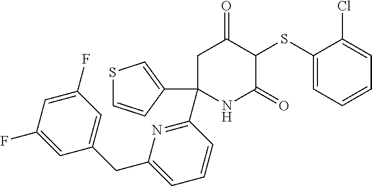 [SMILES: O=C1CC(c2ccsc2)(c2cccc(Cc3cc(F)cc(F)c3)n2)NC(=O)C1Sc1ccccc1Cl] | 3-((2-chlorophenyl)thio)- 6-(6-(3,5-difluorobenzyl) pyridin-2-yl)-6-(thiophen- 3-yl)piperidine-2,4-dione was prepared in 26 yield according to the Example 5, Step B substituting 1-Bromomethyl-3-fluoro- benzene for 1-(bromo- methyl)-3,5-difluoro- benzene | ¹H NMR (400 MHz, CD₃OD) δ 7.77 (dd, J = 8.0. 8.0 Hz, 1H), 7.46 (d, J = 8.0 Hz, 1H), 7.41 (dd, J = 0.8, 0.4 Hz, 1H), 7.30 (d, J = 8.0 Hz, 1H), 7.22-7.17 (m, 2H), 7.08 (dd, J = 5.6, 0.8 Hz, 1H), 6.90-6.84 (m, 3H), 6.66 (dd, J = 8.0, 8.0 Hz, 1H), 6.54 (dd, J = 8.0, 8.0 Hz, 1H), 5.78 (dd, J = 8.0, 4.0 Hz, 1H), 4.16 (s, 2H), 3.97 (d, J = 16.8 Hz, 1H), 3.47 (d, J = 16.8 Hz, 1H). |
| 130 | MD | 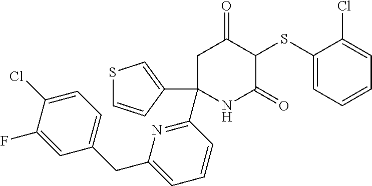 [SMILES: O=C1CC(c2ccsc2)(c2cccc(Cc3ccc(Cl)c(F)c3)n2)NC(=O)C1Sc1ccccc1Cl] | 6-(6-(4-chloro-3-fluoro- benzyl)pyridin-2-yl)-3- ((2-chlorophenyl)thio)-6- (thiophen-3-yl)piperidine- 2,4-dione was prepared in 4% yield according to the Example 5, Step B substituting 1-Bromo- methyl-3-fluoro-benzene for 4-(bromomethyl)-1- chloro-2-fluorobenzene | ¹H NMR (400 MHz, CD₃OD) δ 7.69 (dd, J = 8.0. 8.0 Hz, 1H), 7.41 (d, J = 8.0 Hz, 1H), 7.33 (dd, J = 8.0, 8.0 Hz, 1H), 7.29-7.27 (m, 2H), 7.19-7.17 (m, 2H), 7.08- 7.07 (m, 2H), 6.74 (dd, J = 8.0, 8.0 Hz, 1H), 6.53 (dd, J = 8.0, 8.0 Hz, 1H), 5.99 (d, J = 8.8 Hz, 1H), 4.14 (s, 2H), 3.51 (d, J = 16.0 Hz, 1H), 3.36 (d, J = 16.0 Hz, 1H). |
| 131 | SS | 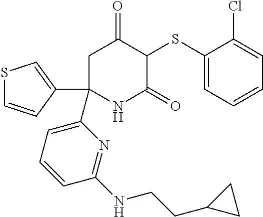 [SMILES: O=C1CC(c2ccsc2)(c2cccc(NCCC3CC3)n2)NC(=O)C1Sc1ccccc1Cl] | 3-(2-chlorophenyl)sulfanyl- 6-[6-(2-cyclopropyl- ethylamino)-2-pyridyl]-6- (3-thienyl)piperidine- 2,4-dione was prepared in 21% yield according to the Example 4, Step A substituting cyclo- hexanamine for 2- cyclopropylethanamine | ¹H NMR (400 MHz, CD₃OD) δ 7.73 (dd, J = 7.6, 7.6 Hz, 1H), 7.56 (d, J = 2.0 Hz, 1H), 7.55 (d, J = 2.8 Hz, 1H), 7.16 (d, J = 5.4 Hz, 2H), 6.94 (dd, J = 8.0, 8.0 Hz, 2H), 6.76 (d, J = 7.4 Hz, 1H), 6.43 (d, J = 7.5 Hz, 1H), 5.97 (d, J = 8.0 Hz, 1H), 3.48 (d, J = 16.0 Hz, 1H), 3.42 (d, J = 16.0 Hz, 1H), 3.37 (t, J = 7.4 Hz, 2H), 1.41-1.39 (m, 2H), 0.65-0.61 (m, 1H), 0.38-0.37 (m, 2H), 0.01-0.00 (m, 2H) |
| 132 | MD | 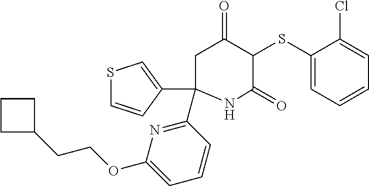 [SMILES: O=C1CC(c2ccsc2)(c2cccc(OCCC3CCC3)n2)NC(=O)C1Sc1ccccc1Cl] | 3-((2-chlorophenyl)thio)- 6-(6-(2-cyclobutyl- ethoxy)pyridin-2-yl)-6- (thiophen-3-yl)piperidine- 2,4-dione was prepared in 34% yield according to the Example 1, Step A substituting propan-2-ol for 2-cyclobutylethanol | ¹H NMR (400 MHz, CD₃OD) δ 7.69 (dd, J = 8.0, 4.0 Hz, 1H), 7.45 (dd, J = 5.2, 1.2 Hz, 1H), 7.30-7.20 (m, 2H), 7.15-7.12 (m, 2H), 6.93 (dd, J = 8.0, 4.0 Hz, 1H), 6.73-6.71 (m, 2H), 5.97 (dd, J = 7.6, 1.2 Hz, 1H), 4.36- 4.26 (m, 2H), 3.88 (d, J = 16.0 Hz, 1H), 3.44 (d, J = 16.0 Hz, 1H), 2.45-2.41 (m, 1H), 2.02- 2.00 (m, 2H), 1.83-1.64 (m, 6H). |
| 133 | SS | 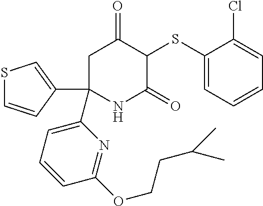 [SMILES: CC(C)CCOc1cccc(C2(c3ccsc3)CC(=O)C(Sc3ccccc3Cl)C(=O)N2)n1] | 3-(2-chlorophenyl)sulfanyl- 6-(6-isopentyloxy-2- pyridyl)-6-(3-thienyl) piperidine-2,4-dione was separated from example 112. | ¹H NMR (400 MHz, (CD₃)₂SO) δ 11.61 (s, 1H), 8.53 (s, 1H), 7.77 (dd, J = 8.0, 8.0 Hz, 1H), 7.52 (dd, J = 8.0, 4.0 Hz 1H), 7.35 (dd, J = 1.2, 1.2 Hz, 1H), 7.30 (dd, J = 5.2, 1.2 Hz, 1H), 7.22 (d, J = 8.0 Hz, 1H), 7.16 (d, J = 4.0 Hz, 1H), 6.97 (dd, J = 8.0, 8.0 Hz, 1H), 6.78-6.71 (m, 2H), 5.84 (d, J = 8.0 Hz, 1H), 4.35-4.29 (m, 2H), 3.83 (d, J = 16.0 Hz, 1H), 3.30 (d, J = 16.0 Hz, 1H), 1.75-1.68 (m, 1H), 1.58-1.53 (m, 2H), 0.90- 0.86 (m, 6H). |
| 134 | SS | 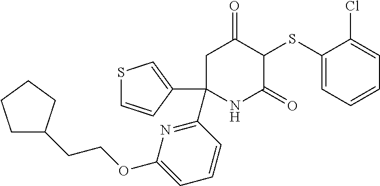 [SMILES: O=C1CC(c2ccsc2)(c2cccc(OCCC3CCCC3)n2)NC(=O)C1Sc1ccccc1Cl] | 3-(2-chlorophenyl)sulfanyl- 6-[6-(2-cyclopentyl- ethoxy)-2-pyridyl]-6-(3- thienyl)piperidine-2,4- dione was prepared according to methods described therein | ¹H NMR (400 MHz, CD₃OD) δ 7.71 (dd, J = 8.4, 4.0 Hz, 1H), 7.43 (dd, J = 2.8, 2.8 Hz, 1H), 7.26 (d, J = 2.0 Hz, 1H), 7.26 (d, J = 2.0 Hz, 1H), 7.20 (d, J = 2.8, 1H), 7.14-7.12 (m, 2H), 6.92 (dd, J = 2.0, 2.0 Hz, 1H), 6.75-6.73 (m, 2H), 5.95 (dd, J = 8.0, 1.6 Hz, 1H), 4.40-4.33 (m, 2H), 3.89 (d, J = 16.4 Hz, 1H), 3.44 (d, J = 16.4 Hz, 1H), 1.91-1.48 (m, 9H), 1.13-1.10 (m, 2H). |
| 135 | SS | 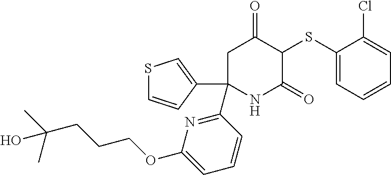 [SMILES: CC(C)(O)CCCOc1cccc(C2(c3ccsc3)CC(=O)C(Sc3ccccc3Cl)C(=O)N2)n1] | 3-(2-chlorophenyl)sulfanyl- 6-[6-(4-hydroxy-4- methyl-pentoxy)-2- pyridyl]-6-(3-thienyl) piperidine-2,4-dione was prepared according to methods described therein | ¹H NMR (400 MHz, CD₃OD) δ 7.68 (dd, J = 8.0, 8.0 Hz, 1H), 7.41 (dd, J = 5.2, 3.2 Hz, 1H), 7.27 (d, J = 1.2 Hz, 1H), 7.16- 7.12 (m, 2H), 6.88 (dd, J = 4.0, 4.0 Hz, 1H), 6.74-6.72 (m, 2H), 4.37-4.35 (m, 2H), 3.68 (d, J = 16.4 Hz, 1H), 3.39 (d, J = 16.0 Hz, 1H), 1.80-1.79 (m, 2H), 1.59-1.55 (m, 2H), 1.15 (d, J = 1.6 Hz, 6H). |
| 136 | SS | 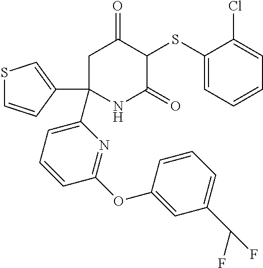 [SMILES: O=C1CC(c2ccsc2)(c2cccc(Oc3cccc(C(F)F)c3)n2)NC(=O)C1Sc1ccccc1Cl] | 3-(2-chlorophenyl)sulfanyl- 6-[6-[3-(difluoro- methyl)phenoxy]-2- pyridyl]-6-(3-thienyl) piperidine-2,4-dione was separated from example 115. | ¹H NMR (400 MHz, CD₃OD) δ 7.88 (dd, J = 7.6, 7.6 Hz, 1H), 7.50 (dd, J = 8.0, 8.0 Hz, 1H), 7.38-7.34 (m, 3H), 7.22-7.18 (m, 4H), 7.02-7.00 (m, 2H), 6.94- 6.90 (m, 1H), 6.80-6.76 (m, 1H), 6.74 (t, J = 56.0 Hz, 1H), 6.04 (dd, J = 7.6, 1.2 Hz, 1H), 3.56 (d, J = 16.8 Hz, 1H), 3.30 (d, J = 16.8 Hz, 1H). |
| 137 | SS | 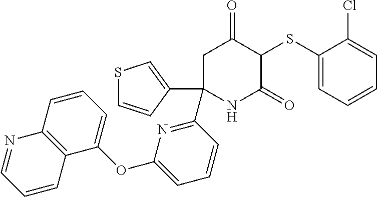 [SMILES: O=C1CC(c2ccsc2)(c2cccc(Oc3cccc4ncccc34)n2)NC(=O)C1Sc1ccccc1Cl] | 3-(2-chlorophenyl)sulfanyl- 6-[6-(5-quinolyloxy)- 2-pyridyl]-6-(3-thienyl) piperidine-2,4-dione was prepared in 4.2% yield according to the Example 3, Step A substituting chloro-4-fluoro-phenol for quinolin-5-ol. | ¹H NMR (400 MHz, (CD₃)₂SO) δ 8.94 (dd, J = 2.7, 1.2 Hz, 1H), 8.25 (d, J = 8.0 Hz, 1H), 7.98- 7.92 (m, 2H), 7.78 (dd, J = 8.0, 8.0 Hz, 1H), 7.45-7.43 (m, 2H), 7.34-7.30 (m, 3H), 7.12 (m, 2H), 6.94 (dd, J = 8.0, 8.0 Hz, 1H), 6.82-6.75 (m, 2H), 5.95 (d, J = 7.2 Hz, 1H), 3.08 (d, J = 16.0 Hz, 1H), 2.52 (d, J = 16.0 Hz, 1H). |
| 138 | SS | 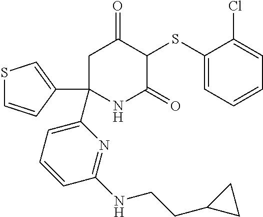 [SMILES: O=C1CC(c2ccsc2)(c2cccc(NCCC3CC3)n2)NC(=O)C1Sc1ccccc1Cl] | 3-(2-chlorophenyl)sulfanyl- 6-[6-(2-cyclopropyl- ethylamino)-2-pyridyl]- 6-(3-thienyl)piperidine- 2,4-dione was prepared in 21% yield according to the Example 4, Step A substituting cyclo- hexanamine for 2- cyclopropylethanamine | ¹H NMR (400 MHz, CD₃OD) δ 7.74 (dd, J = 7.6, 7.6 Hz, 1H), 7.56 (d, J = 2.0 Hz, 1H), 7.55 (d, J = 2.8 Hz, 1H), 7.16 (d, J = 5.4 Hz, 2H,), 6.94 (dd, J = 8.0, 8.0 Hz, 2H), 6.76 (d, J = 7.4 Hz, 1H), 6.43 (d, J = 7.5 Hz, 1H), 5.97 (d, J = 8.0 Hz, 1H), 3.47 (d, J = 16.0 Hz, 1H), 3.44 (d, J = 16.0 Hz, 1H), 3.38 (t, J = 7.4 Hz, 2H), 1.41-1.39 (m, 2H), 0.65-0.61 (m, 1H), 0.38-0.37 (m, 2H), 0.01-0.00 (m, 2H) |
| 139 | MD | 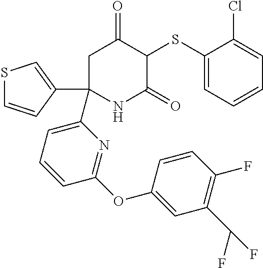 [SMILES: O=C1CC(c2ccsc2)(c2cccc(Oc3ccc(F)c(C(F)F)c3)n2)NC(=O)C1Sc1ccccc1Cl] | 3-(2-chlorophenyl)sulfanyl- 6-[6-[3-(difluoro- methyl)-4-fluoro-phenoxy]- 2-pyridyl]-6-(3-thienyl) piperidine-2,4-dione was prepared in 7.2% yield according to the Example 3, Step A substituting 2-Chloro-4-fluoro-phenol for 4-(difluoromethyl)- 3-fluorophenol | ¹H NMR (400 MHz, CD₃OD) δ 7.90 (dd, J = 8.0, 8.0 Hz, 1H), 7.40-7.35 (m, 2H), 7.30 (d, J = 4.0 Hz, 1H), 7.24-7.18 (m, 4H), 7.09-6.93 (m, 4H), 6.79 (dd, J = 8.0, 8.0 Hz, 1H), 6.00 (d, J = 8.0 Hz, 1H), 3.57 (d, J = 16.0 Hz, 1H), 3.27 (d, J = 16.0 Hz, 1H). |
| 140 | MD | 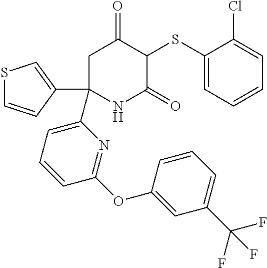 [SMILES: O=C1CC(c2ccsc2)(c2cccc(Oc3cccc(C(F)(F)F)c3)n2)NC(=O)C1Sc1ccccc1Cl] | 3-(2-chlorophenyl)sulfanyl- 6-(3-thienyl)-6-[6-[3- (trifluoromethyl)phenoxy]- 2-pyridyl]piperidine-2,4- dione was prepared in 11.2% yield according to the Example 3, Step A substituting 2-chloro-4- fluoro-phenol for 3- (trifluoromethyl)phenol. | ¹H NMR (400 MHz, (CD₃)₂SO) δ 8.52 (s, 1H), 8.02 (dd, J = 8.0, 8.0 Hz, 1H), 7.66-7.58 (m, 2H), 7.50- 7.47 (m, 3H), 7.40 (d, J = 8.0 Hz, 1H), 7.32 (dd, J = 8.0, 4.0 Hz, 1H), 7.26-7.27 (m, 1H), 7.09 (d, J = 8.0 Hz, 1H), 7.04-6.98 (m, 2H), 6.81 (dd, J = 8.0, 4.0 Hz, 1H), 5.95 (d, J = 8.0 Hz, 1H), 3.96 (d, J = 16.0 Hz, 1H), 3.28 (d, J = 16.0 Hz, 1H). |
| 141 | SS | 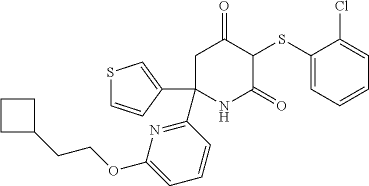 [SMILES: O=C1CC(c2ccsc2)(c2cccc(OCCC3CCC3)n2)NC(=O)C1Sc1ccccc1Cl] | 3-(2-chlorophenyl)sulfanyl- 6-[6-(2-cyclobutyl- ethoxy)-2-pyridyl]-6-(3- thienyl)piperidine-2,4- dione was prepared according to methods described therein | ¹H NMR (400 MHz, CD₃OD) δ 7.69 (dd, J = 8.0, 4.0 Hz, 1H), 7.45 (dd, J = 5.2, 1.2 Hz, 1H), 7.30-7.20 (m, 2H), 7.15-7.12 (m, 2H), 6.93 (dd, J = 8.0, 4.0 Hz, 1H), 6.73-6.71 (m, 2H), 5.97 (dd, J = 7.6, 1.2 Hz, 1H), 4.30-4.26 (m, 2H), 3.88 (d, J = 16.0 Hz, 1H), 3.44 (d, J = 16.0 Hz, 1H), 2.45-2.41 (m, 1H), 2.03-1.64 (m, 8H). |
| 142 | SS | 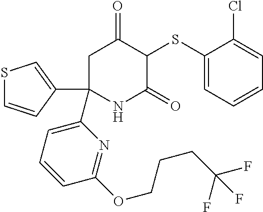 [SMILES: O=C1CC(c2ccsc2)(c2cccc(OCCCC(F)(F)F)n2)NC(=O)C1Sc1ccccc1Cl] | 3-(2-chlorophenyl)sulfanyl- 6-(3-thienyl)-6-[6-(4,4,4- trifluorobutoxy)-2- pyridyl]piperidine-2,4- dione was separated from example 121. | ¹H NMR (400 MHz, (CD₃)₂SO) δ 7.80 (dd, J = 8.0, 8.0 Hz, 1H), 7.50 (d, J = 4.8 Hz, 1H), 7.36 (dd, J = 1.2, 1.2 Hz, 1H), 7.29- 7.24 (m, 2H), 7.17 (d, J = 4.8 Hz, 1H), 6.93 (dd, J = 4.0, 4.0 Hz, 1H), 6.80-6.73 (m, 2H), 5.87 (d, J = 8.0 Hz, 1H), 4.37 (t, J = 5.6 Hz, 2H), 3.80 (d, J = 16.0 Hz, 1H), 3.34 (d, J = 16.0 Hz, 1H), 2.39-2.32 (m, 2H), 1.92-1.88 (m, 2H). |
| 143 | SS | 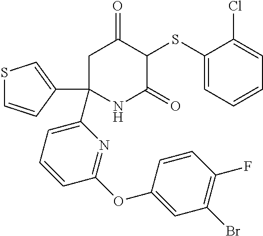 [SMILES: O=C1CC(c2ccsc2)(c2cccc(Oc3ccc(F)c(Br)c3)n2)NC(=O)C1Sc1ccccc1Cl] | 6-[6-(3-bromo-4-fluoro- phenoxy)-2-pyridyl]-3-(2- chlorophenyl)sulfanyl-6- (3-thienyl)piperidine-2,4- dione was prepared in 6.8% yield according to the Example 3, Step A substituting 2-Chloro-4- fluoro-phenol for 3- bromo-4-fluorophenol | ¹H NMR (400 MHz, CD₃OD) δ 7.86 (dd, J = 8.0, 8.0 Hz, 1H), 7.38-7.33 (m, 3H), 7.25-7.14 (m, 3H), 7.11-7.07 (m, 1H), 7.03 (d, J = 4.0 Hz, 1H), 6.96 (d, J = 8.0 Hz, 1H), 6.96 (dd, J = 8.0, 8.0 Hz, 1H), 6.74 (dd, J = 8.0, 8.0 Hz, 1H), 6.09 (d, J = 8.0 Hz, 1H), 3.42 (d, J = 16.0 Hz, 1H), 3.27 (d, J = 16.0 Hz, 1H). |
| 144 | SS | 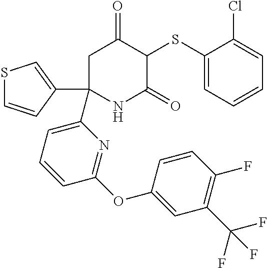 [SMILES: O=C1CC(c2ccsc2)(c2cccc(Oc3ccc(F)c(C(F)(F)F)c3)n2)NC(=O)C1Sc1ccccc1Cl] | 3-(2-chlorophenyl)sulfanyl- 6-[6-[4-fluoro-3- (trifluoromethyl)phenoxy]- 2-pyridyl]-6-(3-thienyl) piperidine-2,4-dione was prepared in 15% yield according to the Example 3, Step A substituting 2- chloro-4-fluoro-phenol for 4-fluoro-3-(trifluoro- methyl)phenol. | ¹H NMR (400 MHz, CD₃OD) δ 7.93 (dd, J = 7.6, 7.6 Hz, 1H), 7.40-7.37 (m, 5H), 7.34 (d, J = 2.8 Hz, 1H), 7.32 (d, J = 2.8 Hz, 1H), 7.17 (d, J = 7.6 Hz, 1H), 7.16-7.07 (m, 2H), 6.80 (d, J = 7.6 Hz, 1H), 5.99 (d, J = 8.0 Hz, 1H), 3.56 (d, J = 16.0 Hz, 1H), 3.28 (d, J = 16.0 Hz, 1H). |
| 145 | MD | 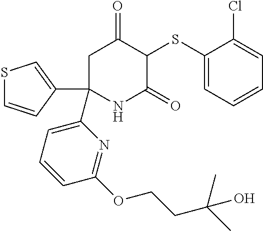 [SMILES: CC(C)(O)CCOc1cccc(C2(c3ccsc3)CC(=O)C(Sc3ccccc3Cl)C(=O)N2)n1] | 3-(2-chlorophenyl)sulfanyl- 6-[6-(3-hydroxy-3- methyl-butoxy)-2-pyridyl]- 6-(3-thienyl)piperidine- 2,4-dione was prepared in 13% yield according to the Example 2, Step A substituting propan-2-ol for 3-methylbutane- 1,3-diol. | ¹H NMR (400 MHz, CD₃OD) δ 7.74 (dd, J = 7.6, 7.6 Hz, 1H), 7.46 (dd, J = 5.0, 3.0 Hz, 1H), 7.29 (dd, J = 3.0, 1.5 Hz, 1H), 7.24 (dd, J = 8.0, 1.3 Hz, 1H), 7.20-7.16 (m, 2H), 6.98-6.94 (m, 1H), 6.78 (d, J = 8.0 Hz, 1H), 6.78-6.74 (m, 1H), 5.97 (dd, J = 8.0, 1.6 Hz, 1H), 4.59- 4.44 (m, 1H), 3.97 (d, J = 16.3 Hz, 1H), 3.48 (d, J = 16.6 Hz, 1H), 1.93 (t, J = 7.2 Hz, 2H), 1.25 (d, J = 5.3 Hz, 6H). |
| 146 | SS | 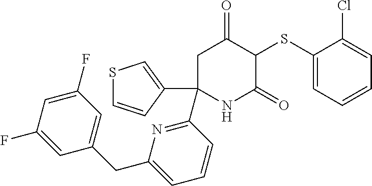 [SMILES: O=C1CC(c2ccsc2)(c2cccc(Cc3cc(F)cc(F)c3)n2)NC(=O)C1Sc1ccccc1Cl] | 3-(2-chlorophenyl)sulfanly- 6-[6-[(3,5-difluorophenyl) methyl]-2-pyridyl]-6-(3- thienyl)piperidine-2,4- dione was prepared according to methods described therein | ¹H NMR (400MHz, CD₃OD) δ 7.79 (dd, J = 8.0, 8.0 Hz, 1H), 7.48-7.46 (m, 2H), 7.30-7.17 (m, 4H), 6.90-6.88 (m, 3H), 6.68 (dd, J = 8.0, 8.0 Hz, 1H), 6.56 (dd, J = 8.0, 8.0 Hz, 1H), 5.83 (d, J = 8.8 Hz, 1H), 4.17 (s, 2H), 3.90 (d, J = 16.4 Hz, 1H), 3.47 (d, J = 16.0 Hz, 1H). |
| 147 | MD | 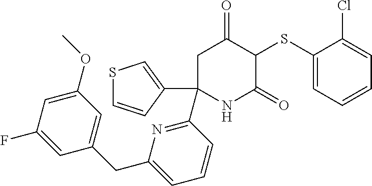 [SMILES: COc1cc(F)cc(Cc2cccc(C3(c4ccsc4)CC(=O)C(Sc4ccccc4Cl)C(=O)N3)n2)c1] | 3-((2-chlorophenyl)thio)- 6-(6-(3-fluoro-5-methoxy- benzyl)pyridin-2-yl)-6- (thiophen-3-yl)piperidine- 2,4-dione was prepared in 28% yield according to the Example 5, Step B substituting Bromomethyl- 3-fluoro-benzene for 1- (bromomethyl)-3-fluoro- 5-methoxybenzene | ¹H NMR (400 MHz, CD₃OD) δ 7.79 (dd, J = 8.0, 8.0 Hz, 1H), 7.48-7.43 (m, 2H), 7.31-7.13 (m, 4H), 6.89 (dd, J = 8.0, 8.0 Hz, 1H), 6.69-6.37 (m, 2H), 6.57 (d, J = 6.4 Hz, 1H), 6.46 (d, J = 10.8 Hz, 1H), 5.82 (dd, J = 8.0, 1.6 Hz, 1H), 4.15 (s, 2H), 4.02 (d, J = 16.8 Hz, 1H), 3.70 (s, 3H), 3.51 (d, J = 16.8 Hz, 1H). |
| 148 | SS | 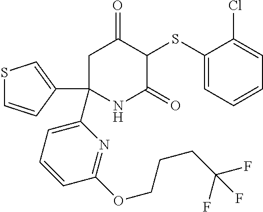 [SMILES: O=C1CC(c2ccsc2)(c2cccc(OCCCC(F)(F)F)n2)NC(=O)C1Sc1ccccc1Cl] | 3-(2-chlorophenyl)sulfanyl- 6-(3-thienyl)-6-[6-(4,4,4- trifluorobutoxy)-2- pyridyl]piperidine-2,4- dione was separated from example 121. | ¹H NMR (400 MHz, (CD₃)₂SO) δ 7.80 (dd, J = 8.0, 8.0 Hz, 1H), 7.52 (d, J = 4.8 Hz, 1H), 7.36 (dd, J = 1.2, 1.2 Hz, 1H), 7.30- 7.26 (m, 2H), 7.19 (d, J = 4.8 Hz, 1H), 6.96 (dd, J = 8.0, 8.0 Hz, 1H), 6.80 (d, J = 8.0 Hz, 1H), 6.74 (dd, J = 8.0, 8.0 Hz, 1H), 5.89 (d, J = 8.0 Hz, 1H), 4.39 (t, J = 8.0 Hz, 2H), 3.72 (d, J = 16.0 Hz, 1H), 3.27 (d, J = 16.0 Hz, 1H), 2.38-2.28 (m, 2H), 1.93- 1.88 (m, 2H). |
| 149 | SS | 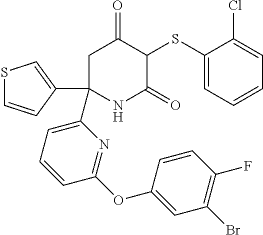 [SMILES: O=C1CC(c2ccsc2)(c2cccc(Oc3ccc(F)c(Br)c3)n2)NC(=O)C1Sc1ccccc1Cl] | 6-[6-(3-bromo-4-fluoro- phenoxy)-2-pyridyl]-3- (2-chlorophenyl)sulfanyl- 6-(3-thienyl)piperidine- 2,4-dione was prepared in 6.8% yield according to the Example 3, Step A substituting 2-Chloro-4- fluoro-phenol for 3-bromo- 4-fluorophenol | ¹H NMR (400 MHz, CD₃OD) δ 7.86 (dd, J = 8.0. 8.0 Hz, 1H), 7.39-7.33 (m, 3H), 7.25-7.15 (m, 3H), 7.10-7.06 (m, 1H), 7.03 (d, J = 4.0 Hz, 1H), 6.97 (d, J = 8.0 Hz, 1H), 6.87 (dd, J = 8.0, 8.0 Hz, 1H), 6.75 (dd, J = 8.0, 8.0 Hz, 1H), 6.08 (d, J = 8.0 Hz, 1H), 3.44 (d, J = 16.0 Hz, 1H), 3.28 (d, J = 16.0 Hz, 1H). |
| 150 | SS | 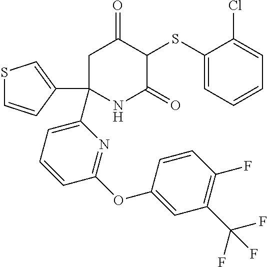 [SMILES: O=C1CC(c2ccsc2)(c2cccc(Oc3ccc(F)c(C(F)(F)F)c3)n2)NC(=O)C1Sc1ccccc1Cl] | 3-(2-chlorophenyl)sulfanyl- 6-[6-[4-fluoro-3- (trifluoromethyl)phenoxy]- 2-pyridyl]-6-(3-thienyl) piperidine-2,4-dione was prepared in 15% yield according to the Example 3, Step A substituting 2- chloro-4-fluoro-phenol for 4-fluoro-3-(trifluoro- methyl)phenol. | ¹H NMR (400 MHz, CD₃OD) δ 7.93 (dd, J = 7.6, 7.6 Hz, 1H), 7.40-7.37 (m, 5H), 7.34 (d, J = 2.8 Hz, 1H), 7.32 (d, J = 2.8 Hz, 1H), 7.17 (d, J = 7.6 Hz, 1H), 7.16-7.07 (m, 2H), 6.80 (d, J = 7.6 Hz, 1H), 5.99 (d, J = 8.0 Hz, 1H), 3.56 (d, J = 16 Hz, 1H), 3.28 (d, J = 16 Hz, 1H). |
| 151 | SS | 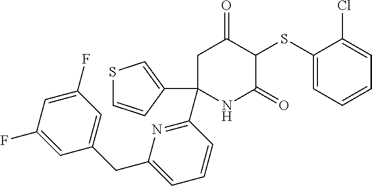 [SMILES: O=C1CC(c2ccsc2)(c2cccc(Cc3cc(F)cc(F)c3)n2)NC(=O)C1Sc1ccccc1Cl] | 3-(2-chlorophenyl)sulfanyl- 6-[6-[(3,5-difluorophenyl) methyl]-2-pyridyl]-6-(3- thienyl)piperidine-2,4- dione was prepared according to methods described therein | ¹H NMR (400 MHz, CD₃OD) δ 7.76 (dd, J = 8.0. 8.0 Hz, 1H), 7.46 (d, J = 8.0 Hz, 1H), 7.42 (dd, J = 0.8, 0.4 Hz, 1H), 7.28-7.23 (m, 2H), 7.15 (dd, J = 8.0, 8.0 Hz, 1H), 7.08 (dd, J = 5.6, 0.8 Hz, 1H), 6.89-6.84 (m, 3H), 6.68 (dd, J = 8.0, 8.0 Hz, 1H), 6.56 (dd, J = 8.0, 8.0 Hz, 1H), 5.85 (d, J = 8.4 Hz, 1H), 4.16 (s, 2H), 3.83 (d, J = 16.4 Hz, 1H), 3.45 (d, J = 16.0 Hz, 1H). |
| 152 | SS | 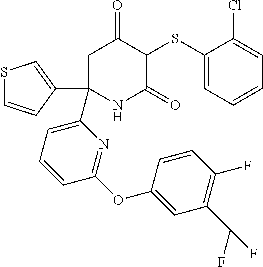 [SMILES: O=C1CC(c2ccsc2)(c2cccc(Oc3ccc(F)c(C(F)F)c3)n2)NC(=O)C1Sc1ccccc1Cl] | 3-(2-chlorophenyl)sulfanyl- 6-[6-[3-(difluoromethyl)- 4-fluoro-phenoxy]-2- pyridyl]-6-(3-thienyl) piperidine-2,4-dione was prepared in 7.2% yield according to the Example 3, Step A substituting 2-Chloro-4-fluoro-phenol for 4-(difluoromethyl)-3- fluorophenol | ¹H NMR (400 MHz, CD₃OD) δ 7.86 (dd, J = 8.0, 8.0 Hz, 1H), 7.34 (dd, J = 8.0, 8.0 Hz, 2H), 7.26-7.21 (m, 3H), 7.20 (s, 1H), 7.14 (d, J = 8.0 Hz, 1H), 6.98- 6.92 (m, 3H), 6.84 (dd, J = 8.0, 8.0 Hz, 1H), 6.73 (dd, J = 8.0, 8.0 Hz, 1H), 6.09 (d, J = 8.0 Hz, 1H), 3.39 (d, J = 16.0 Hz, 1H), 3.25 (d, J = 16.0 Hz, 1H). |
| 153 | SS | 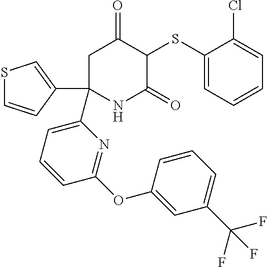 [SMILES: O=C1CC(c2ccsc2)(c2cccc(Oc3cccc(C(F)(F)F)c3)n2)NC(=O)C1Sc1ccccc1Cl] | 3-(2-chlorophenyl)sulfanyl- 6-(3-thienyl)-6-[6-[3- (trifluoromethyl)phenoxy]- 2-pyridyl]piperidine-2,4- dione was separated from example 140. | ¹H NMR (400 MHz, (CD₃)₂SO) δ 7.99 (dd, J = 8.0, 8.0 Hz, 1H), 7.66-7.58 (1H, 2H), 7.48-7.40 (m, 4H), 7.30-7.25 (m, 2H), 7.07 (d, J = 8.0 Hz, 1H), 7.01 (d, J = 4.0 Hz, 1H), 6.96 (dd, J = 8.0, 8.0 Hz, 1H), 6.79 (dd, J = 8.0, 8.0 Hz, 1H), 5.95 (d, J = 8.0 Hz, 1H), 3.38 (d, J = 16.0 Hz, 1H), 3.21 (d, J = 16.0 Hz, 1H). |
| 154 | MD | 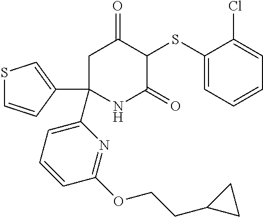 [SMILES: O=C1CC(c2ccsc2)(c2cccc(OCCC3CC3)n2)NC(=O)C1Sc1ccccc1Cl] | 3-(2-chlorophenoxy)-6- (6-(2-cyclopropylethoxy) pyridin-2-yl)-6-(thiophen- 3-yl)piperidine-2,4- dione was prepared in 32% yield according to the Example 2, Step A substituting propan-2-ol for 2-cyclopropylethanol | ¹H NMR (400 MHz, CD₃OD) δ 7.75 (dd, J = 8.0, 8.0 Hz, 1H), 7.44 (dd, J = 8.0, 4.0 Hz, 1H), 7.30-7.29 (m, 2H), 7.19-7.16 (m, 2H), 6.87-6.85 (m, 2H), 6.76 (d, J =8.0 Hz, 1H), 6.01 (dd, J = 8.0, 4.0 Hz, 1H), 4.50-4.42 (1H, 2H), 3.82 (d, J = 16.0 Hz, 1H), 3.33 (d, J = 16.0 Hz, 1H), 1.69- 1.64 (m, 2H), 0.85-0.83 (m, 1H), 0.46-0.43 (m, 2H), 0.12-0.08 (m, 2H). |
| 155 | SS | 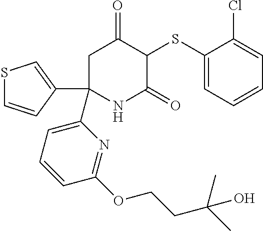 [SMILES: CC(C)(O)CCOc1cccc(C2(c3ccsc3)CC(=O)C(Sc3ccccc3Cl)C(=O)N2)n1] | 3-(2-chlorophenyl)sulfanyl- 6-[6-(3-hydroxy-3- methyl-butoxy)-2-pyridyl]- 6-(3-thienyl)piperidine- 2,4-dione was prepared in 13% yield according to the Example 2, Step A substituting propan-2-ol for 3-methylbutane- 1,3-diol. | ¹H NMR (400 MHz, CD₃OD) δ 7.72 (dd, J = 7.2, 7.2 Hz, 1H), 7.43 (dd, J = 4.8, 2.8 Hz, 1H), 7.30 (dd, J = 2.8, 1.2 Hz, 1H), 7.20 (dd, J = 2.8, 1.6 Hz, 1H), 7.18 (d, J = 1.6 Hz, 1H), 7.17 (d, J = 7.2 Hz, 1H), 6.92-6.88 (m, 1H), 6.75-6.72 (m, 2H), 6.05 (dd, J = 8.0, 1.6 Hz, 1H), 4.55-4.50 (m, 1H), 3.79 (d, J = 16.0 Hz, 1H), 3.42 (d, J = 16.0 Hz, 1H), 1.92 (t, J = 7.2Hz, 2H), 1.25 (d, J = 2.8 Hz, 6H). |
| 156 | SS | 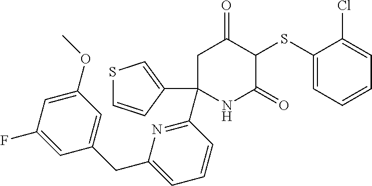 [SMILES: COc1cc(F)cc(Cc2cccc(C3(c4ccsc4)CC(=O)C(Sc4ccccc4Cl)C(=O)N3)n2)c1] | 3-(2-chlorophenyl)sulfanyl- 6-[6-[(3-fluoro-5- methoxy-phenyl)methyl]- 2-pyridyl]-6-(3-thienyl) piperidine-2,4-dione was prepared according to methods described therein | ¹H NMR (400 MHz, CD₃OD) δ 7.77 (dd, J = 8.0, 8.0 Hz, 1H), 7.48-7.42 (m, 2H), 7.30-7.13 (m, 4H), 6.68-6.57 (m, 3H), 6.47 (d, J = 6.4 Hz, 1H), 6.46 (d, J = 11.2 Hz, 1H), 5.85 (d, J = 8.0 Hz, 1H), 4.15 (s, 2H), 3.97 (d, J = 16.4 Hz, 1H), 3.70 (s, 3H), 3.50 (d, J = 16.0 Hz, 1H). |
| 157 | MD | 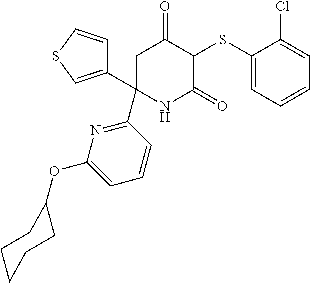 [SMILES: O=C1CC(c2ccsc2)(c2cccc(OC3CCCCC3)n2)NC(=O)C1Sc1ccccc1Cl] | 3-(2-chlorophenyl)sulfanyl- 6-[6-(cyclohexoxy)-2- pyridyl]-6-(3-thienyl) piperidine-2,4-dione was prepared in 13.2% yield according to the Example 2, Step A substituting propan-2-ol for cyclohexanol. | ¹H NMR (400 MHz, (CD₃)₂SO) δ 8.45 (s, 1H), 7.75 (dd, J = 8.0, 8.0 Hz, 1H), 7.52 (dd, J = 2.0. 2.0 Hz, 1H), 7.36 (dd, J = 1.4, 1.4 Hz, 1H), 7.28 (dd, J = 4.0, 4.0 Hz, 1H), 7.21-7.17 (m, 2H), 6.97 (dd, J = 4.0, 4.0 Hz, 1H), 6.73-6.70 (m, 2H), 5.87 (d, J = 8.0 Hz, 1H), 5.03-4.97 (m, 1H), 3.83 (d, J = 16.0 Hz, 1H), 3.36 (d, J = 16.0 Hz, 1H), 1.89-1.87 (m,2H), 1.70-1.67 (m, 2H), 1.55-1.24 (m, 6H). |
| 158 | MD | 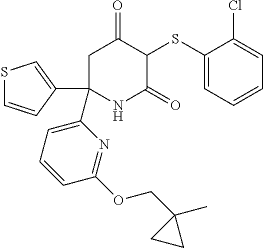 [SMILES: CC1(COc2cccc(C3(c4ccsc4)CC(=O)C(Sc4ccccc4Cl)C(=O)N3)n2)CC1] | 3-(2-chlorophenyl)sulfanyl- 6-[6-[(1-methylcyclo- propyl)methoxy]-2-pyridyl]- 6-(3-thienyl)piperidine-2,4- dione was prepared in 7% yield according to the Example 2, Step A substituting propan-2-ol for (1-methylcyclopropyl) methanol. | ¹H NMR (400 MHz, CD₃OD) δ 7.74 (dd, J = 7.6, 7.6 Hz, 1H), 7.46 (dd, J = 5.2, 2.8 Hz, 1H), 7.29 (dd, J = 2.8, 1.6 Hz, 1H), 7.24 (dd, J = 8.0, 1.2 Hz, 1H), 7.17-7.15 (m, 2H), 6.98-6.94 (m, 1H), 6.81 (d, J = 8.4 Hz, 1H), 6.80-6.76 (m, 1H), 5.98 (dd, J = 7.6, 1.2 Hz, 1H), 4.17 (d, J = 1.6 Hz, 2H), 3.89 (d, J = 16.4 Hz, 1H), 3.47 (d, J = 16.4 Hz, 1H), 1.19 (s, 3H), 0.57-0.49 (m, 2H), 0.41-0.34 (m, 2H). |
| 159 | MD | 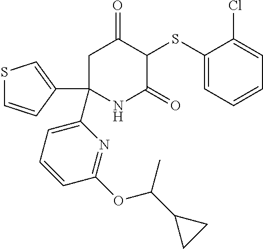 [SMILES: CC(Oc1cccc(C2(c3ccsc3)CC(=O)C(Sc3ccccc3Cl)C(=O)N2)n1)C1CC1] | 3-((2-chlorophenyl)thio)- 6-(6-(1-cyclopropyl- ethoxy)pyridin-2-yl)-6- (thiophen-3-yl)piperidine- 2,4-dione was prepared in 32% yield according to the Example 2, Step A substituting propan-2-ol for 1-cyclopropylethanol | ¹H NMR (400 MHz, CD₃OD) δ 7.69 (dd, J = 8.0, 1.6 Hz, 1H), 7.43 (dd, J = 5.2, 3.2 Hz, 1H), 7.28-7.16 (m, 2H), 7.15-7.06 (m, 2H), 6.93 (dd, J = 4.0, 4.0 Hz, 1H), 6.71-6.69 (m, 2H), 5.95 (dd, J = 8.0, 6.8 Hz, 1H), 4.79-4.73 (m, 1H), 3.83 (dd, J = 16.0, 2.4 Hz, 1H), 3.49- 3.41 (m, 1H), 1.36-1.28 (m, 3H), 1.10-1.07 (m, 1H), 0.46- 0.16 (m, 4H). |
| 160 | MD | 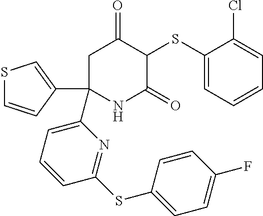 [SMILES: O=C1CC(c2ccsc2)(c2cccc(Sc3ccc(F)cc3)n2)NC(=O)C1Sc1ccccc1Cl] | 3-(2-chlorophenyl)sulfanyl- 6-[6-(4-fluorophenyl) sulfanyl-2-pyridyl]-6-(3- thienyl)piperidine-2,4- dione was prepared in 14% yield according to the Example 2, Step A substituting propan-2-ol for 4-fluorobenzenethiol. | ¹H NMR (400 MHz, CD₃OD) δ 7.63 (dd, J = 8.0, 8.0 Hz, 1H), 7.61-7.56 (m, 2H), 7.39 (dd, J = 5.2, 3.0 Hz, 1H), 7.29 (dd, J = 7.7, 0.7 Hz, 1H), 7.23-7.17 (m, 4H), 7.04 (dd, J = 7.9, 0.7 Hz, 1H), 6.98 (dd, J = 5.1, 1.3 Hz, 1H), 6.97-6.92 (m, 1H), 6.79-6.75 (m, 1H), 5.98 (dd, J = 8.0, 1.4 Hz, 1H), 3.70 (d, J = 16.5 Hz, 1H), 3.34 (d, J = 16.5 Hz, 1H). |
| 161 | MD | 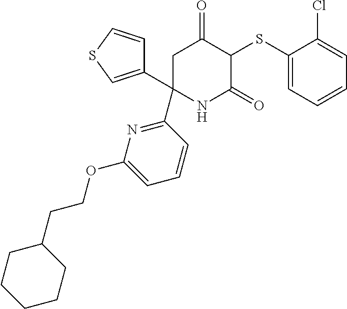 [SMILES: O=C1CC(c2ccsc2)(c2cccc(OCCC3CCCCC3)n2)NC(=O)C1Sc1ccccc1Cl] | 3-(2-chlorophenyl)sulfanyl- 6-[6-(2-cyclohexylethoxy)- 2-pyridyl]-6-(3-thienyl) piperidine-2,4-dione was prepared in 7.4% yield according to the Example 2, Step A substituting propan-2-ol for 2- cyclohexylethanol | ¹H NMR (400 MHz, CD₃OD) δ 8.48 (s, 1H), 7.74 (dd, J = 8.0, 8.0 Hz, 1H), 7.49 (dd, J = 4.0, 4.0 Hz, 1H), 7.32 (s, 1H), 7.27 (d, J = 8.0 Hz, 1H), 7.19 (d, J = 8.0 Hz, 1H), 7.13 (d, J = 4.0 Hz, 1H), 6.94 (dd, J = 8.0, 8.0 Hz, 2H),5.80 (d, J = 8.0 Hz, 1H), 4.32-4.26 (m,2H), 3.90 (d, J = 16.0 Hz, 1H), 3.34 (d, J = 16.0 Hz, 1H), 1.69-1.59 (m, 7H), 1.55-1.50 (m, 1H), 1.13-1.08 (m, 3H), 0.91-0.83 (m, 2H). |
| 162 | MD | 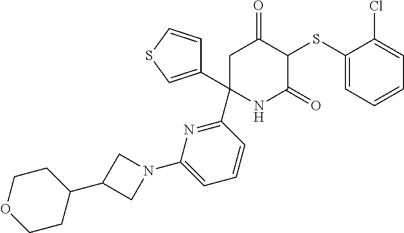 [SMILES: O=C1CC(c2ccsc2)(c2cccc(N3CC(C4CCOCC4)C3)n2)NC(=O)C1Sc1ccccc1Cl] | 3-(2-chlorophenyl)sulfanyl- 6-[6-(3-tetrahydropyran- 4-ylazetidin-1-yl)-2- pyridyl]-6-(3-thienyl) piperidine-2,4-dione was prepared in 10% yield according to the Example 4, Step A substituting cyclohexanamine for 3- (tetrahydro-2H-pyran-4- yl)azetidine. | ¹H NMR (400 MHz, CD₃OD) δ 7.65 (dd, J = 7.6, 7.6 Hz, 1H), 7.48 (d, J = 2.8 Hz, 1H), 7.35 (d, J = 2.8 Hz, 1H), 7.24 (d, J = 8.0 Hz, 1H), 6.97 (d, J = 7.6 Hz, 1H), 6.79 (d, J = 7.4 Hz, 1H), 6.70 (d, J = 7.2 Hz, 1H), 6.50 (d, J = 7.2 Hz, 1H), 6.0 (d, J = 8.0 Hz, 1H), 4.16- 3.93 (m, 2H), 3.91-3.78 (m, 5H), 3.45-3.34 (m, 3H), 2.51- 2.49 (m, 1H), 1.59-1.56 (m, 3H), 1.22-1.14 (m,2H). |
| 163 | MD | 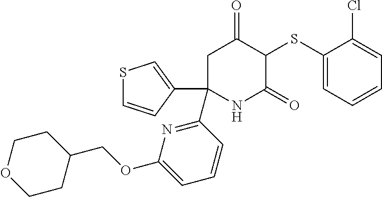 [SMILES: O=C1CC(c2ccsc2)(c2cccc(OCC3CCOCC3)n2)NC(=O)C1Sc1ccccc1Cl] | 3-((2-chlorophenyl)thio)-6- (6-((tetrahydro-2H-pyran- 4-yl)methoxy)pyridin- 2-yl)-6-(thiophen-3- yl)piperidine-2,4-dione was prepared in 36% yield according to the Example 2, Step A substituting propan-2-ol for (tetrahydro- 2H-pyran-4-yl)methanol | ¹H NMR (400 MHz, CD₃OD) δ 7.72 (dd, J = 8.0, 8.0 Hz, 1H), 7.43 (dd, J = 5.2, 3.2 Hz, 1H), 7.25-7.13 (m, 4H), 6.77 (dd, J = 7.6, 7.6 Hz, 1H), 6.73- 6.69 (m, 2H), 5.90 (dd, J = 8.0, 1.6 Hz, 1H), 4.31-4.29 (m, 1H), 4.15-4.11 (m, 1H), 3.92-3.81 (m, 3H), 3.46-3.25 (m, 3H), 2.14-1.97 (m, 1H), 1.97-1.63 (m, 2H), 1.39-1.31 (m, 2H). |
| 164 | MD | 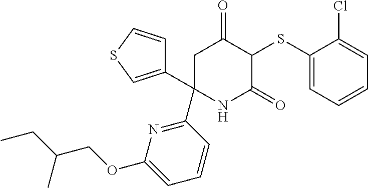 [SMILES: CCC(C)COc1cccc(C2(c3ccsc3)CC(=O)C(Sc3ccccc3Cl)C(=O)N2)n1] | 3-(2-chlorophenyl)sulfanyl- 6-[6-(2-methylbutoxy)-2- pyridyl]-6-(3-thienyl) piperidine-2,4-dione was prepared in 9.7% yield according to the Example 2, Step A substituting propan-2-ol for (S)-2- methylbutan-1-ol. | ¹H NMR (400MHz, (CD₃)₂SO) δ 8.34 (s, 1H) 7.73 (dd, J = 8.0, 8.0 Hz 1H), 7.48 (dd, J = 4.8, 4.8 Hz, 1H), 7.32 (d, J = 1.6 Hz, 1H), 7.24 (d, J = 8.0 Hz, 1H), 7.18 (d, J = 7.6 Hz, 1H), 7.13 (d, J = 5.2 Hz, 1H), 6.92 (dd, J = 8.0, 8.0 Hz, 1H), 6.74- 6.70 (m, 2H), 5.84 (d, J = 8.0 Hz, 1H), 4.16- 4.05 (m, 2H), 3.82 (d, J = 16.0 Hz, 1H), 3.35 (d, J = 16.0 Hz, 1H), 1.76-1.71 (m, 1H), 1.46- |
| | | | | 1.43 (m, 1H), 1.17-1.14 |
| | | | | (m, 1H), 0.90-0.81 (m, 6H). |
| 165 | MD | 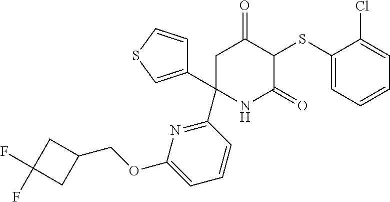 [SMILES: O=C1CC(c2ccsc2)(c2cccc(OCC3CC(F)(F)C3)n2)NC(=O)C1Sc1ccccc1Cl] | 3-(2-chlorophenyl)sulfanyl- 6-[6-[(3,3-difluoro- cyclobutyl(methoxy]-2- pyridyl]-6-(3-thienyl) piperidine-2,4-dione was prepared in 7.3% yield according to the Example 2, Step A substituting propan-2-ol for (3,3- difluorocyclobutyl)methanol | ¹H NMR (400 MHz, (CD₃)₂SO) δ 8.52 (s, 1H), 7.77 (dd, J = 8.0, 8.0 Hz, 1H), 7.50 (dd, J = 4.0, 4.0 Hz, 1H), 7.34 (d, J = 4.0 Hz, 1H), 7.28-7.21 (m, 2H), 7.15 (d, J = 4.0 Hz, 1H), 6.94 (dd, J = 8.0, 8.0 Hz, 1H), 6.77 (d, J = 8.0 Hz, 1H), 6.70 (dd, J = 8.0, 8.0 Hz, 1H), 5.79 (d, J = 8.0 Hz, 1H), 4.33 (d, J = 8.0 Hz, 2H), 3.90 (d, J = 16.0 Hz, 1H), 3.36 (d, J = 16.0 Hz, 1H), 3.26 (d, J =16.0 Hz, 1H) 2.65- 2.57 (m, 2H), 2.42-2.36 (m, 2H). |
| 166 | MD | 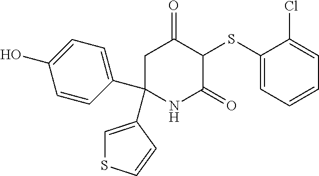 [SMILES: O=C1CC(c2ccc(O)cc2)(c2ccsc2)NC(=O)C1Sc1ccccc1Cl] | 3-(2-chlorophenyl)sulfanyl- 6-(4-hydroxyphenyl)-6- (3-thienyl)piperidine-2,4- dione was prepared in 38% yield according to Example 7, Step H substituting 2- methylmorpholine for sodium hydroxide. | ¹H NMR (400 MHz, (CD₃)₂SO) δ 9.44 (s, 1H), 8.32 (s, 1H), 7.55- 7.54 (m, 1H), 7.28-7.25 (m, 2H), 7.17-7.11 (m, 3H), 6.94 (dd, J = 8.0, 8.0 Hz, 1H), 6.73- 6.71 (m, 3H), 5.84 (d, J = 8.0 Hz, 1H), 3.30 (s, 2H). |
| 167 | SS | 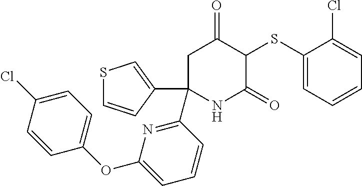 [SMILES: O=C1CC(c2ccsc2)(c2cccc(Oc3ccc(Cl)cc3)n2)NC(=O)C1Sc1ccccc1Cl] | 6-[6-(4-chlorophenoxy)-2- pyridyl]-3-(2-chlorophenyl) sulfanyl-6-(3-thienyl) piperidine-2,4-dione was prepared according to methods described therein | ¹H NMR (400 MHz, CD₃OD) δ 7.91 (dd. J = 7.9, 7.9 Hz, 1H), 7.42 (dd, J = 5.2, 3.2 Hz, 1H), 7.40-7.37 (m, 3H), 7.25 (dd, J = 8.0, 1.2 Hz, 1H), 7.21 (dd, J = 3.2, 1.2 Hz, 1H), 7.10-7.03 (m, 4H), 7.00-6.94 (m, 1H), 6.85- 6.78 (m, 1H), 6.04 (dd, J = 8.0, 1.2 Hz, 1H), 3.64 (d, J = 16.3 Hz, 1H), 3.34 (d, J = 16.3 Hz, 1H). |
| 168 | SS | 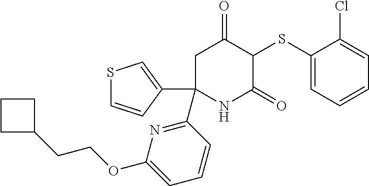 [SMILES: O=C1CC(c2ccsc2)(c2cccc(OCCC3CCC3)n2)NC(=O)C1Sc1ccccc1Cl] | 3-(2-chlorophenyl)sulfanyl- 6-[6-(2-cyclobutylethoxy)- 2-pyridyl]-6-(3-thienyl) piperidine-2,4-dione was prepared according to methods described therein | ¹H NMR (400 MHz, CD₃OD) δ 7.71 (dd, J = 8.4, 4.0 Hz, 1H), 7.46 (dd, J = 5.2, 1.2 Hz, 1H), 7.30-7.20 (m, 2H), 7.15-7.12 (m, 2H), 6.93 (dd, J = 8.0, 4.0 Hz, 1H), 6.73-6.71 (m, 2H), 6.05 (dd, J = 8.0, 1.6 Hz, 1H), 4.34-4.29 (m, 2H), 3.92 (d, J = 16.0 Hz, 1H), 3.47 (d, J = 16.0 Hz, 1H), 2.49-2.39 (m, 1H), 2.09-1.67 (m, 8H). |
| 169 | SS | 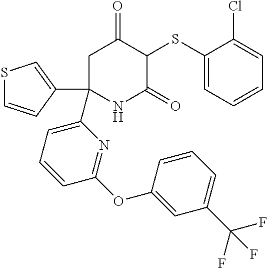 [SMILES: O=C1CC(c2ccsc2)(c2cccc(Oc3cccc(C(F)(F)F)c3)n2)NC(=O)C1Sc1ccccc1Cl] | 3-(2-chlorophenyl)sulfanyl- 6-(3-thienyl)-6-[6-[3- (trifluoromethyl)phenoxy]- 2-pyridyl]piperidine-2,4- dione was separated from example 140. | ¹H NMR (400 MHz, (CD₃)₂SO) δ 7.99 (dd, J = 8.0, 8.0 Hz, 1H), 7.66-7.58 (m, 2H), 7.48-7.40 (m, 4H), 7.30-7.25 (m, 2H), 7.07 (d, J = 8.0 Hz, 1H), 7.01 (d, J = 4.0 Hz, 1H), 6.96 (dd, J = 8.0, 8.0 Hz, 1H), 6.79 (dd, J = 8.0, 8.0 Hz, 1H), 5.95 (d, J = 8.0 Hz, 1H), 3.46 (d, J = 16.0 Hz, 1H), 3.19 (d, J = 16.0 Hz, 1H). |
| 170 | SS | 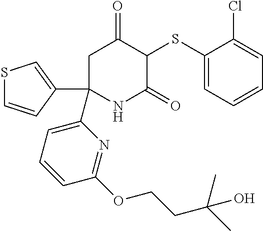 [SMILES: CC(C)(O)CCOc1cccc(C2(c3ccsc3)CC(=O)C(Sc3ccccc3Cl)C(=O)N2)n1] | 3-(2-chlorophenyl)sulfanyl- 6-[6-(3-hydroxy-3-methyl- butoxy)-2-pyridyl]-6-(3- thienyl)piperidine-2,4- dione was prepared in 13% yield according to the Example 2, Step A substituting propan-2-ol for 3-methylbutane-1,3- diol. | ¹H NMR (400 MHz, CD₃OD) δ 7.71 (dd, J = 8.3, 7.5 Hz, 1H), 7.43 (dd, J = 5.0, 3.0 Hz, 1H), 7.30 (dd, J = 3.0, 1.5 Hz, 1H), 7.20 (dd, J = 3.6, 1.6 Hz, 1H), 7.18 (d, J = 1.6 Hz, 1H), 7.17 (d, J = 6.8 Hz, 1H), 6.92-6.88 m, 1H), 6.76-6.72 (m, 2H), 6.04 (dd, J = 8.0, 1.6 Hz, 1H), 4.55-4.51 (m, 2H), 3.79 (d, J = 16.4 Hz, 1H), 3.42 (d, J = 16.1 Hz, 1H), 1.92 (t, J = 7.2 Hz, 2H), 1.25 (d, J = 3.3 Hz, 6H). |
| 171 | SS | 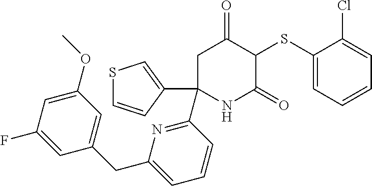 [SMILES: COc1cc(F)cc(Cc2cccc(C3(c4ccsc4)CC(=O)C(Sc4ccccc4Cl)C(=O)N3)n2)c1] | 3-(2-chlorophenyl)sulfanyl- 6-[6-[(3-fluoro-5- methoxy-phenyl)methyl]- 2-pyridyl]-6-(3-thienyl) piperidine-2,4-dione was prepared according to methods described therein | ¹H NMR (400 MHz, CD₃OD) δ 7.77 (dd, J = 8.0, 8.0 Hz, 1H), 7.48-7.42 (m, 2H), 7.30-7.13 (m, 4H), 6.68-6.57 (m, 3H), 6.47 (d, J = 6.4 Hz, 1H), 6.46 (d, J = 11.2 Hz, 1H), 5.85 (d, J = 8.0 Hz, 1H), 4.15 (s, 2H), 3.96 (d, J = 16.4 Hz, 1H), 3.70 (s, 3H), 3.50 (d, J = 16.0 Hz, 1H). |
| 172 | SS | 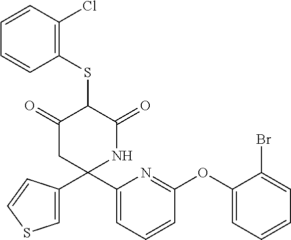 [SMILES: O=C1CC(c2ccsc2)(c2cccc(Oc3ccccc3Br)n2)NC(=O)C1Sc1ccccc1Cl] | 6-[6-(2-bromophenoxy)- 2-pyridyl]-3-(2-chloro- phenyl)sulfanyl-6-(3- thienyl)piperidine-2,4-dione was prepared in 6% yield according to the Example 3, Step A substituting 2-chloro-4-fluoro-phenol for 2-bromophenol. | ¹H NMR (400 MHz, CD₃OD) δ 7.88 (dd. J = 7.6, 7.6 Hz, 1H), 7.67 (d, J = 6.8 Hz, 1H), 7.39- 7.30 (m, 3H), 7.15-7.13 (m, 4H), 7.03 (d, J = 2.8 Hz, 1H), 6.96 (d, J = 2.8 Hz, 1H), 6.79 (d, J = 7.6 Hz, 1H), 5.99 (d, J = 8.0 Hz, 1H), 3.51 (d, J = 16.0 Hz, 1H), 3.21 (d, J = 16.0 Hz, 1H). |
| 173 | MD | 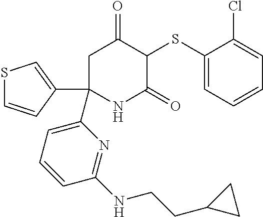 [SMILES: O=C1CC(c2ccsc2)(c2cccc(NCCC3CC3)n2)NC(=O)C1Sc1ccccc1Cl] | 3-(2-chlorophenyl)sulfanyl- 6-[6-(2-cyclopropyl- ethylamino)-2-pyridyl]-6- (3-thienyl)piperidine-2,4- dione was prepared according to methods described therein. | |
| 174 | MD | 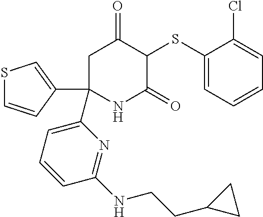 [SMILES: O=C1CC(c2ccsc2)(c2cccc(NCCC3CC3)n2)NC(=O)C1Sc1ccccc1Cl] | 3-(2-chlorophenyl)sulfanyl- 6-[6-(2-cyclopropyl- ethylamino)-2-pyridyl]-6- (3-thienyl)piperidine-2,4- dione was prepared according to methods described therein. | |
| 175 | SS | 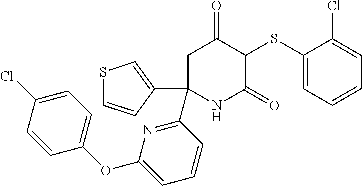 [SMILES: O=C1CC(c2ccsc2)(c2cccc(Oc3ccc(Cl)cc3)n2)NC(=O)C1Sc1ccccc1Cl] | 6-[6-(4-chlorophenoxy)-2- pyridyl]-3-(2-chlorophenyl) sulfanyl-6-(3-thienyl) piperidine-2,4- dione was prepared according to methods described therein. | ¹H NMR (400 MHz, CD₃OD) δ 7.88 (dd, J = 7.9, 7.9 Hz, 1H), 7.39 (dd, J = 5.2, 3.2 Hz, 1H), 7.41-7.37 (m, 3H), 7.22 (dd, J = 8.0, 1.2 Hz, 1H), 7.17 (dd, J = 3.2, 1.2 Hz, 1H), 7.07-6.98 (m, 4H), 6.97-6.92 (m, 1H), 6.82-6.75 (m, 1H), 6.01 (dd, J = 8.0, 1.2 Hz, 1H), 3.61 (d, J = 16.3 Hz, 1H), 3.30 (d, J = 16.3 Hz, 1H). |
| 176 | MD | 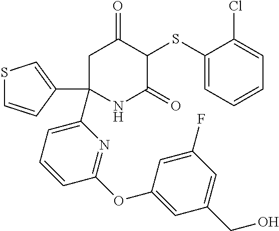 [SMILES: O=C1CC(c2ccsc2)(c2cccc(Oc3cc(F)cc(CO)c3)n2)NC(=O)C1Sc1ccccc1Cl] | 3-(2-chlorophenyl)sulfanyl- 6-[6-[3-fluoro-5- (hydroxymethyl)phenoxy]- 2-pyridyl]-6-(3-thienyl) piperidine-2,4-dione was prepared in 7.2% yield according to the Example 3, Step A substituting 2-Chloro-4-fluoro-phenol for 3-fluoro-5-(hydroxy- methyl)phenol | ¹H NMR (400 MHz, CD₃OD) δ 8.50 (s, 1H), 7.96 (dd, J = 8.0, 8.0 Hz, 1H), 7.49-7.43 (m, 2H), 7.30-7.25 (m, 2H), 7.06-6.95 (m, 4H), 6.79-6.73 (m, 3H), 5.90 (d, J = 8.0 Hz, 1H), 5.37 (s, 1H), 4.45 (d, J = 7.6 Hz, 2H), 3.61 (d, J = 16.0 Hz, 1H), 3.23 (d, J = 16.0 Hz, 1H). |
| 177 | MD | 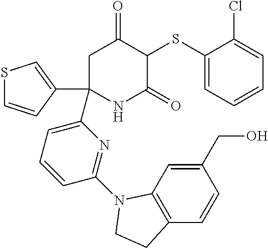 [SMILES: O=C1CC(c2ccsc2)(c2cccc(N3CCc4ccc(CO)cc43)n2)NC(=O)C1Sc1ccccc1Cl] | 3-(2-chlorophenyl)sulfanyl- 6-[6-[6-(hydroxymethyl) indolin-1-yl]-2-pyridyl]-6- (3-thienyl)piperidine- 2,4-dione was prepared in 3.5% yield according to Example 3, Step A substituting 2-Chloro-4- fluoro-phenol for indolin- 6-ylmethanol. | ¹H NMR (400 MHz, (CD₃)₂SO) δ 8.48 (s, 1H), 8.12 (s, 1H), 7.78 (dd, J = 7.6, 7.6 Hz, 1H), 7.49- 7.47 (m, 2H), 7.26-7.23 (m, 2H), 7.12-7.09 (m, 2H), 6.83 (d, J = 8.4 Hz, 1H), 6.79-6.77 (m, 3H), 5.99 (d, J = 8.0 Hz, 1H), 4.50-4.49 (m, 2H), 4.04 (dd, J = 8.0, 8.0 Hz, 2H), 3.96 (d, J = 16.8 Hz, 1H), 3.50 (d, J = 16.4 Hz, 1H), 3.12 (t, J = 8.0 Hz, 2H). |
| 178 | SS | 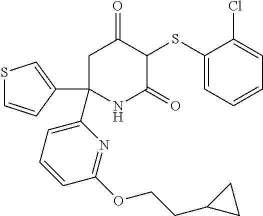 [SMILES: O=C1CC(c2ccsc2)(c2cccc(OCCC3CC3)n2)NC(=O)C1Sc1ccccc1Cl] | 3-(2-chlorophenoxy)-6-[6- (2-cyclopropylethoxy)-2- pyridyl]-6-(3-thienyl) piperidine-2,4-dione was prepared according to methods described therein | ¹H NMR (400 MHz, CD₃OD) δ 7.65 (dd, J = 8.0, 8.0 Hz, 1H), 7.36 (dd, J = 8.0, 4.0 Hz, 1H), 7.26-7.23 (m, 2H), 7.10-7.08 (m, 2H), 6.79-6.76 (m, 2H), 6.69 (d, J = 8.0 Hz, 1H), 5.94 (dd, J = 8.0, 4.0 Hz, 1H), 4.41-4.34 (m, 2H), 3.74 (d, J = 16.0 Hz, 1H), 3.35 (d, J = 16.0 Hz, 1H), 1.61-1.56 (m, 2H), 0.78-0.73 (m, 1H), 0.39-0.36 (m, 2H), 0.04-0.00 (m, 2H). |
| 179 | MD | 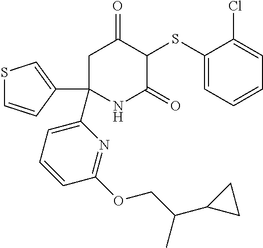 [SMILES: CC(COc1cccc(C2(c3ccsc3)CC(=O)C(Sc3ccccc3Cl)C(=O)N2)n1)C1CC1] | 3-((2-chlorophenyl)thio)-6- (6-(2-cyclopropyl- propoxy)pyridin-2-yl)- 6-(thiophen-3-yl)piperidine- 2,4-dione was prepared in 5% yield according to the Example 2, Step A substituting propan-2-ol for 2-cyclopropylpropan-1-ol | ¹H NMR (400 MHz, CD₃OD) δ 7.73 (dd, J = 8.4, 1.6 Hz, 1H), 7.45 (d, J = 2.4 Hz, 1H), 7.28- 7.14 (m, 4H), 6.95 (t, J = 4.0, 4.0 Hz, 1H), 6.78-6.76 (m, 2H), 6.02 (d, J = 8.0 Hz, 1H), 4.46-4.42 (m, 1H), 4.25-4.23 (m, 1H), 3.91 (d, J = 16.0 Hz, 1H), 3.47 (d, J = 16.0 Hz, 1H), 1.18-1.06 (m, 4H), 0.65-0.63 (m, 1H), 0.42-0.39 (m, 2H), 0.19-0.06 (m, 2H). |
| 180 | SS | 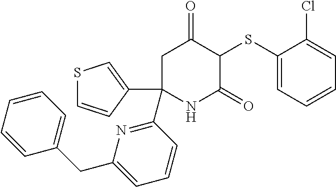 [SMILES: O=C1CC(c2ccsc2)(c2cccc(Cc3ccccc3)n2)NC(=O)C1Sc1ccccc1Cl] | 6-(6-benzyl-2-pyridyl)-3- (2-chlorophenyl)sulfanyl- 6-(3-thienyl)piperidine-2,4- dione was prepared according to methods described therein | ¹H NMR (400 MHz, (CD₃)₂SO) δ 11.66 (s, 1H), 8.27 (s, 1H), 7.74 (dd, J = 8.0, 8.0 Hz, 1H), 7.47 (d, J = 2.0 Hz, 2H), 7.31-7.10 (m, 9H), 6.89 (dd, J = 8.0, 8.0 Hz, 1H), 6.58 (dd, J = 8.0, 8.0 Hz, 1H), 5.86 (d, J = 8.0 Hz, 1H), 4.10 (s, 2H), 3.84 (d, J = 16.0 Hz, 1H), 3.33 (d, J = 16.0 Hz, 1H). |
| 181 | SS | 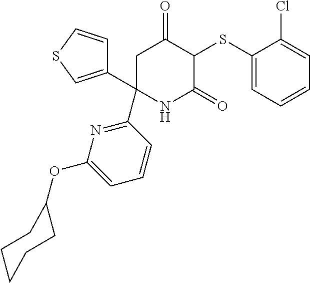 [SMILES: O=C1CC(c2ccsc2)(c2cccc(OC3CCCCC3)n2)NC(=O)C1Sc1ccccc1Cl] | 3-(2-chlorophenyl)sulfanyl- 6-[6-(cyclohexoxy)-2- pyridyl]-6-(3-thienyl) piperidine-2,4-dione was separated from example 157. | ¹H NMR (400 MHz, (CD₃)₂SO) δ 7.71 (dd, J = 8.0, 8.0 Hz, 1H), 7.48 (dd, J = 2.0, 2.0 Hz, 1H) 7.33 (dd, J = 1.4, 1.4 Hz, 1H), 7.25 (dd, J = 4.0, 4.0 Hz, 1H), 7.18-7.13 (m, 2H), 6.92 (dd, J = 4.0, 4.0 Hz, 1H), 6.70-6.65 (m, 2H), 5.86 (d, J = 8.0 Hz, 1H),5.00-4.95 (m, 1H), 3.76 (d, J= 16.0 H, 1H), 3.33 (d, J = 16.0 Hz 1H), 1.85-1.49 (m, 4H), 1.37-1.21 (m, 6H). |
| 182 | SS | 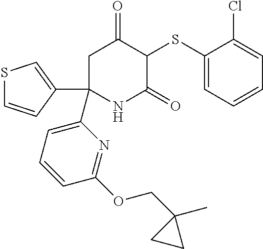 [SMILES: CC1(COc2cccc(C3(c4ccsc4)CC(=O)C(Sc4ccccc4Cl)C(=O)N3)n2)CC1] | 3-(2-chlorophenyl)sulfanyl- 6-[6-[(1-methylcyclo- propyl)methoxy]-2-pyridyl]- 6-(3-thienyl)piperidine- 2,4-dione was prepared in 7% yield according to the Example 2, Step A substituting propan-2-ol for (1-methylcyclopropyl) methanol. | ¹H NMR (400 MHz, CD₃OD) δ 7.70 (dd, J = 7.6, 7.6 Hz, 1H), 7.41 (dd, J = 5.2, 2.8 Hz, 1H), 7.27 (dd, J = 2.8, 1.6 Hz, 1H), 7.19 (dd, J = 8.0, 1.2 Hz, 1H), 7.14-7.11 (m, 2H), 6.92-6.88 (m, 1H), 6.76 (d, J = 7.6 Hz, 1H), 6.75-6.71 (m, 1H), 5.99 (dd, J = 8.0, 1.2 Hz, 1H), 4.14 (d, J = 2.0 Hz, 2H), 3.76 (d, J = 16.4 Hz, 1H), 3.41 (d, J = 16.4 Hz, 1H), 1.16 (s, 3H), 0.54-0.47 (m, 2H),0.38- 0.32 (m, 2H). |
| 183 | MD | 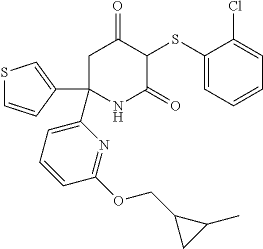 [SMILES: CC1CC1COc1cccc(C2(c3ccsc3)CC(=O)C(Sc3ccccc3Cl)C(=O)N2)n1] | 3-(2-chlorophenyl)sulfanyl- 6-[6-[(2-methylcyclo- propyl)methoxy]-2-pyridyl]- 6-(3-thienyl)piperidine- 2,4-dione was prepared in 10.4% yield according to the Example 2, Step A substituting propan-2-ol for (2-methylcyclopropyl) methanol. | ¹H NMR (400 MHz, (CD₃)₂SO) δ 11.60 (s, 1H), 8.48 (s, 1H), 7.77 (dd, J = 8.0, 8.0 Hz, 1H), 7.51 (dd, J = 2.6, 2.6 Hz, 1H), 7.33 (d, J = 2.8 Hz, 1H), 7.28 (d, J = 2.8 Hz, 1H), 7.22-7.16 (m, 2H), 6.96 (dd, J = 8.0, 8.0 Hz, 1H), 6.76 (dd, J = 8.0, 8.0 Hz, 2H), 5.84 (d, J = 8.0 Hz, 1H), 4.15-4.10 (m, 2H), 3.90 (d, J = 16.0 Hz, 1H), 3.30 (d, J = 16.0 Hz, 1H), 1.04-0.91 (m, 4H), 0.93-0.91 (m, 1H), 0.35- 0.50 (m, 1H), 0.25-0.23 (m, 1H). |
| 184 | SS | 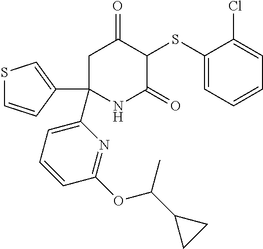 [SMILES: CC(Oc1cccc(C2(c3ccsc3)CC(=O)C(Sc3ccccc3Cl)C(=O)N2)n1)C1CC1] | 3-(2-chlorophenyl)sulfanyl- 6-[6-(1-cyclopropylethoxy)- 2-pyridyl]-6-(3-thienyl) piperidine-2,4-dione was prepared as in example 159. | ¹H NMR (400 MHz, CD₃OD) δ 7.73 (dd, J = 8.0, 4.0 Hz, 1H), 7.47 (dd, J = 5.2, 3.2 Hz, 1H), 7.30-7.20 (m, 2H), 7.17-7.12 (m, 2H), 6.96 (t, J = 4.0, 4.0 Hz, 1H), 6.81-6.71 (m, 2H), 5.97 (dd, J = 8.0, 6.8 Hz, 1H), 4.85- 4.75 (m, 1H), 3.85 (dd, J = 16.0, 2.4 Hz, 1H), 3.34-3.32 (m, 1H), 1.39-1.32 (m, 3H), 1.13- 1.10 (m, 1H), 0.49-0.29 (m, 4H). |
| 185 | SS | 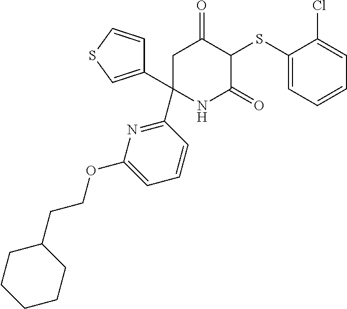 [SMILES: O=C1CC(c2ccsc2)(c2cccc(OCCC3CCCCC3)n2)NC(=O)C1Sc1ccccc1Cl] | 3-(2-chlorophenyl)sulfanyl- 6-[6-(2-cyclohexylethoxy)- 2-pyridyl]-6-(3-thienyl) piperidine-2,4-dione was prepared in 7.4% yield according to the Example 2, Step A substituting propan-2-ol for 2- cyclohexylethanol | ¹H NMR (400 MHz, CD₃OD) δ 8.48 (s, 1H), 7.74 (dd, J = 8.0, 8.0 Hz, 1H), 7.49 (dd, J = 4.0, 4.0 Hz, 1H), 7.32 (s, 1H), 7.27 (d, J = 8.0 Hz, 1H), 7.19 (d, J = 8.0 Hz, 1H), 7.13 (d, J = 4.0 Hz, 1H), 6.94 (dd, J = 8.0, 8.0 Hz, 2H), 5.80 (d, J = 8.0 Hz, 1H), 4.32-4.26 (m, 2H), 3.90 (d, J = 16.0 Hz, 1H), 3.34 (d, J = 16.0 Hz, 1H), 1.69-1.59 (m, 7H), 1.55-1.50 (m, 1H), 1.13- 1.08 (m, 3H), 0.91-0.83 (m, 2H). |
| 186 | MD | 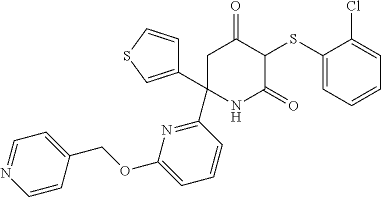 [SMILES: O=C1CC(c2ccsc2)(c2cccc(OCc3ccncc3)n2)NC(=O)C1Sc1ccccc1Cl] | 3-(2-chlorophenyl)sulfanyl- 6-[6-(4-pyridylmethoxy)- 2-pyridyl]-6-(3-thienyl) piperidine-2,4-dione was prepared in 7.3% yield according to the Example 2, Step A substituting propan-2-ol for pyridin- 4-ylmethanol | ¹H NMR (400 MHz, (CD₃)₂SO) δ 8.74 (d, J = 5.6 Hz, 1H), 8.47 (s, 1H), 7.91-7.87 (m, 3H), 7.39 (d, J = 8.0 Hz, 1H), 7.27 (d, J = 7.6 Hz, 2H), 7.00-6.72 (m, 4H), 5.76 (dd, J = 6.8, 4.0 Hz, 1H), 5.64 (d, J = 8.0 Hz, 1H), 3.87 (d, J = 16.0 Hz, 1H), 3.28 (d, J = 16.0 Hz, 1H). |
| 187 | SS | 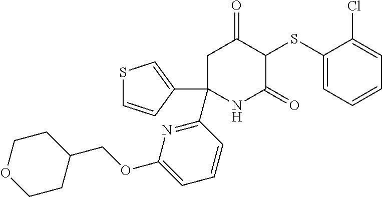 [SMILES: O=C1CC(c2ccsc2)(c2cccc(OCC3CCOCC3)n2)NC(=O)C1Sc1ccccc1Cl] | 3-(2-chlorophenyl)sulfanyl- 6-[6-(tetrahydropyran-4- ylmethoxy)-2-pyridyl]-6- (3-thienyl)piperidine-2,4- dione was prepared according to methods described therein | ¹H NMR (400 MHz, CD₃OD) δ 7.75 (dd, J = 8.0, 8.0 Hz, 1H), 7.46 (dd, J = 5.2, 3.2 Hz, 1H), 7.29-7.17 (m, 4H), 6.91 (dd, J = 7.6, 7.6 Hz, 1H), 6.81-6.74 (m, 2H), 5.95 (dd, J = 8.0, 1.6 Hz, 1H), 4.31-4.29 (m, 1H), 4.19-4.15 (m, 1H), 3.91-3.87 (m, 3H), 3.48-3.29 (m, 3H), 2.05-2.00 (m, 1H), 1.70-1.66 (m, 2H), 1.42-1.33 (m, 2H). |
| 188 | SS | 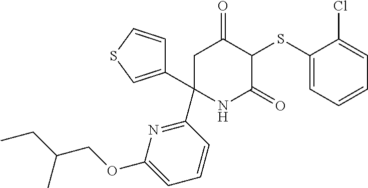 [SMILES: CCC(C)COc1cccc(C2(c3ccsc3)CC(=O)C(Sc3ccccc3Cl)C(=O)N2)n1] | 3-(2-chlorophenyl)sulfanyl- 6-[6-(2-methylbutoxy)-2- pyridyl]-6-(3-thienyl) piperidine-2,4-dione was separated from example 164. | ¹H NMR (400 MHz, (CD₃)₂SO) δ 11.6 (s, 1H), 8.48 (s, 1H), 7.74 (dd, J = 8.0, 8.0 Hz, 1H), 7.48 (dd, J = 4.0, 4.0 Hz, 1H), 7.35 (dd, J = 2.0, 2.0 Hz, 1H), 7.28 (d, J = 8.0 Hz, 1H), 7.21 (d, J = 7.6 Hz, 1H), 7.16 (d, J = 4.8 Hz, 1H), 6.92 (dd, J = 8.0, 8.0 Hz, 1H), 6.77-6.73 (m, 2H), 5.85 (d, J = 8.0 Hz, 1H), 4.17-4.07 (m, 2H), 3.90 (d, J = 16.0 Hz, 1H), 3.28 (d, J = 16.0 Hz, 1H), 1.77-1.75 (m, 1H), 1.49-1.47 (m, 1H), 1.18- 1.15 (m, 1H), 0.93-0.83 (m, 6H). |
| 189 | MD | 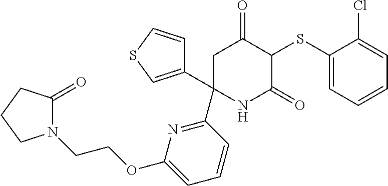 [SMILES: O=C1CC(c2ccsc2)(c2cccc(OCCN3CCCC3=O)n2)NC(=O)C1Sc1ccccc1Cl] | 3-(2-chlorophenyl)sulfanyl- 6-[6-[2-(2-oxopyrrolidin- 1-yl)ethoxy]-2-pyridyl]- 6-(3-thienyl)piperidine- 2,4-dione was prepared in 13% yield according to Example 2, Step A substituting propan-2-ol for 1-(2-hydroxyethyl) pyrrolidin-2-one. | ¹H NMR (400 MHz, (CD₃)₂SO) δ 8.52 (s, 1H), 7.79 (d, J = 8.0 Hz, 1H), 7.50 (s, 1H), 7.34 (s, 1H), 7.33-7.28 (m, 2H), 7.28 (d, J = 7.6 Hz, 1H), 7.17-7.16 (m, 1H), 6.77 (d, J = 8.4 Hz, 1H), 6.69-6.68 (m, 1H), 5.78 (d, J = 8.0 Hz, 1H), 4.39-4.36 (m, 2H), 3.94 (d, J = 16.4 Hz, 1H), 3.59- 3.56 (m, 1H), 3.45-3.41 (m, 2H), 3.32-3.27 (m, 2H), 2.16-2.12 (m, 2H), 1.82-1.76 (m, 2H). |
| 190 | MD | 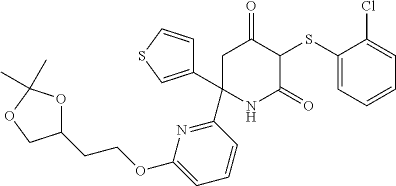 [SMILES: CC1(C)OCC(CCOc2cccc(C3(c4ccsc4)CC(=O)C(Sc4ccccc4Cl)C(=O)N3)n2)O1] | 3-(2-chlorophenyl)sulfanyl- 6-[6-[2-(2,2-dimethyl- 1,3-dioxolan-4-yl)ethoxy]- 2-pyridyl]-6-(3-thienyl) piperidine-2,4-dione was prepared in 13% yield according to the Example 2, Step A substituting propan-2-ol for 2-(2,2- dimethyl-1,3-dioxolan-4- yl)ethanol. | ¹H NMR (400 MHz, CD₃OD) δ 7.65 (dd, J = 8.0, 7.2 Hz, 1H), 7.35 (dd, J = 4.8, 2.8 Hz, 1H), 7.33- 7.28 (m, 1H), 7.16 (dd, J = 4.8, 1.2 Hz, 1H), 7.13 (d, J = 7.6 Hz, 1H), 7.09 (dd, J = 8.0, 1.2 Hz, 1H), 6.81-6.75 (m, 1H), 6.70- 6.66 (m, 2H), 6.12 (d, J = 7.6 Hz, 1H), 4.55-4.46 (m, 1H), 4.46-4.37 (m, 1H), 4.28-4.18 (m, 1H), 4.06- 3.96 (m, 1H), 3.58-3.50 (m, 1H), 3.36-3.32 (m, 2H), 2.03-1.93 (m, 2H), 1.35 (s, 3H), 1.30 (s, 3H). |
| 191 | SS | 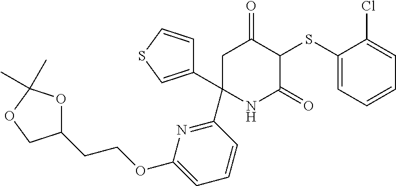 [SMILES: CC1(C)OCC(CCOc2cccc(C3(c4ccsc4)CC(=O)C(Sc4ccccc4Cl)C(=O)N3)n2)O1] | 3-(2-chlorophenyl)sulfanyl- 6-[6-[2-(2,2-dimethyl- 1,3-dioxolan-4-yl)ethoxy]- 2-pyridyl]-6-(3-thienyl) piperidine-2,4-dione was prepared in 13% yield according to the Example 2, Step A substituting propan-2-ol for 2-(2,2- dimethyl-1,3-dioxolan-4- yl)ethanol. | ¹H NMR (400 MHz, CD₃OD) δ 7.66 (dd, J = 8.0, 7.6 Hz, 1H), 7.35 (dd, J = 4.8, 2.8 Hz, 1H), 7.33-7.28 (m, 1H), 7.16 (dd, J = 4.8, 1.2 Hz, 1H), 7.13 (d, J = 7.6 Hz, 1H), 7.09 (dd, J = 8.0, 1.2 Hz, 1H), 6.80-6.76 (m, 1H), 6.70- 6.66 (m, 2H), 6.12 (d, J = 6.8 Hz, 1H), 4.55-4.46 (m, 1H), 4.46- 4.37 (m, 1H), 4.28-4.18 (m, 1H), 4.06-3.96 (m, 1H), 3.58-3.50 (m, 1H), 3.36-3.32 (m, 2H), 2.00- 1.93 (m, 2H), 1.35 (s, 3H), 1.30 (s, 3H). |
| 192 | MD | 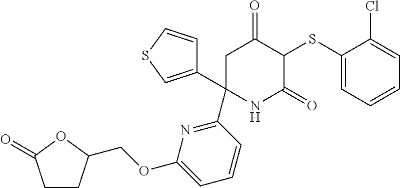 [SMILES: O=C1CCC(COc2cccc(C3(c4ccsc4)CC(=O)C(Sc4ccccc4Cl)C(=O)N3)n2)O1] | 3-(2-chlorophenyl)sulfanyl- 6-[6-[(5-oxotetrahydrofuran- 2-yl)methoxy]-2-pyridyl]- 6-(3-thienyl)piperidine- 2,4-dione was prepared in 13% yield according to Example 2, Step A substituting propan-2-ol for 5-(hydroxymethyl)dihydro- furan-2(3H)-one. | ¹H NMR (400 MHz, (CD₃)₂SO) δ 8.48 (s, 1H), 7.81 (dd, J = 8.4, 8.4 Hz, 1H), 7.50 (s, 1H), 7.33 (s, 1H), 7.27-7.23 (m, 2H), 7.16 (d, J = 5.2 Hz, 1H), 6.95 (dd, J = 7.6, 7.6 Hz, 1H), 6.83 (d, J = 8.0 Hz, 1H), 6.70 (m, 1H), 5.81 (d, J = 8.0 Hz, 1H), 4.80 (s, 1H), 4.51- 4.36 (m, 2H), 3.89 (d, J = 16.4 Hz, 1H), 3.31 (s, 1H), 2.52-2.50 (m, 2H). 2.26-2.24 (m, 1H), 1.96 (s, 1H)._ |
| 193 | SS | 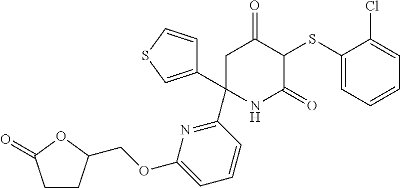 [SMILES: O=C1CCC(COc2cccc(C3(c4ccsc4)CC(=O)C(Sc4ccccc4Cl)C(=O)N3)n2)O1] | 3-(2-chlorophenyl)sulfanyl- 6-[6-[(5-oxotetrahydrofuran- 2-yl)methoxy]-2-pyridyl]- 6-(3-thienyl)piperidine- 2,4-dione was prepared in 13% yield according to Example 2, Step A substituting propan-2-ol for 5-(hydroxymethyl)dihydro- furan-2(3H)-one. | ¹H NMR (400 MHz, (CD₃)₂SO) δ 7.76-7.74 (m, 1H), 7.45-7.44 (m, 1H), 7.34 (s, 1H), 7.33 (s, 1H), 7.24-7.22 (m, 1H), 7.18- 7.16 (m, 1H), 7.15-7.14 (m, 1H), 6.85-6.84 (m, 1H), 6.77 (d, J = 8.0 Hz, 1H), 6.66-6.65 (m, 1H), 5.87 (d, J = 7.6 Hz, 1H), 4.80 (m, 1H), 4.50 (d, J = 12.0 Hz, 1H), 4.38 (d, J =12.0 Hz, 1H), 3.31 (s, 2H), 2.51-2.47 (m, 2H), 2.29-2.24 (m, 1H), 1.97-1.95 (m, 1H). |
| 194 | MD | 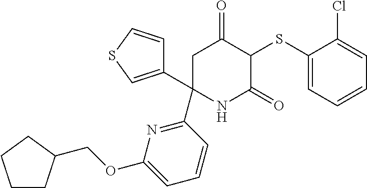 [SMILES: O=C1CC(c2ccsc2)(c2cccc(OCC3CCCC3)n2)NC(=O)C1Sc1ccccc1Cl] | 3-(2-chlorophenyl)sulfanyl- 6-[6-(cyclopentyl- methoxy)-2-pyridyl]-6-(3- thienyl)piperidine-2,4- dione was prepared in 11.1% yield according to the Example 2, Step A substituting propan-2-ol for cyclopentylmethanol. | ¹H NMR (400 MHz, (CD₃)₂SO) δ 11.54 (s, 1H), 8.37 (s, 1H), 7.70 (dd, J = 8.0, 8.0 Hz, 1H), 7.46 (dd, J = 2.6, 2.6 Hz, 1H), 7.31 (d, J = 2.8 Hz, 1H), 7.23 (dd, J = 4.0, 4.0 Hz, 1H), 7.18-7.16 (m, 2H), 7.11 (dd, J = 8.0, 8.0 Hz, 1H), 6.70 (dd, J = 4.0, 4.0 Hz, 2H), 5.80 (d, J = 8.0 Hz, 1H), 4.11 (d, J = 16.0 Hz, 2H), 3.83 (d, J = 16.0 Hz, 1H), 3.30 (d, J = 16.0 Hz, 1H), 2.22-2.20 (m, 1H), 1.68-1.65 (m, 2H), 1.51-1.43 (m, 4H), 1.36-1.24 (m, 2H). |
| 195 | MD | 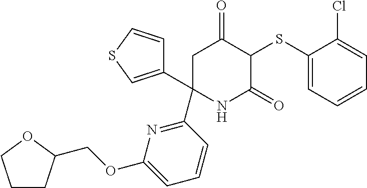 [SMILES: O=C1CC(c2ccsc2)(c2cccc(OCC3CCCO3)n2)NC(=O)C1Sc1ccccc1Cl] | 3-(2-chlorophenyl)sulfanyl- 6-[6-(tetrahydrofuran-2- ylmethoxy)-2-pyridyl]-6- (3-thienyl)piperidine-2,4- dione was prepared in 6.9% yield according to the Example 2, Step A substituting propan-2-ol for (tetrahydrofuran-2-yl) methanol | ¹H NMR (400 MHz, (CD₃)₂SO) δ 8.51 (s, 1H), 7.76 (dd, J = 8.0, 8.0 Hz, 1H), 7.50 (dd, J = 4.0, 4.0 Hz, 1H), 7.33-7.14 (m, 4H), 6.94 (dd, J = 8.0, 8.0 Hz, 1H), 6.77-6.70 (m, 2H), 5.83 (d, J = 7.6 Hz, 1H), 4.30-4.17 (m, 2H), 4.10-4.07 (m, 1H), 3.90 (d, J = 16.4 Hz, 1H), 3.76-3.69 (m, 1H), 3.61 (dd, J = 16.0, 8.0 Hz, 1H), 3.34 (d, J = 16.4 Hz, 1H), 1.98-1.89 (m, 1H), 1.84-1.72 (m, 2H), 1.63-1.55 (m, 1H). |
| 196 | SS | 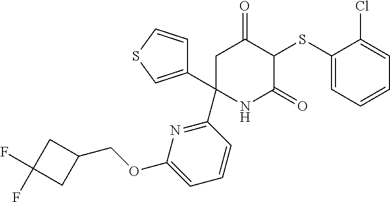 [SMILES: O=C1CC(c2ccsc2)(c2cccc(OCC3CC(F)(F)C3)n2)NC(=O)C1Sc1ccccc1Cl] | 3-(2-chlorophenyl)sulfanyl- 6-[6-[(3,3-difluorocyclo- butyl)methoxy]-2-pyridyl]- 6-(3-thienyl)piperidine- 2,4-dione was prepared in 7.3% yield according to the Example 2, Step A substituting propan-2-ol for (3,3-difluoro- cyclobutyl)methanol | ¹H NMR (400 MHz, (CD₃)₂SO) δ 7.75 (dd, J = 8.0, 8.0 Hz, 1H), 7.48 (dd, J = 4.0, 4.0 Hz, 1H), 7.33 (s, 1H), 7.23 (dd, J = 8.0, 8.0 Hz, 2H), 7.15 (d, J = 4.0 Hz, 1H), 6.91 (dd, J = 8.0, 8.0 Hz, 1H), 6.75 (d, J = 8.0 Hz, 1H), 6.68 (dd, J = 8.0, 8.0 Hz, 1H), 5.82 (d, J = 8.0 Hz, 1H), 4.33 (d, J = 4.0 Hz, 2H), 3.80 (d, J = 16.0 Hz, 1H), 3.37 (d, J = 16.0 Hz, 1H), 3.25 (d, J = 16.0 Hz, 1H) 2.66-2.58 (m, 2H), 2.40-2.30 (m, 2H). |
| 197 | SS | 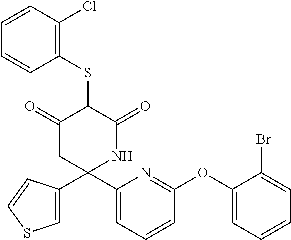 [SMILES: O=C1CC(c2ccsc2)(c2cccc(Oc3ccccc3Br)n2)NC(=O)C1Sc1ccccc1Cl] | 6-[6-(2-bromophenoxy)-2- pyridyl]-3-(2-chlorophenyl) sulfanyl-6-(3-thienyl) piperidine-2,4-dione was prepared in 6% yield according to the Example 3, Step A substituting 2-chloro-4-fluoro-phenol for 2-bromophenol. | ¹H NMR (400 MHz, CD₃OD) δ 7.88 (dd, J = 7.6, 7.6 Hz, 1H), 7.67 (d, J = 6.8 Hz, 1H), 7.39-7.30 (m, 3H), 7.15-7.13 (m, 4H), 7.03 (d, J = 2.8 Hz, 1H), 6.96 (d, J = 2.8 Hz, 1H), 6.79 (d, J = 7.6 Hz, 1H), 5.99 (d, J = 8.0 Hz, 1H), 3.51 (d, J = 16 Hz, 1H), 3.21 (d, J = 16 Hz, 1H). |
| 198 | SS | 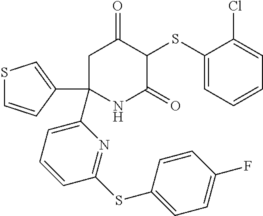 [SMILES: O=C1CC(c2ccsc2)(c2cccc(Sc3ccc(F)cc3)n2)NC(=O)C1Sc1ccccc1Cl] | 3-(2-chlorophenyl)sulfanyl- 6-[6-(4-fluorophenyl) sulfanyl-2-pyridyl]-6-(3- thienyl)piperidine-2,4- dione was prepared in 14% yield according to the Example 2, Step A substituting propan-2-ol for 4-fluorobenzenethiol. | ¹H NMR (400 MHz, CD₃OD) δ 7.64 (dd, J = 8.0, 8.0 Hz, 1H), 7.61-7.58 (m, 2H), 7.39 (dd, J = 5.2, 3.0 Hz, 1H), 7.30 (d, J = 8.0 Hz, 1H), 7.23-7.17 (m, 4H), 7.04 (dd, J = 7.6, 1H), 6.98 (dd, J = 5.1, 1.3 Hz, 1H), 6.96- 6.92 (m, 1H), 6.79-6.75 (m, 1H), 5.99 (dd, J = 8.0, 1.4 Hz, 1H), 3.68 (d, J = 16.4 Hz, 1H), 3.34 (d, J = 16.4 Hz, 1H). |
| 199 | MD | 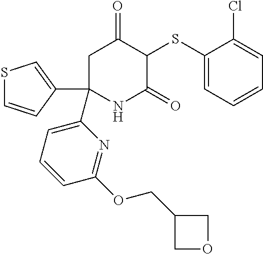 [SMILES: O=C1CC(c2ccsc2)(c2cccc(OCC3COC3)n2)NC(=O)C1Sc1ccccc1Cl] | 3-(2-chlorophenyl)sulfanyl- 6-[6-(oxetan-3-ylmethoxy)- 2-pyridyl]-6-(3-thienyl) piperidine-2,4-dione was prepared in 7.3% yield according to the Example 2, Step A substituting propan-2-ol for oxetan- 3-ylmethanol | ¹H NMR (400 MHz, CD₃OD) δ 8.55 (s, 1H), 7.77 (dd, J = 8.0, 8.0 Hz, 1H), 7.50 (dd, J = 8.0, 2.0 Hz, 1H), 7.34-7.10 (m, 3H), 6.97-6.92 (m, 1H), 6.77 (d, J = 8.0 Hz, 1H), 6.68 (dd, J = 8.0, 8.0 Hz, 1H), 5.78 (d, J = 8.0 Hz, 1H), 4.63- 4.58 (m, 2H), 4.49 (d, J = 6.8 Hz, 2H), 4.38-4.32 (m, 2H), 3.93 (d, J = 16.0 Hz, 1H), 3.35 (d, J = 16.4 Hz, 1H), 3.27 (d, J = 16.4 Hz, 1H). |
| 200 | MD | 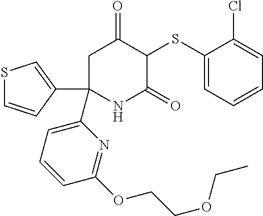 [SMILES: CCOCCOc1cccc(C2(c3ccsc3)CC(=O)C(Sc3ccccc3Cl)C(=O)N2)n1] | 3-(2-chlorophenyl)sulfanyl- 6-[6-(2-ethoxyethoxy)-2- pyridyl]-6-(3-thienyl) piperidine-2,4-dione was prepared in 41% yield according to the Example 2, Step A substituting propan-2-ol for 2- ethoxyethanol. | ¹H NMR (400 MHz, CD₃OD) δ 7.77 (dd, J = 7.6, 7.6 Hz, 1H), 7.47 (dd, J = 8.0, 4.0 Hz, 1H), 7.31- 7.28 (m, 1H), 7.24 (d, J = 8.0 Hz, 1H), 7.20 (d, J = 8.0 Hz, 1H), 7.19 (dd, J = 7.6, 0.8 Hz, 1H), 6.96 (dd, J = 7.6, 7.6 Hz, 1H), 6.84 (d, J = 8.0 Hz, 1H), 6.77 (dd, J =7.6, 7.6 Hz, 1H), 5.97 (d, J = 8.0 Hz, 1H), 4.57-4.45 (m, 2H), 3.93 (d, J = 16.4 Hz, 1H), 3.76 (t, J = 4.8 Hz, 2H), 3.55-3.49 (m, 2H), 3.48 (d, J = 16.4 Hz, 1H), 1.17 (t, J = 7.2 Hz, 3H). |
| 201 | MD | 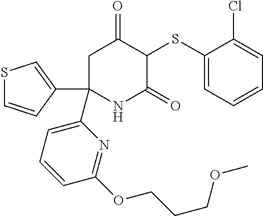 [SMILES: COCCCOc1cccc(C2(c3ccsc3)CC(=O)C(Sc3ccccc3Cl)C(=O)N2)n1] | 3-(2-chlorophenyl)sulfanyl- 6-[6-(3-methoxypropoxy)- 2-pyridyl]-6-(3-thienyl) piperidine-2,4-dione was prepared in 40% yield according to the Example 2, Step A substituting propan-2-ol for 3-methoxy- propan-1-ol. | ¹H NMR (400 MHz, CD₃OD) δ 7.71 (dd, J = 7.6, 7.6 Hz, 1H), 7.43 (dd, J = 5.2, 3.2 Hz, 1H), 7.27 (dd, J = 2.8, 1.6 Hz, 1H), 7.21 (dd, J = 7.6, 1.6 Hz, 1H), 7.15 (d, J = 5.2, 1.2 Hz, 1H), 7.14 (d, J = 7.2 Hz, 1H), 6.95-6.91 (m, 1H), 6.76 (d, J = 8.4 Hz, 1H), 6.76-6.72 (m, 1H), 5.95 (dd, J = 8.0, 1.6 Hz, 1H), 4.45-4.39 (m, 2H), 3.89 (d, J = 16.0 Hz, 1H), 3.52-3.47 (m, 2H), 3.45 (d, J = 16.0 Hz, 1H), 3.29 (s, 3H), 2.00-1.94 (m, 2H). |
| 202 | SS | 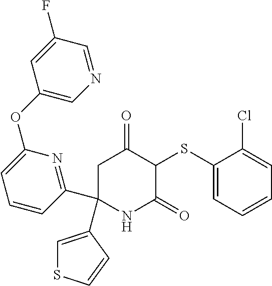 [SMILES: O=C1CC(c2ccsc2)(c2cccc(Oc3cncc(F)c3)n2)NC(=O)C1Sc1ccccc1Cl] | 3-(2-chlorophenyl)sulfanyl- 6-[6-[(5-fluoro-3-pyridyl) oxy]-2-pyridyl]-6-(3-thienyl) piperidine-2,4-dione was prepared in 6.9% yield according to the Example 3, Step A substituting 2-Chloro-4-fluoro-phenol for 5-fluoropyridin-3-ol | ¹H NMR (400 MHz, (CD₃)₂SO) δ 8.46 (s, 1H), 8.33 (s, 1H), 8.00 (dd, J = 8.0, 8.0 Hz, 1H), 7.56 (d, J = 8.0 Hz, 1H), 7.47 (dd, J = 8.0, 8.0 Hz, 2H), 7.28 (d, J = 8.0 Hz, 1H), 7.23 (d, J = 4.0 Hz, 1H), 7.13 (d, J = 8.0 Hz, 1H), 7.01-6.94 (m, 2H), 6.78 (dd, J = 7.6, 7.6 Hz, 1H), 5.88 (d, J = 7.6 Hz, 1H), 3.47 (d, J = 16.0 Hz, 1H), 3.23 (d, J = 16.0 Hz, 1H). |
| 203 | SS | 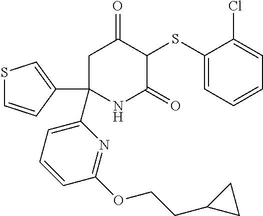 [SMILES: O=C1CC(c2ccsc2)(c2cccc(OCCC3CC3)n2)NC(=O)C1Sc1ccccc1Cl] | 3-(2-chlorophenoxy)-6- [6-(2-cyclopropylethoxy)-2- pyridyl]-6-(3-thienyl) piperidine-2,4-dione was prepared according to methods described therein | ¹H NMR (400 MHz, CD₃OD) δ 7.65 (dd, J = 8.0. 8.0 Hz, 1H), 7.36 (dd, J = 8.0, 4.0 Hz, 1H), 7.26-7.23 (m, 2H), 7.10-7.08 (m, 2H), 6.79-6.76 (m, 2H), 6.69 (d, J = 8.0 Hz, 1H), 5.94 (dd, J = 8.0, 4.0 Hz, 1H), 4.41-4.34 (m, 2H), 3.74 (d, J = 16.0 Hz, 1H), 3.35 (d, J = 16.0 Hz, 1H), 1.61-1.56 (m, 2H), 0.78-0.74 (m, 1H), 0.38-0.35 (m, 2H), 0.04-0.01 (m, 2H). |
| 204 | SS | 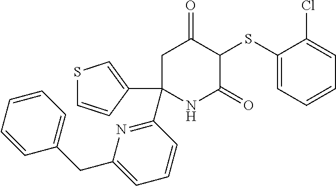 [SMILES: O=C1CC(c2ccsc2)(c2cccc(Cc3ccccc3)n2)NC(=O)C1Sc1ccccc1Cl] | 6-(6-benzyl-2-pyridyl)-3- (2-chlorophenyl)sulfanyl- 6-(3-thienyl)piperidine-2,4- dione was prepared according to methods described therein | ¹H NMR (400 MHz, (CD₃)₂SO) δ 11.65 (s, 1H), 8.13 (s, 1H), 7.72 (dd, J = 8.0, 8.0 Hz, 1H), 7.44 (d, J = 2.0 Hz, 2H ), 7.28-7.08 (m, 9H), 6.87 (dd, J = 8.0, 8.0 Hz, 1H), 6.57 (dd, J = 8.0, 8.0 Hz, 1H), 5.86 (d, J = 8.0 Hz, 1H), 4.07 (s, 2H), 3.79 (d, J = 16.0 Hz, 1H), 3.31 (d, J = 16.0 Hz, 1H). |
| 205 | SS | 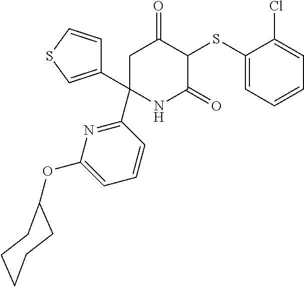 [SMILES: O=C1CC(c2ccsc2)(c2cccc(OC3CCCCC3)n2)NC(=O)C1Sc1ccccc1Cl] | 3-(2-chlorophenyl)sulfanyl- 6-[6-(cyclohexoxy)-2- pyridyl]-6-(3-thienyl) piperidine-2,4-dione was separated from example 157. | ¹H NMR (400 MHz, (CD₃)₂SO) δ 7.70 (dd, J = 8.0, 8.0 Hz, 1H), 7.48 (dd, J = 2.0, 2.0 Hz, 1H), 7.32 (dd, J = 1.4, 1.4 Hz, 1H), 7.24 (dd, J = 4.0, 4.0 Hz, 1H), 7.18- 7.13 (m, 2H), 6.92 (dd, J = 4.0, 4.0 Hz, 1H), 6.70-6.65 (m, 2H), 5.86 (d, J = 8.0 Hz, 1H), 5.03-4.99 (m, 1H), 3.73 (d, J = 16.0 H, 1H), 3.13 (d, J = 16.0 Hz 1H), 1.85-1.49 (m, 4H), 1.47-1.21 (m, 6H). |
| 206 | SS | 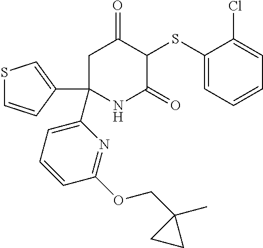 [SMILES: CC1(COc2cccc(C3(c4ccsc4)CC(=O)C(Sc4ccccc4Cl)C(=O)N3)n2)CC1] | 3-(2-chlorophenyl)sulfanyl- 6-[6-[(1-methylcyclopropyl) methoxy]-2-pyridyl]-6-(3- thienyl)piperidine-2,4-dione was prepared in 7% yield according to the Example 2, Step A substituting propan-2-ol for (1-methyl- cyclopropyl)methanol. | ¹H NMR (400 MHz, CD₃OD) δ 7.69 (dd, J = 8.0, 7.2 Hz, 1H), 7.41 (dd, J = 5.2, 3.2 Hz, 1H), 7.27 (dd, J = 5.2, 1.2 Hz, 1H), 7.18 (dd, J = 8.0, 1.2 Hz, 1H), 7.13 (dd, J = 2.8, 1.2 Hz, 1H), 7.12 (d, J = 7.2 Hz, 1H), 6.92- 6.87 (m, 1H), 6.76 (d, J = 7.6 Hz, 1H), 6.75-6.71 (m, 1H), 6.00 (d, J = 8.0, 1.2 Hz, 1H), 4.14 (d, J = 1.6 Hz, 2H), 3.75 (d, J = 16.0 Hz, 1H), 3.41 (d, J = 16.4 Hz, 1H), 1.16 (s, 3H), 0.54-0.47 (m, 2H), 0.37-0.31 (m, 2H). |
| 207 | SS | 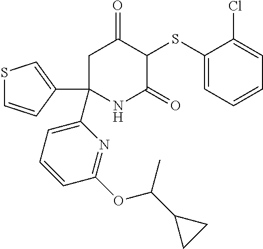 [SMILES: CC(Oc1cccc(C2(c3ccsc3)CC(=O)C(Sc3ccccc3Cl)C(=O)N2)n1)C1CC1] | 3-(2-chlorophenyl)sulfanyl- 6-[6-(1-cyclopropylethoxy)- 2-pyridyl]-6-(3-thienyl) piperidine-2,4-dione was prepared as in example 159. | ¹H NMR (400 MHz, CD₃OD) δ 7.71 (dd, J = 8.0, 4.0 Hz, 1H), 7.49 (dd, J = 5.2, 3.2 Hz, 1H), 7.30-7.20 (m, 2H), 7.17-7.12 (m, 2H), 6.96 (dd, J = 8.0, 4.0 Hz, 1H), 6.81-6.71 (m, 2H), 5.97 (dd, J = 8.0, 6.8 Hz, 1H), 4.81- 4.77 (m, 1H), 3.84 (d, J = 16.0 Hz, 1H), 3.44 (d, J = 16.0 Hz, 1H), 1.38 (d, J = 5.1 Hz, 1H), 1.13-1.10 (m, 1H), 0.49-0.22 (m, 4H). |
| 208 | SS | 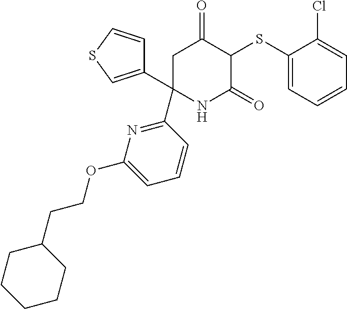 [SMILES: O=C1CC(c2ccsc2)(c2cccc(OCCC3CCCCC3)n2)NC(=O)C1Sc1ccccc1Cl] | 3-(2-chlorophenyl)sulfanyl- 6-[6-(2-cyclohexylethoxy)- 2-pyridyl]-6-(3-thienyl) piperidine-2,4-dione was prepared in 7.4% yield according to the Example 2, Step A substituting propan-2-ol for 2-cyclo- hexylethanol | ¹H NMR (400 MHz, CD₃OD) δ 8.48 (s, 1H), 7.74 (dd, J = 8.0, 8.0 Hz, 1H), 7.49 (dd, J = 4.0, 4.0 Hz, 1H), 7.32 (s, 1H), 7.27 (d, J = 8.0 Hz, 1H), 7.19 (d, J = 8.0 Hz, 1H), 7.13 (d, J = 4.0 Hz, 1H), 6.94 (dd, J = 8.0, 8.0 Hz, 2H), 5.80 (d, J = 8.0 Hz, 1H), 4.32-4.26 (m, 2H), 3.90 (d, J = 16.0 Hz, 1H), 3.34 (d, J = 16.0 Hz, 1H), 1.69-1.59 (m, 7H), 1.55-1.50 (m, 1H), 1.13-1.08 (m, 3H), 0.91-0.83 (m, 2H). |
| 209 | SS | 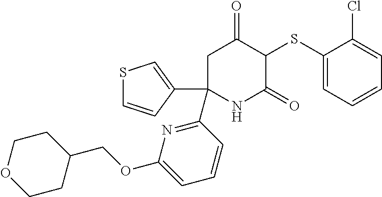 [SMILES: O=C1CC(c2ccsc2)(c2cccc(OCC3CCOCC3)n2)NC(=O)C1Sc1ccccc1Cl] | 3-(2-chlorophenyl)sulfanyl- 6-[6-(tetrahydropyran-4- ylmethoxy)-2-pyridyl]-6- (3-thienyl)piperidine-2,4- dione was prepared according to methods described therein. | ¹H NMR (400 MHz, CD₃OD) δ 7.76 (dd, J = 8.0, 8.0 Hz, 1H), 7.46 (dd, J = 5.2, 3.2 Hz, 1H), 7.30-7.17 (m, 4H), 6.95 (dd, J = 7.6, 7.6 Hz, 1H), 6.81-6.79 (m, 2H), 5.96 (dd, J = 8.0, 1.6 Hz, 1H), 4.33-4.32 (m, 1H), 4.19-4.17 (m, 1H), 3.92-3.88 (m, 3H), 3.48-3.32 (m, 3H), 2.03-2.02 (m, 1H), 1.70-1.67 (m, 2H), 1.41-1.31 (m, 2H). |
| 210 | SS | 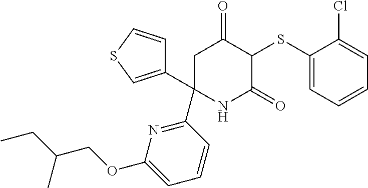 [SMILES: CCC(C)COc1cccc(C2(c3ccsc3)CC(=O)C(Sc3ccccc3Cl)C(=O)N2)n1] | 3-(2-chlorophenyl)sulfanyl- 6-[6-(2-methylbutoxy)-2- pyridyl]-6-(3-thienyl) piperidine-2,4-dione was separated from example 164. | ¹H NMR (400 MHz, (CD₃)₂SO) δ 11.6 (s, 1H), 8.46 (s, 1H), 7.76 (dd, J = 8.0, 8.0 Hz, 1H), 7.51 (dd, J = 4.0, 4.0 Hz, 1H), 7.35 (d, J = 1.6 Hz, 1H), 7.27 (d, J = 8.0 Hz, 1H), 7.21 (d, J = 8.4 Hz, 1H), 7.16 (d, J = 5.2 Hz, 1H), 6.92 (dd, J = 8.0, 8.0 Hz, 1H), 6.77-6.72 (m, 2H), 5.84 (d, J = 8.0 Hz, 1H), 4.19-4.05 (m, 2H), 3.89 (d, J = 16.0 Hz, 1H), 3.35 (d, J = 16.0 Hz, 1H), 1.78-1.75 (m, 1H), 1.47- 1.45 (m, 1H), 1.19-1.16 (m, 1H), 0.91-0.83 (m, 6H). |
| 211 | SS | 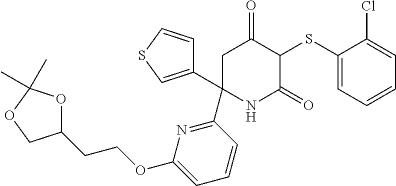 [SMILES: CC1(C)OCC(CCOc2cccc(C3(c4ccsc4)CC(=O)C(Sc4ccccc4Cl)C(=O)N3)n2)O1] | 3-(2-chlorophenyl)sulfanyl- 6-[6-[2-(2,2-dimethyl-1,3- dioxolan-4-yl)ethoxy]-2- pyridyl]-6-(3-thienyl) piperidine-2,4-dione was prepared in 13% yield according to the Example 2, Step A substituting propan-2-ol for 2-(2,2- dimethyl-1,3-dioxolan-4- yl)ethanol. | ¹H NMR (400 MHz, CD₃OD) δ 7.58 (dd, J = 8.0, 7.6 Hz, 1H), 7.28 (dd, J = 4.8, 3.2 Hz, 1H), 7.22-7.22 (m, 1H), 7.07 (dd, J = 5.2, 1.2 Hz, 1H), 7.05 (d, J = 8.0 Hz, 1H), 7.02 (dd, J = 8.0, 1.2 Hz, 1H), 6.73- 6.69 (m, 1H), 6.62-6.58 (m, 2H), 6.03-5.99 (m, 1H), 4.46-4.39 (m, 1H), 4.36-4.30 (m, 1H), 4.18-4.11 (m, 1H), 3.94-3.89 (m, 1H), 3.49- 3.36 (m, 2H), 3.26-3.22 (m, 1H), 1.91-1.86 (m, 2H), 1.26 (s, 3H), 1.21 (s, 3H). |
| 212 | SS | 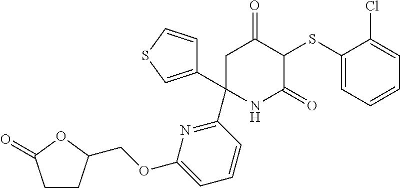 [SMILES: O=C1CCC(COc2cccc(C3(c4ccsc4)CC(=O)C(Sc4ccccc4Cl)C(=O)N3)n2)O1] | 3-(2-chlorophenyl)sulfanyl- 6-[6-[(5-oxotetrahydrofuran- 2-yl)methoxy]-2-pyridyl]-6- (3-thienyl)piperidine-2,4- dione was prepared in 13% yield according to Example 2, Step A substituting propan-2-ol for 5-(hydroxy- methyl)dihydrofuran- 2(3H)-one. | ¹H NMR (400 MHz, (CD₃)₂SO) δ 7.76 (dd, J = 7.6, 7.6 Hz, 1H), 7.45 (s, 1H), 7.35 (s, 1H), 7.24-7.16 (m, 3H), 6.87 (m, 1H), 6.78 (d, J = 8.4 Hz, 1H), 6.67 (m, 1H), 5.87 (m, 1H), 4.80 (s, 1H), 4.52 (d, J = 12.0 Hz, 1H), 4.36 (d, J = 12.0 Hz, 1H), 3.31 (s, 1H), 2.47-2.46 (m, 2H), 2.26-2.21 (m, 1H), 1.96-1.94 (m, 1H). |
| 213 | SS | 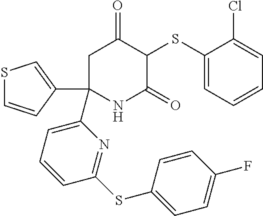 [SMILES: O=C1CC(c2ccsc2)(c2cccc(Sc3ccc(F)cc3)n2)NC(=O)C1Sc1ccccc1Cl] | 3-(2-chlorophenyl)sulfanyl- 6-[6-(4-fluorophenyl) sulfanyl-2-pyridyl]-6-(3- thienyl)piperidine-2,4- dione was prepared in 14% yield according to the Example 2, Step A substituting propan-2-ol for 4-fluorobenzenethiol. | ¹H NMR (400 MHz, CD₃OD) δ 7.64 (dd, J = 8.0, 8.0 Hz, 1H), 7.61-7.58 (m, 2H), 7.39 (dd, J = 5.2, 3.0 Hz, 1H), 7.30 (d, J = 8.0 Hz, 1H), 7.23-7.17 (m, 4H), 7.04 (d, J = 8.0, 1H), 6.98 (dd, J = 5.1, 1.3 Hz, 1H), 6.96- 6.92 (m, 1H), 6.79-6.75 (m, 1H), 5.99 (dd, J = 8.0, 1.4 Hz, 1H), 3.68 (d, J = 16.4 Hz, 1H), 3.34 (d, J = 16.4 Hz, 1H). |
| 214 | SS | 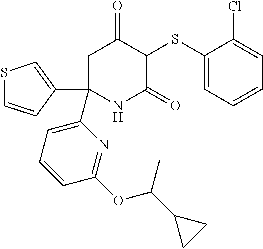 [SMILES: CC(Oc1cccc(C2(c3ccsc3)CC(=O)C(Sc3ccccc3Cl)C(=O)N2)n1)C1CC1] | 3-(2-chlorophenyl)sulfanyl- 6-[6-(1-cyclopropyl- ethoxy)-2-pyridyl]-6-(3- thienyl)piperidine-2,4-dione was prepared as in example 159. | ¹H NMR (400 MHz, CD₃OD) δ 7.72 (dd, J = 8.0, 4.0 Hz, 1H), 7.45 (dd, J = 5.2, 3.2 Hz, 1H), 7.30-7.20 (m, 2H), 7.17-7.12 (m, 2H), 6.96 (t, J = 4.0, 4.0 Hz, 1H), 6.81-6.71 (m, 2H), 5.97 (dd, J = 8.0, 6.8 Hz, 1H), 4.83-4.79 (m, 1H), 3.81 (d, J = 16.0 Hz, 1H), 3.46 (d, J = 16.0 Hz, 1H), 1.33 (d, J = 6.4 Hz, 1H), 1.13- 1.10 (m, 1H), 0.55-0.34 (m, 4H). |
| 215 | SS | 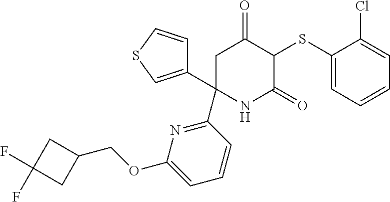 [SMILES: O=C1CC(c2ccsc2)(c2cccc(OCC3CC(F)(F)C3)n2)NC(=O)C1Sc1ccccc1Cl] | 3-(2-chlorophenyl)sulfanyl- 6-[6-[(3,3-difluorocyclo- butyl)methoxy]-2-pyridyl]- 6-(3-thienyl)piperidine- 2,4-dione was prepared in 7.3% yield according to the Example 2, Step A substituting propan-2-ol for (3,3-difluorocyclo- butyl)methanol | ¹H NMR (400 MHz, (CD₃)₂SO) δ 8.23 (s, 1H), 7.75 (dd, J = 8.0, 8.0 Hz, 1H), 7.48 (dd, J = 4.0, 4.0 Hz, 1H), 7.33 (d, J = 4.0 Hz, 1H), 7.25-7.21 (m, 2H), 7.15 (d, J = 4.0 Hz, 1H), 6.91 (dd, J = 8.0, 8.0 Hz, 1H), 6.74 (d, J = 8.0 Hz, 1H), 6.68 (dd, J = 8.0, 8.0 Hz, 1H), 5.82 (d, J = 8.0 Hz, 1H), 4.32 (d, J = 8.0 Hz, 2H), 3.78 (d, J = 16.0 Hz, 1H), 3.31 (d, J = 16.0 Hz, 1H), 3.24 (d, J = 16.0 Hz, 1H) 2.67-2.57 (m, 2H), 2.40-2.31 (m, 2H). |
| 216 | SS | 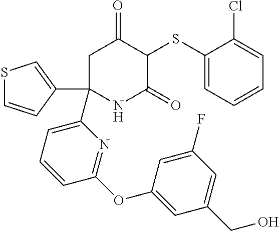 [SMILES: O=C1CC(c2ccsc2)(c2cccc(Oc3cc(F)cc(CO)c3)n2)NC(=O)C1Sc1ccccc1Cl] | 3-(2-chlorophenyl)sulfanyl- 6-[6-[3-fluoro-5-(hydroxy- methyl)phenoxy]-2-pyridyl]- 6-(3-thienyl)piperidine-2,4- dione was prepared in 7.2% yield according to the Example 3, Step A substituting 2-Chloro-4- fluoro-phenol for 3-fluoro- 5-(hydroxymethyl)phenol | ¹H NMR (400 MHz, CD₃OD) δ 7.87 (dd, J = 8.0, 8.0 Hz, 1H), 7.36 (d, J = 8.0 Hz,2H), 7.17-7.09 (m, 2H), 7.04 (d, J = 4.0 Hz, 1H), 6.98-6.86 (m, 4H), 6.76-6.72 (m, 2H), 6.07 (d, J = 8.0 Hz, 1H), 4.55 (d, J = 7.6 Hz, 2H), 3.48 (d, J = 16.0 Hz, 1H), 3.27 (d, J = 16.0 Hz, 1H). |
| 217 | MD | 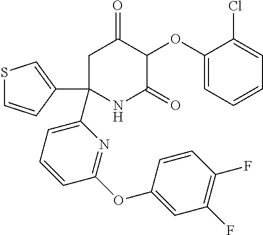 [SMILES: O=C1CC(c2ccsc2)(c2cccc(Oc3ccc(F)c(F)c3)n2)NC(=O)C1Oc1ccccc1Cl] | 3-(2-chlorophenoxy)-6-[6- (3,4-difluorophenoxy)-2- pyridyl]-6-(3-thienyl) piperidine-2,4-dione was prepared in 16.6% yield according to the Example 3, Step A substituting 2-chloro-4-fluoro-phenol for 3,4-difluorophenol. | ¹H NMR (400 MHz, (CD₃)₂SO) δ 7.92 (dd, J = 7.6, 7.6 Hz, 1H), 7.46-7.43 (m, 3H), 7.34-7.27 (m, 3H), 7.03-6.98 (m, 3H), 6.91 (dd, J = 8.0, 8.0 Hz, 1H), 6.84 (dd, J = 8.0, 8.0 Hz, 1H), 6.09 (d, J = 1.6 Hz, 1H), 3.36, (d, J = 16.0 Hz, 1H), 3.09 (d, J = 16.0 Hz, 1H). |
| 218 | MD | 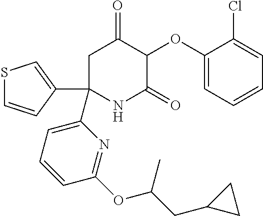 [SMILES: CC(CC1CC1)Oc1cccc(C2(c3ccsc3)CC(=O)C(Oc3ccccc3Cl)C(=O)N2)n1] | 3-((2-chlorophenyl)thio)- 6-(6-((1-cyclopropylpropan- 2-yl)oxy)pyridin-2-yl)-6- (thiophen-3-yl)piperidine- 2,4-dione was prepared in 33% yield according to the Example 2, Step a substituting propan-2-ol for 1-cyclopropylpropan-2-ol | ¹H NMR (400 MHz, CD₃OD) δ 7.69 (dd, J = 8.0, 4.0 Hz, 1H), 7.42 (dd, J = 4.4, 2.8 Hz, 1H), 7.14- 7.10 (m, 2H), 6.93 (d, J = 2.0 Hz, 1H), 7.20 (d, J = 2.8, 1H), 7.27- 7.25 (m, 2H), 7.14-7.10 (m, 2H), 6.91 (dd, J = 4.0, 2.0 Hz, 1H), 6.75-6.73 (m, 2H), 5.98 (dd, J = 9.2, 1.6 Hz, 1H), 5.41-5.34 (m, 1H), 3.89 (d, J = 16.4 Hz, 1H), 3.45 (d, J = 16.4 Hz, 1H), 1.65- 1.60 (m, 1H), 1.43-1.28 (m, 4H), 0.73-0.69 (m, 1H), 0.40-0.38 (m, 2H), 0.07-0.00 (m, 2H). |
| 219 | MD | 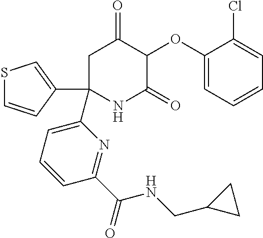 [SMILES: O=C(NCC1CC1)c1cccc(C2(c3ccsc3)CC(=O)C(Oc3ccccc3Cl)C(=O)N2)n1] | 6-(5-((2-chlorophenyl)thio)- 4,6-dioxo-2-(thiophen-3-yl) piperidin-2-yl)-N-(cyclo- propylmethyl)picolinamide was prepared in 12% yield according to the Example 6, Step A substituting (4-fluorophenyl)boronic acid for cyclopropyl- methanamine. | ¹H NMR (400 MHz, CD₃OD) δ 8.85 (s, 1H), 8.15 (d, J = 8.0, 8.0 Hz, 1H), 8.08 (dd, J = 8.0, 8.0 Hz, 1H), 7.79 (d, J = 8.0 Hz, 1H), 7.51 (dd, J = 5.2, 2.0 Hz, 1H), 7.38 (dd, J = 1.2, 1.6 Hz, 1H), 7.23 (d, J = 8.0 Hz, 1H), 7.18 (dd, J = 5.2, 1.2 Hz, 1H), 6.95 (dd, J = 8.0, 1.6 Hz, 1H), 6.71 (dd, J = 8.0, 8.0 Hz, 1H), 5.91 (dd, J = 8.0, 1.6 Hz, 1H), 3.87 (d, J = 16.4 Hz, 1H), 3.62 (d, J = 16.4 Hz, 1H), 3.32-3.28 (m, 2H), 1.09-1.05 (m, 1H), 0.51-0.48 (m, 2H), 0.29-0.26 (m, 2H). |
| 220 | MD | 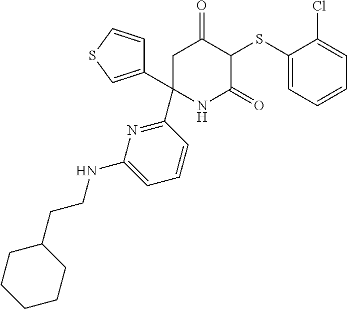 [SMILES: O=C1CC(c2ccsc2)(c2cccc(NCCC3CCCCC3)n2)NC(=O)C1Sc1ccccc1Cl] | 3-(2-chlorophenyl)sulfanyl- 6-[6-(2-cyclohexyl- ethylamino)-2-pyridyl]- 6-(3-thienyl)piperidine-2,4- dione was prepared in 3% yield according to the Example 4, Step A substituting cyclo- hexanamine for 2- cyclohexylethanamine. | ¹H NMR (400 MHz, CD₃OD) δ 7.71 (dd, J = 7.6, 7.6 Hz, 1H), 7.45 (d, J = 2.0 Hz, 1H), 7.23 (d, J = 2.8 Hz, 1H), 7.21-7.18 (m, 3H), 6.96 (dd, J = 8.0, 8.0 Hz, 1H), 6.80 (dd, J = 8.0, 8.0 Hz, 1H), 6.58 (d, J = 7.2 Hz, 1H), 6.05 (d, J = 8.0 Hz, 1H), 3.78 (d, J = 16.0 Hz, 1H), 3.49 (d, J = 16.0 Hz, 1H), 3.38 (t, J = 7.5 Hz, 2H), 1.92- 1.72 (m, 5H), 1.50-1.45 (m, 2H), 1.37-1.23 (m, 4H), 1.20-1.96 (m, 2H). |
| 221 | SS | 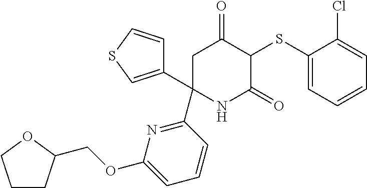 [SMILES: O=C1CC(c2ccsc2)(c2cccc(OCC3CCCO3)n2)NC(=O)C1Sc1ccccc1Cl] | 3-(2-chlorophenyl)sulfanyl- 6-[6-(tetrahydrofuran-2- ylmethoxy)-2-pyridyl]-6- (3-thienyl)piperidine-2,4- dione was prepared in 6.9% yield according to the Example 2, Step A substituting propan-2-ol for (tetrahydrofuran-2-yl) methanol | ¹H NMR (400 MHz, CD₃OD) δ 7.70 (dd, J = 8.0, 8.0 Hz, 1H), 7.39 (dd, J = 4.0, 4.0 Hz, 1H), 7.28 (s, 1H), 7.15 (dd, J = 4.0, 4.0 Hz, 3H), 6.86 (dd, J = 8.0, 8.0 Hz, 1H), 6.73 (dd, J = 16.0, 8.0 Hz, 2H), 6.05 (d, J = 8.0 Hz, 1H), 4.41 (dd, J = 12.0, 4.0 Hz, 1H), 4.31-4.20 (m, 2H), 3.86 (dd, J = 16.0, 8.0 Hz, 1H), 3.75 (dd, J = 12.0, 8.0 Hz, 1H), 3.66 (d, J = 16.0 Hz, 1H),3.38 (d, J = 16.0 Hz, 1H), 2.06-2.00 (m, 1H), 1.97-1.84 (m, 2H), 1.76-1.67 (m, 1H). |
| 222 | MD | 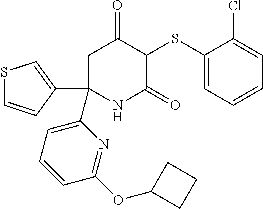 [SMILES: O=C1CC(c2ccsc2)(c2cccc(OC3CCC3)n2)NC(=O)C1Sc1ccccc1Cl] | 3-(2-chlorophenyl)sulfanyl- 6-[6-(cyclobutoxy)-2- pyridyl]-6-(3-thienyl) piperidine-2,4-dione was prepared in 10.2% yield according to the Example 2, Step A substituting propan-2-ol for cyclo- butanol. | ¹H NMR (400 MHz, (CD₃)₂SO) δ 8.45 (s, 1H), 7.73 (dd, J = 8.0, 8.0 Hz, 1H), 7.48 (d, J = 2.8 Hz, 1H), 7.33 (dd, J = 4.4, 1.6 Hz, 1H), 7.22 (d, J = 8.0 Hz, 1H), 7.20 (d, J = 8.0 Hz, 1H), 7.13 (dd, J = 5.2, 1.6 Hz, 1H), 6.94 (dd, J = 8.0, 8.0 Hz, 1H), 6.72-6.68 (m, 2H), 5.81 (d, J = 8.0 Hz, 1H), 5.16- 5.13 (m, 1H), 3.87 (d, J = 16.0 Hz, 1H), 3.34 (d, J = 16.0 Hz, 1H), 2.40-2.38 (m, 2H), 1.99- 1.96 (m, 2H), 1.73-1.60 (m, 2H). |
| 223 | MD | 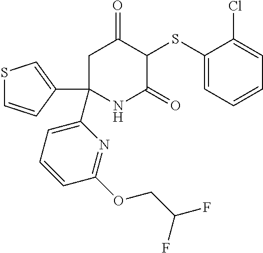 [SMILES: O=C1CC(c2ccsc2)(c2cccc(OCC(F)F)n2)NC(=O)C1Sc1ccccc1Cl] | 3-(2-chlorophenyl)sulfanyl- 6-[6-(2,2-difluoroethoxy)- 2-pyridyl]-6-(3-thienyl) piperidine-2,4-dione was prepared in 20% yield according to Example 2, Step A substituting propan-2-ol for 2,2- difluoroethanol. | ¹H NMR (400 MHz, CD₃OD) δ 7.81 (dd, J = 8.0, 8.0 Hz, 1H), 7.45-7.44 (m, 1H), 7.27-7.23 (m, 3H), 7.16-7.14 (m, 1H), 6.88 (dd, J = 8.4 Hz, 1H), 6.74-6.73 (m, 1H), 6.12-6.11 (m, 1H), 5.96 (d, J = 8.4 Hz, 1H), 4.86-4.50 (m, 2H), 3.87 (d, J = 16.4 Hz, 1H), 3.49 (d, J = 16.4 Hz, 1H). |
| 224 | MD | 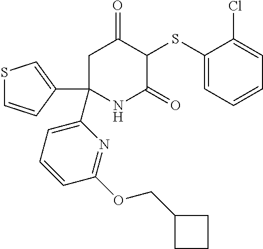 [SMILES: O=C1CC(c2ccsc2)(c2cccc(OCC3CCC3)n2)NC(=O)C1Sc1ccccc1Cl] | 3-(2-chlorophenyl)sulfanyl- 6-[6-(cyclobutylmethoxy)- 2-pyridyl]-6-(3-thienyl) piperidine-2,4-dione was prepared in 11.7% yield according to the Example 2, Step A substituting propan-2-ol for cyclo- butylmethanol. | ¹H NMR (400 MHz, (CD₃)₂SO) δ 8.45 (s, 1H), 7.75 (dd, J = 8.0, 8.0 Hz, 1H), 7.49 (d, J = 2.8 Hz, 1H), 7.33 (dd, J = 4.4, 1.6 Hz, 1H), 7.21 (d, J = 8.0 Hz, 1H), 7.19 (d, J = 8.0 Hz, 1H), 7.15 (dd, J = 5.2, 1.6 Hz, 1H), 6.92 (dd, J = 8.0, 8.0 Hz, 1H), 6.75-6.69 (m, 2H), 5.83 (d, J = 8.0 Hz, 1H), 4.24 (d, J = 8.0 Hz, 1H), 3.91 (d, J = 16.4 Hz, 2H), 3.33 (d, J = 16.4 Hz, 1H), 2.65-2.59 (m, 1H), 2.00- 1.95 (m, 2H), 1.84-1.75 (m, 4H). |
| 225 | SS | 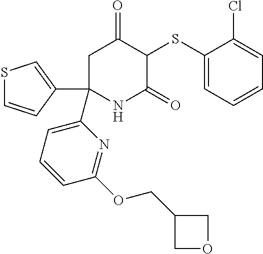 [SMILES: O=C1CC(c2ccsc2)(c2cccc(OCC3COC3)n2)NC(=O)C1Sc1ccccc1Cl] | 3-(2-chlorophenyl)sulfanyl- 6-[6-(oxetan-3-ylmethoxy)- 2-pyridyl]-6-(3-thienyl) piperidine-2,4-dione was prepared in 7.3% yield according to the Example 2, Step A substituting propan-2-ol for oxetan-3- ylmethanol | ¹H NMR (400 MHz, CD₃OD) δ 7.69 (dd, J = 8.0, 8.0 Hz, 1H), 7.38 (dd, J = 4.0, 4.0 Hz, 1H), 7.29 (s, 1H), 7.17-7.12 (m, 3H), 6.82 (dd, J = 8.0, 8.0 Hz, 1H), 6.74-6.66 (m, 2H), 6.08 (d, J = 7.6 Hz, 1H), 4.80-4.77 (m, 2H), 4.59-4.53 (m, 4H), 3.55-3.29 (m, 3H). |
| 226 | MD | 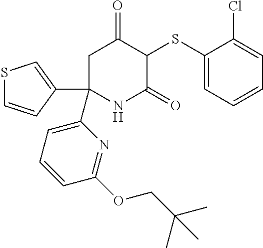 [SMILES: CC(C)(C)COc1cccc(C2(c3ccsc3)CC(=O)C(Sc3ccccc3Cl)C(=O)N2)n1] | 3-(2-chlorophenyl)sulfanyl- 6-[6-(2,2-dimethylpropoxy)- 2-pyridyl]-6-(3-thienyl) piperidine-2,4-dione was prepared in 7.0% yield according to the Example 2, Step A substituting propan-2-ol for 2,2- dimethylpropan-1-ol. | ¹H NMR (400 MHz, (CD₃)₂SO) δ 8.47 (s, 1H), 7.75 (dd, J = 8.0, 8.0 Hz, 1H), 7.50 (dd, J = 2.6. 2.6 Hz, 1H), 7.32 (d, J = 2.0 Hz, 1H), 7.26 (d, J = 8.0 Hz, 1H), 7.19 (d, J = 8.0 Hz, 1H), 7.16 (d, J = 11.2 Hz, 1H), 6.90 (dd, J = 8.0, 8.0 Hz, 1H), 6.77-6.71 (m, 2H), 5.84 (d, J = 8.0 Hz, 1H), 4.00 (d, J = 10.4 Hz, 2H), 3.90 (d, J = 16.0 Hz, 1H), 3.45 (d, J = 16.0 Hz, 1H), 0.93 (s, 9H). |
| 227 | SS | 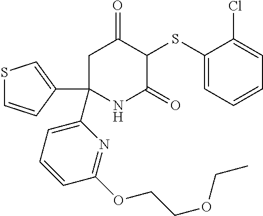 [SMILES: CCOCCOc1cccc(C2(c3ccsc3)CC(=O)C(Sc3ccccc3Cl)C(=O)N2)n1] | 3-(2-chlorophenyl)sulfanyl- 6-[6-(2-ethoxyethoxy)-2- pyridyl]-6-(3-thienyl) piperidine-2,4-dione was prepared in 38% yield according to the Example 2, Step A substituting propan-2-ol for 2- ethoxyethanol. | ¹H NMR (400 MHz, CD₃OD) δ 7.70 (dd, J = 8.0, 8.0 Hz, 1H), 7.40 (dd, J = 5.2, 2.8 Hz, 1H), 7.28- 7.26 (m, 1H), 7.18-7.15 (m, 3H), 6.87 (dd, J = 7.2, 7.2 Hz, 1H), 6.76 (d, J = 8.0 Hz, 1H), 6.71 (dd, J = 7.6, 7.6 Hz, 1H), 6.01 (d, J = 8.0 Hz, 1H), 4.51-4.48 (m, 2H), 3.75-3.70 (m, 3H), 3.52-3.47 (m, 2H), 3.40 (d, J = 16.0 Hz, 1H), 1.14 (t, J = 7.2 Hz, 3H). |
| 228 | SS | 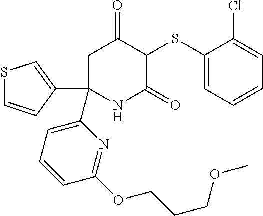 [SMILES: COCCCOc1cccc(C2(c3ccsc3)CC(=O)C(Sc3ccccc3Cl)C(=O)N2)n1] | 3-(2-chlorophenyl)sulfanyl- 6-[6-(3-methoxypropoxy)-2- pyridyl]-6-(3-thienyl) piperidine-2,4-dione was prepared in 40% yield according to the Example 2, Step A substituting propan-2-ol for 3- methoxypropan-1-ol | ¹H NMR (400 MHz, CD₃OD) δ 7.68 (dd, J = 8.4, 8.4 Hz, 1H), 7.40 (dd, J = 4.8, 3.2 Hz, 1H), 7.28 (dd, J = 4.8, 3.2 Hz, 1H), 7.18-7.12 (m, 3H), 6.89-6.85 (m, 1H), 6.73- 6.69 (m, 2H), 6.01 (dd, J = 8.0, 1.2 Hz, 1H), 4.45-4.39 (m, 2H), 3.72 (d, J = 16.0 Hz, 1H), 3.52- 3.48 (m, 2H), 3.40 (d, J = 16.0 Hz, 1H), 3.29 (s, 3H), 2.00-1.94 (m, 2H). |
| 229 | MD | 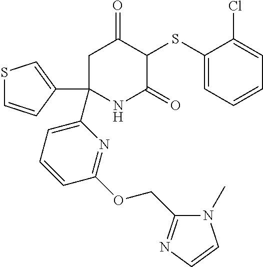 [SMILES: Cn1ccnc1COc1cccc(C2(c3ccsc3)CC(=O)C(Sc3ccccc3Cl)C(=O)N2)n1] | 3-(2-chlorophenyl)sulfanyl- 6-[6-[(1-methylimidazol- 2-yl)methoxy]-2-pyridyl]- 6-(3-thienyl)piperidine- 2,4-dione was prepared in 10% yield according to Example 2, Step A substituting propan-2-ol for (1-methyl-1H-imidazol- 2-yl)methanol. | ¹H NMR (400 MHz, (CD₃)₂SO) δ 8.48 (s, 1H), 7.82 (dd, J = 8.0, 8.0 Hz, 1H), 4.77-7.46 (m, 1H), 7.35 (s, 1H), 7.25-7.23 (m, 2H), 7.19 (s, 1H), 7.09-7.07 (m, 1H), 6.91- 6.85 (m, 2H), 6.83 (d, J = 8.4 Hz, 1H), 6.64-6.63 (m, 1H), 5.74 (d, J = 7.6 Hz, 1H), 5.46 (d, J = 13.2 Hz, 1H), 5.34 (d, J = 12.8 Hz, 1H), 3.85 (d, J = 15.6 Hz, 1H), 3.26 (d, J = 15.6 Hz, 1H). |
| 230 | MD | 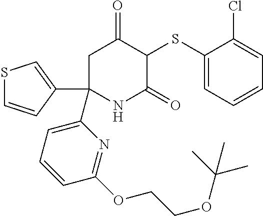 [SMILES: CC(C)(C)OCCOc1cccc(C2(c3ccsc3)CC(=O)C(Sc3ccccc3Cl)C(=O)N2)n1] | 6-[6-(2-tert-butoxyethoxy)- 2-pyridyl]-3-(2-chloro- phenyl)sulfanyl-6-(3- thienyl)piperidine-2,4- dione was prepared in 9.4% yield according to the Example 2, Step A substituting propan-2-ol for 2-(tert-butoxy)ethanol | ¹H NMR (400 MHz, (CD₃)₂SO) δ 8,50 (s, 1H), 7.77 (dd, J = 8.0, 8.0 Hz, 1H), 7.49 (d, J = 2.0 Hz, 1H), 7.32 (d, J = 2.8 Hz, 1H), 7.26 (d, J = 2.8 Hz, 1H), 7.26-7.21 (m, 2H), 7.15 (dd, J = 8.0, 8.0 Hz, 1H), 6.74 (dd, J = 8.0, 8.0 Hz, 2H), 5.85 (dd, J = 8.0, 2.4 Hz, 1H), 4.33 (t, J = 4.4 Hz, 2H), 3.91 (d, J = 16.0 Hz, 1H), 3.57 (t, J = 5.2 Hz, 2H), 3.32 (d, J = 16.0 Hz, 1H), 1.07 (s, 9H). |
| 231 | MD | 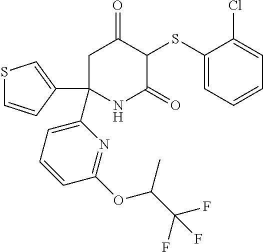 [SMILES: CC(Oc1cccc(C2(c3ccsc3)CC(=O)C(Sc3ccccc3Cl)C(=O)N2)n1)C(F)(F)F] | 3-(2-chlorophenyl)sulfanyl- 6-(3-thienyl)-6-[6-(2,2,2- trifluoro-1-methyl-ethoxy)- 2-pyridyl]piperidine-2,4- dione was prepared in 20% yield according to Example 2, Step A substituting propan-2-ol for 1,1,1-trifluoropropan- 2-ol. | ¹H NMR (400 MHz, CD₃OD) δ 7.83-7.81 (m, 1H), 7.45-7.44 (m, 1H), 7.28-7.26 (m, 3H), 7.22-7.20 (m, 1H), 6.93-6.85 (m, 1H), 6.71-6.70 (m, 1H), 5.93-5.82 (m, 2H), 3.88-3.29 (m, 2H), 1.44-1.36 (m, 3H). |
| 232 | SS | 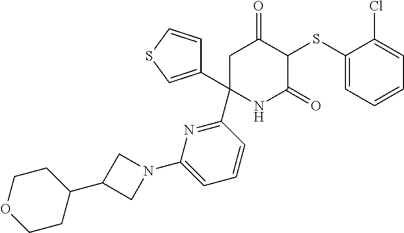 [SMILES: O=C1CC(c2ccsc2)(c2cccc(N3CC(C4CCOCC4)C3)n2)NC(=O)C1Sc1ccccc1Cl] | 3-(2-chlorophenyl)sulfanyl- 6-[6-(3-tetrahydropyran- 4-ylazetidin-1-yl)-2- pyridyl]-6-(3-thienyl) piperidine-2,4-dione was prepared in 10% yield according to the Example 4, Step A substituting cyclo- hexanamine for 3- (tetrahydro-2H-pyran-4- yl)azetidine. | ¹H NMR (400 MHz, CD₃OD) δ 7.53 (dd, J = 7.6, 7.6 Hz, 1H), 7.38 (d, J = 2.8 Hz, 1H), 7.27 (d, J = 2.8 Hz, 1H), 7.19 (d, J = 8.0 Hz, 1H), 7.11 (d, J = 7.6 Hz, 1H), 6.78 (d, J = 7.4 Hz, 1H), 6.78- 6.73 (m, 2H), 6.33 (d, J = 7.2 Hz, 1H), 5.96 (d, J = 8.0 Hz, 1H), 4.09-4.05 (m, 2H), 3.91-3.87 (m, 2H), 3.75-3.71 (m, 3H), 3.39- 3.34 (m, 3H), 2.46-2.44 (m, 1H), 1.60-1.53 (m, 3H), 1.21-1.16 (m, 2H). |
| 233 | MD | 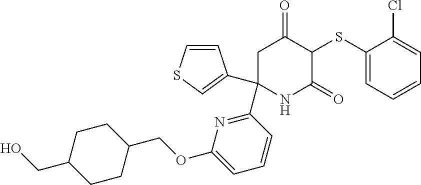 [SMILES: O=C1CC(c2ccsc2)(c2cccc(OCC3CCC(CO)CC3)n2)NC(=O)C1Sc1ccccc1Cl] | 3-((2-chlorophenyl)thio)- 6-(6-((4-(hydroxymethyl) cyclohexyl)methoxy) pyridin-2-yl)-6-(thiophen- 3-yl)piperidine-2,4-dione was prepared in 35% yield according to the Example 2, Step A substituting propan-2-ol for cyclo- hexane-1,4-diyldimethanol | ¹H NMR (400 MHz, CD₃OD) δ 7.73 (dd, J = 8.0, 8.0 Hz, 1H), 7.46 (dd, J = 5.2, 3.2 Hz, 1H), 7.29-7.16 (m, 4H), 6.94 (dd, J = 8.0, 8.0 Hz, 1H), 6.77-6.74 (m, 2H), 5.97 (dd, J = 8.0, 1.6 Hz, 1H), 4.29-4.25 (m, 1H), 4.17-4.15 (m, 1H), 3.92 (d, J = 16.8 Hz, 1H), 3.47 (d, J = 16.4 Hz, 1H), 1.90-1.79 (m, 5H), 1.43-1.41 (m, 1H), 1.10-0.89 (m, 4H). |
| 234 | SS | 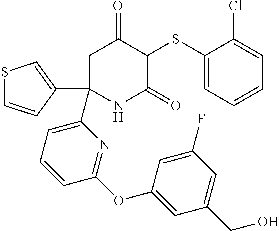 [SMILES: O=C1CC(c2ccsc2)(c2cccc(Oc3cc(F)cc(CO)c3)n2)NC(=O)C1Sc1ccccc1Cl] | 3-(2-chlorophenyl)sulfanyl- 6-[6-[3-fluoro-5-(hydroxy- methyl)phenoxy]-2-pyridyl]- 6-(3-thienyl)piperidine- 2,4-dione was prepared in 7.2% yield according to the Example 3, Step A substituting 2-Chloro-4- fluoro-phenol for 3-fluoro- 5-(hydroxymethyl)phenol | ¹H NMR (400 MHz, CD₃OD) δ 7.85 (dd, J = 8.0, 8.0 Hz, 1H), 7.37-7.33 (m, 2H), 7.21 (s, 1H), 7.13 (d, J = 8.0 Hz, 1H), 7.04 (d, J = 8.0 Hz, 1H), 6.96-6.90 (m, 3H), 6.83 (dd, J = 8.0, 8.0 Hz, 1H), 6.72 (dd, J = 8.0, 8.0 Hz, 1H), 6.10 (d, J = 8.0 Hz, 1H), 4.55 (dd, J = 16.0, 12.0 Hz, 1H), 3.41 (d, J = 16.0 Hz, 1H), 3.25 (d, J = 12.0 Hz, 1H). |
| 235 | SS | 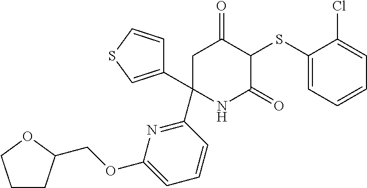 [SMILES: O=C1CC(c2ccsc2)(c2cccc(OCC3CCCO3)n2)NC(=O)C1Sc1ccccc1Cl] | 3-(2-chlorophenyl)sulfanyl- 6-[6-(tetrahydrofuran- 2-ylmethoxy)-2-pyridyl]- 6-(3-thienyl)piperidine- 2,4-dione was prepared in 6.9% yield according to the Example 2, Step A substituting propan-2-ol for (tetrahydrofuran-2- yl)methanol | ¹H NMR (400 MHz, CD₃OD) δ 7.67 (dd, J = 8.0, 8.0 Hz, 1H), 7.37 (dd, J = 4.0, 4.0 Hz, 1H), 7.29 (s, 1H), 7.14 (dd, J = 4.0, 4.0 Hz, 3H), 6.82 (dd, J = 8.0, 8.0 Hz, 1H), 6.72-6.66 (m, 2H), 6.10 (d, J = 8.0 Hz, 1H), 4.42 (dd, J = 12.0, 4.0 Hz, 1H), 4.30-4.21 (m, 2H), 3.85 (dd, J = 16.0, 8.0 Hz, 1H), 3.75 (dd, J = 12.0, 8.0 Hz, 1H), 3.53 (d, J = 16.0 Hz, 1H), 3.35 (d, J = 16.0 Hz, 1H), 2.05-1.99 (m, 1H), 1.95-1.86 (m, 2H), 1.77-1.70 (m, 1H). |
| 236 | SS | 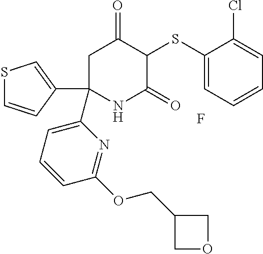 | 3-(2-chlorophenyl)sulfanyl- 6-[6-(oxetan-3-ylmethoxy)- 2-pyridyl]-6-(3-thienyl) piperidine-2,4-dione was prepared in 7.3% yield according to the Example 2, Step A substituting propan-2-ol for oxetan- 3-ylmethanol | ¹H NMR (400 MHz, CD₃OD) δ 7.69 (dd, J = 8.0, 8.0 Hz, 1H), 7.38 (dd, J = 4.0, 4.0 Hz, 1H), 7.29 (s, 1H), 7.17-7.12 (m, 3H), 6.82 (dd, J = 8.0, 8.0 Hz, 1H), 6.74-6.66 (m, 2H), 6.08 (d, J = 7.6 Hz, 1H), 4.80-4.77 (m, 2H), 4.59-4.53 (m, 4H), 3.55-3.29 (m, 3H). |
| 237 | SS | 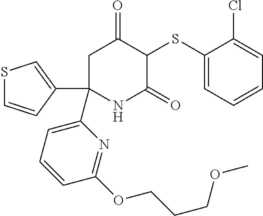 [SMILES: COCCCOc1cccc(C2(c3ccsc3)CC(=O)C(Sc3ccccc3Cl)C(=O)N2)n1] | 3-(2-chlorophenyl)sulfanyl- 6-[6-(3-methoxypropoxy)- 2-pyridyl]-6-(3-thienyl) piperidine-2,4-dione was prepared in 40% yield according to the Example 2, Step A substituting propan-2-ol for 3- methoxypropan-1-ol | ¹H NMR (400 MHz, CD₃OD) δ 7.69 (dd, J = 8.0, 8.0 Hz, 1H), 7.41 (dd, J = 4.8, 2.8 Hz, 1H), 7.27 (dd, J = 3.2, 1.2 Hz, 1H), 7.20-7.13 (m, 3H), 6.92-6.87 (m, 1H), 6.75-6.70 (m, 2H), 5.98 (dd, J = 8.0, 1.2 Hz, 1H), 4.44- 4.40 (m, 2H), 3.79 (d, J = 16.4 Hz, 1H), 3.52-3.49 (m, 2H), 3.43 (d, J = 16.4 Hz, 1H), 3.29 (s, 3H), 2.00-1.94 (m, 2H). |
| 238 | SS | 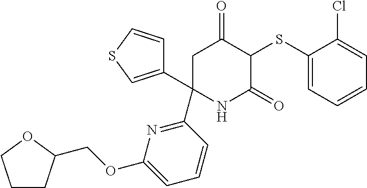 [SMILES: O=C1CC(c2ccsc2)(c2cccc(OCC3CCCO3)n2)NC(=O)C1Sc1ccccc1Cl] | 3-(2-chlorophenyl)sulfanyl- 6-[6-(tetrahydrofuran-2- ylmethoxy)-2-pyridyl]-6- (3-thienyl)piperidine-2,4- dione was prepared in 6.9% yield according to the Example 2, Step A substituting propan-2-ol for (tetrahydrofuran-2- yl)methanol | ¹H NMR (400 MHz, CD₃OD) δ 7.68 (dd, J = 8.0, 8.0 Hz, 1H), 7.38 (dd, J = 4.0, 4.0 Hz, 1H), 7.29 (s, 1H), 7.14 (dd, J = 4.0, 4.0 Hz, 3H), 6.83 (dd, J = 8.0, 8.0 Hz, 1H), 6.72 (dd, J = 16.0, J = 8.0 Hz, 2H), 6.09 (d, J = 8.0 Hz, 1H), 4.41 (dd, J = 12.0, 4.0 Hz, 1H), 4.31-4.21 (m, 2H), 3.85 (dd, J = 16.0, 8.0 Hz, 1H), 3.75 (dd, J = 12.0, 8.0 Hz, 1H), 3.56 (d, J = 16.0 Hz, 1H), 3.36 (d, J = 16.0 Hz, 1H), 2.06-1.99 (m, 1H), 1.95-1.86 (m, 2H), 1.77-1.70 (m, 1H). |
| 239 | SS | 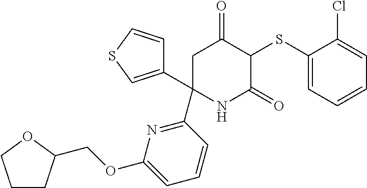 [SMILES: O=C1CC(c2ccsc2)(c2cccc(OCC3CCCO3)n2)NC(=O)C1Sc1ccccc1Cl] | 3-(2-chlorophenyl)sulfanyl- 6-[6-(tetrahydrofuran-2- ylmethoxy)-2-pyridyl]-6- (3-thienyl)piperidine-2,4- dione was prepared in 6.9% yield according to the Example 2, Step A substituting propan-2-ol for (tetrahydrofuran-2- yl)methanol | ¹H NMR (400 MHz, CD₃OD) δ 7.68 (dd, J = 8.0, 8.0 Hz, 1H), 7.38 (dd, J = 4.0, 4.0 Hz, 1H), 7.29 (s, 1H), 7.14 (dd, J = 4.0, 4.0 Hz, 3H), 6.83 (dd, J = 8.0, 8.0 Hz, 1H), 6.72 (dd, J = 16.0, 8.0 Hz, 2H), 6.09 (d, J = 8.0 Hz, 1H), 4.41 (dd, J = 12.0, 4.0 Hz, 1H), 4.31-4.20 (m, 2H), 3.86 (dd, J = 16.0, 8.0 Hz, 1H), 3.75 (dd, J = 12.0, 8.0 Hz, 1H), 3.56 (d, J = 16.0 Hz, 1H), 3.35 (d, J = 16.0 Hz, 1H), 2.09-2.01 (m, 1H), 1.97- 1.85 (m, 2H), 1.76-1.69 (m, 1H). |
| 240 | SS | 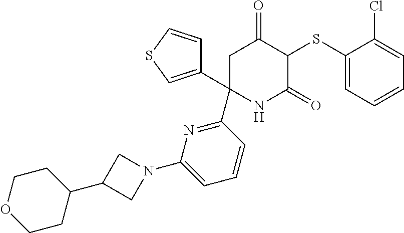 [SMILES: O=C1CC(c2ccsc2)(c2cccc(N3CC(C4CCOCC4)C3)n2)NC(=O)C1Sc1ccccc1Cl] | 3-(2-chlorophenyl)sulfanyl- 6-[6-(3-tetrahydropyran- 4-ylazetidin-1-yl)-2- pyridyl]-6-(3-thienyl) piperidine-2,4-dione was prepared in 10% yield according to the Example 4, Step A substituting cyclohexanamine for 3- (tetrahydro-2H-pyran-4- yl)azetidine. | ¹H NMR (400 MHz, CD₃OD) δ 7.54 (dd, J = 7.6, 7.6 Hz, 1H), 7.40 (d, J = 2.8 Hz, 1H), 7.27 (d, J = 2.8 Hz, 1H), 7.21 (d, J = 8.0 Hz, 1H), 7.11 (d, J = 7.6 Hz, 1H), 6.92 (d, J = 7.4 Hz, 1H), 6.80-6.74 (m, 2H), 6.34 (d, J = 7.2 Hz, 1H), 5.93 (d, J = 8.0 Hz, 1H), 4.07- 3.91 (m, 2H), 3.88-3.67 (m, 5H), 3.40-3.34 (m, 3H), 2.46-2.44 (m, 1H), 1.59-1.52 (m, 3H), 1.21- 1.16 (m, 2H). |
| 241 | SS | 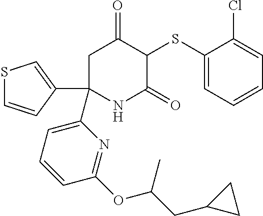 [SMILES: CC(CC1CC1)Oc1cccc(C2(c3ccsc3)CC(=O)C(Sc3ccccc3Cl)C(=O)N2)n1] | 3-(2-chlorophenyl)sulfanyl- 6-[6-(2-cyclopropyl-1- methyl-ethoxy)-2-pyridyl]- 6-(3-thienyl)piperidine- 2,4-dione was prepared according to methods described therein | ¹H NMR (400 MHz, CD₃OD) δ 7.73 (dd, J = 8.4, 4.0 Hz, 1H), 7.46 (d, J = 3.2 Hz, 1H), 7.31 (d, J = 1.6 Hz, 1H), 7.19-7.14 (m, 3H), 6.94 (dd, J = 8.0, 4.0 Hz, 1H), 6.74-6.72 (m, 2H), 6.06 (dd, J = 8, 1.2 Hz, 1H), 5.45-5.40 (m, 1H), 3.86 (d, J = 16.4 Hz, 1H), 3.47 (d, J = 16.4 Hz, 1H), 1.70-1.65 (m, 1H), 1.47-1.33 (m, 4H), 0.76-0.73 (m, 1H), 0.36- 0.32 (m, 2H), 0.06-0.01 (m, 2H). |
| 242 | SS | 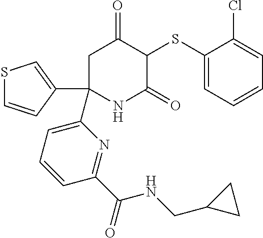 [SMILES: O=C(NCC1CC1)c1cccc(C2(c3ccsc3)CC(=O)C(Sc3ccccc3Cl)C(=O)N2)n1] | 6-[5-(2-chlorophenyl) sulfanyl-4,6-dioxo-2-(3- thienyl)-2-piperidyl]-N- (cyclopropylmethyl) pyridine-2-carboxamide was prepared according to methods described therein | ¹H NMR (400 MHz, CD₃OD) δ 7.89 (d, J = 8.0, 8.0 Hz, 1H), 7.81 (dd, J = 8.0, 8.0 Hz, 1H), 7.53 (d, J = 8.0 Hz, 1H), 7.25 (dd, J = 5.2, 2.0 Hz, 1H), 7.13 (dd, J = 7.2, 1.6 Hz, 1H), 6.97-6.91 (m, 2H), 6.68 (dd, J = 8.0, 1.6 Hz, 1H), 6.46 (dd, J = 8.0, 8.0 Hz, 1H), 5.66 (dd, J = 8.0, 1.6 Hz, 1H), 3.60 (d, J = 16.4 Hz, 1H) 3.35 (d, J = 16.4 Hz, 1H), 3.04-3.02 (m, 2H), 0.84-0.80 (m, 1H), 0.25-0.22 (m, 2H), 0.04-0.01 (m, 2H). |
| 243 | SS | 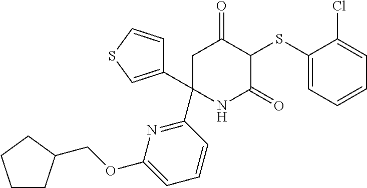 [SMILES: O=C1CC(c2ccsc2)(c2cccc(OCC3CCCC3)n2)NC(=O)C1Sc1ccccc1Cl] | 3-(2-chlorophenyl)sulfanyl- 6-[6-(cyclopentylmethoxy)- 2-pyridyl]-6-(3-thienyl) piperidine-2,4-dione was separated from example 194. | ¹H NMR (400 MHz, CD₃OD) δ 7.70 (dd, J = 8.0, 8.0 Hz, 1H), 7.41 (dd, J = 2.6, 2.6 Hz, 1H), 7.27 (d, J = 2.8 Hz, 1H), 7.19 (dd, J = 4.0, 4.0 Hz, 1H), 7.16-7.12 (m, 2H), 6.91 (dd, J = 8.0, 8.0 Hz, 1H), 6.73 (dd, J = 4.0, 4.0 Hz, 2H), 6.00 (d, J = 8.0 Hz, 1H), 4.26-4.20 (m, 2H), 3.81 (d, J = 16.0 Hz, 1H), 3.43 (d, J = 16.4 Hz, 1H), 2.35-2.29 (m, 1H), 1.79- 1.63 (m, 2H), 1.62-1.54 (m, 4H), 1.36-1.33 (m, 2H). |
| 244 | SS | 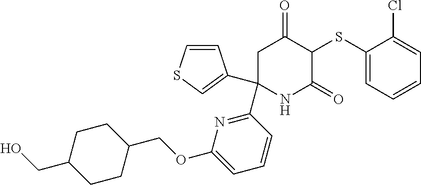 [SMILES: O=C1CC(c2ccsc2)(c2cccc(OCC3CCC(CO)CC3)n2)NC(=O)C1Sc1ccccc1Cl] | 3-(2-chlorophenyl)sulfanyl- 6-[6-[[4-(hydroxymethyl) cyclohexyl]methoxy]-2- pyridyl]-6-(3-thienyl) piperidine-2,4-dione was prepared according to methods described herein. | ¹H NMR (400 MHz, CD₃OD) δ 7.70 (dd, J = 8.0, 8.0 Hz, 1H), 7.41 (dd, J = 5.2, 3.2 Hz, 1H), 7.25-7.12 (m, 4H), 6.94 (dd, J = 8.0, 8.0 Hz, 1H), 6.75-6.70 (m, 2H), 5.95 (dd, J = 8.0, 1.6 Hz, 1H), 4.25-4.21 (m, 1H), 4.15- 4.12 (m, 1H), 3.88 (d, J = 16.4 Hz, 1H), 3.43 (d, J = 16.4 Hz, 1H), 1.86-1.68 (m, 5H), 1.38- 1.34 (m, 1H), 1.06-0.86 (m, 4H). |
| 245 | SS | 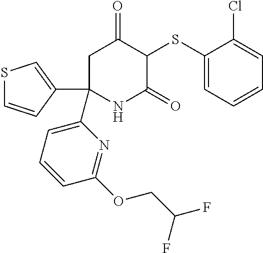 [SMILES: O=C1CC(c2ccsc2)(c2cccc(OCC(F)F)n2)NC(=O)C1Sc1ccccc1Cl] | 3-(2-chlorophenyl)sulfanyl- 6-[6-(2,2-difluoroethoxy)- 2-pyridyl]-6-(3-thienyl) piperidine-2,4-dione was prepared in 20% yield according to Example 2, Step A substituting propan- 2-ol for 2,2-difluoroethanol. | ¹H NMR (400 MHz, (CD₃)₂SO) δ 8.40 (s, 1H), 7.84 (dd, J = 7.6, 7.6 Hz, 1H), 7.48-7.35 (m, 1H), 7.35 (s, 1H), 7.30-7.25 (m, 2H), 7.24 (d, 1H), 7.17 (d, J = 3.6 Hz, 1H), 6.88 (dd, J = 8.4, 8.4 Hz, 1H), 6.71-6.70 (m, 1H), 6.33- 6.32 (m, 1H), 5.82 (d, J = 7.6 Hz, 1H), 4.61-4.50 (m, 2H), 3.85 (d, J = 16.0 Hz, 1H), 3.32 (d, J = 16.0 Hz, 1H). |
| 246 | MD | 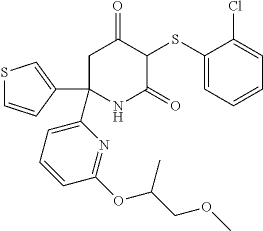 [SMILES: COCC(C)Oc1cccc(C2(c3ccsc3)CC(=O)C(Sc3ccccc3Cl)C(=O)N2)n1] | 3-(2-chlorophenyl)sulfanyl- 6-[6-(2-methoxy-1-methyl- ethoxy)-2-pyridyl]-6-(3- thienyl)piperidine-2,4- dione was prepared in 8.8% yield according to the Example 2, Step A substituting propan-2-ol for 1-methoxypropan-2-ol | ¹H NMR (400 MHz, (CD₃)₂SO) δ 7.72 (dd, J = 8.0, 8.0 Hz, 1H), 7.44 (s, 1H), 7.27-7.16 (m, 4H), 6.94 (dd, J = 8.0, 8.0 Hz, 1H), 6.75-6.74 (m, 2H), 5.97 (d, J = 8.0 Hz, 1H), 5.47-5.44 (m, 1H), 3.90 (d, J = 16.0 Hz, 1H), 3.57- 3.48 (m, 3H), 3.36 (s, 2H), 3.25 (s, 1H), 1.32-1.24 (m, 3H). |
| 247 | MD | 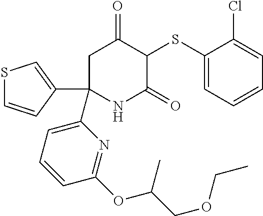 [SMILES: CCOCC(C)Oc1cccc(C2(c3ccsc3)CC(=O)C(Sc3ccccc3Cl)C(=O)N2)n1] | 3-((2-chlorophenyl)thio)- 6-(6-((1-ethoxypropan-2- yl)oxy)pyridin-2-yl)-6- (thiophen-3-yl)piperidine- 2,4-dione was prepared in 6% yield according to the Example 2, Step A substituting propan-2-ol for 1-ethoxypropan-2-ol | ¹H NMR (400 MHz, CD₃OD) δ 7.71 (dd, J = 8.4, 4.0 Hz, 1H), 7.42 (d, J = 2.4 Hz, 1H), 7.25- 7.14 (m, 4H), 6.95 (dd, J = 4.0, 4.0 Hz, 1H), 6.75-6.73 (m, 2H), 5.95 (dd, J = 8.0, 1.8 Hz 1H), 5.45-5.42 (m, 1H), 4.25-4.23 (m, 1H), 3.90 (dd, J = 16.0, 1.2 Hz 1H), 3.59-3.43 (m, 5H), 1.31-l.05 (m, 6H). |
| 248 | SS | 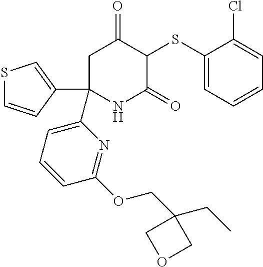 [SMILES: CCC1(COc2cccc(C3(c4ccsc4)CC(=O)C(Sc4ccccc4Cl)C(=O)N3)n2)COC1] | (6S)-3-((2-chlorophenyl) thio)-6-(6-((3-ethyloxetan- 3-yl)-6-(thiophen-3-yl) piperidine-2,4-dione was prepared in 11% yield according to the Example 2, Step A substituting propan-2-ol for (3-ethyl- oxetan-3-yl)methanol | ¹H NMR (400 MHz, CD₃OD) δ 7.73 (dd, J = 8.0, 8.0 Hz, 1H), 7.43 (dd, J = 8.0, 8.0 Hz, 1H), 7.27 (d, J = 2.8 Hz, 1H), 7.19- 7.14 (m, 2H), 6.84 (dd, J = 8.0, 8.0 Hz, 1H), 6.82 (d, J = 8.4 m, 1H), 6.72 (dd, J = 8.0, 8.0 Hz, 1H), 5.93 (dd, J = 8.0, 2.4 Hz, 1H), 4.55-4041 (m, 6H), 3.88 (d, J = 16.4 Hz, 1H), 3.46 (d, J = 16.4 Hz, 1H), 1.81 (dd, J = 14.8, 3.2 Hz, 2H), 0.88 (t, J = 7.2 Hz, 3H). |
| 249 | MD | 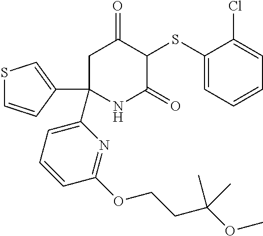 [SMILES: COC(C)(C)CCOc1cccc(C2(c3ccsc3)CC(=O)C(Sc3ccccc3Cl)C(=O)N2)n1] | 3-(2-chlorophenyl)sulfanyl- 6-[6-(3-methoxy-3-methyl- butoxy)-2-pyridyl]-6-(3- thienyl)piperidine-2,4-dione was prepared in 6.3% yield according to the Example 2, Step A substituting propan-2-ol for 2-methoxy- 2-methylpropan-1-ol | ¹H NMR (400 MHz, CD₃OD) δ 7.58 (d, J = 7.6 Hz, 1H), 7.46 (dd, J = 4.8, 1.2 Hz, 1H), 7.29-7.16 (m, 4H), 6.96 (s, 1H), 6.77 (d, J = 8.8 Hz, 1H), 5.99 (d, J = 8.0 Hz, 1H), 4.49-4.45 (m, 1H), 3.95 (d, J = 16.4 Hz, 1H), 3.46 (d, J = 16.4 Hz, 1H), 3.20 (s, 3H), 1.97 (t, J = 7.2 Hz, 2H), 1.22 (d, J = 4.0 Hz, 6H). |
| 250 | MD | 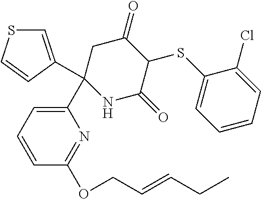 [SMILES: CC/C=C/COc1cccc(C2(c3ccsc3)CC(=O)C(Sc3ccccc3Cl)C(=O)N2)n1] | 3-(2-chlorophenyl)sulfanyl- 6-(6-pent-2-enoxy-2- pyridyl)-6-(3-thienyl) piperidine-2,4-dione was prepared in 12% yield according to the Example 2, Step A substituting propan-2-ol for (E)- pent-2-en-1-ol. | ¹H NMR (400 MHz, CD₃OD) δ 7.71 (dd, J = 7.6, 7.6 Hz, 1H), 7.42 (d, J = 2.0 Hz, 1H), 7.25 (d, J = 2.8 Hz, 1H), 7.19 (d, J = 2.8 Hz, 1H), 7.15-6.92 (m, 2H), 6.92 (dd, J = 8.0, 8.0 Hz, 1H), 6.75 (dd, J = 8.0, 8.0 Hz, 2H), 5.96 (d, J = 8.0 Hz, 1H), 5.85- 5.82 (m, 1H), 5.81-5.65 (m, 1H), 4.80 (d, J = 6.4 Hz, 2H), 3.89 (d, J = 16.0 Hz, 1H), 3.45 (d, J = 16.0 Hz, 1H), 2.05-1.98 (m, 2H), 0.95 (t, J = 7.2 Hz, 3H). |
| 251 | SS | 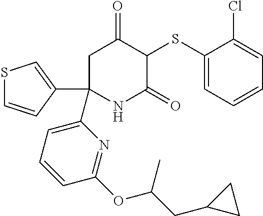 [SMILES: CC(CC1CC1)Oc1cccc(C2(c3ccsc3)CC(=O)C(Sc3ccccc3Cl)C(=O)N2)n1] | 3-(2-chlorophenyl)sulfanyl- 6-[6-(2-cyclopropyl-1- methyl-ethoxy)-2- pyridyl]-6-(3-thienyl) piperidine-2,4-dione was prepared according to methods described therein. | ¹H NMR (400 MHz, CD₃OD) δ 7.67 (dd, J = 8.0, 4.0 Hz, 1H), 7.39 (d, J = 3.2 Hz, 1H), 7.23- 7.08 (m, 4H), 6.89 (dd, J = 4.0, 4.0 Hz, 1H), 6.68-6.66 (m, 2H), 5.99 (dd, J = 8.0, 1.2 Hz, 1H), 5.39-5.34 (m, 1H), 3.83 (d, J = 16.4 Hz, 1H), 3.43 (d, J = 16.4 Hz, 1H), 1.65-1.61 (m, 1H), 1.42-1.31 (m, 1H), 1.27 (d, J = 6.0 Hz, 3H),0.76-0.73 (m, 1H), 0.38-0.36 (m, 2H), 0.07- 0.00 (m, 2H). |
| 252 | SS | 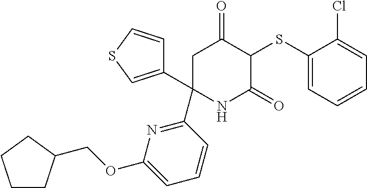 [SMILES: O=C1CC(c2ccsc2)(c2cccc(OCC3CCCC3)n2)NC(=O)C1Sc1ccccc1Cl] | 3-(2-chlorophenyl)sulfanyl- 6-[6-(cyclopentyl- methoxy)-2-pyridyl]-6- (3-thienyl)pipereidine- 2,4-dione was prepared as in example 187. | ¹H NMR (400 MHz, CD₃OD) δ 7.70 (dd, J = 8.0, 8.0 Hz, 1H), 7.41 (dd, J = 2.6, 2.6 Hz, 1H), 7.26 (d, J = 2.8 Hz, 1H), 7.20 (dd, J = 4.0, 4.0 Hz, 1H), 7.14-7.11 (m, 2H), 6.91 (dd, J = 8.0, 8.0 Hz, 1H), 6.73 (dd, J = 4.0, 4.0 Hz, 2H), 5.97 (d, J = 8.0 Hz, 1H), 4.27-4.17 (m, 2H), 3.84 (d, J = 16.0 Hz, 1H), 3.43 (d, J = 16.0 Hz, 1H), 2.34-2.27 (m, 1H), 1.78-1.75 (m, 2H), 1.61-1.53 (m, 4H), 1.34-1.31 (m, 2H). |
| 253 | SS | 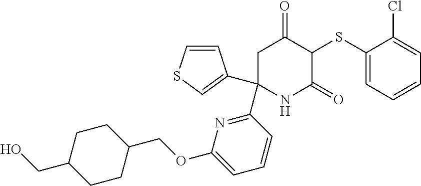 [SMILES: O=C1CC(c2ccsc2)(c2cccc(OCC3CCC(CO)CC3)n2)NC(=O)C1Sc1ccccc1Cl] | Prepared according to methods described therein. | ¹H NMR (400 MHz, CD₃OD) δ 7.70 (dd, J = 8.0, 8.0 Hz, 1H), 7.41 (dd, J = 5.2, 3.2 Hz, 1H), 7.25-7.12 (m, 4H), 6.94 (dd, J = 8.0, 8.0 Hz, 1H), 6.75-6.72 (m, 2H), 5.95 (dd, J = 8.0, 1.6 Hz, 1H), 4.25-4.21 (m, 1H), 4.15- 4.12 (m, 1H), 3.88 (d, J = 16.4 Hz, 1H), 3.43 (d, J = 16.4 Hz, 1H), 1.86-1.76 (m, 5H), 1.39- 1.36 (m, 1H), 1.06-0.86 (m, 4H). |
| 254 | SS | 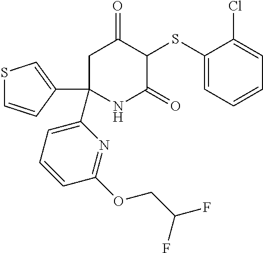 [SMILES: O=C1CC(c2ccsc2)(c2cccc(OCC(F)F)n2)NC(=O)C1Sc1ccccc1Cl] | 3-(2-chlorophenyl)sulfanyl- 6-[6-(2,2-difluoroethoxy)- 2-pyridyl]-6-(3-thienyl) piperidine-2,4-dione was prepared in 20% yield according to Example 2, Step A substituting propan-2-ol for 2,2- difluoroethanol. | ¹H NMR (400 MHz, (CD₃)₂SO) δ: 8.47 (s, 1H), 7.85 (dd, J = 8.0, 8.0 Hz, 1H), 7.49-7.48 (m, 1H), 7.35 (s, 1H), 7.30-7.27 (m, 2H), 7.17 (d, J = 3.6 Hz, 1H), 6.88 (d, J = 8.0 Hz, 1H), 6.87-6.86 (m, 1H), 6.71-6.70 (m, 1H), 6.33- 6.19 (m, 1H), 5.81 (d, J = 7.6 Hz, 1H), 4.64-4.49 (m, 1H), 3.87 (d, J = 16.4 Hz, 1H), 3.34 (d, J = 16.4 Hz, 1H). |
| 255 | SS | 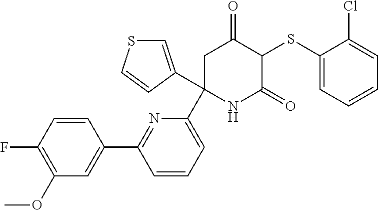 [SMILES: COc1cc(-c2cccc(C3(c4ccsc4)CC(=O)C(Sc4ccccc4Cl)C(=O)N3)n2)ccc1F] | 3-((2-chlorophenyl)thio)- 6-(6-(4-fluoro-3-methoxy- phenyl)pyridin-2-yl)-6- (thiophen-3-yl)piperidine- 2,4-dione was prepared in 51% yield according to the Example 418, substituting N,N-dimethyl- 4-(4,4,5,5-tetramethyl- 1,3,2-dioxaborolan-2-yl) benzenesulfonamide for 2- (4-fluoro-3-methoxy- phenyl)-4,4,5,5-tetramethyl- 1,3,2-dioxaborolane | ¹H NMR (400 MHz, DMSO) δ 11.82-11.57 (m, 1H), 8.45 (s, 1H), 8.04-7.85 (m, 3H), 7.82-7.71 (m, 1H), 7.62 (dd, J = 6.2, 2.4 Hz, 1H), 7.52 (dd, J = 5.1, 3.0 Hz, 1H), 7.40 (dd, J = 2.9, 1.4 Hz, 1H), 7.34-7.21 (m, 2H), 7.17 (dd, J = 5.1, 1.4 Hz, 1H), 6.87 (t, J = 7.6 Hz, 1H), 6.54 (t, J = 8.1 Hz, 1H), 5.87 (d, J = 7.5 Hz, 1H), 3.91 (d, J = 8.9 Hz, 3H), 3.43-3.32 (m, 2H). |
| 256 | SS | 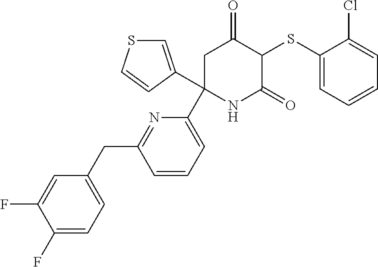 [SMILES: O=C1CC(c2ccsc2)(c2cccc(Cc3ccc(F)c(F)c3)n2)NC(=O)C1Sc1ccccc1Cl] | 3-((2-chlorophenyl)thio)- 6-(6-(3,4-difluorobenzyl) pyridin-2-yl)-6-(thiophen- 3-yl)piperidine-2,4-dione was prepared in 36.9% yield according to the Example 5, Step B substituting 1-(bromo- methyl)-3-fluorobenzene for 4-(bromomethyl)-1,2,- difluorobenzene | ¹H NMR (400 MHz, DMSO) δ 11.57 (s, 1H), 8.39 (s, 1H), 7.79 (t, J = 7.8 Hz, 1H), 7.56-7.47 (m, 2H), 7.38-7.06 (m, 7H), 6.91- 6.82 (m, 1H), 6.49 (t, J = 7.1 Hz, 1H), 5.69 (d, J = 7.8 Hz, 1H), 4.08 (s, 2H), 3.35 (d, J = 16.5 Hz, 2H). |
| 257 | SS | 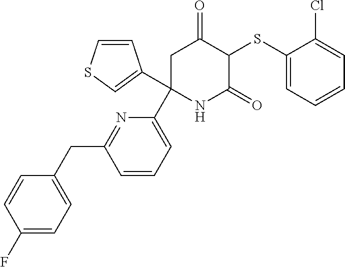 [SMILES: O=C1CC(c2ccsc2)(c2cccc(Cc3ccc(F)cc3)n2)NC(=O)C1Sc1ccccc1Cl] | 3-((2--chlorophenyl)thio)- 6-(6-(4-fluorobenzyl) pyridin-2-yl)-6-(thiophen- 3-yl)piperidine-2,4- dione was prepared in 13% yield according to the Example 5, Step B substituting 1-(bromo- methyl)-3-fluorobenzene for 1-(bromomethyl)-4- fluorobenzene | ¹H NMR (400 MHz, DMSO) δ 11.82-11.44 (m, 1H), 8.36-8.00 (m, 1H), 7.73 (t, J = 7.8 Hz, 1H), 7.50-7.41 (m, 2H), 7.36- 7.27 (m, 3H), 7.20 (t, J = 8.2 Hz, 2H), 7.09 (dd, J = 5.1, 1.4 Hz, 1H), 7.02 (t, J = 8.9 Hz, 2H), 6.84 (t, J = 7.6 Hz, 1H), 6.56 (t, J = 7.1 Hz, 1H), 5.87 (s, 1H), 4.08 (s, 2H), 3.26- 3.20 (m, 2H). |
| 258 | MD | 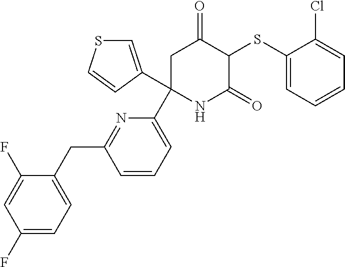 [SMILES: O=C1CC(c2ccsc2)(c2cccc(Cc3ccc(F)cc3F)n2)NC(=O)C1Sc1ccccc1Cl] | 3-((2-chlorophenyl)thio)- 6-(6-(2,4-difluorobenzyl) pyridin-2-yl)-6-(thiophen- 3-yl)piperidine-2,4-dione was prepared in 85.5% yield according to the Example 5, Step B substituting 1-(bromo- methyl)-3-fluorobenzene for 1-(bromomethyl)-2,4- difluorobenzene | ¹H NMR (400 MHz, DMSO) δ 11.79-11.40 (m, 1H), 7.78 (t, J = 7.8 Hz, 1H), 7.51-7.44 (m, 2H), 7.36 (dd, J = 15.5, 8.7 Hz, 2H), 7.30-7.23 (m, 2H), 7.20-7.12 (m, 2H), 7.05 (dd, J = 5.1, 1.3 Hz, 1H), 6.97-6.84 (m, 2H), 6.61 (t, J = 7.6 Hz, 1H), 5.83 (d, J = 7.8 Hz, 1H), 4.13 (t, J = 9.4 Hz, 2H), 3.76 (d, J = 15.1 Hz, 1H), 3.50 (dt, J = 25.6, 6.5 Hz, 1H). |
| 259 | MD | 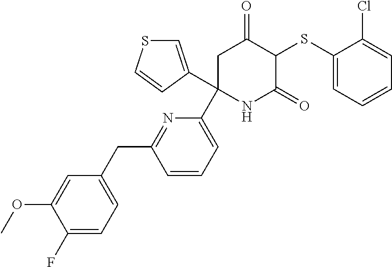 [SMILES: COc1cc(Cc2cccc(C3(c4ccsc4)CC(=O)C(Sc4ccccc4Cl)C(=O)N3)n2)ccc1F] | 3-((2-chlorophenyl)thio)- 6-(6-(4-fluoro-3-methoxy- benzyl)pyridin-2-yl)-6- (thiophen-3-yl)piperidine- 2,4-dione was prepared in 46.5% yield according to the Example 5, Step B substituting 1-(bromo- methyl)-3-fluorobenzene for 4-(bromomethyl)-1- fluoro-2-methoxybenzene | ¹H NMR (400 MHz, DMSO) δ 11.73-11.50 (m, 1H), 8.40-8.23 (m, 1H), 7.77 (t, J = 7.8 Hz, 1H), 7.53-7.45 (m, 2H), 7.31 (dd, J = 2.9, 1.4 Hz, 1H), 7.25 (d, J = 7.1 Hz, 2H), 7.11 (dd, J = 5.1, 1.4 Hz, 1H), 7.03 (ddd, J = 15.6, 8.9, 5.1 Hz, 2H), 6.92-6.80 (m, 2H), 6.54 (t, J = 7.7 Hz, 1H), 5.78 (d, J = 8.5 Hz, 1H), 4.07 (s, 2H), 3.90 (s, 1H), 3.72 (s, 3H), 3.36 (d, J = 16.4 Hz, 1H). |
| 260 | MD | 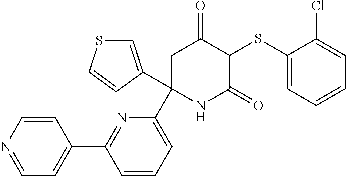 [SMILES: O=C1CC(c2ccsc2)(c2cccc(-c3ccncc3)n2)NC(=O)C1Sc1ccccc1Cl] | 6-([2,4′-bipyridin]-6-yl)-3- ((2-chlorophenyl)thio)-6- (thiophen-3-yl)piperidine- 2,4-dione was prepared in 15.1% yield according to Example 418, substituting N,N-dimethyl-4-(4,4,5,5- tetramethyl-1,3,2-dioxa- borolan-2-yl)benzene- sulfonamide for 4-(4,4,5,5- tetramethyl-1,3,2-dioxa- borolan-2-yl)pyridine | ¹H NMR (400 MHz, DMSO) δ 12.04-11.67 (m, 1H), 8.70 (dd, J = 4.5, 1.6 Hz, 2H), 8.18-8.07 (m, 3H), 8.02 (t, J = 7.8 Hz, 1H), 7.77 (d, J = 7.2 Hz, 1H), 7.49 (dd, J = 5.1, 2.9 Hz, 1H), 7.39 (dd, J = 2.9, 1.3 Hz, 1H), 7.19 (dd, J = 5.1, 1.3 Hz, 2H), 6.82 (t, J = 7.7 Hz, 1H), 6.52 (dd, J = 17.1, 9.4 Hz, 2H), 5.85 (s, 1H), 3.88 (s, 1H), 3.31 (s, 1H). |
| 261 | MD | 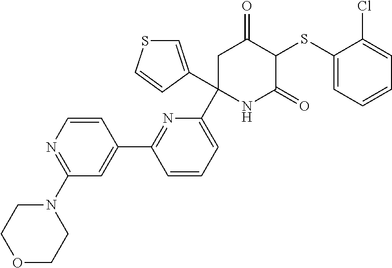 [SMILES: O=C1CC(c2ccsc2)(c2cccc(-c3ccnc(N4CCOCC4)c3)n2)NC(=O)C1Sc1ccccc1Cl] | 3-((2-chlorophenyl)thio)-6- (2′-morpholino-[2,4′- bipyridin]-6-yl)-6- (thiophen-3-yl)piperidine- 2,4-dione was prepared in 9.8% yield according to Example 418, substituting N,N-dimethyl-4-(4,4,5,5- tetramethyl-1,3,2-dioxa- borolan-2-yl)benzene- sulfonamide for 4-(4- (4,4,5,5-tetramethyl- 1,3,2-dioxaborolan-2- yl)pyridine-2-yl)morpholine | ¹H NMR (400 MHz, DMSO) δ 11.83-11.59 (m, 1H), 8.23 (d, J = 5.0 Hz, 1H), 8.07-7.96 (m, 2H), 7.71 (d, J = 6.8 Hz, 1H), 7.53-7.46 (m, 2H), 7.44-7.37 (m, 2H), 7.22 (d, J = 7.3 Hz, 1H), 7.16 (dd, J = 5.1, 1.4 Hz, 1H), 6.85 (t, J = 6.8 Hz, 1H), 6.52 (dd, J = 17.0, 10.2 Hz, 2H), 5.85 (s, 1H), 3.99-3.87 (m, 1H), 3.75-3.66 (m, 4H), 3.51 (dd, J = 8.3, 4.4 Hz, 4H), 3.32 (s, 1H). |
| 262 | MD | 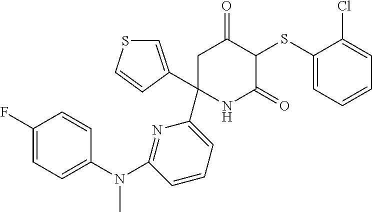 [SMILES: CN(c1ccc(F)cc1)c1cccc(C2(c3ccsc3)CC(=O)C(Sc3ccccc3Cl)C(=O)N2)n1] | 3-((2-chlorophenyl)thio)- 6-(6-((4-fluorophenyl) (methyl)amino)pyridin-2-yl)- 6-(thiophen-3-yl)piperidine- 2,4-dione was prepared in 10.2% yield according to the Example 4, Step A substituting cyclo- hexanamine for 4-fluoro- N-methylaniline | ¹H NMR (400 MHz, DMSO) δ 11.80-11.38 (m, 1H), 8.13 (s, 1H), 7.57-7.45 (m, 2H), 7.32 (dddd, J = 17.6, 13.5, 6.0, 1.9 Hz, 5H), 7.17 (dt, J = 12.6, 6.3 Hz, 1H), 7.01-6.90 (m, 2H), 6.77 (dd, J = 17.0, 8.9 Hz, 1H), 6.39 (d, J = 8.4 Hz, 1H), 6.03 (d, J = 8.0 Hz, 1H), 3.79 (d, J = 14.2 Hz, 1H), 3.40 (s, 3H), 3.31 (s, 2H). |
| 263 | SS | 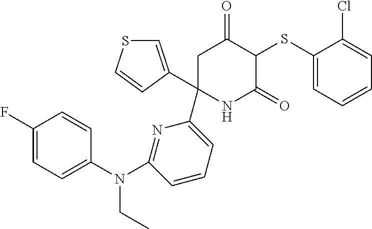 [SMILES: CCN(c1ccc(F)cc1)c1cccc(C2(c3ccsc3)CC(=O)C(Sc3ccccc3Cl)C(=O)N2)n1] | 3-((2-chlorophenyl)thio)- 6-(6-(ethyl(4-fluorophenyl) amino)pyridin-2-yl)-6- (thiophen-3-yl)piperidine- 2,4-dione was prepared in 11.7% yield according to the Example 4, Step A substituting cyclo- hexanamine for N-ethyl- 4-fluoroaniline | ¹H NMR (400 MHz, DMSO) δ 11.79-11.35 (m, 1H), 8.16 (d, J = 23.6 Hz, 1H), 7.51 (dd, J = 5.0, 3.0 Hz, 1H), 7.46-7.39 (m, 2H), 7.29 (d, J = 6.9 Hz, 5H), 7.19 (dd, J = 5.1, 1.4 Hz, 1H), 6.99-6.89 (m, 2H), 6.79-6.72 (m, 1H), 6.19 (d, J = 8.4 Hz, 1H), 6.03 (dd, J = 8.0, 1.3 Hz, 1H), 3.98 (dt, J = 14.1, 7.1 Hz, 1H), 3.85 (dt, J = 29.0, 10.4 Hz, 2H), 3.35 (s, 1H), 1.10 (t, J = 7.0 Hz, 3H). |
| 264 | SS | 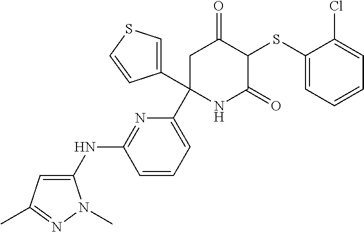 [SMILES: Cc1cc(Nc2cccc(C3(c4ccsc4)CC(=O)C(Sc4ccccc4Cl)C(=O)N3)n2)n(C)n1] | 3-((2-chlorophenyl)thio)- 6-(6-((1,3-dimethyl-1H- pyrazol-5-yl)amino) pyridin-2-yl)-6-(thiophen- 3-yl)piperidine-2,4- dione was prepared in 30.9% yield according to the Example 4, Step A substituting cyclo- hexanamine for 1,3- dimethyl-1H-pyrazol-5- amine | ¹H NMR (400 MHz, DMSO) δ 11.59-11.35 (m, 1H), 8.76 (s, 1H), 8.12-7.95 (m, 1H), 7.65- 7.60 (m, 1H), 7.50 (dd, J = 5.0, 3.0 Hz, 1H), 7.34 (dd, J = 3.0, 1.4 Hz, 1H), 7.28 (d, J = 6.8 Hz, 1H), 7.14 (dd, J = 5.1, 1.4 Hz, 1H), 7.01 (d, J = 7.3 Hz, 1H), 6.96 (t, J = 7.5 Hz, 1H), 6.80 (t, J = 7.0 Hz, 1H), 6.71 (d, J = 8.1 Hz, 1H), 6.08 (d, J = 6.7 Hz, 1H), 6.00 (s, 1H), 3.62 (s, 1H), 3.56 (s, 3H), 3.37 (d, J = 19.5 Hz, 1H), 2.10 (s, 3H). |
| 265 | SS | 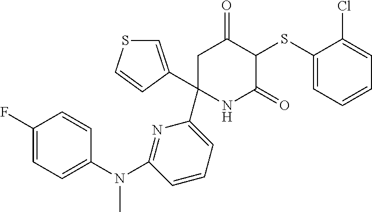 [SMILES: CN(c1ccc(F)cc1)c1cccc(C2(c3ccsc3)CC(=O)C(Sc3ccccc3Cl)C(=O)N2)n1] | 3-((2-chlorophenyl)thio)- 6-(6-((4-fluorophenyl) (methyl)amino)pyridin-2- yl)-6-(thiophen-3-yl) piperidine-2,4-dione was prepared in 7.8% yield according to the Example 4, Step A substituting cyclohexanamine for was prepared in 11.7% yield according to the Example 4, Step A substituting cyclohexanamine for N- ethyl-4-fluoroaniline | ¹H NMR (400 MHz, DMSO) δ 11.76-11.34 (m, 1H), 8.18 (s, 1H), 7.54-7.45 (m, 2H), 7.39- 7.23 (m, 5H), 7.18 (dd, J = 5.1, 1.4 Hz, 1H), 6.95 (dt, J = 8.3, 3.5 Hz, 2H), 6.80-6.72 (m, 1H), 6.40 (d, J = 8.3 Hz, 1H), 6.02 (dd, J = 8.0, 1.4 Hz, 1H), 3.83 (d, J = 15.4 Hz, 1H), 3.40 (s, 3H), 3.33 (s, 2H). |
| 266 | SS | 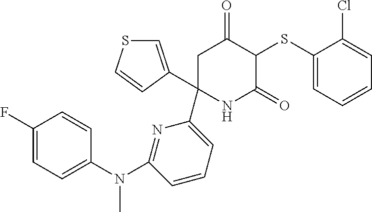 [SMILES: CN(c1ccc(F)cc1)c1cccc(C2(c3ccsc3)CC(=O)C(Sc3ccccc3Cl)C(=O)N2)n1] | 3-((2-chlorophenyl)thio)- 6-(6-((4-fluorophenyl) (methyl)amino)pyridin-2- yl)-6-(thiophen-3-yl) piperidine-2,4-dione was prepared in 7.1% yield according to the Example 4, Step A substituting cyclohexanamine for was prepared in 11.7% yield according to the Example 4, Step A substituting cyclohexanamine for N- ethyl-4-fluoroaniline | ¹H NMR (400 MHz, DMSO) δ 11.71-11.29 (m, 1H), 8.18-8.00 (m, 1H), 7.54-7.46 (m, 2H), 7.38 (dd, J = 3.0, 1.4 Hz, 1H), 7.34-7.25 (m, 4H), 7.18 (dd, J = 5.1, 1.4 Hz, 1H), 6.95 (t, J = 6.1 Hz, 2H), 6.79-6.72 (m, 1H), 6.39 (d, J = 8.3 Hz, 1H), 6.03 (d, J = 6.6 Hz, 1H), 3.78 (s, 1H), 3.40 (s, 3H), 3.32 (s, 2H). |
| 267 | MD | 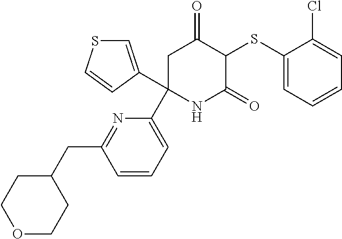 [SMILES: O=C1CC(c2ccsc2)(c2cccc(CC3CCOCC3)n2)NC(=O)C1Sc1ccccc1Cl] | 3-((2-chlorophenyl)thio)- 6-(6-((tetrahydro-2H-pyran- 4-yl)methyl)pyridin-2-yl)- 6-(thiophen-3-yl) piperidine-2,4-dione was prepared in 2.0% yield according to the Example 5, Step B substituting 1-(bromomethyl)-3-fluoro- benzene for 4-(bromo- methyl)tetrahydro-2H-pyran | m/z: 513.1 100% purity by UV 254 nm |
| 268 | MD | 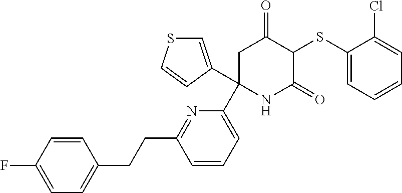 [SMILES: O=C1CC(c2ccsc2)(c2cccc(CCc3ccc(F)cc3)n2)NC(=O)C1Sc1ccccc1Cl] | 3-((2-chlorophenyl)thio)- 6-(6-(4-fluorophenethyl) pyridin-2-yl)-6-(thiophen- 3-yl)piperidine-2,4-dione was prepared in 7.1% yield according to the Example 5, Step B substituting 1- (bromomethyl)-3-fluoro- benzene for 1-(2-bromo- ethyl)-4-fluorobenzene | ¹H NMR (400 MHz, DMSO) δ 11.76-11.42 (m, 1H), 8.25-7.98 (m, 1H), 7.72 (t, J = 7.7 Hz, 1H), 7.50-7.43 (m, 2H), 7.30- 7.22 (m, 2H), 7.16-7.10 (m, 3H), 6.94 (ddd, J = 15.3, 8.8, 5.5 Hz, 3H), 6.71-6.66 (m, 1H), 5.88 (d, J = 7.6 Hz, 1H), 3.85 (s, 1H), 3.31 (s, 2H), 3.03 (dt, J = 11.8, 6.6 Hz, 4H). |
| 269 | MD | 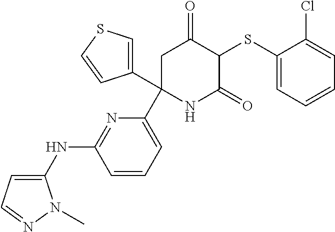 [SMILES: Cn1nccc1Nc1cccc(C2(c3ccsc3)CC(=O)C(Sc3ccccc3Cl)C(=O)N2)n1] | 3-((2-chlorophenyl)thio)- 6-(6-((1-methyl-1H- pyrazol-5-yl)amino) pyridin-2-yl)-6-(thiophen- 3-yl)piperidine-2,4-dione was prepared in 30.5% yield according to the Example 4, Step A substituting cyclo- hexanamine for 1-methyl- 1H-pyrazol-5-amine | ¹H NMR (400 MHz, DMSO) δ 11.67-11.35 (m, 1H), 8.85 (s, 1H), 7.63 (t, J = 7.9 Hz, 1H), 7.48 (dd, J = 5.0, 3.0 Hz, 1H), 7.36-7.30 (m, 2H), 7.27 (d, J = 8.0 Hz, 1H), 7.13 (dd, J = 5.1, 1.3 Hz, 1H), 7.03 (d, J = 7.4 Hz, 1H), 6.95 (t, J = 6.9 Hz, 1H), 6.85-6.71 (m, 2H), 6.27 (d, J = 1.9 Hz, 1H), 6.08 (d, J = 7.3 Hz, 1H), 3.65 (s, 4H), 3.32 (s, 2H). |
| 270 | MD | 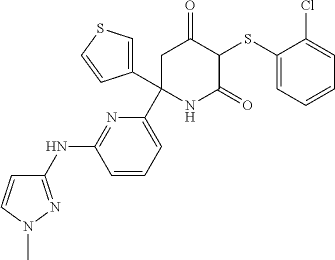 [SMILES: Cn1ccc(Nc2cccc(C3(c4ccsc4)CC(=O)C(Sc4ccccc4Cl)C(=O)N3)n2)n1] | 3-((2-chlorophenyl)thio)- 6-(6-((1-methyl-1H- pyrazol-3-yl)amino) pyridin-2-yl)-6-(thiophen- 3-yl)piperidine-2,4-dione was prepared in 24.3% yield according to the Example 4, Step A substituting cyclo- hexanamine for 1-methyl- 1H-pyrazol-3-amine | ¹H NMR (400 MHz, DMSO) δ 11.45-10.99 (m, 1H), 9.24 (s, 1H), 7.90-7.71 (m, 1H), 7.59- 7.53 (m, 1H), 7.50 (d, J = 2.2 Hz, 1H), 7.47 (dd, J = 5.1, 3.0 Hz, 1H), 7.36 (dd, J = 2.9, 1.3 Hz, 1H), 7.25 (d, J = 7.8 Hz, 1H), 7.18 (dd, J = 5.1, 1.3 Hz, 1H), 7.02 (d, J = 8.3 Hz, 1H), 6.92 (t, J = 8.5 Hz, 2H), 6.77 (t, J = 7.6 Hz, 1H), 6.35 (d, J = 2.2 Hz, 1H), 6.11 (d, J = 6.9 Hz, 1H), 3.73 (s, 3H), 3.61 (s, 1H). |
| 271 | MD | 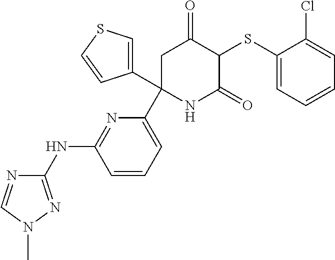 [SMILES: Cn1cnc(Nc2cccc(C3(c4ccsc4)CC(=O)C(Sc4ccccc4Cl)C(=O)N3)n2)n1] | 3-((2-chlorophenyl)thio)- 6-(6-((1-methyl-1H-1,2,4- triazol-3-yl)amino)pyridin- 2-yl)-6-(thiophen-3-yl) piperidine-2,4-dione was prepared in 20.8% yield according to the Example 4, Step A substituting cyclohexanamine for 1- methyl-1H-1,2,4-triazol- 3-amine | ¹H NMR (400 MHz, DMSO) δ 11.71-11.27 (m, 1H), 9.45 (s, 1H), 8.25 (s, 1H), 8.13 (s, 1H), 7.76-7.65 (m, 2H), 7.50 (dd, J = 5.1, 3.0 Hz, 1H), 7.41 (dd, J = 3.0, 1.4 Hz, 1H), 7.34- 7.23 (m, 2H), 7.03 (dd, J = 7.0, 1.2 Hz, 1H), 6.94 (td, J = 7.6, 1.5 Hz, 1H), 6.84- 6.74 (m, 1H), 6.05 (dd, J = 8.0, 1.4 Hz, 1H), 3.85-3.78 (m, 3H), 3.70 (d, J = 15.7 Hz, 1H), 3.42 (d, J = 15.9 Hz, 1H). |
| 272 | MD | 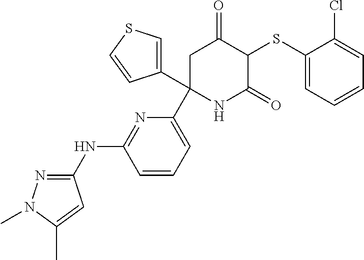 [SMILES: Cc1cc(Nc2cccc(C3(c4ccsc4)CC(=O)C(Sc4ccccc4Cl)C(=O)N3)n2)nn1C] | 3-((2-chlorophenyl)thio)- 6-(6-((1,5-dimethyl-1H- pyrazol-3-yl)amino)pyridin- 2-yl)-6-(thiophen-3-yl) piperidine-2,4-dione was prepared in 39.9% yield according to the Example 4, Step A substituting cyclohexanamine for 1,5- dimethyl-1H-pyrazol- 3-amine | ¹H NMR (400 MHz, DMSO) δ 11.58-11.34 (m, 1H), 9.11 (s, 1H), 8.19 (s, 1H), 7.61-7.54 (m, 1H), 7.51 (dd, J = 5.0, 3.0 Hz, 1H), 7.38 (dd, J = 3.0, 1.4 Hz, 1H), 7.29 (dd, J = 7.9, 1.2 Hz, 1H), 7.19 (dd, J = 5.1, 1.4 Hz, 1H), 7.07 (d, J = 8.3 Hz, 1H), 6.95 (ddd, J = 21.6, 10.6, 4.5 Hz, 2H), 6.83-6.77 (m, 1H), 6.07 (dd, J = 8.8, 2.2 Hz, 2H), 3.75 (d, J = 16.1 Hz, 1H), 3.60 (s, 3H), 3.43 (d, J = 16.6 Hz, 1H), 2.21 (s, 3H). |
| 273 | MD | 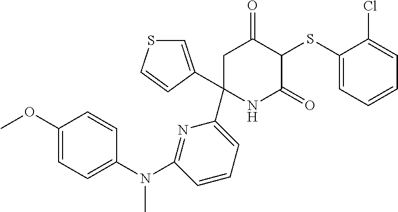 [SMILES: COc1ccc(N(C)c2cccc(C3(c4ccsc4)CC(=O)C(Sc4ccccc4Cl)C(=O)N3)n2)cc1] | 3-((2-chlorophenyl)thio)- 6-(6-((4-methoxyphenyl) (methyl)amino)pyridin-2- yl)-6-(thiophen-3-yl) piperidine-2,4-dione was prepared in 20% yield according to the Example 4, Step A substituting cyclo- hexanamine for 4- methoxy-N-methylaniline | ¹H NMR (400 MHz, DMSO) δ 11.67-11.41 (m, 1H), 7.89-7.80 (m, 2H), 7.49 (dd, J = 5.0, 3.0 Hz, 1H), 7.45-7.38 (m, 2H), 7.21-7.19 (m, 2H), 7.13-7.10 (m, 2H), 7.03-6.97 (m, 2H), 6.88 (d, J = 7.3 Hz, 1H), 6.74 (dd, J = 11.2, 4.1 Hz, 1H), 6.27 (d, J = 8.5 Hz, 1H), 6.04 (d, J = 7.8 Hz, 1H), 4.02 (s, 1H), 3.86 (s, 3H), 3.78 (s, 3H), 3.17 (d, J = 10.0 Hz, 1H). |
| 274 | MD | 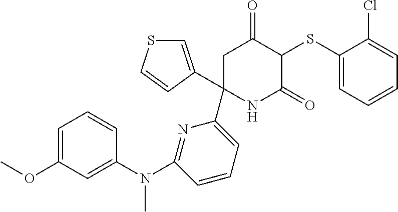 [SMILES: COc1cccc(N(C)c2cccc(C3(c4ccsc4)CC(=O)C(Sc4ccccc4Cl)C(=O)N3)n2)c1] | 3-((2-chlorophenyl)thio)- 6-(6-((3-methoxyphenyl) (methyl)amino)pyridin-2- yl)-6-(thiophen-3-yl) piperidine-2,4-dione was prepared in 22.7% yield according to the Example 4, Step A substituting cyclohexanamine for 3- methoxy-N-methylaniline | ¹H NMR (400 MHz, DMSO) δ 12.12-11.49 (m, 1H), 8.04 (d, J = 8.1 Hz, 1H), 7.74 (t, J = 7.9 Hz, 1H), 7.53-7.37 (m, 3H), 7.27-7.15 (m, 3H), 6.95- 6.87 (m, 2H), 6.84-6.73 (m, 3H), 6.50-6.47 (m, 1H), 6.07 (dd, J = 20.5, 8.2 Hz, 1H), 4.02 (t, J = 9.1 Hz, 1H), 3.82-3.69 (m, 3H), 3.46 (d, J = 36.8 Hz, 3H), 3.16 (t, J = 8.0 Hz, 1H). |
| 275 | MD | 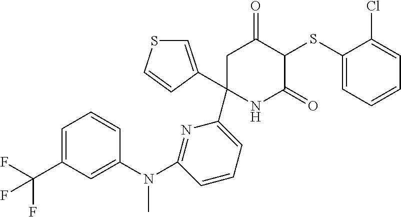 [SMILES: CN(c1cccc(C(F)(F)F)c1)c1cccc(C2(c3ccsc3)CC(=O)C(Sc3ccccc3Cl)C(=O)N2)n1] | 3-((2-chlorophenyl)thio)- 6-(6-(methyl(3- (trifluoromethyl)phenyl) amino)pyridin-2-yl)-6- (thiophen-3-yl)piperidine- 2,4-dione was prepared in 20.9% yield according to the Example 4, Step A substituting cyclo- hexanamine for N-methyl- 3-(trifluoromethyl)aniline | ¹H NMR (400 MHz, DMSO) δ 11.69-11.39 (m, 1H), 8.13 (s, 1H), 7.65-7.57 (m, 4H), 7.55- 7.48 (m, 2H), 7.37 (dd, J = 3.0, 1.4 Hz, 1H), 7.28 (d, J = 8.0 Hz, 1H), 7.16 (dd, J = 5.1, 1.4 Hz, 1H), 7.06 (d, J = 7.4 Hz, 1H), 6.94 (t, J = 6.8 Hz, 1H), 6.75 (dd, J = 11.3, 4.0 Hz, 1H), 6.64 (d, J = 8.3 Hz, 1H), 6.00 (d, J = 6.8 Hz, 1H), 4.12-3.94 (m, 1H), 3.78 (s, 1H), 3.47 (s, 3H). |
| 276 | MD | 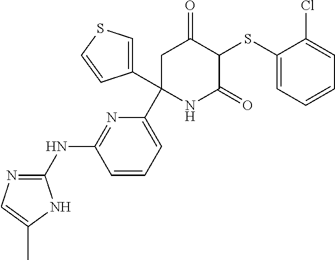 [SMILES: Cc1cnc(Nc2cccc(C3(c4ccsc4)CC(=O)C(Sc4ccccc4Cl)C(=O)N3)n2)[nH]1] | 3-((2-chlorophenyl)thio)- 6-(6-((5-methyl-1H- imidazol-2-yl)amino) pyridin-2-yl)-6-(thiophen- 3-yl)piperidine-2,4-dione was prepared in 2.2% yield according to the Example 4, Step A substituting cyclo- hexanamine for 5-methyl- 1H-imidazol-2-amine | m/z: 510.1 100% purity by UV 254 nm |
| 277 | MD | 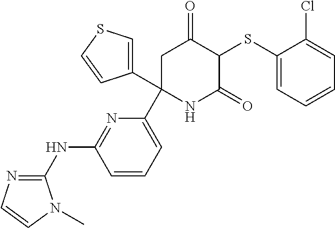 [SMILES: Cn1ccnc1Nc1cccc(C2(c3ccsc3)CC(=O)C(Sc3ccccc3Cl)C(=O)N2)n1] | 3-((2-chlorophenyl)thio)- 6-(6-((1-methyl-1H- imidazol-2-yl)amino) pyridin-2-yl)-6-(thiophen- 3-yl)piperidine-2,4-dione was prepared in 12.2% yield according to the Example 4, Step A substituting cyclo- hexanamine for 1-methyl- 1H-imidazol-2-amine | m/z: 510.1 91.5% purity by UV 254 nm |
| 278 | MD | 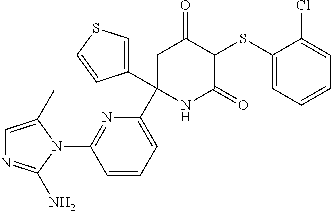 [SMILES: Cc1cnc(N)n1-c1cccc(C2(c3ccsc3)CC(=O)C(Sc3ccccc3Cl)C(=O)N2)n1] | 6-(6-(2-amino-5-methyl- 1H-imidazol-1-yl)pyridin- 2-yl)-3-((2-chloro- phenyl)thio)-6-(thiophen- 3-yl)piperidine-2,4-dione was prepared in 3.2% yield according to the Example 4, Step A substituting cyclo- hexanamine for 5-methyl- 1H-imidazol-2-amine | m/z: 510.1 96.5% purity by UV 254 nm |
| 279 | MD | 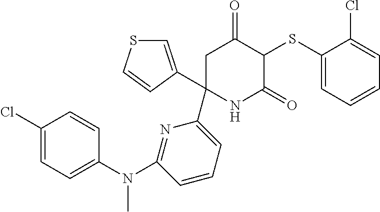 [SMILES: CN(c1ccc(Cl)cc1)c1cccc(C2(c3ccsc3)CC(=O)C(Sc3ccccc3Cl)C(=O)N2)n1] | 6-(6-((4-chlorophenyl) (methyl)amino)pyridin-2- yl)-3-((2-chlorophenyl) thio)-6-(thiophen-3-yl) piperidine-2,4-dione was prepared in 21.2% yield according to the Example 4, Step A substituting cyclohexanamine for 4- chloro-N-methylaniline | ¹H NMR (400 MHz, DMSO) δ 11.72-11.47 (m, 1H), 8.13 (s, 1H), 7.60-7.42 (m, 4H), 7.37 (dd, J = 3.0, 1.4 Hz, 1H), 7.36- 7.24 (m, 3H), 7.17 (dd, J = 5.1, 1.4 Hz, 1H), 7.02-6.93 (m, 2H), 6.75 (t, J = 7.7 Hz, 1H), 6.56 (t, J = 5.8 Hz, 1H), 6.00 (d, J = 6.9 Hz, 1H), 3.41 (s, 3H), 3.31 (s, 2H). |
| 280 | SS | 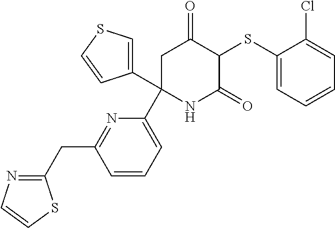 [SMILES: O=C1CC(c2ccsc2)(c2cccc(Cc3nccs3)n2)NC(=O)C1Sc1ccccc1Cl] | 3-((2-chlorophenyl)thio)- 6-(6-(thiazol-2-ylamino) pyridin-2-yl)-6-(thiophen- 3-yl)piperidine-2,4-dione was prepared in 11% yield according to the Example 4, Step 4 substituting cyclohexanamine for thiazol-2-amine | ¹H NMR (400 MHz, DMSO) δ 11.32 (s, 1H), 8.14 (s, 1H), 7.72 (t, J = 7.9 Hz, 1H), 7.49-7.43 (m, 2H), 7.39 (d, J = 3.6 Hz, 1H), 7.23 (ddd, J = 19.8, 8.4, 4.7 Hz, 3H), 7.05 (d, J = 3.6 Hz, 1H), 6.95 (d, J = 8.1 Hz, 1H), 6.88 (d, J = 7.7 Hz, 1H), 6.73 (t, J = 7.2 Hz, 1H), 6.54 (s, 1H), 6.05 (d, J = 8.0 Hz, 1H), 3.90-3.72 (m, 1H), 3.31-3.29 (m, 1H). |
| 281 | SS | 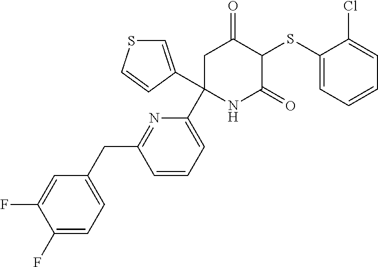 [SMILES: O=C1CC(c2ccsc2)(c2cccc(Cc3ccc(F)c(F)c3)n2)NC(=O)C1Sc1ccccc1Cl] | 3-((2-chlorophenyl)thio)- 6-(6-(3,4-difluorobenzyl) pyridin-2-yl)-6-(thiophen- 3-yl)piperidine-2,4-dione was prepared in 7.2% yield according to the Example 5, Step B substituting 1- (bromomethyl)-3-fluoro- benzene for (3,4-difluoro- phenyl)methanamine | ¹H NMR (400 MHz, DMSO) δ 11.77-11.53 (m, 1H), 8.35-8.10 (m, 1H), 7.78 (t, J = 7.8 Hz, 1H), 7.55-7.44 (m, 2H), 7.36- 7.06 (m, 7H), 6.85 (t, J = 6.9 Hz, 1H), 6.49 (t, J = 7.2 Hz, 1H), 5.72 (s, 1H), 4.08 (s, 2H), 3.90-3.78 (m, 1H), 3.30-3.26 (m, 1H). |
| 282 | MD | 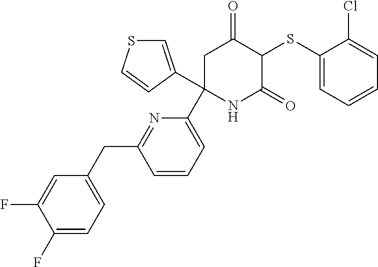 [SMILES: O=C1CC(c2ccsc2)(c2cccc(Cc3ccc(F)c(F)c3)n2)NC(=O)C1Sc1ccccc1Cl] | 3-((2-chlorophenyl)thio)- 6-(6-(3,4-difluorobenzyl) pyridin-2-yl)-6-(thiophen- 3-yl)piperidine-2,4-dione was prepared in 7.2% yield according to the Example 5, Step B substituting 1- (bromomethyl)-3-fluoro- benzene for (3,4-difluoro- phenyl)methanamine | ¹H NMR (400 MHz, DMSO) δ 11.84-11.40 (m, 1H), 8.35-8.05 (m, 1H), 7.77 (t, J = 7.8 Hz, 1H), 7.58-7.42 (m, 2H), 7.40- 7.03 (m, 7H), 6.84 (dd, J = 10.8, 4.5 Hz, 1H), 6.49 (t, J = 7.4 Hz, 1H), 5.73 (s, 1H), 4.08 (s, 2H), 3.81 (s, 1H), 3.27 (s, 1H). |
| 283 | MD | 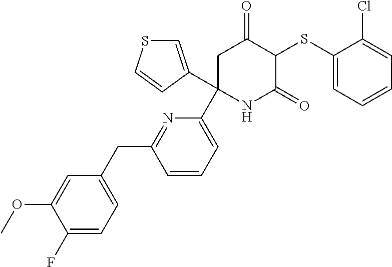 [SMILES: COc1cc(Cc2cccc(C3(c4ccsc4)CC(=O)C(Sc4ccccc4Cl)C(=O)N3)n2)ccc1F] | 3-((2-chlorophenyl)thio)- 6-(6-(4-fluoro-3-methoxy- benzyl)pyridin-2-yl)- 6-(thiophen-3-yl)piperidine- 2,4-dione was prepared in 8.9% yield according to the Example 5, Step B substituting 1-(bromo- methyl)-3-fluorobenzene for (4-fluoro-3-methoxy- phenyl)methanamine | ¹H NMR (400 MHz, DMSO) δ 11.91-11.41 (m, 1H), 8.31 (s, 1H), 7.77 (t, J = 7.8 Hz, 1H), 7.54-7.42 (m, 2H), 7.36-7.16 (m, 3H), 7.15-6.98 (m, 3H), 6.92-6.81 (m, 2H), 6.52 (t, J = 7.1 Hz, 1H), 5.76 (d, J = 7.7 Hz, 1H), 4.07 (s, 2H), 3.89 (s, 1H), 3.71 (s, 3H), 3.37 (s, 1H). |
| 284 | MD | 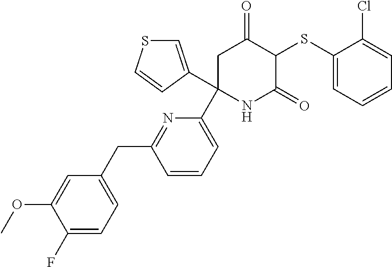 [SMILES: COc1cc(Cc2cccc(C3(c4ccsc4)CC(=O)C(Sc4ccccc4Cl)C(=O)N3)n2)ccc1F] | 3-((2-chlorophenyl)thio)- 6-(6-(4-fluoro-3-methoxy- benzyl)pyridin-2-yl)- 6-(thiophen-3-yl)piperidine- 2,4-dione was prepared in 10.7% yield according to the Example 5, Step B substituting 1-(bromo- methyl)-3-fluorobenzene for (4-fluoro-3-methoxy- phenyl)methanamine | ¹H NMR (400 MHz, DMSO) δ 11.82-11.47 (m, 1H), 8.32 (s, 1H), 7.78 (t, J = 7.8 Hz, 1H), 7.54-7.44 (m, 2H), 7.31 (dd, J = 3.0, 1.4 Hz, 1H), 7.25 (d, J = 7.8 Hz, 2H), 7.11 (dd, J = 5.1, 1.4 Hz, 1H), 7.03 (ddd, J = 14.9, 8.5, 5.1 Hz, 2H), 6.86 (ddd, J = 12.7, 6.8, 1.7 Hz, 2H), 6.52 (t, J = 7.1 Hz, 1H), 5.76 (d, J = 7.4 Hz, 1H), 4.07 (s, 2H), 3.89 (s, 1H), 3.71 (s, 3H), 3.37 (s, 1H). |
| 285 | MD | 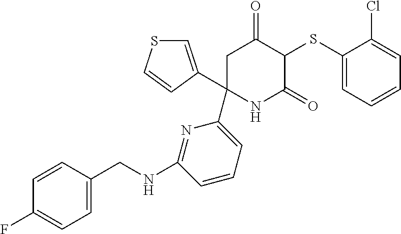 [SMILES: O=C1CC(c2ccsc2)(c2cccc(NCc3ccc(F)cc3)n2)NC(=O)C1Sc1ccccc1Cl] | 3-((2-chlorophenyl)thio)- 6-(6-((4-fluorobenzyl) amino)pyridin-2-yl)-6- (thiophen-3-yl)piperidine- 2,4-dione was prepared in 59.6% yield according to the Example 4, Step A substituting cyclo- hexanamine for (4-fluoro- phenyl)methanamine | ¹H NMR (400 MHz, DMSO) δ 11.65-11.37 (m, 1H), 8.13 (s, 1H), 7.42-7.33 (m, 4H), 7.30-7.14 (m, 3H), 7.12- 7.03 (m, 2H), 6.99-6.89 (m, 2H), 6.81-6.70 (m, 2H), 6.44 (d. J = 8.2 Hz, 1H), 6.02 (d, J = 6.9 Hz, 1H), 4.61-4.54 (m, 1H), 4.38 (dd, J = 15.3, 5.8 Hz, 1H), 3.78 (s, 1H), 3.20 (d, J = 16.4 Hz, 1H). |
| 286 | MD | 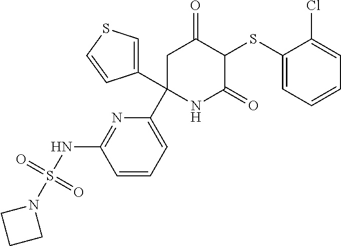 [SMILES: O=C1CC(c2ccsc2)(c2cccc(NS(=O)(=O)N3CCC3)n2)NC(=O)C1Sc1ccccc1Cl] | N-(6-(5-((2-chlorophenyl) thio)-4,6-dioxo-2- (thiophen-3-yl)piperidin- 2-yl)pyridin-2-yl)azetidine- 1-sulfonamide was prepared in 7% yield according to the Example 4, Step A substituting cyclo- hexanamine for azetidine- 1-sulfonamide | m/z: 549.1 94% purity by UV 254 nm |
| 287 | MD | 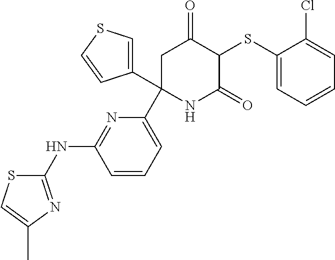 [SMILES: Cc1csc(Nc2cccc(C3(c4ccsc4)CC(=O)C(Sc4ccccc4Cl)C(=O)N3)n2)n1] | 3-((2-chlorophenyl)thio)- 6-(6-((4-methylthiazol- 2-yl)amino)pyridin-2-yl)-6- (thiophen-3-yl)piperidine- 2,4-dione was prepared in 59.1% yield according to the Example 4, Step A substituting cyclo- hexanamine for 4- methylthiazol-2-amine | ¹H NMR (400 MHz, DMSO) δ 11.22 (s, 1H), 7.71 (dd, J = 23.2, 15.5 Hz, 2H), 7.58 (s, 1H), 7.49- 7.40 (m, 2H), 7.21 (ddd, J = 29.8, 13.3, 4.6 Hz, 3H), 6.89 (dd, J = 13.9, 7.8 Hz, 2H), 6.77-6.70 (m, 1H), 6.59 (d, J = 1.0 Hz, 1H), 6.08 (d, J = 7.0 Hz, 1H), 3.73 (s, 2H), 2.21 (t, J = 13.6 Hz, 3H). |
| 288 | MD | 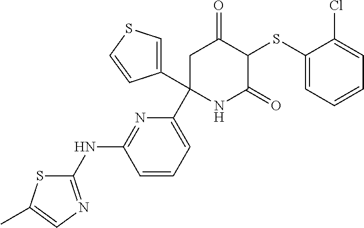 [SMILES: Cc1cnc(Nc2cccc(C3(c4ccsc4)CC(=O)C(Sc4ccccc4Cl)C(=O)N3)n2)s1] | 3-((2-chlorophenyl)thio)- 6-(6-((5-methylthiazol- 2-yl)amino)pyridin-2-yl)-6- (thiophen-3-yl)piperidine- 2,4-dione was prepared in 7.5% yield according to the Example 4, Step A substituting cyclo- hexanamine for 5- methylthiazol-2-amine | ¹H NMR (400 MHz, DMSO) δ 11.82-11.53 (m, 1H), 11.23- 10.94 (m, 1H), 8.37-8.13 (m, 1H), 7.85 (t, J = 7.8 Hz, 1H), 7.72 (t, J = 7.8 Hz,1H), 7.63 (d, J = 7.7 Hz, 1H), 7.60-7.41 (m, 2H), 7.34 (dd, J = 2.9, 1.4 Hz, 1H), 7.29-7.25 (m, 1H), 7.20-7.13 (m, 1H), 6.95 (d, J = 7.1 Hz, 1H), 6.79 (t, J = 7.7 Hz, 1H), 5.94 (d, J = 7.9 Hz, 1H), 3.74 (d, J = 25.7 Hz, 1H), 3.35 (s, 1H), 2.51 (s, 3H). |
| 289 | MD | 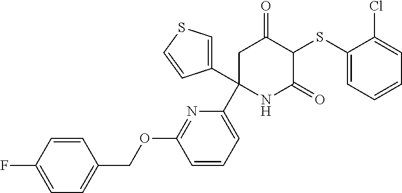 [SMILES: O=C1CC(c2ccsc2)(c2cccc(OCc3ccc(F)cc3)n2)NC(=O)C1Sc1ccccc1Cl] | 3-((2-chlorophenyl)thio)- 6-(6-((4-fluorobenzyl)oxy) pyridin-2-yl)-6-(thiophen- 3-yl)piperidine-2,4-dione was prepared in 35.6% yield according to the Example 2, Step A substituting propan-2-ol for (4-fluorophenyl)methanol | ¹H NMR (400 MHz, DMSO) δ 11.82-11.47 (m, 1H), 8.35 (s, 1H), 7.84-7.73 (m, 1H), 7.52- 7.40 (m, 3H), 7.28 (ddd, J = 13.9, 8.1, 4.3 Hz, 3H), 7.15 (ddd, J = 8.9, 5.8, 2.5 Hz, 2H), 7.06 (dd, J = 5.1, 1.4 Hz, 1H), 6.95 (td, J = 7.7, 1.5 Hz, 1H), 6.82 (d, J = 8.2 Hz, 1H), 6.77-6.69 (m, 1H), 5.91 (d, J = 6.9 Hz, 1H), 5.45 (d, J = 12.4 Hz, 1H), 5.31 (d, J = 12.4 Hz, 1H), 3.89 (d, J = 16.1 Hz, 1H), 3.34 (s, 1H). |
| 290 | MD | 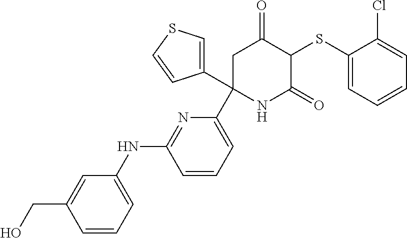 [SMILES: O=C1CC(c2ccsc2)(c2cccc(Nc3cccc(CO)c3)n2)NC(=O)C1Sc1ccccc1Cl] | 3-((2-chlorophenyl)thio)- 6-(6-((3-hydroxymethyl) phenyl)amino)pyridin-2-yl)- 6-(thiophen-3-yl)piperidine- 2,4-dione was prepared in 25.8% yield according to the Example 4, Step A substituting cyclo- hexanamine for (3-amino- phenyl)methanol | ¹H NMR (400 MHz, DMSO) δ 11.58-11.21 (m, 1H), 9.10 (s, 1H), 8.28 (s, 1H), 7.70 (s, 1H), 7.65- 7.58 (m, 1H), 7.51 (dd, J = 5.1, 3.0 Hz, 1H), 7.48-7.39 (m, 2H), 7.29 (dd, J = 7.9, 1.3 Hz, 1H), 7.26-7.17 (m, 2H), 7.05-6.94 (m, 2H), 6.87-6.73 (m, 3H), 6.07 (dd, J = 8.0, 1.4 Hz, 1H), 5.51- 5.24 (m, 1H), 4.50 (s, 2H), 3.83 (d, J = 16.0 Hz, 1H), 3.44 (d, J = 16.2 Hz, 1H). |
| 291 | SS | 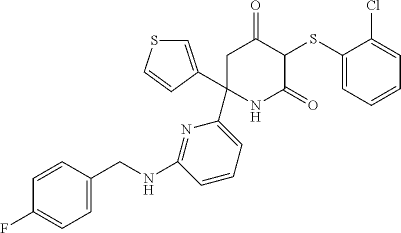 [SMILES: O=C1CC(c2ccsc2)(c2cccc(NCc3ccc(F)cc3)n2)NC(=O)C1Sc1ccccc1Cl] | 3-((2-chlorophenyl)thio)- 6-(6-((4-fluorobenzyl) amino)pyridin-2-yl)-6- (thiophen-3-yl)piperidine- 2,4-dione was prepared in 12% yield according to the Example 4, Step A substituting cyclo- hexanamine for (4-fluoro- phenyl)methanamine | ¹H NMR (400 MHz, DMSO) δ 11.56-11.31 (m, 1H), 8.10 (s, 1H), 7.43-7.32 (m, 4H), 7.28 (dd, J = 7.9, 1.2 Hz, 1H), 7.24- 7.17 (m, 2H), 7.07 (ddd, J = 8.9, 5.8, 2.6 Hz, 2H), 6.99-6.91 (m, 2H), 6.80-6.70 (m, 2H), 6.44 (d, J = 8.2 Hz, 1H), 6.03 (dd, J = 8.0, 1.4 Hz, 1H), 4.58 (dd, J = 15.1, 5.8 Hz, 1H), 4.39 (dd, J = 15.2, 5.5 Hz, 1H), 3.81 (d, J = 16.5 Hz, 1H), 3.22 (d, J = 15.9 Hz, 1H). |
| 292 | SS | 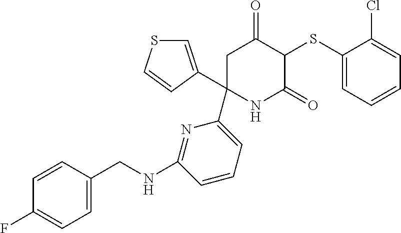 [SMILES: O=C1CC(c2ccsc2)(c2cccc(NCc3ccc(F)cc3)n2)NC(=O)C1Sc1ccccc1Cl] | 3-((2-chlorophenyl)thio)- 6-(6-((4-fluorobenzyl) amino)pyridin-2-yl)-6- (thiophen-3-yl)piperidine- 2,4-dione was prepared in 11% yield according to the Example 4, Step A substituting cyclo- hexanamine for (4-fluoro- phenyl)methanamine | ¹H NMR (400 MHz, DMSO) δ 11.60-11.34 (m, 1H), 8.15-7.99 (m, 1H), 7.43-7.32 (m, 4H), 7.28 (dd, J = 7.9, 1.2 Hz, 1H), 7.23-7.17 (m, 2H), 7.10-7.02 (m, 2H), 6.99-6.89 (m,2H), 6.81- 6.70 (m, 2H), 6.44 (d, J = 8.1 Hz, 1H), 6.04 (d, J = 6.7 Hz, 1H), 4.58 (dd, J = 14.9, 5.8 Hz, 1H), 4.39 (dd, J = 15.0, 5.5 Hz, 1H), 3.77 (s, 1H), 3.21 (d, J = 16.0 Hz, 1H). |
| 293 | SS | 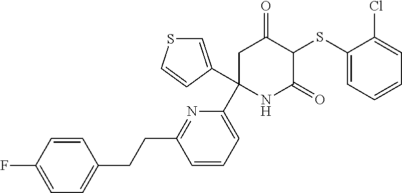 [SMILES: O=C1CC(c2ccsc2)(c2cccc(CCc3ccc(F)cc3)n2)NC(=O)C1Sc1ccccc1Cl] | 3-((2-chlorophenyl)thio)- 6-(6-(4-fluorophenethyl) pyridin-2-yl)-6-(thiophen- 3-yl)piperidine-2,4-dione was prepared in 13% yield according to the Example 5, Step B substituting 1-(bromo- methyl)-3-fluorobenzene for 1-(2-bromoethyl)-4- fluorobenzene | ¹H NMR (400 MHz, DMSO) δ 11.92-11.37 (m, 1H), 8.15-7.93 (m, 1H), 7.71 (t,J = 7.7 Hz, 1H), 7.50-7.43 (m, 2H), 7.26 (ddd, J = 9.1, 5.4, 1.3 Hz, 2H), 7.17-7.08 (m, 4H), 6.98-6.87 (m, 3H), 6.71-6.66 (m,1H), 5.89 (d, J = 7.8 Hz, 1H), 3.81 (s, 1H), 3.33 (s, 1H), 3.09-2.97 (m, 4H). |
| 294 | SS | 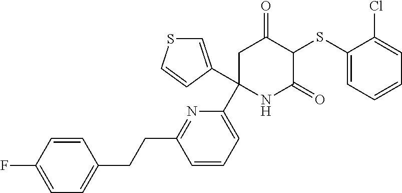 [SMILES: O=C1CC(c2ccsc2)(c2cccc(CCc3ccc(F)cc3)n2)NC(=O)C1Sc1ccccc1Cl] | 3-((2-chlorophenyl)thio)- 6-(6-(4-fluorophenethyl) pyridin-2-yl)-6-(thiophen- 3-yl)piperidine-2,4-dione was prepared in 12% yield according to the Example 5, Step B substituting 1-(bromo- methyl)-3-fluorobenzene for 1-(2-bromoethyl)-4- fluorobenzene | ¹H NMR (400 MHz, DMSO) δ 11.88-11.29 (m, 1H), 8.22-7.96 (m, 1H), 7.71 (t, J = 7.7 Hz, 1H), 7.47 (d, J = 7.1 Hz, 2H), 7.25 (d, J = 9.1 Hz, 2H), 7.12 (dd, J = 10.9, 6.0 Hz, 4H), 6.94 (dd, J = 15.0, 6.4 Hz, 3H), 6.69 (d, J = 7.5 Hz, 1H), 5.87 (s, 1H), 3.83 (s, 1H), 3.32 (s, 1H), 3.04 (d, J = 10.1 Hz, 4H). |
| 295 | SS | 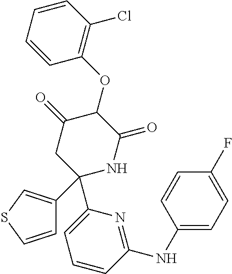 [SMILES: O=C1CC(c2ccsc2)(c2cccc(Nc3ccc(F)cc3)n2)NC(=O)C1Oc1ccccc1Cl] | 3-(2-chlorophenoxy)-6-[6- (4-fluoroanilino)-2-pyridyl]- 6-(3-thienyl)piperidine- 2,4-dione was prepared according to methods described in example 422. | |
| 296 | SS | 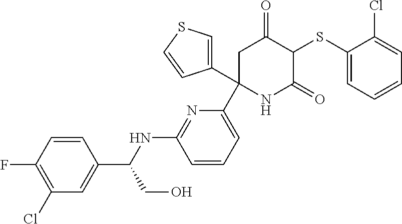 [SMILES: O=C1CC(c2ccsc2)(c2cccc(N[C@H](CO)c3ccc(F)c(Cl)c3)n2)NC(=O)C1Sc1ccccc1Cl] | 6-(6-(((S)-1-(3-chloro-4- fluorophenyl)-2-hydroxy- ethyl)amino)pyridin-2- yl)-3-((2-chlorophenyl) thio)-6-(thiophen-3-yl) piperidine-2,4-dione was prepared in 12.3% yield according to the Example 4, Step A substituting cyclohexanamine for (S)-2-amino-2-(4-fluoro- phenyl)ethanol | ¹H NMR (400 MHz, DMSO) δ 11.65-11.17 (m, 1H), 8.17 (d, J = 32.5 Hz, 1H), 7.84 (s, 1H), 7.61-7.52 (m, 1H), 7.45-7.34 (m, 3H), 7.33-7.23 (m, 2H), 7.23-7.09 (m, 1H), 7.07-6.87 (m, 3H), 6.81-6.66 (m, 2H), 6.60 (dd, J = 26.8, 6.4 Hz, 1H), 6.47 (d, J = 8.4 Hz, 1H), 5.99 (dd, J = 15.2, 8.1 Hz, 1H), 5.08- 4.83 (m, 2H), 3.65 (s, 2H). |
| 297 | SS | 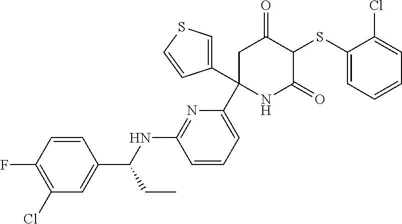 [SMILES: CC[C@@H](Nc1cccc(C2(c3ccsc3)CC(=O)C(Sc3ccccc3Cl)C(=O)N2)n1)c1ccc(F)c(Cl)c1] | 6-(6-(((R)-1-(3-chloro-4- fluorophenyl)propyl) amino)pyridin-2-yl)-3- ((2-chlorophenyl)thio)-6- (thiophen-3-yl)piperidine- 2,4-dione was prepared in 4.1% yield according to the Example 4, Step A substituting cyclo- hexanamine for (R)-1-(4- fluorophenyl)propan- 1-amine | m/z: 600.1 100% purity by UV 254 nm |
| 298 | SS | 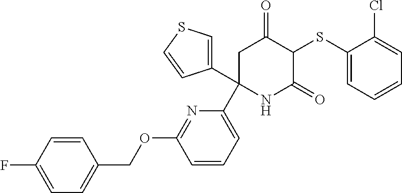 [SMILES: O=C1CC(c2ccsc2)(c2cccc(OCc3ccc(F)cc3)n2)NC(=O)C1Sc1ccccc1Cl] | 3-((2-chlorophenyl)thio)- 6-(6-((4-fluorobenzyl)oxy) pyridin-2-yl)-6-(thiophen- 3-yl)piperidine-2,4-dione was prepared in 24% yield according to Example 2, Step A substituting propan- 2-ol for (4-fluoro- phenyl)methanol | ¹H NMR (400 MHz, DMSO) δ 11.85-11.44 (m, 1H), 8.13 (s, 1H), 7.82-7.73 (m, 1H), 7.49- 7.40 (m, 3H), 7.32-7.22 (m, 3H), 7.20-7.10 (m, 2H), 7.06 (dd, J = 5.1, 1.3 Hz, 1H), 6.93 (t, J = 7.6 Hz, 1H), 6.81 (d, J = 8.2 Hz, 1H), 6.73 (t, J = 7.0 Hz, 1H), 5.93 (d, J = 7.5 Hz, 1H), 5.44 (d, J = 12.4 Hz, 1H), 5.31 (d, J = 12.4 Hz, 1H), 3.82 (d, J = 15.9 Hz, 1H), 3.32 (s, 1H). |
| 300 | SS | 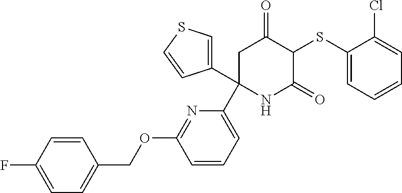 [SMILES: O=C1CC(c2ccsc2)(c2cccc(OCc3ccc(F)cc3)n2)NC(=O)C1Sc1ccccc1Cl] | 3-((2-chlorophenyl)thio)- 6-(6-((4-fluorobenzyl)oxy) pyridin-2-yl)-6-(thiophen- 3-yl)piperidine-2,4-dione was prepared in 22% yield according to Example 2, Step A substituting propan- 2-ol for (4-fluoro- phenyl)methanol | ¹H NMR (400 MHz, DMSO) δ 11.81-11.34 (m, 1H), 8.26 (s, 1H), 7.84-7.75 (m, 1H), 7.51- 7.38 (m, 3H), 7.35-7.21 (m, 3H), 7.14 (dd, J = 12.4, 5.5 Hz, 2H), 7.10-7.01 (m, 1H), 6.94 (t, J = 7.1 Hz, 1H), 6.82 (d, J = 8.1 Hz, 1H), 6.73 (t, J = 7.6 Hz, 1H), 5.92 (d, J = 7.6 Hz, 1H), 5.44 (d, J = 12.4 Hz, 1H), 5.31 (d, J = 12.4 Hz, 1H), 3.86 (d, J = 17.4 Hz, 1H), 3.33 (s, 1H). |
| 301 | SS | 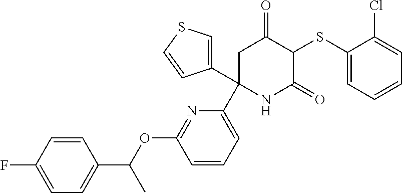 [SMILES: CC(Oc1cccc(C2(c3ccsc3)CC(=O)C(Sc3ccccc3Cl)C(=O)N2)n1)c1ccc(F)cc1] | 3-((2-chlorophenyl)thio)- 6-(6-((1-(4-fluoro- phenyl)ethoxy)pyridin-2- yl)-6-(thiophen-3- yl)piperidine-2,4-dione was prepared in 31% yield according to Example 2, Step A substituting propan- 2-ol for 1-(4-fluoro- phenyl)ethanol | ¹H NMR (400 MHz, DMSO) δ 11.84-11.50 (m, 1H), 8.45-8.20 (m, 1H), 7.74 (dt, J = 6.9, 4.8 Hz, 1H), 7.50-7.43 (m, 2H), 7.35 (ddd, J = 5.6, 4.3, 3.6 Hz, 1H), 7.31- 7.25 (m, 1H), 7.19-7.07 (m, 3H), 6.98-6.80 (m, 2H), 6.78-6.63 (m, 2H), 6.26-6.14 (m, 1H), 5.87 (d, J = 6.7 Hz, 1H), 3.82 (d, J = 16.1 Hz, 1H), 3.35 (d, J = 25.8 Hz, 1H), 1.54 (dd, J = 6.5, 3.3 Hz, 3H), 1.30 (t, J = 4.7 Hz, 1H). |
| 302 | MD | 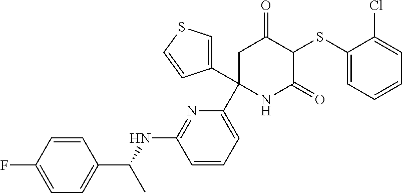 [SMILES: C[C@@H](Nc1cccc(C2(c3ccsc3)CC(=O)C(Sc3ccccc3Cl)C(=O)N2)n1)c1ccc(F)cc1] | 3-((2-chlorophenyl)thio)- 6-(6-((1-(4-fluorophenyl) ethyl)amino)pyridin-2- yl)-6-(thiophen-3-yl) piperidine-2,4-dione was prepared in 7.2% yield according to the Example 4, Step A substituting cyclohexanamine for (R)- 1-(4-fluorophenyl) ethanamine | ¹H NMR (400 MHz, DMSO) δ 11.54-11.37 (m, 1H), 8.23-8.06 (m, 1H), 7.44-7.35 (m, 3H), 7.28- 7.16 (m, 3H), 7.11-7.04 (m, 2H), 6.93 (d, J = 3.9 Hz, 2H), 6.74 (s, 1H), 6.67 (d, J = 6.9 Hz, 1H), 6.48-6.38 (m, 1H), 6.01 (s, 1H), 5.05 (s, 1H), 3.78 (s, 1H), 3.51 (s, 1H), 1.41 (dd, J = 9.4, 7.0 Hz, 3H), 1.26 (d, J = 6.9 Hz, 1H). |
| 303 | MD | 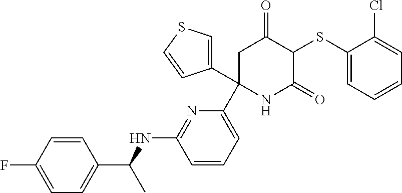 [SMILES: C[C@H](Nc1cccc(C2(c3ccsc3)CC(=O)C(Sc3ccccc3Cl)C(=O)N2)n1)c1ccc(F)cc1] | 3-((2-chlorophenyl)thio)- 6-(6-((1-(4-fluorophenyl) ethyl)amino)pyridin-2- yl)-6-(thiophen-3-yl) piperidine-2,4-dione was prepared in 7.9% yield according to the Example 4, Step A substituting cyclohexanamine for (S)- 1-(4-fluorophenyl) ethanamine | ¹H NMR (400 MHz, DMSO) δ 11.55-11.38 (m, 1H), 8.13 (s, 1H), 7.45-7.35 (m, 3H), 7.30- 7.18 (m, 3H), 7.12-7.03 (m, 2H), 6.99-6.91 (m, 2H), 6.76 (d, J = 11.0 Hz, 1H), 6.68-6.64 (m, 1H), 6.43 (d, J = 25.5 Hz, 1H), 6.00 (d, J = 8.0 Hz, 1H), 5.06 (s, 1H), 3.88-3.78 (m, 1H), 3.51 (s, 1H), 1.41 (dd, J = 9.8, 7.0 Hz, 3H), 1.26 (d, J = 6.9 Hz, 1H). |
| 304 | MD | 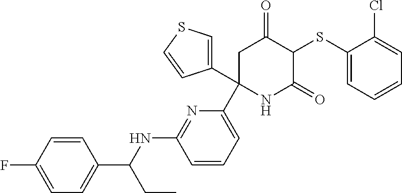 [SMILES: CCC(Nc1cccc(C2(c3ccsc3)CC(=O)C(Sc3ccccc3Cl)C(=O)N2)n1)c1ccc(F)cc1] | 3-((2-chlorophenyl)thio)- 6-(6-((1-(4-fluoro- phenyl)propyl)amino) pyridin-2-yl)-6-(thiophen- 3-yl)piperidine-2,4-dione was prepared in 3.9% yield according to the Example 4, Step A substituting cyclo- hexanamine for 1-(4-fluoro- phenyl)propan-1-amine | m/z: 566.1 89% purity by UV 254 nm |
| 305 | SS | 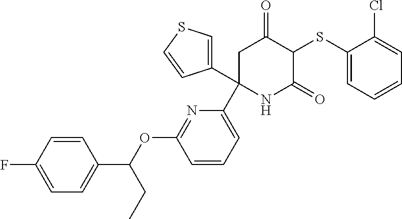 [SMILES: CCC(Oc1cccc(C2(c3ccsc3)CC(=O)C(Sc3ccccc3Cl)C(=O)N2)n1)c1ccc(F)cc1] | 3-((2-chlorophenyl)thio)- 6-(6-((1-(4-fluoro- phenyl)propoxy)pyridin- 2-yl)-6-(thiophen- 3-yl)piperidine-2,4-dione was prepared in 25.8% yield according to Example 2, Step A substituting propan-2-ol for 1-(4-fluoro- phenyl)propan-1-ol | ¹H NMR (400 MHz, DMSO) δ 11.79-11.44 (m, 1H), 8.46-8.18 (m, 1H), 7.76-7.69 (m, 1H), 7.45 (ddd, J = 14.2, 7.8, 5.1 Hz, 2H), 7.34-7.22 (m, 2H), 7.18- 7.01 (m, 3H), 7.00-6.80 (m, 2H), 6.79-6.69 (m, 1H), 6.64 (dd, J = 10.2, 5.1 Hz, 1H), 6.00 (dt, J = 45.5, 6.9 Hz, 1H), 5.86 (dd, J = 15.5, 7.8 Hz, 1H), 3.81 (d, J = 16.3 Hz, 1H), 3.63 (s, 1H), 3.36 (d, J = 20.0 Hz, 1H), 1.95 (dt, J = 14.7, 7.6 Hz, 1H), 1.82 (td, J = 13.7, 6.6 Hz, 1H), 0.95- 0.78 (m, 3H). |
| 306 | MD | 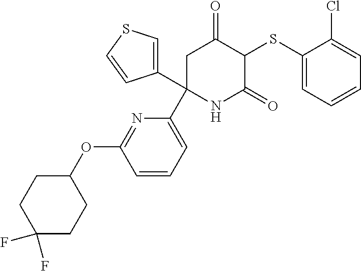 [SMILES: O=C1CC(c2ccsc2)(c2cccc(OC3CCC(F)(F)CC3)n2)NC(=O)C1Sc1ccccc1Cl] | 3-((2-chlorophenyl)thio)- 6-(6-((4,4-difluoro- cyclohexyl)oxy)pyridin- 2-yl)-6-(thiophen- 3-yl)piperidine-2,4-dione was prepared in 22.1% yield according to Example 2, Step A substituting propan-2-ol for 4,4-difluoro- cyclohexanol | ¹H NMR (400 MHz, DMSO) δ 11.55 (s, 1H), 8.39 (s, 1H), 7.84- 7.73 (m, 1H), 7.52 (dd, J = 5.1, 3.0 Hz, 1H), 7.37 (dd, J = 3.0, 1.4 Hz, 1H), 7.31-7.21 (m, 2H), 7.17 (dd, J = 5.1, 1.4 Hz, 1H), 6.95 (td, J = 7.7, 1.5 Hz, 1H), 6.81-6.65 (m, 2H), 5.83 (d, J = 6.7 Hz, 1H), 5.25 (s, 1H), 3.83 (d, J = 16.4 Hz, 1H), 3.34 (d, J = 16.4 Hz, 1H), 2.08-1.66 (m, 8H). |
| 307 | SS | 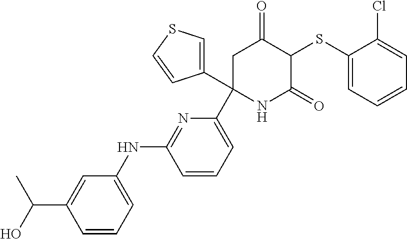 [SMILES: CC(O)c1cccc(Nc2cccc(C3(c4ccsc4)CC(=O)C(Sc4ccccc4Cl)C(=O)N3)n2)c1] | 3-((2-chlorophenyl)thio)- 6-(6-((3-(1-hydroxy- ethyl)phenyl)amino)pyridin- 2-yl)-6-(thiophen-3- yl)piperidine-2,4-dione was prepared in 3.9% yield according to the Example 4, Step A substituting cyclohexanamine for 1- (4-aminophenyl)ethanol | m/z: 550.2 91% purity by UV 254 nm |
| 308 | MD | 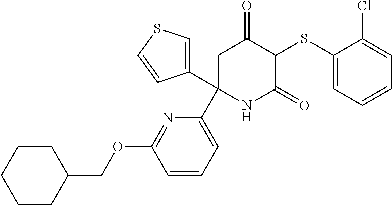 [SMILES: O=C1CC(c2ccsc2)(c2cccc(OCC3CCCCC3)n2)NC(=O)C1Sc1ccccc1Cl] | 3-((2-chlorophenyl)thio)- 6-(6-(cyclohexylmethoxy) pyridin-2-yl)-6-(thiophen- 3-yl)piperidine-2,4-dione was prepared in 36.9% yield according to Example 2, Step A substituting propan-2-ol for cyclo- hexylmethanol | ¹H NMR (400 MHz, DMSO) δ 11.56 (s, 1H), 8.29 (s, 1H), 7.79- 7.70 (m, 1H), 7.50 (dd, J = 5.1, 3.0 Hz, 1H), 7.35 (dd, J = 3.0, 1.4 Hz, 1H), 7.27 (dd, J = 1.9, 1.2 Hz, 1H), 7.24-7.12 (m, 2H), 0.94 (td, J = 7.7, 1.5 Hz, 1H), 6.78-6.68 (m, 2H), 5.89 (d, J = 7.6 Hz, 1H), 4.11 (d, J = 6.4 Hz, 2H), 3.83 (d, J = 17.0 Hz, 1H), 3.32 (d, J = 6.6 Hz, 1H), 1.69 (dd, J = 36.8, 11.2 Hz, 6H), 1.16 (dd, J = 19.2, 10.0 Hz, 3H), 0.98 (s, 2H). |
| 309 | SS | 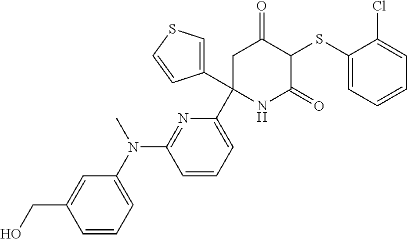 [SMILES: CN(c1cccc(CO)c1)c1cccc(C2(c3ccsc3)CC(=O)C(Sc3ccccc3Cl)C(=O)N2)n1] | 3-((2-chlorophenyl)thio)- 6-(6-((3-(hydroxymethyl) phenyl)(methyl)amino) pyridin-2-yl)-6-(thiophen- 3-yl)piperidine-2,4-dione was prepared in 1.0% yield according to the Example 4, Step A substituting cyclohexanamine for (4- (methylamino)phenyl) methanol | m/z: 550.1 100% purity by UV 254 nm |
| 310 | SS | 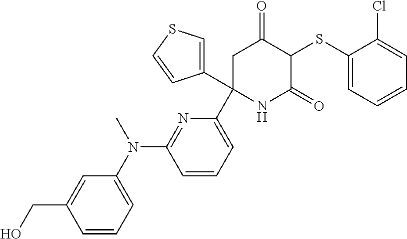 [SMILES: CN(c1cccc(CO)c1)c1cccc(C2(c3ccsc3)CC(=O)C(Sc3ccccc3Cl)C(=O)N2)n1] | 3-((2-chlorophenyl)thio)- 6-(6-((3-(hydroxymethyl) phenyl)(methyl)amino) pyridin-2-yl)-6-(thiophen- 3-yl)piperidine-2,4-dione was prepared in 5.9% yield according to the Example 4, Step A substituting cyclohexanamine for (4- (methylamino)phenyl) methanol | ¹H NMR (400 MHz, DMSO) δ 11.77-11.44 (m, 1H), 8.19-8.04 (m, 1H), 7.54-7.45 (m, 2H), 7.38 (dd, J = 9.2, 6.3 Hz, 2H), 7.28 (d, J = 9.1 Hz, 2H), 7.23-7.12 (m, 3H), 6.95 (t, J = 6.0 Hz, 2H), 6.76 (t, J = 7.6 Hz, 1H), 6.47 (d, J = 8.4 Hz, 1H), 6.02 (d, J = 8.1 Hz, 1H), 5.39-5.18 (m, 1H), 4.50 (s, 2H), 3.81 (s, 2H), 3.42 (s, 3H). |
| 311 | MD | 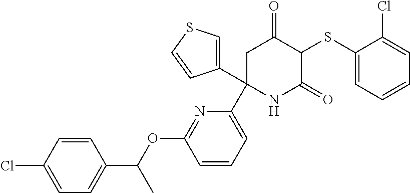 [SMILES: CC(Oc1cccc(C2(c3ccsc3)CC(=O)C(Sc3ccccc3Cl)C(=O)N2)n1)c1ccc(Cl)cc1] | 6-(6-(1-(4-chlorophenyl) ethoxy)pyridin-2-yl)-3- ((2-chlorophenyl)thio)- 6-(thiophen-3-yl)piperidine- 2,4-dione was prepared in 47.6% yield according to Example 2, Step A substituting propan-2-ol for 1-(4-chloro- phenyl)ethanol | ¹H NMR (400 MHz, DMSO) δ 11.61 (s, 1H), 8.30 (d, J = 91.8 Hz, 1H), 7.80-7.71 (m, 1H), 7.49-7.42 (m, 2H), 7.40-7.36 (m, 1H), 7.35- 7.25 (m, 3H), 7.18 (dd, J = 7.1, 4.4 Hz, 1H), 7.07-6.89 (m, 2H), 6.86-6.58 (m, 3H), 6.26-6.10 (m, 1H), 5.85 (d, J = 8.0 Hz, 1H), 3.78 (dd, J = 35.1, 15.3 Hz, 1H), 3.41-3.32 (m, 1H), 1.55 (d, J = 6.4 Hz, 3H). |
| 312 | MD | 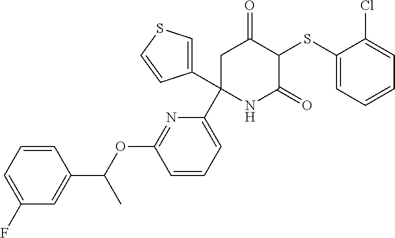 [SMILES: CC(Oc1cccc(C2(c3ccsc3)CC(=O)C(Sc3ccccc3Cl)C(=O)N2)n1)c1cccc(F)c1] | 3-((2-chlorophenyl)thio)- 6-(6-(1-(3-fluorophenyl) ethoxy)pyridin-2-yl)-6- (thiophen-3-yl)piperidine- 2,4-dione was prepared in 20.6% yield according to Example 2, Step A substituting propan-2-ol for 1-(3-fluorophenyl) ethanol | ¹H NMR (400 MHz, DMSO) δ 11.72-11.48 (m, 1H), 8.13 (s, 2H), 7.80-7.72 (m, 1H), 7.48-7.36 (m, 1H), 7.36-7.22 (m, 4H), 7.18 (dd, J = 7.5, 3.5 Hz, 1H), 7.12-7.03 (m, 1H), 6.98-6.88 (m, 1H), 6.82 (dd, J = 21.6, 8.1 Hz, 1H), 6.76-6.47 (m, 2H), 6.19 (dd, J = 35.5, 6.6 Hz, 1H), 5.87 (t, J = 8.0 Hz, 1H), 3.84- 3.63 (m, 1H), 3.14 (s, 1H), 1.61-1.46 (m, 3H).. |
| 313 | MD | 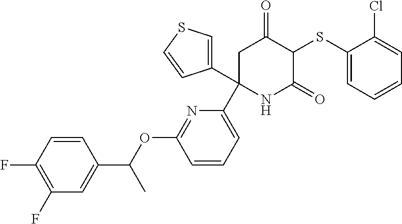 [SMILES: CC(Oc1cccc(C2(c3ccsc3)CC(=O)C(Sc3ccccc3Cl)C(=O)N2)n1)c1ccc(F)c(F)c1] | 3-((2-chlorophenyl)thio)- 6-(6-(1-(3,4-difluoro- phenyl)ethoxy)pyridin-2- yl)-6-(thiophen-3-yl) piperidine-2,4-dione was prepared in 9.6% yield according to Example 2, Step A substituting propan-2-ol for 1-(3,4- difluorophenyl)ethanol | ¹H NMR (400 MHz, DMSO) δ 11.78-11.37 (m, 1H), 8.13 (s, 1H), 7.70 (d, J = 19.1 Hz, 1H), 7.43 (d, J = 11.3 Hz, 1H), 7.09 (dddd, J = 44.9, 29.6, 16.9, 8.5 Hz, 6H), 6.81-6.41 (m, 2H), 5.96 (d, J = 86.0 Hz, 2H), 5.46 (s, 1H), 4.04 (s, 1H), 3.51 (s, 1H), 1.56-1.43 (m, 3H), 1.33-1.23 (m, 1H). |
| 314 | MD | 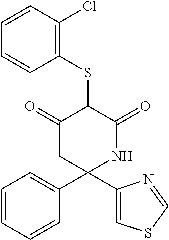 [SMILES: O=C1CC(c2ccccc2)(c2cscn2)NC(=O)C1Sc1ccccc1Cl] | 3-(2-chlorophenyl)sulfanyl- 6-phenyl-6-thiazol-4-yl- piperidine-3,4-dione was prepared according to methods described therein. | ¹H NMR (400 MHz, DMSO-d6) δ 9.15 (d, J = 2.0 Hz, 1H), 8.26 (s, 1H), 7.66 (d, J = 1.9 Hz, 1H), 7.47-7.27 (m, 6H), 7.02-6.89 (m, 1H), 6.84-6.75 (m, 1H), 6.00-5.89 (m, 1H), 3.65 (d, J = 16.5 Hz, 1H), 3.49 (d, J = 16.5 Hz, 1H). |
| 315 | MD | 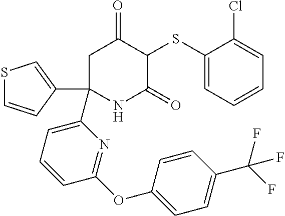 [SMILES: O=C1CC(c2ccsc2)(c2cccc(Oc3ccc(C(F)(F)F)cc3)n2)NC(=O)C1Sc1ccccc1Cl] | 3-(2-chlorophenyl)sulfanyl- 6-(3-thienyl)-6-[6-[4- (trifluoromethyl)phenoxy]- 2-pyridyl]piperidine-2,4- dione was prepared in 41% yield according to the Method 3, Step A substituting 2-chloro-4- fluorophenol for 4- trifluoromethyl)phenol | ¹H NMR (400 MHz, DMSO-d₆) δ = 11.55 (s, 1H), 8.57 (s, 1H), 8.04 (dd, J = 8 Hz, 8 Hz, 1H), 7.75 (d, J = 8.0 Hz, 2H), 7.53 (d, J = 8.0 Hz, 1H), 7.32-7.27 (m, 4H), 7.13 (d, J = 8 Hz, 1H), 7.06 (d, J = 8.0 Hz, 1H), 6.99 (dd, J = 8.0 Hz, 8.0 Hz, 1H), 6.81 (dd, J = 8.0 Hz, 8.0 Hz 1H), 5.93 (d, J = 8.0 Hz, 1H), 3.59 (d, J = 16 Hz, 1H), 3.34 (d, J = 16Hz, 1H) |
| 316 | SS | 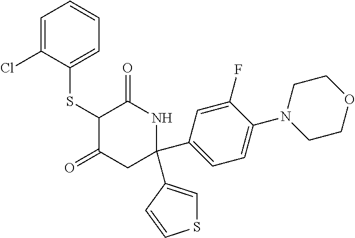 [SMILES: O=C1CC(c2ccsc2)(c2ccc(N3CCOCC3)c(F)c2)NC(=O)C1Sc1ccccc1Cl] | (6S)-3-(2-chlorophenyl) sulfanyl-6-(3-fluoro-4- morpholine-phenyl)-6- (3-thienyl)piperidine-2,4- dione was prepared according to methods described therein | 5.58 min, 517.0, |
| 317 | SS | 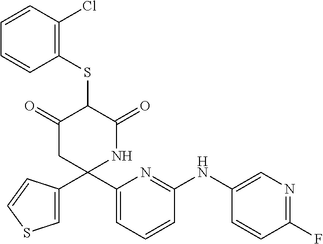 [SMILES: O=C1CC(c2ccsc2)(c2cccc(Nc3ccc(F)nc3)n2)NC(=O)C1Sc1ccccc1Cl] | (6R)-3-(2-chlorophenyl) sulfanyl-6-[6-[(6- fluoro-3-pyridyl)amino]-2- pyridyl]-6-(3-thienyl) piperidine-2,4-dione was prepared as in example 397. | |
| 318 | SS | 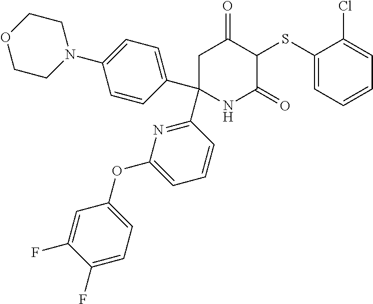 [SMILES: O=C1CC(c2ccc(N3CCOCC3)cc2)(c2cccc(Oc3ccc(F)c(F)c3)n2)NC(=O)C1Sc1ccccc1Cl] | (6S)-3-(2-chlorophenyl) sulfanyl-6-[6-(3,4-difluoro- phenoxy)-2-pyridyl]-6-(4- morpholinophenyl) piperidine-2,4-dione | 1H NMR (400 MHz, METHANOL- d4) d = 7.91 (dd, J = 7.6, 7.6 Hz, 1H), 7.37 (d, J = 7.6, 1H), 7.36-7.21 (m, 4H), 7.04-6.96 (m, 2H), 6.93-6.85 (m, 4H), 6.75 (dd, J = 7.6, 7.6, 1H), 3.84 (dd, J = 4.8, 4.8 Hz, 4H), 3.55 (d, J = 16.8 Hz, 1H), 3.33 (d, J = 16.8 Hz, 1H), 3.16 (dd, J = 4.8, 4.8 Hz, 4H) |
| 319 | SS | 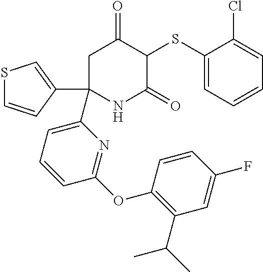 [SMILES: CC(C)c1cc(F)ccc1Oc1cccc(C2(c3ccsc3)CC(=O)C(Sc3ccccc3Cl)C(=O)N2)n1] | (6S)-3-(2-chlorophenyl) sulfanyl-6-[6-(4-fluoro- 2-isopropyl-phenoxy)-2- pyridyl]-6-(3-thienyl) piperidine-2,4-dione was prepared in 17% yield according to method 3. Step A substituting 2- chloro-4-fluorophenol for 4-fluoro-2-isopropylphenol | 1H NMR (400 MHz, METHANOL- d4) d = 7.89 (dd, J = 8.0, 8.0 Hz, 1H), 7.42-7.41 (m, 1H), 7.32 (d, J = 7.2, 1H), 7.25 (d, J = 7.2, 1H), 7.20-7.16 (m, 2H), 6.99- 6.75 (m, 5H), 6.73 (dd, J = 8.0, 8.0 Hz, 1H), 5.87 (d, J = 8.0, 1H), 3.39 (d, J = 16.8 Hz, 1H), 3.11 (d, J = 16.4 Hz, 1H), 2.94- 2.90 (m, 1H), 1.02 (d, J = 6.8 Hz, 3H), 0.98 (d, J = 6.8 Hz, 3H). |
| 320 | SS | 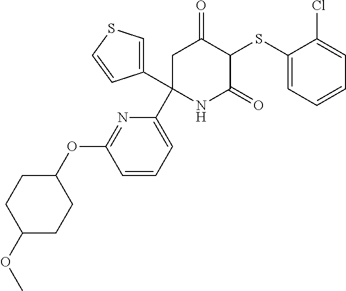 [SMILES: COC1CCC(Oc2cccc(C3(c4ccsc4)CC(=O)C(Sc4ccccc4Cl)C(=O)N3)n2)CC1] | 5-(2-chlorophenyl)sulfanyl- 4-hydroxy-2-[6-(4- methoxycyclohexoxyl)-2- pyridyl]-2-(3-thienyl)- 1,3-dihydropyridin-6-one | |
| 321 | MD | 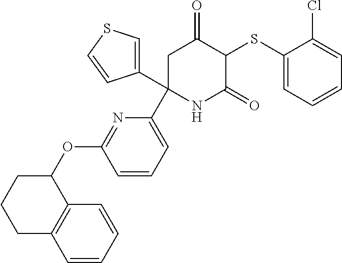 [SMILES: O=C1CC(c2ccsc2)(c2cccc(OC3CCCc4ccccc43)n2)NC(=O)C1Sc1ccccc1Cl] | 5-(2-chlorophenyl)sulfanyl- 4-hydroxy-2-(6-tetralin-1- yloxy-2-pyridyl)-2-(3- thienyl)-1,3-dihydro- pyridin-6-one | |
| 322 | SS | 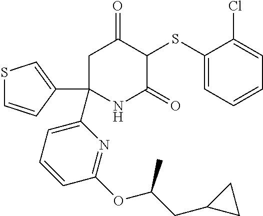 [SMILES: C[C@@H](CC1CC1)Oc1cccc(C2(c3ccsc3)CC(=O)C(Sc3ccccc3Cl)C(=O)N2)n1] | 3-((2-chlorophenyl)thio)- 6-(6-((1-cyclopropyl- propan-2-yl)oxy)pyridin- 2-yl)-6-(thiophen-3-yl) piperidine-2,4-dione was prepared in 15% yield according to the Method 2, Step A substituting propan-2-ol for 1- cyclopropylpropan-2-ol | 1H NMR (400 MHz, METHANOL- d4) d = 7.60 (dd, J = 8.0, 8.0 Hz, 1H), 7.35 (dd, J = 5.6, 1.6 Hz, 1H), 7.17-7.16 (m, 2H), 7.07-7.03 (m, 2H), 6.84 (dd, J = 8.0, 8.0 Hz, 1H), 6.63-6.61 (m, 2H), 5.92 (d, J = 8.0 Hz, 1H), 5.32-5.29 (m, 1H), 3.79 (d, J = 16..0 Hz, 1H), 3.38 (d, J = 16.0 Hz, 1H), 1.60-1.56 (m, 1H), 1.39-1.33 (m, 1H), 1.22 (d, J = 6.0 Hz, 3H), 0.72-0.69 (m, 1H), 0.34-0.31 (m, 2H), 0.06-0.01 (m, 2H) |
| 323 | MD | 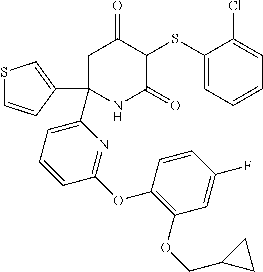 [SMILES: O=C1CC(c2ccsc2)(c2cccc(Oc3ccc(F)cc3OCC3CC3)n2)NC(=O)C1Sc1ccccc1Cl] | 3-(2-chlorophenyl)sulfonyl- 6-[6-[2-(cyclopropyl- methoxy)-4-fluoro- phenoxy]-2-pyridyl]-6-(3- thienyl)piperidine-2,4- dione was prepared in 3.6% yield according to the Method 3, Step A substituting 2-chloro-4- fluorophenol for 2- (cyclopropylmethoxy)- 4-fluorophenol | 1H NMR (400 MHz, METHANOL- d4) δ = 8.56 (s, 1H), 8.05 (dd, J = 8.0, 8.0 Hz, 1H), 7.51 (dd, J = 7.2, 7.2 Hz, 1H), 7.49-7.40 (m, 2H), 7.39 (d, J = 3.2 Hz, 1H), 7.11 (dd, J = 4.8, 4.8 Hz, 1H), 7.07-7.05 (m, 3H), 7.02 (d, J = 6.4 Hz, 1H), 7.01-6.90 (m, 2H), 6.15 (d, J = 8.0 Hz, 1H), 3.87 (dd, J = 3.2, 3.2 Hz, 1H), 3.74 (dd, J = 3.2, 3.2 Hz, 1H), 3.67 (d, J = 16.4 Hz, 1H), 3.37 (d, J = 16.4 Hz, 1H), 0.94-0.90 (m, 1H), 0.39-0.36 (m, 2H), 0.08-0.03 (m, 2H) |
| 324 | MD | 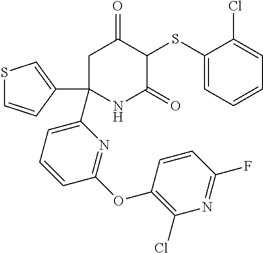 [SMILES: O=C1CC(c2ccsc2)(c2cccc(Oc3ccc(F)nc3Cl)n2)NC(=O)C1Sc1ccccc1Cl] | 6-[6-[(2-chloro-6-fluoro- 3-pyridyl)oxy]-2-pyridyl]- 3-(2-chlorophenyl) sulfanyl-6-(3-thienyl) piperidine-2,4-dione was prepared in 5.6% yield according to the Method 3, Step A substituting 2-chloro-4-fluorophenol for 2-chloro-6-fluoro- pyridin-3-ol | 1H NMR (400 MHz, METHANOL- d4) δ = 7.94 (dd, J = 8.0, 8.0 Hz, 1H), 7.68 (dd, J = 8.0. 8.0 Hz, 1H), 7.37-7.26 (m, 2H), 7.25 (d, J = 3.2 Hz, 1H), 7.23-7.02 (m, 3H), 6.95-6.80 (m, 2H), 6.76 (dd, J = 4.8, 4.2 Hz, 1H), 5.93 (d, J = 7.6 Hz, 1H), 3.41 (d, J = 16.4 Hz, 1H), 3.20 (d, J = 16.4 Hz, 1H) |
| 325 | SS | 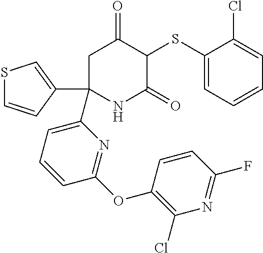 [SMILES: O=C1CC(c2ccsc2)(c2cccc(Oc3ccc(F)nc3Cl)n2)NC(=O)C1Sc1ccccc1Cl] | (6S)-6-[6-[(2-chloro-6- fluoro-3-pyridyl)oxy]-2- pyridyl]-3-(2-chlorophenyl) sulfanyl-6-(3-thienyl) piperidine-2,4-dione was prepared in 5.6% yield according to the Method 3, Step A substituting 2-chloro-4-fluorophenol for 2-chloro-6-fluoro- pyridin-3-o | 1H NMR (400 MHz, METHANOL- d4) δ = 7.94 (dd, J = 8.0, 8.0 Hz, 1H), 7.65 (dd, J = 8.0, 8.0 Hz, 1H), 7.36-7.28 (m, 2H), 7.19 (d, J = 8.0 Hz, 1H), 7.14-7.09 (m, 2H), 7.02 (d, J = 6.4 Hz, 1H), 6.95-6.91 (m, 2H), 6.75 (dd, J = 4.8, 4.8 Hz, 1H), 5.96 (d, J = 7.6 Hz, 1H), 3.39 (d, J = 16.4 Hz, 1H), 3.20 (d, J = 16.4 Hz, 1H) |
| 326 | SS | 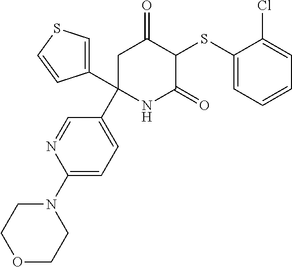 [SMILES: O=C1CC(c2ccc(N3CCOCC3)nc2)(c2ccsc2)NC(=O)C1Sc1ccccc1Cl] | (6S)-6-[6-(4-bromo-2- chloro-phenoxy)-2- pyridyl]-3-(2-chlorophenyl) sulfanyl-6-(3-thienyl) piperidine-2,4-dione was prepared in 1.8% yield according to the Method 3, Step A substituting 2-chloro-4-fluorophenol for 4-bromo-2- chlorophenol | 1H NMR (400 MHz, METHANOL- d4) δ = 7.86 (dd, J = 8.0, 8.0 Hz, 1H), 7.67 (d, J = 2.4 Hz, 1H), 7.49 (dd, J = 8.6, 2.4 Hz, 1H), 7.33-7.16 (m, 2H), 7.15- 7.10 (m, 2H), 7.09 (d, J = 8.8 Hz, 1H), 7.01 (d, J = 8.2 Hz, 1H), 6.95 (d, J = 4.8 Hz, 1H), 6.86 (dd, J = 7.6, 7.6 Hz, 1H), 6.73 (dd, J = 7.2, 7.6 Hz, 1H), 6.05 (d, J = 7.6 Hz, 1H), 3.35 (d, J = 16.4 Hz, 1H), 3.18 (d, J = 16.4 Hz, 1H) |
| 327 | SS | 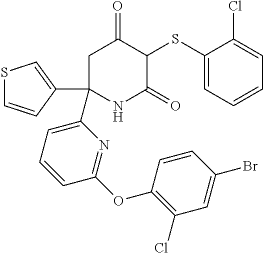 [SMILES: O=C1CC(c2ccsc2)(c2cccc(Oc3ccc(Br)cc3Cl)n2)NC(=O)C1Sc1ccccc1Cl] | (6S)-6-[6-(4-bromo-2- chloro-phenoxy)-2- pyridyl]-3-(2-chlorophenyl) sulfanyl-6-(3-thienyl) piperidine-2,4-dione was prepared in 1.8% yield according to the example 3, Step A substituting 2-chloro-4-fluorophenol for 4-bromo-2- chlorophenol | 1H NMR (400 MHz, METHANOL- d4) δ = 7.86 (dd, J = 8.0, 8.0 Hz, 1H), 7.67 (d, J = 2.4 Hz, 1H), 7.49 (dd, J = 8.6, 2.4 Hz, 1H), 7.33-7.16 (m, 2H), 7.15- 7.10 (m, 2H), 7.09 (d. J = 8.8 Hz, 1H), 7.01 (d, J = 8.2 Hz, 1H), 6.95 (d, J = 4.8 Hz, 1H), 6.86 (dd, J = 7.6, 7.6 Hz, 1H), 6.73 (dd, J = 7.2, 7.6 Hz, 1H), 6.05 (d, J = 7.6 Hz, 1H), 3.35 (d, J = 16.4 Hz, 1H), 3.18 (d, J = 16.4 Hz, 1H) |
| 328 | MD | 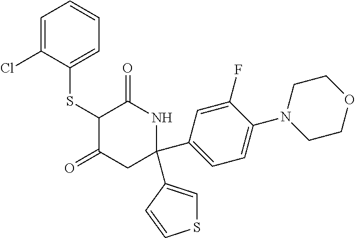 [SMILES: O=C1CC(c2ccsc2)(c2ccc(N3CCOCC3)c(F)c2)NC(=O)C1Sc1ccccc1Cl] | 3-(2-chlorophenyl)sulfanyl- 6-(3-fluoro-4- morpholino-phenyl)-6- (3-thienyl)piperidine- 2,4-dione | ¹H NMR (400 MHz, DMSO-d6) δ 8.44 (s, 1H), 7.58 (dd, J = 5.1, 2.9 Hz, 1H), 7.36-6.96 (m, 7H), 6.79-6.71 (m, 1H), 5.93 (dd, J = 8.0, 1.5 Hz, 1H), 3.74 (dd, J = 5.8, 3.4 Hz, 4H), 3.43 (d, J = 2.3 Hz, 2H), 3.14-2.89 (m, 4H). |
| 329 | MD | 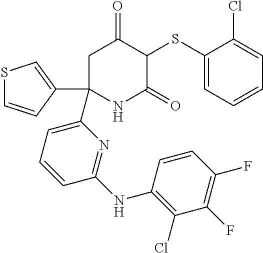 [SMILES: O=C1CC(c2ccsc2)(c2cccc(Nc3ccc(F)c(F)c3Cl)n2)NC(=O)C1Sc1ccccc1Cl] | 6-[6-(2-chloro-3,4-difluoro- anilino)-2-pyridyl]-3-(2- chlorophenyl)sulfanyl-6- (3-thienyl)piperidine-2,4- dione was prepared in 24% yield according to the example 4, Step A substituting cyclo- hexanamine for 2-chloro- 3,4-difluoroaniline. | 1H NMR (400 MHz, DMSO-d6) δ 8.41 (s, 1H), 7.72-7.70 (m, 2H), 7.53 (dd, J = 4.2, 4.2 Hz, 1H), 7.34-7.30 (m, 3H), 7.13-7.12 (m, 2H), 7.01-6.99 (m, 2H), 6.81 (dd, J = 8.0, 8.0 Hz, 1H), 6.00 (d, J = 8.0 Hz 1H), 3.66 (d, J = 16 Hz, 1H), 3.41 (d, J = 16 Hz, 1H) |
| 330 | SS | 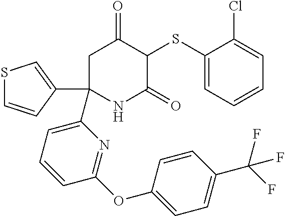 [SMILES: O=C1CC(c2ccsc2)(c2cccc(Oc3ccc(C(F)(F)F)cc3)n2)NC(=O)C1Sc1ccccc1Cl] | (6S)-3-(2-chlorophenyl) sulfanyl-6-(3-thienyl)-6- [6-[4-(trifluoromethyl) phenoxy]-2-pyridyl] piperidine-2,4-dione was prepared in 19% yield according to the example 3, Step A substituting 2- chloro-4-fluorophenol for 4-(trifluoromethyl) phenol. | 1H NMR (400 MHz, DMSO-d6) δ 11.52 (s, 1H), 8.32 (s, 1H), 8.01 (dd, J = 8.0, 8.0 Hz, 1H), 7.74 (d, J = 8.0 Hz, 2H), 7.53-7.49 (m, 2H), 7.28 (m, 4H), 7.11 (d, J = 8.0 Hz, 1H), 7.04 (d, J = 4.8 Hz, 1H), 6.78 (dd, J = 8.0, 8.0 Hz 1H), 6.70 (d, dd, J = 8.0, 8.0 Hz 1H), 5.94 (d, J = 8.0 Hz, 1H), 3.52 (d, J = 16 Hz, 1H), 3.25 (d, J = 16 Hz, 1H) |
| 331 | MD | 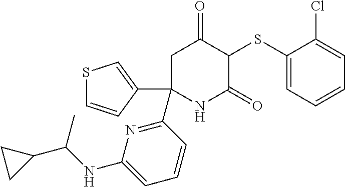 [SMILES: CC(Nc1cccc(C2(c3ccsc3)CC(=O)C(Sc3ccccc3Cl)C(=O)N2)n1)C1CC1] | 3-(2-chlorophenylthio)-6- (6-(1-cyclopropyl- ethylamino)pyridin-2- yl)-6-(thiophen-3-yl) piperidine-2,4-dione was prepared in 2% yield according to the Method 4, Step A substituting cyclohexanamine for 1-cyclopropylethanamine. | 1H NMR (400 MHz, MeOD-d4) δ 7.69 (dd, J = 7.6 Hz, 7.6 Hz, 1H), 7.58 (d, J = 2.8 Hz, 1H), 7.48 (d, J = 2.8 Hz, 1H), 7.25- 7.22 (m, 3H), 6.96 (dd, J = 2.8 Hz, 2.8 Hz, 1H), 6.82 (dd, J = 8.0 Hz, 8.0 Hz, 1H), 6.56 (dd, J = 8.0 Hz, 8.0 Hz, 1H), 6.04 (d, J = 7.2 Hz, 1H), 3.67 (d, J = 16.0 Hz, 1H), 3.46-3.34 (m, 2H), 1.28 (d, J = 2.8 Hz, 3H), 0.99-0.98 (m, 1H), 0.54-0.49 (m, 2H), 0..39-0.30 (m, 2H). |
| 332 | MD | 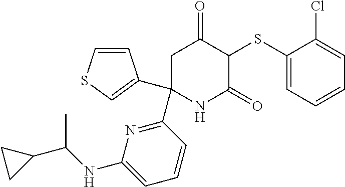 [SMILES: CC(Nc1cccc(C2(c3ccsc3)CC(=O)C(Sc3ccccc3Cl)C(=O)N2)n1)C1CC1] | 3-(2-chlorophenylthio)-6- [6-(2-cyclopropyl- ethylamino)-2-pyridyl]- 6-(3-thienyl) piperidine-2,4-dione was prepared in 2% yield according to the Method 4, Step A substituting cyclohexanamine for 1-cyclopropylethanamine | 1H NMR (400 MHz, MeOD-d4) δ 7.69 (dd, J = 7.6 Hz, 7.6 Hz, 1H), 7.57 (d, J = 2.8 Hz, 1H), 7.47 (d, J = 2.8 Hz, 1H), 7.26- 7.22 (m, 3H), 6.95 (dd, J = 3.2 Hz, 3.2 Hz, 1H), 6.81 (dd, J = 3.2 Hz, 3.2 Hz, 1H), 6.55 (dd, J = 8.0 Hz, 8.0 Hz, 1H), 6.04 (d, J = 7.2 Hz, 1H), 3.64 (d, J = 16.0 Hz, 1H), 3.53-3.43 (m, 2H), 1.29 (d, J = 2.8 Hz, 3H), 1.00-0.98 (m, 1H), 0.54-0.49 (m, 2H), 0.40-0.30 (m, 2H). |
| 333 | MD | 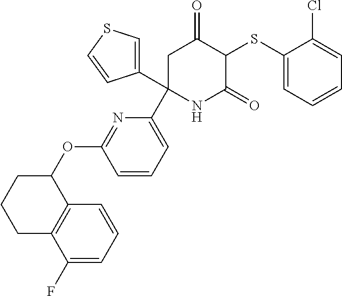 [SMILES: O=C1CC(c2ccsc2)(c2cccc(OC3CCCc4c(F)cccc43)n2)NC(=O)C1Sc1ccccc1Cl] | 5-(2-chlorophenyl)sulfanyl- 2-[6-(5-fluorotetralin-1- yl)oxy-2-pyridyl]-4- hydroxy-2-(3-thienyl)- 1,3-dihydropyridin-6-one | |
| 334 | MD | 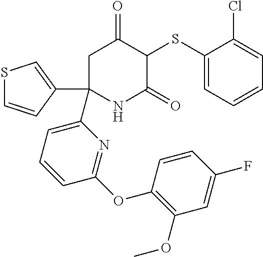 [SMILES: COc1cc(F)ccc1Oc1cccc(C2(c3ccsc3)CC(=O)C(Sc3ccccc3Cl)C(=O)N2)n1] | 3-(2-chlorophenyl)sulfanyl- 6-[6-(4-fluoro-2-methoxy- phenoxy)-2-pyridyl]-6-(3- thienyl)piperidine-2,4- dione was prepared in 38% yield according to the Method 3, Step A substituting 2-chloro-4- fluorophenol for 4- fluoro-2-methoxyphenol. | ¹H NMR (400 MHz, DMSO-d₆) δ = 7.86 (dd, J = 8 Hz, 8 Hz, 1H), 7.49 (dd, J = 4.2, 4.2 Hz, 1H), 7.33-7.27 (m, 2H), 7.20 (d, J = 1.2 Hz, 1H), 6.94 (dd, J = 8.0 Hz, 8.0 Hz, 1H), 6.89 (d, J = 8.0 Hz, 1H), 6.80-6.78 (m, 3H), 6.85-6.74 (m, 2H), 5.95 (d, J = 8.0 Hz, 1H), 4.11 (d, J = 16 Hz, 1H), 3.56, (s, 3H), 3.11 (d, J = 16 Hz, 1H) |
| 335 | SS | 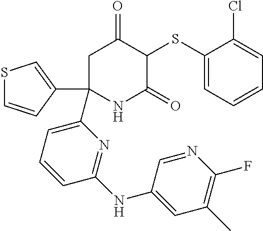 [SMILES: Cc1cc(Nc2cccc(C3(c4ccsc4)CC(=O)C(Sc4ccccc4Cl)C(=O)N3)n2)cnc1F] | (6S)-3-(2-chlorophenyl) sulfanyl-6-[6-[(6-fluoro- 5-methyl-3-pyridyl)amino]- 2-pyridyl]-6-(3-thienyl) piperidine-2,4-dione was prepared in 15% yield according to method 4, Step A substituting cyclohexanamine for 6-fluoro-5-methylpyridin- 3-amine. | 1H NMR (400 MHz, METHANOL- d4) d = 9.28 (s, 1H), 8.16 (s, 1H), 8.09 (d, J = 8.0, 1H), 7.62 (dd, J = 8.0, 8.0 Hz, 1H), 7.47 (dd, J = 4.8, 2.0 Hz, 1H), 7.34 (d, J = 1.6, 1H), 7.23 (d, J = 8.0, 1H), 7.13 (d, J = 5.2, 1H), 7.03 (d, J = 7.6, 1H), 6.90 (dd, J = 8.0, 8.0 Hz, 1H), 6.72 (dd, J = 8.0, 8.0 Hz, 1H), 6.70 (d, J = 7.6, 1H), 6.03 (d, J = 7.6, 1H), 3.62 (d, J = 15.2 Hz, 1H), 3.35 (d, J = 15.2 Hz, 1H), 2.20 (s, 3H) |
| 336 | SS | 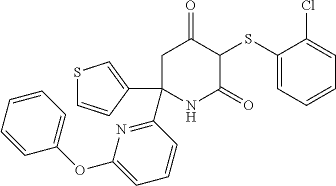 [SMILES: O=C1CC(c2ccsc2)(c2cccc(Oc3ccccc3)n2)NC(=O)C1Sc1ccccc1Cl] | (6R)-3-(2-chlorophenyl) sulfanyl-6-(6-phenoxy- 2-pyridyl)-6-(3-thienyl) piperidine-2,4-dione was prepared in 1.1% yield according to the Method 3, Step A substituting 2- chloro-4-fluorophenol for phenol | 1H NMR (400 MHz, METHANOL- d4) δ = 7.85 (dd, J = 8.0, 8.0 Hz, 1H), 7.38-7.36 (m, 2H), 7.34 (d, J = 4.8 Hz, 1H), 7.30 (d, J = 7.2 Hz, 1H), 7.18-7.15 (m, 2H), 7.14 (d, J = 3.2 Hz, 1H), 7.06- 7.04 (m, 2H), 7.03 (d, J = 4.8 Hz, 1H), 6.95 (m, 2H), 6.76 (dd, J = 8.0, 8.0 Hz, 1H), 6.01 (d, J = 8.0, 8.0 Hz, 1H), 3.64 (d, J = 16.4 Hz, 1H), 3.32 (d, J = 16.4 Hz, 1H) |
| 337 | SS | 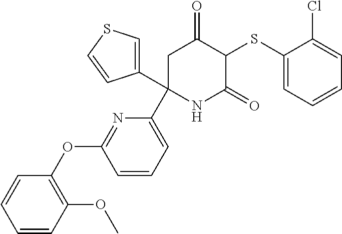 [SMILES: COc1ccccc1Oc1cccc(C2(c3ccsc3)CC(=O)C(Sc3ccccc3Cl)C(=O)N2)n1] | 5-(2-chlorophenyl)sulfanyl- 4-hydroxy-2-[6-(2- methoxyphenoxy)-2- pyridyl]-2-(3-thienyl)- 1,3-dihydropyridin-6-one | |
| 338 | SS | 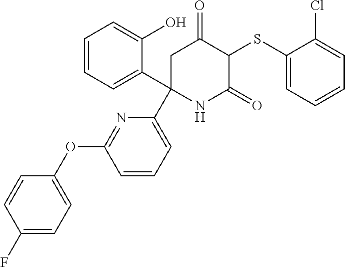 [SMILES: O=C1CC(c2cccc(Oc3ccc(F)cc3)n2)(c2ccccc2O)NC(=O)C1Sc1ccccc1Cl] | 5-(2-chlorophenyl)sulfanyl- 2-[6-(4-fluorophenoxy)- 2-pyridyl]-4-hydroxy-2- (2-hydroxyphenyl)-1,3- dihydropyridin-6-one | |
| 339 | MD | 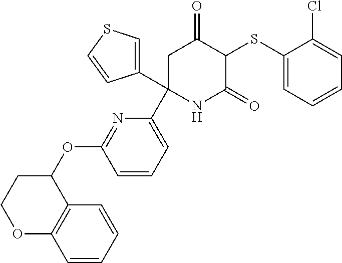 [SMILES: O=C1CC(c2ccsc2)(c2cccc(OC3CCOc4ccccc43)n2)NC(=O)C1Sc1ccccc1Cl] | 5-(2-chlorophenyl)sulfanyl- 2-(6-chroman-4-yloxy- 2-pyridyl)-4-hydroxy-2- (3-thienyl)-1,3-dihydro- pyridin-6-one | |
| 340 | MD | 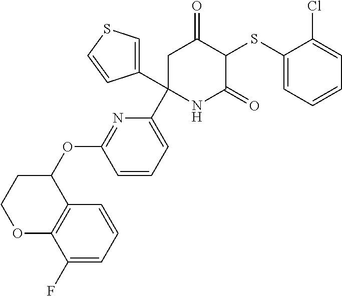 [SMILES: O=C1CC(c2ccsc2)(c2cccc(OC3CCOc4c(F)cccc43)n2)NC(=O)C1Sc1ccccc1Cl] | 5-(2-chlorophenyl)sulfanyl- 2-[6-(8-fluorochroman- 4-yl)oxy-2-pyridyl]-4- hydroxy-2-(3-thienyl)- 1,3-dihydropyridin-6-one | |
| 341 | SS | 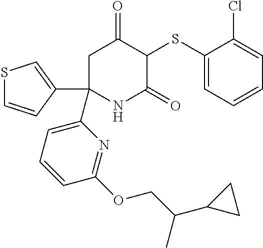 [SMILES: CC(COc1cccc(C2(c3ccsc3)CC(=O)C(Sc3ccccc3Cl)C(=O)N2)n1)C1CC1] | 3-((2-chlorophenyl)thio)- 6-(6-(2-cyclopropyl- propoxy)pyridin-2-yl)- 6-(thiophen-3-yl)piperidine- 2,4-dione was prepared in 15% yield according to the Method 2, Step A substituting propan-2- ol for 2-cyclopropyl- propan-1-ol | 1H NMR (400 MHz, METHANOL- d4) d = 7.70 (dd, J = 8.0, 8.0 Hz, 1H), 7.42 (dd, J = 4.0, 1.6 Hz, 1H), 7.26-7.11 (m, 4H), 6.92 (dd, J = 8.0, 8.0 Hz, 1H), 6.76- 6.74 (m, 2H), 5.99 (d, J = 8 Hz, 1H), 4.43-4.38 (m, 1H), 4.22- 4.16 (m, 1H), 3.85 (d, J = 16 Hz, 1H), 3.47 (d, J = 16 Hz, 1H), 1.15-1.03 (m, 4H), 0.62-0.60 (m, 1H), 0.39-0.37 (m, 2H), 0.15-0.03 (m, 2H) |
| 342 | MD | 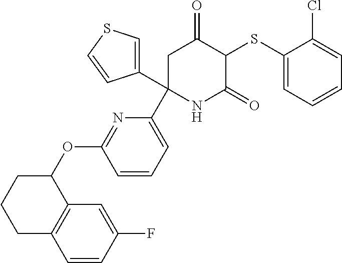 [SMILES: O=C1CC(c2ccsc2)(c2cccc(OC3CCCc4ccc(F)cc43)n2)NC(=O)C1Sc1ccccc1Cl] | 5-(2-chlorophenyl)sulfanyl- 2-[6-(7-fluorotetralin- 1-yl)oxy-2-pyridyl]-4- hydroxy-2-(3-thienyl)- 1,3-dihydropyridin-6- one was prepared according to methods described herein. | |
| 343 | MD | 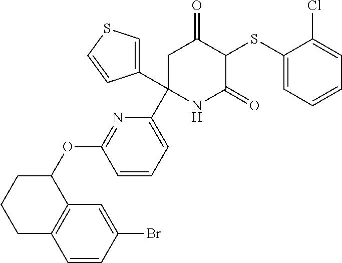 [SMILES: O=C1CC(c2ccsc2)(c2cccc(OC3CCCc4ccc(Br)cc43)n2)NC(=O)C1Sc1ccccc1Cl] | 2-[6-(7-bromotetralin-1- yl)oxy-2-pyridyl]-5-(2- chlorophenyl)sulfanyl-4- hydroxy-2-(3-thienyl)-1,3- dihydropyridin-6-one was prepared according to methods described herein. | |
| 344 | MD | 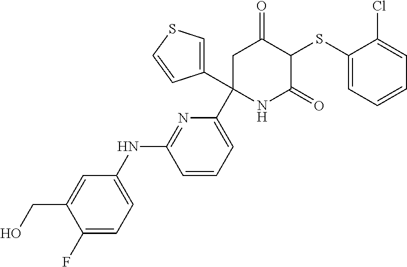 [SMILES: O=C1CC(c2ccsc2)(c2cccc(Nc3ccc(F)c(CO)c3)n2)NC(=O)C1Sc1ccccc1Cl] | 5-(2-chlorophenyl)sulfanyl- 2-[6-[4-fluoro-3- (hydroxymethyl)anilino]- 2-pyridyl]-4-hydroxy-2- (3-thienyl)-1,3-dihydro- pyridin-6-one | |
| 345 | MD | 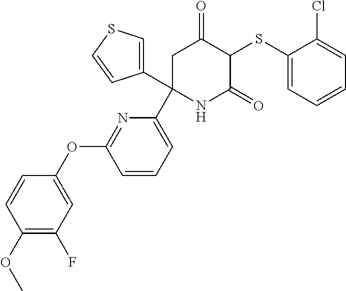 [SMILES: COc1ccc(Oc2cccc(C3(c4ccsc4)CC(=O)C(Sc4ccccc4Cl)C(=O)N3)n2)cc1F] | 5-(2-chlorophenyl)sulfanyl- 2-[6-(3-fluoro-4- methoxy-phenoxy)-2- pyridyl]-4-hydroxy-2- (3-thienyl)-1,3-dihydro- pyridin-6-one | |
| 346 | SS | 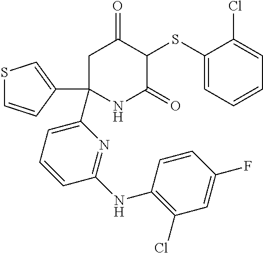 [SMILES: O=C1CC(c2ccsc2)(c2cccc(Nc3ccc(F)cc3Cl)n2)NC(=O)C1Sc1ccccc1Cl] | (6S)-6-[6-(2-chloro-4- fluoro-anilino)-2-pyridyl]- 3-(2-chlorophenyl)sulfanyl- 6-(3-thienyl)piperidine-2,4- dione was prepared in 12.4% yield according to the Method 4, Step A substituting cyclo- hexanamine for 2-chloro- 4-fluorobenzenamine | 1H NMR (400 MHz, METHANOL- d4) δ = 7.86 (dd, J = 5.2, 5.2 Hz, 1H), 7.58 (dd, J = 6.4, 6.4 Hz, 1H), 7.28 (dd, J = 5.4, 3.2 Hz, 1H), 7.22 (s, 1H), 7.21-7.13 (m, 2H), 7.12 (d, J = 5.2 Hz, 1H), 7.08-7.06 (m, 2H), 7.04 (dd, J = 6.4, 6.4 Hz, 1H), 6.85 (dd, J = 5.2, 5.2 Hz, 1H), 6.82-6.81 (m, 2H), 6.09 (d, J = 7.2 Hz, 1H), 3.76 (d, J = 16.4 Hz, 1H), 3.44 (d, J = 16.4 Hz, 1H) |
| 347 | SS | 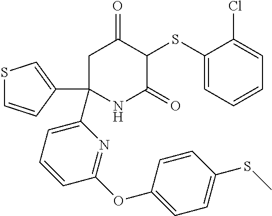 [SMILES: CSc1ccc(Oc2cccc(C3(c4ccsc4)CC(=O)C(Sc4ccccc4Cl)C(=O)N3)n2)cc1] | (6S)-3-(2-chlorophenyl) sulfanyl-6-[6-(4-methyl- sulfanylphenoxy)-2-pyridyl]- 6-(3-thienyl)piperidine-2,4- dione was prepared in 18% yield according to method 3, Step A substituting 2-chloro-4- fluorophenol for 4- (methylthio)phenol. (6S)-3-(2-chlorophenyl) sulfanyl-6-[6-(4-methyl- sulfanylphenoxy)-2-pyridyl]- 6-(3-thienyl)piperidine-2,4- | 1H NMR (400 MHz, METHANOL- d4) d = 7.87 (dd, J = 8.4, 8.4 Hz, 1H), 7.38 (dd, J = 4.0, 4.0 Hz, 1H), 7.33-7.28 (m, 3H), 7.23 (d, J = 8.4 Hz, 1H), 7.18 (d, J = 4.0 Hz, 1H), 7.03-6.95 (m, 5H), 6.76 (dd, J = 8.4, 8.4 Hz, 1H), 6.02 (dd, J = 8.0, 1.6 Hz, 1H), 3.75 (d, J = 16.4 Hz, 1H), 3.32 (d, J = 16.4 Hz, 1H), 2.47 (s, 3H) |
| | | | dione was prepared in | |
| | | | 18% yield according to | |
| | | | method 3, Step A | |
| | | | substituting 2-chloro-4- | |
| | | | fluorophenol for 4- | |
| | | | (methylthio)phenol. | |
| 348 | SS | 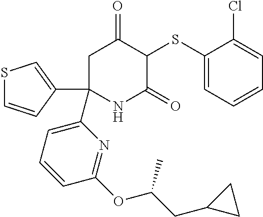 [SMILES: C[C@H](CC1CC1)Oc1cccc(C2(c3ccsc3)CC(=O)C(Sc3ccccc3Cl)C(=O)N2)n1] | 3-((2-chlorophenyl)thio)- 6-(6-((1-cyclopropyl- propan-2-yl)oxy)pyridin- 2-yl)-6-(thiophen-3-yl) piperidine-2,4-dione was prepared in 15% yield according to the Method 2, Step A substituting propan-2-ol for 1- cyclopropylpropan-2-ol | 1H NMR (400 MHz, METHANOL- d4) d = 7.69 (dd, J = 8.0, 8.0 Hz, 1H), 7.41 (dd, J = 7.6, 2.8 Hz, 1H), 7.26-7.18 (m, 2H), 7.14-7.09 (m, 2H), 6.72 (dd, J = 8.0, 8.0 Hz, 1H), 6.70- 6.68 (m, 2H), 5.98 (d, J = 8.0 Hz, 1H), 5.39-5.34 (m, 1H), 3.85 (d, J = 16..0 Hz, 1H), 3.42 (d, J = 16.0 Hz, 1H), 1.63-1.59 (m, 1H), 1.43-1.33 (m, 4H), 0.70-0.67 (m, 1H), 0.31-0.26 (m, 2H), 0.04-0.01 (m, 2H) |
| 349 | SS | 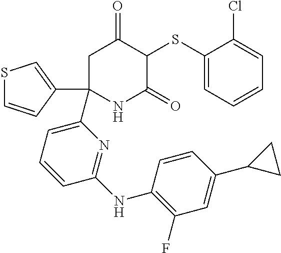 [SMILES: O=C1CC(c2ccsc2)(c2cccc(Nc3ccc(C4CC4)cc3F)n2)NC(=O)C1Sc1ccccc1Cl] | (6R)-3-(2-chlorophenyl) sulfanyl-6-[6-(4-cyclo- propyl-2-fluoro-anilino)- 2-pyridyl]-6-(3-thienyl) piperidine-2,4-dione was prepared in 6% yield according to the Method 4, Step A substituting cyclohexanamine for 4- cyclopropyl-2-fluoroaniline. | 1H NMR (400 MHz, MeOD-d4) δ 7.68-7.65 (m, 2H), 7.49 (d, J = 3.2 Hz, 1H), 7.38 (d, J = 1.2 Hz, 1H), 7.23-7.21 (m, 2H), 6.90 (d, J = 3.2 Hz, 1H), 6.89-6.81 (m, 5H), 6.09 (dd, J = 6.8 Hz, 1.2 Hz, 1H), 3.76 (d, J = 16.0 Hz, 1H), 3.51 (d, J = 16.0 Hz, 1H), 1.91-1.88 (m, 1H), 0.99- 0.95 (m, 2H), 0.69-0.66 (m, 2H). |
| 350 | MD | 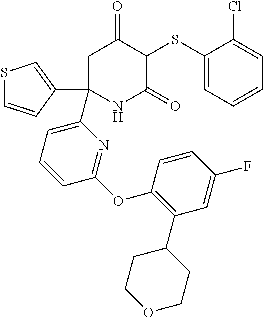 [SMILES: O=C1CC(c2ccsc2)(c2cccc(Oc3ccc(F)cc3C3CCOCC3)n2)NC(=O)C1Sc1ccccc1Cl] | 3-(2-chlorophenyl)sulfanyl- 6-[6-(4-fluoro-2-tetrahydro- pyran-4-yl-phenoxy)-2- pyridyl]-6-(3-thienyl) piperidine-2,4-dione was prepared in 5% yield according to method 3. Step A substituting 2- chloro-4-fluorophenol for 4-fluoro-2-(tetrahydro-2H- pyran-4-yl)phenol. | 1H NMR (400 MHz, METHANOL- d4) d = 11.36 (s, 1H), 8.46 (s, 1H), 7.92 (dd, J = 8.0, 8.0, 1H), 7.45 (dd, J = 8.0, 1.2 Hz, 1H), 7.37 (d, J = 7.6 Hz, 1H), 7.27 (d, J = 7.6, 1H), 7.20 (d, J = 1.2, 1H), 7.19 (d, J = 1.2, 1H), 7.05- 7.03 (m, 2H), 7.00-6.94 (m, 3H), 6.72 (dd, J = 8.0, 8.0 Hz, 1H), 5.79 (d, J = 7.6, 1H), 3.66-3.61 (m, 2H), 3.49 (d, J = 16.4 Hz, 1H), 3.12 (d, J = 16.4 Hz, 1H), 3.02 (dd, J = 8.0, 8.0 Hz, 1H), 2.94 (dd, J = 8.0, 8.0 Hz, 1H), 2.76 (dd, J = 8.0, 8.0 Hz, 1H), 1.58-1.37 (m, 4H). |
| 351 | SS | 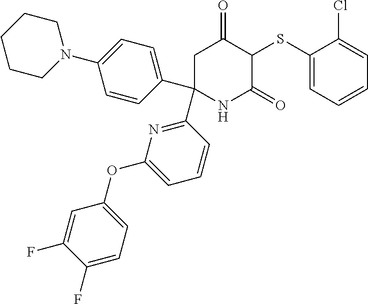 [SMILES: O=C1CC(c2ccc(N3CCCCC3)cc2)(c2cccc(Oc3ccc(F)c(F)c3)n2)NC(=O)C1Sc1ccccc1Cl] | (6R)-3-(2-chlorophenyl) sulfanyl-6-[6-(3,4-difluoro- phenoxy)-2-pyridyl]-6-[4- (1-piperidyl)phenyl] piperidine-2,4-dione was prepared in 11% yield according to the Method 40, Step A substituting morpholine for piperidine | ¹H NMR (400 MHz, DMSO-d₆) δ = 11.42, (s, 1H), 8.31 (s, 1H), 7.95 (dd, J = 8 Hz, 8 Hz, 1H), 7.45-7.29 (m, 2H), 7.11-6.97 (m, 5H), 6.85 (d, J = 8 Hz, 1H), 6.73 (dd, J = 8.0 8.0 Hz, 1H), 5.85 (d, J = 8.0 Hz, 1H), 3.46 (d, J = 16 Hz, 1H), 3.20 (d, J = 16 Hz, 1H), 3.13-3.10 (m, 4H), 1.59-1.52 (m, 6H) |
| 352 | MD | 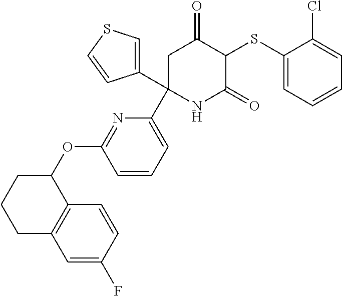 [SMILES: O=C1CC(c2ccsc2)(c2cccc(OC3CCCc4cc(F)ccc43)n2)NC(=O)C1Sc1ccccc1Cl] | 5-(2-chlorophenyl)sulfanyl- 2-[6-(6-fluorotetralin-1- yl)oxy-2-pyridyl]-4- hydroxy-2-(3-thienyl)- 1,3-dihydropyridin-6-one | |
| 353 | SS | 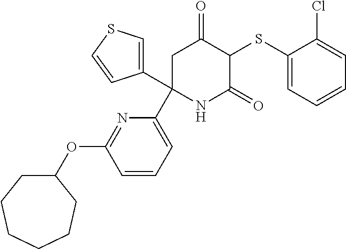 [SMILES: O=C1CC(c2ccsc2)(c2cccc(OC3CCCCCC3)n2)NC(=O)C1Sc1ccccc1Cl] | 5-(2-chlorophenyl)sulfanyl- 2-[6-(cycloheptoxy)-2- pyridyl]-4-hydroxy-2- (3-thienyl)-1,3-dihydro- pyridin-6-one | |
| 354 | SS | 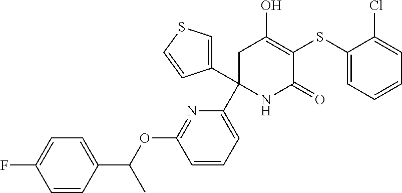 [SMILES: CC(Oc1cccc(C2(c3ccsc3)CC(O)=C(Sc3ccccc3Cl)C(=O)N2)n1)c1ccc(F)cc1] | 5-(2-chlorophenyl)sulfanyl- 2-[6-[1-(4-fluorophenyl) ethoxy]-2-pyridyl]-4- hydroxy-2-(3-thienyl)- 1,3-dihydropyridin-6-one | |
| 355 | MD | 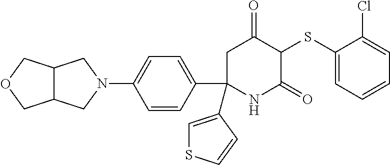 [SMILES: O=C1CC(c2ccc(N3CC4COCC4C3)cc2)(c2ccsc2)NC(=O)C1Sc1ccccc1Cl] | 6-[4-(1,3,3a,4,6,6a-hexa- hydrofuro[3,4-c]pyrrol-5- yl)phenyl]-3-(2-chloro- phenyl)sulfanyl-6-(3- thienyl)piperidine-2,4- dione was prepared in 4% yield according to the Method 7, Step H substituting 2-methyl- morpholine for hexahydro- 1H-furo[3,4-c]pyrrole. | 1H NMR (400 MHz, MeOD-d4) δ 7.47 (dd, J = 5.2 Hz, 5.2 Hz, 1H), 7.28 (d, J = 8.8 Hz, 2H), 7.23 (d, J = 3.2 Hz, 1H), 7.21 (d, J = 3.2 Hz, 1H), 7.20 (d, J = 3.2 Hz, 1H), 7.13 (d, J = 3.2 Hz, 1H), 6.76 (d, J = 8.8 Hz, 2H), 6.71 (dd, J = 7.6 Hz, 7.6 Hz, 1H), 5.93 (d, J = 7.6 Hz, 1H), 3.93 (dd, J = 2.0 Hz, 2.0 Hz, 1H), 3.67 (dd, J = 2.0 Hz, 2.0 Hz, 1H), 3.47-3.42 (m, 4H), 3.27 (d, J = 2.0 Hz, 2H), 3.09 (d, J = 2.0 Hz, 2H). |
| 356 | MD | 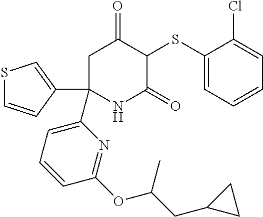 [SMILES: CC(CC1CC1)Oc1cccc(C2(c3ccsc3)CC(=O)C(Sc3ccccc3Cl)C(=O)N2)n1] | 3-((2-chlorophenyl)thio)- 6-[6-((1-cyclopropyl- propan-2-yl)oxy)pyridin- 2-yl)-6-(thiophen-3-yl) piperidine-2,4-dione was prepared in 33% yield according to the Method 2, Step A substituting propan-2-ol for 1-cyclo- propylpropan-2-ol | 1H NMR (400 MHz, METHANOL- d4) d = 7.69 (dd, J = 8, 4 Hz, 1H), 7.42 (dd, J = 4.4, 2.8 Hz, 1H), 7.26-7.22 (m, 2H), 7.14-7.10 (m, 2H), 6.91 (dd, J = 4.0, 2.0 Hz, 1H), 6.75-6.73 (m, 2H), 5.98 (dd, J = 9.2, 1.6 Hz, 1H), 5.41-5.34 (m, 1H), 3.89 (d, J = 16.4 Hz, 1H), 3.45 (d, J = 16.4 Hz, 1H), 1.65- 1.60 (m, 1H), 1.43-1.28 (m, 4H), 0.73-0.69 (m, 1H), 0.40-0.38 (m, 2H), 0.07-0.00 (m, 2H) |
| 357 | MD | 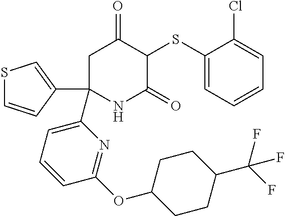 [SMILES: O=C1CC(c2ccsc2)(c2cccc(OC3CCC(C(F)(F)F)CC3)n2)NC(=O)C1Sc1ccccc1Cl] | 3-(2-chlorophenyl)sulfanyl- 6-(3-thienyl)-6-[6-[4- (trifluoromethyl)cyclo- hexoxy]-2-pyridyl] piperidine-2,4-dione was prepared in 23% yield according to the Method 2, Step A substituting propan-2-ol for 4- (trifluoromethyl)cyclo- hexanol | 1H NMR (400 MHz, METHANOL- d4) d = 7.71 (dd, J = 8, 4 Hz, 1H), 7.42 (dd, J = 4.4, 2.8 Hz, 1H), 7.24-7.20 (m, 2H), 7.16-7.11 (m, 2H), 6.93 (dd, J = 8.0, 8.0 Hz, 1H), 6.71-6.69 (m, 2H), 5.92 (dd, J = 8.0, 4.0 Hz, 1H), 5.03- 4.86 (m, 1H), 3.85 (d, J = 16.0 Hz, 1H), 3.44 (d, J = 16.0 Hz, 1H), H), 2.21-1.96 (m, 4H), 1.60- 1.34 (m, 5H) |
| 358 | SS | 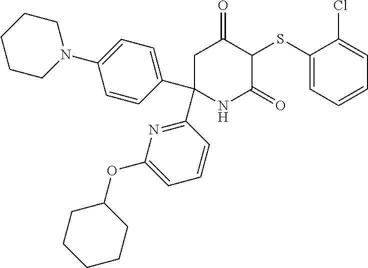 [SMILES: O=C1CC(c2ccc(N3CCCCC3)cc2)(c2cccc(OC3CCCCC3)n2)NC(=O)C1Sc1ccccc1Cl] | (6S)-3-(2-chlorophenyl) sulfanyl-6-[6-(cyclo- hexoxy)-2-pyridyl]-6- [4-(1-piperidyl)phenyl] piperidine-2,4-dione was prepared in 8% yield according to the Method 41, Step A substituting morpholine for piperidine | ¹H NMR (400 MHz, DMSO-d₆) δ = 7.70 (dd, J = 8 Hz, 8 Hz, 1H), 7.25 (d, J = 7.2 Hz, 1H), 7.19-7.16 (m, 2H), 6.92-6.85 (m, 3H), 6.80 (d, J = 8.0 Hz, 1H), 6.68-6.64 (m, 2H), 5.84 (d, J = 8.0 Hz, 1H), 5.01-4.99 (m, 1H), 3.39 (d, J = 16 Hz, 1H), 3.29 (d, J = 16 Hz, 1H), 3.11-3.05 (m, 4H), 1.91-1.84 (m, 2H), 1.69-1.51 (m, 9H), 1.40- 1.23 (m, 5H) |
| 359 | MD | 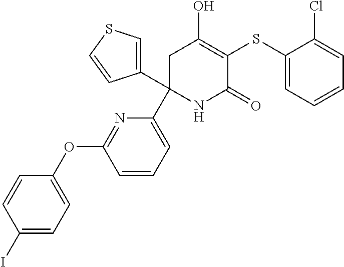 [SMILES: O=C1NC(c2ccsc2)(c2cccc(Oc3ccc(I)cc3)n2)CC(O)=C1Sc1ccccc1Cl] | 5-(2-chlorophenyl)sulfanyl- 4-hydroxy-2-[6-(4- iodophenoxy)-2-pyridyl]- 2-(3-thienyl)-1,3-dihydro- pyridin-6-one | |
| 360 | SS | 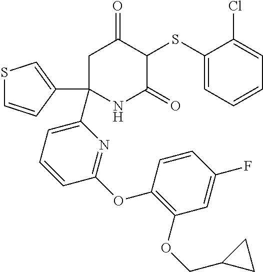 [SMILES: O=C1CC(c2ccsc2)(c2cccc(Oc3ccc(F)cc3OCC3CC3)n2)NC(=O)C1Sc1ccccc1Cl] | (6S)-3-(2-chlorophenyl) sulfanyl-6-[6-[2-(cyclo- propylmethoxy)-4-fluoro- phenoxy]-2-pyridyl]-6- (3-thienyl)piperidine-2,4- dione was prepared in 16% yield according to the Method 3, Step A substituting 2-chloro-4- fluorophenol for 2- (cyclopropylmethoxy)- 4-fluorophenol | ¹H NMR (400 MHz, DMSO-d₆) δ = 7.83 (dd, J = 8 Hz, 8 Hz, 1H), 7.42 (dd, J = 4.2 4.2 Hz 1H), 7.32 (d, J = 7.2 Hz, 1H), 7.19- 7.15 (m, 3H), 7.03 (d, J = 8.0 Hz, 1H), 6.90-6.80 (m, 5H), 6.07 (d, J = 8.0 Hz, 1H), 3.69 (dd, J = 6.8 Hz, 6.8 Hz, 1H), 3.60 (dd, J = 6.8 Hz, 6.8 Hz, 1H), 3.42 (d, J = 16 Hz, 1H), 3.04 (d, J = 16 Hz, 1H), 0.81-0.77 (m, 1H), 0.23- 0.21 (m, 2H), 0.12-0.10 (m, 2H) |
| 361 | SS | 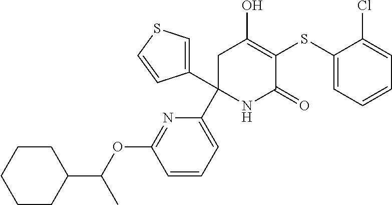 [SMILES: CC(Oc1cccc(C2(c3ccsc3)CC(O)=C(Sc3ccccc3Cl)C(=O)N2)n1)C1CCCCC1] | 5-(2-chlorophenyl)sulfanyl- 2-[6-(1-cyclohexyl- ethoxy)-2-pyridyl]-4- hydroxy-2-(3-thienyl)-1,3- dihydropyridin-6-one | |
| 362 | MD | 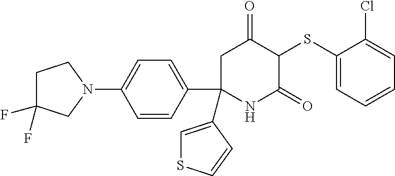 [SMILES: O=C1CC(c2ccc(N3CCC(F)(F)C3)cc2)(c2ccsc2)NC(=O)C1Sc1ccccc1Cl] | 3-(2-chlorophenyl)sulfanyl- 6-[4-(3,3-difluoro- pyrrolidin-1-yl)phenyl]- 6-(3-thienyl)piperidine- 2,4-dione was prepared in 18% yield according to the Method 4, Step A substituting cyclo- hexanamine for 3,3- difluoropyrrolidine | ¹H NMR (400 MHz, DMSO-d₆) δ = 7.49 (dd, J = 8.0, 8.0 Hz, 1H), 7.31-7.26 (m, 3H), 7.20- 7.15 (m, 2H), 6.88 (dd, J = 8.0, 8.0 Hz, 1H), 6.66-6.64 (m, 3H), 5.98 (d, J = 7.6 Hz, 1H), 3.68 (t, J = 13.2 Hz, 2H), 3.54 (t, J = 7.2 Hz, 2H), 3.32 (s, 2H), 2.58-2.47 (m, 2H) |
| 363 | MD | 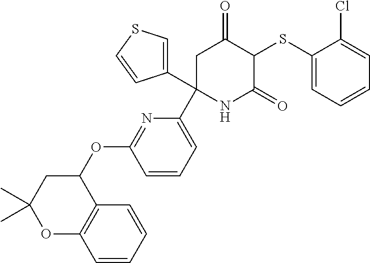 [SMILES: CC1(C)CC(Oc2cccc(C3(c4ccsc4)CC(=O)C(Sc4ccccc4Cl)C(=O)N3)n2)c2ccccc2O1] | 5-(2-chlorophenyl)sulfanyl- 2-[6-(2,2-dimethyl- chroman-4-yl)oxy-2- pyridyl]-4-hydroxy-2- (3-thienyl)-1,3-dihydro- pyridin-6-one | |
| 364 | SS | 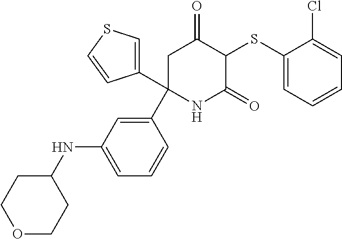 [SMILES: O=C1CC(c2ccsc2)(c2cccc(NC3CCOCC3)c2)NC(=O)C1Sc1ccccc1Cl] | (6S)-3-(2-chlorophenyl) sulfanyl-6-[3-(tetrahydro- pyran-4-ylamino)phenyl]- 6-(3-thienyl)piperidine- 2,4-dione was prepared in 10% yield according to the example 7, Step H substituting 2-methyl- morpholine for tetrahydro-2H-pyran- 4-amine. | 1H NMR (400 MHz, MeOD-d4) δ 7.53 (dd, J = 5.2 Hz, 5.2 Hz, 1H), 7.32 (dd, J = 5.2 Hz, 5.2 Hz, 2H), 7.21 (d, J = 3.2 Hz, 1H), 7.18-7.17 (m, 4H), 6.94 (dd, J = 3.2 Hz, 3.2 Hz, 1H), 6.76 (dd, J = 4.8 Hz, 4.8 Hz, 1H), 5.95 (dd, J = 6.8 Hz, 6.8 Hz, 1H), 3.94 (d, J = 5.6 Hz, 2H), 3.59-3.56 (m, 1H), 3.51 (d, J = 5.6 Hz, 2H), 3.37 (d, J = 16.0 Hz, 2H), 1.86- 1.83 (m, 2H), 1.59-1.56 (m, 2H). |
| 365 | SS | 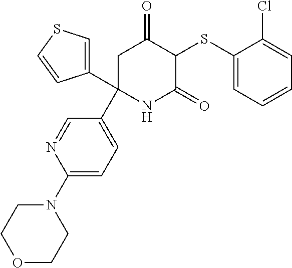 [SMILES: O=C1CC(c2ccc(N3CCOCC3)nc2)(c2ccsc2)NC(=O)C1Sc1ccccc1Cl] | 5-(2-chlorophenyl)sulfanyl- 4-hydroxy-2-(6- morpholine-3-pyridyl)-2- (3-thienyl)-1,3-dihydro- pyridin-6-one was prepared according to methods described herein. | |
| 366 | SS | 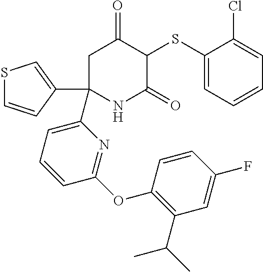 [SMILES: CC(C)c1cc(F)ccc1Oc1cccc(C2(c3ccsc3)CC(=O)C(Sc3ccccc3Cl)C(=O)N2)n1] | (6R)-3-(2-chlorophenyl) sulfanyl-6-[6-(4-fluoro-2- isopropyl-phenoxy)-2- pyridyl]-6-(3- thienyl)piperidine-2,4- dione was prepared in 12% yield according to method 3. Step A substututing 2-chloro-4-fluorophenol for 4-fluoro-2-isopropyl- phenol | 1H NMR (400 MHz, METHANOL- d4) d = 7.87 (dd, J = 8.0, 8.0 Hz, 1H), 7.41 (dd, J = 3.6, 1.2 Hz, 1H), 7.32 (d, J = 7.6, 1H), 7.26 (d, J = 7.6, 1H), 7.18-7.17 (m, 2H), 7.05-6.90 (m, 5H), 6.73 (dd, J = 8.0, 8.0 Hz, 1H), 5.87 (d, J = 8.0, 1H), 3.40 (d, J = 16.4 Hz, 1H), 3.12 (d, J = 16.4 Hz, 1H), 2.94-2.90 (m, 1H), 1.02 (d, J = 6.8 Hz, 3H). 0.98 (d, J = 6.8 Hz, 3H), |
| 367 | MD | 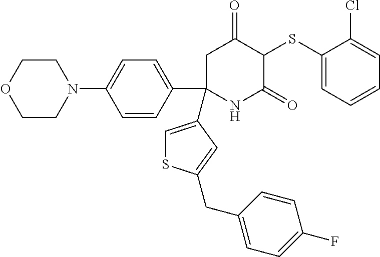 [SMILES: O=C1CC(c2ccc(N3CCOCC3)cc2)(c2csc(Cc3ccc(F)cc3)c2)NC(=O)C1Sc1ccccc1Cl] | 3-(2-chlorophenyl)sulfanyl- 6-[5-[(4-fluorophenyl) methyl]-3-thienly]-6-(4- morpholinophenyl) piperidine-2,4-dione was prepared according to methods described therein. | 1H NMR (400 MHz, METHANOL- d4) d = 8.31 (s, 1H), 7.25-7.20 (m, 5H), 7.05 (dd, J = 8.4, 8.4 1H), 7.03 (s, 1H), 6.92-6.91 (m, 4H), 6.60 (dd, J = 7.2, 7.2 1H)), 4.07 (s, 2H), 3.71 (dd, J = 4.4, 4.4 Hz, 4H), 3.32 (s, 2H), 3.09 (dd, J = 9.2, 4.4 Hz, 4H) |
| 368 | MD | 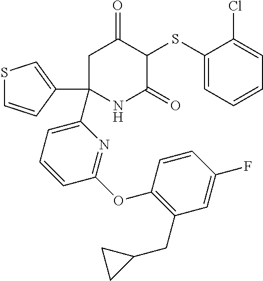 [SMILES: O=C1CC(c2ccsc2)(c2cccc(Oc3ccc(F)cc3CC3CC3)n2)NC(=O)C1Sc1ccccc1Cl] | 3-(2-chlorophenyl)sulfanyl- 6-[6-[2-(cyclopropyl- methyl)-4-fluoro- phenoxy]-2-pyridyl]-6- (3-thienyl)piperidine- 2,4-dione was prepared in 13% yield according to the Method 3, Step A substituting 2-chloro-4- fluorophenol for 2-(cyclopropylmethyl)- 4-fluorophenol | ¹H NMR (400 MHz, DMSO-d₆) δ = 7.88 (dd, J = 8 Hz, 8 Hz, 1H), 7.37 (dd, J = 4.2, 4.2 Hz, 1H), 7.31 (d, J = 8.0 Hz 1H), 7.23-7.21 (m, 1H), 7.13 (s, 1H), 7.00-6.97 (m, 5H), 6.78 (dd, J = 8,0, 8.0 Hz, 1H), 5.96 (d, J = 8.0 Hz, 1H), 3.55 (d, J = 16 Hz, 1H), 3.23 (d, J = 16 Hz, 1H), 2.30 (d, J = 6.8 Hz, 2H), 0.88-0.82 (m, 1H), 0.38- 0.36 (m. 2H), 0.03-0.02 (m, 2H) |
| 369 | MD | 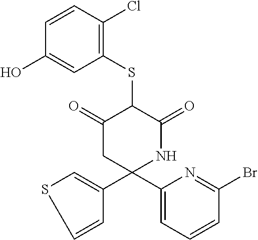 [SMILES: O=C1CC(c2ccsc2)(c2cccc(Br)n2)NC(=O)C1Sc1cc(O)ccc1Cl] | 6-(6-bromo-2-pyridyl)-3- (2-chloro-5-hydroxy- phenyl)sulfanyl-6-(3- thienyl)piperidine-2,4- dione | ¹H NMR (400 MHz, DMSO-d6) δ 11.70 (s, 1H), 9.43 (s, 1H), 8.55 (s, 1H), 7.82 (t, J = 7.8 Hz, 1H), 7.67 (d, J = 7.6 Hz, 1H), 7.59 (d, J = 7.6 Hz, 1H), 7.53 (dd, J = 5.1, 3.0 Hz, 1H), 7.34 (dd, J = 3.0, 1.4 Hz, 1H), 7.17-7.01 (m, 2H), 6.42 (dd, J = 8.6, 2.8 Hz, 1H), 5.91 (d, J = 2.7 Hz, 1H), 3.90 (d, J = 16.6 Hz, 1H), 3.39 (d, J = 16.5 Hz, 1H). |
| 370 | SS | 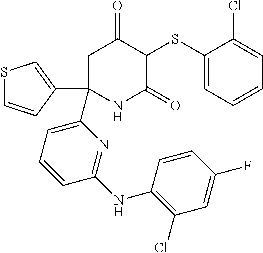 [SMILES: O=C1CC(c2ccsc2)(c2cccc(Nc3ccc(F)cc3Cl)n2)NC(=O)C1Sc1ccccc1Cl] | (6R)-6-[6-(2-chloro-4- fluoro-anilino)-2-pyridyl]- 3-(2-chlorophenyl) sulfanyl-6-(3-thienyl) piperidine-2,4-dione was prepared in 5.6% yield according to the Example 4, Step A substituting cyclohexanamine for (2-chloro-4-fluoro- benzenamine | 1H NMR (400 MHz, METHANOL- d4) δ = 8.44 (s, 1H), 7.93 (dd, J = 5.2, 5.2 Hz, 1H), 7.50 (dd, J = 8.0, 8.0 Hz, 1H), 7.43-7.33 (m, 2H), 7.31 (s, 1H), 7.21 (d, J = 8.0 Hz, 1H), 7.10-7.06 (m, 3H), 6.88-6.85 (m, 2H), 6.73 (dd, J = 5.2, 5.2 Hz, 1H), 6.06 (d, J = 7.6 Hz, 1H), 3.75 (s, 2H) |
| 371 | MD | 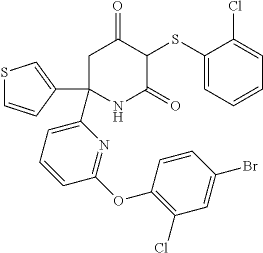 [SMILES: O=C1CC(c2ccsc2)(c2cccc(Oc3ccc(Br)cc3Cl)n2)NC(=O)C1Sc1ccccc1Cl] | 6-[6-(4-bromo-2-chloro- phenoxy)-2-pyridyl]-3- (2-chlorophenyl)sulfanyl- 6-(3-thienyl)piperidine- 2,4-dione was prepared in 1.9% yield according to the Example 3, Step A substituting 2-chloro-4- fluorophenol for 4-bromo- 2-chlorophenol | 1H NMR (400 MHz, METHANOL- d4) δ = 7.91 (dd, J = 8.0, 8.0 Hz, 1H), 7.69 (s, 1H), 7.47 (d, J = 3.2 Hz, 1H), 7.36 (dd, J = 4.0, 4.0 Hz, 2H), 7.20 (d, J = 4.8 Hz, 1H), 7.11-7.08 (m, 3H), 7.06-6.95 (m, 2H), 6.70 (dd, J = 5.2, 5.2 Hz, 1H), 5.96 (d, J = 5.2 Hz, 1H) , 3.49 (d, J = 16.4 Hz, 1H), 3.21 (d, J = 16.4 Hz, 1H) |
| 372 | SS | 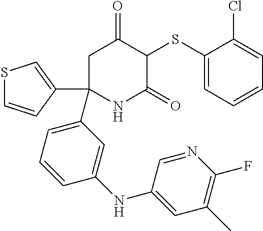 [SMILES: Cc1cc(Nc2cccc(C3(c4ccsc4)CC(=O)C(Sc4ccccc4Cl)C(=O)N3)c2)cnc1F] | (6S)-3-(2-chlorophenyl) sulfanyl-6-[3-[(6-fluoro- 5-methyl-3-pyridyl)amino] phenyl]-6-(3-thienyl) piperidine-2,4-dione was prepared in 6% yield according to example 335 substituting6-(6-bromo- pyridin-2-yl)-3-((2-chloro- phenyl)thio)-6-(thiophen- 3-yl)piperidine-2,4-dione for 6-(3-bromophenyl)-3- (2-chlorophenyl)thio)-6- (thiophen-3-yl)piperidine- 2,4-dione. | 1H NMR (400 MHz, METHANOL- d4) d = 8.26 (s, 1H), 7.66 (s, 1H), 7.52 (dd, J = 4.4, 3.2 Hz, 1H), 7.33-7.32 (m, 2H), 7.23- 7.21 (m, 2H), 7.14 (d, J = 5.2, 1H), 7.07 (s, 1H), 6.90-6.89 (m, 3H), 6.70 (dd, J = 8.0, 8.0 Hz, 1H), 5.95 (d, J = 7.6, 1H), 3.28 (s, 2H), 2.11 (s, 3H) |
| 373 | MD | 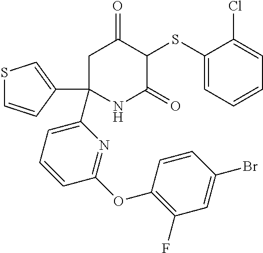 [SMILES: O=C1CC(c2ccsc2)(c2cccc(Oc3ccc(Br)cc3F)n2)NC(=O)C1Sc1ccccc1Cl] | 6-[6-(4-bromo-2-fluoro- phenoxy)-2-pyridyl]-3- (2-chlorophenyl)sulfanyl- 6-(3-thienyl)piperidine- 2,4-dione was prepared in 8% yield according to the Example 3, Step A substituting 2-chloro-4- fluorophenol for 4-bromo- 2-fluorophenol | ¹H NMR (400 MHz, DMSO-d₆) δ = 7.91 (dd, J = 8 Hz, 8 Hz, 1H), 7.46 (d, J = 8.0 Hz 1H), 7.38- 7.35 (m, 3H), 7.22 (d, J = 8.0 Hz, 1H), 7.13-7.08 (m, 3H), 6.98-6.97 (m, 2H), 6.79 (dd, J = 8.0H, 8.0 Hz, 1H), 5.98 (d, J = 8.0 Hz, 1H), 3.53 (d, J = 16 Hz, 1H), 3.25 (d, J = 16 Hz, 1H) |
| 374 | SS | 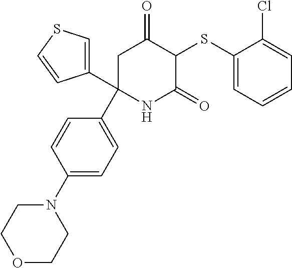 [SMILES: O=C1CC(c2ccc(N3CCOCC3)cc2)(c2ccsc2)NC(=O)C1Sc1ccccc1Cl] | 5-(2-chlorophenyl)sulfanyl- 4-hydroxy-2-(4- morpholinophenyl)-2- (3-thienyl)-1,3-dihydro- pyridin-6-one was prepared in 4% yield according to the Method 7, Step H substituting 2-methyl- morpholine for morpholine | ¹H NMR (400 MHz, DMSO-d₆) δ = 8.40 (s, 1H), 7.75 (dd, J = 8.0, 8.0 Hz, 1H), 7.31-7.23 (m, 4H), 7.14 (d, J = 8.0 Hz, 1H), 6.95-6.93 (m, 3H), 6.75 (dd, J = 8.0 8.0 Hz, 1H), 5.88 (d, J = 8 Hz 1H), 3.73 (t, J = 4.4 Hz, 4H), 3.48 (d, J = 16 Hz 1H), 3.28 (d, J = 16 Hz, 1H), 3.11 (t, J = 4.4 Hz 4H) |
| 375 | SS | 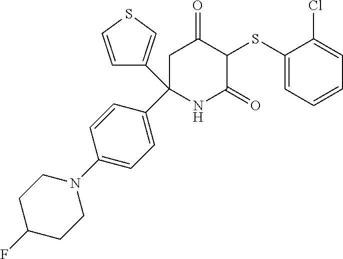 [SMILES: O=C1CC(c2ccc(N3CCC(F)CC3)cc2)(c2ccsc2)NC(=O)C1Sc1ccccc1Cl] | 5-(2-chlorophenyl) sulfanyl-2-[4-(4-fluoro- 1-piperidyl)phenyl]-4- hydroxy-2-(3-thienyl)- 1,3-dihydropyridin- 6-one | |
| 376 | MD | 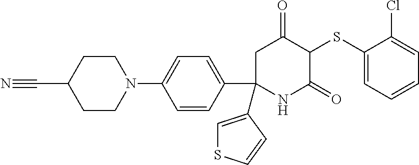 [SMILES: N#CC1CCN(c2ccc(C3(c4ccsc4)CC(=O)C(Sc4ccccc4Cl)C(=O)N3)cc2)CC1] | 1-[4-[5-(2-chlorophenyl) sulfanyl-4,6-dioxo-2- (3-thienyl)-2-piperidyl] phenyl]piperidine-4- carbonitrile was prepared in 3% yield according to the Method 7, Step H substituting 2-methyl- morpholine for cyanozinc. | 1H NMR (400 MHz, METHANOL- d4) δ = 7.43 (dd, J = 7.6, 2.4 Hz, 1H), 7.30 (d, J = 8.8 Hz, 2H), 7.23 (d, J = 3.2 Hz, 1H), 7.13-7.10 (m, 2H), 6.97 (d, J = 8.8 Hz, 2H), 6.82 (dd, J = 7.6, 7.6 Hz, 1H), 6.70 (dd, J = 7.6, 7.6 Hz, 1H), 6.05 (d, J = 7.6 Hz, 1H), 3.42 (t, J = 5.2 Hz, 2H), 3.28 (s, 2H), 3.09 (t, J = 5.2 Hz, 2H), 3.01-2.94 (m, 1H), 2.03 (t, J = 5.2 Hz, 2H), 1.92 (t, J = 5.2 Hz, 2H) |
| 377 | MD | 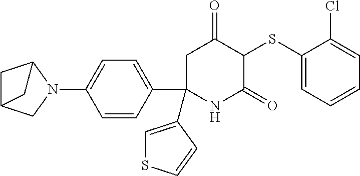 [SMILES: O=C1CC(c2ccc(N3CC4CC3C4)cc2)(c2ccsc2)NC(=O)C1Sc1ccccc1Cl] | 6-[4-(3-azabicyclo[2.1.1] hexan-3-yl)phenyl]-3- (2-chlorophenyl)sulfanyl- 6-(3-thienyl)piperidine- 2,4-dione was prepared in 7% yield according to the Method 7, Step H substututing 2-methyl- morpholine for 2- azabicyclo[2.1.1]hexane. | 1H NMR (400 MHz, METHANOL- d4) δ = 7.58 (dd, J = 5.2, 5.2 Hz, 1H), 7.54 (d, J = 8.8 Hz, 2H), 7.46 (d, J = 8.8 Hz, 2H), 7.36 (d, J = 3.2 Hz, 1H), 7.22 (d, J = 8.8 Hz, 1H), 7.21 (d, J = 3.8 Hz, 1H), 6.94 (dd, J = 7.6, 7.6 Hz, 1H), 6.82 (dd, J = 7.6, 7.6 Hz, 1H), 6.08 (dd, J = 4.8, 1.2 Hz, 1H), 4.70-4.68 (m, 1H), 3.85 (d, J = 3.6 Hz, 2H), 3.49 (dd, J = 16.0, 16.0 Hz, 2H), 3.08-3.06 (m, 1H), 2.31 (dd, J = 2.4, 2.4 Hz, 2H), 1.73 (dd, J = 2.0, 2.0 Hz, 2H) |
| 378 | SS | 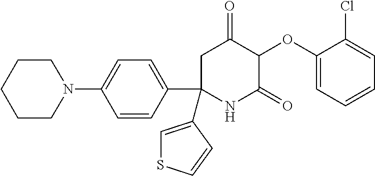 [SMILES: O=C1CC(c2ccc(N3CCCCC3)cc2)(c2ccsc2)NC(=O)C1Oc1ccccc1Cl] | (6S)-3-(2-chlorophenoxy)- 6-[4-(1-piperidyl)phenyl]- 6-(3-thienyl)piperidine- 2,4-dione was prepared in 8% yield according to the example 37, step C substituting morpholine for piperidine | 1H NMR (400 MHz, Methanol-d6) δ 7.46 (dd, J = 5.2, 3.2 Hz, 1H), 7.31-7.27 (m, 4H), 7.13 (d, J = 4.0 Hz, 1H), 7.00 (d, J = 8 Hz, 2H), 6.82-6.79 (m, 2H), 5.94 (dd, J = 8.0, 4.0 Hz, 1H), 3.34- 3.32 (m, 2H), 3.19-3.17 (m, 4H), 1.82-1.70 (m, 4H), 1.62-1.60 (m, 2H) |
| 379 | MD | 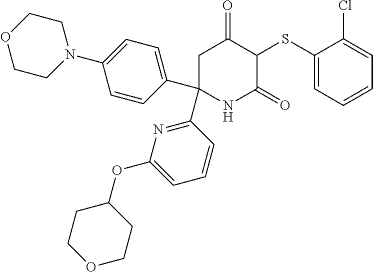 [SMILES: O=C1CC(c2ccc(N3CCOCC3)cc2)(c2cccc(OC3CCOCC3)n2)NC(=O)C1Sc1ccccc1Cl] | 3-(2-chlorophenyl)sulfanyl- 6-(4-morpholinophenyl)- 6-(6-tetrahydro-pyran-4- yloxy-2-pyridyl) piperidine-2,4-dione was prepared in 14% yield according to the Method 41, Step A substituting cyclohexanol for tetra- hydro-2H-pyran-4-ol | ¹H NMR (400 MHz, DMSO-d₆) δ = 7.80-7.72 (m, 3H), 7.65-7.63 (m, 2H), 7.24 (d, J = 8.0 Hz, 1H), 7.18 (d, J = 8.0 Hz, 1H), 6.97 (dd, J = 8.0 8.0 Hz, 1H), 6.84 (d, J = 8.0 Hz, 1H), 6.76 (dd, J = 8.0, 8.0 Hz, 1H), 5.94 (d, J = 8.0 Hz, 1H), 5.32-5.27 (m, 1H), 4.11 (t, J = 4.8 Hz, 4H), 3.93-3.88 (m, 3H), 3.71 (t, J = 4.8 Hz, 4H), 3.54-3.50 (m, 2H), 3.42 (d, J = 16 Hz, 1H), 2.06-2.05 (m, 1H), 1.93-1.91 (m, 1H), 1.76-1.73 (m, 1H), 1.60-1.57 (m, 1H) |
| 380 | MD | 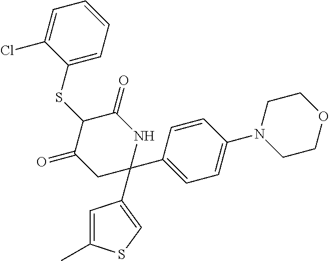 [SMILES: Cc1cc(C2(c3ccc(N4CCOCC4)cc3)CC(=O)C(Sc3ccccc3Cl)C(=O)N2)cs1] | 3-(2-chlorophenyl)sulfanyl- 6-(5-methyl-3-thienyl)- 6-(4-morpholinophenyl) piperidine-2,4-dione was prepared according to methods described therein. | ¹H NMR (400 MHz, DMSO-d₆) δ 11.37 (s, 1H), 8.27 (s, 1H), 7.32- 7.21 (m, 3H), 7.04 (d, J = 1.6 Hz, 1H), 6.99-6.88 (m, 3H), 6.86-6.71 (m, 2H), 5.97 (dd, J = 7.9, 1.4 Hz, 1H), 3.74 (dd, J = 5.9, 3.7 Hz, 4H), 3.33 (d, J = 5.4 Hz, 2H), 3.18-3.04 (m, 4H), 2.42 (d, J = 1.0 Hz, 3H). |
| 381 | SS | 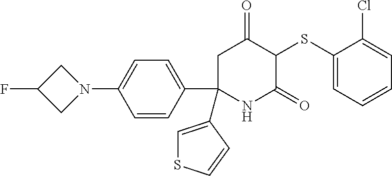 [SMILES: O=C1CC(c2ccc(N3CC(F)C3)cc2)(c2ccsc2)NC(=O)C1Sc1ccccc1Cl] | 3-(2-chlorophenyl)sulfanyl- 6-[4-(8-oxa-3-azabicyclo [3.2.1]octan-1-yl) phenyl]-6-(3-thienyl) piperidine-2,4-dione was prepared in 9% yield according to the Method 7, Step H substituting 2-methylmorpholine for 3-fluoroazetidine | 1H NMR (400 MHz, Methanol-d6) δ 7.46 (dd, J = 5.2, 3.2 Hz, 1H), 7.27-7.11 (m, 5H), 6.88 (dd, J = 7.6, 7.6 Hz, 1H), 6.73 (dd, J = 7.6, 7.6 Hz, 1H), 6.52 (d, J = 8.0 Hz, 2H), 5.99 (d, J = 8.0 Hz, 1H), 5.42 (d, J = 17.2 Hz, 1H), 4.22-4.14 (m, 2H), 3.93-3.85 (m, 2H), 3.35 (s, 2H) |
| 382 | MD | 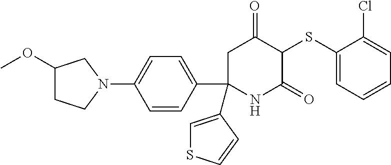 [SMILES: COC1CCN(c2ccc(C3(c4ccsc4)CC(=O)C(Sc4ccccc4Cl)C(=O)N3)cc2)C1] | 3-(2-chlorophenyl)sulfanyl- 6-[4-(3-methoxy- pyrrolidin-1-yl)phenyl]- 6-(3-thienyl)piperidine- 2,4-dione was prepared in 4.5% yield according to example 7, Step H substituting 2-methyl- morpholine for 3- methoxypyrrolidine | 1H NMR (400 MHz, METHANOL- d4) δ = 7.45 (dd, J = 5.2, 3.2 Hz, 1H), 7.24 (d, J = 9.2 Hz, 2H), 7.23-7.20 (m, 1H), 7.15 (d, J = 8.0 Hz, 1H), 7.13 (d, J = 4.8 Hz, 1H), 6.85 (dd, J = 7.6, 7.6 Hz, 1H), 6.66 (dd, J = 7.2, 7.2 Hz, 1H), 6.57 (d, J = 8.4 Hz, 2H), 5.95 (d, J = 7.2 Hz, 1H), 4.13 (d, J = 2.8 Hz, 1H), 3.48 (dd, J = 10.87, 5.2 Hz, 1H), 3.36-3.37 (m, 8H), 2.16-2.11 (m, 2H) |
| 383 | MD | 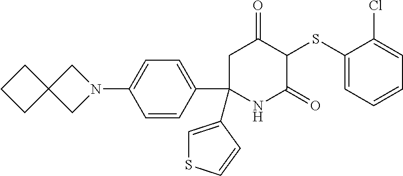 [SMILES: O=C1CC(c2ccc(N3CC4(CCC4)C3)cc2)(c2ccsc2)NC(=O)C1Sc1ccccc1Cl] | 6-[4-(2-azaspiro[3.3] heptan-2-yl)phenyl]-3- (2-chlorophenyl)sulfanyl- 6-(3-thienyl)piperidine- 2,4-dione was prepared in 5.2% yield according to example 7, Step H substituting 2-methyl- morpholine for 2- azaspiro[3.3]heptane | 1H NMR (400 MHz, METHANOL- d4) δ = 7.46 (dd, J = 5.2, 2.8 Hz, 1H), 7.24-7.21 (m, 3H), 7.18 (d, J = 7.2 Hz, 1H), 7.12 (d, J = 4.8 Hz, 1H), 6.88 (dd, J = 7.2, 6.4 Hz, 1H), 6.72 (dd, J = 7.2, 7.2 Hz, 1H), 6.47 (d, J = 8.4 Hz, 2H), 5.96 (d, J = 7.2 Hz, 1H), 3.37 (s, 2H), 3.80 (s, 4H), 2.22 (t, J = 7.2 Hz, 4H), 1.92- 1.85 (m, 2H) |
| 384 | MD | 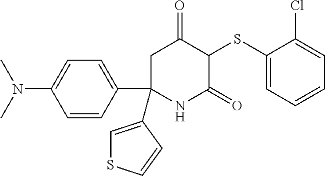 [SMILES: CN(C)c1ccc(C2(c3ccsc3)CC(=O)C(Sc3ccccc3Cl)C(=O)N2)cc1] | 3-(2-chlorophenyl)sulfanyl- 6-[4-dimethylamino) phenyl]-6-(3-thienyl) piperidine-2,4-dione was prepared in 1.6% yield according to example 7, Step H substituting 2- methylmorpholine for dimethylamine | 1H NMR (400 MHz, METHANOL- d4) δ = 7.45 (dd, J = 5.2, 3.2 Hz, 1H), 7.25 (d, J = 8.8 Hz, 2H), 7.23-7.21 (m, 1H), 7.16-7.13 (m, 2H), 6.86 (dd, J = 7.2, 6.4 Hz, 1H), 6.77 (d, J = 8.8 Hz, 2H), 6.68 (dd, J = 8.0, 8.8 Hz, 1H), 5.99 (d, J = 8.4 Hz, 1H), 3.37 (s, 2H), 2.94 (s, 6H) |
| 385 | SS | 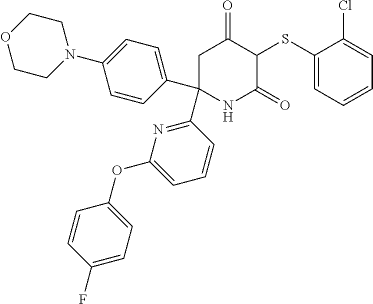 [SMILES: O=C1CC(c2ccc(N3CCOCC3)cc2)(c2cccc(Oc3ccc(F)cc3)n2)NC(=O)C1Sc1ccccc1Cl] | (6S)-3-(2-chlorophenyl) sulfanyl-6-[6-(4-fluoro- phenoxy)-2-pyridyl]- 6-(4-morpholinophenyl) piperidine-2,4-dione was prepared in 13% yield according to the Method 40, Step A substituting 3,4-difluorophenol for 4-fluorophenol | ¹H NMR (400 MHz, DMSO-d₆) δ = 7.87 (dd, J = 8 Hz, 8 Hz, 1H), 7.31 (d, J = 8.0 Hz, 1H), 7.20-7.18 (m, 3H), 7.10-7.06 (m, 4H), 6.98- 6.93 (m, 4H), 6.73 (dd, J = 8.0 8.0 Hz, 1H), 5.95 (d, J = 8.0 Hz, 1H), 3.56 (d, J = 16 Hz 1H), 3.28 (d, J = 16 Hz, 1H), 3.14 (t, J = 4.8 Hz, 4H) |
| 386 | SS | 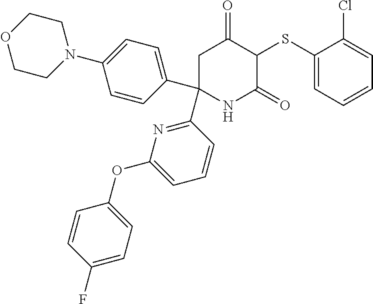 [SMILES: O=C1CC(c2ccc(N3CCOCC3)cc2)(c2cccc(Oc3ccc(F)cc3)n2)NC(=O)C1Sc1ccccc1Cl] | (6R)-3-(2-chlorophenyl) sulfanyl-6-[6-(4-fluoro- phenoxy)-2-pyridyl]- 6-(4-morpholinophenyl) piperidine-2,4-dione was prepared in 15% yield according to example 40, Step A substituting 3,4-difluorophenol for 4-fluorophenol | ¹H NMR (400 MHz, DMSO-d₆) 7.84 (dd, J = 8.0, 8.0 Hz, 1H), 7.29 (d, J = 8.0 Hz, 1H), 7.19- 7.17 (m, 3H), 7.09-7.05 (m, 4H), 6.96-6.89 (m, 4H), 6.71 (dd, J = 8.0, 8.0 Hz, 1H), 5.94 (d, J = 8.0 Hz, 1H), 3.81 (t, J = 4.8 Hz, 1H), 3.53 (d, J = 16 Hz, 1H), 3.26 (d, J = 16 Hz, 1H), 3.12 (t, J = 4.8 Hz, 1H) |
| 387 | SS | 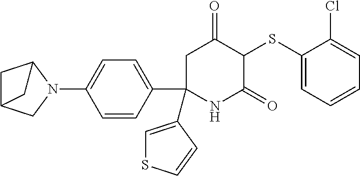 [SMILES: O=C1CC(c2ccc(N3CC4CC3C4)cc2)(c2ccsc2)NC(=O)C1Sc1ccccc1Cl] | (6R)-6-[4-(3-azabicyclo [2.1.1]hexan-3-yl)phenyl]- 3-(2-chlorophenyl) sulfanyl-6-(3-thienyl) piperidine-2,4-dione was prepared in 7% yield according to example 7, Step H substituting 2- methylmorpholine for 2-azabicyclo[2.1.1] hexane. | 1H NMR (400 MHz, METHANOL- d4) δ = 7.46 (dd, J = 2.0, 5.2 Hz, 1H), 7.25-7.23 (m, 3H), 7.22 (d, J = 8.8 Hz, 1H), 7.17 (d, J = 3.2, 1.2 Hz, 1H), 7.12 (dd, J = 4.8, 4.8 Hz, 1H), 6.79- 6.75 (m, 3H), 6.04 (d, J = 7.6 Hz, 1H), 4.38-4.37 (m, 1H), 3.37 (d, J = 3.6 Hz, 2H), 3.3 (d, J = 16.0 Hz, 2H), 2.93-2.92 (m, 1H), 1.96 (dd, J = 2.4, 2.4 Hz, 2H), 1.38 (dd, J = 2.0, 2.0 Hz, 2H) |
| 388 | MD | 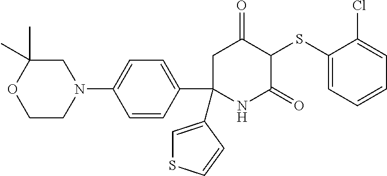 [SMILES: CC1(C)CN(c2ccc(C3(c4ccsc4)CC(=O)C(Sc4ccccc4Cl)C(=O)N3)cc2)CCO1] | 3-(2-chlorophenyl)sulfanyl- 6-[4-(2,2-dimethyl- morpholin-4-yl)phenyl]- 6-(3-thienyl)piperidine- 2,4-dione was prepared in 4% yield according to example 7, Step H substituting 2-methyl- morpholine for 2,2- dimethylmorpholine. | 1H NMR (400 MHz, MeOD-d4) δ 7.48 (dd, J = 5.2 Hz, 5.2 Hz, 1H), 7.33 (d, J = 8.8 Hz, 2H), 7.24 (d, J = 3.2 Hz, 1H), 7.18 (d, J = 8.8 Hz 1H), 7.15 (d, J = 3.2 Hz, 1H), 7.14 (d, J = 8.8 Hz, 2H), 7.01 (dd, J = 7.6 Hz, 7.6 Hz, 1H), 6.75 (dd, J = 7.6 Hz, 7.6 Hz 1H), 5.94 (dd, J = 7.6 Hz, 7.6 Hz 1H), 3.88 (t, J = 4.8 Hz, 2H), 3.40 (s, 2H), 3.16 (t, J = 4.8 Hz, 2H), 3.03 (s, 2H), 1.32 (s. 6H). |
| 389 | SS | 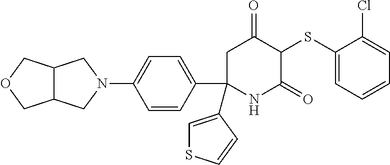 [SMILES: O=C1CC(c2ccc(N3CC4COCC4C3)cc2)(c2ccsc2)NC(=O)C1Sc1ccccc1Cl] | (6S)-6-[4-(1,3,3a,4,6,6a- hexahydrofuro[3,4-c] pyrrol-5-yl)phenyl]-3-(2- chlorophenyl)sulfanyl-6- (3-thienyl)piperidine- 2,4-dione was prepared in 4% yield according to example 7, Step H substituting 2-methyl- morpholine for hexahydro-1H-furo[3,4- c]pyrrole. | 1H NMR (400 MHz, MeOD-d4) δ 7.48 (dd, J = 5.2 Hz, 5.2 Hz, 1H), 7.28 (d, J = 8.8 Hz, 2H), 7.23 (d, J = 3.2 Hz, 1H), 7.23 (d, J = 3.2 Hz, 1H), 7.20 (d, J = 3.2 Hz, 1H), 7.13 (d, J = 3.2 Hz, 1H), 6.76 (dd, J = 4.8 Hz, 4.8 Hz, 2H), 6.73 (d, J = 8.8 Hz, 2H), 6.71 (dd, J = 7.6 Hz, 7.6 Hz, 1H), 5.93 (d, J = 7.6 Hz, 1H), 3.93 (dd, J = 2.0 Hz, 2.0 Hz, 1H), 3.67 (dd, J = 2.0 Hz, 2.0 Hz, 1H), 3.47-3.42 (m, 4H), 3.26 (d, J = 2.0 Hz, 2H), 3.09 (d, J = 2.0 Hz, 2H). |
| 390 | SS | 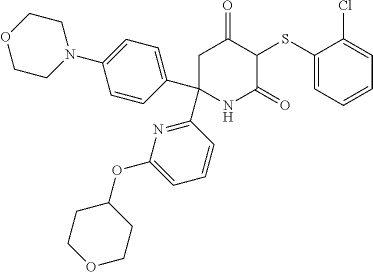 [SMILES: O=C1CC(c2ccc(N3CCOCC3)cc2)(c2cccc(OC3CCOCC3)n2)NC(=O)C1Sc1ccccc1Cl] | (6S)-3-(2-chlorophenyl) sulfanyl-6-(4-morpholino- phenyl)-6-(6-tetrahydro- pyran-4-yloxy-2-pyridyl) piperidine-2,4-dione was prepared in 6% yield according to example 41, Step A substituting cyclo- hexanol for tetrahydro- 2H-pyran-4-ol | ¹H NMR (400 MHz, Methanol-d₄) δ = 7.73 (dd, J = 8 Hz, 8 Hz, 1H), 7.23-7.13 (m, 4H), 6.9-6.92 (m, 3H), 6.76 (d, J = 8.0 Hz, 1H), 6.67 (dd, J = 8.0 8.0 Hz, 1H), 5.90 (d, J = 8.0 Hz, 1H), 5.31-5.26 (m, 1H), 3.94-3.81 (m, 5H), 3.76 (d, J = 16 Hz, 1H), 3.70-3.60 (m, 3H), 3.39 (d, J = 16 Hz, 1H), 3.15-3.13 (d, J = 16 Hz, 1H), 2.40-2.38 (m, 4H), 2.05-1.92 (m, 2H), 1.74-1.71 (m, 1H), 1.61-1.58, (m, 1H) |
| 391 | SS | 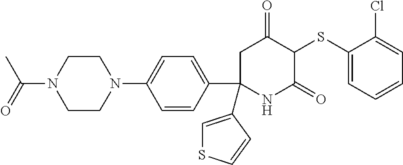 [SMILES: CC(=O)N1CCN(c2ccc(C3(c4ccsc4)CC(=O)C(Sc4ccccc4Cl)C(=O)N3)cc2)CC1] | (6R)-6-[4-(4-acetyl- piperazin-1-yl)phenyl]- 3-(2-chlorophenyl) sulfanyl-6-(3-thienyl) piperidine-2,4-dione | |
| 392 | SS | 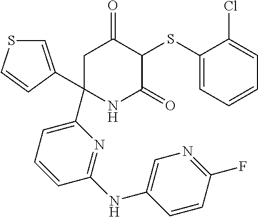 [SMILES: O=C1CC(c2ccsc2)(c2cccc(Nc3ccc(F)nc3)n2)NC(=O)C1Sc1ccccc1Cl] | (6S)-3-(2-chlorophenyl) sulfanyl-6-[6-[(6-fluoro- 3-pyridyl)amino]-2- pyridyl]-6-(3-thienyl) piperidine-2,4-dione was prepared as in example 397 | |
| 393 | SS | 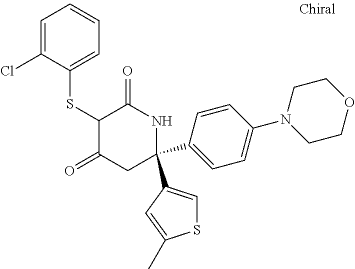 [SMILES: Cc1cc([C@@]2(c3ccc(N4CCOCC4)cc3)CC(=O)C(Sc3ccccc3Cl)C(=O)N2)cs1] | (6S)-3-(2-chlorophenyl) sulfanyl-6-(5-methyl- 3-thienyl)-6-(4-morpho- linophenyl)piperidine- 2,4-dione | |
| 394 | SS | 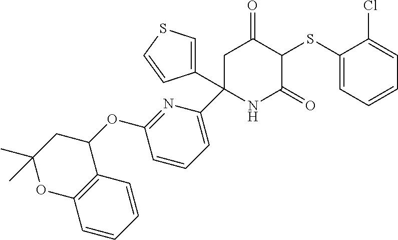 [SMILES: CC1(C)CC(Oc2cccc(C3(c4ccsc4)CC(=O)C(Sc4ccccc4Cl)C(=O)N3)n2)c2ccccc2O1] | 5-(2-chlorophenyl)sulfanyl- 2-[6-(2,2-dimethyl- chroman-4-yl)oxy-2- pyridyl[-4-hydroxy-2- (3-thienyl)-1,3-dihydro- pyridin-6-one | |
| 395 | SS | 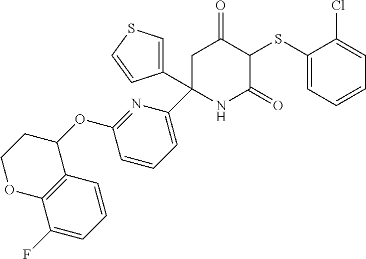 [SMILES: O=C1CC(c2ccsc2)(c2cccc(OC3CCOc4c(F)cccc43)n2)NC(=O)C1Sc1ccccc1Cl] | 5-(2-chlorophenyl)sulfanyl- 2-[6-(8-fluoro- chroman-4-yl)oxy-2- pyridyl]-4-hydroxy-2- (3-thienyl)-1,3-dihydro- pyridin-6-one | |
| 396 | MD | 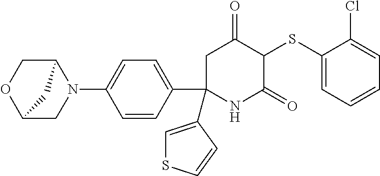 [SMILES: O=C1CC(c2ccc(N3C[C@@H]4C[C@H]3CO4)cc2)(c2ccsc2)NC(=O)C1Sc1ccccc1Cl] | 3-(2-chlorophenyl)sulfanyl- 6-[4-[(1S,4S)-2-oxa-5- azabicyclo[2.2.1]heptan- 5-yl]phenyl]-6-(3-thienyl) piperidine-2,4-dione was prepared in 2% yield according to example 7, Step H substituting 2-methylmorpholine for (1S,4S)-2-oxa-5- azabicyclo[2.2.1]heptane. | 1H NMR (400 MHz, METHANOL- d4) δ = 7.51 (dd, J = 5.2, 5.2 Hz, 1H), 7.29-7.21 (m, 3H), 7.22 (d, J = 7.2 Hz, 1H), 7.17 (d, J = 3.2 Hz, 1H), 6.94 (dd, J = 5.2, 5.2 Hz, 1H), 6.76 (dd, J = 2.0. 2.0 Hz, 1H), 6.67 (d, J = 8.0 Hz, 2H), 4.65 (d, J = 20.0 Hz, 2H), 3.85 (d, J = 2.0 Hz, 2H), 3.60 (d, J = 9.6 Hz, 1H), 3.45 (s, 2H), 3.12 (d, J = 9.2 Hz, 1H), 2.07-1.97 (m, 2H) |
| 397 | SS | 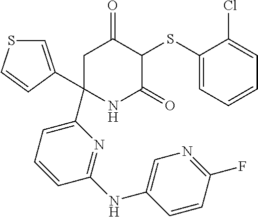 [SMILES: O=C1CC(c2ccsc2)(c2cccc(Nc3ccc(F)nc3)n2)NC(=O)C1Sc1ccccc1Cl] | (6R)-3-(2-chlorophenyl) sulfanyl-6-[6-[(6-fluoro- 3-pyridyl)amino]-2- pyridyl]-6-(3-thienyl) piperidine-2,4-dione was prepared in 18.5% yield according to example 4, Step A substituting cyclo- hexanamine for 6-fluoro- pyridin-3-amine | 1H NMR (400 MHz, METHANOL- d4) δ = 8.33 (s, 1H), 8.21 (dd, J = 3.2, 2.4 Hz, 1H), 7.59 (dd, J = 8.0, 8.0 Hz, 1H), 7.41 (d, J = 2.0 Hz, 1H), 7.30 (s, 1H), 7.14-7.08 (m, 2H), 7.02 (d, J = 7.6 Hz, 1H), 6.86 (d, J = 7.6 Hz, 1H), 6.75 (dd, J = 8.0, 8.0 Hz, 1H), 6.70-6.59 (m, 2H), 6.15 (d, J = 7.6 Hz, 1H), 3.65 (d, J = 16.4 Hz, 1H), 3.40 (d, J = 16.4 Hz, 1H) |
| 398 | SS | 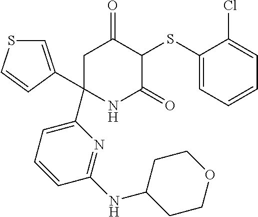 [SMILES: O=C1CC(c2ccsc2)(c2cccc(NC3CCOCC3)n2)NC(=O)C1Sc1ccccc1Cl] | (6R)-3-(2-chlorophenyl) sulfanyl-6-[6-(tetrahydro- pyran-4-ylamino)-2- pyridyl]-6-(3-thienyl) piperidine-2,4-dione was prepared in 24% yield according to example 4, Step A substituting cyclo- hexanamine for tetra- hydro-2H-pyran-4-amine | ¹H NMR (400 MHz, DMSO-d₆) δ = 7.42-7.39 (m, 2H), 7.26-7.13 (m, 3H), 6.91 (dd, J = 8.0 8.0 Hz, 1H), 6.77 (dd, J = 8.0 8.0 Hz, 1H), 6.70 (d, J = 7.6 Hz, 1H), 6.44 (d, J= 8.0, Hz, 1H), 6.04 (d, J = 8.0 Hz, , 1H), 4.08-4.06 (m, 1H), 3.91-3.87 (m, 2H), 3.79 (d, J = 16 Hz, 1H), 3.56-3.53 (m, 2H), 3.39 (d, J = 16 Hz, 1H), 2.40- 2.38 (m, 2H), 1.97-1.90 (m, 2H), 1.51-1.39 (m, 2H) |
| 399 | MD | 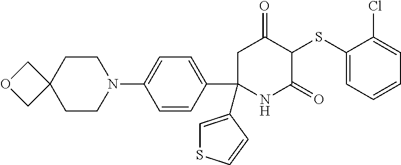 [SMILES: O=C1CC(c2ccc(N3CCC4(CC3)COC4)cc2)(c2ccsc2)NC(=O)C1Sc1ccccc1Cl] | 3-(2-chlorophenyl)sulfanyl- 6-[4-(8-oxa-3-aza- bicyclo[3.2.1]octan-3-yl) phenyl]-6-(3-thienyl) piperidine-2,4-dione was prepared in 5% yield according to example 7, Step H substituting 2- methylmorpholine for 2-oxa-7-azaspiro[3.5] nonane | 1H NMR (400 MHz, Methanol- d6) δ 7.47 (dd, J = 5.2, 3.2 Hz, 1H), 7.29-7.25 (m, 3H), 7.19 (d, J = 8.0 Hz, 1H), 7.13 (d, J = 8.0 Hz, 1H), 6.98 (d, J = 8.8 Hz, 2H), 6.90 (dd, J = 8.0, 8.0 Hz, 1H), 6.73 (dd, J = 8.0, 8.0 Hz, 1H), 5.98 (d, J = 7.6 Hz, 1H), 4.48 (s, 4H), 3.40 (s, 2H), 3.14 (t, J = 5.6 Hz, 4H), 1.99 (t, J = 5.6 Hz, 4H), |
| 400 | MD | 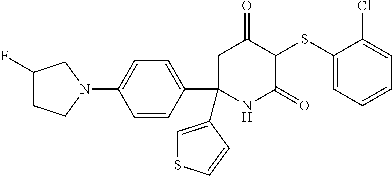 [SMILES: O=C1CC(c2ccc(N3CCC(F)C3)cc2)(c2ccsc2)NC(=O)C1Sc1ccccc1Cl] | 3-(2-chlorophenyl)sulfanyl- 6-[4-(8-oxa-3-aza- bicyclo[3.2.1]octan-3-yl) phenyl]-6-(3-thienyl) piperidine-2,4-dione was prepared in 4% yield according to the Method 7, Step H substituting 2- methylmorpholine for 3-fluoropyrrolidine | 1H NMR (400 MHz, Methanol- d6) δ 7.43 (dd, J = 4.8, 2.8 Hz, 1H), 7.29-7.23 (m, 3H), 7.13-7.11 (m, 2H), 6.81 (dd, J = 8.0, 8.0 Hz, 1H), 6.64-6.59 (m, 3H), 6.02 (d, J = 7.6 Hz, 1H), 5.29 (d, J = 13.6 Hz, 1H), 3.62-3.40 (m, 5H), 3.28 (d, J = 16 Hz, 1H), 2.33- 2.15 (m, 2H) |
| 401 | MD | 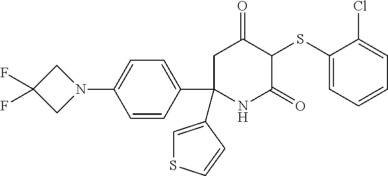 [SMILES: O=C1CC(c2ccc(N3CC(F)(F)C3)cc2)(c2ccsc2)NC(=O)C1Sc1ccccc1Cl] | 3-(2-chlorophenyl)sulfanyl- 6-[4-(3,3-difluoroazetiden- 1-yl)phenyl]-6-(3- thienyl)piperidine-2,4- dione was prepared in 6.7% yield according to the Method 7, Step A substituting 2-methyl- morpholine for 3,3- difluoroazetidine | 1H NMR (400 MHz, METHANOL- d4) δ = 7.48 (dd, J = 2.8, 2.8 Hz, 1H), 7.30 (d, J = 4.8 Hz, 2H), 7.26 (d, J = 3.2 Hz, 1H), 7.25 (d, J = 1.2 Hz, 1H), 7.17 (d, J = 10.4 Hz, 1H), 6.92 (dd, J = 5.2, 5.2 Hz, 1H), 6.70 (dd, J = 5.2, 5.2 Hz, 1H), 6.60 (d, J = 8.8 Hz, 2H), 5.96 (d, J = 6.8 Hz, 1H), 4.22 (t, J = 12.0 Hz, 4H), 3.36 (s, 2H) |
| 402 | MD | 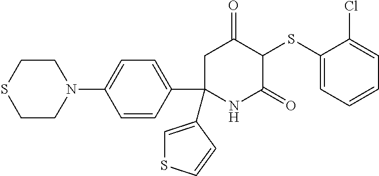 [SMILES: O=C1CC(c2ccc(N3CCSCC3)cc2)(c2ccsc2)NC(=O)C1Sc1ccccc1Cl] | 3-(2-chlorophenyl)sulfanyl- 6-(3-thienyl)-6-(4- thiomorpholinophenyl) piperidine-2,4-dione was prepared in 1% yield according to example 7, Step H substituting 2- methylmorpholine for thiomorpholine. | 1H NMR (400 MHz, METHANOL- d4) δ = 7.45 (dd, J = 7.6, 2.4 Hz, 1H), 7.30 (d, J = 8.8 Hz, 2H), 7.26 (d, J = 3.2 Hz, 1H), 7.18-7.13 (m, 2H), 6.94 (d, J = 8.0 Hz, 2H), 6.82 (dd, J = 7.6, 7.6 Hz, 1H), 6.73 (dd, J = 7.6, 7.6 Hz, 1H), 6.04 (d, J = 7.6 Hz, 1H), 3.57 (t, J = 5.2 Hz, 4H), 3.32 (s, 2H), 2.72 (t, J = 5.2 Hz, 1H) |
| 403 | MD | 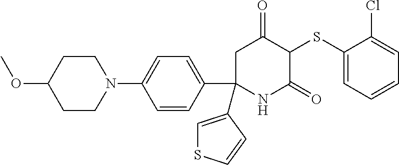 [SMILES: COC1CCN(c2ccc(C3(c4ccsc4)CC(=O)C(Sc4ccccc4Cl)C(=O)N3)cc2)CC1] | 3-(2-chlorophenyl)sulfanyl- 6-[4-(4-methoxy-1- piperidyl)phenyl]-6-(3- thienyl)piperidine-2,4- dione was prepared in 24% yield according to the Method 4, Step A substituting cyclo- hexanamine for 4- methoxypiperidine | ¹H NMR (400 MHz, DMSO-d₆) δ = 7.60-7.55 (m, 5H), 7.37 (dd, J = 4.2, 4.2 Hz, 1H) 7.24 (d, J = 8.0 Hz, 1H), 7.17 (d, J = 4.0 Hz, 1H), 6.96 (dd, J = 8.0, 8.0 Hz, 1H), 6.10 (d, J = 8.0 Hz, 1H), 3.76-3.74 (m, 2H), 3.57-3.55 (m, 1H), 3.52-3.45 (m, 4H), 3.42 (s, 3H), 2.24-2.06 (m, 4H) |
| 404 | MD | 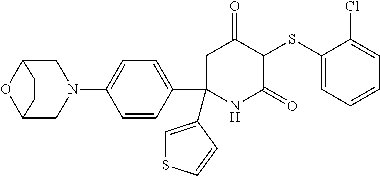 [SMILES: O=C1CC(c2ccc(N3CC4CCC(C3)O4)cc2)(c2ccsc2)NC(=O)C1Sc1ccccc1Cl] | 3-(2-chlorophenyl)sulfanyl- 6-[4-(8-oxa-3-aza- bicyclo[3.2.1]octan-3- yl)phenyl]-6-(3-thienyl) piperidine-2,4-dione was prepared in 6% yield according to the Method 7, Step H substituting 2-methylmorpholine for 8-oxa-3-azabicyclo[3.2.1] octane | 1H NMR (400 MHz, Methanol-d6) δ 7.46 (dd, J = 4.8, 2.8 Hz, 1H), 7.28 (d, J = 8.8 Hz, 2H), 7.21-7.13 (m, 3H), 6.88-6.86 (m, 3H), 6.71 (dd, J = 8.0, 8.0 Hz, 1H), 5.90 (d, J = 8.0 Hz, 1H), 4.46 (s, 2H), 3.45-3.42 (m, 4H), 2.92 (d, J = 7.2 Hz, 2H), 1.95 (s, 2H) |
| 405 | SS | 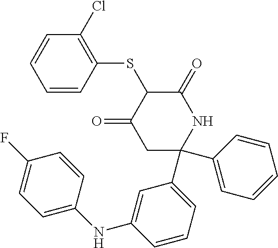 [SMILES: O=C1CC(c2ccccc2)(c2cccc(Nc3ccc(F)cc3)c2)NC(=O)C1Sc1ccccc1Cl] | 3-(2-chlorophenyl)sulfanyl- 6-[3-(4-fluoroanilino) phenyl]-6-phenyl- piperidine-2,4-dione | |
| 406 | SS | 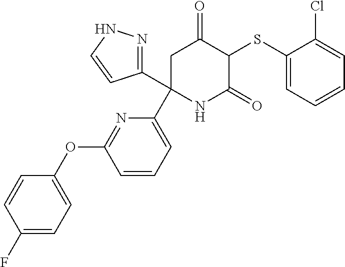 [SMILES: O=C1CC(c2cc[nH]n2)(c2cccc(Oc3ccc(F)cc3)n2)NC(=O)C1Sc1ccccc1Cl] | 5-(2-chlorophenyl)sulfanyl- 2-[6-(4-fluorophenoxy)-2- pyridyl]-4-hydroxy-2- (1H-pyrazol-3-yl)-1,3- dihydropyridin-6-one | |
| 408 | SS | 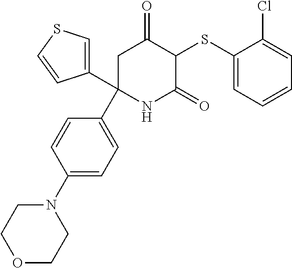 [SMILES: O=C1CC(c2ccc(N3CCOCC3)cc2)(c2ccsc2)NC(=O)C1Sc1ccccc1Cl] | 5-(2-chlorophenyl)sulfanyl- 4-hydroxy-2-(4-morpho- linophenyl)-2-(3- thienyl)-1,3-dihydro- pyridin-6-one was prepared in 55% yield according to the example 7, Step H substituting 2-methyl- morpholine for morpholine | ¹H NMR (400 MHz, DMSO-d₆) δ = 8.38 (s, 1H), 7.57 (dd, J = 8.0, 8.0 Hz, 1H), 7.31-7.25 (m, 4H), 7.23 (d, J = 8.0 Hz, 1H), 6.95- 6.92 (m, 3H), 6.74 (dd, J = 8.0 8.0 Hz, 1H), 5.89 (d, J = 8.0 Hz, 1H), 3.73 (t, J = 4.0 Hz, 4H), 3.48 (d, J = 16 Hz, 1H), 3.23 (d, J = 16 Hz, 1H), 3.11 (t, J = 4.0 Hz, 1H) |
| 409 | MD | 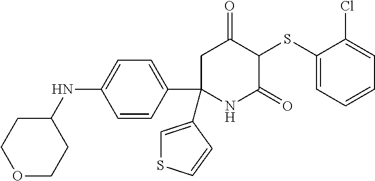 [SMILES: O=C1CC(c2ccc(NC3CCOCC3)cc2)(c2ccsc2)NC(=O)C1Sc1ccccc1Cl] | 3-(2-chlorophenyl)sulfanyl- 6-[4-(tetrahydropyran- 4-ylamino)phenyl]-6- (3-thienyl)piperidine- 2,4-dione was prepared in 3.8% yield according to the example 7, Step H substituting 2-methyl- morpholine for tetrahydro- 2H-pyran-4-amine | ¹H NMR (400 MHz, DMSO-d₆) δ = 8.23 (s, 1H), 7.46 (dd, J = 8Hz, 8 Hz, 1H), 7.24 (s, 1H) 7.19-7.14 (m, 3H), 6.87 (dd, J = 8.0 8.0 Hz, 1H), 6.76 (dd, J = 8.0 8.0 Hz, 1H), 6.67 (d, J = 8.0 Hz, 2H), 6.02 (d, J = 8.0 Hz 1H), 3.98-3.95 (m, 2H), 3.56-3.50 (m, 3H), 3.35 (s, 2H), 2.00-1.97 (m, 2H), 1.50- 1.47 (m, 2H) |
| 410 | MD | 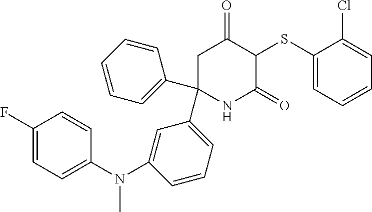 [SMILES: CN(c1ccc(F)cc1)c1cccc(C2(c3ccccc3)CC(=O)C(Sc3ccccc3Cl)C(=O)N2)c1] | 3-(2-chlorophenyl)sulfanyl- 6-[3-(4-fluoro-N-methyl- anilino)phenyl]-6-phenyl- piperidine-2,4-dione | |
| 411 | MD | 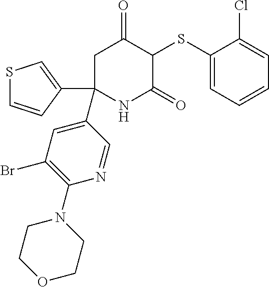 [SMILES: O=C1CC(c2ccsc2)(c2cnc(N3CCOCC3)c(Br)c2)NC(=O)C1Sc1ccccc1Cl] | 6-(5-bromo-6-morpholino- 3-pyridyl)-3-(2-chloro- phenyl)sulfanyl-6-(3- thienyl)piperidine-2,4- dione was prepared in 17% yield according to the Method 1 substututing 6-bromopicolinic acid for 5-bromo-6-morpholino- nicotinic acid | 1H NMR (400 MHz, METHANOL- d4) d = 8.22 (dd, J = 2.4 Hz, 1H), 8.02 (dd, J = 2.0 Hz, 1H), 7.61 (dd, J = 5.2, 3.2 Hz, 1H), 7.41 (dd, J = 2.8, 1.6 Hz, 1H), 7.26 (dd, J = 8.0, 1.6 Hz, 1H), 7.21 (dd, J = 8.0, 8.0 Hz, 1H), 6.98 (dd, J = 8.0, 8.0 Hz, 1H), 6.83 (dd, J = 8.0, 8.0 Hz, 1H), 6.07 (dd, J = 8.0, 1.6 Hz, 1H), 3.86 (t, J = 4.4 Hz, 4H), 3.51 (s, 2H), 3.36 (t, J = 4.4 Hz, 4H) |
| 412 | SS | 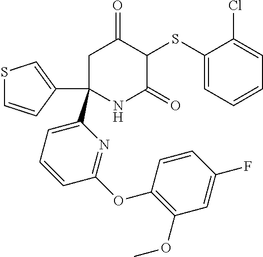 [SMILES: COc1cc(F)ccc1Oc1cccc([C@@]2(c3ccsc3)CC(=O)C(Sc3ccccc3Cl)C(=O)N2)n1] | (6S)-3-(2-chlorophenyl) sulfanyl-6-[6-(4-fluoro- 2-methoxy-phenoxy)- 2-pyridyl]-6-(3-thienyl) piperidine-2,4-dione was prepared as described in example 334. | |
| 413 | SS | 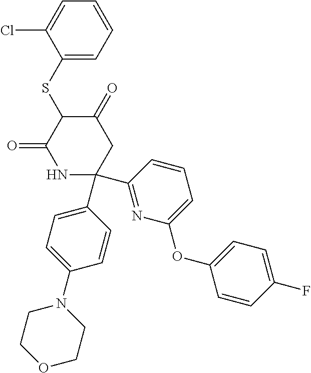 [SMILES: O=C1CC(c2ccc(N3CCOCC3)cc2)(c2cccc(Oc3ccc(F)cc3)n2)NC(=O)C1Sc1ccccc1Cl] | 3-(2-chlorophenyl)sulfanyl- 6-[6-(4-fluorophenoxy)- 2-pyridyl]-6-(4-morpho- linophenyl)piperidine- 2,4-dione | |
| 414 | SS | 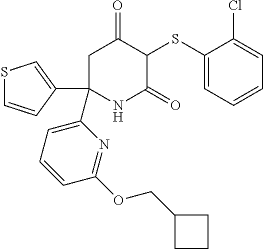 [SMILES: O=C1CC(c2ccsc2)(c2cccc(OCC3CCC3)n2)NC(=O)C1Sc1ccccc1Cl] | 3-(2-chlorophenyl)sulfanyl- 6-(3-thienyl)-6-[6- (4,4,4-trifluorobutoxy)- 2-pyridyl]piperidine- 2,4-dione was prepared in 41% yield according to example 3, Step A substituting propan-2-ol for cyclobutylmethanol | ¹H NMR (400 MHz, DMSO-d₆) δ = 7.67 (dd, J = 8.0, 8.0 Hz, 1H), 7.40 (dd, J = 4.2 Hz, 4.2 Hz, 1H) 7.28 (d, J = 2.8 Hz, 1H), 7.16-7.11 (m, 3H), 6.88 (dd, J = 8.0, 8.0 Hz, 1H), 6.72-6.70 (m, 2H), 6.02 (d, J = 8.0 Hz, 1H), 4.31 (d, J = 8.4 Hz, 1H), 3.74 (d, J = 16H, 1H), 3.41 (d, J = 16 Hz 1H), 2.78-2.70 (m, 1H), 2.08- 2.06 (m, 2H), 1.89-1.86 (m, 4H) |
| 415 | MD | 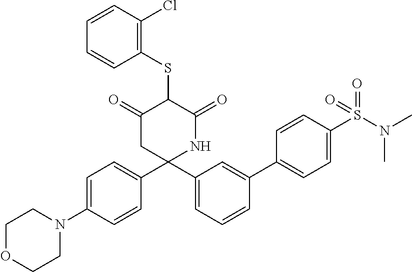 [SMILES: CN(C)S(=O)(=O)c1ccc(-c2cccc(C3(c4ccc(N5CCOCC5)cc4)CC(=O)C(Sc4ccccc4Cl)C(=O)N3)c2)cc1] | 4-[3-[5-(2-chlorophenyl) sulfanyl-2-[4-morpho- linophenyl)-4,6-dioxo-2- piperidyl]phenyl]-N,N- dimethyl-benzene sulfonamide | |
| 416 | MD | 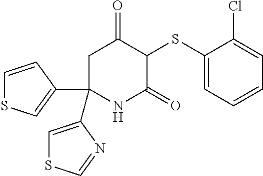 [SMILES: O=C1CC(c2ccsc2)(c2cscn2)NC(=O)C1Sc1ccccc1Cl] | 3-(2-chlorophenyl)sulfanyl- 6-thiazol-4-yl-6-(3- thienyl)piperidine-2,4- dione was prepared in 3% yield according to example 1 substituting 6-bromopicolinic acid for thiazole-4-carboxylic acid. | 1H NMR (400 MHz, METHANOL- d4) δ = 9.05 (d, J = 2.0 Hz, 1H), 7.49-7.47 (m, 1H), 7.45 (d, J = 2.0 Hz, 1H), 7.28 (dd, J = 5.2, 1.2 Hz, 1H), 7.24-7.20 (m, 2H), 6.98 (dd, J = 5.2, 5.2 Hz, 1H), 6.88 (dd, J = 5.2, 5.2 Hz, 1H), 6.14 (dd, J = 6.8, 1.2 Hz, 1H), 3.82 (d, J = 16.8 Hz, 1H), 3.52 (d, J = 16.8 Hz, 1H) |

*ST: Stereochemistry: SS = Single Stereoisomer; MD = Mixture of Diastereoisomers

### Example 417

### 3-(2-chlorophenyl)sulfanyl-6-[6-(4-fluoroanilino)-2-pyridyl]-6-(3-thienyl)piperidine-2,4-dione (racemate)

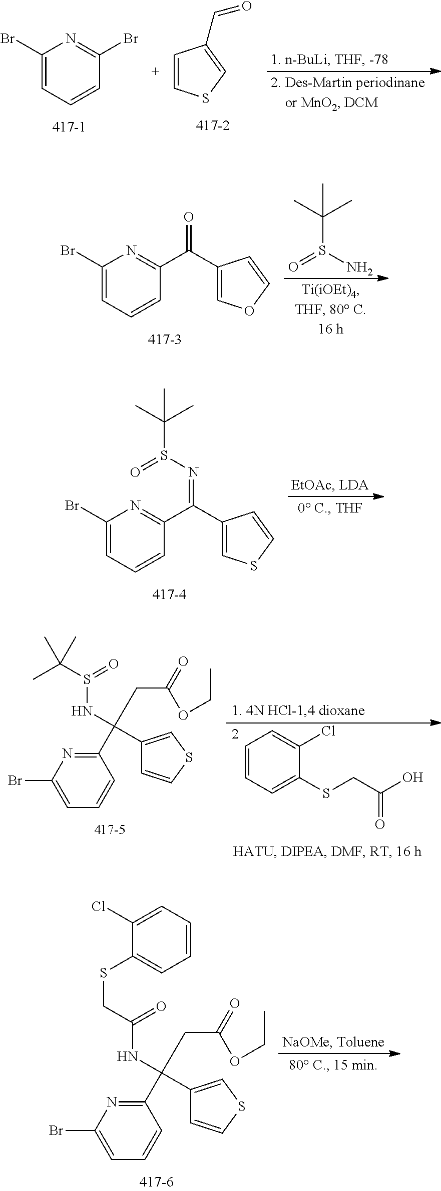

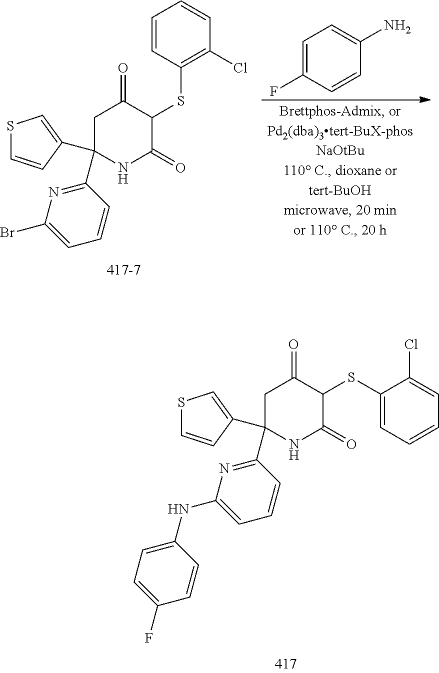

To a solution of 2,6-dibromopyridine (5.0 g, 21 mmol) in dry THF (50 mL) cooled in dry-ice acetone bath was added 2.5 M butyl lithium (8.4 mL, 21 mmol) and the resulting mixture was stirred for 10 min. To this solution was added thiophene-3-carbaldehyde (2.4 g, 21 mmol) and the resulting mixture was stirred at −78 for 10 min. The reaction mixture was quenched with water and allowed to warm to room temperature and extracted with ethyl acetate. The organic layer was washed with brine, dried over sodium sulfate and concentrated. The residue was purified by flash chromatography (silica gel, 0-50% EtOAc/heptane) to obtain (6-bromo-2-pyridin-2-yl)-thiphen-3-yl-methanol (2.6 g, 46%). MS (ESI): m+H=272. 1H NMR (400 MHz, Chloroform-d) δ 7.51 (t, J=7.7 Hz, 1H), 7.40 (d, J=7.8 Hz, 1H), 7.29 (dd, J=5.0, 3.0 Hz, 1H), 7.05-7.01 (m, 1H), 7.26 (s, 1H), 7.20 (d, J=7.6 Hz, 1H), 7.03 (dd, J=5.0, 1.2 Hz, 1H), 5.84 (d, J=4.0 Hz, 1H), 4.17 (d, J=4.9 Hz, 1H).

A mixture of (6-bromo-2-pyridin-2-yl)-thiphen-3-yl-methanol (2.6 g, 9.6 mmol) and des martin periodinane (6.3 g, 14 mmol) in DCM (50 mL) was stirred at ambient temperature for 2 h. The solids were removed by filtration through celite and washed well with ethyl acetate. The organic layer washed with aqueous sodium bicarbonate, water, brine and dried over sodium sulfate and concentrated. The residue was purified by flash chromatography (silica gel, 0-50% EtOAc/heptane) to obtain (6-bromo-pyridin-2-yl)-thiophen-3-yl-methanone (1.8 g, 70%). MS (ESI): m+H=270; ¹H NMR (400 MHz, Chloroform-d) δ 8.92 (dd, J=3.0, 1.1 Hz, 1H), 8.14-8.08 (m, 1H), 7.88 (dd, J=5.1, 1.2 Hz, 1H), 7.75 (t, J=7.7 Hz, 1H), 7.67 (d, J=7.8 Hz, 1H), 7.34 (dd, J=5.1, 3.0 Hz, 1H).

A mixture of (6-bromo-pyridin-2-yl)-thiophen-3-yl-methanone (2.5 g, 9.3 mmol), 2-Methyl-propane-2-sulfinic acid amide (1.7 g, 14 mmol) and titanium tetraethoxide (4.3 g, 19 mmol) in THF was heated at reflux for 20 h. The reaction mixture was cooled, diluted with water and stirred over ethyl acetate. The solids were removed by filtration through celite. Organic layer separated, washed with brine, dried over sodium sulfate and concentrated. The residue was purified by flash chromatography (silica gel 0-100% EtOAc/heptane) to afford the 2-Methyl-propane-2-sulfinic acid 1-(6-bromo-pyridin-2-yl)-1-thiophen-3-yl-meth-(Z)-ylideneamide (2.1 g, 61%). MS (ESI): m+H=373.

To a solution of ethyl acetate in dry THF (0.47 g, 5.4 mmol) cooled in dry-ice-acetone bath was added 2M LDA in heptane/ethylbenzene (2.7 mL, 5.4 mmol) and the mixture was stirred for 10 min and a solution of 2-methyl-propane-2-sulfinic acid 1-(6-bromo-pyridin-2-yl)-1-thiophen-3-yl-meth-(Z)-ylideneamide (1.0 g, 2.7 mmol) in THF (3 mL) was added slowly. The resulting mixture was stirred for 10 min, quenched with saturated ammonium chloride, allowed to warm to ambient temperature and extracted with ethyl acetate. The organic layer separated, washed with brine, dried over sodium sulfate and concentrated. The was purified by flash chromatography (silica gel 0-100% EtOAc/heptane) to afford 3-(6-bromo-pyridin-2-yl)-3-(2-methyl-propane-2-sulfinylamino)-3-thiophen-3-yl-propionic acid ethyl ester (1.1 g,). MS (ESI): m+H=461.

3-(6-Bromo-pyridin-2-yl)-3-(2-methyl-propane-2-sulfinylamino)-3-thiophen-3-yl-propionic acid ethyl ester (1.1 g, 2.0 mmol) was dissolved in DCM (5 mL) and 4N HCl-1,4-dioxane (4 mL, 16 mmol) was added and the mixture stirred for 15 min. The solvents were removed and to the residue were added (2-Chloro-phenylsulfanyl)-acetic acid (0.52 g, 2.6 mmol) HATU (1.1 g, 2.8 mmol) and DMF (5 mL) followed by DIPEA (1.4 mL, 7.8 mmol). The resulting mixture was stirred 1 h and then diluted with water and extracted with ethyl acetate. The organic layer was washed with brine several times, dried over sodium sulfate and concentrated. The residue was dissolved in toluene (5 ml) and 25% sodium methoxide in methanol (1.5 mL, 6.5 mmol) was added and the resulting dark solution heated at 80° C. for 15 min. The reaction mixture was cooled, acidified with 1N HCl and extracted with ethyl acetate. The organic layer washed with brine, dried over sodium sulfate and concentrated. Purification of the residue by column chromatography (silica gel, 0-100% EtOAc/heptane) afforded 6-(6-bromo-2-pyridyl)-3-(2-chlorophenyl)sulfanyl-6-(3-thienyl)piperidine-2,4-dione (0.56 g). MS (ESI): m+H=566. 1H NMR (400 MHz, DMSO-d6) δ 11.62 (s, 1H), 8.68 (s, 1H), 7.73-7.61 (m, 2H), 7.61-7.53 (m, 2H), 7.43-7.33 (m, 4H), 7.30 (dd, J=8.0, 1.3 Hz, 1H), 7.03-6.93 (m, 1H), 6.77-6.69 (m, 1H), 5.84 (dd, J=8.0, 1.5 Hz, 1H), 3.49 (s, 2H).

A mixture of 6-(6-bromo-2-pyridyl)-3-(2-chlorophenyl)sulfanyl-6-(3-thienyl)piperidine-2,4-dione (0.50 g, 1 mmol), 4-fluoroaniline (0.22 g, 2 mmol) and Brettphos-Admix (0.13 g, 0.1 mmol) and sodium tert-butoxide (0.3 g, 3 mmol) in a mixture of tert-butanol and 1,4-dioxane (1:1 mixture, 10 ml) was heated 105° C. in a sealed tube for 1 h. The reaction mixture was cooled and the solid collected by filtration. The solid was acidified with 1N HCl and then dissolved in ethyl acetate. The ethyl acetate layer washed with brine, dried over sodium sulfate and concentrated. Purification by column chromatography (silica gel, 20-100% EtOAc/heptane) afforded 3-(2-chlorophenyl)sulfanyl-6-[6-(4-fluoroanilino)-2-pyridyl]-6-(3-thienyl)piperidine-2,4-dione (racemate) (0.30 g, 56%). MS(ESI): m+H=524; 1H NMR (400 MHz, DMSO-d6) δ 11.48 (s, 1H), 9.12 (s, 1H), 8.37 (s, 1H), 7.66-7.57 (m, 3H), 7.53 (dd, J=5.1, 3.0 Hz, 1H), 7.39 (dd, J=3.0, 1.4 Hz, 1H), 7.30 (dd, J=7.9, 1.3 Hz, 1H), 7.18 (dd, J=5.1, 1.4 Hz, 1H), 7.11-7.03 (m, 3H), 6.99-6.92 (m, 1H), 6.81 (ddd, J=8.5, 7.3, 1.3 Hz, 1H), 6.72 (dd, J=8.3, 0.7 Hz, 1H), 6.06 (dd, J=8.0, 1.5 Hz, 1H), 3.77 (d, J=16.4 Hz, 1H), 3.45 (d, J=16.5 Hz, 1H).

Enantiomer 1: Chiral SFC (column: AS, EtOH w/0.1% FA): RT=0.892 min.; 1H NMR (400 MHz, DMSO-d6) δ 11.49 (s, 1H), 9.12 (s, 1H), 7.67-7.57 (m, 3H), 7.52 (dd, J=5.1, 2.9 Hz, 1H), 7.39 (dd, J=3.0, 1.4 Hz, 1H), 7.30 (dd, J=7.9, 1.3 Hz, 1H), 7.18 (dd, J=5.1, 1.4 Hz, 1H), 7.12-7.00 (m, 3H), 6.99-6.92 (m, 1H), 6.85-6.78 (m, 1H), 6.72 (d, J=8.3 Hz, 1H), 6.06 (dd, J=8.0, 1.5 Hz, 1H), 3.76 (d, J=16.5 Hz, 1H), 3.44 (d, J=16.5 Hz, 1H).

Enantiomer 2: Chiral SFC (column: AS, EtOH w/0.1% FA): RT=1.276 min. 1H NMR (400 MHz, DMSO-d6) δ 11.48 (s, 1H), 9.12 (s, 1H), 8.36 (s, 1H), 7.67-7.55 (m, 3H), 7.53 (dd, J=5.0, 3.0 Hz, 1H), 7.39 (dd, J=3.0, 1.4 Hz, 1H), 7.30 (dd, J=7.9, 1.3 Hz, 1H), 7.18 (dd, J=5.1, 1.4 Hz, 1H), 7.11-7.02 (m, 3H), 7.00-6.93 (m, 1H), 6.86-6.78 (m, 1H), 6.72 (d, J=8.3 Hz, 1H), 6.06 (dd, J=7.9, 1.5 Hz, 1H), 3.77 (d, J=16.4 Hz, 1H), 3.45 (d, J=16.4 Hz, 1H).

### Example 418

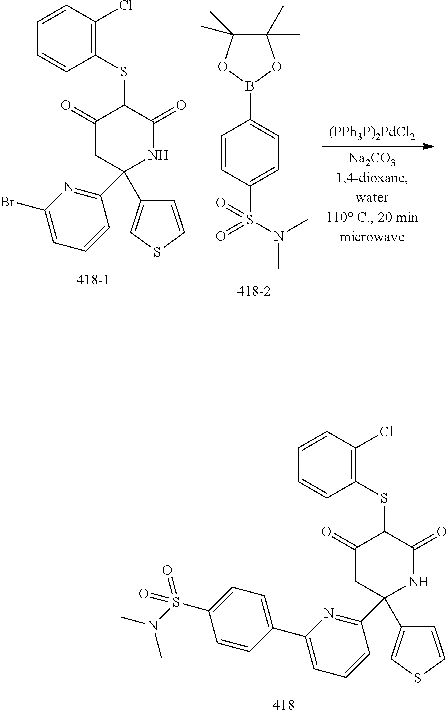

4-[6-[5-(2-chlorophenyl)sulfanyl-4,6-dioxo-2-(3-thienyl)-2-piperidyl]-2-pyridyl]-N,N-dimethyl-benzenesulfonamide (racemate)

A mixture of 6-(6-bromo-2-pyridyl)-3-(2-chlorophenyl)sulfanyl-6-(3-thienyl)piperidine-2,4-dione (0.50 g, 1 mmol), 4-fluoroanili (0.06 g, 0.10 mmol), N,N-dimethyl-4-(4,4,5,5-tetramethyl-1,3,2-dioxaborolan-2-yl)benzenesulfonamide (0.05 g, 0.12 mmol), PdCl₂(PPh₃)₂(0.015 g, 0.014 mmol) and sodium carbonate (0.05 g, 0.47 mmol) in 1,4-dioxane was heated at 110° C. for 20 min in a microwave reactor. The reaction mixture was cooled, acidified by with 1N HCl and extracted with ethyl acetate. The organic layer was washed with brine, dried over sodium sulfate and concentrated. Purification by column chromatography (silica gel, 0-100% EtOAc/heptane) afforded 4-[6-[5-(2-chlorophenyl)sulfanyl-4,6-dioxo-2-(3-thienyl)-2-piperidyl]-2-pyridyl]-N,N-dimethyl-benzenesulfonamide (0.04 g,): MS(ESI): m+H=598; 1H NMR (400 MHz, DMSO-d6) δ 11.69 (s, 1H), 8.65 (s, 1H), 8.51-8.43 (m, 2H), 8.16-8.03 (m, 2H), 7.88-7.81 (m, 2H), 7.74 (d, J=7.7 Hz, 1H), 7.54 (dd, J=5.1, 3.0 Hz, 1H), 7.38 (dd, J=3.0, 1.4 Hz, 1H), 7.26 (dd, J=7.9, 1.2 Hz, 1H), 7.19 (dd, J=5.0, 1.3 Hz, 1H), 6.89 (td, J=7.7, 1.5 Hz, 1H), 6.56 (td, J=7.7, 1.3 Hz, 1H), 5.81 (dd, J=8.0, 1.5 Hz, 1H), 4.14 (d, J=16.3 Hz, 1H), 3.45 (d, J=16.2 Hz, 1H), 2.65 (s, 6H).

### Example 419

### 3-(2-chlorophenyl)sulfanyl-6-[6-(4-fluorophenoxy)-2-pyridyl]-6-(3-thienyl)piperidine-2,4-dione

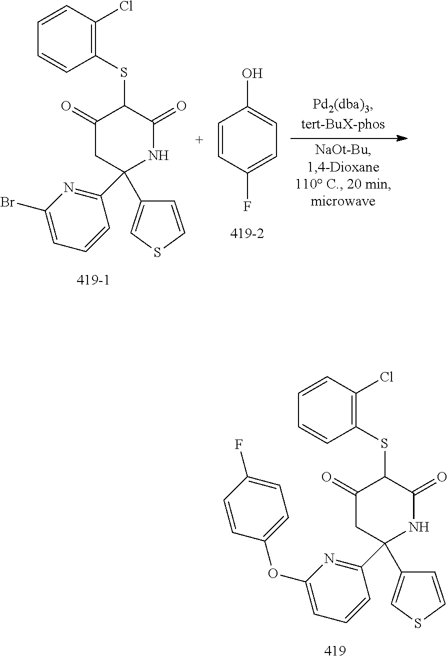

A mixture 6-(6-bromopyridin-2-yl)-3-((2-chlorophenyl)thio)-6-(thiophen-3-yl)piperidine-2,4-dione (0.50 g, 1 mmol), 4-fluorophenol (0.34 g, 3 mmol), Pd₂(dba)₃ (0.10 g, 0.1 mmol), (0.10 g, (0.10 g, 0.24 mmol) and sodium tert-butoxide (0.3 g, 3 mmol) in 1,4-dioxane (10 ml) was heated at 110° C. for 30 min in the microwave reactor. The reaction mixture was cooled and the solid collected by filtration. The solid was acidified with 1N HCl and then dissolved in ethyl acetate. The ethyl acetate layer washed with brine, dried over sodium sulfate and concentrated. Purification by column chromatography (silica gel, 20-100% EtOAc/heptane) afforded 3-(2-chlorophenyl)sulfanyl-6-[6-(4-fluorophenoxy)-2-pyridyl]-6-(3-thienyl)piperidine-2,4-dione racemate (0.30 g, 56%). LC/MS: m+H=525. 1H NMR (400 MHz, DMSO-d6) δ 11.44 (s, 1H), 8.43 (s, 1H), 7.98-7.87 (m, 1H), 7.50 (dd, J=5.1, 3.0 Hz, 1H), 7.41 (d, J=7.5 Hz, 1H), 7.32-7.22 (m, 3H), 7.16-7.08 (m, 2H), 7.08-6.91 (m, 3H), 6.84-6.75 (m, 1H), 5.95 (dd, J=8.0, 1.5 Hz, 1H), 3.57 (d, J=16.5 Hz, 1H), 3.25 (s, 1H).

Enantiomer 1: Chiral SFC (Column: AD; MeOH/0.1% NH₄OH): RT=0.521

Enantiomer 2: Chiral SFC (Column: AD; MeOH/0.1% NH₄OH): RT=0.775

### Example 420

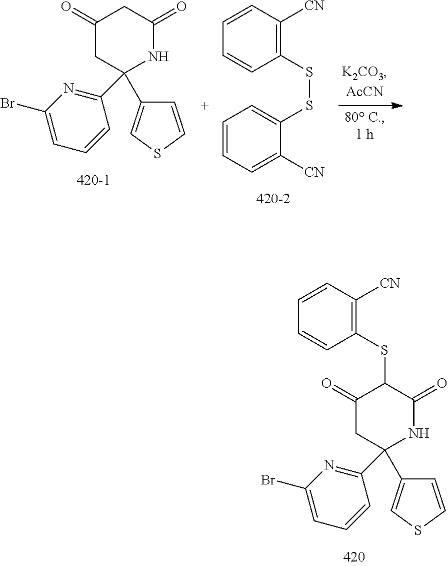

### 2-[[6-(6-bromo-2-pyridyl)-2,4-dioxo-6-(3-thienyl)-3-piperidyl]sulfanyl]benzonitrile (MD)

A mixture of 6-(6-bromo-2-pyridyl)-6-(3-thienyl)piperidine-2,4-dione (0.14 g, 0.40 mmol), 2-[(2-cyanophenyl)disulfanyl]benzonitrile (0.21 g, 0.80 mmol) and potassium carbonate (0.17 g, 1.20 mmol) was heated in acetonitrile (5 mL) for 1 h. The reaction mixture was cooled, acidified with dil HCl and extracted with ethyl acetate. The organic layer was washed with brine, dried over sodium sulfate and concentrated. Purification of the residue by column chromatography (silica gel, 0-100% EtOAc/heptane) afforded 2-[[6-(6-bromo-2-pyridyl)-2,4-dioxo-6-(3-thienyl)-3-piperidyl]sulfanyl]benzonitrile (0.04 g, 20%): MS (ESI): m+H=484; 1H NMR (400 MHz, DMSO-d6) δ 11.94 (s, 1H), 8.58 (s, 1H), 7.86 (t, J=7.8 Hz, 1H), 7.72-7.62 (m, 3H), 7.56 (dd, J=5.1, 3.0 Hz, 1H), 7.37-7.30 (m, 1H), 7.15 (td, J=5.2, 4.6, 1.7 Hz, 3H), 6.16-6.00 (m, 1H), 3.84 (d, J=16.4 Hz, 1H), 3.43 (d, J=16.4 Hz, 1H).

### Example 421

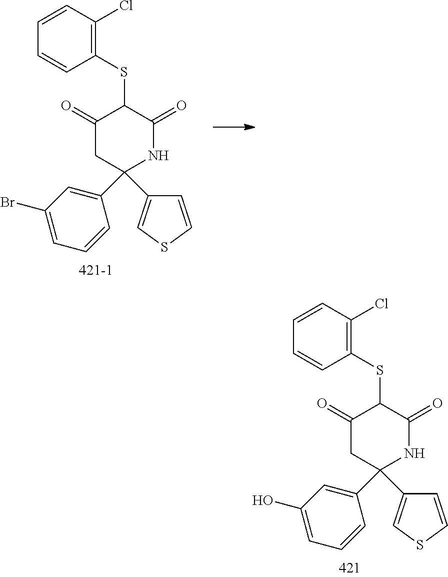 [SMILES: O=C1CC(c2ccsc2)(c2cccc(Br)c2)NC(=O)C1Sc1ccccc1Cl ; O=C1CC(c2ccsc2)(c2cccc(O)c2)NC(=O)C1Sc1ccccc1Cl]

A mixture of 6-(3-bromophenyl)-3-(2-chlorophenyl)sulfanyl-6-(3-thienyl)piperidine-2,4-dione (0.50 g, 1 mmol), 4-fluorophenol (0.34 g, 3 mmol), Pd₂(dba)₃ (0.10 g, 0.1 mmol), (0.10 g, (0.10 g, 0.24 mmol) and sodium tert-butoxide (0.3 g, 3 mmol) in 1,4-dioxane (10 ml) was heated at 110° C. for 30 min. The reaction mixture was cooled, acidified by with 1N HCl and extracted with ethyl acetate. The organic layer was washed with brine, dried over sodium sulfate and concentrated. Purification by column chromatography (silica gel, 0-100% EtOAc/heptane) afforded 3-(2-chlorophenyl)sulfanyl-6-(3-hydroxyphenyl)-6-(3-thienyl)piperidine-2,4-dione (25 mg): MS (ESI): m+H=430; DMSO-d6) δ 11.44 (s, 1H), 9.43 (s, 1H), 8.33 (s, 1H), 7.56 (dd, J=5.1, 2.9 Hz, 1H), 7.35 (dd, J=3.0, 1.4 Hz, 1H), 7.28 (dd, J=8.0, 1.3 Hz, 1H), 7.20-7.13 (m, 2H), 7.02-6.92 (m, 1H), 6.87-6.67 (m, 4H), 5.94 (dd, J=8.0, 1.5 Hz, 1H), 3.44-3.36 (m, 2H).

### Example 422

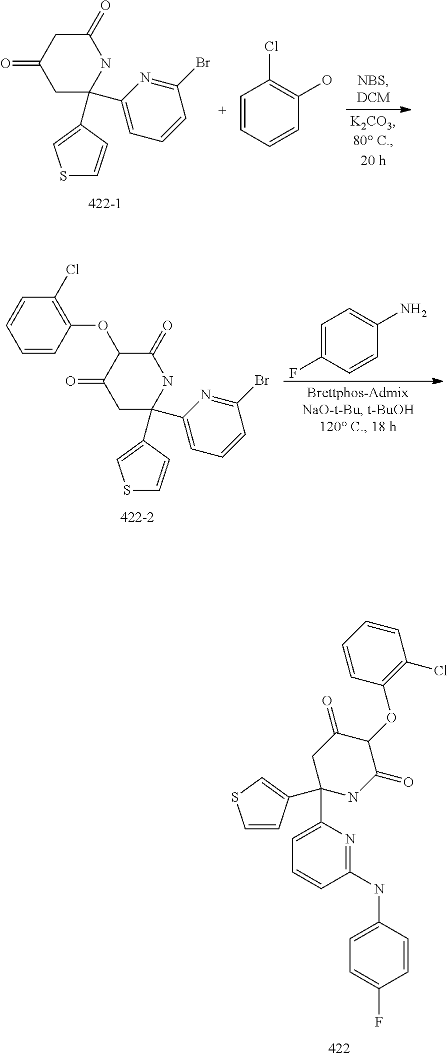

To a solution of 1-7 (0.21 g, 0.60 mmol) in DCM (10 mL) was added NBS (0.10 g, 0.60 mmol) and the resulting mixture was stirred for 1 h. The reaction mixture was washed with water, brine, dried over sodium sulfate and concentrated. The resulting residue was dissolved in acetonitrile (5 ml) and 2-chlorophenol (0.15 g, 1.2 mmol) and potassium carbonate (0.16 g, 1.2 mmol) were added and the mixture heated at 85° C. for 20 h. The reaction mixture was cooled, acidified with dil.HCl and extracted with ethyl acetate. Organic layer washed with brine, dried over sodium sulfate and concentrated. Purification by column chromatography (silica gel, 0-100% EtOAc/heptane) afforded 6-(6-bromo-2-pyridyl)-3-(2-chlorophenoxy)-6-(3-thienyl)piperidine-2,4-dione (0.20 g, 70%): MS (ESI): m+H=479; 1H NMR (400 MHz, DMSO-d6) δ 10.74 (s, 1H), 8.33 (s, 1H), 7.89-7.81 (m, 1H), 7.70-7.61 (m, 2H), 7.58-7.51 (m, 1H), 7.43-7.29 (m, 2H), 7.15 (dd, J=5.1, 1.4 Hz, 1H), 6.99-6.83 (m, 2H), 6.13 (dd, J=8.2, 1.5 Hz, 1H), 3.74-3.66 (m, 1H), 3.36 (d, J=16.2 Hz, 1H). 6-(6-bromo-2-pyridyl)-3-(2-chlorophenoxy)-6-(3-thienyl)piperidine-2,4-dione (0.05 g, 0.10 mmol) was converted to 3-(2-chlorophenoxy)-6-[6-(4-fluoroanilino)-2-pyridyl]-6-(3-thienyl)piperidine-2,4-dione (0.42 mg, 80%) as described previously: MS(ESI): m+H=508. 1H NMR (400 MHz, DMSO-d6) δ 9.10 (s, 1H), 8.04 (s, 1H), 7.66-7.57 (m, 3H), 10.87-10.41 (m, 1H), 7.49 (dd, J=5.1, 3.0 Hz, 1H), 7.40 (dd, J=3.0, 1.4 Hz, 1H), 7.34 (dd, J=7.8, 1.7 Hz, 1H), 7.17 (dd, J=5.1, 1.4 Hz, 1H), 7.10-6.97 (m, 3H), 6.97-6.83 (m, 2H), 6.70 (d, J=8.2 Hz, 1H), 6.17 (dd, J=8.2, 1.6 Hz, 1H), 3.63 (d, J=16.2 Hz, 1H), 3.36 (s, 1H).

### Example 423

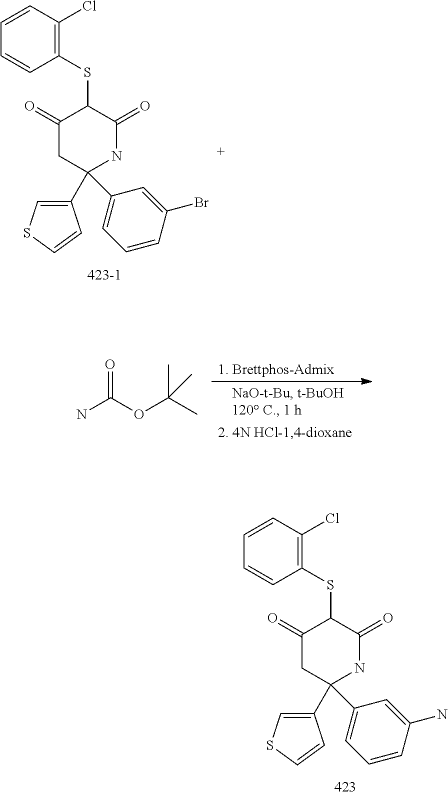

A mixture of 6-(3-bromophenyl)-3-(2-chlorophenyl)sulfanyl-6-(3-thienyl)piperidine-2,4-dione (0.07 g, 0.14 mmol), tert-butyl carbamate (0.05 g, 0.43 mmol), Brettphos-Admix (0.02 g, 0.02 mmol) and sodium tert-butoxide (0.04 mg, 0.43 mmol) in tert-butanol was heated at 120° C. for 1 h. The reaction mixture was cooled, acidified with dil HCl and extracted with ethyl acetate. The organic layer was washed with brine dried over sodium sulfate and concentrated. The residue was dissolved in DCM (2 mL) and 4N-HCl-1,4-dioxane was added and stirred for 30 min. The reaction mixture was concentrated, treated with sodium bicarbonate and extracted with ethyl acetate. The organic layer was washed with brine, dried over sodium sulfate. Purification by column chromatography (silica gel, 0-100% iPrAc/heptane) afforded 6-(3-aminophenyl)-3-(2-chlorophenyl)sulfanyl-6-(3-thienyl)piperidine-2,4-dione (0.02 g, 32%)>MS(ESI): m+H=429; 1H NMR (400 MHz, DMSO-d6) δ 8.29 (s, 1H), 7.56 (dd, J=5.0, 3.0 Hz, 1H), 7.34 (dd, J=2.9, 1.4 Hz, 1H), 7.29 (dd, J=7.9, 1.3 Hz, 1H), 7.16 (dd, J=5.1, 1.4 Hz, 1H), 7.05-6.95 (m, 2H), 6.85-6.75 (m, 1H), 6.61 (t, J=2.0 Hz, 1H), 6.56-6.48 (m, 2H), 5.96 (dd, J=7.9, 1.5 Hz, 1H), 3.42-3.33 (m, 2H).

### Example 424

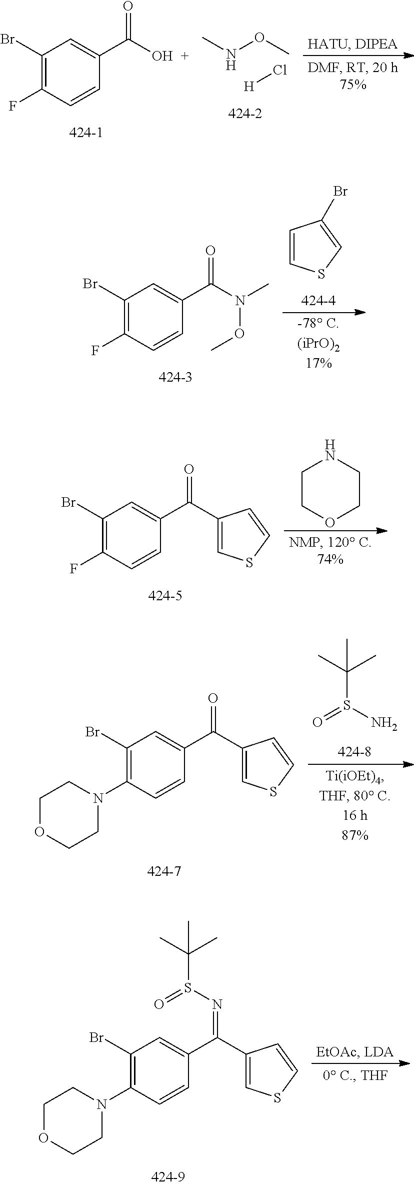

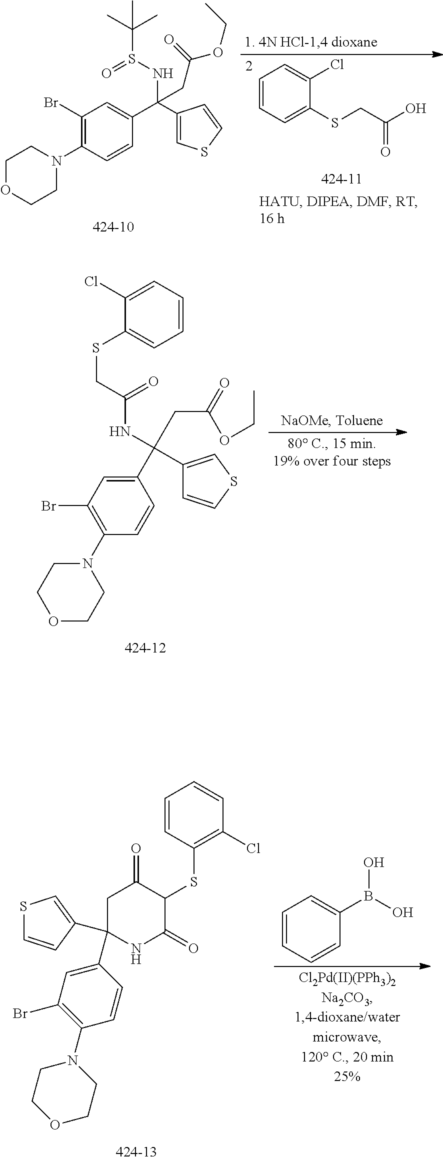

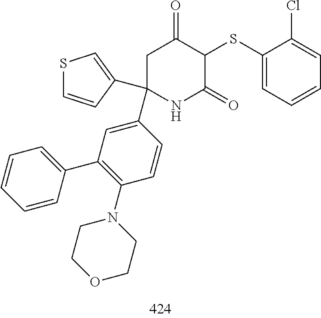 [SMILES: O=C1CC(c2ccsc2)(c2ccc(N3CCOCC3)c(-c3ccccc3)c2)NC(=O)C1Sc1ccccc1Cl]

All steps and conditions are described in examples hereinabove.

### Example 425

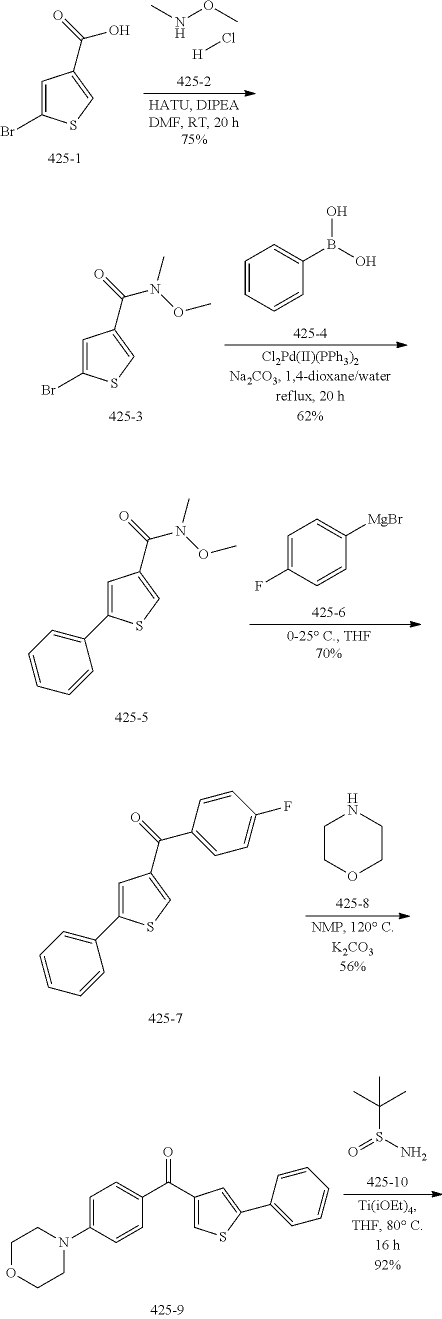

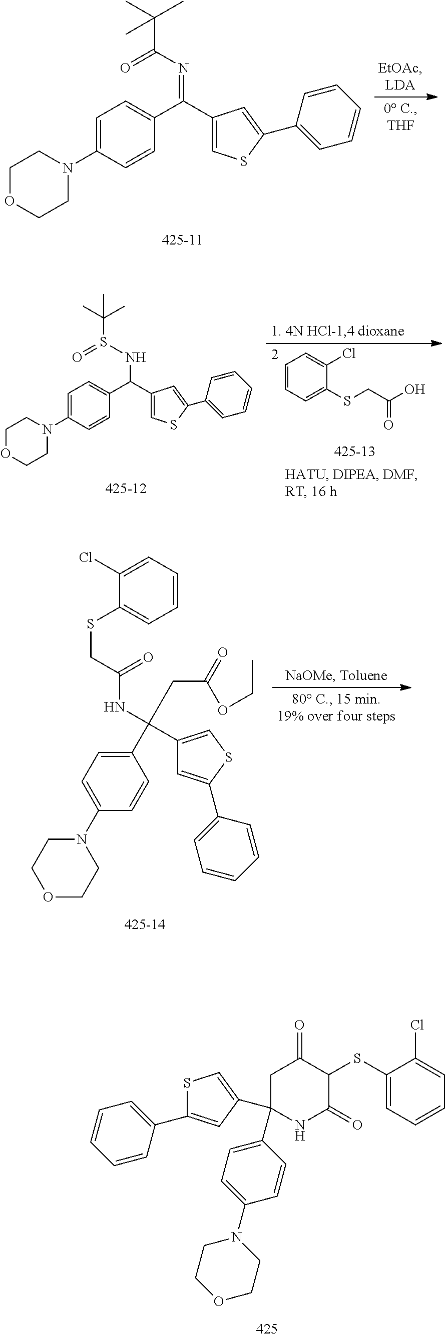

All steps and conditions are described in examples hereinabove.

| Ex. | | | IUPAC | Characterization data |
|---|---|---|---|---|
| No. | ST* | Structure | NAME/synthesis | (NMR or MS) |
| 426 | MD | 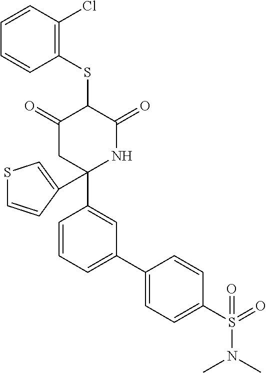 [SMILES: CN(C)S(=O)(=O)c1ccc(-c2cccc(C3(c4ccsc4)CC(=O)C(Sc4ccccc4Cl)C(=O)N3)c2)cc1] | 4-[3-[5-(2- chlorophenyl) sulfanyl-4,6- dioxo-2-(3- thienyl)-2- piperidyl] phenyl]-N,N- dimethyl- benzene- sulfonamide | H NMR (400 MHz, DMSO-d6) δ 11.52 (s, 1H), 8.62 (s, 1H), 7.98- 7.91 (m, 3H), 7.87-7.80 (m, 2H), 7.73 (d, J = 7.9 Hz, 1H), 7.63- 7.50 (m, 2H), 7.48-7.41 (m, 2H), 7.31-7.21 (m, 2H), 6.97-6.89 (m, 1H), 6.66 (t, J = 7.5 Hz, 1H), 5.88 (dd, J = 8.0, 1.4 Hz, 1H), 3.64-3.48 (m, 2H), 2.64 (s, 6H) |
| 427 | MD | 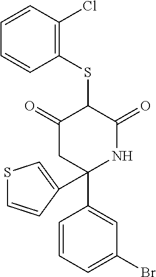 [SMILES: O=C1CC(c2ccsc2)(c2cccc(Br)c2)NC(=O)C1Sc1ccccc1Cl] | 6-(3-bromo- phenyl)-3-(2- chlorophenyl) sulfanyl-6-(3- thienyl) piperidine-2,4- dione (synthesized as in example 1 starting with 1,3-dibromo- benzene) | δ1H NMR (400 MHz, DMSO- d6) δ 11.85-11.00 (m, 1H), 8.53 (s, 1H), 7.65 (t, J = 1.9 Hz, 1H), 7.64-7.53 (m, 2H), 7.45-7.25 (m, 5H), 7.18 (dd, J = 5.1, 1.4 Hz, 1H), 7.04- 6.94 (m, 1H), 6.77 (td, J = 7.7, 1.3 Hz, 1H), 5.89 (dd, J = 8.0, 1.4 Hz, 1H), 3.46 (d, J = 4.3 Hz, 2H), 11.85-11.00 (m, 1H) |
| 428 | MD | 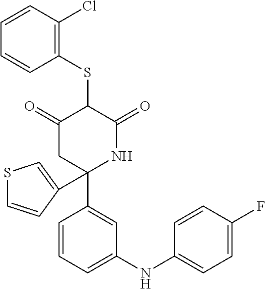 [SMILES: O=C1CC(c2ccsc2)(c2cccc(Nc3ccc(F)cc3)c2)NC(=O)C1Sc1ccccc1Cl] | 3-(2-chloro- phenyl) sulfanyl-6- [3-(4-fluoro anilino) phenyl]-6- (3-thienyl) piperidine- 2,4-dione Synthesized as in ex 1 | 1H NMR (400 MHz, DMSO- d6) δ 8.30 (s, 1H), 8.16 (s, 1H), 7.58 (dd, J = 5.1, 2.9 Hz, 1H), 7.37 (dd, J = 2.9, 1.4 Hz, 1H), 11.95-10.89 (m, 1H), 7.28 (dd, J = 7.8, 1.3 Hz, 1H), 7.25-7.16 (m, 2H), 7.07 (t, J = 2.1 Hz, 1H), 7.02 (d, J = 6.7 Hz, 4H), 6.97- 6.91 (m, 2H), 6.86-6.81 (m, 1H), 6.80-6.76 (m, 1H), 5.98 (dd, J = 8.1, 1.5 Hz, 1H), 3.43 (m, 2H) |
| 429 | MD | 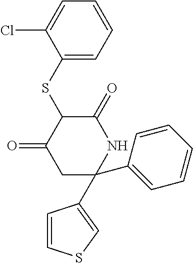 [SMILES: O=C1CC(c2ccccc2)(c2ccsc2)NC(=O)C1Sc1ccccc1Cl] | 3-(2-chloro- phenyl) sulfanyl-6- phenyl-6- (3-thienyl) piperidine- 2,4-dione Synthesized as in ex 1 | 1H NMR (400 MHz, DMSO- d6) δ 11.46 (s, 1H), 8.48 (s, 1H), 7.58 (dd, J = 5.1, 2.9 Hz, 1H), 7.46-7.26 (m, 7H), 7.17 (dd, J = 5.1, 1.4 Hz, 1H), 6.96 (td, J = 7.6, 1.5 Hz, 1H), 6.81- 6.67 (m, 1H), 5.86 (dd, J = 8.0, 1.4 Hz, 1H), 3.44 (s, 2H). |
| 430 | MD | 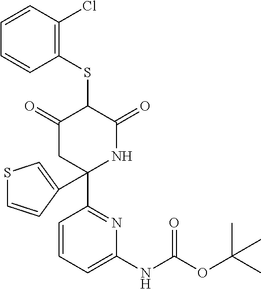 [SMILES: CC(C)(C)OC(=O)Nc1cccc(C2(c3ccsc3)CC(=O)C(Sc3ccccc3Cl)C(=O)N2)n1] | tert-butyl N-[6-[5-(2- chlorophenyl) sulfanyl-4,6- dioxo-2-(3- thienyl)-2- piperidyl]-2- pyridyl] carbamate | DMSO-d6) δ 11.40 (s, 1H), 9.63 (s, 1H), 8.33 (s, 1H), 7.80 (t, J = 7.9 Hz, 1H), 7.69 (d, J = 8.2 Hz, 1H), 7.52 (dd, J = 5.1, 3.0 Hz, 1H), 7.43- 7.36 (m, 1H), 7.30 (d, J = 7.8 Hz, 1H), 7.25-7.16 (m, 2H), 6.98 (t, J = 7.6 Hz, 1H), 6.80 (t, J = 7.7 Hz, 1H), 5.97 (dd, J = 8.0, 1.5 Hz, 1H), 3.77 (d, J = 16.4 Hz, 1H), 3.45 (d, J = 16.3 Hz, 1H), 1.48 (s, 9H) |
| 431 | MD | 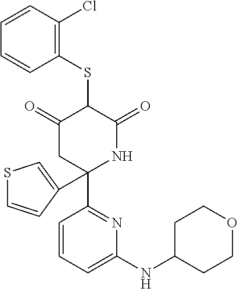 [SMILES: O=C1CC(c2ccsc2)(c2cccc(NC3CCOCC3)n2)NC(=O)C1Sc1ccccc1Cl] | 3-(2-chloro- phenyl) sulfanyl-6- [6-(tetrahydro- pyran-4- ylamino)-2- pyridyl]-6- (3-thienyl) piperidine- 2,4-dione was prepared according to example 417 | 1H NMR (400 MHz, DMSO- d6) δ 11.39 (s, 1H), 8.25 (s, 1H), 7.49 (dd, J = 5.1, 3.0 Hz, 1H), 7.39 (t, J = 7.8 Hz, 1H), 7.29 (d, J = 7.8 Hz, 1H), 7.16 (d, J = 5.1 Hz, 1H), 6.96 (t, J = 7.6 Hz, 1H), 6.78 (t, J = 7.5 Hz, 1H), 6.71 (d, J = 7.3 Hz, 1H), 6.55 (d, J = 7.4 Hz, 1H), 6.40 (d, J = 8.2 Hz, 1H), 6.06-5.95 (m, 1H), 3.82 (t, J = 12.2 Hz, 4H), 3.43 (dt, J = 11.9, 9.3 Hz, 2H), 1.84 (d, J = 12.8 Hz, 2H), 1.45-1.26 (m, 2H) |
| 432 | MD | 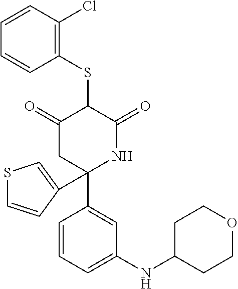 [SMILES: O=C1CC(c2ccsc2)(c2cccc(NC3CCOCC3)c2)NC(=O)C1Sc1ccccc1Cl] | 3-(2-chloro- phenyl) sulfanyl-6- [3-(tetrahydro- pyran-4- ylamino) phenyl]-6- (3-thienyl) piperidine- 2,4-dione was prepared according to example 417 | 1H NMR (400 MHz, DMSO- d6) δ 11.39 (s, 1H), 8.30 (s, 1H), 7.56 (dd, J = 5.1, 3.0 Hz, 1H), 7.39-7.34 (m, 1H), 7.28 (d, J = 7.8 Hz, 1H), 7.21-7.15 (m, 1H), 7.00 (dt, J = 33.7, 7.6 Hz, 2H), 6.76 (t, J = 7.7 Hz, 1H), 6.63 (d, J = 2.0 Hz, 1H), 6.59-6.49 (m, 2H), 5.93 (d, J = 7.8 Hz, 1H), 5.55 (d, J = 8.1 Hz, 1H), 3.83 (dd, J = 10.5, 5.1 Hz, 2H), 3.44-3.33 (m, 5H), 1.84 (d, J = 15.3 Hz, 2H), 1.34 (dd, J = 11.8, 6.4 Hz, 2H). |
| 433 | SS | 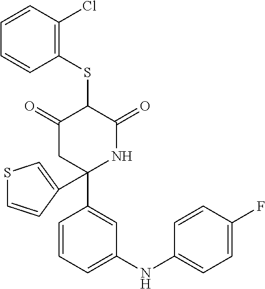 [SMILES: O=C1CC(c2ccsc2)(c2cccc(Nc3ccc(F)cc3)c2)NC(=O)C1Sc1ccccc1Cl] | 3-(2-chloro- phenyl) sulfanyl-6- [3-(4-fluoro anilino) phenyl]-6- (3-thienyl) piperidine- 2,4-dione was prepared according to example 417 | M + H = 523 |
| 434 | SS | 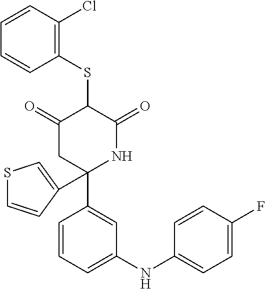 [SMILES: O=C1CC(c2ccsc2)(c2cccc(Nc3ccc(F)cc3)c2)NC(=O)C1Sc1ccccc1Cl] | 3-(2-chloro- phenyl) sulfanyl-6- [3-(4-fluoro anilino) phenyl]-6- (3-thienyl) piperidine- 2,4-dione was prepared according to example 417 | M + H = 523 |
| 435 | MD | 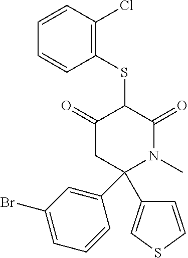 [SMILES: CN1C(=O)C(Sc2ccccc2Cl)C(=O)CC1(c1ccsc1)c1cccc(Br)c1] | 6-(3-bromo- phenyl)-3- (2-chloro- phenyl) sulfanyl-1- methyl-6- (3-thienyl) piperidine- 2,4-dione was prepared as in example 11. | H NMR (400 MHz, DMSO- d6) δ 11.40 (s, 1H), 7.75-7.62 (m, 2H), 7.49-7.29 (m, 4H), 7.18 (d, J = 4.0 Hz, 2H), 7.01 (td, J = 7.6, 1.6 Hz, 1H), 6.94- 6.87 (m, 1H), 6.12 (dd, J = 8.0, 1.5 Hz, 1H), 3.78-3.50 (m, 2H), 2.67 (s, 3H) |
| 436 | MD | 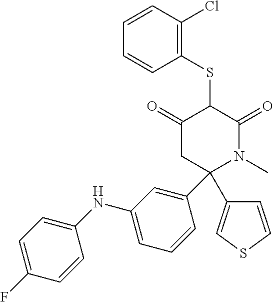 [SMILES: CN1C(=O)C(Sc2ccccc2Cl)C(=O)CC1(c1ccsc1)c1cccc(Nc2ccc(F)cc2)c1] | 3-(2-chloro- phenyl) sulfanyl-6- [3-(4-fluoro anilino) phenyl]-1- methyl-6- (3-thienyl) piperidine- 2,4-dione was prepared according to example 11 | 1H NMR (400 MHz, DMSO- d6) δ 11.33 (s, 1H), 8.24 (s, 1H), 7.76-7.61 (m, 1H), 7.36-7.13 (m, 5H), 7.11-7.00 (m, 5H), 6.91-6.81 (m, 2H), 6.80-6.71 (m, 1H), 6.18 (dd, J = 8.0, 1.6 Hz, 1H), 3.69 (d, J = 16.6 Hz, 1H), 3.48 (s, 1H), 2.69 (s, 3H) |
| 437 | MD | 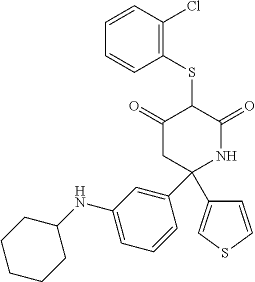 [SMILES: O=C1CC(c2ccsc2)(c2cccc(NC3CCCCC3)c2)NC(=O)C1Sc1ccccc1Cl] | 3-(2-chloro- phenyl) sulfanyl-6- [3-(cyclo- hexylamino) phenyl]-6- (3-thienyl) piperidine- 2,4-dione was prepared according to example 417 | 1H NMR (400 MHz, DMSO- d6) δ 11.39 (s, 1H), 8.33 (s, 1H), 7.57 (dd, J = 5.1, 2.9 Hz, 1H), 7.41-7.34 (m, 1H), 7.29 (dd, J = 8.0, 1.3 Hz, 1H), 7.18 (dd, J = 5.1, 1.4 Hz, 1H), 7.07- 6.94 (m, 2H), 6.78 (td, J = 7.7, 1.4 Hz, 1H), 6.59 (s, 1H), 6.51 (d, J = 8.0 Hz, 2H), 5.95 (dd, J = 8.0, 1.5 Hz, 1H), 5.43 (s, 1H), 3.50-3.35 (m, 2H), 1.86 (t, J = 14.3 Hz, 2H), 1.74-1.64 (m, 2H), 1.64-1.51 (m, 1H), 1.34-1.24 (m, 2H), 1.21-1.02 (m, H) |
| 438 | MD | 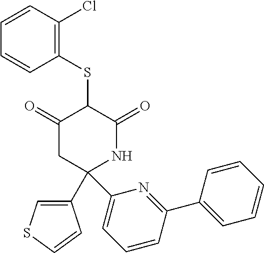 [SMILES: O=C1CC(c2ccsc2)(c2cccc(-c3ccccc3)n2)NC(=O)C1Sc1ccccc1Cl] | 3-(2-chloro- phenyl) sulfanyl-6- (6-phenyl- 2-pyridyl)- 6-(3-thienyl) piperidine- 2,4-dione was prepared according to example 418 with using phenyl- boronic acid | 1H NMR (400 MHz, DMSO- d6) δ 11.68 (s, 1H), 8.62 (s, 1H), 8.24-8.13 (m, 2H), 8.03- 7.93 (m, 2H), 7.71-7.61 (m, 1H), 7.56-7.43 (m, 4H), 7.37 (dd, J = 3.0, 1.4 Hz, 1H), 7.25 (dd, J = 8.0, 1.3 Hz, 1H), 7.18 (dd, J = 5.1, 1.4 Hz, 1H), 7.01- 6.77 (m, 1H), 6.66-6.50 (m, 1H), 5.81 (dd, J = 8.0, 1.5 Hz, 1H), 4.15 (d, J = 16.2 Hz, 1H), 3.41 (d, J = 16.2 Hz, 1H) |
| 439 | MD | 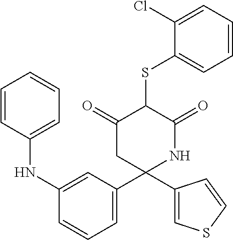 [SMILES: O=C1CC(c2ccsc2)(c2cccc(Nc3ccccc3)c2)NC(=O)C1Sc1ccccc1Cl] | 6-(3-anilino- phenyl)-3- (2-chloro- phenyl) sulfanyl-6- (3-thienyl) piperidine- 2,4-dione was prepared according to example 417 | 1H NMR (400 MHz, DMSO- d6) δ 8.19 (s, 1H), 8.13 (s, 1H), 7.57 (dd, J = 5.1, 2.9 Hz, 1H), 7.38 (dd, J = 3.0, 1.4 Hz, 1H), 7.28-7.14 (m, 6H), 7.03-6.85 (m, 5H), 6.82-6.74 (m, 2H), 6.00 (dd, J = 8.0, 1.5 Hz, 1H). |
| 440 | MD | 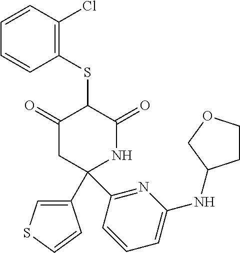 [SMILES: O=C1CC(c2ccsc2)(c2cccc(NC3CCOC3)n2)NC(=O)C1Sc1ccccc1Cl] | 3-(2-chloro- phenyl) sulfanyl-6- [6-(tetrahydro- furan-3- ylamino)-2- pyridyl]-6- (3-thienyl) piperidine- 2,4-dione was prepared according to example 417 | 1H NMR (400 MHz, DMSO- d6) δ 11.46 (s, 1H), 8.14 (s, 1H), 7.48 (ddd, J = 5.0, 2.9, 1.9 Hz, 1H), 7.45-7.38 (m, 1H), 7.37-7.32 (m, 1H), 7.29- 7.25 (m, 1H), 7.23-7.12 (m, 1H), 6.98-6.92 (m, 1H), 6.88- 6.83 (m, 1H), 6.79-6.73 (m, 2H), 6.43 (d, J = 8.2 Hz, 1H), 6.03-5.95 (m, 1H), 4.46-4.34 (m, 1H), 3.95-3.67 (m, 4H), 3.49-3.41 (m, 1H), 2.24-2.11 (m, 1H), 1.82-1.70 (m, 1H) |
| 441 | MD | 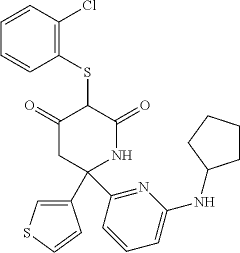 [SMILES: O=C1CC(c2ccsc2)(c2cccc(NC3CCCC3)n2)NC(=O)C1Sc1ccccc1Cl] | 3-(2-chloro- phenyl) sulfanyl-6- [6-(cyclo- pentylamino)- 2-pyridyl]-6- (3-thienyl) piperidine- 2,4-dione was prepared according to example 417 | 1H NMR (400 MHz, DMSO- d6) δ 11.41 (s, 1H), 8.23 (s, 1H), 7.56-7.47 (m, 1H), 7.40- 7.33 (m, 2H), 7.33-7.26 (m, 1H), 7.22-7.13 (m, 1H), 7.04- 6.90 (m, 1H), 6.85-6.76 (m, 1H), 6.69 (d, J = 7.4 Hz, 1H), 6.56 (d, J = 6.5 Hz, 1H), 6.38 (d, J = 8.3 Hz, 1H), 6.02 (dd, J = 8.0, 1.5 Hz, 1H), 4.15 (q, J = 6.6 Hz, 1H), 3.96-3.77 (m, 2H), 1.97-1.88 (m, 2H), 1.69- 1.34 (m, 6H). |
| 442 | MD | 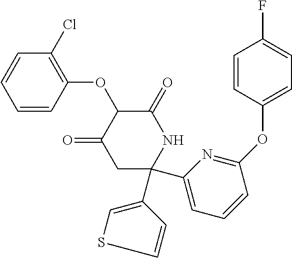 [SMILES: O=C1CC(c2ccsc2)(c2cccc(Oc3ccc(F)cc3)n2)NC(=O)C1Oc1ccccc1Cl] | 3-(2-chloro- phenoxy)-6- [6-(4-fluoro- phenoxy)-2- pyridyl]-6- (3-thienyl) piperidine- 2,4-dione was prepared according to example 419 | 1H NMR (400 MHz, DMSO- d6) δ 7.96-7.86 (m, 1H), 7.49- 7.33 (m, 3H), 7.30-7.14 (m, 5H), 7.01 (dd, J = 5.1, 1.4 Hz, 1H), 6.97-6.82 (m, 3H), 6.10 (dd, J = 8.1, 1.6 Hz, 1H), 3.42 (d, J = 16.0 Hz, 1H), 3.15 (d, J = 16.3 Hz, 1H) |
| 443 | MD | 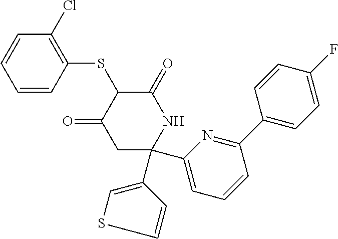 [SMILES: O=C1CC(c2ccsc2)(c2cccc(-c3ccc(F)cc3)n2)NC(=O)C1Sc1ccccc1Cl] | 3-(2-chloro- phenyl) sulfanyl-6- [6-(4-fluoro- phenyl)-2- pyridyl]-6- (3-thienyl) piperidine- 2,4-dione was prepared according to example 418 with using 4-F-phenyl- boronic acid) | 1H NMR (400 MHz, DMSO- d6) δ 11.68 (s, 1H), 8.59 (s, 1H), 8.31-8.17 (m, 2H), 8.03- 7.92 (m, 2H), 7.64 (dd, J = 5.8, 2.9 Hz, 1H), 7.52 (dd, J = 5.1, 2.9 Hz, 1H), 7.40- 7.25 (m, 4H), 7.17 (dd, J = 5.1, 1.4 Hz, 1H), 6.89 (td, J = 7.6, 1.5 Hz, 1H), 6.62- 6.55 (m, 1H), 5.81 (dd, J = 8.0, 1.5 Hz, 1H), 4.12 (d, J = 16.2 Hz, 1H), 3.41 (d, J = 16.2 Hz, 1H). |
| 444 | MD | 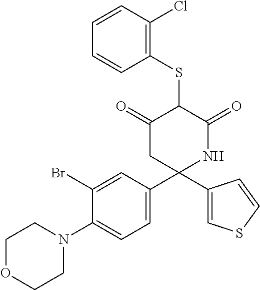 [SMILES: O=C1CC(c2ccsc2)(c2ccc(N3CCOCC3)c(Br)c2)NC(=O)C1Sc1ccccc1Cl] | 6-(3-bromo- 4-morpholino- phenyl)-3- (2-chloro- phenyl) sulfanyl-6- (3-thienyl) piperidine- 2,4-dione (see example. 8) | H NMR (400 MHz, DMSO- d6) δ 8.47 (s, 1H), 7.66 (d, J = 2.2 Hz, 1H), 7.59 (dd, J = 5.0, 2.9 Hz, 1H), 7.39- 7.35 (m, 2H), 7.30 (dd, J = 7.9, 1.3 Hz, 1H), 7.20-7.14 (m, 2H), 7.02-6.94 (m, 1H), 6.81-6.74 (m, 1H), 5.94 (dd, J = 8.0, 1.5 Hz, 1H), 3.78- 3.70 (m, 4H), 3.49-3.39 (m, 2H), 3.01-2.93 (m, 4H) |
| 445 | MD | 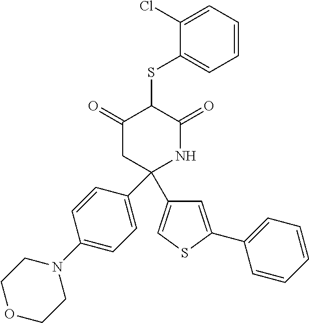 [SMILES: O=C1CC(c2ccc(N3CCOCC3)cc2)(c2csc(-c3ccccc3)c2)NC(=O)C1Sc1ccccc1Cl] | 3-(2-chloro- phenyl) sulfanyl-6- (4-morpholino- phenyl)-6- (5-phenyl- 3-thienyl) piperidine- 2,4-dione (see example 9) | 1H NMR (400 MHz, DMSO- d6) δ 8.37 (s, 1H), 7.61 (dd, J = 7.7, 1.5 Hz, 3H), 7.45- 7.39 (m, 2H), 7.34-7.25 (m, 5H), 7.02-6.89 (m, 3H), 6.76- 6.67 (m, 1H), 6.02 (dd, J = 8.0, 1.5 Hz, 1H), 3.78-3.71 (m, 4H), 3.48 (d, J = 16.7 Hz, 2H), 3.16-3.09 (m, 4H) |
| 446 | MD | 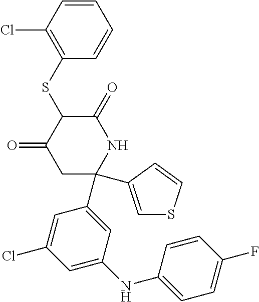 [SMILES: O=C1CC(c2ccsc2)(c2cc(Cl)cc(Nc3ccc(F)cc3)c2)NC(=O)C1Sc1ccccc1Cl] | 6-[3-chloro- 5-(4-fluoro- anilino) phenyl]-3- (2-chloro- phenyl) sulfanyl-6- (3-thienyl) piperidine- 2,4-dione (MD) was prepared according to example 417 | 1H NMR (400 MHz, DMSO- d6) δ 11.51 (s, 1H), 8.44 (s, 1H), 8.41 (s, 1H), 7.61 (dd, J = 5.1, 2.9 Hz, 1H), 7.40 (dd, J = 2.9, 1.4 Hz, 1H), 7.30 (dd, J = 7.9, 1.3 Hz, 1H), 7.19 (dd, J = 5.1, 1.4 Hz, 1H), 7.12-6.96 (m, 6H), 6.91- 6.79 (m, 3H), 6.01 (dd, J = 7.9, 1.5 Hz, 1H), 3.47-3.35 (m, 2H) |
| 447 | SS | 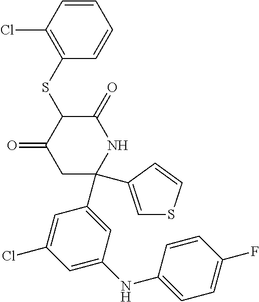 [SMILES: O=C1CC(c2ccsc2)(c2cc(Cl)cc(Nc3ccc(F)cc3)c2)NC(=O)C1Sc1ccccc1Cl] | 6-[3-chloro- 5-(4-fluoro- anilino) phenyl]-3- (2-chloro- phenyl) sulfanyl-6- (3-thienyl) piperidine- 2,4-dione (SS) | M + H = 557 |
| 448 | MD | 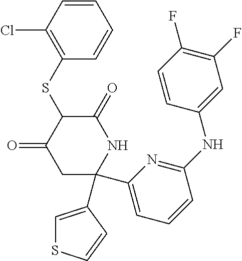 [SMILES: O=C1CC(c2ccsc2)(c2cccc(Nc3ccc(F)c(F)c3)n2)NC(=O)C1Sc1ccccc1Cl] | 3-(2-chloro- phenyl) sulfanyl-6- [6-(3,4- difluoro- anilino)-2- pyridyl]-6- (3-thienyl) piperidine- 2,4-dione was prepared according to example 417 | 1H NMR (400 MHz, DMSO- d6) δ 11.48 (s, 1H), 9.33 (s, 1H), 8.40 (s, 1H), 7.83-7.73 (m, 1H), 7.66 (d, J = 8.1 Hz, 1H), 7.54 (dd, J = 5.1, 2.9 Hz, 1H), 7.41 (dd, J = 3.0, 1.4 Hz, 1H), 7.34-7.24 (m, 3H), 7.20 (dd, J = 5.1, 1.4 Hz, 1H), 7.08 (d, J = 7.5 Hz, 1H), 6.98 (td, J = 7.6, 1.5 Hz, 1H), 6.85- 6.80 (m, 1H), 6.75 (d, J = 8.2 Hz, 1H), 6.06 (dd, J = 8.0, 1.5 Hz, 1H), 3.74 (d, J = 16.5 Hz, 1H), 3.49 (d, J = 16.5 Hz, 1H). |
| 449 | MD | 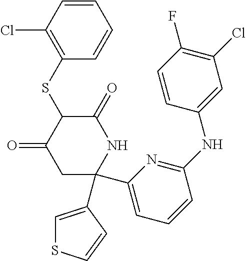 [SMILES: O=C1CC(c2ccsc2)(c2cccc(Nc3ccc(F)c(Cl)c3)n2)NC(=O)C1Sc1ccccc1Cl] | 6-[6-(3- chloro-4- fluoro- anilino)-2- pyridyl]-3- (2-chloro- phenyl) sulfanyl-6- (3-thienyl) piperidine- 2,4-dione was prepared according to example 417 | M + H = 558 |
| 450 | SS | 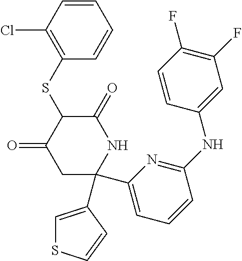 [SMILES: O=C1CC(c2ccsc2)(c2cccc(Nc3ccc(F)c(F)c3)n2)NC(=O)C1Sc1ccccc1Cl] | 3-(2-chloro- phenyl) sulfanyl-6- [6-(3,4- difluoro- anilino)-2- pyridyl]-6- (3-thienyl) piperidine- 2,4-dione (SS) was prepared according to example 417 | M + H = 542 |
| 451 | SS | 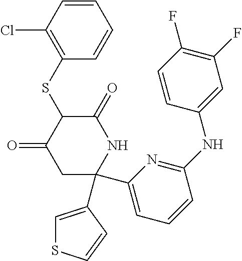 [SMILES: O=C1CC(c2ccsc2)(c2cccc(Nc3ccc(F)c(F)c3)n2)NC(=O)C1Sc1ccccc1Cl] | 3-(2-chloro- phenyl) sulfanyl-6- [6-(3,4- difluoro- anilino)-2- pyridyl]-6- (3-thienyl) piperidine- 2,4-dione (SS) was prepared according to example 417 | M + H = 542 |
| 452 | SS | 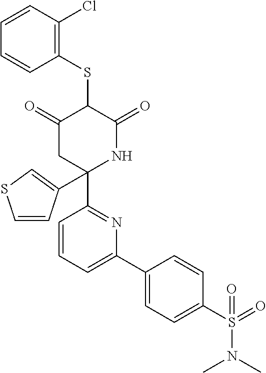 [SMILES: CN(C)S(=O)(=O)c1ccc(-c2cccc(C3(c4ccsc4)CC(=O)C(Sc4ccccc4Cl)C(=O)N3)n2)cc1] | 4-[6-[5-(2- chlorophenyl) sulfanyl-4,6- dioxo-2-(3- thienyl)-2- piperidyl]-2- pyridyl]-N,N- dimethyl- benzene- sulfonamide (SS) was prepared according to example 418 | M + H = 598 |
| 453 | SS | 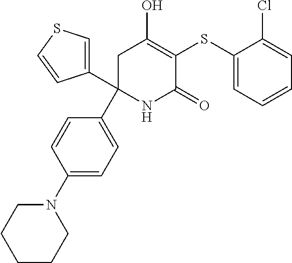 [SMILES: O=C1NC(c2ccc(N3CCCCC3)cc2)(c2ccsc2)CC(O)=C1Sc1ccccc1Cl] | 5-(2-chloro- phenyl) sulfanyl-4- hydroxy-2- [4-(1- piperidyl) phenyl]-2- (3-thienyl)- 1,3-dihydro- pyridin-6- one was prepared as described in example 56. | |
| 454 | SS | 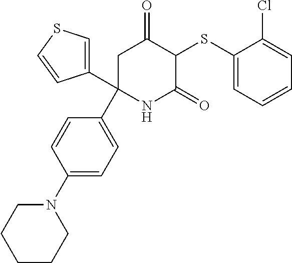 [SMILES: O=C1CC(c2ccc(N3CCCCC3)cc2)(c2ccsc2)NC(=O)C1Sc1ccccc1Cl] | 3-(2-chloro- phenyl) sulfanyl-6- [4-(1- piperidyl) phenyl]-6- (3-thienyl) piperidine- 2,4-dione was prepared as described in example 56. | |

*ST: Stereochemistry: SS = Single Stereoisomer; MD = Mixture of Diastereomers

### LDHA Enzyme Inhibition Assay Protocol

Human recombinant carboxy-terminal his-tagged LDHA (amino acids 2-332) was expressed and purified from E. coli. The enzyme assay was performed in uClear low volume 384-well plates (Greiner #788092), 10 μL volume with the following final enzyme and buffer conditions: 50 mM Hepes (pH 7.2), 0.01% (v/v) TritonX-100, 0.01% (0.1 mg/mL) Bovine Gamma Globulin, 2 mM DTT, 1 nM LDHA, 50 M NADH, and 50 M pyruvate. Test compounds were diluted in 100% DMSO with 1:3 serial dilutions. Oxamate (Sigma #02751) was used as a positive control and was diluted in H₂O (10-point 1:3 serial dilutions, final DMSO 1%). For the enzyme reaction, serially diluted compounds were added to a mixture of enzyme and NADH. The assay plates were then incubated at room temperature for 10 minutes and a baseline read was conducted on the FDSS700 (Hamamatsu) with excitation at 340 nm and emission at 480 nm for 12.5 seconds to identify any compounds which interfere with NADH fluorescence. Following the baseline read, pyruvate was added to the assay plates and the plates were read with excitation 340 nm and emission 480 nm for 10 minutes every 2.5 seconds. A suitable linear timeframe was selected (150-400 s) to calculate the slope of each concentration tested. The raw data were fitted to 4-parameter dose-response curves using the following equation:

fit=(A+((B−A)/(1+((C/x)̂D))))

inv=(C/((((B−A)/(y−A))−)̂(1/D)))

res=(y−fit)
where A=minimum y, B=maximum y, C=50% y max, and D=slope factor.

The curve bottom was set to the background rate (initial 5 second recording prior to addition of pyruvate) and curve top was set to no inhibitor (DMSO only) control wells rate. Oxamate was used as a positive control and exhibited a mean IC₅₀ value of 57.2 μM±13.1 μM (n=202). For previous descriptions of LDH enzyme assays, see: Rossmann, M. G. et al. Evolutionary and structural relationships among dehydrogenases. In: Boyer, P. D. Ed., The Enzymes, vol. XI. New York: Academic Press, 1975; pp 61-102. See also the Supplementary Material section of: Moorhouse, A. D. et a. Chem. Commun. 2011, 47, 230.

The compounds of the present invention were tested for their capacity to inhibit LDHA activity and activation as described in the enzyme inhibition assay described above. The following table summarizes the results of this assay by reference to the exemplified compounds of the invention:

| Ex No. | Stereochemistry | LDHA IC₅₀ (μM) |
|---|---|---|
| 1 | Mixture of Diastereomers | 0.006 |
| 2 | Mixture of Diastereomers | 0.082 |
| 3 | Mixture of Diastereomers | 0.003 |
| 3 | Mixture of Diastereomers | 0.123 |
| 3 | Mixture of Diastereomers | 0.569 |
| 4 | Mixture of Diastereomers | 0.003 |
| 4 | Mixture of Diastereomers | 0.003 |
| 4 | Mixture of Diastereomers | 0.211 |
| 5 | Mixture of Diastereomers | 0.028 |
| 5 | Mixture of Diastereomers | 0.009 |
| 5 | Mixture of Diastereomers | 0.066 |
| 6 | Single Stereoisomer | 0.008 |
| 6 | Single Stereoisomer | 0.002 |
| 6 | Mixture of Diastereomers | 0.165 |
| 7 | Mixture of Diastereomers | 0.009 |
| 8 | Mixture of Diastereomers | 0.004 |
| 9 | Single Stereoisomer | 0.026 |
| 10 | Mixture of Diastereomers | 0.013 |
| 10 | Mixture of Diastereomers | 0.042 |
| 10 | Single Stereoisomer | 0.004 |
| 11 | Single Stereoisomer | 0.002 |
| 12 | Mixture of Diastereomers | 0.027 |
| 13 | Mixture of Diastereomers | 0.058 |
| 14 | Mixture of Diastereomers | 0.098 |
| 14 | Mixture of Diastereomers | 0.004 |
| 14 | Mixture of Diastereomers | 0.063 |
| 15 | Mixture of Diastereomers | 0.016 |
| 15 | Mixture of Diastereomers | 0.092 |
| 15 | Mixture of Diastereomers | 0.022 |
| 15 | Mixture of Diastereomers | 0.043 |
| 15 | Mixture of Diastereomers | 0.008 |
| 16 | Mixture of Diastereomers | 0.105 |
| 16 | Mixture of Diastereomers | 0.076 |
| 17 | Mixture of Diastereomers | 0.114 |
| 17 | Mixture of Diastereomers | 0.023 |
| 17 | Mixture of Diastereomers | 1.270 |
| 18 | Mixture of Diastereomers | 0.012 |
| 19 | Single Stereoisomer | 0.006 |
| 19 | Single Stereoisomer | 0.203 |
| 19 | Mixture of Diastereomers | 0.005 |
| 20 | Mixture of Diastereomers | 0.039 |
| 21 | Mixture of Diastereomers | 0.119 |
| 22 | Mixture of Diastereomers | 0.086 |
| 23 | Mixture of Diastereomers | 0.026 |
| 24 | Mixture of Diastereomers | 0.018 |
| 25 | Mixture of Diastereomers | 0.023 |
| 26 | Mixture of Diastereomers | 0.038 |
| 27 | Mixture of Diastereomers | 0.050 |
| 28 | Mixture of Diastereomers | 0.031 |
| 29 | Mixture of Diastereomers | 0.009 |
| 30 | Mixture of Diastereomers | 0.053 |
| 31 | Mixture of Diastereomers | 0.008 |
| 32 | Mixture of Diastereomers | 0.010 |
| 33 | Mixture of Diastereomers | 0.007 |
| 34 | Mixture of Diastereomers | 0.033 |
| 35 | Mixture of Diastereomers | 0.007 |
| 36 | Mixture of Diastereomers | 0.021 |
| 36 | Mixture of Diastereomers | 0.008 |
| 36 | Mixture of Diastereomers | 0.025 |
| 37 | Mixture of Diastereomers | 0.034 |
| 37 | Mixture of Diastereomers | 0.029 |
| 37 | Mixture of Diastereomers | 0.040 |
| 38 | Mixture of Diastereomers | 0.004 |
| 39 | Mixture of Diastereomers | 0.003 |
| 39 | Mixture of Diastereomers | 0.012 |
| 40 | Mixture of Diastereomers | 0.036 |
| 41 | Mixture of Diastereomers | 0.016 |
| 42 | Mixture of Diastereomers | 0.005 |
| 42 | Mixture of Diastereomers | 0.010 |
| 42 | Mixture of Diastereomers | 0.017 |
| 43 | Mixture of Diastereomers | 0.017 |
| 44 | Mixture of Diastereomers | 0.022 |
| 45 | Mixture of Diastereomers | 0.069 |
| 46 | Mixture of Diastereomers | 0.005 |
| 47 | Mixture of Diastereomers | 0.120 |
| 48 | Mixture of Diastereomers | 0.052 |
| 49 | Mixture of Diastereomers | 0.025 |
| 50 | Mixture of Diastereomers | 0.022 |
| 51 | Mixture of Diastereomers | 0.149 |
| 52 | Mixture of Diastereomers | 0.045 |
| 53 | Mixture of Diastereomers | 0.011 |
| 54 | Mixture of Diastereomers | 0.201 |
| 55 | Mixture of Diastereomers | 0.029 |
| 56 | Mixture of Diastereomers | 0.007 |
| 57 | Mixture of Diastereomers | 0.086 |
| 58 | Mixture of Diastereomers | 0.010 |
| 59 | Mixture of Diastereomers | 0.056 |
| 60 | Mixture of Diastereomers | 0.009 |
| 61 | Mixture of Diastereomers | 0.020 |
| 62 | Mixture of Diastereomers | 0.036 |
| 63 | Mixture of Diastereomers | 0.037 |
| 64 | Mixture of Diastereomers | 0.126 |
| 65 | Mixture of Diastereomers | 0.056 |
| 66 | Mixture of Diastereomers | 0.089 |
| 67 | Mixture of Diastereomers | 0.224 |
| 68 | Mixture of Diastereomers | 0.008 |
| 69 | Single Stereoisomer | 0.004 |
| 70 | Single Stereoisomer | 0.104 |
| 71 | Mixture of Diastereomers | 0.056 |
| 72 | Mixture of Diastereomers | 0.025 |
| 73 | Single Stereoisomer | 0.111 |
| 74 | Single Stereoisomer | 0.014 |
| 75 | Mixture of Diastereomers | 0.070 |
| 76 | Mixture of Diastereomers | 0.015 |
| 77 | Mixture of Diastereomers | 0.031 |
| 78 | Single Stereoisomer | 0.023 |
| 79 | Single Stereoisomer | 0.199 |
| 80 | Mixture of Diastereomers | 0.056 |
| 81 | Mixture of Diastereomers | 0.229 |
| 82 | Mixture of Diastereomers | 0.049 |
| 83 | Mixture of Diastereomers | 0.013 |
| 84 | Mixture of Diastereomers | 0.008 |
| 85 | Mixture of Diastereomers | 0.018 |
| 86 | Mixture of Diastereomers | 0.022 |
| 87 | Mixture of Diastereomers | 0.010 |
| 88 | Mixture of Diastereomers | 0.017 |
| 89 | Mixture of Diastereomers | 0.018 |
| 90 | Mixture of Diastereomers | 0.013 |
| 91 | Mixture of Diastereomers | 0.011 |
| 92 | Single Stereoisomer | 0.007 |
| 93 | Single Stereoisomer | 0.319 |
| 94 | Single Stereoisomer | 0.023 |
| 95 | Single Stereoisomer | 0.290 |
| 96 | Mixture of Diastereomers | 0.019 |
| 97 | Mixture of Diastereomers | 0.030 |
| 98 | Mixture of Diastereomers | 0.167 |
| 99 | Mixture of Diastereomers | 0.006 |
| 100 | Single Stereoisomer | 0.046 |
| 101 | Mixture of Diastereomers | 0.018 |
| 102 | Mixture of Diastereomers | 0.018 |
| 103 | Mixture of Diastereomers | 0.141 |
| 104 | Mixture of Diastereomers | 0.118 |
| 105 | Single Stereoisomer | 0.125 |
| 106 | Single Stereoisomer | 0.732 |
| 107 | Mixture of Diastereomers | 0.131 |
| 108 | Mixture of Diastereomers | 0.108 |
| 109 | Mixture of Diastereomers | 0.010 |
| 110 | Mixture of Diastereomers | 0.018 |
| 111 | Mixture of Diastereomers | 0.012 |
| 112 | Mixture of Diastereomers | 0.010 |
| 113 | Single Stereoisomer | 0.014 |
| 114 | Single Stereoisomer | 0.053 |
| 115 | Single Stereoisomer | 0.031 |
| 116 | Single Stereoisomer | 0.376 |
| 117 | Mixture of Diastereomers | 0.076 |
| 118 | Single Stereoisomer | 0.622 |
| 119 | Single Stereoisomer | 0.293 |
| 120 | Mixture of Diastereomers | 0.016 |
| 121 | Mixture of Diastereomers | 0.008 |
| 122 | Single Stereoisomer | 0.022 |
| 123 | Single Stereoisomer | 0.006 |
| 124 | Mixture of Diastereomers | 0.003 |
| 125 | Mixture of Diastereomers | 0.003 |
| 126 | Mixture of Diastereomers | 0.238 |
| 127 | Mixture of Diastereomers | 0.008 |
| 128 | Mixture of Diastereomers | 0.024 |
| 129 | Mixture of Diastereomers | 0.020 |
| 130 | Mixture of Diastereomers | 0.005 |
| 131 | Mixture of Diastereomers | 0.046 |
| 132 | Mixture of Diastereomers | 0.009 |
| 133 | Mixture of Diastereomers | 0.089 |
| 134 | Single Stereoisomer | 0.008 |
| 135 | Mixture of Diastereomers | 0.040 |
| 136 | Single Stereoisomer | 0.222 |
| 137 | Mixture of Diastereomers | 0.073 |
| 138 | Mixture of Diastereomers | 0.025 |
| 139 | Mixture of Diastereomers | 0.009 |
| 140 | Mixture of Diastereomers | 0.012 |
| 141 | Single Stereoisomer | 0.003 |
| 142 | Single Stereoisomer | 0.031 |
| 143 | Mixture of Diastereomers | 0.033 |
| 144 | Single Stereoisomer | 0.143 |
| 145 | Single Stereoisomer | 0.088 |
| 146 | Single Stereoisomer | 0.189 |
| 147 | Single Stereoisomer | 0.008 |
| 148 | Mixture of Diastereomers | 0.014 |
| 149 | Mixture of Diastereomers | 0.069 |
| 150 | Mixture of Diastereomers | 0.019 |
| 151 | Mixture of Diastereomers | 0.015 |
| 152 | Mixture of Diastereomers | 0.006 |
| 153 | Single Stereoisomer | 0.012 |
| 154 | Mixture of Diastereomers | 0.027 |
| 155 | Mixture of Diastereomers | 0.025 |
| 156 | Single Stereoisomer | 0.072 |
| 157 | Mixture of Diastereomers | 0.030 |
| 158 | Mixture of Diastereomers | 0.018 |
| 159 | Mixture of Diastereomers | 0.004 |
| 160 | Mixture of Diastereomers | 0.008 |
| 161 | Single Stereoisomer | 0.019 |
| 162 | Single Stereoisomer | 0.004 |
| 163 | Single Stereoisomer | 0.194 |
| 164 | Single Stereoisomer | 0.009 |
| 165 | Single Stereoisomer | 0.004 |
| 166 | Mixture of Diastereomers | 0.004 |
| 167 | Mixture of Diastereomers | 0.006 |
| 168 | Single Stereoisomer | 0.190 |
| 169 | Mixture of Diastereomers | 0.010 |
| 170 | Single Stereoisomer | 0.145 |
| 171 | Single Stereoisomer | 0.101 |
| 172 | Single Stereoisomer | 0.056 |
| 173 | Single Stereoisomer | 0.183 |
| 174 | Single Stereoisomer | 0.068 |
| 175 | Single Stereoisomer | 0.013 |
| 176 | Mixture of Diastereomers | 0.010 |
| 177 | Mixture of Diastereomers | 0.016 |
| 178 | Single Stereoisomer | 0.155 |
| 179 | Single Stereoisomer | 0.014 |
| 180 | Single Stereoisomer | 0.002 |
| 181 | Single Stereoisomer | 0.006 |
| 182 | Mixture of Diastereomers | 0.056 |
| 183 | Single Stereoisomer | 0.005 |
| 184 | Single Stereoisomer | 0.002 |
| 185 | Mixture of Diastereomers | 0.055 |
| 186 | Mixture of Diastereomers | 0.021 |
| 187 | Mixture of Diastereomers | 0.025 |
| 188 | Mixture of Diastereomers | 0.015 |
| 189 | Single Stereoisomer | 0.150 |
| 190 | Single Stereoisomer | 0.092 |
| 191 | Single Stereoisomer | 0.158 |
| 192 | Single Stereoisomer | 0.232 |
| 193 | Single Stereoisomer | 0.059 |
| 194 | Single Stereoisomer | 0.005 |
| 195 | Single Stereoisomer | 0.143 |
| 196 | Single Stereoisomer | 0.036 |
| 197 | Single Stereoisomer | 0.010 |
| 198 | Single Stereoisomer | 0.177 |
| 199 | Single Stereoisomer | 0.012 |
| 200 | Mixture of Diastereomers | 0.229 |
| 201 | Single Stereoisomer | 0.037 |
| 202 | Single Stereoisomer | 0.018 |
| 203 | Single Stereoisomer | 0.044 |
| 204 | Mixture of Diastereomers | 0.007 |
| 205 | Mixture of Diastereomers | 0.058 |
| 206 | Mixture of Diastereomers | 0.027 |
| 207 | Mixture of Diastereomers | 0.020 |
| 208 | Mixture of Diastereomers | 0.149 |
| 209 | Mixture of Diastereomers | 0.006 |
| 210 | Mixture of Diastereomers | 0.008 |
| 211 | Mixture of Diastereomers | 0.183 |
| 212 | Mixture of Diastereomers | 0.030 |
| 213 | Mixture of Diastereomers | 0.034 |
| 214 | Mixture of Diastereomers | 0.017 |
| 215 | Mixture of Diastereomers | 0.014 |
| 216 | Single Stereoisomer | 0.002 |
| 217 | Single Stereoisomer | 0.010 |
| 218 | Single Stereoisomer | 0.117 |
| 219 | Single Stereoisomer | 0.240 |
| 220 | Single Stereoisomer | 0.162 |
| 221 | Single Stereoisomer | 0.320 |
| 222 | Single Stereoisomer | 0.322 |
| 223 | Single Stereoisomer | 0.057 |
| 224 | Mixture of Diastereomers | 0.020 |
| 225 | Mixture of Diastereomers | 0.103 |
| 226 | Single Stereoisomer | 0.020 |
| 227 | Single Stereoisomer | 0.015 |
| 228 | Single Stereoisomer | 0.419 |
| 229 | Mixture of Diastereomers | 0.011 |
| 230 | Mixture of Diastereomers | 0.022 |
| 231 | Mixture of Diastereomers | 0.009 |
| 232 | Mixture of Diastereomers | 0.009 |
| 233 | Mixture of Diastereomers | 0.017 |
| 234 | Mixture of Diastereomers | 0.004 |
| 235 | Mixture of Diastereomers | 0.021 |
| 236 | Mixture of Diastereomers | 0.025 |
| 237 | Single Stereoisomer | 0.094 |
| 238 | Mixture of Diastereomers | 0.018 |
| 239 | Single Stereoisomer | 0.015 |
| 240 | Single Stereoisomer | 0.003 |
| 241 | Single Stereoisomer | 0.003 |
| 242 | Mixture of Diastereomers | 0.004 |
| 243 | Single Stereoisomer | 0.001 |
| 244 | Single Stereoisomer | 0.023 |
| 245 | Mixture of Diastereomers | 0.018 |
| 246 | Single Stereoisomer | 0.010 |
| 247 | Single Stereoisomer | 0.010 |
| 248 | Single Stereoisomer | 0.005 |
| 249 | Mixture of Diastereomers | 0.099 |
| 250 | Single Stereoisomer | 0.036 |
| 251 | Single Stereoisomer | 0.019 |
| 252 | Mixture of Diastereomers | 0.079 |
| 253 | Mixture of Diastereomers | 0.081 |
| 254 | Single Stereoisomer | 0.047 |
| 255 | Mixture of Diastereomers | 0.102 |
| 256 | Single Stereoisomer | 0.062 |
| 257 | Mixture of Diastereomers | 0.016 |
| 258 | Mixture of Diastereomers | 0.087 |
| 259 | Single Stereoisomer | 0.011 |
| 260 | Single Stereoisomer | 0.006 |
| 261 | Single Stereoisomer | 0.039 |
| 262 | Mixture of Diastereomers | 0.104 |
| 263 | Mixture of Diastereomers | 0.086 |
| 264 | Mixture of Diastereomers | 0.077 |
| 265 | Mixture of Diastereomers | 0.022 |
| 266 | Single Stereoisomer | 0.099 |
| 267 | Single Stereoisomer | 1.640 |
| 268 | Single Stereoisomer | 0.402 |
| 269 | Single Stereoisomer | 0.073 |
| 270 | Single Stereoisomer | 0.099 |
| 271 | Single Stereoisomer | 0.355 |
| 272 | Single Stereoisomer | 0.459 |
| 273 | Single Stereoisomer | 0.072 |
| 274 | Single Stereoisomer | 0.157 |
| 275 | Single Stereoisomer | 0.233 |
| 276 | Single Stereoisomer | 0.175 |
| 277 | Single Stereoisomer | 0.337 |
| 278 | Single Stereoisomer | 0.339 |
| 279 | Single Stereoisomer | 0.272 |
| 280 | Single Stereoisomer | 0.311 |
| 281 | Single Stereoisomer | 0.189 |
| 282 | Mixture of Diastereomers | 0.012 |
| 283 | Mixture of Diastereomers | 0.037 |
| 284 | Mixture of Diastereomers | 0.041 |
| 285 | Single Stereoisomer | 0.010 |
| 286 | Mixture of Diastereomers | 0.057 |
| 287 | Mixture of Diastereomers | 0.010 |
| 288 | Mixture of Diastereomers | 0.141 |
| 289 | Mixture of Diastereomers | 0.022 |
| 290 | Mixture of Diastereomers | 0.017 |
| 291 | Single Stereoisomer | 0.093 |
| 292 | Mixture of Diastereomers | 0.037 |
| 293 | Mixture of Diastereomers | 0.091 |
| 294 | Mixture of Diastereomers | 0.036 |
| 295 | Single Stereoisomer | 0.081 |
| 296 | Mixture of Diastereomers | 0.039 |
| 297 | Single Stereoisomer | 0.149 |
| 298 | Single Stereoisomer | 0.063 |
| 300 | Mixture of Diastereomers | 0.144 |
| 301 | Mixture of Diastereomers | 0.092 |
| 302 | Mixture of Diastereomers | 0.030 |
| 303 | Single Stereoisomer | 0.434 |
| 304 | Mixture of Diastereomers | 0.021 |
| 305 | Mixture of Diastereomers | 0.025 |
| 306 | Single Stereoisomer | 0.129 |
| 307 | Single Stereoisomer | 0.095 |
| 308 | Single Stereoisomer | 0.825 |
| 309 | Single Stereoisomer | 0.408 |
| 310 | Single Stereoisomer | 0.365 |
| 311 | Single Stereoisomer | 0.590 |
| 312 | Single Stereoisomer | 0.119 |
| 313 | Single Stereoisomer | 0.105 |
| 314 | Single Stereoisomer | 0.082 |
| 315 | Mixture of Diastereomers | 0.014 |
| 316 | Mixture of Diastereomers | 0.044 |
| 317 | Mixture of Diastereomers | 0.225 |
| 318 | Single Stereoisomer | 0.010 |
| 319 | Single Stereoisomer | 0.197 |
| 320 | Single Stereoisomer | 0.093 |
| 321 | Single Stereoisomer | 0.246 |
| 322 | Single Stereoisomer | 0.012 |
| 323 | Single Stereoisomer | 0.035 |
| 324 | Single Stereoisomer | 0.084 |
| 325 | Mixture of Diastereomers | 0.162 |
| 326 | Mixture of Diastereomers | 0.076 |
| 327 | Single Stereoisomer | 0.035 |
| 328 | Mixture of Diastereomers | 0.054 |
| 329 | Mixture of Diastereomers | 0.037 |
| 330 | Single Stereoisomer | 0.041 |
| 331 | Single Stereoisomer | 0.148 |
| 332 | Single Stereoisomer | 0.168 |
| 333 | Single Stereoisomer | 0.198 |
| 334 | Single Stereoisomer | 0.739 |
| 335 | Single Stereoisomer | 0.002 |
| 336 | Mixture of Diastereomers | 0.368 |
| 337 | Single Stereoisomer | 0.021 |
| 338 | Single Stereoisomer | 0.009 |

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. Compounds of Formula (I): 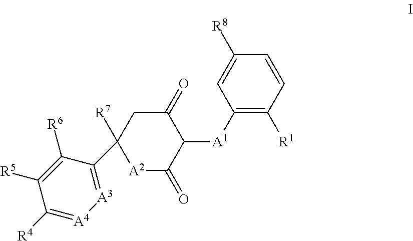
and stereoisomers, tautomers, and pharmaceutically acceptable salts thereof, wherein:
A¹ is O, CH₂ or S;
A² is NH or N—C₁-C₃-alkyl;
A³ is N or CR²;
A⁴ is N or CR³, provided that A³ and A⁴ are not N at the same time;
R¹ is Cl, NO₂, or CN;
R² and R⁶ are independently selected from the group consisting of H, halo, hydroxy, C₁-C₆-hydroxyalkyl, and NH₂;
R³ and R⁵ are independently selected from the group consisting of:
H;
hydroxy;
halo;
—C₁-C₆-alkyl-R^{f};
—C₁-C₆-alkenyl-R^{f};
—C₁-C₆-alkoxy-R^{c};
—NR^{a}R^{b};
—NR^{a}—(C₁-C₆-alkyl)-R^{d};
—NR^{a}—S(O)₂-(4 to 10 membered heterocycloalkyl);
—NR^{a}—(C₃-C₈-cycloalkyl), which cycloalkyl is unsubstituted or substituted by C₁-C₆-alkyl or a C₁-C₃-alkylene bridge;
—NR^{a}-aryl, which aryl is unsubstituted or substituted by one or more substituent(s) selected from the group consisting of:
halo, hydroxy, —NH₂, C₁-C₆-alkyl, C₁-C₆-alkoxy, C₁-C₆-haloalkyl, C₁-C₆-hydroxyalkyl, C₁-C₆-haloalkoxy and C₃-C₈-cycloalkyl;
—NR^{a}-(4 to 10 membered heterocycloalkyl), which heterocycloalkyl is unsubstituted or substituted by one or more substituent(s) selected from the group consisting of: C₁-C₆-alkyl, C₁-C₆-hydroxyalkyl, or —CO-alkyl;
—NR^{a}-(5 or 6 membered heteroaryl), which heteroaryl is unsubstituted or substituted by one or more substituent(s) selected from the group consisting of: halo, —NR^{a}R^{b} and C₁-C₆-alkyl;
—NR^{a}(CO)—C₁-C₆-alkyl;
—NR^{a}(CO)-aryl;
—NR^{a}(CO)-(5 or 6 membered heteroaryl);
—NR^{a}(CO)O—C₁-C₆-alkyl;
—S-(alkyl)ₙ-R^{h};
—S(O)₂-aryl, which aryl is unsubstituted or substituted by one or more halo;
—C(O)—R^{e};
—C(O)NR^{a}—(C₁-C₆-alkyl)ₙ-R^{g};
—C(O)NR^{a}—C₁-C₆-alkoxy;
—O—C₃-C₈-cycloalkyl, which cycloalkyl is unsubstituted or substituted by one or more substituent(s) selected from the group consisting of: halo or hydroxy, C₁-C₆-alkyl, C₁-C₆-alkoxy, C₁-C₆-haloalkoxy, C₁-C₆-alkoxyaryl, C₁-C₆-haloalkyl, C₁-C₆-hydroxyalkyl, NR^{a}R^{b}, aryl, C₁-C₆-akyl-aryl, 5 or 6 membered heteroaryl, and —(C₁-C₆-alkyl)-(C₁-C₆-alkoxy);
—O-aryl, which aryl is unsubstituted or substituted by one or more substituent(s) selected from the group consisting of:
halo, C₁-C₆-alkyl, C₁-C₆-alkoxy, C₁-C₆-alkyl-C₁-C₆-alkoxy, C₁-C₆-haloalkyl, C₁-C₆-haloalkoxy, C₁-C₆-hydroxyalkyl, —S—C₁-C₆-akyl, —C₁-C₆-alkyl-C₃-C₈-cycloalkyl, C₁-C₆-alkoxy-C₃-C₈-cycloalkyl, C₁-C₆-alkyl-(4 to 10 membered heterocycloalkyl), C₁-C₆-alkyl-(5 or 6 membered heterocycloalkyl), or 5 or 6 membered heteroaryl unsubstituted or substituted by one or more substituent(s) selected from the group consisting of: C₁-C₆-alkyl, —(C₁-C₆-alkyl)-(C₁-C₆-alkoxy), C₁-C₆-haloalkoxy and a C₁-C₆-alkylene bridge;
—O-(4 to 10 membered heterocycloalkyl), which heterocycloalkyl is unsubstituted or substituted by one or more substituent(s) selected from the group consisting of:
halo, hydroxy, C₁-C₆-alkyl, C₁-C₆-hydroxyalkyl and —C(O)—C₁-C₆-alkyl;
—O-(5 to 10 membered heteroaryl), which heteroaryl is unsubstituted or substituted by halo, C₁-C₆-alkyl, C₁-C₆-hydroxyalkyl, or —NR^{a}(CO)—C₁-C₆-akyl;
C₃-C₈-cycloalkyl, which cycloalkyl may be fused to a phenyl;
aryl unsubstituted or substituted by one or more substituent(s) selected from the group consisting of:
halo, hydroxy, —C(O)OH, C₁-C₆-hydroxyalkyl, C₁-C₆-alkoxy, —S(O)₂—NH(alkyl) and —S(O)₂—N(alkyl)₂;
4 to 10 membered heterocycloalkyl unsubstituted or substituted by one or more substituent(s) selected from the group consisting of:
halo, C₁-C₆-alkyl, —C(O)—C₃-C₈-cycloalkyl, oxo and 5 or 6 membered heterocycloalkyl;
5 to 10 membered heteroaryl unsubstituted or substituted by one or more substituent(s) selected from the group consisting of:
hydroxy, —NR^{a}R^{b}, C₁-C₆-alkyl, C₁-C₆-hydroxyalkyl, and 4 to 10 membered heterocycloalkyl;
R⁴ is:
H,
cyano,
halo,
hydroxy,
NR^{a}R^{b},
C₁-C₆-alkyl,
C₁-C₆-haloalkyl,
C₁-C₆-hydroxyalkyl,
C₁-C₆-alkoxy unsubstituted or substituted by hydroxy, C₁-C₆-alkoxy or NR^{a}R^{b},
—(C₁-C₆-alkyl)ₙ-(C₃-C₈-cycloalkyl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl, —C(O)—C₁-C₆-alkyl, —C(O)—C₁-C₆-cycloalkyl; —C(O)-(5 or 6 membered heterocycloalkyl);
—(C₁-C₆-alkyl)ₙ-(C₃-C₈-cycloalkenyl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl, —C(O)—C₁-C₆-alkyl, —C(O)—C₁-C₆-cycloalkyl and —C(O)-(5 or 6 membered heterocycloalkyl);
—(C₁-C₆-alkyl)ₙ-(5 or 6 membered heteroaryl), unsubstituted or substituted by one or more substituent(s) selected from the group consisting of: halo, hydroxy, —NR^{a}R^{b}, C₁-C₆-alkyl, C₁-C₆-alkoxy, C₁-C₆-haloalkyl and —C(O)—C₁-C₆-alkyl, —C(O)—C₁-C₆-cycloalkyl and —C(O)-(5 or 6 membered heterocycloalkyl);
—(C₁-C₆-alkyl)ₙ-(4 to 10 membered heterocycloalkyl) unsubstituted or substituted by one or more substituent(s) selected from the group consisting of: halo, hydroxy, cyano, —NR^{a}R^{b}, C₁-C₆-alkyl, C₁-C₆-alkoxy, C₁-C₆-haloalkyl, C₁-C₆-hydroxyalkyl, —C(O)OH, a C₁-C₄-alkylene bridge, —C(O)—C₁-C₆-alkyl, —C(O)—C₃-C₈-cycloalkyl, —C(O)-aryl, —C(O)(4 to 10 membered heterocycloalkyl) and —C(O)-(5 or 6 membered heterocycloalkyl);
R⁷ is aryl, a 5 or 6 membered heterocycle or 5 or 6 membered heteroaryl which aryl, heterocycle or heteroaryl is unsubstituted or substituted by one or more substituent(s) selected from the group consisting of halo, C₁-C₆-alkyl, C₃-C₈-cycloalkyl, —O-aryl, —S-aryl, —NH-aryl, and —(C₁-C₆-alkyl)ₙ-aryl;
or R⁶ and R⁷ together with the carbon atoms to which they are attached form a 5 membered ring selected from a cycloalkyl or heterocycloalkyl having 5 ring members;
R⁸ is OH, —NR^{a}R^{b}, C₁-C₆-alkoxy or —C(O)O—C₁-C₆-alkyl;
or R² and R³ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;
or R³ and R⁴ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;
or R⁴ and R⁵ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;
or R⁵ and R⁶ together with the atoms to which they are attached form a naphthyl or 9 or 10 membered heteroaryl, each of which is unsubstituted or substituted by one or more substituent(s) selected from the group consisting of:
halo, hydroxy, —NR^{a}R^{b}, C₁-C₆-alkyl, C₁-C₆-alkoxy and C₁-C₆-haloalkyl;
R^{a} is H or C₁-C₆-alkyl;
R^{b} is H or C₁-C₆-alkyl;
R^{c} is H, hydroxy, halo, —NR^{a}R^{b}, C₁-C₆-alkoxy, C₁-C₆-alkenyl, 4 to 6 membered heterocycloalkyl unsubstituted or substituted by oxo or C₁-C₆-alkyl, 5 or 6 membered heteroaryl unsubstituted or substituted by C₁-C₆-alkyl, or C₃-C₈-cycloalkyl unsubstituted or substituted by one or more substituent(s) selected from the group consisting of:
halo, C₁-C₆-alkyl or C₁-C₆-hydroxyalkyl, aryl unsubstituted or substituted by halo, 4 to 9 membered heterocycloalkyl unsubstituted or substituted by oxo or C₁-C₆-alkyl, and 5 or 6 membered heteroaryl unsubstituted or substituted by C₁-C₆-alkyl;
R^{d} is H, hydroxy, C₁-C₆-alkyl, C₃-C₈-cycloalkyl or aryl unsubstituted or substituted by one or more substituent(s) selected from the group consisting of halo and —NR^{a}—S(O)₂—N(C₁-C₆-alkyl)₂;
R^{e} is C₁-C₆-alkyl, aryl, C₃-C₈-cycloalkyl, 5 to 9 membered heterocycloalkyl or 5 or 6 membered heteroaryl and wherein said aryl, C₃-C₈-cycloalkyl, 5 to 9 membered heterocycloalkyl or 5 or 6 membered heteroaryl is unsubstituted or substituted by one or more substituent(s) selected from the group consisting of: halo, C₁-C₆-alkoxy, C₁-C₆-alkyl and C₁-C₆-haloalkyl;
R^{f} is H, C₃-C₈-cycloalkyl, 4 to 10 membered heterocycloalkyl, aryl, or 5 or 6 membered heteroaryl, which cycloalkyl, heterocycloalkyl, aryl, or heteroaryl is unsubstituted or substituted by one or more substituent(s) selected from the group consisting of halo, C₁-C₆-haloalkyl, C₁-C₆-alkyl, C₁-C₆-alkoxy and C₁-C₆-hydroxyalkyl;
R^{g} is C₁-C₆-alkoxy, C₃-C₈-cycloalkyl, aryl, 5 or 6 membered heteroaryl, 5 to 9 membered heterocycloalkyl, wherein said aryl, C₃-C₈-cycloalkyl, 5 to 9 membered heterocycloalkyl or 5 or 6 membered heteroaryl is unsubstituted or substituted by one or more substituent(s) selected from the group consisting of halo, C₁-C₆-alkoxy and C₁-C₆-hydroxyalkyl;
R^{h} is aryl, 5 or 6 membered heteroaryl, 4 to 10 membered heterocycloalkyl, C₃-C₈-cycloalkyl, each of which is unsubstituted or substituted by halo;
n is 0 or 1.

2. The compounds of claim 1, wherein it has the following general Formula: 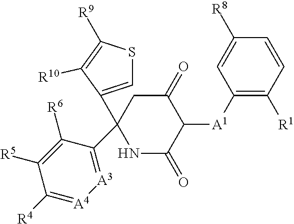
wherein A¹, A², A³, R¹, R³, R⁴, R⁵, R⁶, R⁸, R⁹ and R¹⁰ are as defined in claim 1.

3. The compounds of claim 1, wherein it has the following general Formula: 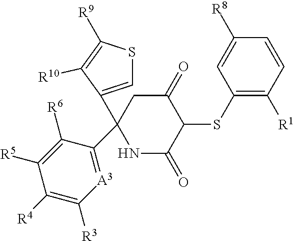
wherein A³, R¹, R³, R⁴, R⁵, R⁶, R⁸, R⁹ and R¹¹ are as defined in claim 1.

4. The compounds of claim 1, wherein it has the following general Formula: 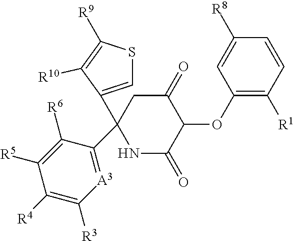
wherein A³, R¹, R³, R⁴, R⁵, R⁶, R⁸, R⁹ and R¹⁰ are as defined in claim 1.

5. The compound of claim 1, wherein A³ is NH.

6. The compound of claim 1, wherein A³ is CR², wherein R² is selected from the group consisting of H, halo, hydroxy, C₁-C₆-hydroxyalkyl, and NH.

7. The compound of claim 1, wherein R⁹ and R¹⁰ are H.

8. The compound of claim 1, wherein R¹ is Cl.

9. The compound of claim 1, wherein R³ is NH-phenyl or NH-pyridinyl, which phenyl or pyridinyl is substituted by halo.

10. The compound of claim 1, wherein R⁴, R⁵, R⁶ and R⁸ are H.

11. The compound of claim 1, wherein it is selected from the group consisting of the following compounds as racemates, single stereoisomers, tautomers and pharmaceutically acceptable salts thereof:
1-[4-[5-(2-chlorophenyl)sulfanyl-4,6-dioxo-2-(3-thienyl)-2-piperidyl]phenyl]piperidine-4-carbonitrile;
2-[[6-(6-bromo-2-pyridyl)-2,4-dioxo-6-(3-thienyl)-3-piperidyl]sulfanyl]benzonitrile;
3-(2-chloro-5-hydroxy-phenyl)sulfanyl-6-[4-(1-piperidyl)phenyl]-6-(3-thienyl)piperidine-2,4-dione;
3-(2-chlorophenoxy)-6-(4-morpholinophenyl)-6-(3-thienyl)piperidine-2,4-dione;
3-(2-chlorophenoxy)-6-[4-(1-piperidyl)phenyl]-6-(3-thienyl)piperidine-2,4-dione;
3-(2-chlorophenoxy)-6-[6-(2-cyclopropylethoxy)-2-pyridyl]-6-(3-thienyl)piperidine-2,4-dione;
3-(2-chlorophenoxy)-6-[6-(3,4-difluorophenoxy)-2-pyridyl]-6-(3-thienyl)piperidine-2,4-dione;
3-(2-chlorophenoxy)-6-[6-(4-fluoroanilino)-2-pyridyl]-6-(3-thienyl)piperidine-2,4-dione;
3-(2-chlorophenoxy)-6-[6-(4-fluorophenoxy)-2-pyridyl]-6-(3-thienyl)piperidine-2,4-dione;
3-(2-chlorophenyl)sulfanyl-1-methyl-6-(3-tetrahydropyran-4-yloxyphenyl)-6-(3-thienyl)piperidine-2,4-dione;
3-(2-chlorophenyl)sulfanyl-1-methyl-6-[3-(tetrahydropyran-4-ylamino)phenyl]-6-(3-thienyl)piperidine-2,4-dione;
3-(2-chlorophenyl)sulfanyl-6-(1H-indol-4-yl)-6-(3-thienyl)piperidine-2,4-dione;
3-(2-chlorophenyl)sulfanyl-6-(2-fluorophenyl)-1-methyl-6-(3-thienyl)piperidine-2,4-dione;
3-(2-chlorophenyl)sulfanyl-6-(2-hydroxy-4-morpholino-phenyl)-6-(3-thienyl)piperidine-2,4-dione;
3-(2-chlorophenyl)sulfanyl-6-(2-hydroxyphenyl)-6-(3-thienyl)piperidine-2,4-dione;
3-(2-chlorophenyl)sulfanyl-6-(2-naphthyl)-6-(3-thienyl)piperidine-2,4-dione;
3-(2-chlorophenyl)sulfanyl-6-(3-fluoro-4-morpholino-phenyl)-6-(3-thienyl)piperidine-2,4-dione;
3-(2-chlorophenyl)sulfanyl-6-(3-hydroxyphenyl)-6-(3-thienyl)piperidine-2,4-dione;
3-(2-chlorophenyl)sulfanyl-6-(3-tetrahydropyran-4-yloxyphenyl)-6-(3-thienyl)piperidine-2,4-dione;
3-(2-chlorophenyl)sulfanyl-6-(3-thienyl)-6-(4-thiomorpholinophenyl)piperidine-2,4-dione;
3-(2-chlorophenyl)sulfanyl-6-(3-thienyl)-6-[6-(2,2,2-trifluoro-1-methyl-ethoxy)-2-pyridyl]piperidine-2,4-dione;
3-(2-chlorophenyl)sulfanyl-6-(3-thienyl)-6-[6-(2,2,2-trifluoroethoxy)-2-pyridyl]piperidine-2,4-dione;
3-(2-chlorophenyl)sulfanyl-6-(3-thienyl)-6-[6-(4,4,4-trifluorobutoxy)-2-pyridyl]piperidine-2,4-dione;
3-(2-chlorophenyl)sulfanyl-6-(3-thienyl)-6-[6-[3-(trifluoromethyl)phenoxy]-2-pyridyl]piperidine-2,4-dione;
3-(2-chlorophenyl)sulfanyl-6-(3-thienyl)-6-[6-[4-(trifluoromethoxy)phenoxy]-2-pyridyl]piperidine-2,4-dione;
3-(2-chlorophenyl)sulfanyl-6-(3-thienyl)-6-[6-[4-(trifluoromethyl)cyclohexoxy]-2-pyridyl]piperidine-2,4-dione;
3-(2-chlorophenyl)sulfanyl-6-(3-thienyl)-6-[6-[4-(trifluoromethyl)phenoxy]-2-pyridyl]piperidine-2,4-dione;
3-(2-chlorophenyl)sulfanyl-6-(4-cyclohexylphenyl)-6-(3-thienyl)piperidine-2,4-dione;
3-(2-chlorophenyl)sulfanyl-6-(4-cyclopropylphenyl)-6-(3-thienyl)piperidine-2,4-dione;
3-(2-chlorophenyl)sulfanyl-6-(4-hydroxyphenyl)-6-(3-thienyl)piperidine-2,4-dione;
3-(2-chlorophenyl)sulfanyl-6-(4-morpholino-3-phenyl-phenyl)-6-(3-thienyl)piperidine-2,4-dione;
3-(2-chlorophenyl)sulfanyl-6-(4-morpholinophenyl)-6-(3-thienyl)piperidine-2,4-dione;
3-(2-chlorophenyl)sulfanyl-6-(4-morpholinophenyl)-6-(5-phenyl-3-thienyl)piperidine-2,4-dione;
3-(2-chlorophenyl)sulfanyl-6-(4-morpholinophenyl)-6-(6-tetrahydropyran-4-yloxy-2-pyridyl)piperidine-2,4-dione;
5-(2-chlorophenyl)sulfanyl-4-hydroxy-2-[6-(4-methoxycyclohexoxy)-2-pyridyl]-2-(3-thienyl)-1,3-dihydropyridin-6-one;
3-(2-chlorophenyl)sulfanyl-6-(4-morpholinophenyl)-6-thiazol-4-yl-piperidine-2,4-dione;
3-(2-chlorophenyl)sulfanyl-6-(4-piperazin-1-ylphenyl)-6-(3-thienyl)piperidine-2,4-dione;
3-(2-chlorophenyl)sulfanyl-6-(4-pyrrolidin-1-ylphenyl)-6-(3-thienyl)piperidine-2,4-dione;
3-(2-chlorophenyl)sulfanyl-6-(5-chloro-3-thienyl)-6-[6-(4-fluorophenoxy)-2-pyridyl]piperidine-2,4-dione;
3-(2-chlorophenyl)sulfanyl-6-(5-methyl-3-thienyl)-6-(4-morpholinophenyl)piperidine-2,4-dione;
3-(2-chlorophenyl)sulfanyl-6-(6-chroman-4-yloxy-2-pyridyl)-6-(3-thienyl)piperidine-2,4-dione;
3-(2-chlorophenyl)sulfanyl-6-(6-ethoxy-2-pyridyl)-6-(3-thienyl)piperidine-2,4-dione;
3-(2-chlorophenyl)sulfanyl-6-(6-indan-5-yloxy-2-pyridyl)-6-(3-thienyl)piperidine-2,4-dione;
3-(2-chlorophenyl)sulfanyl-6-(6-isobutoxy-2-pyridyl)-6-(3-thienyl)piperidine-2,4-dione;
3-(2-chlorophenyl)sulfanyl-6-(6-isopentyloxy-2-pyridyl)-6-(3-thienyl)piperidine-2,4-dione;
3-(2-chlorophenyl)sulfanyl-6-(6-isopropoxy-2-pyridyl)-6-(3-thienyl)piperidine-2,4-dione;
3-(2-chlorophenyl)sulfanyl-6-(6-isopropoxy-5-morpholino-2-pyridyl)-6-(3-thienyl)piperidine-2,4-dione;
3-(2-chlorophenyl)sulfanyl-6-(6-morpholino-3-pyridyl)-6-(3-thienyl)piperidine-2,4-dione;
3-(2-chlorophenyl)sulfanyl-6-(6-pent-2-enoxy-2-pyridyl)-6-(3-thienyl)piperidine-2,4-dione;
3-(2-chlorophenyl)sulfanyl-6-(6-phenoxy-2-pyridyl)-6-(3-thienyl)piperidine-2,4-dione;
3-(2-chlorophenyl)sulfanyl-6-(6-phenyl-2-pyridyl)-6-(3-thienyl)piperidine-2,4-dione;
3-(2-chlorophenyl)sulfanyl-6-(6-pyrimidin-5-yloxy-2-pyridyl)-6-(3-thienyl)piperidine-2,4-dione;
3-(2-chlorophenyl)sulfanyl-6-(6-tetrahydrofuran-3-yloxy-2-pyridyl)-6-(3-thienyl)piperidine-2,4-dione;
3-(2-chlorophenyl)sulfanyl-6-(6-tetralin-1-yloxy-2-pyridyl)-6-(3-thienyl)piperidine-2,4-dione;
3-(2-chlorophenyl)sulfanyl-6-[3-(4-fluoroanilino)phenyl]-1-methyl-6-(3-thienyl)piperidine-2,4-dione;
3-(2-chlorophenyl)sulfanyl-6-[3-(4-fluoroanilino)phenyl]-6-(3-thienyl)piperidine-2,4-dione;
3-(2-chlorophenyl)sulfanyl-6-[3-(4-fluoroanilino)phenyl]-6-phenyl-piperidine-2,4-dione;
3-(2-chlorophenyl)sulfanyl-6-[3-(4-fluoro-N-methyl-anilino)phenyl]-6-phenyl-piperidine-2,4-dione;
3-(2-chlorophenyl)sulfanyl-6-[3-(4-fluorophenoxy)phenyl]-6-(3-thienyl)piperidine-2,4-dione;
3-(2-chlorophenyl)sulfanyl-6-[3-(cyclohexylamino)phenyl]-6-(3-thienyl)piperidine-2,4-dione;
3-(2-chlorophenyl)sulfanyl-6-[3-(tetrahydropyran-4-ylamino)phenyl]-6-(3-thienyl)piperidine-2,4-dione;
3-(2-chlorophenyl)sulfanyl-6-[3-[(6-fluoro-5-methyl-3-pyridyl)amino]phenyl]-6-(3-thienyl)piperidine-2,4-dione;
3-(2-chlorophenyl)sulfanyl-6-[4-(1-piperidyl)phenyl]-6-(3-thienyl)piperidine-2,4-dione;
3-(2-chlorophenyl)sulfanyl-6-[4-(2,2-dimethylmorpholin-4-yl)phenyl]-6-(3-thienyl)piperidine-2,4-dione;
3-(2-chlorophenyl)sulfanyl-6-[4-(2,6-dimethylmorpholin-4-yl)phenyl]-6-(3-thienyl)piperidine-2,4-dione;
3-(2-chlorophenyl)sulfanyl-6-[4-(2-ethylmorpholin-4-yl)phenyl]-6-(3-thienyl)piperidine-2,4-dione;
3-(2-chlorophenyl)sulfanyl-6-[4-(2-hydroxyethoxy)phenyl]-6-(3-thienyl)piperidine-2,4-dione;
3-(2-chlorophenyl)sulfanyl-6-[4-(2-methoxyethoxy)phenyl]-6-(3-thienyl)piperidine-2,4-dione;
3-(2-chlorophenyl)sulfanyl-6-[4-(2-methylmorpholin-4-yl)phenyl]-6-(3-thienyl)piperidine-2,4-dione;
3-(2-chlorophenyl)sulfanyl-6-[4-(2-oxa-5-azabicyclo[2.2.1]heptan-5-yl)phenyl]-6-(3-thienyl)piperidine-2,4-dione;
3-(2-chlorophenyl)sulfanyl-6-[4-(2-oxa-6-azaspiro[3.3]heptan-6-yl)phenyl]-6-(3-thienyl)piperidine-2,4-dione;
3-(2-chlorophenyl)sulfanyl-6-[4-(2-oxa-7-azaspiro[3.5]nonan-7-yl)phenyl]-6-(3-thienyl)piperidine-2,4-dione;
3-(2-chlorophenyl)sulfanyl-6-[4-(3,3-difluoroazetidin-1-yl)phenyl]-6-(3-thienyl)piperidine-2,4-dione;
3-(2-chlorophenyl)sulfanyl-6-[4-(3,3-difluoropyrrolidin-1-yl)phenyl]-6-(3-thienyl)piperidine-2,4-dione;
3-(2-chlorophenyl)sulfanyl-6-[4-(3-fluoroazetidin-1-yl)phenyl]-6-(3-thienyl)piperidine-2,4-dione;
3-(2-chlorophenyl)sulfanyl-6-[4-(3-fluoropyrrolidin-1-yl)phenyl]-6-(3-thienyl)piperidine-2,4-dione;
3-(2-chlorophenyl)sulfanyl-6-[4-(3-hydroxypropoxy)phenyl]-6-(3-thienyl)piperidine-2,4-dione;
3-(2-chlorophenyl)sulfanyl-6-[4-(3-methoxypropoxy)phenyl]-6-(3-thienyl)piperidine-2,4-dione;
3-(2-chlorophenyl)sulfanyl-6-[4-(3-methoxypyrrolidin-1-yl)phenyl]-6-(3-thienyl)piperidine-2,4-dione;
3-(2-chlorophenyl)sulfanyl-6-[4-(4,4-difluoro-1-piperidyl)phenyl]-6-(3-thienyl)piperidine-2,4-dione;
3-(2-chlorophenyl)sulfanyl-6-[4-(4-fluoro-1-piperidyl)phenyl]-6-(3-thienyl)piperidine-2,4-dione;
3-(2-chlorophenyl)sulfanyl-6-[4-(4-methoxy-1-piperidyl)phenyl]-6-(3-thienyl)piperidine-2,4-dione;
3-(2-chlorophenyl)sulfanyl-6-[4-(8-oxa-3-azabicyclo[3.2.1]octan-3-yl)phenyl]-6-(3-thienyl)piperidine-2,4-dione;
3-(2-chlorophenyl)sulfanyl-6-[4-(cyclohexen-1-yl)phenyl]-6-(3-thienyl)piperidine-2,4-dione;
3-(2-chlorophenyl)sulfanyl-6-[4-(dimethylamino)phenyl]-6-(3-thienyl)piperidine-2,4-dione;
3-(2-chlorophenyl)sulfanyl-6-[4-(tetrahydropyran-4-yl amino)phenyl]-6-(3-thienyl)piperidine-2,4-dione;
3-(2-chlorophenyl)sulfanyl-6-[5-(4-fluoroanilino)-2-hydroxy-phenyl]-6-(3-thienyl)piperidine-2,4-dione;
3-(2-chlorophenyl)sulfanyl-6-[5-[(4-fluorophenyl)methyl]-3-thienyl]-6-(4-morpholinophenyl)piperidine-2,4-dione;
3-(2-chlorophenyl)sulfanyl-6-[6-(1,2,3,4-tetrahydroquinolin-8-yloxy)-2-pyridyl]-6-(3-thienyl)piperidine-2,4-dione;
3-(2-chlorophenyl)sulfanyl-6-[6-(1-cyclohexylethoxy)-2-pyridyl]-6-(3-thienyl)piperidine-2,4-dione;
3-(2-chlorophenyl)sulfanyl-6-[6-(1-cyclopropylethoxy)-2-pyridyl]-6-(3-thienyl)piperidine-2,4-dione;
3-(2-chlorophenyl)sulfanyl-6-[6-(1-cyclopropylethylamino)-2-pyridyl]-6-(3-thienyl)piperidine-2,4-dione;
3-(2-chlorophenyl)sulfanyl-6-[6-(1H-indazol-4-yloxy)-2-pyridyl]-6-(3-thienyl)piperidine-2,4-dione;
3-(2-chlorophenyl)sulfanyl-6-[6-(2,2-difluoroethoxy)-2-pyridyl]-6-(3-thienyl)piperidine-2,4-dione;
3-(2-chlorophenyl)sulfanyl-6-[6-(2,2-dimethylchroman-4-yl)oxy-2-pyridyl]-6-(3-thienyl)piperidine-2,4-dione;
3-(2-chlorophenyl)sulfanyl-6-[6-(2,2-dimethylpropoxy)-2-pyridyl]-6-(3-thienyl)piperidine-2,4-dione;
3-(2-chlorophenyl)sulfanyl-6-[6-(2,3-difluorophenoxy)-2-pyridyl]-6-(3-thienyl)piperidine-2,4-dione;
3-(2-chlorophenyl)sulfanyl-6-[6-(2,4-difluorophenoxy)-2-pyridyl]-6-(3-thienyl)piperidine-2,4-dione;
3-(2-chlorophenyl)sulfanyl-6-[6-(2-cyclobutylethoxy)-2-pyridyl]-6-(3-thienyl)piperidine-2,4-dione;
3-(2-chlorophenyl)sulfanyl-6-[6-(2-cyclohexylethoxy)-2-pyridyl]-6-(3-thienyl)piperidine-2,4-dione;
3-(2-chlorophenyl)sulfanyl-6-[6-(2-cyclohexylethylamino)-2-pyridyl]-6-(3-thienyl)piperidine-2,4-dione;
3-(2-chlorophenyl)sulfanyl-6-[6-(2-cyclopentylethoxy)-2-pyridyl]-6-(3-thienyl)piperidine-2,4-dione;
3-(2-chlorophenyl)sulfanyl-6-[6-(2-cyclopropyl-1-methyl-ethoxy)-2-pyridyl]-6-(3-thienyl)piperidine-2,4-dione;
3-(2-chlorophenyl)sulfanyl-6-[6-(2-cyclopropylethoxy)-2-pyridyl]-6-(3-thienyl)piperidine-2,4-dione;
3-(2-chlorophenyl)sulfanyl-6-[6-(2-cyclopropylethylamino)-2-pyridyl]-6-(3-thienyl)piperidine-2,4-dione;
3-(2-chlorophenyl)sulfanyl-6-[6-(2-cyclopropylpropoxy)-2-pyridyl]-6-(3-thienyl)piperidine-2,4-dione;
3-(2-chlorophenyl)sulfanyl-6-[6-(2-ethoxy-1-methyl-ethoxy)-2-pyridyl]-6-(3-thienyl)piperidine-2,4-dione;
3-(2-chlorophenyl)sulfanyl-6-[6-(2-ethoxyethoxy)-2-pyridyl]-6-(3-thienyl)piperidine-2,4-dione;
3-(2-chlorophenyl)sulfanyl-6-[6-(2-fluorophenoxy)-2-pyridyl]-6-(3-thienyl)piperidine-2,4-dione;
3-(2-chlorophenyl)sulfanyl-6-[6-(2-methoxy-1-methyl-ethoxy)-2-pyridyl]-6-(3-thienyl)piperidine-2,4-dione;
3-(2-chlorophenyl)sulfanyl-6-[6-(2-methoxyphenoxy)-2-pyridyl]-6-(3-thienyl)piperidine-2,4-dione;
3-(2-chlorophenyl)sulfanyl-6-[6-(2-methylbutoxy)-2-pyridyl]-6-(3-thienyl)piperidine-2,4-dione;
3-(2-chlorophenyl)sulfanyl-6-[6-(2-morpholino-4-pyridyl)-2-pyridyl]-6-(3-thienyl)piperidine-2,4-dione;
3-(2-chlorophenyl)sulfanyl-6-[6-(2-pyridyloxy)-2-pyridyl]-6-(3-thienyl)piperidine-2,4-dione;
3-(2-chlorophenyl)sulfanyl-6-[6-(3,4-difluoroanilino)-2-pyridyl]-6-(3-thienyl)piperidine-2,4-dione;
3-(2-chlorophenyl)sulfanyl-6-[6-(3,4-difluorophenoxy)-2-pyridyl]-6-(3-thienyl)piperidine-2,4-dione;
3-(2-chlorophenyl)sulfanyl-6-[6-(3,4-difluorophenoxy)-2-pyridyl]-6-(4-morpholinophenyl)piperidine-2,4-dione;
3-(2-chlorophenyl)sulfanyl-6-[6-(3,4-difluorophenoxy)-2-pyridyl]-6-[4-(1-piperidyl)phenyl]piperidine-2,4-dione;
3-(2-chlorophenyl)sulfanyl-6-[6-(3,5-difluorophenoxy)-2-pyridyl]-6-(3-thienyl)piperidine-2,4-dione;
3-(2-chlorophenyl)sulfanyl-6-[6-(3-fluoro-4-methoxy-phenoxy)-2-pyridyl]-6-(3-thienyl)piperidine-2,4-dione;
3-(2-chlorophenyl)sulfanyl-6-[6-(3-fluorophenoxy)-2-pyridyl]-6-(3-thienyl)piperidine-2,4-dione;
3-(2-chlorophenyl)sulfanyl-6-[6-(3-hydroxy-3-methyl-butoxy)-2-pyridyl]-6-(3-thienyl)piperidine-2,4-dione;
3-(2-chlorophenyl)sulfanyl-6-[6-(3-hydroxycyclopentoxy)-2-pyridyl]-6-(3-thienyl)piperidine-2,4-dione;
3-(2-chlorophenyl)sulfanyl-6-[6-(3-methoxy-3-methyl-butoxy)-2-pyridyl]-6-(3-thienyl)piperidine-2,4-dione;
3-(2-chlorophenyl)sulfanyl-6-[6-(3-methoxy-N-methyl-anilino)-2-pyridyl]-6-(3-thienyl)piperidine-2,4-dione;
3-(2-chlorophenyl)sulfanyl-6-[6-(3-methoxyphenoxy)-2-pyridyl]-6-(3-thienyl)piperidine-2,4-dione;
3-(2-chlorophenyl)sulfanyl-6-[6-(3-methoxypropoxy)-2-pyridyl]-6-(3-thienyl)piperidine-2,4-dione;
3-(2-chlorophenyl)sulfanyl-6-[6-(3-pyridyloxy)-2-pyridyl]-6-(3-thienyl)piperidine-2,4-dione;
3-(2-chlorophenyl)sulfanyl-6-[6-(3-tetrahydropyran-4-ylazetidin-1-yl)-2-pyridyl]-6-(3-thienyl)piperidine-2,4-dione;
3-(2-chlorophenyl)sulfanyl-6-[6-(4,4-difluorocyclohexoxy)-2-pyridyl]-6-(3-thienyl)piperidine-2,4-dione;
3-(2-chlorophenyl)sulfanyl-6-[6-(4-cyclopropyl-2-fluoro-anilino)-2-pyridyl]-6-(3-thienyl)piperidine-2,4-dione;
3-(2-chlorophenyl)sulfanyl-6-[6-(4-fluoro-2-isopropyl-phenoxy)-2-pyridyl]-6-(3-thienyl)piperidine-2,4-dione;
3-(2-chlorophenyl)sulfanyl-6-[6-(4-fluoro-2-methoxy-phenoxy)-2-pyridyl]-6-(3-thienyl)piperidine-2,4-dione;
(6S)-3-(2-chlorophenyl)sulfanyl-6-[6-(4-fluoro-2-methoxy-phenoxy)-2-pyridyl]-6-(3-thienyl)piperidine-2,4-dione;
3-(2-chlorophenyl)sulfanyl-6-[6-(4-fluoro-2-tetrahydropyran-4-yl-phenoxy)-2-pyridyl]-6-(3-thienyl)piperidine-2,4-dione;
3-(2-chlorophenyl)sulfanyl-6-[6-(4-fluoro-3-methoxy-phenyl)-2-pyridyl]-6-(3-thienyl)piperidine-2,4-dione;
3-(2-chlorophenyl)sulfanyl-6-[6-(4-fluoro-3-methyl-phenoxy)-2-pyridyl]-6-(3-thienyl)piperidine-2,4-dione;
3-(2-chlorophenyl)sulfanyl-6-[6-(4-fluoroanilino)-2-pyridyl]-1-methyl-6-(3-thienyl)piperidine-2,4-dione;
3-(2-chlorophenyl)sulfanyl-6-[6-(4-fluoroanilino)-2-pyridyl]-6-(3-thienyl)piperidine-2,4-dione;
3-(2-chlorophenyl)sulfanyl-6-[6-(4-fluoroanilino)-2-pyridyl]-6-(4-morpholinophenyl)piperidine-2,4-dione;
3-(2-chlorophenyl)sulfanyl-6-[6-(4-fluoroanilino)-5-morpholino-2-pyridyl]-6-(3-thienyl)piperidine-2,4-dione;
3-(2-chlorophenyl)sulfanyl-6-[6-(4-fluorobenzoyl)-2-pyridyl]-6-(3-thienyl)piperidine-2,4-dione;
3-(2-chlorophenyl)sulfanyl-6-[6-(4-fluoro-N-methyl-anilino)-2-pyridyl]-6-(3-thienyl)piperidine-2,4-dione;
3-(2-chlorophenyl)sulfanyl-6-[6-(4-fluorophenoxy)-2-pyridyl]-1-methyl-6-(3-thienyl)piperidine-2,4-dione;
3-(2-chlorophenyl)sulfanyl-6-[6-(4-fluorophenoxy)-2-pyridyl]-6-(1H-pyrazol-3-yl)piperidine-2,4-dione;
3-(2-chlorophenyl)sulfanyl-6-[6-(4-fluorophenoxy)-2-pyridyl]-6-(2-hydroxyphenyl)piperidine-2,4-dione;
3-(2-chlorophenyl)sulfanyl-6-[6-(4-fluorophenoxy)-2-pyridyl]-6-(3-thienyl)piperidine-2,4-dione;
3-(2-chlorophenyl)sulfanyl-6-[6-(4-fluorophenoxy)-2-pyridyl]-6-(4-morpholinophenyl)piperidine-2,4-dione;
3-(2-chlorophenyl)sulfanyl-6-[6-(4-fluorophenoxy)-5-morpholino-2-pyridyl]-6-(3-thienyl)piperidine-2,4-dione;
3-(2-chlorophenoxy)-6-[6-(4-fluoroanilino)-2-pyridyl]-6-(3-thienyl)piperidine-2,4-dione;
3-(2-chlorophenyl)sulfanyl-6-[6-(4-fluorophenyl)-2-pyridyl]-6-(3-thienyl)piperidine-2,4-dione;
3-(2-chlorophenyl)sulfanyl-6-[6-(4-fluorophenyl)sulfanyl-2-pyridyl]-6-(3-thienyl)piperidine-2,4-dione;
3-(2-chlorophenyl)sulfanyl-6-(3-thienyl)-6-[6-[3-(trifluoromethyl)phenoxy]-2-pyridyl]piperidine-2,4-dione;
3-(2-chlorophenyl)sulfanyl-6-[6-(cyclohexoxy)-2-pyridyl]-6-(3-thienyl)piperidine-2,4-dione;
3-(2-chlorophenyl)sulfanyl-6-[6-(4-hydroxy-4-methyl-pentoxy)-2-pyridyl]-6-(3-thienyl)piperidine-2,4-dione;
3-(2-chlorophenyl)sulfanyl-6-[6-(4-iodophenoxy)-2-pyridyl]-6-(3-thienyl)piperidine-2,4-dione;
3-(2-chlorophenyl)sulfanyl-6-[6-(4-methoxycyclohexoxy)-2-pyridyl]-6-(3-thienyl)piperidine-2,4-dione;
3-(2-chlorophenyl)sulfanyl-6-[6-(4-methoxy-N-methyl-anilino)-2-pyridyl]-6-(3-thienyl)piperidine-2,4-dione;
3-(2-chlorophenyl)sulfanyl-6-[6-(4-methoxyphenoxy)-2-pyridyl]-6-(3-thienyl)piperidine-2,4-dione;
3-(2-chlorophenyl)sulfanyl-6-[6-(4-methyl sulfanylphenoxy)-2-pyridyl]-6-(3-thienyl)piperidine-2,4-dione;
3-(2-chlorophenyl)sulfanyl-6-[6-(4-pyridyl)-2-pyridyl]-6-(3-thienyl)piperidine-2,4-dione;
3-(2-chlorophenyl)sulfanyl-6-[6-(4-pyridylmethoxy)-2-pyridyl]-6-(3-thienyl)piperidine-2,4-dione;
3-(2-chlorophenyl)sulfanyl-6-[6-(5-fluorotetralin-1-yl)oxy-2-pyridyl]-6-(3-thienyl)piperidine-2,4-dione;
3-(2-chlorophenyl)sulfanyl-6-[6-(5-isoquinolyloxy)-2-pyridyl]-6-(3-thienyl)piperidine-2,4-dione;
3-(2-chlorophenyl)sulfanyl-6-[6-(5-quinolyloxy)-2-pyridyl]-6-(3-thienyl)piperidine-2,4-dione;
3-(2-chlorophenyl)sulfanyl-6-[6-(6-fluorotetralin-1-yl)oxy-2-pyridyl]-6-(3-thienyl)piperidine-2,4-dione;
3-(2-chlorophenyl)sulfanyl-6-[6-(6-quinolyloxy)-2-pyridyl]-6-(3-thienyl)piperidine-2,4-dione;
3-(2-chlorophenyl)sulfanyl-6-[6-(7-fluorotetralin-1-yl)oxy-2-pyridyl]-6-(3-thienyl)piperidine-2,4-dione;
3-(2-chlorophenyl)sulfanyl-6-[6-(8-fluorochroman-4-yl)oxy-2-pyridyl]-6-(3-thienyl)piperidine-2,4-dione;
3-(2-chlorophenyl)sulfanyl-6-[6-(8-hydroxy-3,4-dihydro-2H-quinolin-1-yl)-2-pyridyl]-6-(3-thienyl)piperidine-2,4-dione;
3-(2-chlorophenyl)sulfanyl-6-[6-(8-isoquinolyloxy)-2-pyridyl]-6-(3-thienyl)piperidine-2,4-dione;
3-(2-chlorophenyl)sulfanyl-6-[6-(8-quinolyloxy)-2-pyridyl]-6-(3-thienyl)piperidine-2,4-dione;
3-(2-chlorophenyl)sulfanyl-6-[6-(cyclobutoxy)-2-pyridyl]-6-(3-thienyl)piperidine-2,4-dione;
3-(2-chlorophenyl)sulfanyl-6-[6-(cyclobutylmethoxy)-2-pyridyl]-6-(3-thienyl)piperidine-2,4-dione;
3-(2-chlorophenyl)sulfanyl-6-[6-(cycloheptoxy)-2-pyridyl]-6-(3-thienyl)piperidine-2,4-dione;
3-(2-chlorophenyl)sulfanyl-6-[6-(cyclohexoxy)-2-pyridyl]-6-(3-thienyl)piperidine-2,4-dione;
3-(2-chlorophenyl)sulfanyl-6-[6-(cyclohexoxy)-2-pyridyl]-6-(4-morpholinophenyl)piperidine-2,4-dione;
3-(2-chlorophenyl)sulfanyl-6-[6-(cyclohexoxy)-2-pyridyl]-6-[4-(1-piperidyl)phenyl]piperidine-2,4-dione;
3-(2-chlorophenyl)sulfanyl-6-[6-(cyclohexylamino)-2-pyridyl]-6-(3-thienyl)piperidine-2,4-dione;
3-(2-chlorophenyl)sulfanyl-6-[6-(cyclohexylmethoxy)-2-pyridyl]-6-(3-thienyl)piperidine-2,4-dione;
3-(2-chlorophenyl)sulfanyl-6-[6-(cyclopentoxy)-2-pyridyl]-6-(3-thienyl)piperidine-2,4-dione;
3-(2-chlorophenyl)sulfanyl-6-[6-(cyclopentylamino)-2-pyridyl]-6-(3-thienyl)piperidine-2,4-dione;
3-(2-chlorophenyl)sulfanyl-6-[6-(cyclopentylmethoxy)-2-pyridyl]-6-(3-thienyl)piperidine-2,4-dione;
3-(2-chlorophenyl)sulfanyl-6-[6-(cyclopropylmethoxy)-2-pyridyl]-6-(3-thienyl)piperidine-2,4-dione;
3-(2-chlorophenyl)sulfanyl-6-[6-(dimethylamino)-2-pyridyl]-6-(3-thienyl)piperidine-2,4-dione;
3-(2-chlorophenyl)sulfanyl-6-[6-(N-ethyl-4-fluoro-anilino)-2-pyridyl]-6-(3-thienyl)piperidine-2,4-dione;
3-(2-chlorophenyl)sulfanyl-6-[6-(oxetan-3-ylmethoxy)-2-pyridyl]-6-(3-thienyl)piperidine-2,4-dione;
3-(2-chlorophenyl)sulfanyl-6-[6-(tetrahydrofuran-2-ylmethoxy)-2-pyridyl]-6-(3-thienyl)piperidine-2,4-dione;
3-(2-chlorophenyl)sulfanyl-6-[6-(tetrahydrofuran-3-yl amino)-2-pyridyl]-6-(3-thienyl)piperidine-2,4-dione;
3-(2-chlorophenyl)sulfanyl-6-[6-(tetrahydropyran-4-yl amino)-2-pyridyl]-6-(3-thienyl)piperidine-2,4-dione;
3-(2-chlorophenyl)sulfanyl-6-[6-(tetrahydropyran-4-ylmethoxy)-2-pyridyl]-6-(3-thienyl)piperidine-2,4-dione;
3-(2-chlorophenyl)sulfanyl-6-[6-(tetrahydropyran-4-ylmethyl)-2-pyridyl]-6-(3-thienyl)piperidine-2,4-dione;
3-(2-chlorophenyl)sulfanyl-6-[6-(thiazol-2-ylamino)-2-pyridyl]-6-(3-thienyl)piperidine-2,4-dione;
3-(2-chlorophenyl)sulfanyl-6-[6-[(1,5-dimethylpyrazol-3-yl)amino]-2-pyridyl]-6-(3-thienyl)piperidine-2,4-dione;
3-(2-chlorophenyl)sulfanyl-6-[6-[(1-methyl-1,2,4-triazol-3-yl)amino]-2-pyridyl]-6-(3-thienyl)piperidine-2,4-dione;
3-(2-chlorophenyl)sulfanyl-6-[6-[(1-methylcyclopropyl)methoxy]-2-pyridyl]-6-(3-thienyl)piperidine-2,4-dione;
3-(2-chlorophenyl)sulfanyl-6-[6-[(1-methylimidazol-2-yl)amino]-2-pyridyl]-6-(3-thienyl)piperidine-2,4-dione;
3-(2-chlorophenyl)sulfanyl-6-[6-[(1-methylimidazol-2-yl)methoxy]-2-pyridyl]-6-(3-thienyl)piperidine-2,4-dione;
3-(2-chlorophenyl)sulfanyl-6-[6-[(1-methylpyrazol-3-yl)amino]-2-pyridyl]-6-(3-thienyl)piperidine-2,4-dione;
3-(2-chlorophenyl)sulfanyl-6-[6-[(2,4-difluorophenyl)methyl]-2-pyridyl]-6-(3-thienyl)piperidine-2,4-dione;
3-(2-chlorophenyl)sulfanyl-6-[6-[(2,5-dimethylpyrazol-3-yl)amino]-2-pyridyl]-6-(3-thienyl)piperidine-2,4-dione;
3-(2-chlorophenyl)sulfanyl-6-[6-[(2-methylcyclopropyl)methoxy]-2-pyridyl]-6-(3-thienyl)piperidine-2,4-dione;
3-(2-chlorophenyl)sulfanyl-6-[6-[(2-methylpyrazol-3-yl)amino]-2-pyridyl]-6-(3-thienyl)piperidine-2,4-dione;
3-(2-chlorophenyl)sulfanyl-6-[6-[(3,3-difluorocyclobutyl)methoxy]-2-pyridyl]-6-(3-thienyl)piperidine-2,4-dione;
3-(2-chlorophenyl)sulfanyl-6-[6-[(3,4-difluorophenyl)methyl]-2-pyridyl]-6-(3-thienyl)piperidine-2,4-dione;
3-(2-chlorophenyl)sulfanyl-6-[6-[(3,5-difluorophenyl)methyl]-2-pyridyl]-6-(3-thienyl)piperidine-2,4-dione;
3-(2-chlorophenyl)sulfanyl-6-[6-[(3-ethyloxetan-3-yl)methoxy]-2-pyridyl]-6-(3-thienyl)piperidine-2,4-dione;
3-(2-chlorophenyl)sulfanyl-6-[6-[(3-fluoro-5-methoxy-phenyl)methyl]-2-pyridyl]-6-(3-thienyl)piperidine-2,4-dione;
3-(2-chlorophenyl)sulfanyl-6-[6-[(3-fluorophenyl)methyl]-2-pyridyl]-6-(3-thienyl)piperidine-2,4-dione;
3-(2-chlorophenyl)sulfanyl-6-[6-[(4-fluoro-3-methoxy-phenyl)methyl]-2-pyridyl]-6-(3-thienyl)piperidine-2,4-dione;
3-(2-chlorophenyl)sulfanyl-6-[6-[(4-fluorophenyl)methoxy]-2-pyridyl]-6-(3-thienyl)piperidine-2,4-dione;
3-(2-chlorophenyl)sulfanyl-6-[6-[(4-fluorophenyl)methyl]-2-pyridyl]-6-(3-thienyl)piperidine-2,4-dione;
3-(2-chlorophenyl)sulfanyl-6-[6-[(4-fluorophenyl)methylamino]-2-pyridyl]-6-(3-thienyl)piperidine-2,4-dione;
3-(2-chlorophenyl)sulfanyl-6-[6-[(4-methylthiazol-2-yl)amino]-2-pyridyl]-6-(3-thienyl)piperidine-2,4-dione;
3-(2-chlorophenyl)sulfanyl-6-[6-[(5-fluoro-3-pyridyl)oxy]-2-pyridyl]-6-(3-thienyl)piperidine-2,4-dione;
3-(2-chlorophenyl)sulfanyl-6-[6-[(5-fluoro-8-quinolyl)oxy]-2-pyridyl]-6-(3-thienyl)piperidine-2,4-dione;
3-(2-chlorophenyl)sulfanyl-6-[6-[(5-methyl-1H-imidazol-2-yl)amino]-2-pyridyl]-6-(3-thienyl)piperidine-2,4-dione;
3-(2-chlorophenyl)sulfanyl-6-[6-[(5-methylthiazol-2-yl)amino]-2-pyridyl]-6-(3-thienyl)piperidine-2,4-dione;
3-(2-chlorophenyl)sulfanyl-6-[6-[(5-oxotetrahydrofuran-2-yl)methoxy]-2-pyridyl]-6-(3-thienyl)piperidine-2,4-dione;
3-(2-chlorophenyl)sulfanyl-6-[6-[(6-fluoro-3-pyridyl)amino]-2-pyridyl]-6-(3-thienyl)piperidine-2,4-dione;
3-(2-chlorophenyl)sulfanyl-6-[6-[(6-fluoro-5-methyl-3-pyridyl)amino]-2-pyridyl]-6-(3-thienyl)piperidine-2,4-dione;
3-(2-chlorophenyl)sulfanyl-6-[6-[[3-(hydroxymethyl)phenyl]methyl]-2-pyridyl]-6-(3-thienyl)piperidine-2,4-dione;
3-(2-chlorophenyl)sulfanyl-6-[6-[[4-(hydroxymethyl)cyclohexyl]methoxy]-2-pyridyl]-6-(3-thienyl)piperidine-2,4-dione;
3-(2-chlorophenyl)sulfanyl-6-[6-[1-(3,4-difluorophenyl)ethoxy]-2-pyridyl]-6-(3-thienyl)piperidine-2,4-dione;
3-(2-chlorophenyl)sulfanyl-6-[6-[1-(3-fluorophenyl)ethoxy]-2-pyridyl]-6-(3-thienyl)piperidine-2,4-dione;
3-(2-chlorophenyl)sulfanyl-6-[6-[1-(4-fluorophenyl)ethoxy]-2-pyridyl]-6-(3-thienyl)piperidine-2,4-dione;
3-(2-chlorophenyl)sulfanyl-6-[6-[1-(4-fluorophenyl)ethylamino]-2-pyridyl]-6-(3-thienyl)piperidine-2,4-dione;
3-(2-chlorophenyl)sulfanyl-6-[6-[1-(4-fluorophenyl)propoxy]-2-pyridyl]-6-(3-thienyl)piperidine-2,4-dione;
3-(2-chlorophenyl)sulfanyl-6-[6-[1-(4-fluorophenyl)propylamino]-2-pyridyl]-6-(3-thienyl)piperidine-2,4-dione;
3-(2-chlorophenyl)sulfanyl-6-[6-[2-(1H-pyrazol-4-yl)phenoxy]-2-pyridyl]-6-(3-thienyl)piperidine-2,4-dione;
3-(2-chlorophenyl)sulfanyl-6-[6-[2-(1-methylcyclopropyl)ethoxy]-2-pyridyl]-6-(3-thienyl)piperidine-2,4-dione;
3-(2-chlorophenyl)sulfanyl-6-[6-[2-(2,2-difluorocyclopropyl)ethoxy]-2-pyridyl]-6-(3-thienyl)piperidine-2,4-dione;
3-(2-chlorophenyl)sulfanyl-6-[6-[2-(2,2-dimethyl-1,3-dioxolan-4-yl)ethoxy]-2-pyridyl]-6-(3-thienyl)piperidine-2,4-dione;
3-(2-chlorophenyl)sulfanyl-6-[6-[2-(2-oxopyrrolidin-1-yl)ethoxy]-2-pyridyl]-6-(3-thienyl)piperidine-2,4-dione;
3-(2-chlorophenyl)sulfanyl-6-[6-[2-(3-methyltriazol-4-yl)phenoxy]-2-pyridyl]-6-(3-thienyl)piperidine-2,4-dione;
3-(2-chlorophenyl)sulfanyl-6-[6-[2-(4-fluorophenyl)ethyl]-2-pyridyl]-6-(3-thienyl)piperidine-2,4-dione;
3-(2-chlorophenyl)sulfanyl-6-[6-[2-(cyclopropylmethoxy)-4-fluoro-phenoxy]-2-pyridyl]-6-(3-thienyl)piperidine-2,4-dione;
3-(2-chlorophenyl)sulfanyl-6-[6-[2-(cyclopropylmethyl)-4-fluoro-phenoxy]-2-pyridyl]-6-(3-thienyl)piperidine-2,4-dione;
3-(2-chlorophenyl)sulfanyl-6-[6-[2-(methoxymethyl)phenoxy]-2-pyridyl]-6-(3-thienyl)piperidine-2,4-dione;
3-(2-chlorophenyl)sulfanyl-6-[6-[2-(oxetan-3-yl)ethoxy]-2-pyridyl]-6-(3-thienyl)piperidine-2,4-dione;
3-(2-chlorophenyl)sulfanyl-6-[6-[3-(1-hydroxyethyl)anilino]-2-pyridyl]-6-(3-thienyl)piperidine-2,4-dione;
3-(2-chlorophenyl)sulfanyl-6-[6-[3-(difluoromethyl)-4-fluoro-phenoxy]-2-pyridyl]-6-(3-thienyl)piperidine-2,4-dione;
3-(2-chlorophenyl)sulfanyl-6-[6-[3-(difluoromethyl)phenoxy]-2-pyridyl]-6-(3-thienyl)piperidine-2,4-dione;
3-(2-chlorophenyl)sulfanyl-6-[6-[3-(hydroxymethyl)anilino]-2-pyridyl]-6-(3-thienyl)piperidine-2,4-dione;
3-(2-chlorophenyl)sulfanyl-6-[6-[3-(hydroxymethyl)-N-methyl-anilino]-2-pyridyl]-6-(3-thienyl)piperidine-2,4-dione;
3-(2-chlorophenyl)sulfanyl-6-[6-[3-fluoro-5-(hydroxymethyl)phenoxy]-2-pyridyl]-6-(3-thienyl)piperidine-2,4-dione;
3-(2-chlorophenyl)sulfanyl-6-[6-[4-fluoro-3-(hydroxymethyl)anilino]-2-pyridyl]-6-(3-thienyl)piperidine-2,4-dione;
3-(2-chlorophenyl)sulfanyl-6-[6-[4-fluoro-3-(trifluoromethyl)phenoxy]-2-pyridyl]-6-(3-thienyl)piperidine-2,4-dione;
3-(2-chlorophenyl)sulfanyl-6-[6-[6-(hydroxymethyl)indolin-1-yl]-2-pyridyl]-6-(3-thienyl)piperidine-2,4-dione;
3-(2-chlorophenyl)sulfanyl-6-[6-[N-methyl-3-(trifluoromethyl)anilino]-2-pyridyl]-6-(3-thienyl)piperidine-2,4-dione;
3-(2-chlorophenyl)sulfanyl-6-phenyl-6-(3-thienyl)piperidine-2,4-dione;
3-(2-chlorophenyl)sulfanyl-6-phenyl-6-thiazol-4-yl-piperidine-2,4-dione;
3-(2-chlorophenyl)sulfanyl-6-thiazol-4-yl-6-(3-thienyl)piperidine-2,4-dione;
4-[3-[5-(2-chlorophenyl)sulfanyl-2-(4-morpholinophenyl)-4,6-dioxo-2-piperidyl]phenyl]-N,N-dimethyl-benzenesulfonamide;
4-[3-[5-(2-chlorophenyl)sulfanyl-4,6-dioxo-2-(3-thienyl)-2-piperidyl]phenyl]-N,N-dimethyl-benzenesulfonamide;
4-[6-[5-(2-chlorophenyl)sulfanyl-4,6-dioxo-2-(3-thienyl)-2-piperidyl]-2-pyridyl]-N,N-dimethyl-benzenesulfonamide;
6-(3-aminophenyl)-3-(2-chlorophenyl)sulfanyl-6-(3-thienyl)piperidine-2,4-dione;
6-(3-anilinophenyl)-3-(2-chlorophenyl)sulfanyl-6-(3-thienyl)piperidine-2,4-dione;
6-(3-bromo-4-morpholino-phenyl)-3-(2-chlorophenyl)sulfanyl-6-(3-thienyl)piperidine-2,4-dione;
6-(3-bromophenyl)-3-(2-chlorophenyl)sulfanyl-1-methyl-6-(3-thienyl)piperidine-2,4-dione;
6-(3-bromophenyl)-3-(2-chlorophenyl)sulfanyl-6-(3-thienyl)piperidine-2,4-dione;
6-(5-bromo-6-morpholino-3-pyridyl)-3-(2-chlorophenyl)sulfanyl-6-(3-thienyl)piperidine-2,4-dione;
6-(6-benzyl-2-pyridyl)-3-(2-chlorophenyl)sulfanyl-6-(3-thienyl)piperidine-2,4-dione;
6-(6-benzyloxy-2-pyridyl)-3-(2-chlorophenyl)sulfanyl-6-(3-thienyl)piperidine-2,4-dione;
6-(6-bromo-2-pyridyl)-3-(2-chloro-5-hydroxy-phenyl)sulfanyl-6-(3-thienyl)piperidine-2,4-dione;
6-(6-bromo-2-pyridyl)-3-(2-chlorophenyl)sulfanyl-6-(3-thienyl)piperidine-2,4-dione;
6-(6-bromo-5-morpholino-2-pyridyl)-3-(2-chlorophenyl)sulfanyl-6-(3-thienyl)piperidine-2,4-dione;
6-[3-chloro-5-(4-fluoroanilino)phenyl]-3-(2-chlorophenyl)sulfanyl-6-(3-thienyl)piperidine-2,4-dione;
6-[4-(1,3,3a,4,6,6a-hexahydrofuro[3,4-c]pyrrol-5-yl)phenyl]-3-(2-chlorophenyl)sulfanyl-6-(3-thienyl)piperidine-2,4-dione;
6-[4-(2-azaspiro[3.3]heptan-2-yl)phenyl]-3-(2-chlorophenyl)sulfanyl-6-(3-thienyl)piperidine-2,4-dione;
6-[4-(3-azabicyclo[2.1.1]hexan-3-yl)phenyl]-3-(2-chlorophenyl)sulfanyl-6-(3-thienyl)piperidine-2,4-dione;
6-[4-(4-acetylpiperazin-1-yl)phenyl]-3-(2-chlorophenyl)sulfanyl-6-(3-thienyl)piperidine-2,4-dione;
6-[5-(2-chlorophenyl)sulfanyl-4,6-dioxo-2-(3-thienyl)-2-piperidyl]-N-(cyclopropylmethyl)pyridine-2-carboxamide;
6-[6-(2-amino-5-methyl-imidazol-1-yl)-2-pyridyl]-3-(2-chlorophenyl)sulfanyl-6-(3-thienyl)piperidine-2,4-dione;
6-[6-(2-bromophenoxy)-2-pyridyl]-3-(2-chlorophenyl)sulfanyl-6-(3-thienyl)piperidine-2,4-dione;
6-[6-(2-chloro-3,4-difluoro-anilino)-2-pyridyl]-3-(2-chlorophenyl)sulfanyl-6-(3 thienyl)piperidine-2,4-dione;
6-[6-(2-chloro-4-fluoro-anilino)-2-pyridyl]-3-(2-chlorophenyl)sulfanyl-6-(3-thienyl)piperidine-2,4-dione;
6-[6-(2-chloro-4-fluoro-phenoxy)-2-pyridyl]-3-(2-chlorophenyl)sulfanyl-6-(3-thienyl)piperidine-2,4-dione;
6-[6-(2-tert-butoxyethoxy)-2-pyridyl]-3-(2-chlorophenyl)sulfanyl-6-(3-thienyl)piperidine-2,4-dione;
6-[6-(3-bromo-4-fluoro-phenoxy)-2-pyridyl]-3-(2-chlorophenyl)sulfanyl-6-(3-thienyl)piperidine-2,4-dione;
6-[6-(3-chloro-4-fluoro-anilino)-2-pyridyl]-3-(2-chlorophenyl)sulfanyl-6-(3-thienyl)piperidine-2,4-dione;
6-[6-(3-chloro-4-fluoro-phenoxy)-2-pyridyl]-3-(2-chlorophenyl)sulfanyl-6-(3-thienyl)piperidine-2,4-dione;
6-[6-(3-chlorophenoxy)-2-pyridyl]-3-(2-chlorophenyl)sulfanyl-6-(3-thienyl)piperidine-2,4-dione;
6-[6-(4-bromo-2-chloro-phenoxy)-2-pyridyl]-3-(2-chlorophenyl)sulfanyl-6-(3-thienyl)piperidine-2,4-dione;
6-[6-(4-bromo-2-fluoro-phenoxy)-2-pyridyl]-3-(2-chlorophenyl)sulfanyl-6-(3-thienyl)piperidine-2,4-dione;
6-[6-(4-chloro-N-methyl-anilino)-2-pyridyl]-3-(2-chlorophenyl)sulfanyl-6-(3-thienyl)piperidine-2,4-dione;
6-[6-(4-chlorophenoxy)-2-pyridyl]-3-(2-chlorophenyl)sulfanyl-6-(3-thienyl)piperidine-2,4-dione;
6-[6-(7-bromotetralin-1-yl)oxy-2-pyridyl]-3-(2-chlorophenyl)sulfanyl-6-(3-thienyl)piperidine-2,4-dione;
6-[6-[(2-chloro-6-fluoro-3-pyridyl)oxy]-2-pyridyl]-3-(2-chlorophenyl)sulfanyl-6-(3-thienyl)piperidine-2,4-dione;
6-[6-[(4-chloro-3-fluoro-phenyl)methyl]-2-pyridyl]-3-(2-chlorophenyl)sulfanyl-6-(3-thienyl)piperidine-2,4-dione;
6-[6-[[1-(3-chloro-4-fluoro-phenyl)-2-hydroxy-ethyl]amino]-2-pyridyl]-3-(2-chlorophenyl)sulfanyl-6-(3-thienyl)piperidine-2,4-dione;
6-[6-[1-(3-chloro-4-fluoro-phenyl)propylamino]-2-pyridyl]-3-(2-chlorophenyl)sulfanyl-6-(3-thienyl)piperidine-2,4-dione;
6-[6-[1-(4-chlorophenyl)ethoxy]-2-pyridyl]-3-(2-chlorophenyl)sulfanyl-6-(3-thienyl)piperidine-2,4-dione;
N-[6-[5-(2-chlorophenyl)sulfanyl-4,6-dioxo-2-(3-thienyl)-2-piperidyl]-2-pyridyl]azetidine-1-sulfonamide tert-butyl;
5-(2-chlorophenyl)sulfanyl-4-hydroxy-2-[4-(1-piperidyl)phenyl]-2-(3-thienyl)-1,3-dihydropyridin-6-one; and N-[6-[5-(2-chlorophenyl)sulfanyl-4,6-dioxo-2-(3-thienyl)-2-piperidyl]-2-pyridyl]carbamate.

12. A pharmaceutical composition comprising a compound of claim 1 and a therapeutically inert carrier.

13. The use of a compound according to claim 1 for the treatment or prophylaxis of cancer.

14. The use of a compound according to claim 1 as an LDHA inhibitor.

15. The use of a compound according to claim 1 for the preparation of a medicament for the treatment or prophylaxis of cancer.

16. A compound according to claim 1 for use in the inhibition of LDHA.

17. A compound according to claim 1 for use against hypoxic and/or highly glycolytic tumors.

18. A compound according to claim 1 for use in the inhibition of cell survival.

19. A compound according to claim 1 for use in the treatment or prevention of cancer.

20. A method for the treatment or prophylaxis of cancer which method comprises administering an effective amount of a compound as defined in claim 1.
